(19) United States
(12) Patent Application Publication
(10) Pub. No.: US 2007/0149508 A1
(43) Pub. Date: Jun. 28, 2007

(54) Six membered heteroaromatic inhibitors targeting resistant kinase mutations

(75) Inventors: Glenn Noronha, Oceanside, CA (US); Jianguo Cao, San Diego, CA (US); Binqi Zeng, San Diego, CA (US); Chi Ching Mak, San Diego, CA (US); Andrew McPherson, San Diego, CA (US); Joel Renick, San Diego, CA (US); Ved P. Pathak, San Diego, CA (US); Chun Chow, San Diego, CA (US); Moorthy Palanki, Encinitas, CA (US); Richard M. Soll, San Diego, CA (US); Daniel L. Lohse, San Diego, CA (US); John D. Hood, San Diego, CA (US); Elena Dneprovskaia, San Diego, CA (US)

Correspondence Address: DLA PIPER US LLP, 4365 EXECUTIVE DRIVE, SUITE 1100, SAN DIEGO, CA, 92121-2133, US

(73) Assignee: TargeGen, Inc., San Diego, CA (US)

(21) Appl. No.: 11/591,076

(22) Filed: Oct. 31, 2006

Related U.S. Application Data
(60) Provisional application No. 60/733,115, filed on Nov. 2, 2005.

(51) Int. Cl. A61K 31/551; A61K 31/53; A61K 31/506; C07D 403/02

(52) U.S. Cl. 514/218; 514/242; 514/252.14; 514/256; 540/575; 544/295; 544/333; 544/182

(57) ABSTRACT

A compound is provided, having the general structure (A): 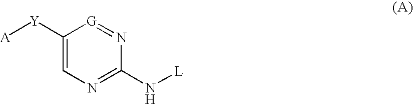 wherein A is an (un)substituted aryl or (un)substituted heteroaryl moiety, G is N, CH, or CR, R is an unsubstituted or substituted lower alkyl, Y is a hydrophobic linking moiety, and L is a substitutent as defined. The compound (A) can be used for treatment of various angiogenic and hematological-associated disorders, such as myeloproliferative disorder in patients who do not respond to kinase-inhibition therapy that comprises administering approved medications.

### CROSS REFERENCE TO RELATED APPLICATIONS

This application claims the benefit of priority under 35 U.S.C. § 119(e) of U.S. Ser. No. 60/733,115 filed Nov. 2, 2005, the entire content of which is incorporated herein by reference.

### BACKGROUND

1. Field of Invention

The present invention relates to the field of inhibitors of protein tyrosine kinases, their pharmaceutically acceptable compositions comprising the compounds of the invention and the methods of using the compositions in the treatment of various disorders. In particular, the present invention relates to several kinase inhibitors that can access residues deep within the hydrophobic pockets of kinases, or access portions of a conserved aspartic acid-phenylalanine-glycine (DFG) loop adjacent to the hydrophobic pockets of kinases, or circumvent the gatekeeper mutation.

2. Background of the Invention

Drug treatment induced resistance is an emerging theme of great importance in the design of inhibitors targeting various important human disease states. For example, Imatinib mesylate (Gleevec, ST1571) has become the standard of care for the treatment of patients with chronic myeloid leukemia (CML). Although responses in the chronic phase tend to be durable, relapse after an initial response is common in patients with more advanced disease. Point mutations within the kinase domain (KD) of BCR-ABL are the most common mechanism of acquired drug resistance, found in 50% to 90% of such patients

These kinds of resistances are seen in other inhibitors of BCR-ABL used to treat CML, and in Gleevec resistant CML, as seen in the cases of Nilotinib (Tasigna, AMN-107) and Dasatinib (Sprycel, BMS-354825). Neither of these 2^{nd} generation, or follow-on compounds targets the kinase with the gatekeeper mutation.

Similar cases of resistance are seen in using Gleevec against other disease states, where different kinases are targeted by Gleevec-platelet-derived growth factor (PDGFR) for example. And resistance to other kinase inhibitors that are approved therapies is an emerging theme, as seen in the cases drug induced resistances to gefinib (Iressa) and erlotinib (Tarceva) that are used to target epidermal growth factor receptor (EGFR). Both of these inhibitors are approved therapies. Fairly common to all of these inhibitors is the inability of the inhibitors to target the kinase domain in the cases of the targeted protein having the so-called gatekeeper mutation.

Protein kinases are families of enzymes that catalyze the phosphorylation of specific residues in proteins, broadly classified into tyrosine and serine/threonine kinases. Inappropriate kinase activity, arising from mutation, over-expression, or inappropriate regulation, dys-regulation or de-regulation, as well as over- or under-production of growth factors or cytokines has been implicated in many diseases, including but not limited too cancer, cardiovascular diseases, allergies, asthma and other respiratory diseases, autoimmune diseases, inflammatory diseases, bone diseases, metabolic disorders, and neurological and neurodegenerative disorders such as Alzheimer's disease. Inappropriate kinase activity triggers a variety of biological cellular responses relating to cell growth, cell differentiation, survival, apoptosis, mitogenesis, cell cycle control, and cell mobility implicated in the aforementioned diseases.

The protein kinase super family of enzymes has emerged as an important class of targets for therapeutic intervention with small molecules due to dysregulated kinase activity in many pathological conditions including cancer. For example, Gleevec is the first protein tyrosine kinase inhibitor to be approved for the treatment of human malignancy by virtue of its inhibition of several tyrosine kinases such as ABL, KIT, and PDGFR. Treatment with Gleevec as a single agent has demonstrated remarkable clinical efficacy in CML. The tyrosine kinase EGFR has been targeted with small molecule inhibitors such as Tarceva and Iressa for the treatment of patients with non-small cell lung carcinoma (NSCLC). SU11248 (Sutent) is approved for the treatment of certain tumors through its multi-modal action on the tyrosine kinases including the vascular endothelial growth factor receptor (VEGFR), KIT, and PDGFR. Inhibition of other kinases with small molecule inhibitors include the tyrosine kinase FLT3 that is expressed on blasts in most cases of acute myeloid leukemia (AML), the tyrosine kinases FGFR1, FGFR3, c-FMS, JAK, and SYK in a range of malignant hematological disorders, and ALK, c-met, and RET in a host of solid tumors.

Kinases other than tyrosine kinases are targets of small molecule inhibitors. For example, BAY 43-9006 (Sorafenib) exhibits inhibition of the serine threonine kinase RAF for the treatment of solid tumor malignancies, as well as the tyrosine kinase, VEGFR. The lipid kinase PI3K is a potential kinase target for therapeutic intervention in a host of human cancers including colon, brain, breast, prostate, glioblastoma, melanoma, and endometrial carcinoma.

Inhibiting kinases with ATP-competitive kinase inhibitors blocks enzymatic activity of the kinases. Often treatment therapies result in drug resistance over a period of time. Quite often, drug resistance is largely on account of mutations that occur to prevent the pressures exerted by drug binding. Thus, despite success with Gleevec to treat CML through inhibition of the oncogene BCR-ABL, clinical resistance to the drug has been observed. Of the multiple mechanisms of drug resistance, mutations of the BCR-ABL kinase have been particularly problematic with 50-90% of the resistance to Gleevec arsing from mutations in the kinase domain.

A variety of the 2^{nd} generation agents mentioned above, such as Nilotinib, and Dasatinib are able to inhibit a large number of clinically relevant mutations. However, neither of these agents inhibits the T315I mutation, also known as the gatekeeper mutation, although this mutation is the largest singly occurring mutation to Gleevec mono-therapy, the current standard of care for CML. Mutation of the gatekeeper residue enables the protein to bind ATP and continue to function, while Gleevec is selectively rejected since it makes use of a hydrophobic pocket close to the ATP binding site, which ATP does not utilize. As a matter of fact, almost all small molecule inhibitors that are ATP-competitive utilize this hydrophobic pocket to attain much higher potency over ATP. Gleevec is no exception. It is therefore, not surprising that the gatekeeper and its mutation across numerous kinases is well known since most small molecule inhibitors of kinases are ATP competitive. Mutation of the gatekeeper residue confers resistance of kinases such as p38, SRC, EGFR to different ATP-competitive inhibitors, including SB203580, PP1, and PD153035, respectively. While mutations seem to be selectively enhanced under pressures from inhibitor molecules, the common theme of resistance to inhibitor molecules that serve as drugs is clearly emerging.

Preclinical and clinical data obtained to date suggest that, for human CML that is driven by BCR-ABL, the apparent ability of Dasatinib to bind to both the active and the inactive conformations of BCR-ABL affords this agent greater therapeutic potential compared with an agent, such as Gleevec, which binds only to an inactive form of the enzyme. Phase I and II clinical experience has shown promising results for Dasatinib in patients with Imatinib-resistant and Imatinib-intolerant disease. Current studies in human patients reveal that the potency and favorable profile of Dasatinib against wild-type ABL and several imatinib resistant ABL mutants is at least partially due to its ability to recognize multiple states of BCR-ABL. Yet, Dasatinib is till completely ineffective against the gatekeeper mutation, the single largest mutation arising from all existing therapies in CML.

The concept of using combination therapies to treat resistance to existing therapies makes use of the idea of different inhibitors exploring differing spaces and differing activation states of the kinases. Thus, low doses of 2^{nd} generation agents, Dasatinib or Nilotinib separately, or Dasatinib combined with low doses of Nilotinib may effectively suppress the emergence of almost all other mutations other than T315I. Because the nonhematologic side effects of Nilotinib and Dasatinib are not identical, patients with intolerance to either agent could be managed with combinations at low doses, avoiding toxicity while maintaining full antileukemic activity. Clearly, with T315I emerging as the mutation that is not targeted by any of these agents, an inhibitor of T315I is needed for patients that show resistance to all of these existing therapies.

A common structural theme, amongst kinases is the existence of particular pockets that are accessed by kinase inhibitors in both active and inactive states of the enzyme. Unlike ATP, which binds to the active site of all kinases, many small molecule kinase inhibitors derive their unusual potencies and specificities to particular pockets that are available to the inhibitor upon binding in addition to binding at ATP-binding residues. For example, the dual SRC and ABL inhibitor Dasatinib binds to this deep hydrophobic pocket defined by the protein in both SRC and ABL and does not form any key hydrogen bonding interactions within this deep specificity pocket. The gate-keeper residue, as the name describes, sits just at the entrance to this pocket and thus resistance to these inhibitors in large part, is conferred simply by mutations, especially at the gatekeeper residue. Dasatinib is completely un-effective against mutation of the gatekeeper T315I mutation.

Deep within the hydrophobic pocket is an acceptor residue, a glutamatic acid, forming a key salt bridge with a lysine (K295 in the case of SRC and K271 in the case of ABL). Other residues in close proximity are the Aspartic acid from the DFG portion of the activation loop and other conserved residues that are part of the activation mechanisms of these kinases. Despite their promixity and well-conserved nature across all kinases, kinase inhibitor design has failed in taking advantage of any of these key residues in any specific and targeted manner.

Clearly, with gatekeeper mutation resistance emerging in CML as the major mutation with combined and individual second generation therapies, an inhibitor of T315I remains an unmet need in CML. Approved inhibitors targeting CML and other disease states do not describe specific designs to make use of residues deep within and adjacent to the hydrophobic pockets in the kinase domains. Designs targeting the gatekeeper resistant proteins are not described with approved inhibitor series. Designs targeting the gatekeeper resistant proteins by targeting residues deep within and adjacent to the hydrophobic pockets in the kinase domains are not described for approved inhibitors. The concept of inhibitor design and examples targeting conserved yet uniquely positioned residues deep within and proximal to the hydrophobic pocket as a part of inhibitor design to circumvent the gatekeeper mutation is provided here.

This concept is the basis for the inhibitors targeting the gatekeeper mutation in CML, where resistance is seen to all the current therapies including Gleevec, Sprycel and Tasigna, or any other inhibitor that does not target the gatekeeper mutation resistant ABL or BCR-ABL protein effectively. The concept can be applied in designing inhibitors that bind other kinases with gatekeeper mutations, where mutations in the gatekeeper residue arise on treatment with Gleevec, Sprycel and Tasigna, when these inhibitors are used to target these kinases, and such resistance is manifested rendering these inhibitors less effective or ineffective.

This concept can be applied in other kinds of drug related resistance as in the case of gatekeeper mutation resistance kinases from Tarceva, Iressa, and all other approved kinase inhibitors that are approved as therapies for other treatment conditions.

### SUMMARY

According to one embodiment, a compound is provided, the compound having an aryl or heteroaryl moiety and a hydrophobic linking moiety connecting the aryl or heteroaryl moiety to a pyrimidine derived moiety or a triazine-derived moiety, where the aryl or heteroaryl moiety carries a first substitutent comprising an acidic proton, and the pyrimidine or triazine-derived moiety carries a second substitutent comprising a primary or secondary amino group. The compound can be used for treatment of various diseases, disorders, and pathologies, including treatment of hematological disorders, such as myeloproliferative disorders, including disorders such as chronic myelogenous leukemia (CML)

According to another embodiment, a method is provided for treatment of various diseases, disorders, and pathologies, including treatment of angiogenic or hematological associated disorders, such as myeloproliferative disorder, the methods to include determining, in a group of patients, which patients do not respond to treatments with Gleevec, or Nilotinib, or Dasatinib types of compounds and administering to such non-responding patients a compound of the present invention.

### DETAILED DESCRIPTION

A. Terms and Definitions.

The following terminology and definitions apply as used in the present application, generally in conformity with the terminology recommended by the International Union of Pure and Applied Chemistry (IUPAC):

The term “heteroatom” refers to any atom other than carbon, for example, N, O, or S.

The term “aromatic” refers to a cyclically conjugated molecular entity with a stability, due to delocalization, significantly greater than that of a hypothetical localized structure, such as the Kekule structure.

The term “heterocyclic,” when used to describe an aromatic ring, refers to the aromatic rings containing at least one heteroatom, as defined above.

The term “heterocyclic,” when not used to describe an aromatic ring, refers to cyclic (i.e., ring-containing) groups other than aromatic groups, the cyclic group being formed by between 3 and about 14 carbon atoms and at least one heteroatom described above.

The term “substituted heterocyclic” refers, for both aromatic and non-aromatic structures, to heterocyclic groups further bearing one or more substituents described below.

The term “alkyl” refers to a monovalent straight or branched chain hydrocarbon group having from one to about 12 carbon atoms, for example, methyl, ethyl, n-propyl, iso-propyl, n-butyl, iso-butyl, tert-butyl, n-pentyl (also known as n-amyl), n-hexyl, and the like. The term “lower alkyl” refers to alkyl groups having from 1 to about 6 carbon atoms.

The term “substituted alkyl” refers to alkyl groups further bearing one or more substituents such as hydroxy, alkoxy, mercapto, cycloalkyl, substituted cycloalkyl, heterocyclic, substituted heterocyclic, aryl, substituted aryl, heteroaryl, substituted heteroaryl, aryloxy, substituted aryloxy, halogen, cyano, nitro, amino, amido, aldehyde, acyl, oxyacyl, carboxyl, sulfonyl, sulfonamide, sulfuryl, and the like.

The term “alkenyl” refers to straight-chained or branched hydrocarbyl groups having at least one carbon-carbon double bond, and having between about 2 and about 12 carbon atoms, and the term “substituted alkenyl” refers to alkenyl groups further bearing one or more substituents described above.

The term “alkynyl” refers to straight-chained or branched hydrocarbyl groups having at least one carbon-carbon triple bond, and having between about 2 and about 12 carbon atoms, and the term “substituted alkynyl” refers to alkynyl groups further bearing one or more substituents described above.

The term “aryl” refers to aromatic groups having between about 5 and about 14 carbon atoms and the term “substituted aryl” refers to aryl groups further bearing one or more substituents described above.

The term “heteroaryl” refers to aromatic rings, where the ring structure is formed by between 3 and about 14 carbon atoms and by at least one heteroatom described above, and the term “substituted heteroaryl” refers to heteroaryl groups further bearing one or more substituents described above.

The term “alkoxy” refers to the moiety —O-alkyl, wherein alkyl is as defined above, and the term “substituted alkoxy” refers to alkoxy groups further bearing one or more substituents described above.

The term “cycloalkyl” refers to alkyl groups having between 3 and about 8 carbon atoms arranged as a ring, and the term “substituted cycloalkyl” refers to cycloalkyl groups further bearing one or more substituents described above.

The term “alkylaryl” refers to alkyl-substituted aryl groups and the term “substituted alkylaryl” refers to alkylaryl groups further bearing one or more substituents described above.

The term “arylalkyl” refers to aryl-substituted alkyl groups and the term “substituted arylalkyl” refers to arylalkyl groups further bearing one or more substituents described above.

The term “arylalkenyl” refers to aryl-substituted alkenyl groups and the term “substituted arylalkenyl” refers to arylalkenyl groups further bearing one or more substituents described above.

The term “arylalkynyl” refers to aryl-substituted alkynyl groups and the term “substituted arylalkynyl” refers to arylalkynyl groups further bearing one or more substituents described above.

The term “arylene” refers to divalent aromatic groups having between 5 and about 14 carbon atoms and the term “substituted arylene” refers to arylene groups further bearing one or more substituents described above.

The term “kinase” refers to any enzyme that catalyzes the addition of phosphate groups to a protein residue; for example, serine and threonine kinases catalyze the addition of phosphate groups to serine and threonine residues.

The term “therapeutically effective amount” refers to the amount of the compound or pharmaceutical composition that will elicit the biological or medical response of a tissue, system, animal or human that is being sought by the researcher, veterinarian, medical doctor or other clinician, e.g., restoration or maintenance of vasculostasis or prevention of the compromise or loss or vasculostasis; reduction of tumor burden; reduction of morbidity and/or mortality.

The term “pharmaceutically acceptable” refers to the fact that the carrier, diluent or excipient must be compatible with the other ingredients of the formulation and not deleterious to the recipient thereof.

The terms “administration of a compound” or “administering a compound” refer to the act of providing a compound of the invention or pharmaceutical composition to the subject in need of treatment.

The term “antibody” refers to intact molecules of polyclonal or monoclonal antibodies, as well as fragments thereof, such as Fab and F(ab′)₂, Fv and SCA fragments which are capable of binding an epitopic determinant.

B. Embodiments of the Invention.

According to an embodiment of the invention, compounds are provided for treatment of various diseases, disorders, and pathologies, including treatment of angiogenic-associated disorders, such as myeloproliferative disorder. The compounds include an aryl or a heteroaryl moiety and a hydrophobic linking moiety connecting the aryl or heteroaryl moiety to a pyrimidine derived moiety or a triazine-derived moiety.

The aryl or heteroaryl moiety carries a first substitutent comprising an acidic proton, such as hydroxyl, carboxyl, amino, or amido group, which can be attached to any position of the aryl or heteroaryl moiety as chemically reasonable. The pyrimidine- or benzotirazine-derived moiety carries a second substitutent comprising a primary or secondary amino group.

Schematically, therefore, the compounds of the present invention can be represented as the general structure (A): 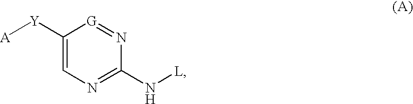
wherein G is N, CH, or CR, R is an unsubstituted or substituted lower alkyl, A is an aryl or heteroaryl group, as discussed below, Y is a hydrophobic two carbon linking moiety, and L is:

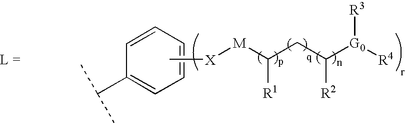

Some examples of specific moieties that can represent an aryl or heteroaryl moiety A shown in the structure (A), can include, but are not limited to, one of the following moieties:

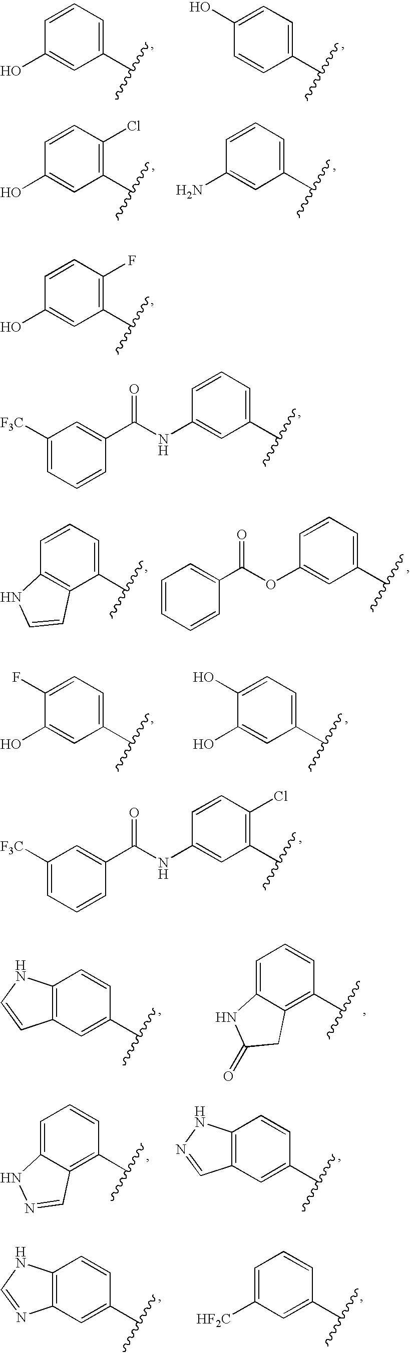

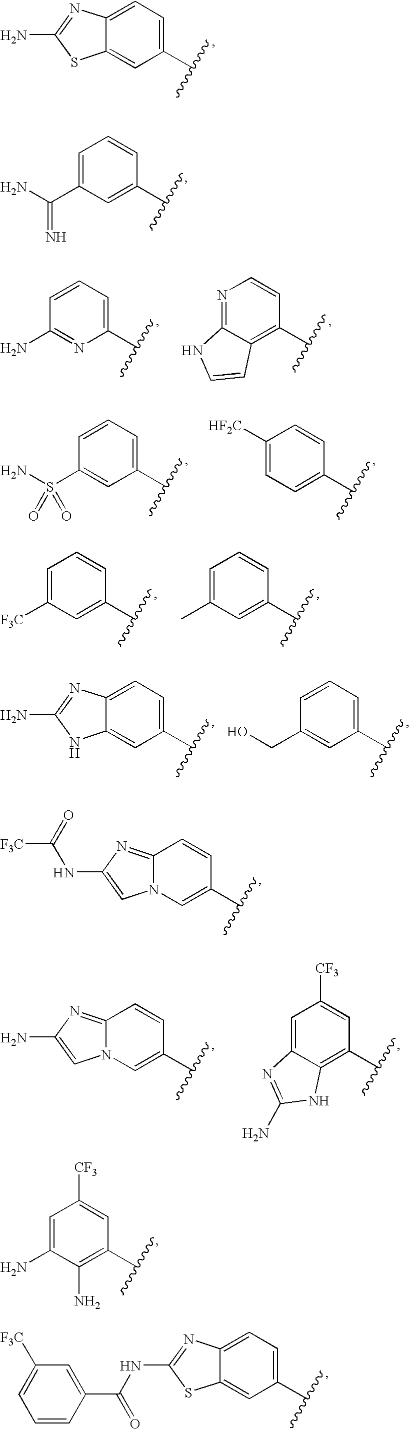

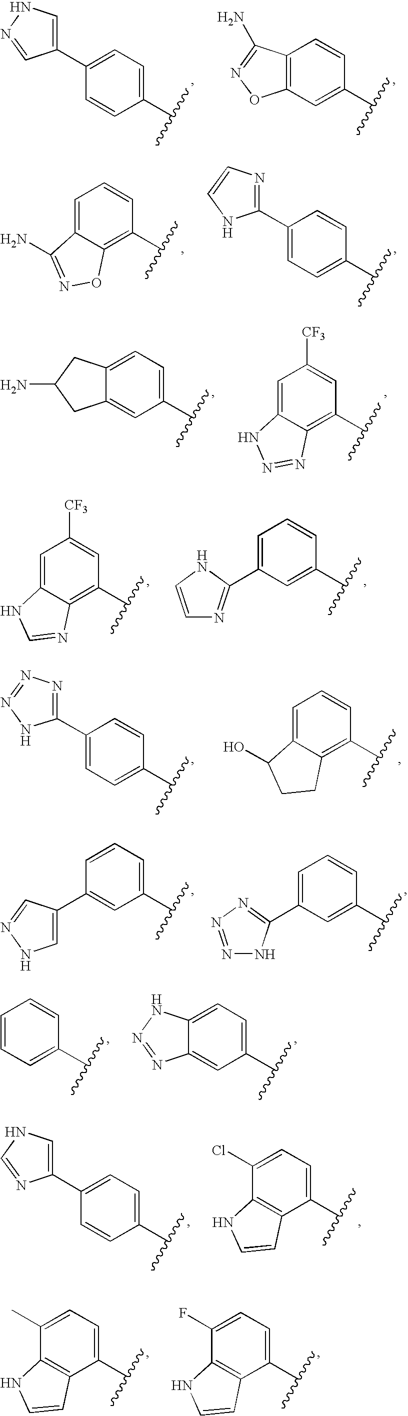

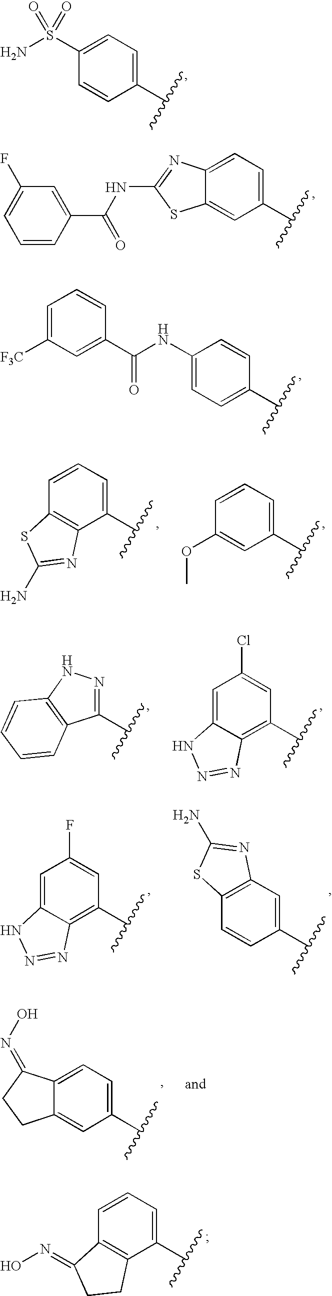

The above-mentioned linking moiety Y shown in the structure (A) can be attached to any position of the aryl or heteroaryl moiety A, and to any position of the pyrimidine- or triazine-derived moiety, as chemically reasonable. The linking moiety Y that can be used includes an alkyl or an alkylene group, such as a group shown below:

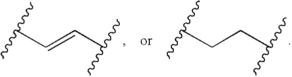

The above-mentioned moiety Y shown in the structure (A) can be attached to any position of the aryl or heteroaryl moiety A, and to any position of the pyrimidine- or triazine-derived moiety, as chemically reasonable, and moiety L is: 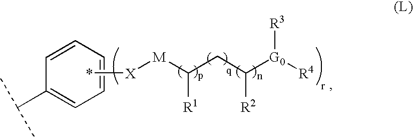
wherein X can be any of a bond, O, C═O, SO₂, or CH₂ and M can be a bond or NR⁹; or X and Y taken together can be a bond. Further, in the structure (A) each of R¹ and R² can be any of H, CF₃, F, Cl, Br, I, OH, OCH₃, CN, OCF₃, NH₂, C₁-C₆ substituted or unsubstituted alkyl, substituted or unsubstituted cycloalkyl, substituted or unsubstituted heterocycle, substituted or unsubstituted aryl, or substituted or unsubstituted heteroaryl; or R¹ and R² taken together can be a bond; or R¹ and R² taken together can form a moiety such as one of (CH₂)ₘ, (CH₂)ᵣ—S—(CH₂)ₘ, (CH₂)ᵣ—SO—(CH₂)ₘ, (CH₂)ᵣ—SO₂—(CH₂)ₘ, (CH₂)ᵣ—NR⁹—(CH₂)ₘ, or (CH₂)ᵣ—O—(CH₂)ₘ, wherein each of p, q, r, n, m is independently an integer having the value between 0 and 6.

Further, in the structure (A) R⁹ can be one of H, C₁-C₆ substituted or unsubstituted alkyl, C₁-C₆ substituted or unsubstituted alkenyl, C₁-C₆ substituted or unsubstituted alkynyl, C₁-C₆ substituted or unsubstituted hydroxyalkyl or aminoalkyl, C₁-C₆ substituted or unsubstituted branched alkyl, C₁-C₆ substituted or unsubstituted cycloalkyl, substituted or unsubstituted aryl connected through carbon or a heteroatom, substituted or unsubstituted heteroaryl connected through carbon or a heteroatom, C₁-C₆ alkoxy, a halogen, CF₃, —OCF₃, CHR³R⁴, SR³, SOR³, SO₂R³, SO₂NR³R⁴, SO₃R³, POR³, PO₂R³, PO₂NR³R⁴, PO₂CR³R⁴, PO₃R³, NR³R⁴, NO₂, CN, OH, CONR³R⁴, COR³, COOR³ NR³COR⁴, NR³CONR³R⁴, OCONR³R⁴, CSNR³R⁴, CSR³, NR³CSNR³R⁴, SCONR³R⁴, SCSNR³R⁴, or SCSNR³R⁴; G₀ can be one of N, O, H, of CH, with the proviso that if G₀ is N, then each of R³ and R⁴ can be one of H, CF₃, F, Cl, Br, I, OH, OCH₃, CN, OCF₃, NH₂, C₁-C₆ alkyl, C₁-C₆ substituted or unsubstituted hydroxyalkyl or aminoalkyl, C₁-C₆ substituted or unsubstituted branched alkyl, substituted or unsubstituted aryl, or substituted or unsubstituted heteroaryl, or R³ and R⁴ taken together can form a moiety such as one of (CH₂)ₘ, (CH₂)ᵣ—S—(CH₂)ₘ, (CH₂)ᵣ—SO—(CH₂)ₘ, (CH₂)ᵣ—SO₂—(CH₂)ₘ, (CH₂)ᵣ—NR⁹—(CH₂)ₘ, or (CH₂)ᵣ—O(CH₂)ₘ.

There are some additional provisos further directed to G₀ in the structure (A). More specifically, if G₀ is N, then R¹ and R⁹ taken together can form a moiety such as one of (CH₂)ₘ, (CH₂)ᵣ—S—(CH₂)ₘ, (CH₂)ᵣ—SO—(CH₂)ₘ, CH₂)ᵣ—SO₂—(CH₂)ₘ, (CH₂)ᵣ—NR⁹—(CH₂)ₘ, or (CH₂)C—O—(CH₂)ₘ; or R¹ and R⁴ taken together can form a moiety such as one of (CH₂)ₘ, (CH₂)ᵣ—S—(CH₂)ₘ, (CH₂)ᵣ—SO—(CH₂)ₘ, (CH₂)ᵣ—SO₂—(CH₂)ₘ, (CH₂)ᵣ—NR⁹—(CH₂)ₘ, or (CH₂)ᵣ—O—(CH₂)ₘ; or R⁹ and R⁴ taken together can form a moiety such as one of (CH₂)ₘ, (CH₂)ᵣ—S—(CH₂)ₘ, (CH₂)ᵣ—SO—(CH₂)ₘ, (CH₂)ᵣ—SO₂—(CH₂)ₘ, (CH₂)ᵣ—NR⁹—(CH₂)ₘ, or (CH₂)ᵣ—O—(CH₂)ₘ; or R³ and R⁴ taken together can form a moiety such as one of (CH₂)ₘ, (CH₂)ᵣ—S—(CH₂)ₘ, (CH₂)ᵣ—SO—(CH₂)ₘ, (CH₂)ᵣ—SO₂—(CH₂)ₘ, (CH₂)ᵣ—NR⁹—(CH₂)ₘ, or (CH₂)ᵣ—O (CH₂)ₘ.

If in the structure (A) G₀ is O, then R³ can be one of H, CF₃, F, Cl, Br, I, OH, OCH₃, CN, OCF₃, NH₂, C₁-C₆ alkyl and C₁-C₆ substituted or unsubstituted hydroxyalkyl or aminoalkyl, substituted or unsubstituted branched alkyl, substituted or unsubstituted cycloalkyl, substituted heterocyclic connected through carbon or nitrogen, substituted or unsubstituted aryl, or substituted or unsubstituted heteroaryl connected through carbon or nitrogen, with no group R⁴; R¹ and R⁹ taken together can form a moiety such as one of (CH₂)ₘ, (CH₂)ᵣ—S—(CH₂)ₘ, (CH₂)ᵣ—SO—(CH₂)ₘ, (CH₂)ᵣ—SO₂—(CH₂)ₘ, (CH₂)ᵣ—NR⁹—(CH₂)ₘ, or (CH₂)ᵣ—O—(CH₂)ₘ; or R¹ and R³ taken together can form a moiety such as one of (CH₂)ₘ, (CH₂)ᵣ—S—(CH₂)ₘ, (CH₂)ᵣ—SO(CH₂)ₘ, (CH₂)ᵣ—SO₂—(CH₂)ₘ, (CH₂)ᵣ—NR⁹—(CH₂)ₘ, or (CH₂)ᵣ—O—(CH₂)ₘ; or R⁹ and R³ taken together can form a moiety such as one of (CH₂)ₘ, (CH₂)ᵣ—S—(CH₂)ₘ, (CH₂)ᵣ—SO—(CH₂)ₘ, (CH₂)ᵣ—SO₂—(CH₂)ₘ, (CH₂)ᵣ—NR⁹—(CH₂)ₘ, or (CH₂)ᵣ—O—(CH₂)ₘ.

If in the structure (A), G₀=CH, then each of R³ and R⁴ can be one of H, CF₃, F, Cl, Br, I, OH, OCH₃, CN, OCF₃, NH₂, C₁-C₆ alkyl, C₁-C₆ substituted or unsubstituted hydroxyalkyl or aminoalkyl, C₁-C₆ substituted or unsubstituted branched alkyl, substituted or unsubstituted aryl, C₁-C₆ substituted or unsubstituted heterocycle connected through carbon or nitrogen, or substituted or unsubstituted heteroaryl connected through carbon or nitrogen, or R³ and R⁴ taken together can form a moiety such as one of (CHR⁹)ᵣ—(CHR⁹)ₘ—(CHR⁹)ₚ, (CHR⁹)ᵣ—S—(CHR⁹)ₘ, (CHR⁹)ᵣ—SO—(CHR⁹)ₘ, (CHR⁹)ᵣ—SO₂—(CHR⁹)ₘ, (CHR⁹)ᵣ—NR⁹—(CHR⁹)ₘ, or (CHR⁹)ᵣ—O—(CHR⁹)ₘ.

The aryl or heteroaryl moiety A shown in the structure (A), that can be used in the compounds of the invention can be any of and can included such as exemplary moieties as the moieities derived from benzene (e.g., benzene itself, phenol, toluene, phenylmethanol, chlorophenol, fluorophenol, halogenated alkyl benzene, aniline, benzamide, benziamines, benzoate, pyrocatechol, benzimide, or benzenesulfonamide), or from indole, indoline, indene, indazole, imidazole, benzothiazole, pyrazole, pyridine, pyrrolopyridine, benzimidazole, imidazopyridine, benzoisoxazole, phenylimidazole, benzotriazole, tetrazole, or anisole.

The above-mentioned substitutent L, as shown in the structure (A), that can be used in the compounds of the invention includes, but is not limited, to one of the following moieties:

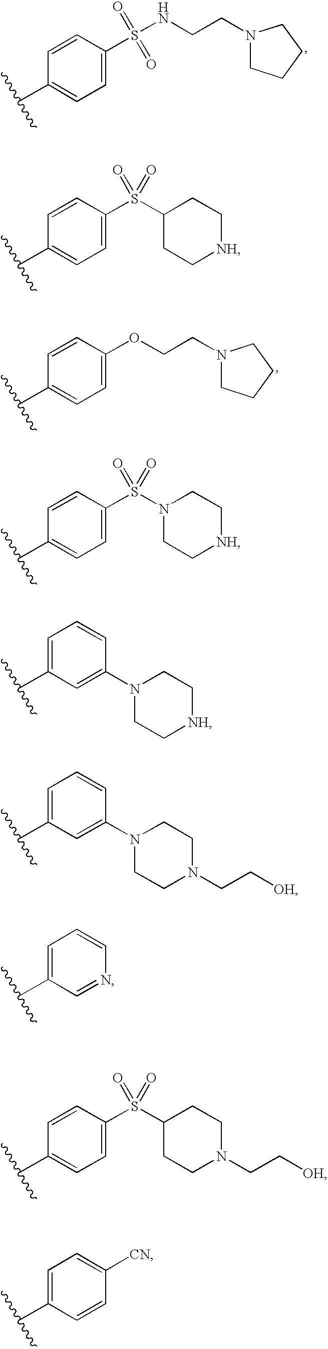

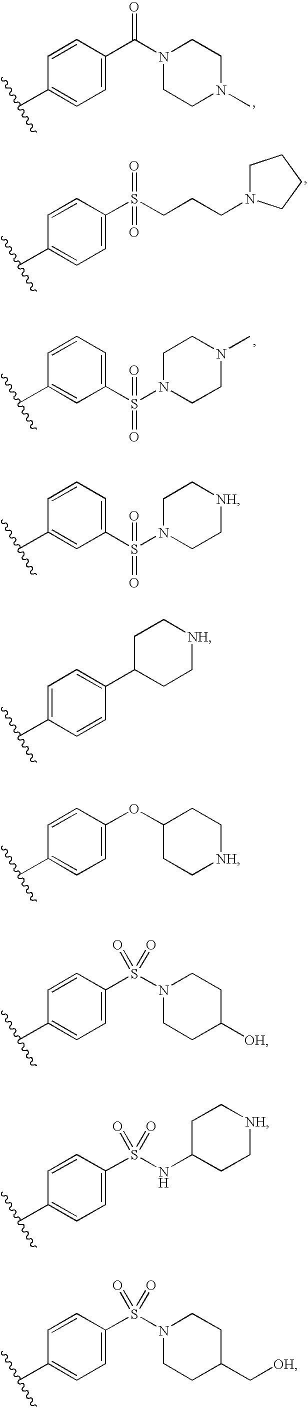

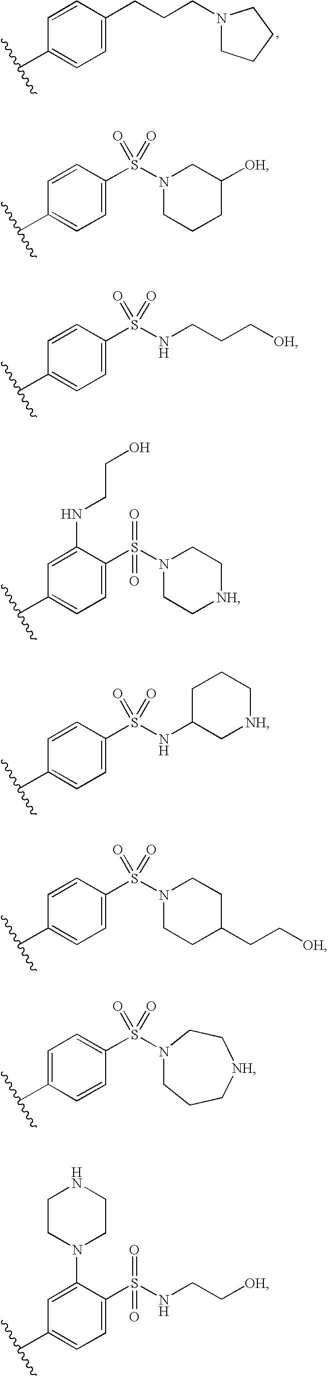

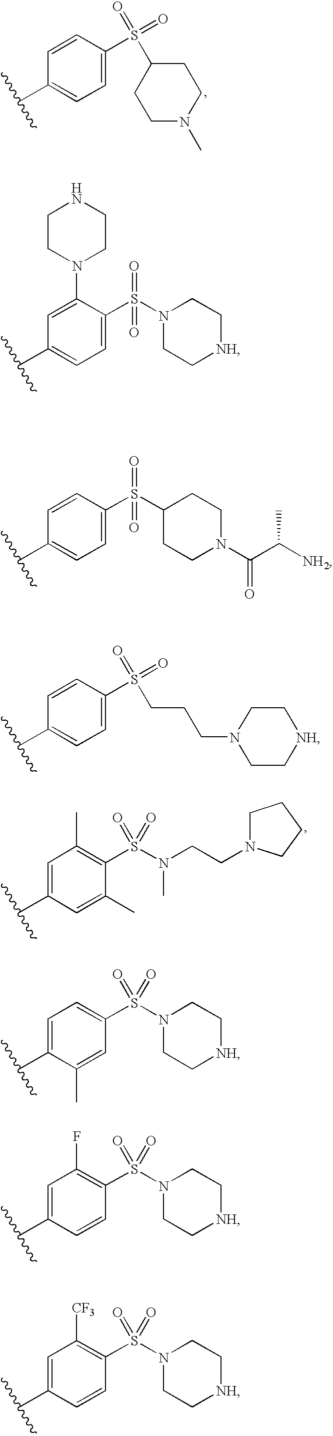

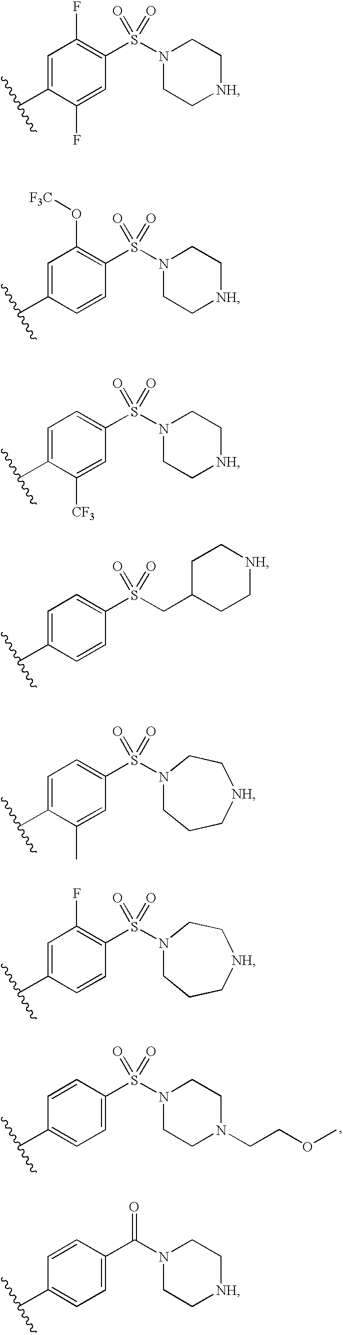

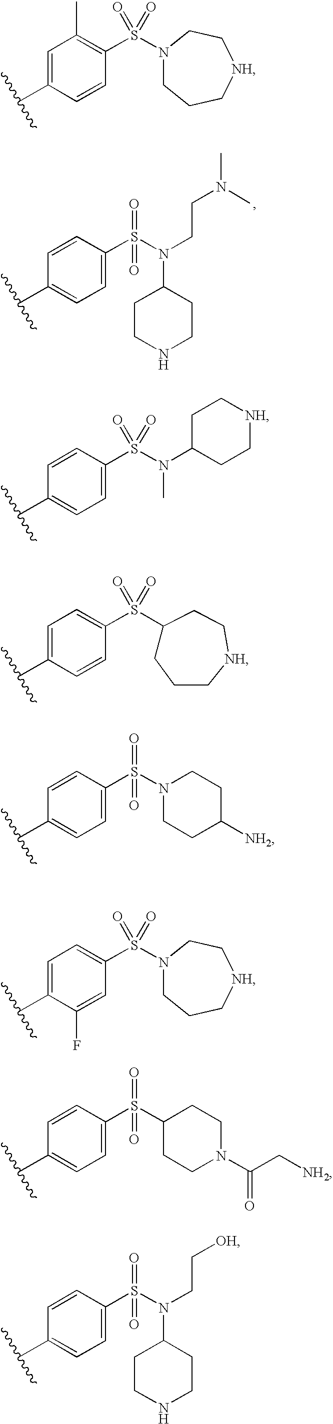

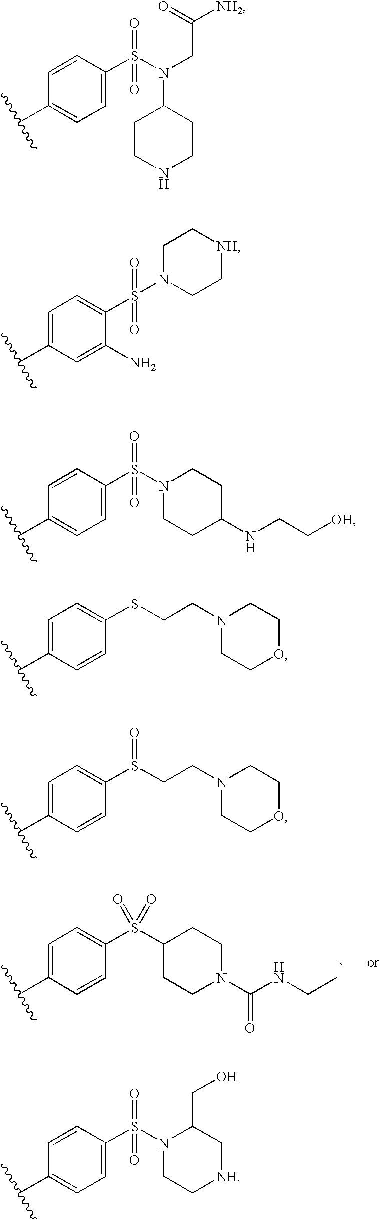

Some exemplary compounds described by structure (A) that can be used include, but are not limited to, the following compounds (I) through (CLXV) shown below:

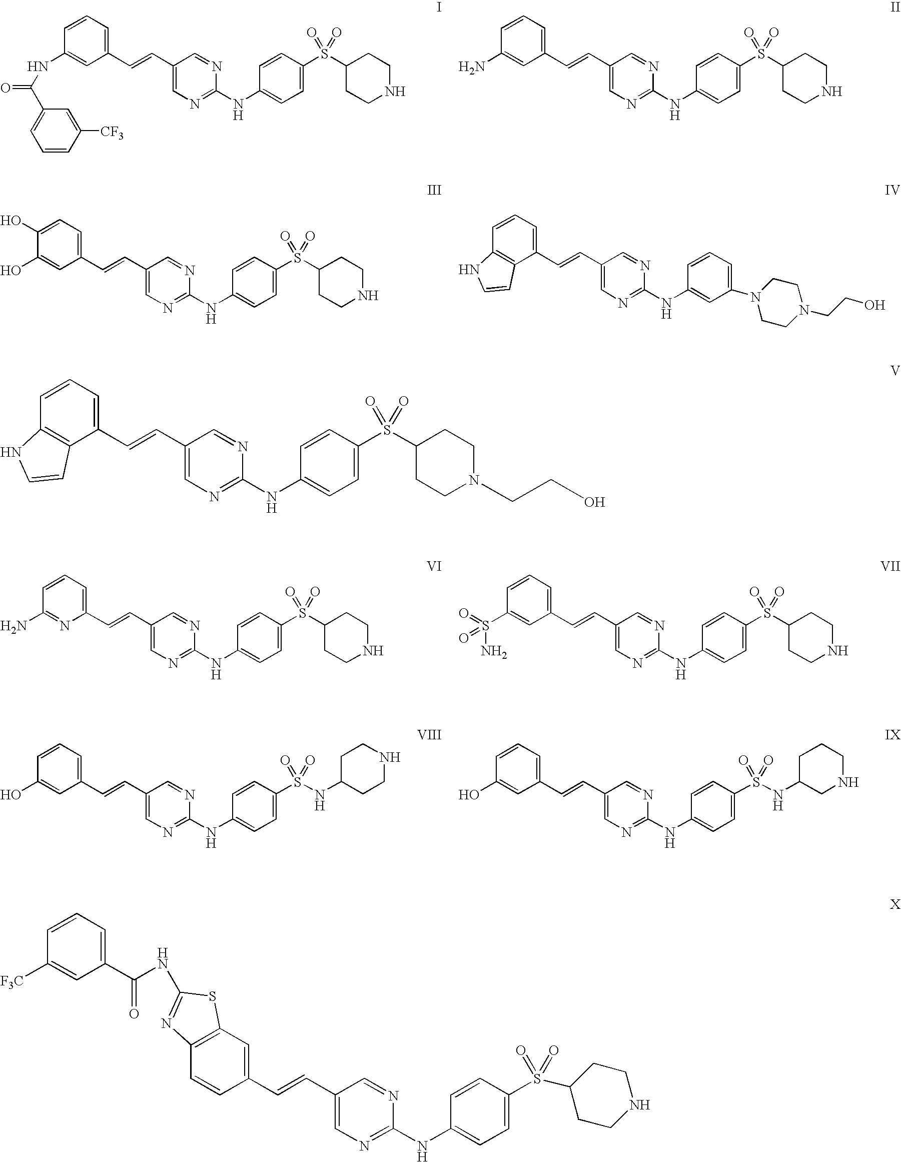

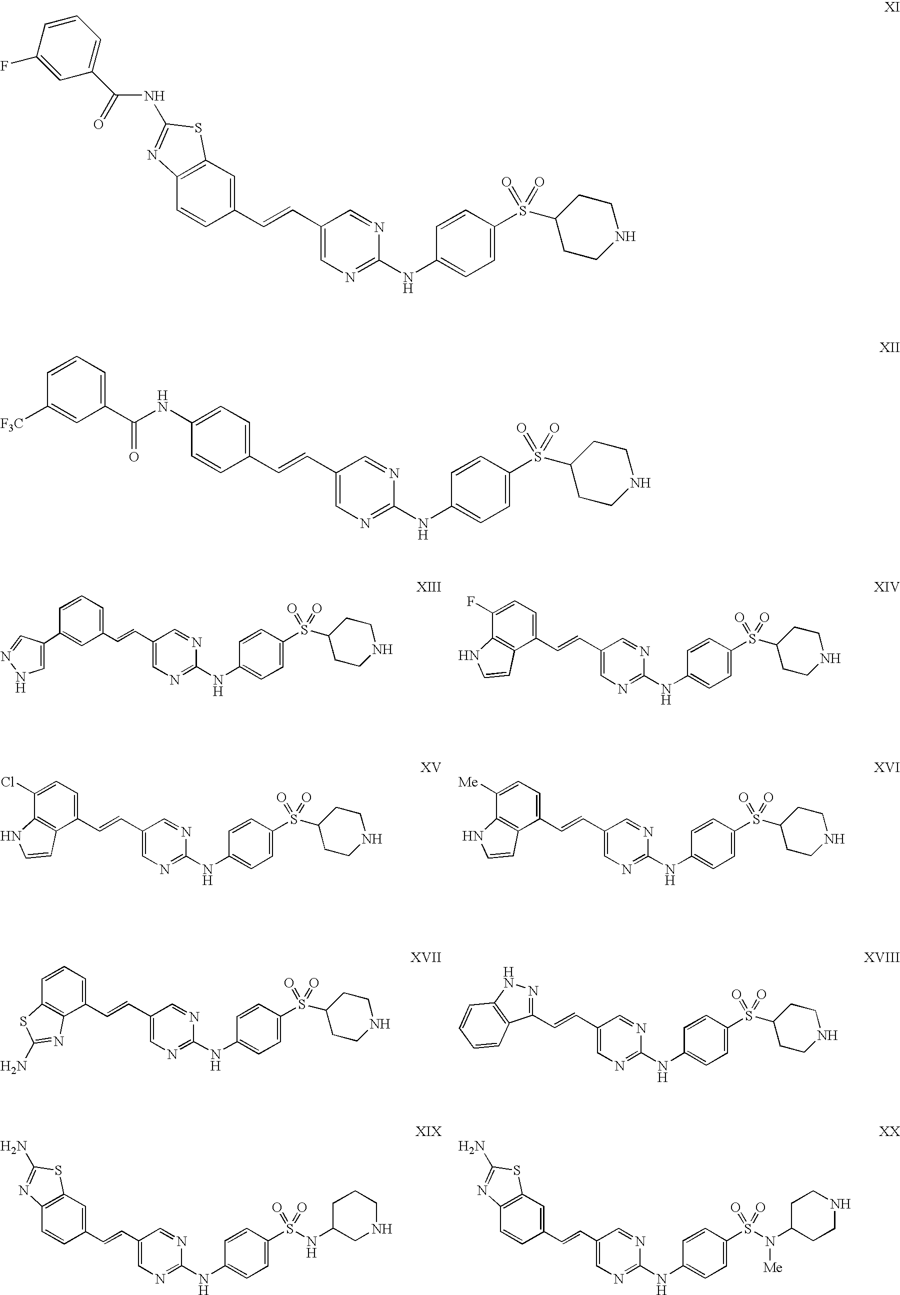

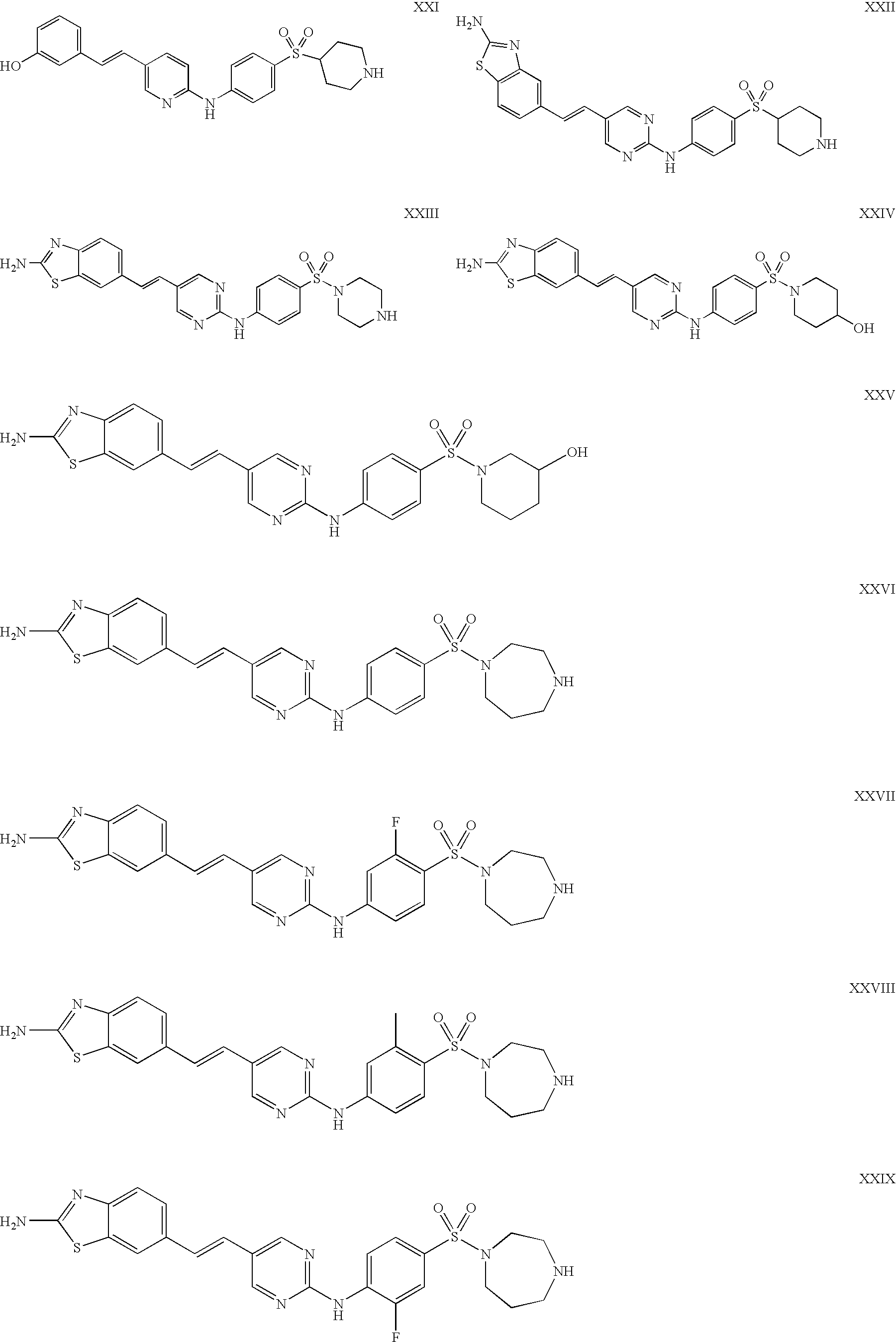

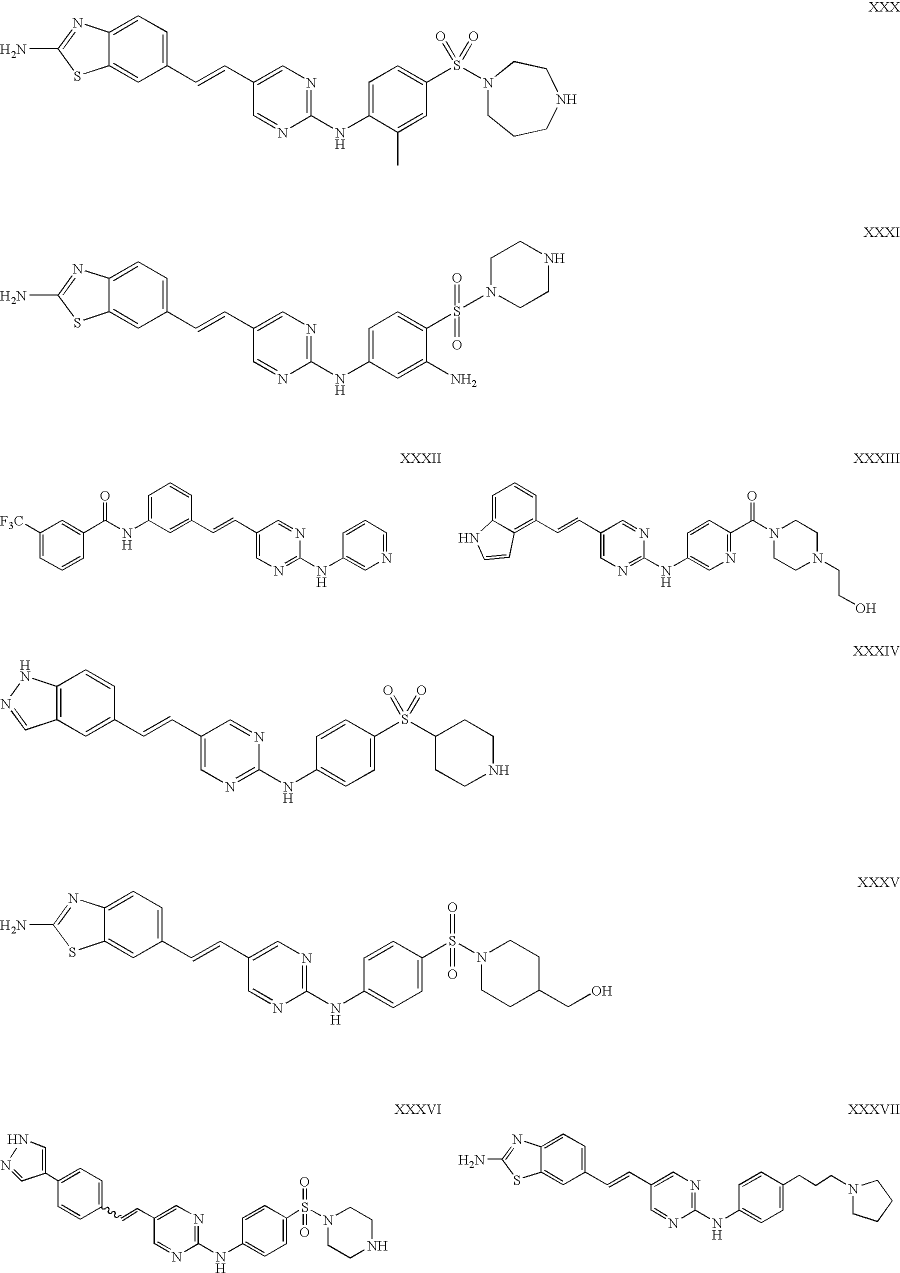

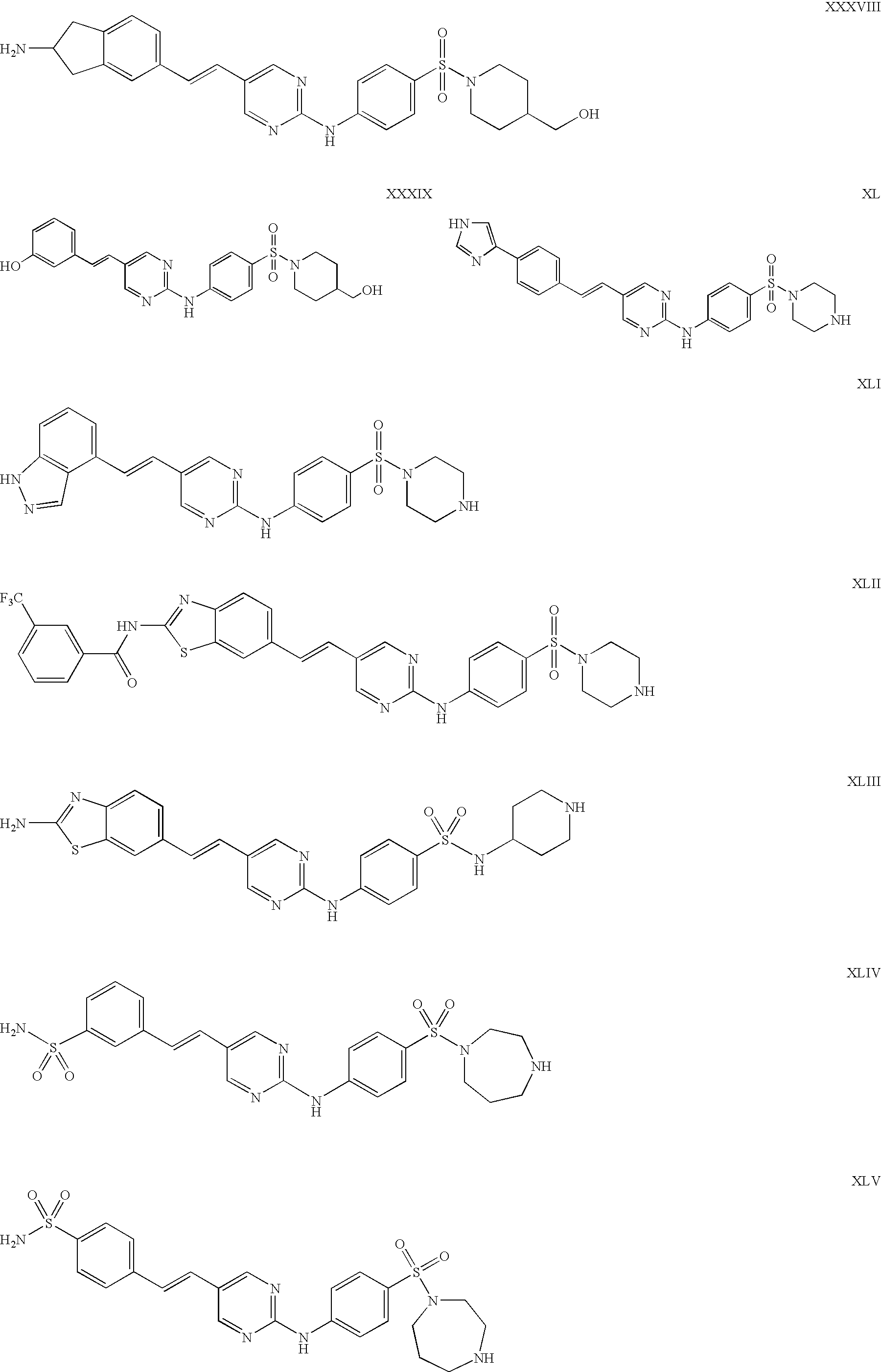

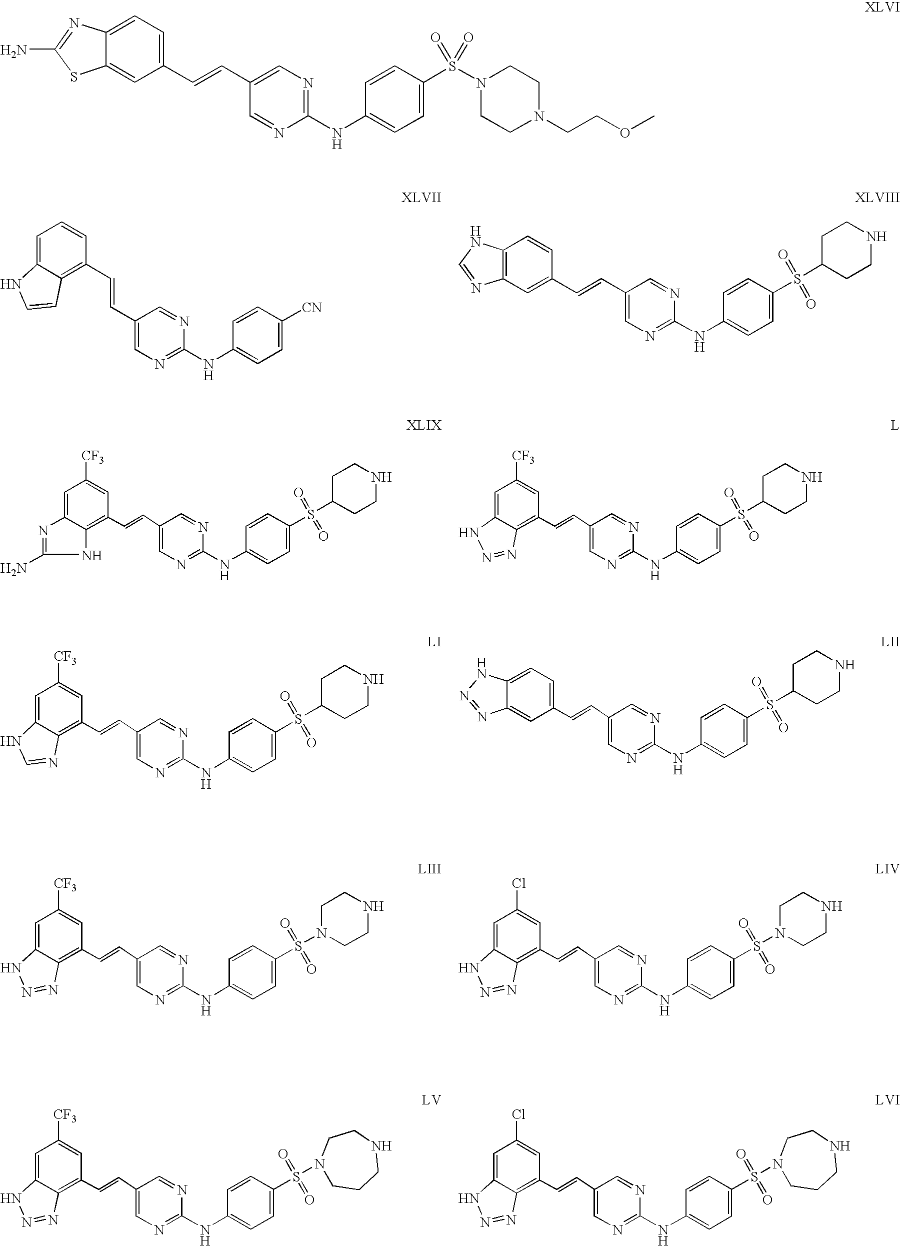

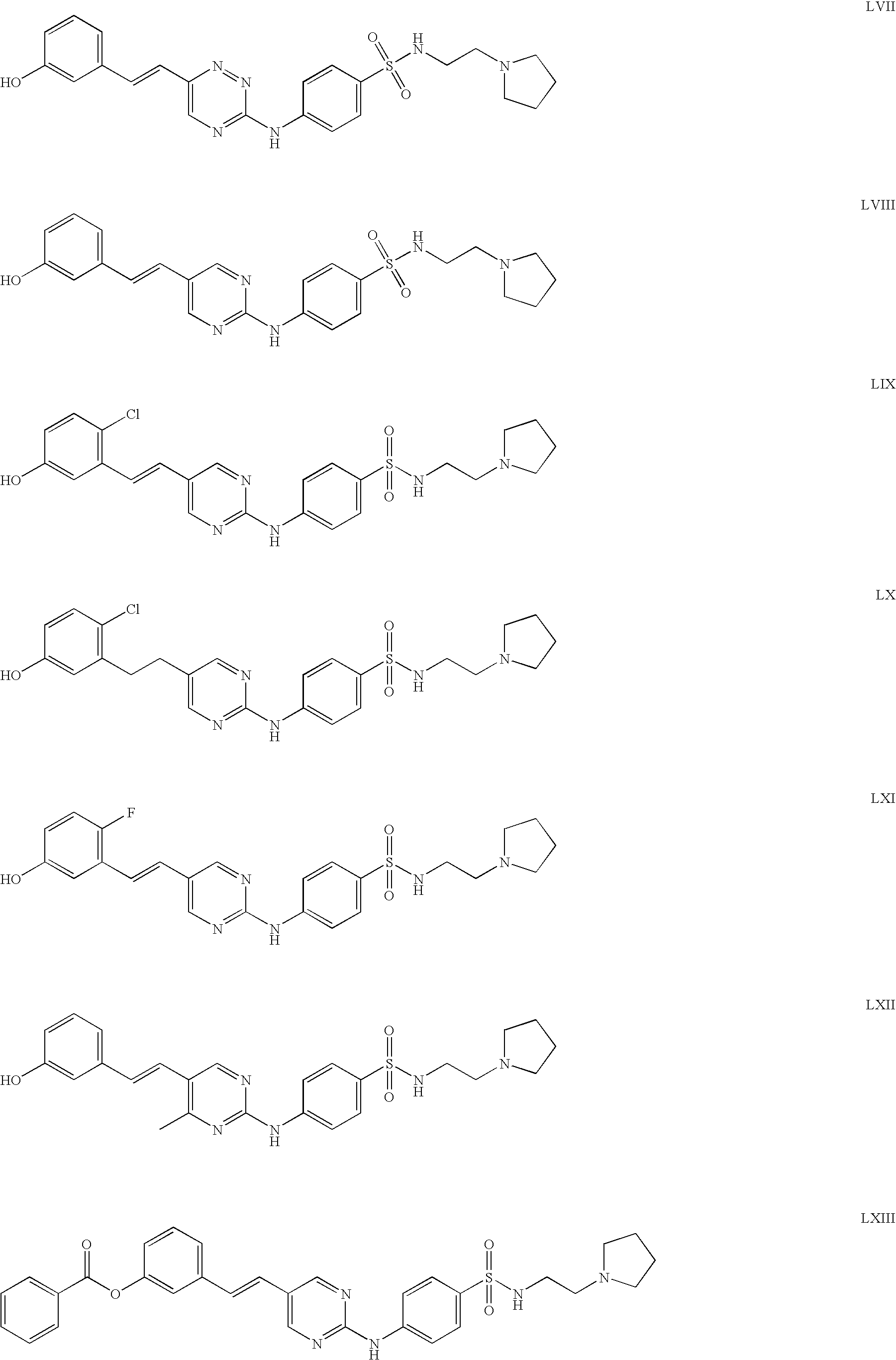

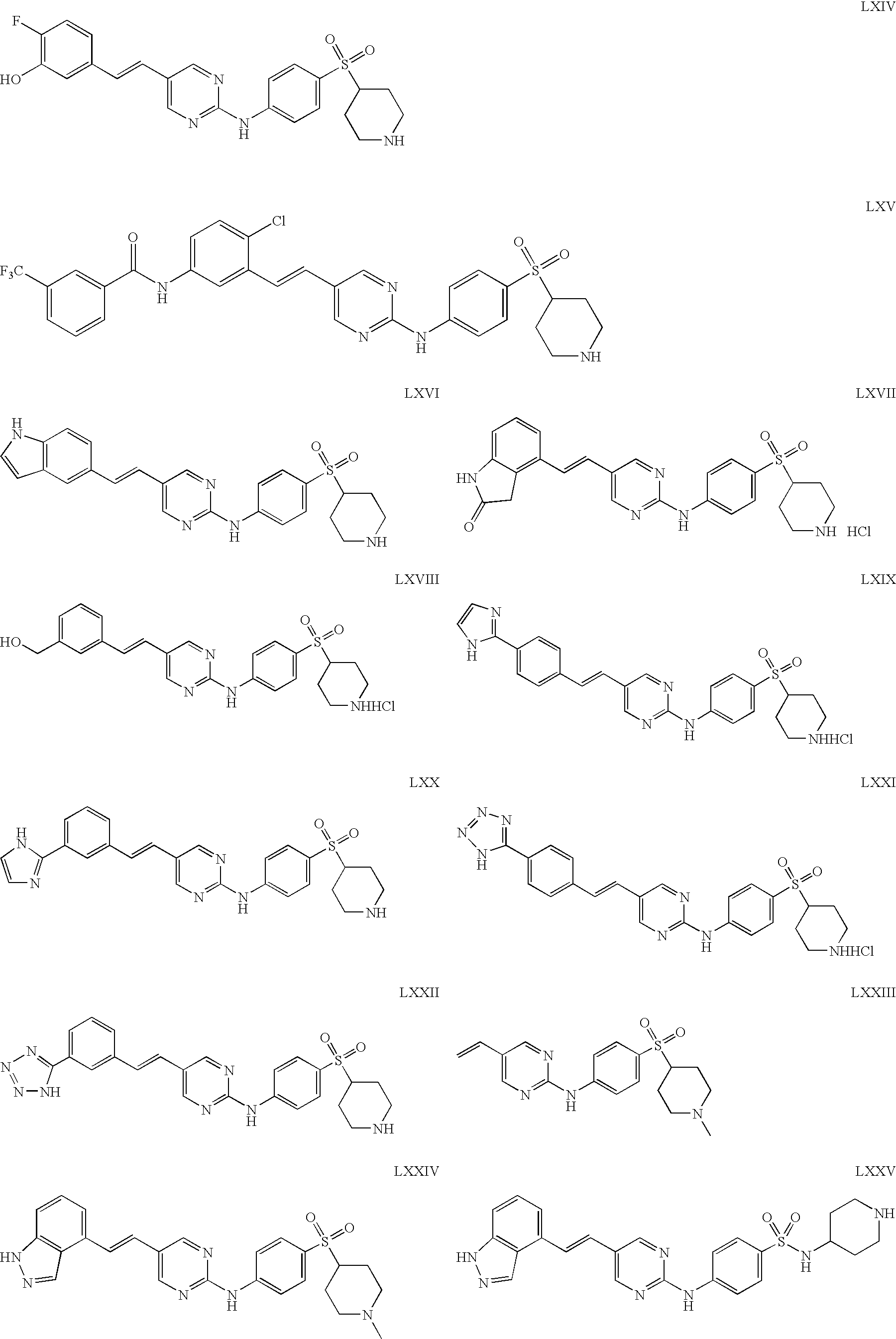

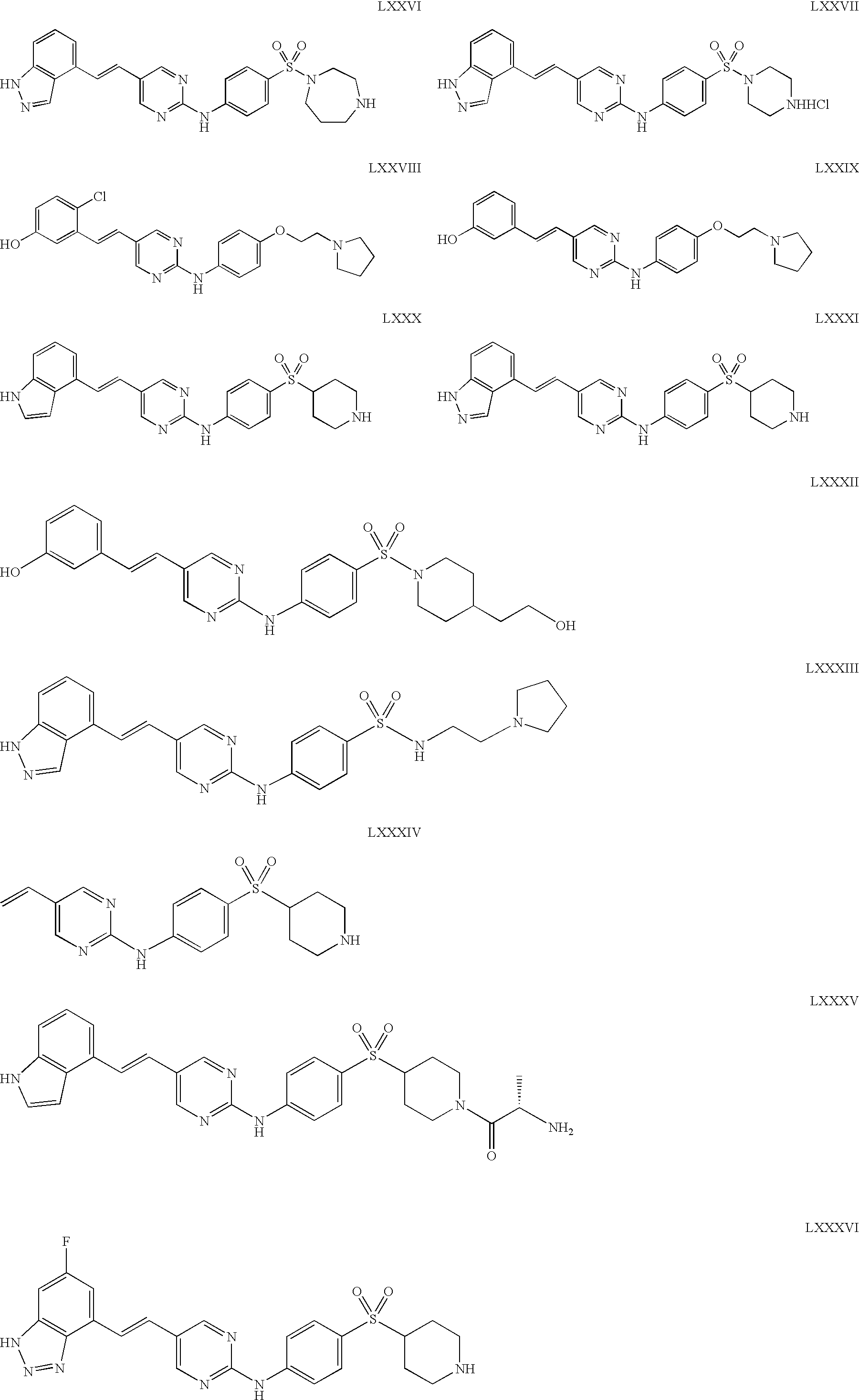

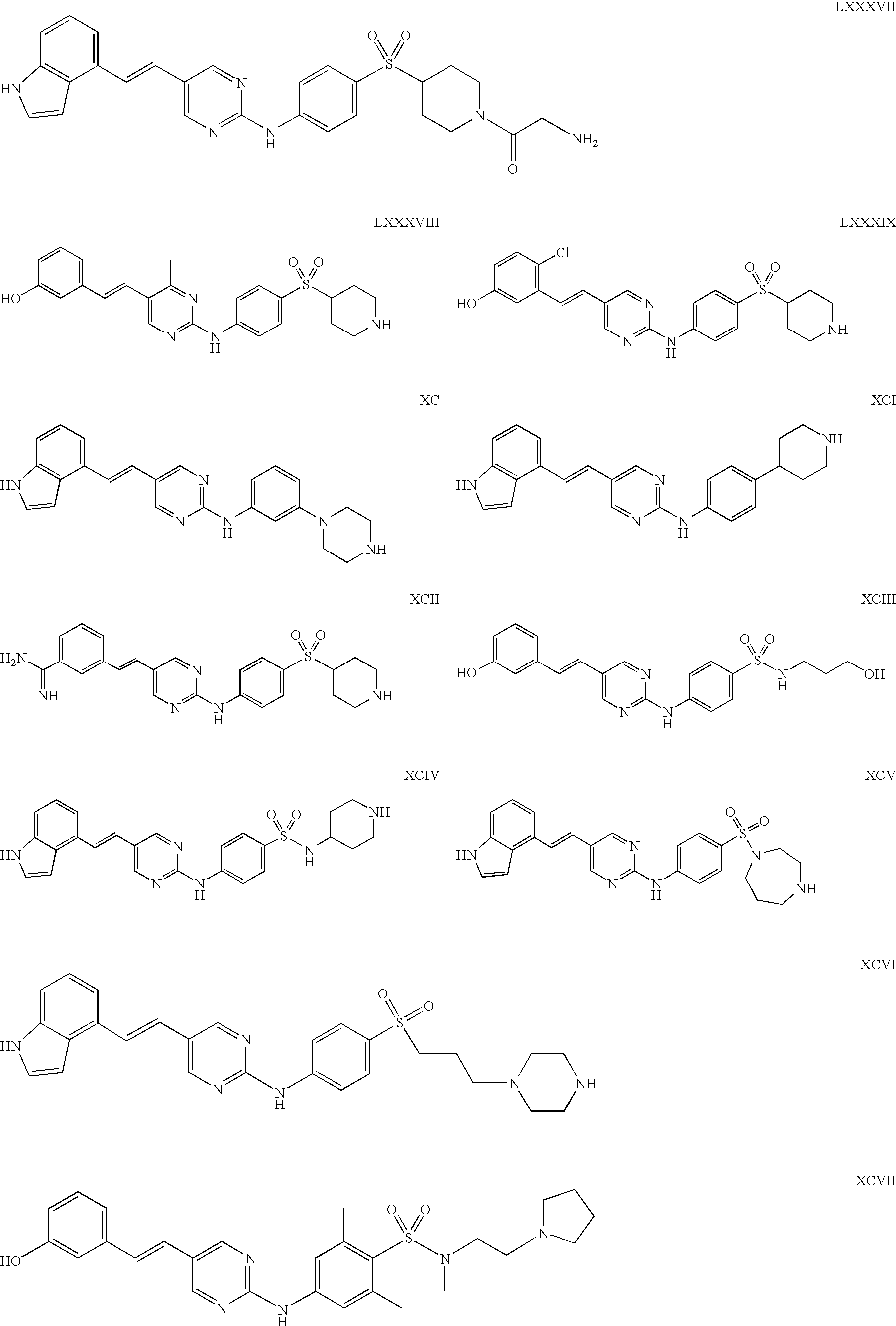

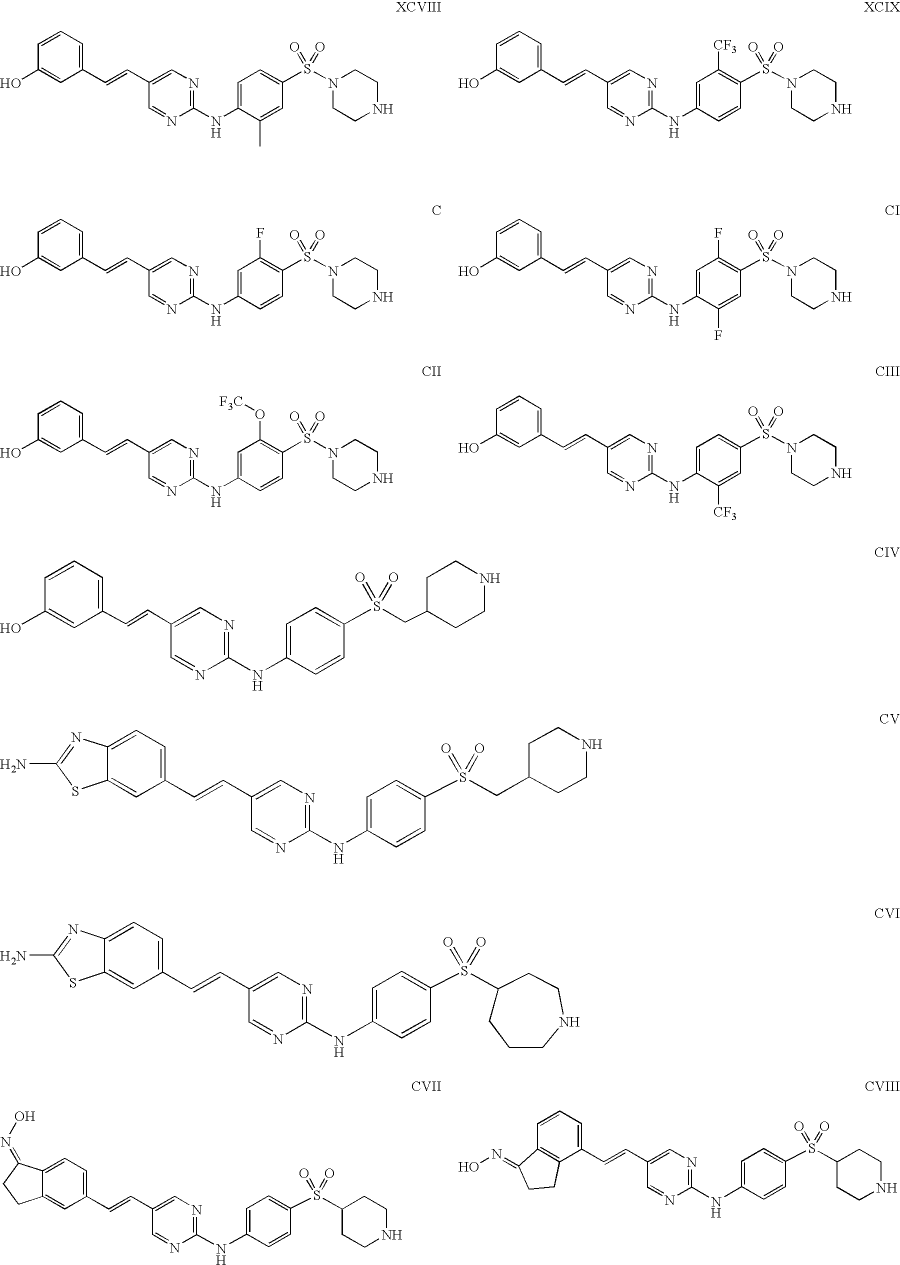

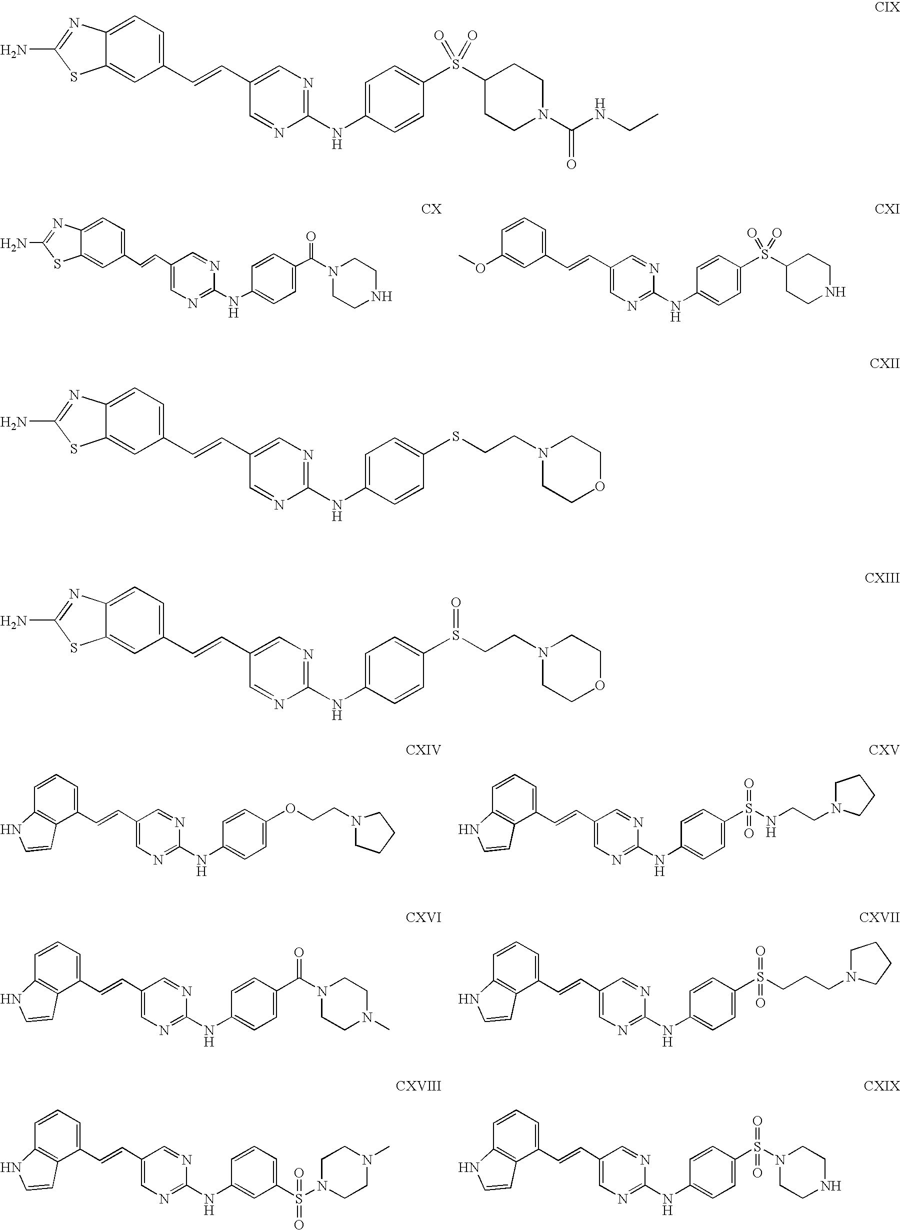

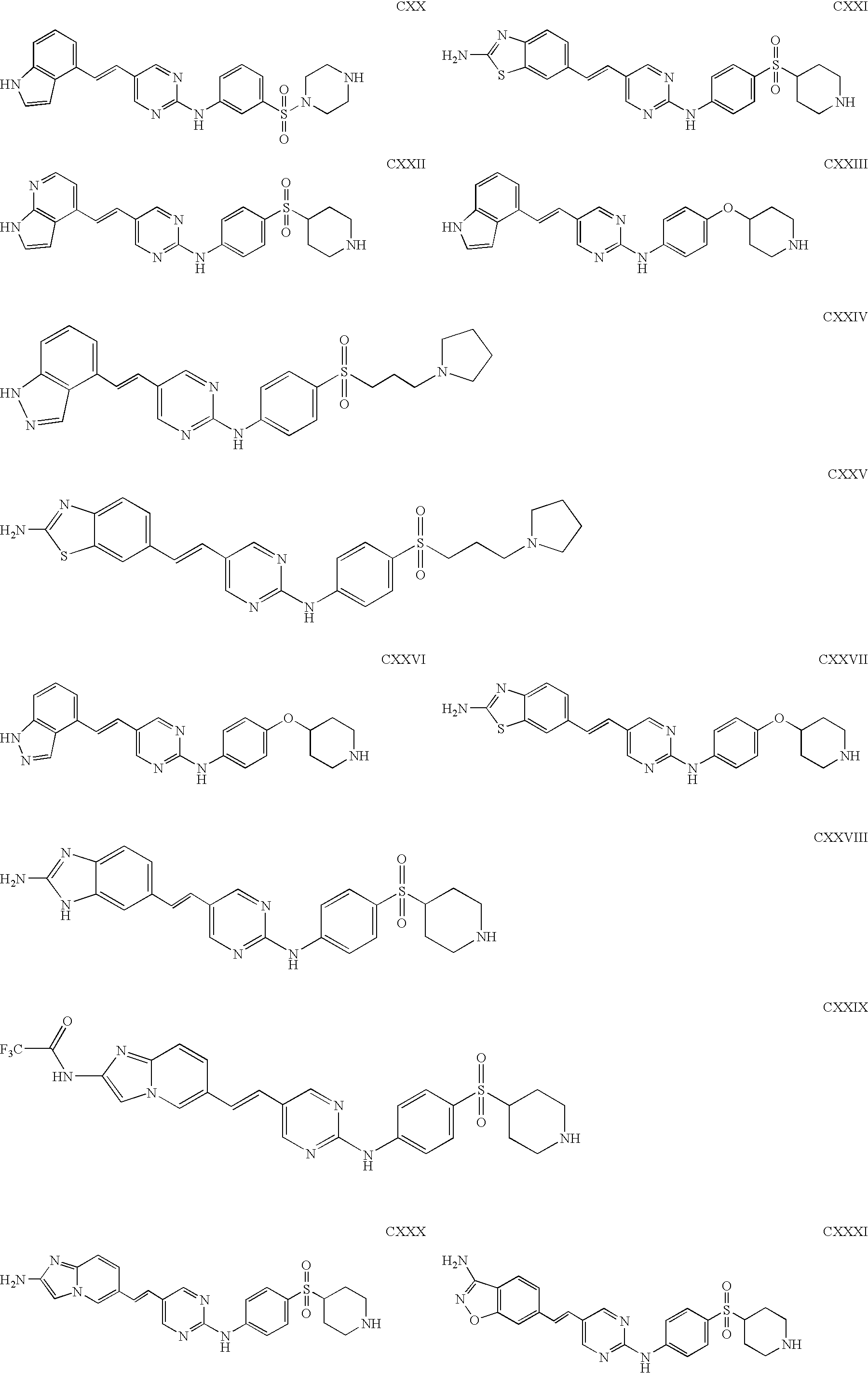

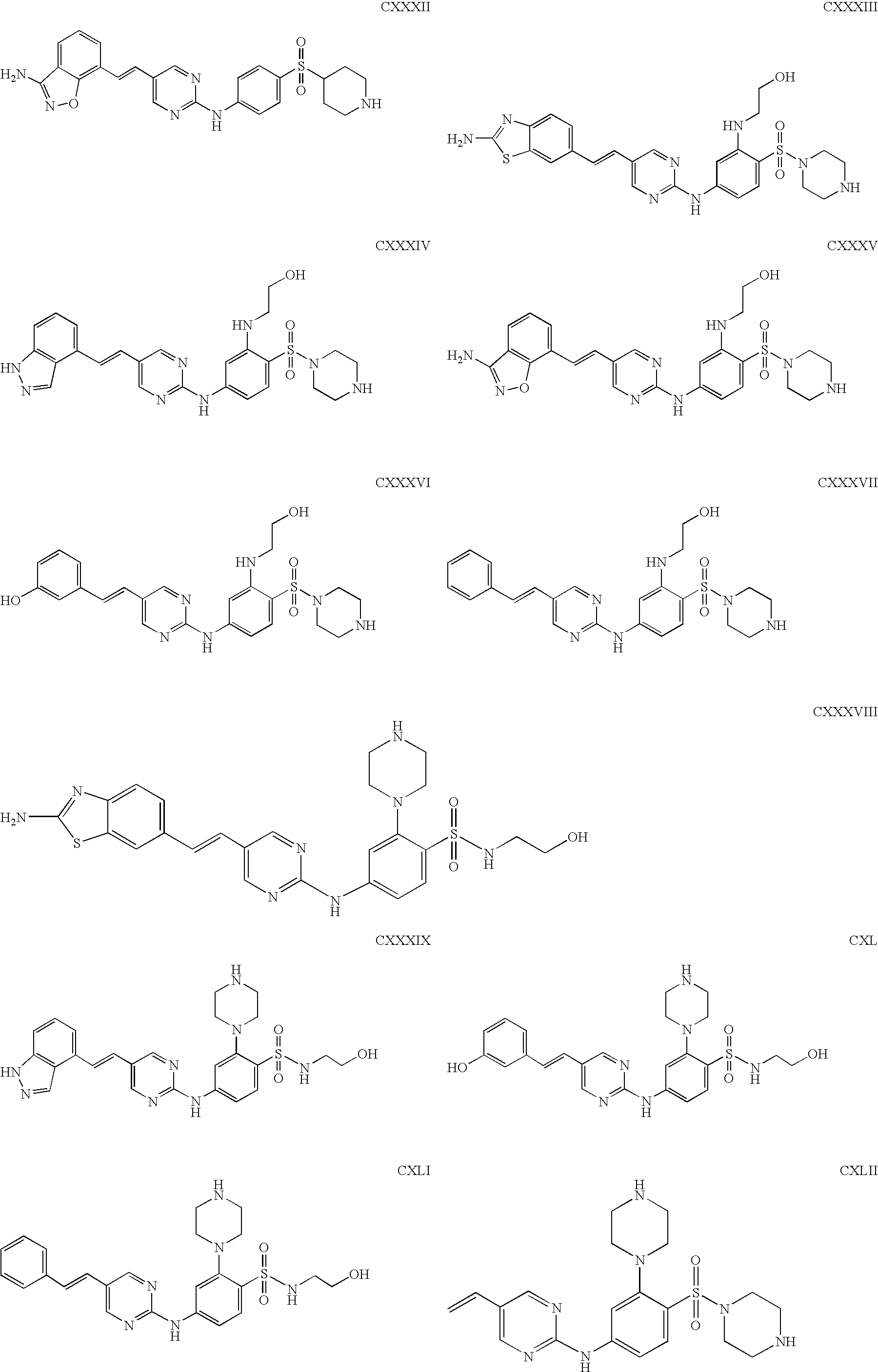

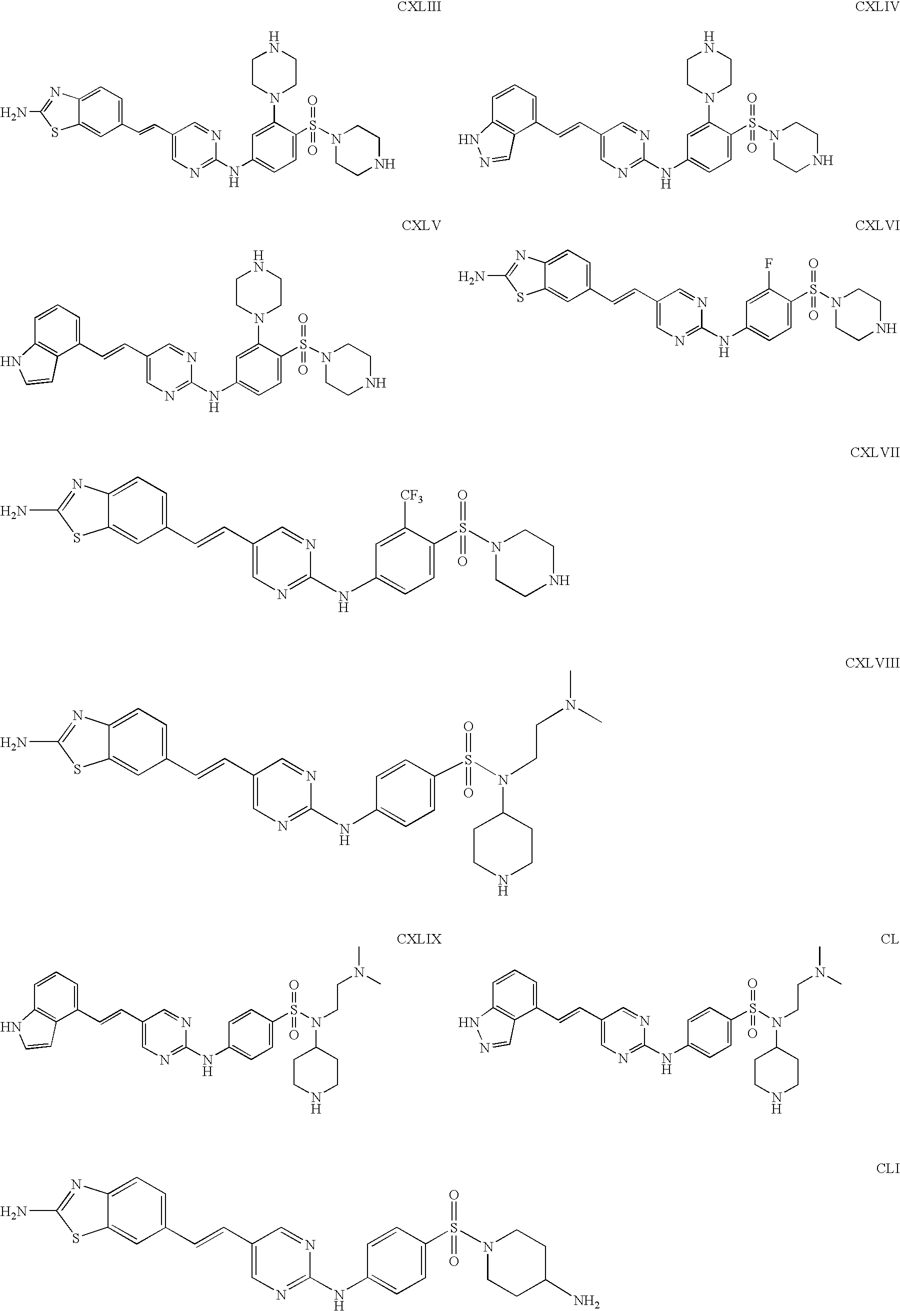

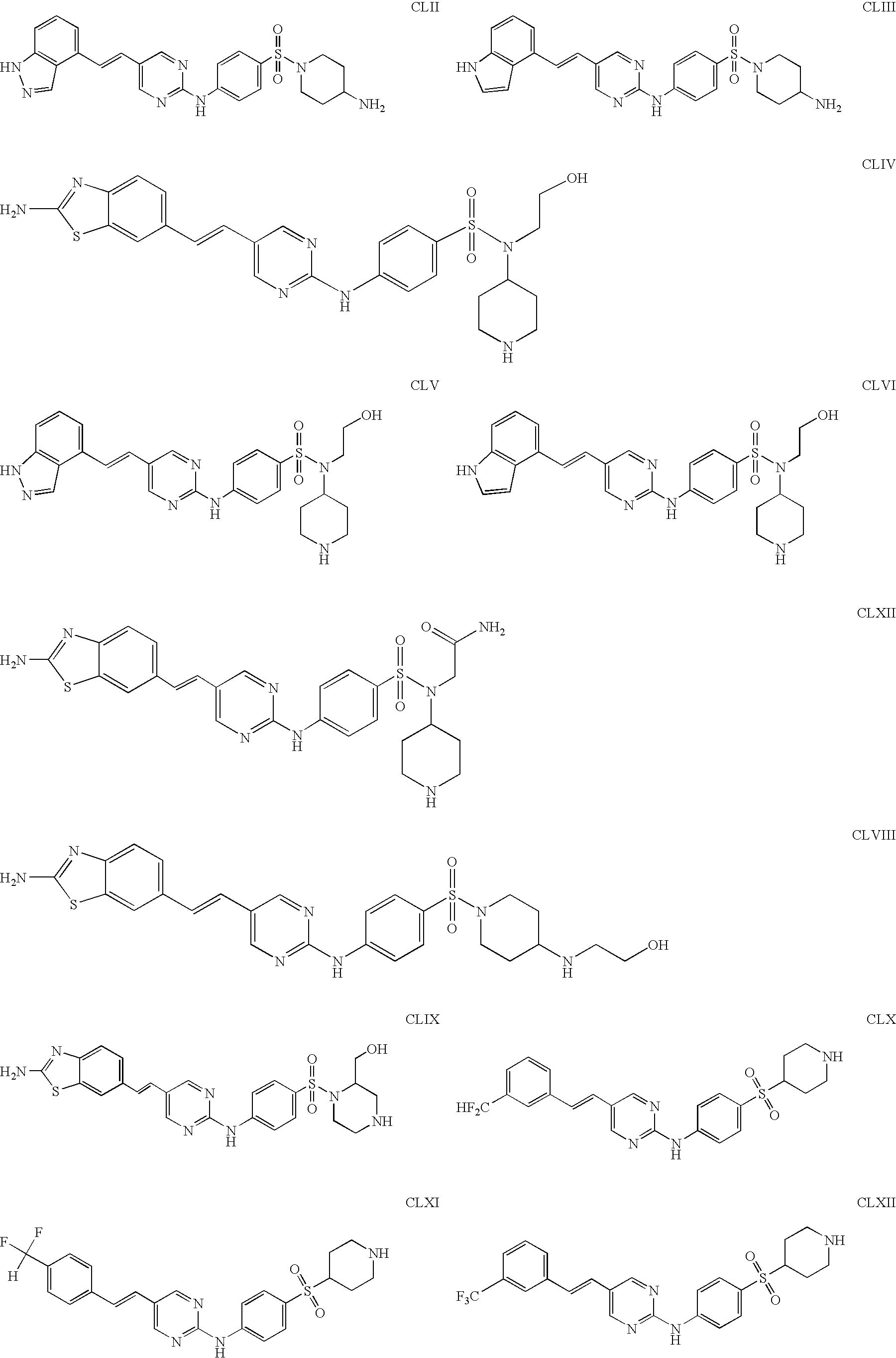

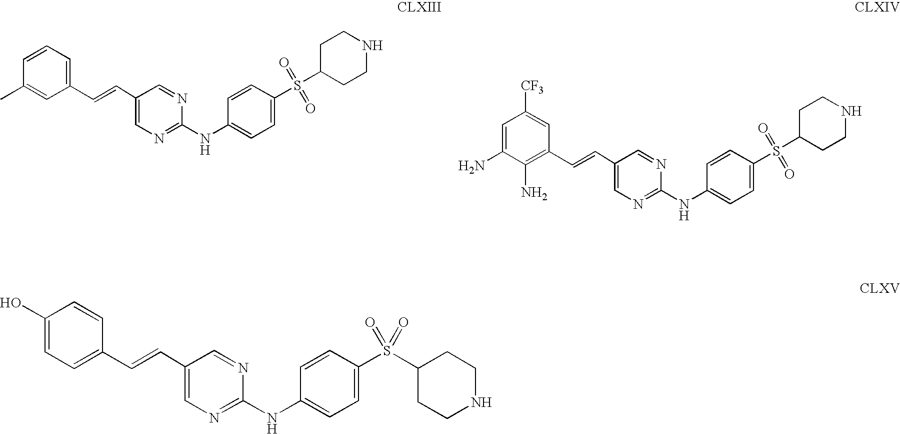

The compounds and methods of the present invention, or pharmaceutically acceptable salts, N-oxide(s), hydrates, solvates, crystal forms and individual diastereomers thereof, either when administered alone or in combination with other agents (e.g., chemotherapeutic agents or protein therapeutic agents described below) can be used for treating patients for whom traditional kinase-inhibition therapies with approved medication are inefficient. The medication is defined as “approved” if the medication is currently used for treatment of patients in need of treatment. Examples of such approved medications include compounds (B), (C), or (D) shown below. Compound (B) is also known under the trade name GLEEVEC and is available from Novartis, and compound (C) is known by the trade name TASIGNA, and is available from Novartis, and (D) is known by the tradename SPRYCEL and is available from Bristol Myers Squibb.

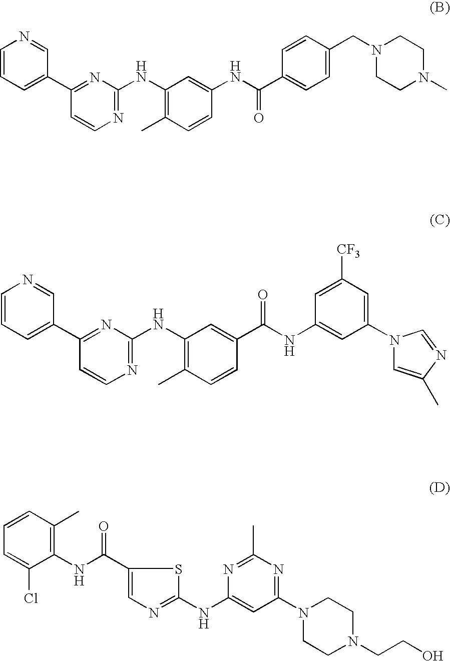

The inefficiency of the traditional kinase-inhibition treatments using compounds (B), (C), or (D) can be attributed to resistance the patients often develop to the treatment with these compound. The resistance can be caused by the kinase mutation, particularly the gatekeeper residue mutation. After the resistance has been developed, the traditional treatments (e.g., a GLEEVEC treatment of chronic myelogenous leukemia) no longer bring about sufficient therapeutic benefits. Therapy using a compound of the general structure (A) to replace all or a portion of the compounds (B), (C), or (D) can overcome the resistance and provide effective treatment.

Examples of disorders for treatment of which the compounds of structure (A), or pharmaceutically acceptable salts, N-oxide(s), hydrates, solvates, crystal forms and individual diastereomers thereof, can be used include, but are not limited to myeloproliferative disorders, proliferative diabetic retinopathy and other angiogenic-associated disorders including solid tumors and other types of cancer, eye disease, inflammation, psoriasis, and a viral infection. The kinds of cancer that can be treated include, but are not limited to, an alimentary/gastrointestinal tract cancer, colon cancer, liver cancer, skin cancer, breast cancer, ovarian cancer, prostate cancer, lymphoma, leukemia (including acute myelogenous leukemia and chronic myelogenous leukemia), kidney cancer, lung cancer, muscle cancer, bone cancer, bladder cancer or brain cancer.

Some examples of the diseases and disorders that can be treated also include ocular neovasculariaztion, infantile haemangiomas; organ hypoxia, vascular hyperplasia, organ transplant rejection, lupus, multiple sclerosis, rheumatoid arthritis, psoriasis, Type 1 diabetes and complications from diabetes, inflammatory disease, acute pancreatitis, chronic pancreatitis, asthma, allergies, adult respiratory distress syndrome, cardiovascular disease, liver disease, other blood disorders, asthma, rhinitis, atopic, dermatitits, autoimmune thryroid disorders, ulerative colitis, Crohn's disease, metastatic melanoma, Kaposi's sarcoma, multiple myeloma, conditions associated with cytokines, and other autoimmune diseases including glomerulonephritis, scleroderma, chronic thyroiditis, Graves' disease, autoimmune gastritis, autoimmune hemolytic anemia, autoimmune neutropenia, thrombocytopenia, atopy (e.g., allergic asthma, atopic dermatitis, or allergic rhinitis), chronic active hepatitis, myasthenia graivs, multiple sclerosis, inflammatory bowel disease, graft vs host disease, neurodegenerative diseases including motor neuron disease, Alzheimer's disease, Parkinson's disease, amyotrophic lateral scelerosis, Huntington's disease, cerebral ischemia, or neurodegenerative disease caused by traumatic injury, strike, gluatamate neurtoxicity or hypoxia; ischemic/reperfusion injury in stroke, myocardial ischemica, renal ischemia, heart attacks, cardiac hypertrophy, atherosclerosis and arteriosclerosis, organ hyoxia, and platelet aggregation.

Examples of some additional diseases and disorders that can be treated also include cell mediated hypersensitivity (allergic contact dermatitis, hypersensitivity pneumonitis), rheumatic diseases (e.g., systemic lupus erythematosus (SLE), juvenile arthritis, Sjogren's Syndrome, scleroderma, polymyositis, ankylosing spondylitis, psoriatic arthritis), viral diseases (Epstein Barr Virus, Hepatitis B, Hepatitis C, HIV, HTLV1, Vaicella-Zoster Virus, Human Papilloma Virus), food allergy, cutaneous inflammation, and immune suppression induced by solid tumors.

Embodiments of the present invention also provide articles of manufacture that can include a packaging material and a pharmaceutical composition contained within the packaging material. The packaging material can comprise a label which indicates that the pharmaceutical composition can be used for treatment of one or more disorders identified above.

The pharmaceutical composition can include a compound according to the present invention. In addition to a compound of the present invention, the pharmaceutical may also contain other therapeutic agents, and may be formulated, for example, by employing conventional solid or liquid vehicles or diluents, as well as pharmaceutical additives of a type appropriate to the mode of desired administration (for example, excipients, binders, preservatives, stabilizers, flavors, etc.) according to techniques known in the art of pharmaceutical formulation.

Thus, in one embodiment, the invention provides a pharmaceutical composition including a therapeutic agent and a compound of the invention. The compound is present in a concentration effective to treat, for example, cancer or to treat another disease or disorder described above.

The compounds of the invention may be formulated into therapeutic compositions as natural or salt forms. Pharmaceutically acceptable non-toxic salts include the base addition salts (formed with free carboxyl or other anionic groups) which may be derived from inorganic bases such as, for example, sodium, potassium, ammonium, calcium, or ferric hydroxides, and such organic bases as isopropylamine, trimethylamine, 2-ethylamino-ethanol, histidine, procaine, and the like. Such salts may also be formed as acid addition salts with any free cationic groups and will generally be formed with inorganic acids such as, for example, hydrochloric, sulfuric, or phosphoric acids, or organic acids such as acetic, citric, p-toluenesulfonic, methanesulfonic acid, oxalic, tartaric, mandelic, and the like.

Salts of the invention can include amine salts formed by the protonation of an amino group with inorganic acids such as hydrochloric acid, hydrobromic acid, hydroiodic acid, sulfuric acid, phosphoric acid, and the like. Salts of the invention can also include amine salts formed by the protonation of an amino group with suitable organic acids, such as p-toluenesulfonic acid, acetic acid, methanesulfonic acid and the like. Additional excipients which are contemplated for use in the practice of the present invention are those available to those of ordinary skill in the art, for example, those found in the United States Pharmacopeia Vol. XXII and National Formulary Vol. XVII, U.S. Pharmacopeia Convention, Inc., Rockville, Md. (1989), the relevant contents of which is incorporated herein by reference. In addition, polymorphs of the invention compounds are included in the present invention.

Pharmaceutical compositions of the invention may be administered by any suitable means, for example, orally, such as in the form of tablets, capsules, granules or powders; sublingually; buccally; parenterally, such as by subcutaneous, intravenous, intramuscular, intrathecal, or intracisternal injection or infusion techniques (e.g., as sterile injectable aqueous or non-aqueous solutions or suspensions); nasally such as by inhalation spray; topically, such as in the form of a cream or ointment; or rectally such as in the form of suppositories; in dosage unit formulations containing non-toxic, pharmaceutically acceptable vehicles or diluents. The present compounds may, for example, be administered in a form suitable for immediate release or extended release. Immediate release or extended release may be achieved by the use of suitable pharmaceutical compositions comprising the present compounds, or, particularly in the case of extended release, by the use of devices such as subcutaneous implants or osmotic pumps. The present compounds may also be administered liposomally.

In addition to primates, such as humans, a variety of other mammals can be treated according to the method of the present invention. For instance, mammals including, but not limited to, cows, sheep, goats, horses, dogs, cats, guinea pigs, rats or other bovine, ovine, equine, canine, feline, rodent or murine species can be treated. However, the method can also be practiced in other species, such as avian species (e.g., chickens).

The pharmaceutical compositions for the administration of the compounds of this embodiment, either alone or in combination with other therapeutic agents, may conveniently be presented in dosage unit form and may be prepared by any of the methods well known in the art of pharmacy. All methods include bringing the active ingredient into association with the carrier which constitutes one or more accessory ingredients. In general, the pharmaceutical compositions are prepared by uniformly and intimately bringing the active ingredient into association with a liquid carrier or a finely divided solid carrier or both, and then, if necessary, shaping the product into the desired formulation. In the pharmaceutical composition the active object compound is included in an amount sufficient to produce the desired effect upon the process or condition of diseases. The pharmaceutical compositions containing the active ingredient may be in a form suitable for oral use, for example, as tablets, troches, lozenges, aqueous or oily suspensions, dispersible powders or granules, emulsions, hard or soft capsules, or syrups or elixirs.

Compositions intended for oral use may be prepared according to any method known to the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed. They may also be coated to form osmotic therapeutic tablets for control release.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin, or olive oil.

Aqueous suspensions contain the active materials in admixture with excipients suitable for the manufacture of aqueous suspensions. Such excipients are suspending agents, for example sodium carboxymethylcellulose, methylcellulose, hydroxy-propylmethylcellulose, sodium alginate, polyvinyl-pyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. Also useful as a solubilizer is polyethylene glycol, for example. The aqueous suspensions may also contain one or more preservatives, for example ethyl, or n-propyl, p-hydroxybenzoate, one or more coloring agents, one or more flavoring agents, and one or more sweetening agents, such as sucrose or saccharin.

Oily suspensions may be formulated by suspending the active ingredient in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present.

Syrups and elixirs may be formulated with sweetening agents, for example glycerol, propylene glycol, sorbitol or sucrose. Such formulations may also contain a demulcent, a preservative and flavoring and coloring agents.

The pharmaceutical compositions may be in the form of a sterile injectable aqueous or oleagenous suspension. This suspension may be formulated according to the known art using those suitable dispersing or wetting agents and suspending agents which have been mentioned above. The sterile injectable preparation may also be a sterile injectable solution or suspension in a parenterally-acceptable diluent or solvent or cosolvent or complexing agent or dispersing agent or excipient or combination thereof, for example 1,3-butanediol, polyethylene glycols, polypropylene glycols, ethanol or other alcohols, povidones, various brands of TWEEN surfactant, sodium dodecyl sulfate, sodium deoxycholate, dimethylacetamide, polysorbates, poloxamers, cyclodextrins, lipids, and excipients such as inorganic salts (e.g., sodium chloride), buffering agents (e.g., sodium citrate, sodium phosphate), and sugars (e.g., saccharose and dextrose). Among the acceptable vehicles and solvents that may be employed are water, dextrose solutions, Ringer's solutions and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

Depending on the condition being treated, these pharmaceutical compositions may be formulated and administered systemically or locally. Techniques for formulation and administration may be found in the latest edition of “Remington's Pharmaceutical Sciences” (Mack Publishing Co, Easton Pa.). Suitable routes may, for example, include oral or transmucosal administration; as well as parenteral delivery, including intramuscular, subcutaneous, intramedullary, intrathecal, intraventricular, intravenous, intraperitoneal, or intranasal administration. For injection, the pharmaceutical compositions of the invention may be formulated in aqueous solutions, preferably in physiologically compatible buffers such as Hanks' solution, Ringer's solution, or physiologically buffered saline. For tissue or cellular administration, penetrants appropriate to the particular barrier to be permeated are used in the formulation. Such penetrants are generally known in the art. Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances that increase the viscosity of the suspension, such as sodium carboxymethyl cellulose, sorbitol, or dextran. Optionally, the suspension may also contain suitable stabilizers or agents that increase the solubility of the compounds to allow for the preparation of highly concentrated solutions.

The compounds of the present invention may also be administered in the form of suppositories for rectal administration of the drug. These compositions can be prepared by mixing the drug with a suitable non-irritating excipient which is solid at ordinary temperatures but liquid at the rectal temperature and will therefore melt in the rectum to release the drug. Such materials are cocoa butter and polyethylene glycols.

For topical use, creams, ointments, jellies, solutions or suspensions, etc., containing the compounds of the present invention are employed. (For purposes of this application, topical application shall include mouthwashes and gargles).

In one embodiment, the invention compounds are administered in combination with an anti-inflammatory agent, antihistamines, chemotherapeutic agent, immunomodulator, therapeutic antibody or a protein kinase inhibitor, e.g., a tyrosine kinase inhibitor, to a subject in need of such treatment. While not wanting to be limiting, chemotherapeutic agents include antimetabolites, such as methotrexate, DNA cross-linking agents, such as cisplatin/carboplatin; alkylating agents, such as canbusil; topoisomerase I inhibitors such as dactinomicin; microtubule inhibitors such as taxol (paclitaxol), and the like. Other chemotherapeutic agents include, for example, a vinca alkaloid, mitomycin-type antibiotic, bleomycin-type antibiotic, antifolate, colchicine, demecoline, etoposide, taxane, anthracycline antibiotic, doxorubicin, daunorubicin, carminomycin, epirubicin, idarubicin, mithoxanthrone, 4-dimethoxy-daunomycin, 11-deoxydaunorubicin, 13-deoxydaunorubicin, adriamycin-14-benzoate, adriamycin-14-octanoate, adriamycin-14-naphthaleneacetate, amsacrine, carmustine, cyclophosphamide, cytarabine, etoposide, lovastatin, melphalan, topetecan, oxalaplatin, chlorambucil, methtrexate, lomustine, thioguanine, asparaginase, vinblastine, vindesine, tamoxifen, or mechlorethamine. While not wanting to be limiting, therapeutic antibodies include antibodies directed against the HER2 protein, such as trastuzumab; antibodies directed against growth factors or growth factor receptors, such as bevacizumab, which targets vascular endothelial growth factor, and OSI-774, which targets epidermal growth factor; antibodies targeting integrin receptors, such as Vitaxin (also known as MEDI-522), and the like. Classes of anticancer agents suitable for use in compositions and methods of the present invention include, but are not limited to: 1) alkaloids, including, microtubule inhibitors (e.g., Vincristine, Vinblastine, and Vindesine, etc.), microtubule stabilizers (e.g., Paclitaxel [Taxol], and Docetaxel, Taxotere, etc.), and chromatin function inhibitors, including, topoisomerase inhibitors, such as, epipodophyllotoxins (e.g., Etoposide [VP-16], and Teniposide [VM-26], etc.), and agents that target topoisomerase I (e.g., Camptothecin and Isirinotecan [CPT-11], etc.); 2) covalent DNA-binding agents [alkylating agents], including, nitrogen mustards (e.g., Mechlorethamine, Chlorambucil, Cyclophosphamide, Ifosphamide, and Busulfan [Myleran], etc.), nitrosoureas (e.g., Carmustine, Lomustine, and Semustine, etc.), and other alkylating agents (e.g., Dacarbazine, Hydroxymethylmelamine, Thiotepa, and Mitocycin, etc.); 3) noncovalent DNA-binding agents [antitumor antibiotics], including, nucleic acid inhibitors (e.g., Dactinomycin [Actinomycin D], etc.), anthracyclines (e.g., Daunorubicin [Daunomycin, and Cerubidine], Doxorubicin [Adriamycin], and Idarubicin [Idamycin], etc.), anthracenediones (e.g., anthracycline analogues, such as, [Mitoxantrone], etc.), bleomycins (Blenoxane), etc., and plicamycin (Mithramycin), etc.; 4) antimetabolites, including, antifolates (e.g., Methotrexate, Folex, and Mexate, etc.), purine antimetabolites (e.g., 6-Mercaptopurine [6-MP, Purinethol], 6-Thioguanine [6-TG], Azathioprine, Acyclovir, Ganciclovir, Chlorodeoxyadenosine, 2-Chlorodeoxyadenosine [CdA], and 2′-Deoxycoformycin [Pentostatin], etc.), pyrimidine antagonists (e.g., fluoropyrimidines [e.g., 5-fluorouracil (Adrucil), 5-fluorodeoxyuridine (FdUrd) (Floxuridine)] etc.), and cytosine arabinosides (e.g., Cytosar [ara-C] and Fludarabine, etc.); 5) enzymes, including, L-asparaginase; 6) hormones, including, glucocorticoids, such as, antiestrogens (e.g., Tamoxifen, etc.), nonsteroidal antiandrogens (e.g., Flutamide, etc.), and aromatase inhibitors (e.g., anastrozole [Arimidex], etc.); 7) platinum compounds (e.g., Cisplatin and Carboplatin, etc.); 8) monoclonal antibodies conjugated with anticancer drugs, toxins, and/or radionuclides, etc.; 9) biological response modifiers (e.g., interferons [e.g., IFN-.alpha., etc.] and interleukins [e.g., IL-2, etc.], etc.); 10) adoptive immunotherapy; 11) hematopoietic growth factors; 12) agents that induce tumor cell differentiation (e.g., all-trans-retinoic acid, etc.); 13) gene therapy techniques; 14) antisense therapy techniques; 15) tumor vaccines; 16) therapies directed against tumor metastases (e.g., Batimistat, etc.); and 17) inhibitors of angiogenesis.

The pharmaceutical composition and method of the present invention may further comprise other therapeutically active compounds as noted herein which are usually applied in the treatment of the above mentioned pathological conditions. Examples of other therapeutic agents include the following: cyclosporins (e.g., cyclosporin A), CTLA4-Ig, antibodies such as ICAM-3, anti-IL-2 receptor (Anti-Tac), anti-CD45RB, anti-CD2, anti-CD3 (OKT-3), anti-CD4, anti-CD80, anti-CD86, agents blocking the interaction between CD40 and gp39, such as antibodies specific for CD40 and/or gp39 (i.e., CD154), fusion proteins constructed from CD40 and gp39 (CD40Ig and CD8gp39), inhibitors, such as nuclear translocation inhibitors, of NF-kappa B function, such as deoxyspergualin (DSG), cholesterol biosynthesis inhibitors such as HMG CoA reductase inhibitors (lovastatin and simvastatin), non-steroidal antiinflammatory drugs (NSAIDs) such as ibuprofen and cyclooxygenase inhibitors such as rofecoxib, steroids such as prednisone or dexamethasone, gold compounds, antiproliferative agents such as methotrexate, FK506 (tacrolimus, Prograf), mycophenolate mofetil, cytotoxic drugs such as azathioprine and cyclophosphamide, TNF-a inhibitors such as tenidap, anti-TNF antibodies or soluble TNF receptor, and rapamycin (sirolimus or Rapamune) or derivatives thereof.

Other agents that may be administered in combination with invention compounds include protein therapeutic agents such as cytokines, immunomodulatory agents and antibodies. As used herein the term “cytokine” encompasses chemokines, interleukins, lymphokines, monokines, colony stimulating factors, and receptor associated proteins, and functional fragments thereof. As used herein, the term “functional fragment” refers to a polypeptide or peptide which possesses biological function or activity that is identified through a defined functional assay.

The cytokines include endothelial monocyte activating polypeptide II (EMAP-II), granulocyte-macrophage-CSF (GM-CSF), granulocyte-CSF (G-CSF), macrophage-CSF (M-CSF), IL-1, IL-2, IL-3, IL-4, IL-5, IL-6, IL-12, and IL-13, interferons, and the like and which is associated with a particular biologic, morphologic, or phenotypic alteration in a cell or cell mechanism.

When other therapeutic agents are employed in combination with the compounds of the present invention they may be used for example in amounts as noted in the Physician Desk Reference (PDR) or as otherwise determined by one having ordinary skill in the art.

In the treatment or prevention of conditions which involve cellular proliferation, an appropriate dosage level can generally be between about 0.01 and about 1000 mg per 1 kg of patient body weight per day which can be administered in single or multiple doses. For example, the dosage level can be between about 0.01 and about 250 mg/kg per day; more narrowly, between about 0.5 and about 100 mg/kg per day. A suitable dosage level can be between about 0.01 and about 250 mg/kg per day, between about 0.05 and about 100 mg/kg per day, or between about 0.1 and about 50 mg/kg per day, or about 1.0 mg/kg per day. For example, within this range the dosage can be between about 0.05 and about 0.5 mg/kg per day, or between about 0.5 and about 5 mg/kg per day, or between about 5 and about 50 mg/kg per day. For oral administration, the compositions can be provided in the form of tablets containing between about 1.0 and about 1,000 mg of the active ingredient, for example, about 1.0, about 5.0, about 10.0, about 15.0, about 20.0, about 25.0, about 50.0, about 75.0, about 100.0, about 150.0, about 200.0, about 250.0, about 300.0, about 400.0, about 500.0, about 600.0, about 750.0, about 800.0, about 900.0, and about 1,000.0 mg of the active ingredient for the symptomatic adjustment of the dosage to the patient to be treated. The compounds can be administered on a regimen of 1 to 4 times per day, such as once or twice per day. There may be a period of no administration followed by another regimen of administration.

It will be understood, however, that the specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

Compounds of the present invention can be used, alone or in combination with an effective amount of a therapeutic antibody (or therapeutic fragment thereof), a chemotherapeutic or an immunotoxic agent, for treatment of tumors. Illustrative examples of chemotherapeutic agents that can be used for this purpose include doxorubicin, docetaxel, or taxol. It should be further understood that the invention includes combination therapy including a compound of the invention, including but not limited to vasculostatic agents, such as tyrosine, serine or threonine kinase inhibitors, for example, and any chemotherapeutic agent or therapeutic antibody.

### C. EXAMPLES

The following examples are provided to further illustrate the advantages and features of the present invention, but are not intended to limit the scope of the invention.

### Example 1

### General Methods

All experiments were performed under anhydrous conditions (i.e. dry solvents) in an atmosphere of argon, except where stated, using oven-dried apparatus and employing standard techniques in handling air-sensitive materials. Aqueous solutions of sodium bicarbonate (NaHCO₃) and sodium chloride (brine) were saturated. Analytical thin layer chromatography (TLC) was carried out on Merck Kieselgel 60 F₂₅₄ plates with visualization by ultraviolet and/or anisaldehyde, potassium permanganate or phosphomolybdic acid dips. Reverse-phase HPLC chromatography was carried out on Gilson 215 liquid handler equipped with Waters SymmetryShield™ RP18 7 μm (40×100 mm) Prep-Pak cartridge. Mobile phase consisted of standard acetonitrile (ACN) and DI Water, each with 0.1% TFA added. Purification was carried out at a flow rate of 40 mL/min. NMR spectra: ¹H Nuclear magnetic resonance spectra were recorded at 500 MHz. Data are presented as follows: chemical shift, multiplicity (s=singlet, d=doublet, t=triplet, q=quartet, qn=quintet, dd=doublet of doublets, m=multiplet, br s=broad singlet), coupling constant (J/Hz) and integration. Coupling constants were taken directly from the spectra and are uncorrected. Low resolution mass spectra: Electrospray (ES+) ionization was used. The protonated parent ion (M+H) or fragment of highest mass is quoted. Analytical gradient consisted of 10% ACN in water ramping up to 100% ACN over 5 minutes unless otherwise stated.

### Example 2

### 5-Vinyl-pyrimidin-2-ylamine (Intermediate 1)

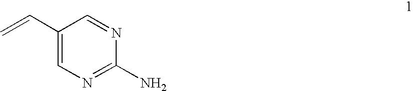

To a suspension of 5-bromro-pyrimidin-2-ylamine (327 g, 1.89 mol) in DMF (3 L) was added KOAc (250 g, 2.55 mol) and Pd(PPh₃)₄ (53 g, 45.9 mmol) under nitrogen. The reaction mixture was heated to 100° C. under ethane and stirred for 12 h. Upon completion, the reaction mixture was filtered. The filtrate was concentrated and purified by chromatography (petroleum:EtOAc=3:1) to afford crude product, which was re-crystallized from EtOH (500 ml) to afford intermediate 1 (50 g, 22%) as a white solid.

### Example 3

### 4-Hydroxy-piperidine-1-carboxylic acid tert-butyl ester (Intermediate 2)

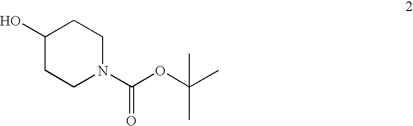

To a solution of piperidin-4-ol (153 g, 1.51 mol) and Et₃N (210 g, 2.08 mol) in MeOH (800 mL) was added dropwise a solution of di-tert-butyl dicarbonate (350 g, 1.60 mol) in MeOH (200 mL) under ice cooling. After the addition was complete, the resulting mixture was stirred at room temperature for 24 h. Upon completion, the reaction mixture was concentrated, and the residue was partitioned between 1N aqueous HCl solution (1000 mL) and EtOAc. The organic layer was dried over MgSO₄, and concentrated to give intermediate 2 (275 g, 90%).

### Example 4

### 4-Methanesulfonyloxy-piperidine-1-carboxylic acid tert-butyl ester (Intermediate 3)

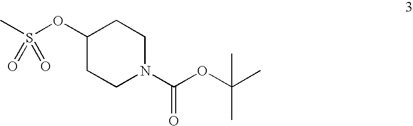

To a solution of intermediate 2 (200 g, 1.0 mol) and Et₃N (204 g, 2.0 mol) in CH₂Cl₂ (3000 mL) was added dropwise a solution of MsCl (130 g, 1.14 mol) in CH₂Cl₂ (500 mL) under ice cooling. After the addition completed, the resulting mixture was stirred at room temperature for 24 h. Upon completion, the reaction mixture was washed with water and a 1N aqueous HCl solution (1000 mL). The organic layer was dried over MgSO₄, and concentrated to give intermediate 3 (230 g, 82%) as a white solid.

### Example 5

### 4-(4-Bromo-phenylsulfanyl)-piperidine-1-carboxylic acid tert-butyl ester (Intermediate 4)

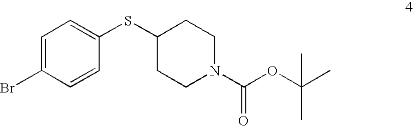

Intermediate 3 (82.0 g, 0.29 mol), 4-bromothiophenol (50.0 g, 0.27 mol) and K₂CO₃ (80.0 g, 0.58 mol) were mixed in CH₃CN (5000 mL) at room temperature. The mixture was heated to reflux and stirred for 24 h. Upon completion, the reaction mixture was filtered and the filtrate was concentrated. The residue was dissolved in EtOAc and washed with 1N NaOH. The organic layer was concentrated to give crude product, which was purified by column chromatography (silica, elute; petroleum:EtOAc=10:0, 10:1) to give intermediate 4 (62 g, 79%) as a white solid.

### Example 6

### 4-(4-Bromo-benzenesulfonyl)-piperidine-1-carboxylic acid tert-butyl ester (Intermediate 5)

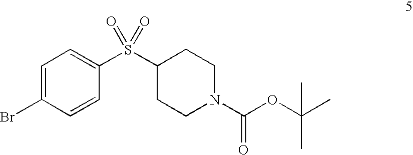

Water (40 mL) was added to alumina (140 g, 1.37 mol) at room temperature and stirred for 5 min. To the resulting slurry was added CHCl₃ (800 mL), a solution of intermediate 4 (62 g, 0.17 mol) in CHCl₃ (900 mL) and oxone (170 g, 0.28 mol) successively. The resulting slurry was heated to reflux and stirred for 24 h. Upon completion, the reaction mixture was cooled to room temperature and then filtered. The filtrate was washed with saturated aqueous Na₂SO₃ solution, dried over MgSO₄, and evaporated to give crude product, which was purified by re-crystallization from petroleum/EtOAc (2000 ml/500 ml) to give intermediate 5 (59 g, 87%) as a white solid.

### Example 7

### 4-[4-(5-Vinyl-pyrimidin-2-ylamino)-benzenesulfonyl]-piperidine-1-carboxylic acid tert-butyl ester (Intermediate 6)

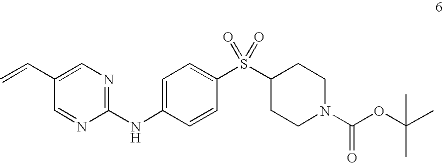

To a solution of intermediate 1 (25 g, 0.21 mol) and intermediate 5 (70 g, 0.17 mol) in dioxane (1500 mL) was added Pd₂(dba)₂ (9 g, 9.8 mmol), Cs₂CO₃ (130 g, 0.4 mol) and Xantphos (22 g, 0.04 mol) successively at room temperature. The resulting mixture heated to reflux and stirred for 12 h. Upon completion, the reaction mixture was filtered. The filtrate was concentrated and simply purified by chromatography (silica, elute; Petroleum:EtOAc=2:1) to give crude product, which was further purified via re-crystallization from EtOH (200 mL) to afford intermediate 6 (40 g, 52%) as a white solid.

### Example 8

### 5-(3-Nitrostyryl)pyrimidin-2-amine (Intermediate 7)

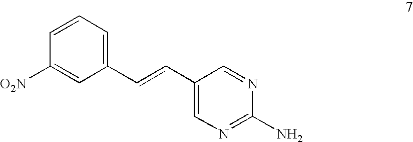

To a solution of 1-nitro-3-vinylbenzene (300 mg, 2 mmol) in DMF (10 mL) was added 5-bromopyrimidin-2-amine (348 mg, 2 mmol), Pd(OAc)₂ (90 mg, 0.4 mmol), and Et₃N (1.1 mL, 8 mmol). The reaction was heated at 150° C. for 15 min in microwave. The solid was filtered off and washed with EtOAc. The filtrate was washed with brine (1×100 mL). The organic solution was separated. The aqueous was extracted with EtOAc (2×10 mL). The combined organic phase was dried (Na₂SO₄) and concentrated until 5 mL remaining. The hexanes (100 mL) were added to above solution and the solid was collected by filtration. Intermediate 2 (400 mg, 90%) was used for next step without further purification.

### Example 9

### tert-Butyl 4-(4-(5-(3-aminostyryl)pyrimidin-2-ylamino)phenylsulfonyl)piperidine-1-carboxylate (Intermediate 8)

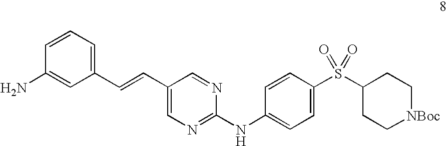

To a solution of intermediate 2 (200 mg, 0.82 mmol) in 1,4-dioxane (20 mL) was added intermediate 5 (334 mg, 0.82 mmol), Cs₂CO₃ (1.05 g, 3.2 mmol), Pd₂(dba)₃ (74 mg, 0.08 mmol), and 4,5-bis(diphenylphosphino)-9,9-dimethyxanthene (Xant Phos, 140 mg, 0.24 mmol). The mixture was heated under reflux overnight under Ar. The solid was filtered off and the filtrate washed with brine (1×50 mL). The organic solution was separated and dried (Na₂SO₄). The solvent was removed until 5 mL and hexanes (50 mL) added, the solid was collected by filtration. The solid was dissolved in EtOH (100 mL) and Na₂S.9H₂O (1.9 g, 8 mmol) was added. The reaction mixture was heated under reflux for 4 h. The solid was filtered off and solvent was removed in vacuo. The residue was suspended in EtOAc (100 mL) and brine (100 mL) was added. The organic layer was separated and the aqueous was extracted with EtOAc (2×50 mL). Combined organic layer was dried and solvent was removed in vacuo. The title intermediate was used for next step without further purification.

### Example 10

### N-(3-((E)-2-(2-(4-(Piperidin-4-ylsulfonyl)phenylamino)pyrimidin-5-yl)vinyl) (Compound I)

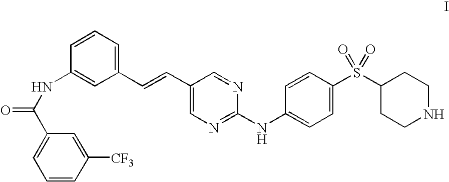

To a solution of intermediate 8 in anhydrous CH₂Cl₂ (10 mL) was added 3-(trifluoro-methyl)benzoyl chloride (205 mg, 0.98 mmol) and Et₃N (0.45 mL, 3.28 mmol). The mixture was stirred for 4 h at room temperature. The saturated NaHCO₃ (50 mL) was added. The organic layer was separated and the aqueous was extracted with CH₂Cl₂ (2×10 mL). The combined organic layer was dried (Na₂SO₄) and the solvent was removed in vacuo. The residue was dissolved in anhydrous CH₂Cl₂ (5 mL) and the trifluoroacetic acid (1 mL) was added. The reaction was stirred for 2 h at room temperature. The saturated NaHCO₃ (20 mL) was added. The organic layer was separated and aqueous was extracted with CH₂Cl₂ (2×10 mL). The combined organic layer was dried (Na₂SO₄) and the solvent was removed in vacuo. The residue was purified by HPLC to afford the title compound (16 mg of HCl salt, 3%) as a yellow solid.

¹H NMR (500 MHz, DMSO-d₆): δ 1.66-1.71 (m, 2H), 1.97-2.01 (m, 2H), 2.82-2.85 (m, 3H), 3.26-3.30 (m, 2H), 3.43-3.51 (m, 1H), 7.14 (br s, 1H), 7.35 (br s, 2H), 7.64 (br s, 1H), 7.72 (br s, 2H), 8.04 (br s, 2H), 8.30 (br s, 2H), 8.68 (br s, 1H), 8.86 (s, 2H), 9.29 (br s, 1H), 10.43 (s, 1H), 10.58 (s, 1H). MS (ES+): m/z 608 (M+H)⁺.

### Example 11

### 5-(3-Aminostyryl)-N-(4-(piperidin-4-ylsulfonyl)phenyl)pyrimidin-2-amine (Compound II)

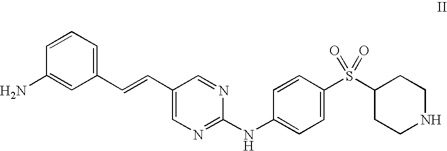

Intermediate 8 was dissolved in anhydrous CH₂Cl₂ (2 mL) and the BBr₃ (1 mL) was added. The reaction was stirred for 2 h at room temperature. The saturated NaHCO₃ (20 mL) was added. The organic layer was separated and aqueous was extracted with CH₂Cl₂ (2×10 mL). The combined organic layer was dried (Na₂SO₄) and the solvent was removed in vacuo. The residue was purified by HPLC to afford the title compound (10 mg of HCl salt, 6%) as a yellow solid.

¹H NMR (500 MHz, DMSO-d₆): δ 1.68-1.72 (m, 2H), 1.99-2.03 (m, 2H), 2.81-2.87 (m, 2H), 3.28-3.32 (m, 2H), 3.42-3.50 (m, 1H), 7.20 (d, J=16.6 Hz, 1H), 7.26 (d, J=7.0 Hz, 1H), 7.39 (d, J=16.6 Hz, 1H), 7.47-7.54 (m, 1H), 7.60 (d, J=7.0 Hz, 1H), 7.75 (d, J=8.7 Hz, 1H), 8.07 (d, J=8.7 Hz, 1H), 8.59 (d, J=4.6 Hz, 1H), 8.89 (s, 2H), 9.20 (br s, 1H), 10.49 (s, 1H). MS (ES+): m/z 436 (M+H)⁺.

### Example 12

### 5-(3,4-Dimethoxystyryl)pyrimidin-2-amine (Intermediate 9)

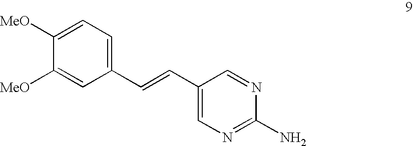

To a solution of 1,2-dimethoxy-4-vinylbenzene (164.2 mg, 1 mmol) in DMF (10 mL) was added 5-bromopyrimidin-2-amine (174 mg, 1 mmol), Pd(OAc)₂ (45 mg, 0.2 mmol), and Et₃N (0.55 mL, 4 mmol). The reaction was heated at 150° C. for 15 min in microwave. The solid was filtered off and washed with EtOAc. The filtrate was washed with brine (1×100 mL). The organic solution was separated. The aqueous phase was extracted with EtOAc (2×10 mL). The combined organic phase was dried (Na₂SO₄) and concentrated until 5 mL remaining. The hexanes (100 mL) were added to above solution and the solid was collected by filtration. The title intermediate was used for next step without further purification.

### Example 13

### 4-((E)-2-(2-(4-(Piperidin-4-ylsulfonyl)phenylamino)pyrimidin-5-yl)vinyl)benzene-1,2-diol (Compound III)

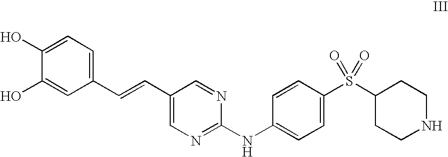

To a solution of intermediate 9 (1.0 mmol) in 1,4-dioxane (20 mL) was added intermediate 5 (404 mg, 1.0 mmol), Cs₂CO₃ (1.05 g, 3.2 mmol), Pd₂(dba)₃ (92 mg, 0.1 mmol), and 4,5-bis(diphenylphosphino)-9,9-dimethy-xanthene (Xant Phos, 173 mg, 0.3 mmol). The mixture was heated under reflux overnight under Ar. The solid was filtered off and the filtrate washed with brine (1×50 mL). The organic solution was separated and dried (Na₂SO₄). The solvent was removed until 5 mL and hexanes (50 mL) were added, the solid was collected by filtration. The solid was dissolved in anhydrous CH₂Cl₂ (2 mL) and the BBr₃ (1 mL) was added. The reaction was stirred for 2 h at room temperature. The saturated NaHCO₃ (20 mL) was added. The organic layer was separated and aqueous was extracted with CH₂Cl₂ (2×10 mL). The combined organic layer was dried (Na₂SO₄) and the solvent was removed in vacuo. The residue was purified by HPLC to afford the title compound (85 mg of HCl salt, 18%) as a yellow solid.

¹H NMR (500 MHz, DMSO-d₆): δ 1.72-1.77 (m, 2H), 1.99-2.01 (m, 2H), 2.81-2.87 (m, 2H), 3.28-3.30 (m, 2H), 3.45-3.52 (m, 1H), 6.76 (d, J=8.1 Hz, 1H), 6.83-6.86 (m, 2H), 7.00 (d, J=2.2 Hz, 1H), 7.16 (d, J=16.6 Hz, 1H), 7.73 (d, J=8.9 Hz, 2H), 8.06 (d, J=8.9 Hz, 1H), 8.80 (s, 2H), 8.87 (br s, 1H), 9.51 (br s, 1H), 10.39 (s, 1H). MS (ES+): m/z 453 (M+H)⁺.

### Example 14

### 2-(4-(3-Bromophenyl)piperazin-1-yl)ethanol (Intermediate 10)

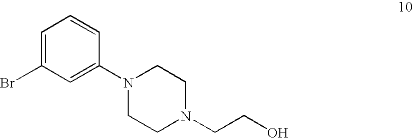

To a solution of 1-(3-bromophenyl)piperazine (1 g, 4.1 mmol) in DMF was added 2-bromoethanol (0.35 mL, 5 mmol) and K₂CO₃ (2.3 g, 17 mmol). The mixture was stirred overnight at room temperature. The solid was filtered off and washed with CH₂Cl₂. The filtrate was concentrated in vacuo. The residue was dissolved in CH₂Cl₂ (100 mL) and washed with brine (2×50 mL). The organic layer was collected and dried (Na₂SO₄). The solvent was removed in vacuo to afford crude product for next step.

### Example 15

### 5-((E)-2-(1H-Indol-4-yl)vinyl)pyrimidin-2-amine (Intermediate 11)

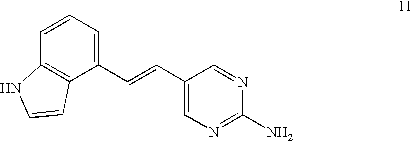

To a solution of intermediate 1 (1.83 g, 15.1 mmol) and 4-bromo-indole-1-carboxylic acid tert-butyl ester (4.92 g, 16.62 mmol) in DMF (100 mL) under argon atmosphere was added Pd(OAc)₂ (0.34 g, 1.51 mmol), PPh₃ (0.79 g, 3.02 mmol) and NaHCO₃ (2.54 g, 30.2 mmol). The reaction mixture was heated to 150° C. and stirred at 150° C. for 4 h. After cooling to room temperature, the reaction mixture was poured into 1:1 EtOAc/H₂O mixture (400 mL). The biphasic mixture was shaken and filtered though a short pad of silica gel. The organic layer was separated, washed with brine (4×200 mL), dried over anhydrous Na₂SO₄, filtered and concentrated in vacuo. The resulting residue was heated in ca. 50 mL of EtOAc, filtered, washed with Et₂O and dried in vacuo to afford the title intermediate as a tan solid (1.77 g, 50% yield).

¹H NMR (500 MHz, DMSO-d₆): δ 6.76 (br s, 2H), 6.88-6.89 (m, 1H), 7.09 (t, J=7.7 Hz, 1H), 7.13 (d, J=16.6 Hz, 1H), 7.26 (d, J=7.3 Hz, 1H), 7.30 (d, J=8.0 Hz, 1H), 7.40 (t, J=2.8 Hz, 1H), 7.48 (d, J=16.7 Hz, 1H), 8.60 (s, 2H), 11.17 (s, 1H). ¹³C NMR (125 MHz, DMSO-d₆): δ 100.1, 110.6, 116.1, 120.3, 121.1, 122.5, 124.4, 125.4, 125.9, 128.8, 136.3, 156.0, 162.6. MS (ES+): m/z 237 (M+H)⁺.

### Example 16

### 2-(4-(3-(5-((E)-2-(1H-Indol-4-yl)vinyl)pyrimidin-2-ylamino)phenyl)piperazin-1-yl)ethanol (Compound IV)

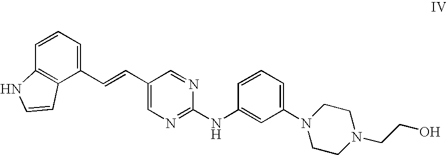

To a solution of intermediate 11 (124.2 mg, 0.52 mmol) in 1,4-dioxane (20 mL) was added intermediate 10 (180 mg, 0.63 mmol), Cs₂CO₃ (660 mg, 2.0 mmol), Pd₂(dba)₃ (46 mg, 0.05 mmol), and 4,5-bis(diphenylphosphino)-9,9-dimethy-xanthene (Xant Phos, 87 mg, 0.15 mmol). The mixture was heated under reflux overnight under Ar. The solid was filtered off and the filtrate washed with brine (1×50 mL). The organic solution was separated and dried (Na₂SO₄). The solvent was removed in vacuo. The residue was purified by HPLC to afford the title compound (30 mg of HCl salt, 12%) as a yellow solid.

¹H NMR (500 MHz, DMSO-d₆): δ 1.72-1.77 (m, 2H), 1.99-2.01 (m, 2H), 2.81-2.87 (m, 2H), 3.28-3.30 (m, 2H), 6.76 (d, J=8.1 Hz, 1H), 6.83-6.86 (m, 2H), 7.00 (d, J=2.2 Hz, 1H), 7.16 (d, J=16.6 Hz, 1H), 7.73 (d, J=8.9 Hz, 2H), 8.06 (d, J=8.9 Hz, 1H), 8.80 (s, 2H), 8.87 (br s, 1H), 9.51 (br s, 1H), 10.39 (s, 1H). MS (ES+): m/z 453 (M+H)⁺.

### Example 17

### 2-(4-(4-(5-((E)-2-(1H-Indol-4-yl)vinyl)pyrimidin-2-ylamino)phenylsulfonyl)piperidin-1-yl)ethanol (Compound V))

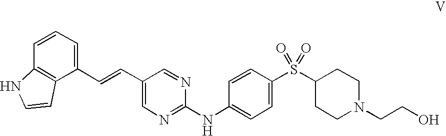

To a solution of compound LXXX described in detail below in the application (46 mg, 0.1 mmol) in DMF (5 mL) was added 2-bromoethanol (8.5 μL, 0.12 mmol) and N,N-diisoprypylethylamine (52 μL, 0.4 mmol). The mixture was stirred overnight at room temperature. The reaction mixture was concentrated and the residue was purified by HPLC to afford the title compound (22 mg of HCl salt, 41%) as a yellow solid.

¹H NMR (500 MHz, DMSO-d₆): δ 1.82-1.91 (m, 2H), 2.01-2.09 (m, 2H), 2.93-2.98 (m, 2H), 3.06-3.09 (m, 2H), 3.42-3.47 (m, 1H), 3.56-3.59 (m, 2H), 3.70 (t, J=4.9 Hz, 2H), 6.94 (s, 1H), 7.12 (t, J=7.7 Hz, 1H), 7.26 (d, J=16.7 Hz, 1H), 7.32 (d, J=7.3 Hz, 1H), 7.35 (d, J=8.1 Hz, 1H), 7.44 (t, J=2.8 Hz, 1H), 7.70 (d, J=16.7 Hz, 1H), 7.76 (d, J=9.0 Hz, 2H), 8.10 (d, J=9.0 Hz, 2H), 8.96 (s, 2H), 9.79 (br s, 1H), 10.46 (s, 1H), 11.26 (s, 1H). MS (ES+): m/z 504 (M+H)⁺.

### Example 18

### 5-((E)-2-(6-Aminopyridin-2-yl)vinyl)-N-(4-(piperidin-4-Ylsulfonyl)phenyl)pyrimidin-2-amine (Compound VI)

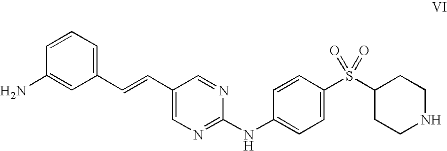

To a solution of intermediate 6 (78 mg, 0.17 mmol) in DMF (5 mL) was added 6-bromopyridin-2-amine (33 mg, 0.19 mmol), Pd(OAc)₂ (8 mg, 0.35 mmol), and Et₃N (95 μL, 0.68 mmol). The reaction was heated at 180° C. for 30 min in microwave. The solid was filtered off and washed with EtOAc. The filtrate was washed with brine (1×100 mL). The organic solution was separated. The aqueous was extracted with EtOAc (2×10 mL). The combined organic phase was dried (Na₂SO₄) and concentrated. The residue was dissolved in anhydrous CH₂Cl₂ (10 mL) and the TFA (2 mL) was added. The reaction was stirred for 4 h at room temperature. The 10% NaOH (20 mL) was added. The organic layer was separated and aqueous was extracted with CH₂Cl₂ (2×10 mL). The combined organic layer was dried (Na₂SO₄) and the solvent was removed in vacuo. The residue was purified by HPLC to afford the title compound (6 mg of HCl salt, 7%) as a yellow solid.

¹H NMR (500 MHz, DMSO-d₆): δ 1.66-1.74 (m, 2H), 2.00-2.02 (m, 2H), 2.82-2.87 (m, 2H), 3.31-3.33 (m, 2H), 3.42-3.47 (m, 1H), 6.88 (d, J=8.5 Hz, 1H), 6.96 (d, J=7.2 Hz, 1H), 7.28 (d, J=16.7 Hz, 1H), 7.77 (d, J=8.6 Hz, 2H), 7.87 (d, J=16.7 Hz, 1H), 7.90(t, J=7.9 Hz, 1H), 8.09 (d, J=8.6 Hz, 2H), 8.07 (br s, 1H), 8.54 (br s, 1H), 8.85 (s, 2H), 9.06 (br s, 1H), 10.67 (s, 1H). MS (ES+): m/z 437 (M+H)⁺.

### Example 19

### 3-(2-{2-[4-(Piperidine-4-sulfonyl)-phenylamino]-pyrimidin-5-yl}-vinyl)-benzene-sulfonamide (Compound VII)

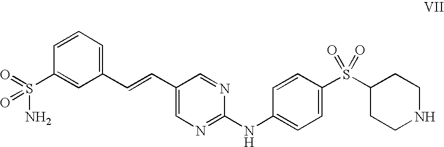

To a solution of intermediate 6 (63 mg, 0.14 mmol) in DMF (5 mL) was added 3-bromo-benzenesulfonamide (37 mg, 0.16 mmol), Pd(OAc)₂ (8 mg, 0.35 mmol), and Et₃N (95 μL, 0.68 mmol). The reaction was heated at 180° C. for 30 min in microwave. The solid was filtered off and washed with EtOAc. The filtrate was washed with brine (1×100 mL). The organic solution was separated. The aqueous was extracted with EtOAc (2×10 mL). The combined organic phase was dried (Na₂SO₄) and concentrated. The residue was dissolved in anhydrous CH₂Cl₂ (10 mL) and the TFA (2 mL) was added. The reaction was stirred for 4 h at room temperature. The 10% NaOH (20 mL) was added. The organic layer was separated and aqueous was extracted with CH₂Cl₂ (2×10 mL). The combined organic layer was dried (Na₂SO₄) and the solvent was removed in vacuo. The residue was purified by HPLC to afford the title compound (4 mg of HCl salt, 6%) as a yellow solid.

¹H NMR (500 MHz, DMSO-d₆): δ 1.66-1.73 (m, 2H), 2.01-2.03 (m, 2H), 2.81-2.89 (m, 2H), 3.31-3.34 (m, 2H), 3.42-3.47 (m, 1H), 7.29 (d, J=16.6 Hz, 1H), 7.41 (s, 2H), 7.46 (d, J=16.6 Hz, 1H), 7.60 (t, J=7.8 Hz, 1H), 7.73 (d, J=7.8 Hz, 1H), 7.76 (d, J=8.9 Hz, 2H), 7.79 (d, J=7.8 Hz, 1H), 8.02 (s, 1H), 8.08 (d, J=8.9 Hz, 2H), 8.46 (br s, 1H), 8.90 (s, 2H), 8.98 (br s, 1H), 10.49 (s, 1H). MS (ES+): m/z 500 (M+H)⁺.

### Example 20

### 4-(4-Bromo-benzenesulfonylamino)-piperidine-1-carboxylic acid tert-butyl ester (Intermediate 12)

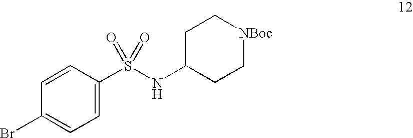

To a solution of 4-bromobenzenesulfonyl chloride (1.28 g, 5 mmol) in CH₂Cl₂ (100 mL) was added tert-butyl 4-aminopiperidine-1-carboxylate (1.2 g, 6 mmol) and Et₃N (2.8 mL, 20 mmol). The reaction mixture was stirred overnight at room temperature. The saturated NaHCO₃ (100 mL) was added. The organic layer was separated and aqueous was extracted with CH₂Cl₂ (2×30 mL). The combined organic layer was dried (Na₂SO₄). The solvent was removed in vacuo to afford the title product (2.0 g, 95%) as yellow solid.

### Example 21

### 5-(3-Methoxystyryl)pyrimidin-2-amine (Intermediate 13)

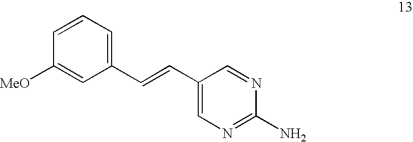

To a solution of intermediate 1 (62 mg, 0.5 mmol) in DMF (5 mL) was added 1-bromo-3-methoxybenzene (112 mg, 0.6 mmol), Pd(OAc)₂ (23 mg, 0.1 mmol), and Et₃N (281 μL, 2.0 mmol). The reaction was heated at 180° C. for 30 min in microwave. The solid was filtered off and washed with EtOAc. The filtrate was washed with brine (1×100 mL). The organic solution was separated. The aqueous was extracted with EtOAc (2×10 mL). The combined organic phase was dried (Na₂SO₄) and concentrated until 5 mL. The hexanes (100 mL) were added. The solid was collected to afford the title intermediate (98 mg, 86%) as black solid.

### Example 22

### 4-{5-[2-(3-Hydroxy-phenyl)-vinyl]-pyrimidine-2-ylamino}-N-piperdin-4-yl-benzene-sulfonamide (Compound VIII)

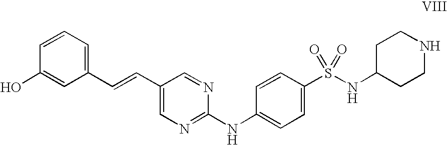

To a solution of intermediate 13 (227 mg, 1.0 mmol) in 1,4-dioxane (20 mL) was added intermediate 12 (419 mg, 1.0 mmol), Cs₂CO₃ (1.3 g, 4 mmol), Pd₂(dba)₃ (92 mg, 0.1 mmol), and 4,5-bis(diphenylphosphino)-9,9-dimethy-xanthene (Xant Phos, 173 mg, 0.3 mmol). The mixture was heated under reflux overnight under Ar. The solid was filtered off and the filtrate washed with brine (1×50 mL). The organic solution was separated and dried (Na₂SO₄). The solvent was removed until 5 mL and hexanes (50 mL) added, the solid was collected by filtration. The solid was dissolved in anhydrous CH₂Cl₂ (2 mL) and the BBr₃ (1 mL) was added. The reaction was stirred for 2 h at room temperature. The saturated NaHCO₃ (20 mL) was added. The organic layer was separated and aqueous was extracted with CH₂Cl₂ (2×10 mL). The combined organic layer was dried (Na₂SO₄) and the solvent was removed in vacuo. The residue was purified by HPLC to afford the title compound (32 mg of HCl salt, 7%) as a yellow solid.

¹H NMR (500 MHz, DMSO-d₆): δ 1.51-1.58 (m, 2H), 1.71-1.75 (m, 2H), 2.83-2.90 (m, 2H), 3.13-3.15 (m, 2H), 3.27 (br s, 1H), 6.70 (dd, J=2.2 Hz, J=8.1 Hz, 1H), 6.96 (s, 1H), 7.00 (d, J=7.9 Hz, 1H), 7.07 (d, J=16.6 Hz, 1H), 7.18 (t, J=7.8 Hz, 1H), 7.24 (d, J=16.6 Hz, 1H), 7.74 (d, J=8.9 Hz, 2H), 7.80 (d, J=6.9 Hz, 1H), 7.98 (d, J=8.9 Hz, 2H), 8.58 (br s, 1H), 8.75 (br s, 1H), 8.83 (s, 2H), 10.30 (s, 1H). MS (ES+): m/z 452 (M+H)⁺.

### Example 23

### 3-(4-Bromo-benzenesulfonylamino)-piperidine-1-carboxylic acid tert-butyl ester (Intermediate 14)

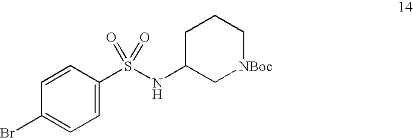

To a solution of 4-bromobenzenesulfonyl chloride (582 mg, 2.3 mmol) in CH₂Cl₂ (100 mL) was added tert-butyl 3-aminopiperidine-1-carboxylate (380 mg, 1.9 mmol) and Et₃N (1.2 mL, 8 mmol). The reaction mixture was stirred overnight at room temperature. The saturated NaHCO₃ (100 mL) was added. The organic layer was separated and aqueous was extracted with CH₂Cl₂ (2×30 mL). The combined organic layer was dried (Na₂SO₄). The solvent was removed in vacuo to afford the title intermediate (856 mg, 91%) as yellow solid.

### Example 24

### 4-{5-[2-(3-Hydroxy-phenyl)-vinyl]-pyrimidine-2-ylamino}-N-piperdin-4-yl-benzene-sulfonamide (Compound IX)

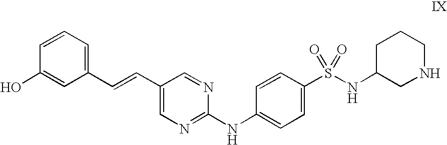

To a solution of intermediate 13 (159 mg, 0.7 mmol) in 1,4-dioxane (20 mL) was added intermediate 14 (293 mg, 0.7 mmol), Cs₂CO₃ (913 mg, 2.8 mmol), Pd₂(dba)₃ (64 mg, 0.07 mmol), and 4,5-bis(diphenylphosphino)-9,9-dimethy-xanthene (Xant Phos, 122 mg, 0.21 mmol). The mixture was heated under reflux overnight under Ar. The solid was filtered off and the filtrate washed with brine (1×50 mL). The organic solution was separated and dried (Na₂SO₄). The solvent was removed until 5 mL and hexanes (50 mL) added, the solid was collected by filtration. The solid was dissolved in anhydrous CH₂Cl₂ (2 mL) and the BBr₃ (1 mL) was added. The reaction was stirred for 2 h at room temperature. The saturated NaHCO₃ (20 mL) was added. The organic layer was separated and aqueous was extracted with CH₂Cl₂ (2×10 mL). The combined organic layer was dried (Na₂SO₄) and the solvent was removed in vacuo. The residue was purified by HPLC to afford the title compound (12 mg of HCl salt, 4%) as a yellow solid.

¹H NMR (500 MHz, DMSO-d₆): δ 1.34-1.41 (m, 1H), 1.50-1.55 (m, 1H), 1.62-1.67 (m, 1H), 1.72-1.76 (m, 1H), 2.62-2.67 (m, 1H), 2.70-2.75 (m, 1H), 3.04-3.06 (m, 2H), 3.26-3.33 (m, 1H), 6.71 (dd, J=2.3 Hz, J=8.5 Hz, 1H), 6.96 (t, J=2.0 Hz, 1H), 7.00 (d, J=7.8 Hz, 1H), 7.08 (d, J=16.6 Hz, 1H), 7.18 (t, J=7.8 Hz, 1H), 7.24 (d, J=16.6 Hz, 1H), 7.76 (d, J=8.9 Hz, 2H), 7.91 (d, J=6.6 Hz, 1H), 8.00 (d, J=8.9 Hz, 2H), 8.83 (s, 2H), 8.86 (br s, 1H), 8.93 (br s, 1H), 10.33 (s, 1H). MS (ES+): m/z 452 (M+H)⁺.

### Example 25

### N-(6-Bromobenzo[d]thiazol-2-yl)-3-(trifluoromethyl)benzamide (Intermediate 15)

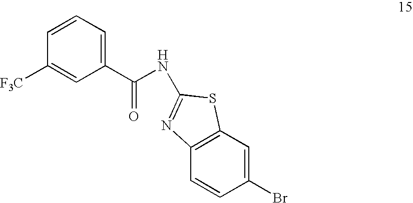

To a solution of 6-bromobenzo[d]thiazol-2-amine (115 mg, 0.5 mmol) in CH₂Cl₂ (20 mL) was added 3-(trifluoromethyl)benzoyl chloride (125 mg, 0.6 mmol), DMAP (12 mg, 0.1 mmol) and Et₃N (0.28 mL, 2.0 mmol). The reaction mixture was stirred overnight at room temperature. The saturated NaHCO₃ (50 mL) was added. The organic layer was separated and aqueous was extracted with CH₂Cl₂ (2×30 mL). The combined organic layer was dried (Na₂SO₄). The solvent was removed in vacuo. The residue was purified by flash column (SiO₂/50% EtOAc in hexanes) to afford the title intermediate (180 mg, 90%) as yellow solid.

### Example 26

### N-(6-((E)-2-(2-(4-(Piperidin-4-ylsulfonyl)phenylamino)pyrimidin-5-yl)vinyl)benzo[d]thiazol-2-yl)-3-(trifluoromethyl)benzamide (Compound X)

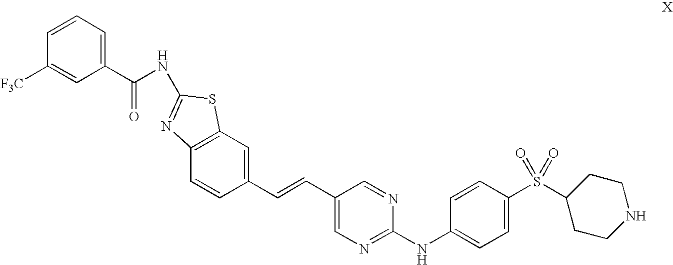

To a solution of intermediate 6 (88 mg, 0.22 mmol) in DMF (5 mL) was added intermediate 15 (80 mg, 0.22 mmol), Pd(OAc)₂ (8 mg, 0.35 mmol), and Et₃N (95 μL, 0.68 mmol). The reaction was heated at 180° C. for 30 min in microwave. The solid was filtered off and washed with EtOAc. The filtrate was washed with brine (1×100 mL). The organic solution was separated. The aqueous was extracted with EtOAc (2×10 mL). The combined organic phase was dried (Na₂SO₄) and concentrated. The residue was dissolved in anhydrous CH₂Cl₂ (10 mL) and the TFA (2 mL) was added. The reaction was stirred for 4 h at room temperature. The 10% NaOH (20 mL) was added. The organic layer was separated and aqueous was extracted with CH₂Cl₂ (2×10 mL). The combined organic layer was dried (Na₂SO₄) and the solvent was removed in vacuo. The residue was purified by HPLC to afford the title compound (4 mg of HCl salt, 3%) as a yellow solid.

¹H NMR (500 MHz, DMSO-d₆): δ 1.66-1.75 (m, 2H), 2.01-2.03 (m, 2H), 2.82-2.89 (m, 2H), 3.31-3.34 (m, 2H), 3.42-3.47 (m, 1H), 7.25 (d, J=16.6 Hz, 1H), 7.47 (d, J=16.6 Hz, 1H), 7.73 (d, J=8.7 Hz, 1H), 7.76 (d, J=8.9 Hz, 2H), 7.80-7.85 (m, 2H), 8.04 (d, J=7.6 Hz, 2H), 8.09 (d, J=8.9 Hz, 2H), 8.23 (s, 1H), 8.42 (d, J=8.1 Hz, 1H), 8.53 (s, 1H), 8.55 (br s, 1H), 8.88 (s, 2H), 9.12 (br s, 1H), 10.47 (s, 1H). MS (ES+): m/z 665 (M+H)⁺.

### Example 27

### N-(6-Bromobenzo[d]thiazol-2-yl)-3-fluorobenzamide (Intermediate 16)

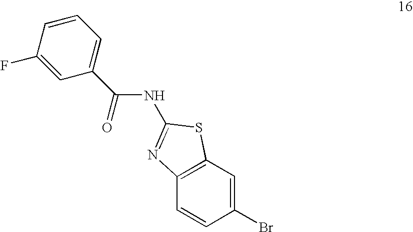

To a solution of 6-bromobenzo[d]thiazol-2-amine (229 mg, 1.0 mmol) in CH₂Cl₂ (20 mL) was added 3-fluoro-benzoyl chloride (190 mg, 1.2 mmol), DMAP (25 mg, 0.2 mmol) and Et₃N (0.56 mL, 4.0 mmol). The reaction mixture was stirred overnight at room temperature. The saturated NaHCO₃ (50 mL) was added. The organic layer was separated and aqueous was extracted with CH₂Cl₂ (2×30 mL). The combined organic layer was dried (Na₂SO₄). The solvent was removed in vacuo. The residue was purified by flash column (SiO₂/50% EtOAc in hexanes) to afford the title intermediate (340 mg, 97%) as yellow solid.

### Example 28

### N-(6-((E)-2-(2-(4-(Piperidin-4-ylsulfonyl)phenylamino)pyrimidin-5-yl)vinyl)benzo[d]thiazol-2-yl)-3-fluorobenzamide (Compound XI)

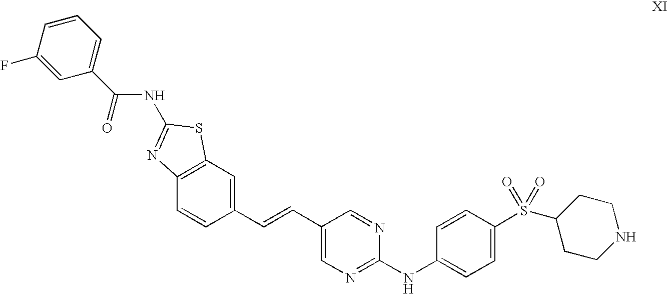

To a solution of intermediate 6 (253 mg, 0.57 mmol) in DMF (5 mL) was added intermediate 16 (240 mg, 0.68 mmol), Pd(OAc)₂ (23 mg, 0.1 mmol), and Et₃N (0.28 mL, 2.0 mmol). The reaction was heated at 180° C. for 40 min in microwave. The solid was filtered off and washed with EtOAc. The filtrate was washed with brine (1×100 mL). The organic solution was separated. The aqueous was extracted with EtOAc (2×10 mL). The combined organic phase was dried (Na₂SO₄) and concentrated. The residue was dissolved in anhydrous CH₂Cl₂ (10 mL) and the TFA (2 mL) was added. The reaction was stirred for 4 h at room temperature. The 10% NaOH (20 mL) was added. The organic layer was separated and aqueous was extracted with CH₂Cl₂ (2×10 mL). The combined organic layer was dried (Na₂SO₄) and the solvent was removed in vacuo. The residue was purified by HPLC to afford the title compound (6 mg of HCl salt, 2%) as a yellow solid.

¹H NMR (500 MHz, DMSO-d₆): δ 1.66-1.75 (m, 2H), 2.00-2.03 (m, 2H), 2.82-2.89 (m, 2H), 3.31-3.34 (m, 2H), 3.42-3.47 (m, 1H), 7.26 (d, J=16.6 Hz, 1H), 7.47 (d, J=16.6 Hz, 1H), 7.52-7.56 (m, 1H), 7.62-7.66 (m, 1H), 7.72 (d, J=8.4 Hz, 1H), 7.76 (d, J=8.9 Hz, 2H), 7.80 (d, J=8.4 Hz, 1H), 7.97 (d, J=6.6 Hz, 1H), 8.00 (d, J=8.1 Hz, 1H), 8.09 (d, J=8.9 Hz, 2H), 8.67 (br s, 1H), 8.88 (s, 2H), 9.24 (br s, 1H), 10.47 (s, 1H). MS (ES+): m/z 615 (M+H)⁺.

### Example 29

### N-(4-Bromophenyl)-3-(trifluoromethyl)benzamide (Intermediate 17)

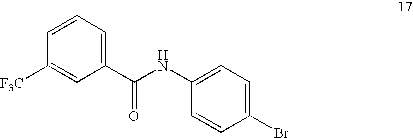

To a solution of 4-bromobenzenamine (1.24 g, 7.2 mmol) in CH₂Cl₂ (50 mL) was added 3-(trifluoromethyl)-benzoyl chloride (1.65 g, 7.9 mmol), DMAP (171 mg, 1.4 mmol) and Et₃N (4 mL, 28 mmol). The reaction mixture was stirred overnight at room temperature. The saturated NaHCO₃ (50 mL) was added. The organic layer was separated and aqueous was extracted with CH₂Cl₂ (2×30 mL). The combined organic layer was dried (Na₂SO₄). The solvent was removed in vacuo. The residue was purified by flash column (SiO₂/50% EtOAc in hexanes) to afford the title intermediate (2 g, 81%) as yellow solid.

### Example 30

### N-(4-((E)-2-(2-(4-(Piperidin-4-ylsulfonyl)phenylamino)pyrimidin-5-yl)vinyl)phenyl)-3-(trifluoromethyl)benzamide (Compound XII)

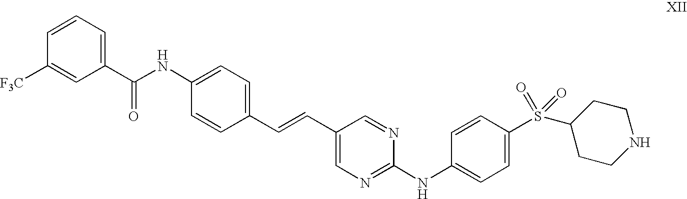

To a solution of intermediate 6 (430 mg, 0.97 mmol) in DMF (5 mL) was added 17 (440 mg, 1.2 mmol), Pd(OAc)₂ (45 mg, 0.2 mmol), and Et₃N (0.56 mL, 4.0 mmol). The reaction was heated at 180° C. for 40 min in microwave. The solid was filtered off and washed with EtOAc. The filtrate was washed with brine (1×100 mL). The organic solution was separated. The aqueous was extracted with EtOAc (2×10 mL). The combined organic phase was dried (Na₂SO₄) and concentrated. The residue was dissolved in anhydrous CH₂Cl₂ (10 mL) and the TFA (2 mL) was added. The reaction was stirred for 4 h at room temperature. The 10% NaOH (20 mL) was added. The organic layer was separated and aqueous was extracted with CH₂Cl₂ (2×10 mL). The combined organic layer was dried (Na₂SO₄) and the solvent was removed in vacuo. The residue was purified by HPLC to afford the title compound (44 mg of HCl salt, 7%) as a yellow solid.

¹H NMR (500 MHz, DMSO-d₆): δ 1.66-1.75 (m, 2H), 2.00-2.03 (m, 2H), 2.82-2.89 (m, 2H), 3.31-3.34 (m, 2H), 3.42-3.47 (m, 1H), 7.14 (d, J=16.6 Hz, 1H), 7.34 (d, J=16.6 Hz, 1H), 7.60 (d, J=8.7 Hz, 2H), 7.75 (d, J=8.9 Hz, 2H), 7.80 (t, J=7.9 Hz, 1H), 7.84 (d, J=8.7 Hz, 2H), 7.98 (d, J=8.0 Hz, 1H), 8.08 (d, J=8.9 Hz, 2H), 8.29 (d, J=8.2 Hz, 1H), 8.31 (s, 1H), 8.74 (br s, 1H), 8.85 (s, 2H), 9.14 (br s, 1H), 10.45 (s, 1H), 10.61 (s, 1H). MS (ES+): m/z 608 (M+H)⁺.

### Example 31

### 5-(3-(1H-Pyrazol-4-yl)styryl)-N-(4-(piperidin-4-ylsulfonyl)phenyl)pyrimidin-2-amine (Compound XIII)

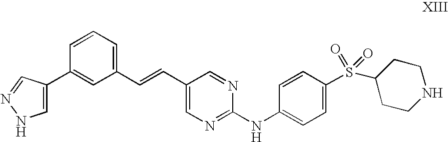

To a solution of intermediate 6 (223 mg, 0.5 mmol) in DMF (5 mL) was added 4-(3-bromophenyl)-1H-pyrazole (133 mg, 0.6 mmol), Pd(OAc)₂ (23 mg, 0.1 mmol), and Et₃N (0.28 mL, 2.0 mmol). The reaction was heated at 180° C. for 30 min in microwave. The solid was filtered off and washed with EtOAc. The filtrate was washed with brine (1×100 mL). The organic solution was separated. The aqueous was extracted with EtOAc (2×10 mL). The combined organic phase was dried (Na₂SO₄) and concentrated. The residue was dissolved in anhydrous CH₂Cl₂ (10 mL) and the TFA (2 mL) was added. The reaction was stirred for 4 h at room temperature. The 10% NaOH (20 mL) was added. The organic layer was separated and aqueous was extracted with CH₂Cl₂ (2×10 mL). The combined organic layer was dried (Na₂SO₄) and the solvent was removed in vacuo. The residue was purified by HPLC to afford the title compound (106 mg of HCl salt, 40%) as a yellow solid.

¹H NMR (500 MHz, DMSO-d₆): δ 1.70-1.78 (m, 2H), 1.98-2.01 (m, 2H), 2.80-2.87 (m, 2H), 3.28-3.30 (m, 2H), 3.48-3.52 (m, 1H), 7.27-7.38 (m, 4H), 7.52-7.54 (m, 1H), 7.75 (d, J=8.9 Hz, 2H), 7.86 (s, 1H), 8.09 (d, J=8.9 Hz, 2H), 8.13 (s, 2H), 8.86 (s, 2H), 8.95 (br s, 1H), 9.59 (br s, 1H), 10.48 (s, 1H). MS (ES+): m/z 487 (M+H)⁺.

### Example 32

### 5-((E)-2-(7-Fluoro-1H-indol-4-yl)vinyl)-N-(4-(piperidin-4-ylsulfonyl)phenyl)pyrimidin-2-amine (Compound XIV)

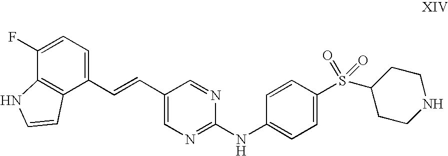

To a solution of intermediate 6 (445 mg, 1.0 mmol) in DMF (5 mL) was added 4-bromo-7-fluoro-1H-indole (214 mg, 1.0 mmol), Pd(OAc)₂ (45 mg, 0.2 mmol), and Et₃N (0.56 mL, 4.0 mmol). The reaction was heated at 180° C. for 60 min in microwave. The solid was filtered off and washed with EtOAc. The filtrate was washed with brine (1×100 mL). The organic solution was separated. The aqueous was extracted with EtOAc (2×10 mL). The combined organic phase was dried (Na₂SO₄) and concentrated. The residue was dissolved in anhydrous CH₂Cl₂ (10 mL) and the TFA (2 mL) was added. The reaction was stirred for 4 h at room temperature. The 10% NaOH (20 mL) was added. The organic layer was separated and aqueous was extracted with CH₂Cl₂ (2×10 mL). The combined organic layer was dried (Na₂SO₄) and the solvent was removed in vacuo. The residue was purified by HPLC to afford the title compound (23 mg of HCl salt, 4%) as a yellow solid.

¹H NMR (500 MHz, DMSO-d₆): δ 1.66-1.75 (m, 2H), 2.01-2.03 (m, 2H), 2.82-2.89 (m, 2H), 3.31-3.33 (m, 2H), 3.46-3.51 (m, 1H), 6.95 (d, J=8.3 Hz, 1H), 6.98 (d, J=8.3 Hz, 1H), 7.04 (dd, J=3.2 Hz, J=5.3 Hz, 1H), 7.22 (d, J=16.7 Hz, 1H), 7.51 (t, J=2.8 Hz, 1H), 7.66 (d, J=16.7 Hz, 1H), 7.75 (d, J=9.0 Hz, 2H), 8.09 (d, J=9.0 Hz, 2H), 8.61 (br s, 1H), 8.95 (s, 2H), 9.19 (br s, 1H), 10.44 (s, 1H), 11.75 (s, 1H). MS (ES+): m/z 478 (M+H)⁺.

### Example 33

### 5-((E)-2-(7-Chloro-1H-indol-4-yl)vinyl)-N-(4-(piperidin-4-ylsulfonyl)phenyl)pyrimidin-2-amine (Compound XV)

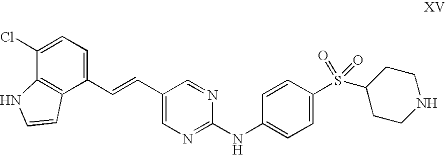

To a solution of intermediate 6 (223 mg, 0.5 mmol) in DMF (5 mL) was added 4-bromo-7-chloro-1H-indole (115 mg, 0.5 mmol), Pd(OAc)₂ (23 mg, 0.1 mmol), and Et₃N (0.28 mL, 2.0 mmol). The reaction was heated at 180° C. for 60 min in microwave. The solid was filtered off and washed with EtOAc. The filtrate was washed with brine (1×100 mL). The organic solution was separated. The aqueous was extracted with EtOAc (2×10 mL). The combined organic phase was dried (Na₂SO₄) and concentrated. The residue was dissolved in anhydrous CH₂Cl₂ (10 mL) and the TFA (2 mL) was added. The reaction was stirred for 4 h at room temperature. The 10% NaOH (20 mL) was added. The organic layer was separated and aqueous was extracted with CH₂Cl₂ (2×10 mL). The combined organic layer was dried (Na₂SO₄) and the solvent was removed in vacuo. The residue was purified by HPLC to afford the title compound (7 mg of HCl salt, 3%) as an orange solid.

¹H NMR (500 MHz, DMSO-d₆): δ 1.66-1.75 (m, 2H), 2.01-2.03 (m, 2H), 2.82-2.89 (m, 2H), 3.31-3.34 (m, 2H), 3.46-3.51 (m, 1H), 7.06 (dd, J=2.0 Hz, J=3.1 Hz, 1H), 7.20 (d, J=8.0 Hz, 1H), 7.29 (d, J=16.6 Hz, 1H), 7.34 (d, J=8.0 Hz, 1H), 7.51 (t, J=2.9 Hz, 1H), 7.66 (d, J=16.6 Hz, 1H), 7.76 (d, J=8.9 Hz, 2H), 8.09 (d, J=9.0 Hz, 2H), 8.56 (br s, 1H), 8.97 (s, 2H), 9.13 (br s, 1H), 10.46 (s, 1H), 11.58 (s, 1H). MS (ES+): m/z 494 (M+H)⁺.

### Example 34

### 5-((E)-2-(7-Methyl-1H-indol-4-yl)vinyl)-N-(4-(piperidin-4-ylsulfonyl)phenyl)pyrimidin-2-amine (Compound XVI)

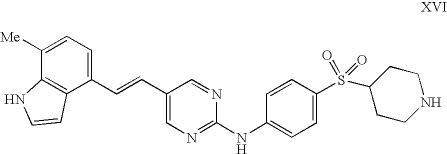

To a solution of intermediate 6 (223 mg, 0.5 mmol) in DMF (5 mL) was added 4-bromo-7-methyl-1H-indole (105 mg, 0.5 mmol), Pd(OAc)₂ (23 mg, 0.1 mmol), and Et₃N (0.28 mL, 2.0 mmol). The reaction was heated at 180° C. for 60 min in microwave. The solid was filtered off and washed with EtOAc. The filtrate was washed with brine (1×100 mL). The organic solution was separated. The aqueous was extracted with EtOAc (2×10 mL). The combined organic phase was dried (Na₂SO₄) and concentrated. The residue was dissolved in anhydrous CH₂Cl₂ (10 mL) and the TFA (2 mL) was added. The reaction was stirred for 4 h at room temperature. The 10% NaOH (20 mL) was added. The organic layer was separated and aqueous was extracted with CH₂Cl₂ (2×10 mL). The combined organic layer was dried (Na₂SO₄) and the solvent was removed in vacuo. The residue was purified by HPLC to afford the title compound (4 mg of HCl salt, 2%) as a yellow solid.

¹H NMR (500 MHz, DMSO-d₆): δ 1.64-1.75 (m, 2H), 2.01-2.04 (m, 2H), 2.48 (s, 3H), 2.82-2.89 (m, 2H), 3.31-3.34 (m, 2H), 3.46-3.51 (m, 1H), 6.92-6.95 (m, 2H), 7.20 (d, J=16.7 Hz, 1H), 7.23 (d, J=7.5 Hz, 1H), 7.43 (t, J=2.9 Hz, 1H), 7.65 (d, J=16.7 Hz, 1H), 7.75 (d, J=8.9 Hz, 2H), 8.09 (d, J=8.9 Hz, 2H), 8.43 (br s, 1H), 8.94 (s, 2H), 10.41 (s, 1H), 11.21 (s, 1H). MS (ES+): m/z 474 (M+H)⁺.

### Example 35

### 4-((E)-2-(2-(4-(Piperidin-4-ylsulfonyl)phenylamino)pyrimidin-5-yl)vinyl)benzo[d]thiazol-2-amine (Compound XVII)

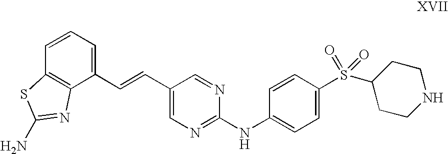

To a solution of intermediate 6 (240 mg, 0.54 mmol) in DMF (5 mL) was added 4-bromobenzo[d]thiazol-2-amine (124 mg, 0.54 mmol), Pd(OAc)₂ (23 mg, 0.1 mmol), and Et₃N (0.28 mL, 2.0 mmol). The reaction was heated at 180° C. for 60 min in microwave. The solid was filtered off and washed with EtOAc. The filtrate was washed with brine (1×100 mL). The organic solution was separated. The aqueous phase was extracted with EtOAc (2×10 mL). The combined organic phase was dried (Na₂SO₄) and concentrated. The residue was dissolved in anhydrous CH₂Cl₂ (10 mL) and the TFA (2 mL) was added. The reaction was stirred for 4 h at room temperature. The 10% NaOH (20 mL) was added. The organic layer was separated and aqueous was extracted with CH₂Cl₂ (2×10 mL). The combined organic layer was dried (Na₂SO₄) and the solvent was removed in vacuo. The residue was purified by HPLC to afford the title compound (9 mg of HCl salt, 3%) as a yellow solid.

¹H NMR (500 MHz, DMSO-d₆): δ 1.64-1.74 (m, 2H), 2.00-2.03 (m, 2H), 2.82-2.89 (m, 2H), 3.31-3.34 (m, 2H), 3.46-3.52 (m, 1H), 7.19 (t, J=7.8 Hz, 1H), 7.45 (d, J=16.5 Hz, 1H), 7.65 (d, J=7.8 Hz, 1H), 7.70 (d, J=7.2 Hz, 1H), 7.76 (d, J=9.1 Hz, 2H), 7.79 (d, J=16.5 Hz, 1H), 8.08 (d, J=9.1 Hz, 2H), 8.54 (br s, 1H), 8.88 (s, 2H), 9.11 (br s, 1H), 10.48 (s, 1H). MS (ES+): m/z 493 (M+H)⁺.

### Example 36

### 5-((E)-2-(1H-Indazol-3-yl)vinyl)-N-(4-(piperidin-4-ylsulfonyl)phenyl)pyrimidin-2-amine (Compound XVIII)

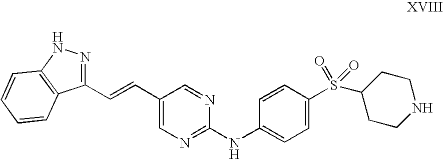

To a solution of intermediate 6 (445 mg, 1.0 mmol) in DMF (5 mL) was added 3-bromoindazole (197 mg, 1.0 mmol), Pd(OAc)₂ (45 mg, 0.2 mmol), and Et₃N (0.56 mL, 4.0 mmol). The reaction was heated at 180° C. for 60 min in microwave. The solid was filtered off and washed with EtOAc. The filtrate was washed with brine (1×100 mL). The organic solution was separated. The aqueous was extracted with EtOAc (2×10 mL). The combined organic phase was dried (Na₂SO₄) and concentrated. The residue was dissolved in anhydrous CH₂Cl₂ (10 mL) and the TFA (2 mL) was added. The reaction was stirred for 4 h at room temperature. The 10% NaOH (20 mL) was added. The organic layer was separated and aqueous phase was extracted with CH₂Cl₂ (2×10 mL). The combined organic layer was dried (Na₂SO₄) and the solvent was removed in vacuo. The residue was purified by HPLC to afford the title compound (45 mg of HCl salt, 9%) as a yellow solid.

¹H NMR (500 MHz, DMSO-d₆): δ 1.66-1.75 (m, 2H), 2.01-2.03 (m, 2H), 2.82-2.89 (m, 2H), 3.31-3.34 (m, 2H), 3.46-3.52 (m, 1H), 7.22 (t, J=7.5 Hz, 1H), 7.41 (t, J=7.2 Hz, 1H), 7.45 (d, J=16.9 Hz, 1H), 7.56 (d, J=8.4 Hz, 1H), 7.66 (d, J=16.9 Hz, 1H), 7.76 (d, J=9.0 Hz, 2H), 8.09 (d, J=9.0 Hz, 2H), 8.19 (d, J=8.2 Hz, 1H), 8.55 (br s, 1H), 8.98 (s, 2H), 9.13 (br s, 1H), 10.46 (s, 1H). MS (ES+): m/z 461 (M+H)⁺.

### Example 37

### 3-[4-(5-Vinyl-pyrimidin-2-ylamino)-benzenesulfonylamino]-piperidine-1-carboxylic acid tert-butyl ester (Intermediate 18)

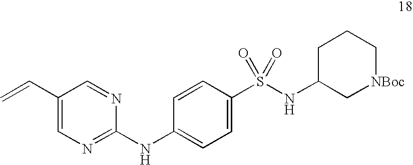

To a solution of intermediate 1 (166 mg, 1.36 mmol) in 1,4-dioxane (100 mL) was added intermediate 14 (570 mg, 1.36 mmol), Cs₂CO₃ (1.5 g, 4.6 mmol), Pd₂(dba)₃ (119 mg, 0.13 mmol), and 4,5-bis(diphenylphosphino)-9,9-dimethy-xanthene (Xant Phos, 226 mg, 0.39 mmol). The mixture was heated under reflux overnight under Ar. The solid was filtered off and the filtrate washed with brine (1×150 mL). The organic solution was separated and dried (Na₂SO₄). The solvent was removed until 5 mL and hexanes (100 mL) added. The solid (600 mg, 96%) was collected by filtration.

### Example 38

### 4-{5-[2-(2-Aminobenzothiazol-6-yl)-vinyl]-pyrimidin-2-ylamino}-N-piperidin-3-yl-benzenesulfonamide (Compound XIX)

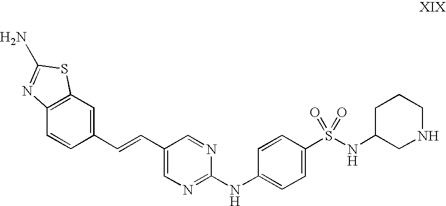

To a solution of intermediate 18 (120 mg, 0.26 mmol) in DMF (5 mL) was added 6-bromobenzo[d]thiazol-2-amine (60 mg, 0.26 mmol), Pd(OAc)₂ (12 mg, 0.05 mmol), and Et₃N (0.14 mL, 1.0 mmol). The reaction was heated at 180° C. for 60 min in microwave. The solid was filtered off and washed with EtOAc. The filtrate was washed with brine (1×100 mL). The organic solution was separated. The aqueous was extracted with EtOAc (2×10 mL). The combined organic phase was dried (Na₂SO₄) and concentrated. The residue was dissolved in anhydrous CH₂Cl₂ (10 mL) and the TFA (2 mL) was added. The reaction was stirred for 4 h at room temperature. The 10% NaOH (20 mL) was added. The organic layer was separated and aqueous was extracted with CH₂Cl₂ (2×10 mL). The combined organic layer was dried (Na₂SO₄) and the solvent was removed in vacuo. The residue was purified by HPLC to afford the title compound (4 mg of HCl salt, 3%) as a yellow solid.

¹H NMR (500 MHz, DMSO-d₆): δ 1.34-1.41 (m, 1H), 1.50-1.55 (m, 1H), 1.62-1.67 (m, 1H), 1.72-1.76 (m, 1H), 2.62-2.67 (m, 1H), 2.70-2.75 (m, 1H), 3.04-3.06 (m, 2H), 3.26-3.33 (m, 1H), 7.11 (d, J=16.6 Hz, 1H), 7.34 (d, J=16.6 Hz, 1H), 7.42 (d, J=8.4 Hz, 1H), 7.55 (d, J=8.4 Hz, 1H), 7.75 (d, J=8.9 Hz, 2H), 7.98 (s, 1H), 8.00 (d, J=8.9 Hz, 2H), 8.72 (br s, 1H), 8.80 (br s, 1H), 8.82 (s, 2H), 10.33 (s, 1H). MS (ES+): m/z 508 (M+H)⁺.

### Example 39

### tert-Butyl 4-(methylamino)piperidine-1-carboxylate (Intermediate 19)

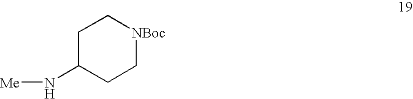

To a solution of tert-butyl 4-aminopiperidine-1-carboxylate (200.3 mg, 1.0 mmol) in CH₂Cl₂ (50 mL) was added MeI (213 mg, 1.5 mmol), and Et₃N (0.56 mL, 4 mmol). The reaction mixture was stirred overnight at room temperature. The saturated NaHCO₃ (50 mL) was added. The organic layer was separated and aqueous was extracted with CH₂Cl₂ (2×30 mL). The combined organic layer was dried (Na₂SO₄). The solvent was removed in vacuo. The residue was used for next step without purification.

### Example 40

### 4-[(4-Bromo-benzensulfonyl)-methyl-amino]-piperdine-1-carboxylic acid tert-butyl ester (Intermediate 20)

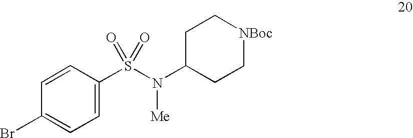

To a solution of intermediate 19 in CH₂Cl₂ (50 mL) was added 4-bromobenzenesulfonyl chloride (255 mg, 1.0 mmol), and Et₃N (0.56 mL, 4.0 mmol). The reaction mixture was stirred overnight at room temperature. The saturated NaHCO₃ (100 mL) was added. The organic layer was separated and aqueous was extracted with CH₂Cl₂ (2×30 mL). The combined organic layer was dried (Na₂SO₄). The solvent was removed in vacuo to afford crude product as yellow solid.

### Example 41

### 4-{Methyl-[4-(5-vinyl-pyrimidin-2-ylamino)-benzenesulfonyl]-amino}-piperidine-1-carboxylic acid tert-butyl ester (Intermediate 21)

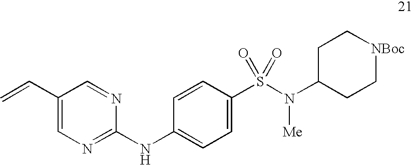

To a solution of intermediate 1 (121 mg, 1.0 mmol) in 1,4-dioxane (100 mL) was added intermediate 20 (1.0 mmol), Cs₂CO₃ (1.3 g, 4.0 mmol), Pd₂(dba)₃ (92 mg, 0.1 mmol), and 4,5-bis(diphenylphosphino)-9,9-dimethy-xanthene (Xant Phos, 180 mg, 0.3 mmol). The mixture was heated under reflux overnight under Ar. The solid was filtered off and the filtrate washed with brine (1×150 mL). The organic solution was separated and dried (Na₂SO₄). The solvent was removed and the residue was purified by flash column (SiO₂/50% EtOAc in hexanes) to afford the title intermediate (140 mg, 30%) as yellow oil.

### Example 42

### 4-{5-[2-(2-Amino-benzothiazol-6-yl)-vinyl]pyrimidin-2-ylamino}-N-methyl-N-piperidin-4-yl-benzensulfonamide (Compound XX)

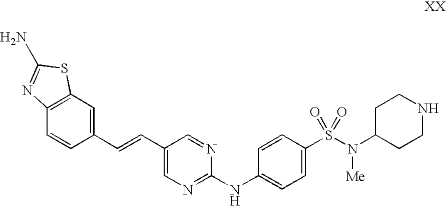

To a solution of intermediate 21 (140 mg, 0.3 mmol) in DMF (5 mL) was added 6-bromobenzo[d]thiazol-2-amine (68 mg, 0.3 mmol), Pd(OAc)₂ (14 mg, 0.06 mmol), and Et₃N (0.14 mL, 1.0 mmol). The reaction was heated at 180° C. for 60 min in microwave. The solid was filtered off and washed with EtOAc. The filtrate was washed with brine (1×50 mL). The organic solution was separated. The aqueous was extracted with EtOAc (2×10 mL). The combined organic phase was dried (Na₂SO₄) and concentrated. The residue was dissolved in anhydrous CH₂Cl₂ (10 mL) and the TFA (2 mL) was added. The reaction was stirred for 4 h at room temperature. The 10% NaOH (20 mL) was added. The organic layer was separated and aqueous was extracted with CH₂Cl₂ (2×10 mL). The combined organic layer was dried (Na₂SO₄) and the solvent was removed in vacuo. The residue was purified by HPLC to afford the title compound (10 mg of HCl salt, 6%) as a yellow solid.

¹H NMR (500 MHz, DMSO-d₆): δ 1.42-1.44 (m, 2H), 1.79-1.86 (m, 2H), 2.66 (s, 3H), 2.92-2.99 (m, 2H), 3.22-3.25 (m, 2H), 3.26-3.33 (m, 1H), 7.12 (d, J=16.6 Hz, 1H), 7.36 (d, J=16.6 Hz, 1H), 7.45 (d, J=8.4 Hz, 1H), 7.57 (d, J=8.4 Hz, 1H), 7.75 (d, J=8.9 Hz, 2H), 8.01 (s, 1H), 8.02 (d, J=8.9 Hz, 2H), 8.57 (br s, 1H), 8.80 (br s, 1H), 8.83 (s, 2H), 10.35 (s, 1H). MS (ES+): m/z 522 (M+H)⁺.

### Example 43

### 5-(3-Methoxystyryl)pyridin-2-amine (Intermediate 22)

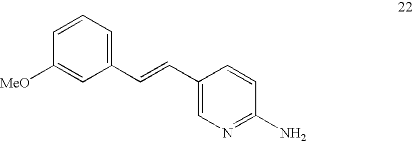

To a solution of 5-bromopyridin-2-amine (346 mg, 2.0 mmol) in DMF (10 mL) was added 1-methoxy-3-vinylbenzene (322 mg, 2.4 mmol), Pd(OAc)₂ (90 mg, 0.4 mmol), and Et₃N (1.12 mL, 8.0 mmol). The reaction was heated at 180° C. for 60 min in microwave. The solid was filtered off and washed with EtOAc. The filtrate was washed with brine (1×50 mL). The organic solution was separated. The aqueous was extracted with EtOAc (2×10 mL). The combined organic phase was dried (Na₂SO₄) and concentrated until 5 mL. The hexanes (100 mL) were added. The solid was collected to afford the title intermediate (300 mg, 66%) as black solid.

### Example 44

### 3-((E)-2-(6-(4-(Piperidin-4-ylsulfonyl)phenylamino)pyridin-3-yl)vinyl)phenol (Compound XXI)

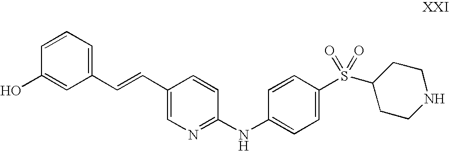

To a solution of intermediate 22 (228 mg, 1.0 mmol) in 1,4-dioxane (20 mL) was added 5 (404 mg, 1.0 mmol), Cs₂CO₃ (1.3 g, 4 mmol), Pd₂(dba)₃ (92 mg, 0.1 mmol), and 4,5-bis(diphenylphosphino)-9,9-dimethy-xanthene (Xant Phos, 173 mg, 0.3 mmol). The mixture was heated under reflux overnight under Ar. The solid was filtered off and the filtrate washed with brine (1×50 mL). The organic solution was separated and dried (Na₂SO₄). The solvent was removed until 5 mL and hexanes (50 mL) added, the solid was collected by filtration. The solid was dissolved in anhydrous CH₂Cl₂ (2 mL) and the BBr₃ (1 mL) was added. The reaction was stirred for 2 h at room temperature. The saturated NaHCO₃ (20 mL) was added. The organic layer was separated and aqueous was extracted with CH₂Cl₂ (2×10 mL). The combined organic layer was dried (Na₂SO₄) and the solvent was removed in vacuo. The residue was purified by HPLC to afford the title compound (14 mg of HCl salt, 3%) as a yellow solid.

¹H NMR (500 MHz, DMSO-d₆): δ 1.66-1.75 (m, 2H), 2.00-2.03 (m, 2H), 2.82-2.89 (m, 2H), 3.30-3.33 (m, 2H), 3.44-3.49 (m, 1H), 6.68 (dd, J=2.2 Hz, J=8.3 Hz, 1H), 6.96 (t, J=2.0 Hz, 1H), 7.00 (d, J=7.8 Hz, 1H), 7.06 (d, J=10.0 Hz, 1H), 7.11 (d, J=2.7 Hz, 1H), 7.16 (t, J=7.8 Hz, 1H), 7.71 (d, J=9.0 Hz, 2H), 8.00 (d, J=9.0 Hz, 2H), 8.02 (dd, J=2.4 Hz, J=8.9 Hz, 1H), 8.39 (d, J=2.3 Hz, 1H), 8.59 (br s, 1H), 9.18 (br s, 1H), 10.11 (s, 1H). MS (ES+): m/z 436 (M+H)⁺.

### Example 45

### 5-Bromobenzo[d]thiazol-2-amine (Intermediate 23)

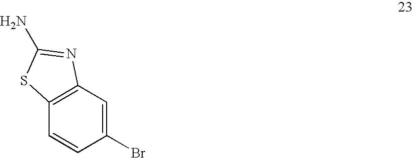

The 4-bromo-2-nitrobenzenamine (4.34 g, 20 mmol) was suspended in 10% H₂SO₄ (100 mL) and cooled in ice-H₂O. The solution of NaNO₂ (2.08 g, 30 mmol) in H₂O (5 mL) was added potionally. The mixture was stirred for 4 h at 0˜5° C. The suspension of CuSCN (1.0 g, 8.2 mmol) in H₂O and aqueous solution of KSCN (2.91 g, 30 mmol in 10 mL H₂O) were added. The gas was generated and mixture was stirred for 0.5 h in ice-H₂O, then 0.5 h at room temperature. The reaction was heated under reflux for 1 h. The solid was collected and washed with H₂O. The solid was suspended in 10% HCl (100 mL) followed by adding SnCl₂ (8.0 g, 42 mmol). The mixture was stirred for 10 h at room temperature and heated under reflux for 3 h. The solid was collected and washed with H₂O and air dried to afforded The title intermediate (2.6 g, 57%) as yellow solid.

¹H NMR (500 MHz, DMSO-d₆): δ 7.17 (dd, J=1.3 Hz, J=8.4 Hz, 1H), 7.50 (s, 1H), 7.63 (d, J=8.4 Hz, 1H), 7.90 (br s, 2H). MS (ES+): m/z 229 (M+H)⁺.

### Example 46

### 5-((E)-2-(2-(4-(Piperidin-4-ylsulfonyl)phenylamino)pyrimidin-5-yl)vinyl)benzo[d]thiazol-2-amine (Compound XXII)

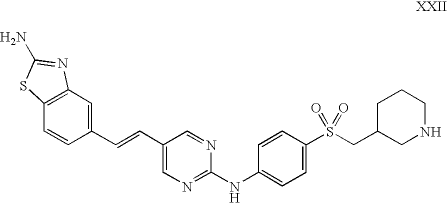

To a solution of intermediate 6 (444 mg, 1.0 mmol) in DMF (10 mL) was added 23 (229 mg, 1.0 mmol), Pd(OAc)₂ (45 mg, 0.2 mmol), and Et₃N (0.56 mL, 4.0 mmol). The reaction was heated at 180° C. for 60 min in microwave. The solid was filtered off and washed with EtOAc. The filtrate was washed with brine (1×50 mL). The organic solution was separated. The aqueous was extracted with EtOAc (2×20 mL). The combined organic phase was dried (Na₂SO₄) and concentrated. The residue was dissolved in anhydrous CH₂Cl₂ (10 mL) and the TFA (2 mL) was added. The reaction was stirred for 4 h at room temperature. The 10% NaOH (20 mL) was added. The organic layer was separated and aqueous was extracted with CH₂Cl₂ (2×10 mL). The combined organic layer was dried (Na₂SO₄) and the solvent was removed in vacuo. The residue was purified by HPLC to afford the title compound (20 mg of HCl salt, 4%) as a yellow solid.

¹H NMR (500 MHz, DMSO-d₆): δ 1.66-1.74 (m, 2H), 2.00-2.03 (m, 2H), 2.82-2.88 (m, 2H), 3.31-3.34 (m, 2H), 3.46-3.51 (m, 1H), 7.21 (d, J=16.5 Hz, 1H), 7.42 (d, J=16.5 Hz, 1H), 7.43 (d, J=8.3 Hz, 1H), 7.61 (s, 1H), 7.76 (d, J=8.8 Hz, 2H), 7.79 (d, J=8.3 Hz, 1H), 8.08 (d, J=8.8 Hz, 2H), 8.54 (br s, 1H), 8.88 (s, 2H), 9.05 (br s, 1H), 10.47 (s, 1H). MS (ES+): m/z 493 (M+H)⁺.

### Example 47

### tert-Butyl 4-(4-bromophenylsulfonyl)piperazine-1-carboxylate (Intermediate 24)

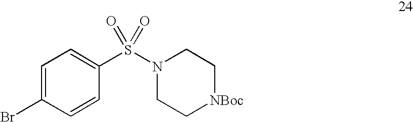

Triethylamine (2.1 mL, 15.0 mmol) was added to a solution of tert-butyl piperazine-1-carboxylate (0.93 g, 5.0 mmol) and 4-bromobenzenesulfonyl chloride (1.28 g, 5.0 mmol) dissolved in DCM (20 mL). After stirring 14 h at room temperature, the solution was diluted with EtOAc (20 mL), washed two times with saturated aqueous NaHCO₃ (2×10 mL), and washed with brine. The solution was dried over MgSO₄ and concentrated in vacuo to afford the title intermediate as a white solid (1.92 g, 95%).

¹H NMR (500 MHz, DMSO-d₆): δ 1.41 (s, 9H), 2.97 (t, J=4.9 Hz, 4H), 3.51 (t, J=5.0 Hz, 4H), 7.61 (d, J=8.9 Hz, 2H), 7.69 (d, J=8.9 Hz, 2H).

### Example 48

### tert-Butyl 4-(4-(5-vinylpyrimidin-2-ylamino)phenylsulfonyl)piperazine-1-carboxylate (Intermediate 25)

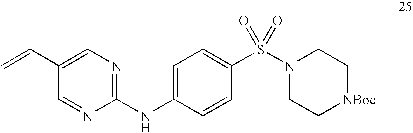

A suspension of intermediate 24 (67 mg, 0.17 mmol), intermediate 1 (20 mg, 0.17 mmol), Pd(OAc)₂, Xantphos (9.6 mg, 0.017 mmol), and potassium tert-butoxide (41 mg, 0.36 mmol) in 1,4-dioxane (1.8 mL) and DMF (0.1 mL) was purged 5 min with argon gas. The suspension was sealed in a microwave reaction tube and irradiated with microwave 20 min at 160° C. After cooling to room temperature, the cap was removed and the resulting mixture was filtered through a pad of silica gel and the filtered solid was washed with EtOAc. The filtrate was concentrated and the residue was purified by silica gel chromatography (hexanes/EtOAc 10:0 to 6:4 gradient) to afford the title intermediate as a tan solid (31 mg, 42%).

¹H NMR (500 MHz, DMSO-d₆): δ 1.33 (s, 9H), 2.81 (t, J=4.9 Hz, 4H), 3.38 (br s, 4H), 5.30 (d, J=11.3 Hz, 1H), 5.94 (d, J=17.9 Hz, 1H), 6.66 (dd, J=17.8, 11.2 Hz, 1H), 7.65 (d, J=8.9 Hz, 2H), 8.03 (d, J=8.9 Hz, 2H), 8.72 (s, 2H). MS (ES+): m/z 446 (M+H)⁺.

### Example 49

### (E)-6-(2-(2-(4-(piperazin-1-ylsulfonyl)phenylamino)pyrimidin-5-yl)vinyl)benzo[d]thiazol-2-amine hydrochloride (Compound XXIII)

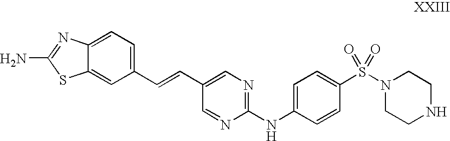

To a suspension of intermediate 25 (0.40 g, 0.90 mmol), 2-amino-6-bromobenzothiazole (0.41 g, 1.80 mmol), Pd₂(dba)₃ (82 mg, 0.09 mmol) and cesium carbonate (0.59 g, 1.80 mmol) in 1,4-dioxane (12 mL) was added a solution of 1 M tri-tert-butylphosphine in toluene (0.36 mL, 0.36 mmol). The suspension was purged 5 min with argon, sealed in a microwave reaction tube and irradiated with microwave 30 min at 180° C. After cooling to room temperature, the cap was removed and the resulting mixture was filtered through a pad of silica gel and the filtered solid was washed with EtOAc. The filtrate was concentrated and the residue was purified by silica gel chromatography (hexanes/EtOAc 10:0 to 3:7 gradient) to give a yellow solid. The yellow solid was dissolved in DCM (10 mL), treated with trifluoroacetic acid (1 mL), and stirred overnight at room temperature. The reaction mixture was purified by HPLC. Fractions that contained The title compound were combined and neutralized with aqueous NaHCO₃ and extracted with EtOAc. The organic layer was treated with 4 M HCl in 1,4-dioxane (0.2 mL) and concentrated in vacuo to afford the title compound as a yellow solid (154 mg).

¹H NMR (500 MHz, DMSO-d₆): δ 3.12 (m, 4H), 3.16 (m, 4H), 4.12 (d, J=16.6 Hz, 1H), 7.36 (d, J=16.6 Hz, 1H), 7.45 (d, J=8.4 Hz, 1H), 7.57 (dd, J=8.5, 1.4 Hz, 1H), 7.70 (d, J=9.0 Hz, 1H), 8.01 (d, J=1.2 Hz, 1H), 8.07 (d, J=8.9 Hz, 1H), 8.84 (s, 2H), 8.90-9.20 (br s, 1H), 9.09 (br s, 3H), 10.42 (s, 1H). MS (ES+): m/z 494 (M+H)⁺.

### Example 50

### 1-(4-Bromophenylsulfonyl)piperidin-4-ol (Intermediate 26)

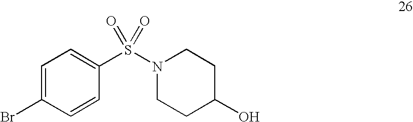

Triethylamine (2.1 mL, 15.0 mmol) was added to a solution of 4-hydroxypiperidine (0.51 g, 5.0 mmol) and 4-bromobenzenesulfonyl chloride (1.28 g, 5.0 mmol) dissolved in DCM (20 mL). After stirring 14 h at room temperature, the solution was diluted with EtOAc (20 mL), washed two times with saturated aqueous NaHCO₃ (2×10 mL), and washed with brine. The solution was dried over MgSO₄ and concentrated in vacuo to afford the title intermediate as an off-white solid (1.44 g, 90%).

¹H NMR (500 MHz, DMSO-d₆): δ 1.38-1.44 (m, 2H), 1.70-1.75 (m, 2H), 2.73 (ddd, J=11.6, 8.6, 3.3 Hz, 2H), 3.14 (ddd, J=15.2, 6.8, 3.8 Hz, 2H), 3.51-3.55 (m, 1H), 4.68 (d, J=3.9 Hz, 1H), 7.65 (d, J=9.2 Hz, 2H), 7.86 (d, J=9.4 Hz, 2H). MS (ES+): m/z 322 (M+H)⁺.

### Example 51

### 1-(4-(5-Vinylpyrimidin-2-ylamino)phenylsulfonyl)piperidin-4-ol (Intermediate 27)

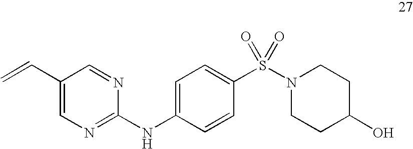

A suspension of intermediate 26 (0.53 g, 1.65 mmol), intermediate 1 (0.20 g, 1.65 mmol), Pd(OAc)₂ (18.5 mg, 0.083 mmol), Xantphos (9.6 mg, 0.165 mmol), and potassium tert-butoxide (0.41 g, 3.63 mmol) in 1,4-dioxane (12 mL) and DMF (3 mL) was purged 5 min with argon gas. The suspension was sealed in a microwave reaction tube and irradiated with microwave 30 min at 180° C. After cooling to room temperature, the cap was removed and the resulting mixture was filtered through a pad of silica gel and the filtered solid was washed with EtOAc. The filtrate was concentrated and the residue was purified by silica gel chromatography (hexanes/EtOAc 10:0 to 4:6 gradient) to afford the title intermediate as a tan solid (0.25 g, 43%).

¹H NMR (500 MHz, DMSO-d₆): δ 1.38-1.45 (m, 2H), 1.70-1.74 (m, 2H), 2.64-2.68 (m, 2H), 3.10-3.17 (m, 2H), 3.49-3.51 (m, 1H), 4.65 (d, J=3.9 Hz, 1H), 5.29 (d, J=11.6 Hz, 1H), 5.93 (d, J=17.7 Hz, 1H), 6.66 (dd, J=17.9, 11.2 Hz, 1H), 7.64 (d, J=9.5 Hz, 2H), 8.01 (d, J=9.5 Hz, 2H), 8.72 (s, 2H), 10.30 (s, 1H). MS (ES+): m/z 361 (M+H)⁺.

### Example 52

### (E)-1-(4-(5-(2-(2-Aminobenzo[d]thiazol-6-yl)vinyl)pyrimidin-2-ylamino)phenylsulfonyl)piperidin-4-ol 2,2,2-trifluoroacetate (Compound XXIV)

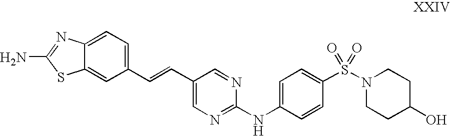

To a suspension of intermediate 27 (75 mg, 0.21 mmol), 2-amino-6-bromobenzothiazole (96 mg, 0.42 mmol), Pd₂(dba)₃ (19 mg, 0.021 mmol), and cesium carbonate (0.14 g, 0.42 mmol) in 1,4-dioxane (3 mL) was added a solution of 1 M tri-tert-butylphosphine in toluene (0.083 mL, 0.084 mmol). The suspension was purged 5 min with argon, sealed in a microwave reaction tube and irradiated with microwave 60 min at 200° C. After cooling to room temperature, the cap was removed and the resulting mixture was filtered through a pad of silica gel and the filtered solid was washed with EtOAc. The filtrate was concentrated and the residue was purified by HPLC to afford the title compound as a yellow solid (19 mg).

¹H NMR (500 MHz, DMSO-d₆): δ 1.40-1.45 (m, 2H), 1.71-1.75 (m, 2H), 2.66-2.69 (m, 2H), 3.10-3.15 (m, 2H), 3.49-3.52 (m, 1H), 7.08 (d, J=16.5 Hz, 1H), 7.33 (d, J=16.5 Hz, 1H), 7.37 (d, J=8.4 Hz, 1H), 7.50 (dd, J=8.6, 1.7 Hz, 1H), 7.65 (d, J=9.0 Hz, 1H), 7.94 (d, J=1.4 Hz, 1H), 8.02 (d, J=9.0 Hz, 1H), 8.15 (br s, 2H), 8.81 (s, 2H), 10.32 (s, 1H). MS (ES+): m/z 509 (M+H)⁺.

### Example 53

### 1-(4-Bromophenylsulfonyl)piperidin-3-ol (Intermediate 28)

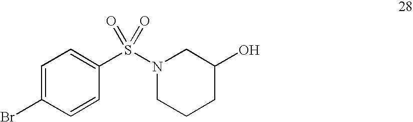

Triethylamine (4.2 mL, 30.0 mmol) was added to a solution of 3-hydroxypiperidine (1.01 g, 10.0 mmol) and 4-bromobenzenesulfonyl chloride (2.56 g, 10.0 mmol) dissolved in DCM (40 mL). After stirring overnight at room temperature, the solution was diluted with EtOAc (40 mL), washed two times with saturated aqueous NaHCO₃ (2×20 mL), and washed with brine. The solution was dried over MgSO₄ and concentrated in vacuo to afford the title intermediate as a light yellow solid (3.12 g, 98%).

¹H NMR (500 MHz, DMSO-d₆): δ 1.08-1.15 (m, 1H), 1.38-1.45 (m, 1H), 1.67-1.72 (m, 1H), 2.17-2.21 (dd, J=10.9, 8.7 Hz, 1H), 2.38 (t, J=13.1 Hz, 1H), 3.26 (m, 1H), 3.36-3.36 (dd, J=11.1, 4.0 Hz, 1H), 3.53 (m, 1H), 4.98 (d, J=4.5 Hz, 1H), 7.66 (d, J=8.7 Hz, 1H), 7.86 (d, J=8.5 Hz, 1H). MS (ES+): m/z 304 (M+H)⁺.

### Example 54

### 1-(4-(5-Vinylpyrimidin-2-ylamino)phenylsulfonyl)piperidin-3-ol (Intermediate 29)

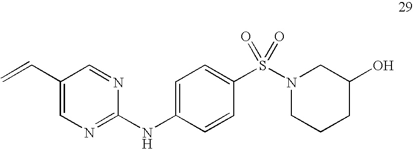

A suspension of intermediate 28 (0.53 g, 1.65 mmol), intermediate 1 (0.20 g, 1.65 mmol), Pd(OAc)₂ (18.5 mg, 0.083 mmol), Xantphos (9.6 mg, 0.165 mmol), and potassium tert-butoxide (0.41 g, 3.63 mmol) in 1,4-dioxane (12 mL) and DMF (3 mL) was purged 5 min with argon gas. The suspension was sealed in a microwave reaction tube and irradiated with microwave 30 min at 180° C. After cooling to room temperature, the cap was removed and the resulting mixture was filtered through a pad of silica gel and the filtered solid was washed with EtOAc. The filtrate was concentrated and the residue was purified by silica gel chromatography (hexanes/EtOAc 10:0 to 4:6 gradient) to afford the title intermediate as a tan solid (0.22 g, 37%).

¹H NMR (500 MHz, DMSO-d₆): δ 1.05-1.09 (m, 1H), 1.42-1.44 (m, 1H), 1.68-1.73 (m, 2H), 2.08 (t, J=10.4 Hz, 1H), 2.29 (t, J=9.0 Hz, 1H), 3.26-3.31 (m, 1H), 3.40 (d, J=10.3 Hz, 1H), 3.49-3.54 (m, 1H), 4.94 (d, J=4.6 Hz, 1H), 5.28 (d, J=11.3 Hz, 1H), 5.92 (d, J=17.9 Hz, 1H), 6.66 (dd, J=17.8, 11.2 Hz, 1H), 7.64 (d, J=8.8 Hz, 2H), 8.01 (d, J=8.8 Hz, 2H), 8.72 (s, 2H), 10.31 (s, 1H). MS (ES+): m/z 361 (M+H)⁺.

### Example 55

### (E)-1-(4-(5-(2-(2-Aminobenzo[d]thiazol-6-yl)vinyl)pyrimidin-2-ylamino)phenylsulfonyl)piperidin-3-ol hydrochloride (Compound XXV)

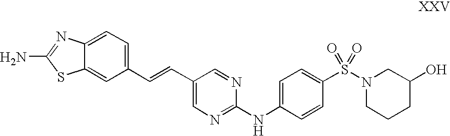

To a suspension of intermediate 30 (75 mg, 0.21 mmol), 2-amino-6-bromobenzothiazole (96 mg, 0.42 mmol), Pd₂(dba)₃ (19 mg, 0.021 mmol), and cesium carbonate (0.14 g, 0.42 mmol) in 1,4-dioxane (3 mL) was added a solution of 1 M tri-tert-butylphosphine in toluene (0.083 mL, 0.084 mmol). The suspension was purged 5 min with argon, sealed in a microwave reaction tube and irradiated with microwave 60 min at 200° C. After cooling to room temperature, the cap was removed and the resulting mixture was filtered through a pad of silica gel and the filtered solid was washed with EtOAc. The filtrate was concentrated and the residue was purified by HPLC. Fractions that contained The title compound were combined and neutralized with aqueous NaHCO₃ and extracted with EtOAc. The organic layer was treated with 4 M HCl in 1,4-dioxane (0.2 mL) and concentrated in vacuo to afford the title compound as a yellow solid (33 mg).

¹H NMR (500 MHz, DMSO-d₆): δ 1.05-1.09 (m, 1H), 1.42-1.46 (m, 1H), 1.69-1.73 (m, 1H), 2.09 (t, J=10.9 Hz, 1H), 2.29 (t, J=11.2 Hz, 1H), 3.26-3.33 (m, 1H), 3.38-3.42 (m, 1H), 3.49-3.54 (m, 1H), 4.95 (d, J=4.8 Hz, 1H), 7.07 (d, J=16.5 Hz, 1H), 7.31 (d, J=16.9 Hz, 1H), 7.32 (d, J=8.2 Hz, 1H), 7.44 (dd, J=8.5, 1.6 Hz, 1H), 7.57 (s, 2H), 7.64 (d, J=8.9 Hz, 2H), 7.88 (d, J=1.6 Hz, 1H), 8.02 (d, J=8.9 Hz, 2H), 8.80 (s, 2H), 10.31 (s, 1H). MS (ES+): m/z 509 (M+H)⁺.

### Example 56

### tert-Butyl 4-(4-bromophenylsulfonyl)-1,4-diazepane-1-carboxylate (Intermediate 30)

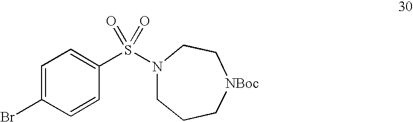

Triethylamine (4.2 mL, 30.0 mmol) was added to a solution of 1-Boc-hexahydro-1,4-diazepine (2.00 g, 10.0 mmol) and 4-bromobenzenesulfonyl chloride (2.56 g, 10.0 mmol) dissolved in DCM (40 mL). After stirring overnight at room temperature, the solution was diluted with EtOAc (40 mL), washed two times with saturated aqueous NaHCO₃ (2×20 mL), and washed with brine. The solution was dried over MgSO₄ and concentrated in vacuo to afford the title intermediate as colorless oil (3.07 g, 73%).

¹H NMR (500 MHz, DMSO-d₆): δ 1.38 (s, 9H), 1.67-1.73 (m, 2H), 3.18-3.23 (m, 2H), 3.30-3.36 (m, 2H), 3.38-3.43 (m, 2H), 7.72 (d, J=7.9 Hz, 2H), 7.81 (d, J=7.9 Hz, 2H). MS (ES+): m/z 421 (M+H)⁺.

### Example 57

### tert-Butyl 4-(4-(5-vinylpyrimidin-2-ylamino)phenylsulfonyl)-1,4-diazepane-1-carboxylate (Intermediate 31)

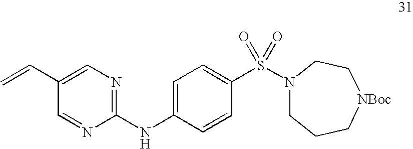

A suspension of intermediate 30 (0.69 g, 1.65 mmol), intermediate 1 (0.20 g, 1.65 mmol), Pd(OAc)₂ (18.5 mg, 0.083 mmol), Xantphos (9.6 mg, 0.165 mmol), and potassium tert-butoxide (0.41 g, 3.63 mmol) in 1,4-dioxane (12 mL) and DMF (3 mL) was purged 5 min with argon gas. The suspension was sealed in a microwave reaction tube and irradiated with microwave 30 min at 180° C. After cooling to room temperature, the cap was removed and the resulting mixture was filtered through a pad of silica gel and the filtered solid was washed with EtOAc. The filtrate was concentrated and the residue was purified by silica gel chromatography (hexanes/EtOAc 10:0 to 7:3 gradient) to afford the title intermediate as light yellow oil (0.30 g, 40%).

¹H NMR (500 MHz, DMSO-d₆): δ 1.37 (s, 9H), 1.66-1.74 (m, 2H), 3.12-3.18 (m, 2H), 3.21-3.26 (m, 2H), 3.30-3.36 (m, 2H), 3.37-3.43 (m, 2H), 5.28 (d, J=11.6 Hz, 1H), 5.91 (d, J=17.8 Hz, 1H), 6.65 (dd, J=17.8, 11.3 Hz, 1H), 7.69 (d, J=8.9 Hz, 1H), 7.97 (d, J=9.0 Hz, 1H), 8.71 (s, 2H), 10.27 (s, 1H). MS (ES+): m/z 460 (M+H)⁺.

### Example 58

### (E)-6-(2-(2-(4-(1,4-Diazepan-1-ylsulfonyl)phenylamino)pyrimidin-5-yl)vinyl)benzo[d]thiazol-2-amine hydrochloride (Compound XXVI)

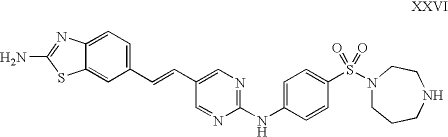

To a suspension of intermediate 32 (0.50 g, 1.09 mmol), 2-amino-6-bromobenzothiazole (0.50 g, 2.18 mmol), Pd₂(dba)₃ (0.10 g, 0.11 mmol) and cesium carbonate (0.71 g, 2.18 mmol) in 1,4-dioxane (15 mL) was added a solution of 1 M tri-tert-butylphosphine in toluene (0.44 mL, 0.44 mmol). The suspension was purged 5 min with argon, sealed in a microwave reaction tube and irradiated with microwave 60 min at 180° C. After cooling to room temperature, the cap was removed and the resulting mixture was filtered through a pad of silica gel and the filtered solid was washed with EtOAc. The filtrate was concentrated and the residue was purified by silica gel chromatography (hexanes/EtOAc 10:0 to 3:7 gradient) to give a yellow solid. The yellow solid was dissolved in DCM (20 mL), treated with trifluoroacetic acid (3 mL), and stirred overnight at room temperature. The reaction mixture was purified by HPLC. Fractions that contained The title compound were combined and neutralized with aqueous NaHCO₃ and extracted with EtOAc. The organic layer was treated with 4 M HCl in 1,4-dioxane (0.2 mL) and concentrated in vacuo to afford the title compound as a yellow solid (0.22 g).

¹H NMR (500 MHz, DMSO-d₆): δ 1.95-2.01 (m, 2H), 3.09-3.17 (m, 4H), 3.28 (t, J=6.0 Hz, 2H), 3.48-3.50 (m, 2H), 7.12 (d, J=16.6 Hz, 1H), 7.36 (d, J=16.6 Hz, 1H), 7.47 (d, J=8.4 Hz, 1H), 7.58 (d, J=8.3 Hz, 1H), 7.73 (d, J=8.9 Hz, 1H), 8.02 (s, 1H), 8.03 (d, J=8.9 Hz, 1H), 8.83 (s, 2H), 9.05-9.35 (br s, 2H), 9.40-9.46 (br s, 2H), 10.36 (s, 1H). MS (ES+): m/z 508 (M+H)⁺.

### Example 59

### tert-Butyl 4-(4-bromo-2-fluorophenylsulfonyl)-1,4-diazepane-1-carboxylate (Intermediate 32)

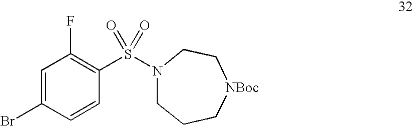

Triethylamine (2.1 mL, 15.0 mmol) was added to a solution of 1-Boc-hexahydro-1,4-diazepine (1.00 g, 5.0 mmol) and 4-bromo-2-fluorobenzenesulfonyl chloride (1.37 g, 5.0 mmol) dissolved in DCM (20 mL). After stirring overnight at room temperature, the solution was diluted with EtOAc (40 mL), washed two times with saturated aqueous NaHCO₃ (2×15 mL), and washed with brine. The solution was dried over MgSO₄ and concentrated in vacuo to afford the title intermediate as pale yellow oil (2.14 g, 98%).

¹H NMR (500 MHz, DMSO-d₆): δ 1.69-1.77 (m, 2H), 3.27-3.31 (m, 2H), 3.33-3.39 (m, 4H), 3.40-3.45 (m, 2H), 7.63 (d, J=8.4 Hz, 1H), 7.74 (t, J=8.0 Hz, 1H), 7.88 (dd, J=10.0, 3.6 Hz, 1H). ¹⁹F NMR (470 MHz, DMSO-d₆): δ −105.91 (d, J=14.1 Hz, 1F). MS (ES+): m/z 459 (M+Na)⁺.

### Example 60

### tert-Butyl 4-(2-fluoro-4-(5-vinylpyrimidin-2-ylamino)phenylsulfonyl)-1,4-diazepane-1-carboxylate (intermediate 33)

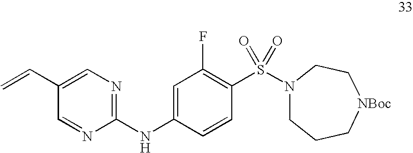

A suspension of intermediate 32 (1.08 g, 2.47 mmol), intermediate 1 (0.30 g, 2.47 mmol), Pd(OAc)₂ (28 mg, 0.12 mmol), Xantphos (0.14 g, 0.25 mmol), and potassium tert-butoxide (0.61 g, 5.44 mmol) in 1,4-dioxane (12 mL) and DMF (3 mL) was purged 5 min with argon gas. The suspension was sealed in a microwave reaction tube and irradiated with microwave 30 min at 180° C. After cooling to room temperature, the cap was removed and the resulting mixture was filtered through a pad of silica gel and the filtered solid was washed with EtOAc. The filtrate was concentrated and the residue was purified by silica gel chromatography (hexanes/EtOAc 10:0 to 6:4 gradient) to afford the title intermediate as light yellow oil (0.56 g, 47%).

¹H NMR (500 MHz, DMSO-d₆): δ 1.38 (s, 9H), 1.67-1.74 (m, 2H), 3.16-3.45 (m, 8H), 5.30 (d, J=11.4 Hz, 1H), 5.93 (d, J=17.9 Hz, 1H), 6.65 (dd, J=17.9, 11.2 Hz, 1H), 7.57-7.69 (m, 2H), 8.02 (dd, J=14.1, 2.0 Hz, 1H), 8.75 (s, 2H), 10.48 (s, 1H). ¹⁹F NMR (470 MHz, DMSO-d₆): δ −107.21 (d, J=14.1 Hz, 1F). MS (ES+): m/z 478 (M+H)⁺.

### Example 61

### (E)-6-(2-(2-(4-(1,4-Diazepan-1-ylsulfonyl)-3-fluorophenylamino)pyrimidin-5-yl)vinyl)benzo[d]thiazol-2-amine (Compound XXVII)

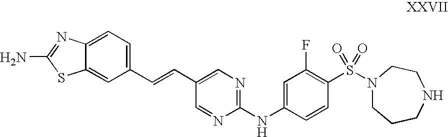

To a suspension of intermediate 33 (0.55 g, 1.15 mmol), 2-amino-6-bromobenzothiazole (0.53 g, 2.30 mmol), Pd₂(dba)₃ (0.11 mg, 0.12 mmol) and cesium carbonate (0.75 g, 2.30 mmol) in 1,4-dioxane (15 mL) was added a solution of 1 M tri-tert-butylphosphine in toluene (0.46 mL, 0.46 mmol). The suspension was purged 5 min with argon, sealed in a microwave reaction tube and irradiated with microwave 60 min at 180° C. After cooling to room temperature, the cap was removed and the resulting mixture was filtered through a pad of silica gel and the filtered solid was washed with EtOAc. The filtrate was concentrated and the residue was purified by silica gel chromatography (hexanes/EtOAc 10:0 to 3:7 gradient) to give a yellow solid. The yellow solid was dissolved in DCM (4 mL), treated with 4 M HCl in dioxane (4 mL), and stirred 5 min at room temperature. The reaction mixture was purified by HPLC. Fractions that contained The title compound were combined and neutralized with aqueous NaHCO₃ and extracted with EtOAc. The organic layer was concentrated in vacuo to afford the title compound as a yellow solid (10 mg).

¹H NMR (500 MHz, DMSO-d₆): δ 1.68 (dddd, J=6.0, 6.0, 5.8, 5.8 Hz, 2H), 2.76 (t, J=5.9 Hz, 2H), 2.80 (m, 2H), 3.26-3.40 (m, 4H), 7.07 (d, J=16.6 Hz, 1H), 7.32 (d, J=16.2 Hz, 1H), 7.33 (s, 1H), 7.44 (dd, J=8.4, 1.7 Hz, 1H), 7.58 (s, 2H), 7.62 (dd, J=8.9, 1.8 Hz, 1H), 7.65 (t, J=8.7 Hz, 1H), 7.88 (s, 1H), 8.04 (dd, J=14.1, 1.9 Hz, 1H), 8.83 (s, 2H), 10.47 (s, 1H). ¹⁹F NMR (470 MHz, DMSO-d₆): δ −107.05 (s, 1F). MS (ES+): m/z 526 (M+H)⁺.

### Example 62

### tert-Butyl 4-(4-bromo-2-methylphenylsulfonyl)-1,4-diazepane-1-carboxylate (Intermediate 34)

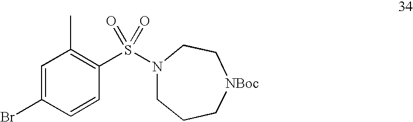

Triethylamine (2.1 mL, 15.0 mmol) was added to a solution of 1-Boc-hexahydro-1,4-diazepine (1.00 g, 5.0 mmol) and 4-bromo-2-methylbenzenesulfonyl chloride (1.35 g, 5.0 mmol) dissolved in DCM (20 mL). After stirring overnight at room temperature, the solution was diluted with EtOAc (40 mL), washed two times with saturated aqueous NaHCO₃ (2×15 mL), and washed with brine. The solution was dried over MgSO₄ and concentrated in vacuo to afford the title intermediate as pale yellow oil (2.14 g, 98%).

¹H NMR (500 MHz, DMSO-d₆): δ 1.38 and 1.39 (s and s, 9H, Boc group slow rotation), 1.67-1.78 (m, 2H), 3.26-3.32 (m, 2H), 3.33-3.40 (m, 4H), 3.40-3.46 (m, 2H), 7.58-7.60 (m, 1H), 7.69 (dd, J=8.3, 4.0 Hz, 1H), 7.71 (s, 1H). The CH₃ group is apparently overlapped with the DMSO signal and hence, not reported here. A change in chemical shift was observed in the ensuring intermediate. MS (ES+): m/z 335 (M-Boc+H)⁺.

### Example 63

### tert-Butyl 4-(2-methyl-4-(5-vinylpyrimidin-2-ylamino)phenylsulfonyl)-1,4-diazepane-1-carboxylate (Intermediate 35)

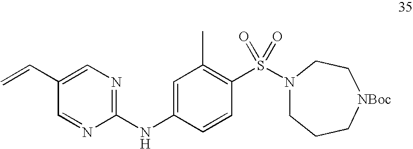

A suspension of intermediate 34 (2.00 g, 4.62 mmol), intermediate 1 (0.56 g, 4.62 mmol), Pd(OAc)₂ (52 mg, 0.23 mmol), Xantphos (0.27 g, 0.46 mmol), and potassium tert-butoxide (1.14 g, 10.2 mmol) in 1,4-dioxane (12 mL) and DMF (3 mL) was purged 5 min with argon gas. The suspension was sealed in a microwave reaction tube and irradiated with microwave 30 min at 180° C. After cooling to room temperature, the cap was removed and the resulting mixture was filtered through a pad of silica gel and the filtered solid was washed with EtOAc. The filtrate was concentrated and the residue was purified by silica gel chromatography (hexanes/EtOAc 10:0 to 6:4 gradient) to afford the title intermediate as light orange oil (0.67 g, 31%).

¹H NMR (500 MHz, DMSO-d₆): δ 1.38 and 1.39 (s and s, 9H, Boc group slow rotation), 1.65-1.75 (m, 2H), 2.46 (s, 3H), 3.24-3.29 (m, 2H), 3.31-3.34 (m, 2H), 3.35-3.42 (m, 4H), 5.27 (d, J=11.5 Hz, 1H), 5.90 (d, J=17.7 Hz, 1H), 6.65 (dd, J=17.8, 11.2 Hz, 1H), 7.71 (dd, J=8.7, 3.5 Hz, 1H), 7.78 (d, J=6.1 Hz, 1H), 7.82 (dd, J=8.8, 2.3 Hz, 1H), 8.71 (s, 2H), 10.17 (s, 1H). MS (ES+): m/z 474 (M+H)⁺.

### Example 64

### (E)-6-(2-(2-(4-(1,4-Diazepan-1-ylsulfonyl)-3-methylphenylamino)pyrimidin-5-yl)vinyl)benzo[d]thiazol-2-amine hydrochloride (Compound XXVIII)

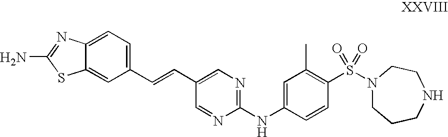

To a suspension of intermediate 35 (0.40 g, 0.84 mmol), 2-amino-6-bromobenzothiazole (0.39 g, 1.70 mmol), Pd₂(dba)₃ (77 mg, 0.08 mmol) and cesium carbonate (0.55 g, 1.70 mmol) in 1,4-dioxane (12 mL) was added a solution of 1 M tri-tert-butylphosphine in toluene (0.34 mL, 0.34 mmol). The suspension was purged 5 min with argon, sealed in a microwave reaction tube and irradiated with microwave 60 min at 180° C. After cooling to room temperature, the cap was removed and the resulting mixture was filtered through a pad of silica gel and the filtered solid was washed with EtOAc. The filtrate was concentrated and the residue was purified by silica gel chromatography (hexanes/EtOAc 10:0 to 3:7 gradient) to give a yellow solid. The yellow solid was dissolved in DCM (6 mL), treated with trifluoroacetic acid (1 mL), and stirred overnight at room temperature. The reaction mixture was purified by HPLC. Fractions that contained The title compound were combined and neutralized with aqueous NaHCO₃ and extracted with EtOAc. The organic layer was treated with 4 M HCl in 1,4-dioxane (0.2 mL) and concentrated in vacuo to afford the title compound as a yellow solid (68 mg).

¹H NMR (500 MHz, DMSO-d₆): δ 2.06 (m, 2H), 2.80-3.20 (br s, 2H), 3.15 (m, 4H), 3.37 (t, J=6.1 Hz, 1H), 3.43 (dddd, J=7.2, 7.2, 7.0, 7.0 Hz, 2H), 7.09 (d, J=16.5 Hz, 1H), 7.33 (d, J=16.5 Hz, 1H), 7.42 (d, J=8.3 Hz, 1H), 7.52 (d, J=9.5 Hz, 1H), 7.72 (d, J=8.9 Hz, 1H), 7.87 (d, J=10.8 Hz, 1H), 7.96 (s, 1H), 8.81 (s, 2H), 9.65-9.71 (br s, 2H), 10.24 (s, 1H). The CH₃ group is apparently overlapped with the DMSO signal and hence, not reported here. MS (ES+): m/z 522 (M+H)⁺.

### Example 65

### tert-Butyl 4-(4-bromo-3-fluorophenylsulfonyl)-1,4-diazepane-1-carboxylate (Intermediate 36)

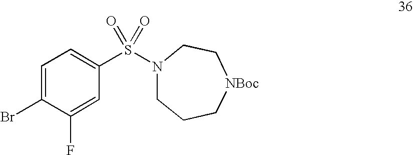

Triethylamine (2.1 mL, 15.0 mmol) was added to a solution of 1-Boc-hexahydro-1,4-diazepine (1.00 g, 5.0 mmol) and 4-bromo-3-fluorobenzenesulfonyl chloride (1.37 g, 5.0 mmol) dissolved in DCM (20 mL). After stirring overnight at room temperature, the solution was diluted with EtOAc (40 mL), washed two times with saturated aqueous NaHCO₃ (2×15 mL), and washed with brine. The solution was dried over MgSO₄ and concentrated in vacuo to afford the title intermediate as pale yellow oil (2.14 g, 98%).

¹H NMR (500 MHz, DMSO-d₆): δ 1.37 and 1.39 (s and s, 9H, Boc group slow rotation), 1.66-1.75 (m, 2H), 3.21-3.27 (m, 2H), 3.29-3.34 (m, 4H), 3.40-3.43 (m, 2H), 7.57 (d, J=8.3 Hz, 1H), 7.79-7.81 (m, 1H), 7.96 (td, J=5.9, 2.5 Hz, 1H). MS (ES+): m/z 339 (M-Boc+H)⁺.

### Example 66

### tert-Butyl 4-(3-fluoro-4-(5-vinylpyrimidin-2-ylamino)phenylsulfonyl)-1,4-diazepane-1-carboxylate (Intermediate 37)

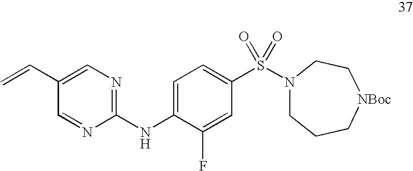

A suspension of intermediate 36 (2.00 g, 4.57 mmol), intermediate 1 (0.56 g, 4.57 mmol), Pd(OAc)₂ (51 mg, 0.23 mmol), Xantphos (0.27 g, 0.46 mmol), and potassium tert-butoxide (1.13 g, 10.1 mmol) in 1,4-dioxane (12 mL) and DMF (3 mL) was purged 5 min with argon gas. The suspension was sealed in a microwave reaction tube and irradiated with microwave 30 min at 180° C. After cooling to room temperature, the cap was removed and the resulting mixture was filtered through a pad of silica gel and the filtered solid was washed with EtOAc. The filtrate was concentrated and the residue was purified by silica gel chromatography (hexanes/EtOAc 10:0 to 6:4 gradient) to afford the title intermediate as a white solid (0.38 g, 17%).

¹H NMR (500 MHz, DMSO-d₆): δ 1.37 and 1.38 (s and s, 9H, Boc group slow rotation), 1.67-1.73 (m, 2H), 3.18-3.24 (m, 2H), 3.25-3.35 (m, 4H), 3.40-3.44 (m, 2H), 5.28 (d, J=11.6 Hz, 1H), 5.90 (d, J=18.0 Hz, 1H), 6.65 (dd, J=17.8, 11.3 Hz, 1H), 7.60 (d, J=8.7 Hz, 1H), 7.64 (d, J=10.5 Hz, 1H), 8.25 (t, J=8.6 Hz, 1H), 8.68 (s, 2H), 9.67 (s, 1H). MS (ES+): m/z 478 (M+H)⁺.

### Example 67

### (E)-6-(2-(2-(4-(1,4-Diazepan-1-ylsulfonyl)-2-fluorophenylamino)pyrimidin-5-yl)vinyl)benzo[d]thiazol-2-amine (Compound XXIX)

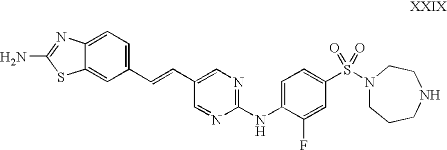

To a suspension of intermediate 37 (0.36 g, 0.79 mmol), 2-amino-6-bromobenzothiazole (0.36 g, 1.57 mmol), Pd₂(dba)₃ (72 mg, 0.08 mmol) and cesium carbonate (0.51 g, 1.57 mmol) in 1,4-dioxane (9 mL) was added a solution of 1 M tri-tert-butylphosphine in toluene (0.31 mL, 0.31 mmol). The suspension was purged 5 min with argon, sealed in a microwave reaction tube and irradiated with microwave 60 min at 180° C. After cooling to room temperature, the cap was removed and the resulting mixture was filtered through a pad of silica gel and the filtered solid was washed with EtOAc. The filtrate was concentrated and the residue was purified by silica gel chromatography (hexanes/EtOAc 10:0 to 3:7 gradient) to give a yellow solid. The yellow solid was dissolved in DCM (6 mL), treated with trifluoroacetic acid (1 mL), and stirred overnight at room temperature. The reaction mixture was purified by HPLC. Fractions that contained The title compound were combined and neutralized with aqueous NaHCO₃ and extracted with EtOAc. The organic layer was treated with 4 M HCl in 1,4-dioxane (0.2 mL) and concentrated in vacuo to afford the title compound as a yellow solid (211 mg).

¹H NMR (500 MHz, DMSO-d₆): δ 1.98-2.02 (m, 2H), 3.12-3.18 (m, 4H), 3.34 (t, J=6.0 Hz, 2H), 3.55 (m, 2H), 7.11 (d, J=16.6 Hz, 1H), 7.35 (d, J=16.5 Hz, 1H), 7.45 (d, J=8.4 Hz, 1H), 7.56 (dd, J=8.4, 1.3 Hz, 1H), 7.64 (dd, J=8.5, 2.0 Hz, 1H), 7.68 (dd, J=10.5, 2.0 Hz, 1H), 7.99 (s, 1H), 8.32 (t, J=8.2 Hz, 1H), 8.80 (s, 2H), 8.85-9.05 (br s, 1H), 9.49 (br s, 2H), 9.75 (s, 1H). ¹⁹F NMR (470 MHz, DMSO-d₆): δ −120.23 (s, 1F). MS (ES+): m/z 526 (M+H)⁺.

### Example 68

### tert-Butyl 4-(4-bromo-3-methylphenylsulfonyl)-1,4-diazepane-1-carboxylate (Intermediate 38)

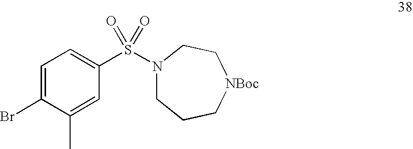

Triethylamine (2.1 mL, 15.0 mmol) was added to a solution of 1-Boc-hexahydro-1,4-diazepine (1.00 g, 5.0 mmol) and 4-bromo-3-methylbenzenesulfonyl chloride (1.35 g, 5.0 mmol) dissolved in DCM (20 mL). After stirring overnight at room temperature, the solution was diluted with EtOAc (40 mL), washed two times with saturated aqueous NaHCO₃ (2×15 mL), and washed with brine. The solution was dried over MgSO₄ and concentrated in vacuo to afford the title intermediate as pale yellow oil (2.06 g, 95%).

¹H NMR (500 MHz, DMSO-d₆): δ 1.37 (s, 9H), 1.66-1.76 (m, 2H), 2.43 (s, 3H), 3.17-3.22 (m, 2H), 3.27 (t, J=5.4 Hz, 2H), 3.29-3.35 (m, 2H), 3.39-3.43 (m, 2H), 7.51 (d, J=8.2 Hz, 1H), 7.78 (s, 1H), 7.81 (d, J=8.4 Hz, 1H). MS (ES+): m/z 455 (M+Na)⁺.

### Example 69

### tert-Butyl 4-(3-methyl-4-(5-vinylpyrimidin-2-ylamino)phenylsulfonyl)-1,4-diazepane-1-carboxylate (Intermediate 39)

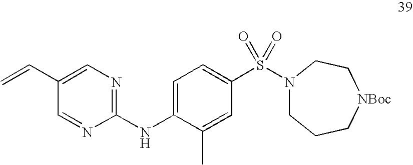

A suspension of intermediate 38 (1.07 g, 2.47 mmol), 1 (0.35 g, 2.47 mmol), Pd(OAc)₂ (28 mg, 0.12 mmol), Xantphos (0.14 g, 0.25 mmol), and potassium tert-butoxide (0.61 g, 5.44 mmol) in 1,4-dioxane (12 mL) and DMF (3 mL) was purged 5 min with argon gas. The suspension was sealed in a microwave reaction tube and irradiated with microwave 30 min at 180° C. After cooling to room temperature, the cap was removed and the resulting mixture was filtered through a pad of silica gel and the filtered solid was washed with EtOAc. The filtrate was concentrated and the residue was purified by silica gel chromatography (hexanes/EtOAc 10:0 to 6:4 gradient) to afford the title intermediate as orange oil (0.38 g, 17%).

¹H NMR (500 MHz, DMSO-d₆): δ 1.38 (s, 9H), 1.67-1.77 (m, 2H), 2.34 (s, 3H), 3.15-3.21 (m, 2H), 3.22-3.29 (m, 2H), 3.30-3.36 (m, 2H), 3.40-3.44 (m, 2H), 5.24 (d, J=11.6 Hz, 1H), 5.86 (d, J=17.8 Hz, 1H), 6.31 (dd, J=17.9, 11.2 Hz, 1H), 7.57 (d, J=8.5 Hz, 1H), 7.61 (s, 1H), 7.94 (d, J=8.4 Hz, 1H), 8.62 (s, 2H), 9.12 (s, 1H). MS (ES+): m/z 496 (M+Na)⁺.

### Example 70

### (E)-6-(2-(2-(4-(1,4-Diazepan-1-ylsulfonyl)-2-methylphenylamino)pyrimidin-5-yl)vinyl)benzo[d]thiazol-2-amine 2,2,2-trifluoroacetate (Compound XXX)

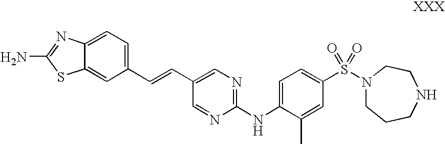

To a suspension of intermediate 39 (0.40 g, 0.85 mmol), 2-amino-6-bromobenzothiazole (0.39 g, 1.69 mmol), Pd₂(dba)₃ (77 mg, 0.08 mmol) and cesium carbonate (0.55 g, 1.69 mmol) in 1,4-dioxane (12 mL) was added a solution of 1 M tri-tert-butylphosphine in toluene (0.34 mL, 0.34 mmol). The suspension was purged 5 min with argon, sealed in a microwave reaction tube and irradiated with microwave 60 min at 180° C. After cooling to room temperature, the cap was removed and the resulting mixture was filtered through a pad of silica gel and the filtered solid was washed with EtOAc. The filtrate was concentrated and the residue was purified by silica gel chromatography (hexanes/EtOAc 10:0 to 3:7 gradient) to give a yellow solid. The yellow solid was dissolved in DCM (4 mL), treated with trifluoroacetic acid (0.5 mL), and stirred overnight at room temperature. The reaction mixture was purified by HPLC to afford the title compound as a yellow solid (104 mg).

¹H NMR (500 MHz, DMSO-d₆): δ 1.93-1.99 (m, 2H), 2.37 (s, 3H), 3.04-3.15 (m, 4H), 3.30 (t, J=6.1 Hz, 2H), 3.47 (t, J=6.1 Hz, 2H), 7.06 (d, J=16.5 Hz, 1H), 7.29 (d, J=16.6 Hz, 1H), 7.35 (d, J=8.3 Hz, 1H), 7.47 (d, J=7.1 Hz, 1H), 7.61 (d, J=8.7 Hz, 1H), 7.65 (s, 1H), 7.91 (s, 1H), 7.95-8.10 (br s, 2H), 8.04 (d, J=8.6 Hz, 1H), 8.70-8.76 (br s, 2H), 8.73 (s, 2H), 9.16 (s, 1H). MS (ES+): m/z 522 (M+H)⁺.

### Example 71

### 3-Bromophenylsulfamic acid (Intermediate 40)

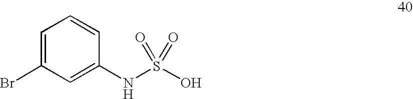

To a solution of 3-bromoaniline (1.00 g, 5.81 mmol) in DCM (15 mL) was added chlorosulfonic acid (0.39 mL, 5.81 mmol). The reaction mixture was stirred 20 at room temperature. The resulting precipitate was isolated by vacuum filtration to afford the title intermediate as a white solid (1.46 g, 100%). ¹H NMR (500 MHz, DMSO-d₆): δ 7.02 (d, J=7.7 Hz, 1H), 7.12-7.70 (br s, 2H), 7.21 (d, J=7.5 Hz, 1H), 7.22 (s, 1H), 7.25 (dd, J=7.8, 7.5 Hz, 1H).

### Example 72

### 2-Amino-4-bromobenzene-1-sulfonyl chloride (Intermediate 41)

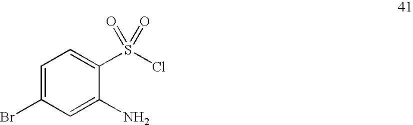

A solution of intermediate 40 (0.50 g, 1.98 mmol) in POCl₃ (10 mL) was heated 2 h under reflux. After cooling to room temperature, the reaction mixture was concentrated in vacuo and poured onto ice. The resulting precipitate was isolated by vacuum filtration to afford the title intermediate as a tan solid (0.31 g, 57%). ¹H NMR (500 MHz, DMSO-d₆): δ 6.96 (dd, J=8.3, 2.0 Hz, 1H), 7.06 (d, J=2.0 Hz, 1H), 7.46 (d, J=8.3 Hz, 1H).

### Example 73

### tert-Butyl 4-(2,4-diaminophenylsulfonyl)piperazine-1-carboxylate (Intermediate 42)

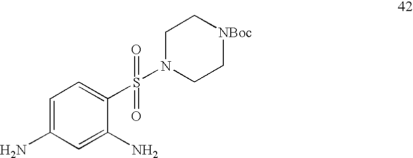

Triethylamine (0.77 mL, 5.55 mmol) was added to a solution of tert-butyl piperazine-1-carboxylate (0.34 g, 1.85 mmol) and intermediate 41 (0.50 g, 1.85 mmol) dissolved in DCM (10 mL). After stirring overnight at room temperature, the solution was diluted with EtOAc (20 mL), washed two times with saturated aqueous NaHCO₃ (2×10 mL), and washed with brine. The solution was dried over MgSO₄ and purified by silica gel chromatography (hexanes/EtOAc 10:0 to 6:4 gradient) to afford the title intermediate as a tan solid (0.22 g, 29%).

¹H NMR (500 MHz, DMSO-d₆): δ 1.35 (s, 9H), 2.94 (t, J=5.0 Hz, 4H), 3.35 (m, 4H), 6.26 (s, 2H), 6.79 (dd, J=8.6, 2.0 Hz, 1H), 7.09 (d, J=2.0 Hz, 1H), 7.29 (d, J=8.6 Hz, 1H). MS (ES+): m/z 322 (M-Boc+H)⁺.

### Example 74

### 4-[2-Amino-4-(5-vinyl-pyrimidin-2-ylamino)-benzenesulfonyl]-piperazine-1-carboxylic acid tert-butyl ester (Intermediate 43)

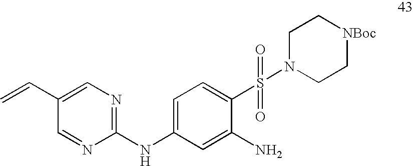

A suspension of intermediate 42 (0.10 g, 0.24 mmol), intermediate 1 (35 mg, 0.29 mmol), Pd₂(dba)₃ (22 mg, 0.024 mmol), Xantphos (28 mg, 0.048 mmol), and cesium carbonate (0.16 g, 0.48 mmol) in 1,4-dioxane (3 mL) and DMF (1 mL) was purged 5 min with argon gas. The suspension was sealed in a microwave reaction tube and irradiated with microwave 30 min at 160° C. After cooling to room temperature, the cap was removed and the resulting mixture was filtered through a pad of silica gel and the filtered solid was washed with EtOAc. The filtrate was concentrated and the residue was purified by silica gel chromatography (hexanes/EtOAc 10:0 to 5:5 gradient) to afford the title intermediate as orange oil (87 mg, 79%). MS (ES+): m/z 461 (M+H)⁺.

### Example 75

### (E)-N¹-(5-(2-(2-Aminobenzo[d]thiazol-6-yl)vinyl)pyrimidin-2-yl)-4-(piperazin-1-ylsulfonyl)benzene-1,3-diamine hydrochloride (Compound XXXI)

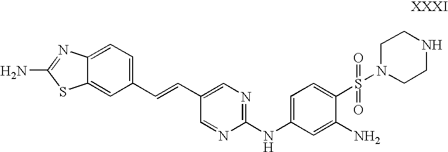

To a suspension of intermediate 43 (85 mg, 0.18 mmol), 2-amino-6-bromobenzothiazole (85 mg, 0.37 mmol), Pd₂(dba)₃ (17 mg, 0.02 mmol) and cesium carbonate (0.12 g, 0.37 mmol) in 1,4-dioxane (3 mL) was added a solution of 1 M tri-tert-butylphosphine in toluene (0.074 mL, 0.074 mmol). The suspension was purged 5 min with argon, sealed in a microwave reaction tube and irradiated with microwave 30 min at 180° C. After cooling to room temperature, the cap was removed and the resulting mixture was filtered through a pad of silica gel and the filtered solid was washed with EtOAc. The filtrate was concentrated and the residue was purified by silica gel chromatography (hexanes/EtOAc 10:0 to 3:7 gradient) to give a yellow solid. The yellow solid was dissolved in DCM (3 mL), treated with trifluoroacetic acid (0.5 mL), and stirred overnight at room temperature. The reaction mixture was purified by HPLC. Fractions that contained The title compound were combined and neutralized with aqueous NaHCO₃ and extracted with EtOAc. The organic layer was treated with 4 M HCl in 1,4-dioxane (0.2 mL) and concentrated in vacuo to afford the title compound as a yellow solid (86 mg).

¹H NMR (500 MHz, DMSO-d₆): δ 3.08 (s, 4H), 3.24 (m, 4H), 2.90-3.30 (br s, 2H), 7.03 (dd, J=9.0, 2.0 Hz, 1H), 7.15 (d, J=16.5 Hz, 1H), 7.31 (d, J=9.0 Hz, 1H), 7.35 (d, J=16.5 Hz, 1H), 7.51 (d, J=2.0 Hz, 1H), 7.52 (d, J=8.5 Hz, 1H), 7.61 (d, J=7.3 Hz, 1H), 8.05 (s, 1H), 8.79 (s, 2H), 9.68 (s, 4H), 10.08 (s, 1H). MS (ES+): m/z 509 (M+H)⁺.

### Example 76

### {5-[2-(3-Nitro-phenyl)-vinyl]-pyrimidin-2-yl}-pyridin-3-yl-amine (Intermediate 44)

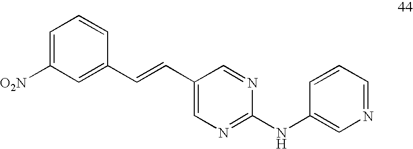

A suspension of intermediate 7 (0.24 g, 0.90 mmol), 3-bromo-pyridine (0.10 mL, 1.0 mmol), Pd₂(dba)₃ (50 mg, 0.055 mmol), Xantphos (65 mg, 0.11 mmol) and cesium carbonate (0.70 g, 2.1 mmol) in dioxane/DMF (6/1, 7 mL) was sealed in a microwave reaction tube and irradiated with microwave at 160° C. for 20 min. After cooling to room temperature, the cap was removed and the resulting mixture filtered and the filtered solid washed with DCM. The filtrate was concentrated and the residue purified by flash chromatography on silica gel (hexanes to EtOAc) to afford the title intermediate as a yellow solid (0.17 g, 59%). MS (ES+): m/z 320 (M+H)⁺.

### Example 77

### {5-[2-(3-Amino-phenyl)-vinyl]-pyrimidin-2-yl}-pyridin-3-yl-amine (Intermediate 45)

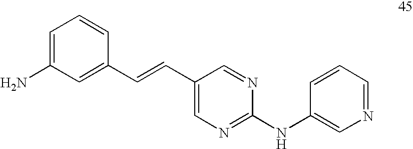

A mixture of intermediate 44 (0.17 g, 0.53 mmol) and sodium sulfide (0.50 g, 2.1 mmol) in ethanol (20 mL) was heated at reflux for 1.5 h. After cooling to RT, the mixture was poured into water. The aqueous layer was extracted with EtOAc and the organic layer separated. The organic extract was washed with brine, dried over anhydrous Na₂SO₄ and then filtered. The filtrate was concentrated and used in the next step without purification. MS (ES+): m/z 290 (M+H)⁺.

### Example 78

### N-(3-{2-[2-(Pyridin-3-ylamino)-pyrimidin-5-yl]-vinyl}-phenyl)-3-trifluoromethyl-benzamide (Compound XXXII)

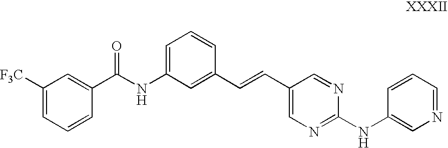

To a mixture of intermediate 45 (0.15 g, 0.50 mmol) and 3-trifluoromethyl-benzoyl chloride (0.15 mL, 1.0 mmol) in DCM (15 mL) was added triethylamine (0.20 mL, 1.4 mmol). The resulting mixture was stirred at RT for 18 h and then poured into water (30 mL). The aqueous layer was extracted with EtOAc (50 mL) and the organic layer separated. The organic extract was washed with brine, dried over anhydrous Na₂SO₄ and filtered. The filtrate was concentrated and the residue purified by HPLC. The fractions were combined and poured into saturated NaHCO₃ solution (30 mL). The combined aqueous layers were extracted with EtOAc (2×30 mL) and the combined organic layers washed with brine, dried over anhydrous Na₂SO₄ and filtered. The filtrate was concentrated to afford the title compound as a white solid (5 mg, 2%).

¹H NMR (500 MHz, DMSO-d₆): δ 7.12 (d, J=16.6 Hz, 1H), 7.32 (d, J=16.7 Hz, 1H), 7.30-7.42 (m, 3H), 7.67 (d, J=8.0 Hz, 1H), 7.81 (t, J=7.8 Hz, 1H), 7.99 (d, J=7.7 Hz, 1H), 8.02 (s, 1H), 8.17 (dd, J=4.6, 1.4 Hz, 1H), 8.22-8.25 (m, 1H), 8.30 (d, J=7.9 Hz, 1H), 8.33 (s, 1H), 8.83 (s, 2H), 8.91 (d, J=2.6 Hz, 1H), 10.01 (s, 1H), 10.54 (s, 1H). MS (ES+): m/z 462 (M+H)⁺.

### Example 79

### (5-Bromo-pyridin-2-yl)-[4-(2-hydroxy-ethyl)-piperazin-1-yl]-methanone (Intermediate 46)

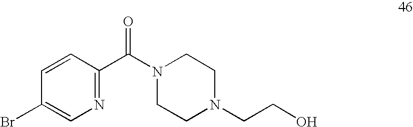

To a solution of 5-bromo-pyridine-2-carboxylic acid (1.0 g, 5.0 mmol) and 2-piperazin-1-yl-ethanol (1.0 g, 7.7 mmol) in dry DMF (20 mL) was added HBTU (2.8 g, 7.4 mmol) and HOBt (0.88 g, 6.5 mmol) followed by DIPEA (2.6 mL, 15 mmol). The reaction mixture was stirred at room temperature for 16 h and then diluted with EtOAc. The reaction mixture was washed with water, brine, dried over anhydrous MgSO₄ and filtered. The filtrate was concentrated in vacuo and The residue triturated in hexanes/Et₂O (5:1 v/v) and the white solid filtered (1.0 g, 65%). MS (ES+): m/z 314 (M+H)⁺.

### Example 80

### [4-(2-Hydroxy-ethyl)-piperazin-1-yl]-(5-{5-[2-(1H-indol-4-yl)-vinyl]-pyrimidin-2-ylamino}-pyridin-2-yl)-methanone (Compound XXXIII)

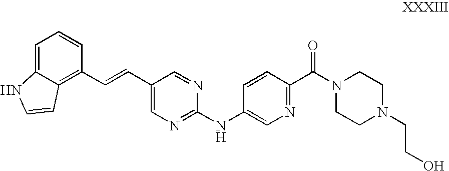

A suspension of intermediate 11 (0.10 g, 0.42 mmol), intermediate 46 (0.20 g, 0.64 mmol), Pd₂(dba)₃ (25 mg, 0.027 mmol), Xantphos (30 mg, 0.052 mmol) and cesium carbonate (0.30 g, 0.92 mmol) in dioxane/DMF (3/1, 4 mL) was sealed in a microwave reaction tube and irradiated with microwave at 160° C. for 20 min. After cooling to room temperature, the cap was removed and the resulting mixture filtered and the filtered solid washed with DCM. The filtrate was concentrated and the residue purified by HPLC. The fractions were combined and poured into saturated NaHCO₃ solution (30 mL). The combined aqueous layers were extracted with EtOAc (2×30 mL) and the combined organic layers washed with brine, dried over anhydrous Na₂SO₄ and filtered. The filtrate was concentrated and the residue triturated in EtOAc/hexanes (1/8, 45 mL). After filtration, The title compound was obtained as a white solid (15 mg, 8%).

¹H NMR (500 MHz, DMSO-d₆): δ 2.42 (t, J=6.2 Hz, 4H), 3.51 (q, J=6.0 Hz, 2H), 3.53-3.60 (m, 2H), 3.60-3.70 (m, 2H), 4.43 (t, J=5.3 Hz, 1H), 6.94 (t, J=1.0 Hz, 1H), 7.11 (t, J=7.7 Hz, 1H), 7.25 (d, J=16.7 Hz, 1H), 7.32 (d, J=7.2 Hz, 1H), 7.35 (d, J=8.1 Hz, 1H), 7.43 (t, J=2.8 Hz, 1H), 7.60 (d, J=8.6 Hz, 1H), 7.67 (d, J=16.7 Hz, 1H), 8.37 (dd, J=8.6, 2.6 Hz, 1H), 8.92 (d, J=3.0 Hz, 1H), 8.93 (s, 2H), 10.23 (s, 1H), 11.22 (s, 1H). MS (ES+): m/z 470 (M+H)⁺.

### Example 81

### (5-Bromo-indazol-1-yl)-phenyl-methanone (Intermediate 47)

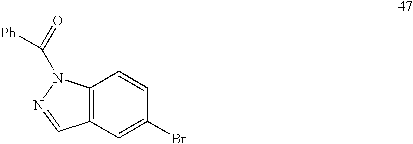

To a solution of 5-bromo-1H-indazole (0.60 g, 3.1 mmol) in dry DCM (15 mL) was added benzoyl chloride (0.40 mL, 3.4 mmol) followed by triethylamine (0.60 mL, 4.3 mmol). The reaction mixture was stirred at room temperature for 16 h and then poured into saturated NaHCO₃ solution (50 mL). The mixture was extracted with EtOAc (2×50 mL) and the combined extracts washed with brine, dried over anhydrous Na₂SO₄ and filtered. The filtrate was concentrated in vacuo and the residue triturated in methanol and the white solid filtered (0.73 g, 80%). MS (ES+): m/z 300 (M+H)⁺.

### Example 82

### {5-[2-(2-Amino-pyrimidin-5-yl)-vinyl]-indazol-1-yl}-phenyl-methanone (Intermediate 48)

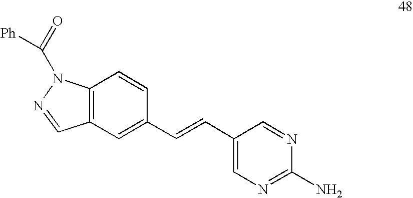

A mixture of intermediate 47 (0.10 g, 0.33 mmol), intermediate 1 (45 mg, 0.37 mmol), Pd(OAc)₂ (5 mg, 0.022 mmol), and triethylamine (0.60 mL, 4.3 mmol) in DMF (15 mL) was heated at 110° C. under the argon atmosphere for 16 h. After cooling to RT, the mixture was poured into water. The aqueous layer was extracted with EtOAc and the organic layer separated. The organic extract was washed with brine, dried over anhydrous Na₂SO₄ and then filtered. The filtrate was concentrated and the residue purified by flash chromatography on silica gel (DCM to 10% MeOH/DCM) to afford the title intermediate as a brown solid (0.50 g, 73%). MS (ES+): m/z 342 (M+H)⁺.

### Example 83

### {5-[2-(1H-Indazol-5-yl)-vinyl]-pyrimidin-2-yl}-[4-(Piperidine-4-sulfonyl)-phenyl]-amine (Compound XXXIV)

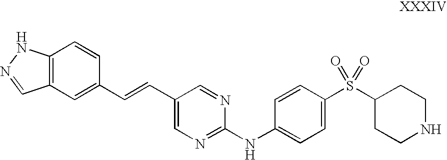

A suspension of intermediate 48 (0.50 g, 1.5 mmol), intermediate 5 (0.75 g, 1.9 mmol), Pd₂(dba)₃ (80 mg, 0.087 mmol), Xantphos (0.10 g, 0.17 mmol) and cesium carbonate (1.0 g, 3.1 mmol) in dioxane (25 mL) was heated at reflux under the argon atmosphere for 15 h. After cooling to room temperature, the mixture was filtered and the filtered solid washed with DCM. The filtrate was concentrated and the residue purified HPLC. The fractions were combined and poured into saturated NaHCO₃ solution (50 mL). The combined aqueous layers were extracted with EtOAc (2×50 mL) and the combined organic layers washed with brine, dried over anhydrous Na₂SO₄ and filtered. The filtrate was concentrated and then re-dissolved in DCM (5 mL). TFA (3 mL) was added and the mixture stirred at RT for 15 min. The reaction was concentrated and purified by HPLC again. The fractions were combined and poured into saturated NaHCO₃ solution (30 mL). The combined aqueous layers were extracted with EtOAc (2×30 mL) and the combined organic extracts washed with brine, dried over anhydrous Na₂SO₄ and filtered. The filtrate was concentrated and the resulting solid dissolved in minimum amount of EtOAc and hexanes added until solid precipitated. After filtration, The title compound was obtained as a white solid (3 mg, 1% in 2 steps).

¹H NMR (500 MHz, DMSO-d₆): δ 1.30-1.42 (m, 2H), 1.70-1.80 (m, 2H), 2.95-3.05 (m, 2H), 3.15-3.25 (m, 1H), 7.14 (d, J=16.6 Hz, 1H), 7.46 (d, J=16.7 Hz, 1H), 7.56 (d, J=8.8 Hz, 1H), 7.69 (dd, J=8.8, 1.4 Hz, 1H), 7.73 (d, J=8.9 Hz, 2H), 7.88 (s, 1H), 8.05 (d, J=8.9 Hz, 2H), 8.10 (s, 1H), 8.85 (s, 2H), 10.37 (s, 1H), 13.14 (br s, 1H). MS (ES+): m/z 461 (M+H)⁺.

### Example 84

### [1-(4-Bromo-benzenesulfonyl)-piperidin-4-yl]-methanol (Intermediate 49)

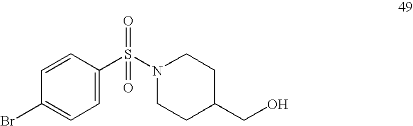

To a solution of 4-bromo-benzenesulfonyl chloride (4.0 g, 16 mmol) and piperidin-4-yl-methanol (2.0 g, 17 mmol) in dry DCM (35 mL) was added triethylamine (3.0 mL, 22 mmol). The reaction mixture was stirred at room temperature for 21 h and then diluted with EtOAc (50 mL). The combined organic layer was washed with saturated NaHCO₃, brine, dried over anhydrous Na₂SO₄ and filtered. The filtrate was concentrated and the residue triturated in hexanes. After filtration. The title intermediate was obtained as an off white solid (4.8 g, 92%). MS (ES+): m/z 334 (M+H)⁺.

### Example 85

### {1-[4-(5-Vinyl-pyrimidin-2-ylamino)-benzenesulfonyl]-piperidin-4-yl}-methanol (Intermediate 50)

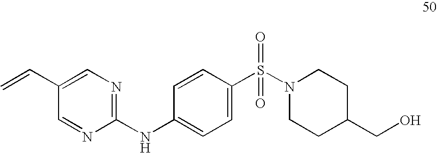

A suspension of intermediate 1 (0.20 g, 1.7 mmol), intermediate 49 (0.60 mL, 1.8 mmol), Pd₂(dba)₃ (0.10 g, 0.11 mmol), Xantphos (0.13 g, 0.22 mmol) and cesium carbonate (1.1 g, 3.4 mmol) in dioxane/DMF (3/1, 8 mL) was sealed in a microwave reaction tube and irradiated with microwave at 160° C. for 20 min. After cooling to room temperature, the cap was removed and the resulting mixture filtered and the filtered solid washed with DCM. The filtrate was concentrated and then re-dissolved in minimum amount of MeOH. Ether was added until solid precipitated and the solid filtered. The filtrate was concentrated to afford the title intermediate (0.20 g, 31%), which was used in the next step without purification. MS (ES+): m/z 375 (M+H)⁺.

### Example 86

### [1-(4-{5-[2-(2-Amino-benzothiazol-6-yl)-vinyl]-pyrimidin-2-ylamino}-benzenesulfonyl)-piperidin-4-yl]-methanol (Compound XXXV)

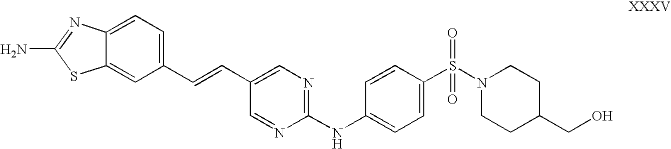

A suspension of intermediate 50 (0.20 g, 0.53 mmol), 6-bromo-benzothiazol-2-ylamine (0.13 g, 0.56 mmol), Pd(OAc)₂ (10 mg, 0.044 mmol), PPh₃ (17 mg, 0.065 mmol) and triethylamine (0.15 mL, 1.1 mmol) in DMF (3 mL) was sealed in a microwave reaction tube and irradiated with microwave at 170° C. for 15 min. After cooling down to RT, the cap was removed and the resulting mixture filtered and the filtered solid washed with MeOH. The filtrate was concentrated and the residue purified by HPLC. The combined fractions were poured into saturated NaHCO₃ solution (30 mL) and then extracted with EtOAc (2×30 mL). The combined organic extracts were washed with brine, dried over anhydrous Na₂SO₄ and filtered. The filtrate was concentrated and the residue triturated in MeOH. After filtration, The title compound was obtained as a light brown solid (15 mg, 6%).

¹H NMR (500 MHz, DMSO-d₆): δ 1.08-1.20 (m, 2H), 1.25-1.35 (m, 1H), 1.70 (d, J=10.5 Hz, 2H), 2.17 (t, J=10.9 Hz, 2H), 3.20 (t, J=5.7 Hz, 2H), 3.61 (d, J=11.8 Hz, 2H), 4.47 (t, J=5.2 Hz, 1H), 7.07 (d, J=16.6 Hz, 1H), 7.31 (d, J=8.9 Hz, 1H), 7.34 (s, 1H), 7.44 (dd, J=8.4, 1.5 Hz, 1H), 7.58 (s, 1H), 7.65 (d, J=9.1 Hz, 2H), 7.88 (d, J=1.3 Hz, 1H), 8.02 (d, J=8.9 Hz, 2H), 8.80 (s, 2H), 10.31 (s, 1H). MS (ES+): m/z 523 (M+H)⁺.

### Example 87

### 4-[4-(5-Formyl-pyrimidin-2-ylamino)-benzenesulfonyl]-piperazine-1-carboxylic acid tert-butyl ester (Intermediate 51)

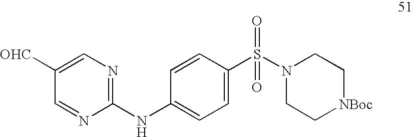

A suspension of 2-amino-pyrimidine-5-carbaldehyde (0.25 g, 2.0 mmol), intermediate 24 (0.91 g, 2.3 mmol), Pd₂(dba)₃ (0.12 g, 0.13 mmol), Xantphos (0.15 g, 0.26 mmol) and cesium carbonate (1.3 g, 4.0 mmol) in dioxane/DMF (3/1, 8 mL) was sealed in a microwave reaction tube and irradiated with microwave at 160° C. for 20 min. After cooling to room temperature, the cap was removed and the resulting mixture filtered and the filtered solid washed with DCM. The filtrate was concentrated and the residue triturated in MeOH. After filtration, The title intermediate was obtained as a light brown solid (0.62 g, 68%). MS (ES+): m/z 448 (M+H)⁺.

### Example 88

### 4-(4-{5-[2-(4-Bromo-phenyl)-vinyl]-pyrimidin-2-ylamino}-benzenesulfonyl)-piperazine-1-carboxylic acid tert-butyl ester (Intermediate 52)

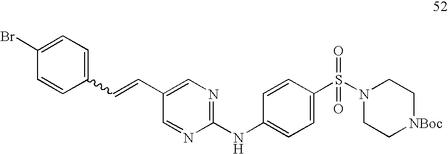

To a suspension of (4-bromo-benzyl)-triphenyl-phosphonium bromide (0.20 g, 0.45 mmol) in THF (15 mL) under the argon atmosphere was added n-butyl lithium (2.5 M in hexanes; 0.40 mL, 1.0 mmol) at RT. After stirring at RT for 10 min, intermediate 51 (0.20 g, 0.45 mmol) was added and the resulting mixture stirred at RT for additional 2 h. The reaction was quenched with water and the mixture extracted with EtOAc (30 mL). The extract was washed with brine, dried over Na₂SO₄ and filtered. The filtrate was concentrated and the residue triturated in MeOH. After filtration, The title intermediate in a mixture of cis/trans isomer was obtained as a brown solid (0.12 g, 46%). MS (ES+): m/z 600 (M+H)⁺.

### Example 89

### [4-(piperazine-1-sulfonyl)-phenyl]-(5-{2-[4-(1H-pyrazol-4-yl)-phenyl]-vinyl}-pyrimidin-2-yl)-amine (Compound XXXVI)

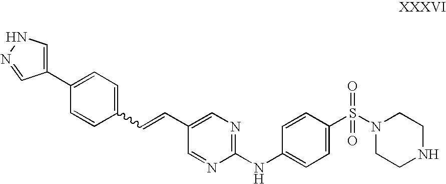

A suspension of intermediate 52 (0.10 g, 0.16 mmol), 1H-indol-4-yl-4-boronic acid (80 g, 0.41 mmol), Pd(PPh₃)₄ (20 mg, 0.018 mmol) and 2M of Na₂CO₃ solution (0.2 mL, 0.4 mmol) in DMF (6 mL) was sealed in a microwave reaction tube and irradiated with microwave at 170° C. for 20 min. After cooling down to RT, the cap was removed and the resulting mixture filtered and the filtered solid washed with DCM. The filtrate was concentrated and the residue purified by column chromatography (hexanes to 80% EtOAc/hexanes) to obtain the protected precursor. To the precursor in DCM (4 mL), TFA (2 mL) was added and the mixture stirred at RT for 2 h. The reaction mixture was concentrated and the residue purified by HPLC. The corrected fractions were combined and poured into saturated NaHCO₃ solution (30 mL). The aqueous layer was extracted with EtOAc (2×30 mL) and the combined organic extracts washed with brine, dried over anhydrous Na₂SO₄ and filtered. The filtrate was concentrated and the residue triturated in EtOAc/hexanes (1/5, 30 mL). After filtration, The title compound in a mixture of cis/trans isomer was obtained as a white solid (19 mg, 24%).

¹H NMR of cis isomer (500 MHz, DMSO-d₆): δ 2.65-2.80 (m, 8H), 6.49 (d, J=12.1 Hz, 1H), 6.72 (d, J=12.1 Hz, 1H), 7.27 (d, J=8.3 Hz, 2H), 7.50-7.60 (m, 4H), 7.97 (d, J=8.9 Hz, 2H), 8.43 (s, 2H), 10.28 (s, 1H), 12.97 (br s, 1H). ¹H NMR of trans isomer (500 MHz, DMSO-d₆): δ 2.65-2.80 (m, 8H), 7.15 (d, J=16.7 Hz, 1H), 7.33 (d, J=16.7 Hz, 1H), 7.50-7.60 (m, 4H), 7.64 (d, J=8.6 Hz, 2H), 8.03 (d, J=8.8 Hz, 2H), 8.84 (s, 2H), 10.35 (s, 1H), 12.97 (br s, 1H). MS of the mixture (ES+): m/z 488 (M+H)⁺.

### Example 90

### 3-(3-Bromo-phenyl)-propan-1-ol (Intermediate 53)

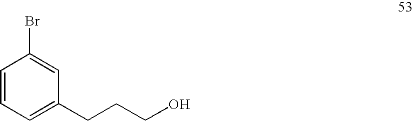

3-(3-Bromo-phenyl)-propionic acid (3.9 g, 17 mmol, 1 equiv) was diluted with THF (50 mL) and chilled to 0° C. 1M of LAH solution (3 equiv) was added slowly to the above solution so as to not allow internal reaction temperature to climb above 10-15° C. Once LAH addition was complete, reaction was allowed to come to room temperature and stir for additional 3 h. Reaction was quenched with sequential addition of water (0.5 mL), 15% NaOH (0.5 mL) and water again (1.5 mL). This was then filtered and solvents evaporated to provide the intermediate as pale oil (2.75 g, 98%). R_{f}=0.42 (30% EtOAc/hexanes).

### Example 91

### 1-Bromo-3-(3-bromo-propyl)-benzene (Intermediate 54)

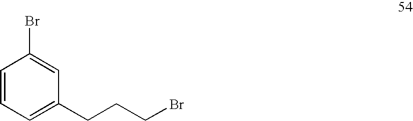

To a solution of intermediate 53 (4.0 g, 19 mmol, 1 equiv) in THF (100 mL) was added CBr₄ (9.3 g, 28 mmol, 1.5 equiv) and triphenyl phosphine (7.3 g, 28 mmol, 1.5 equiv). The resulting mixture was stirred at RT for 16 h and then diluted with EtOAc (125 mL). The combined organic layer was washed with water, brine, dried over anhydrous Na₂SO₄ and filtered. The filtrate was concentrated to provide The title intermediate as clear oil (4.0 g, 78%).

### Example 92

### 1-[3-(3-Bromo-phenyl)-propyl]-pyrrolidine (Intermediate 55)

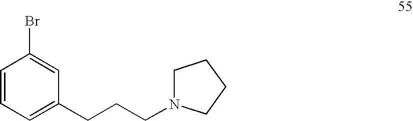

Bromide intermediate 54 (1.0 g, 3.7 mmol, 1 equiv) was diluted with dioxane (30 mL), treated with pyrrolidine (0.61 mL, 7.35 mmol, 2 equiv), cesium carbonate (2.4 g, 7.35 mmol, 2 equiv) and stirred at RT for 18 h. Reaction was then diluted with water (125 mL) and extracted with EtOAc (2×100 mL). Organic phase was cut from aqueous phase, dried over sodium sulfate, filtered and the filtrate evaporated to provide the title intermediate as clear oil (0.6 g, 61%).

### Example 93

### 6-[2-(2-Amino-pyrimidin-5-yl)-vinyl]-benzothiazol-2-ylamine (Intermediate 56)

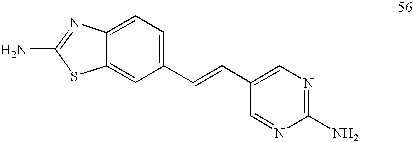

A suspension o intermediate 1 (0.20 g, 1.7 mmol), 6-bromo-benzothiazol-2-ylamine (0.43 g, 1.9 mmol), Pd(OAc)₂ (25 mg, 0.11 mmol), PPh₃ (40 mg, 0.15 mmol) and triethylamine (0.40 mL, 2.9 mmol) in DMF (3 mL) was sealed in a microwave reaction tube and irradiated with microwave at 170° C. for 15 min. After cooling down to RT, the cap was removed and the resulting mixture filtered and the filtered solid washed with MeOH. The filtrate was concentrated and the residue triturated in MeOH. After filtration, The title intermediate was obtained as a light brown solid (0.10 g, 22%). (ES+): m/z 270 (M+H)⁺.

### Example 94

### 6-(2-{2-[4-(3-Pyrrolidin-1-yl-propyl)-phenylamino]-pyrimidin-5-yl}-vinyl)-benzothiazol-2-ylamine (Compound XXXVII)

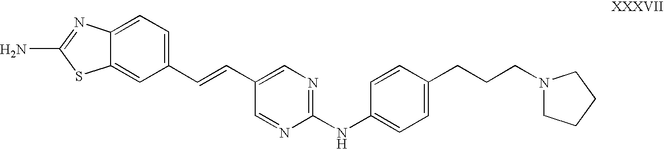

A suspension of intermediate 56 (0.10 g, 0.37 mmol), intermediate 55 (0.12 g, 0.45 mmol), Pd₂(dba)₃ (20 mg, 0.022 mmol), Xantphos (25 mg, 0.044 mmol) and cesium carbonate (0.25 g, 0.77 mmol) in dioxane/DMF (3/1, 4 mL) was sealed in a microwave reaction tube and irradiated with microwave at 180° C. for 20 min. After cooling to room temperature, the cap was removed and the resulting mixture filtered and the filtered solid washed with DCM. The filtrate was concentrated and the residue purified by HPLC. The combined fractions were poured into saturated NaHCO₃ solution (30 mL) and then extracted with EtOAc (2×30 mL). The combined organic extracts were washed with brine, dried over anhydrous Na₂SO₄ and filtered. The filtrate was concentrated and the residue triturated in EtOAc/hexanes (1/2, 30 mL). After filtration, The title compound was obtained as a light brown solid (33 mg, 20%).

¹H NMR (500 MHz, DMSO-d₆): δ 1.75-1.90 (m, 6H), 2.60 (t, J=7.5 Hz, 2H), 2.70-2.95 (m, 6H), 6.54 (d, J=16.5 Hz, 1H), 6.60 (s, 1H), 6.83 (d, J=16.5 Hz, 1H), 6.89 (d, J=8.3 Hz, 1H), 7.03 (s, 1H), 7.11 (d, J=8.2 Hz, 2H), 7.16 (d, J=8.3 Hz, 2H), 7.22 (dd, J=8.4, 2.0 Hz, 1H), 8.36 (s, 2H). MS (ES+): m/z 457 (M+H)⁺.

### Example 95

### [1-(4-{5-[2-(2-Amino-indan-5-yl)-vinyl]-pyrimidin-2-ylamino}-benzenesulfonyl)-piperidin-4-yl]-methanol (Compound XXXVIII)

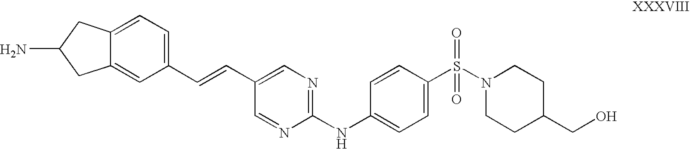

A suspension of intermediate 50 (0.20 g, 0.53 mmol), 5-bromo-indan-2-ylamine (0.20 g, 0.68 mmol), Pd(OAc)₂ (10 mg, 0.044 mmol), PPh₃ (20 mg, 0.076 mmol) and triethylamine (0.20 mL, 1.4 mmol) in DMF (3 mL) was sealed in a microwave reaction tube and irradiated with microwave at 170° C. for 20 min. After cooling down to RT, the cap was removed and the resulting mixture filtered and the filtered solid washed with DCM. The filtrate was concentrated and the residue purified by HPLC. The corrected fractions were combined and poured into saturated NaHCO₃ solution (30 mL). The aqueous layer was extracted with EtOAc (2×30 mL) and the combined organic extracts washed with brine, dried over anhydrous Na₂SO₄ and filtered. The filtrate was concentrated and the solid dissolved in minimum amount of EtOAc and hexanes added until solid precipitated. After filtration, The title compound was obtained as a brown solid (16 mg, 6%).

¹H NMR (500 MHz, DMSO-d₆): δ 1.10-1.45 (m, 3H), 1.69 (d, J=11.9 Hz, 2H), 2.16 (t, J=11.2 Hz, 2H), 2.50-2.65 (m, 2H), 3.00-3.10 (m, 2H), 3.19 (t, J=5.4 Hz, 2H), 3.61 (d, J=11.7 Hz, 2H), 3.71 (t, J=6.3 Hz, 1H), 4.52 (t, J=5.1 Hz, 1H), 7.11 (d, J=16.6 Hz, 1H), 7.19 (d, J=7.8 Hz, 1H), 7.30 (d, J=16.7 Hz, 1H), 7.31 (d, J=7.6 Hz, 1H), 7.42 (s, 1H), 7.64 (d, J=8.9 Hz, 2H), 8.03 (d, J=9.0 Hz, 2H), 8.81 (s, 2H), 10.33 (s, 1H). MS (ES+): m/z 506 (M+H)⁺.

### Example 97

### [1-(4-{5-[2-(3-Methoxy-phenyl)-vinyl]-pyrimidin-2-ylamino}-benzenesulfonyl)-piperidin-4-yl]-methanol (Intermediate 57)

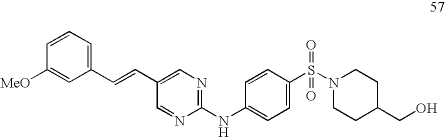

A suspension of intermediate 50 (0.25 g, 0.67 mmol), 1-bromo-3-methoxy-benzene (0.10 mL, 0.80 mmol), Pd(OAc)₂ (10 mg, 0.044 mmol), PPh₃ (20 mg, 0.076 mmol) and triethylamine (0.20 mL, 1.4 mmol) in DMF (4 mL) was sealed in a microwave reaction tube and irradiated with microwave at 180° C. for 20 min. After cooling down to RT, the cap was removed and the resulting mixture filtered and the filtered solid washed with DCM. The filtrate was concentrated and the residue purified by flash chromatography on silica gel (hexanes to 70% EtOAc/hexanes) to afford the title intermediate as a brown oil (0.14 g, 43%). MS (ES+): m/z 481 (M+H)⁺.

### Example 98

### 3-(2-{2-[4-(4-Hydroxymethyl-piperidine-1-sulfonyl)-phenylamino]-pyrimidin-5-yl}-vinyl)-phenol (Compound XXXIX)

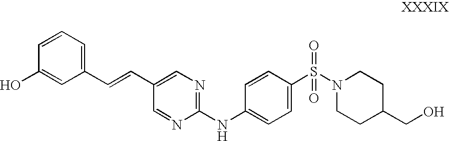

To a suspension of intermediate 57 (0.14 g, 0.29 mmol) in DCM (10 mL) at 0° C. was added BBr₃ (1M in DCM; 1.5 mL, 1.5 mmol) and the mixture stirred at room temperature for 1 h. The reaction was quenched with saturated NaHCO₃ solution until the pH ˜7. The resulting solution was extracted with EtOAc (30 mL) and the extract washed with brine, dried over anhydrous Na₂SO₄ and filtered. The filtrate was concentrated and the solid dissolved in minimum amount of EtOAc and hexanes added until solid precipitated. After filtration, The title compound was obtained as a white solid (15 mg, 11%).

¹H NMR (500 MHz, DMSO-d₆): δ 1.10-1.35 (m, 3H), 1.70 (d, J=10.6 Hz, 2H), 2.17 (t, J=10.8 Hz, 2H), 3.20 (t, J=5.7 Hz, 2H), 3.61 (d, J=11.6 Hz, 2H), 3.47 (t, J=5.2 Hz, 1H), 6.70 (dd, J=7.9, 2.4 Hz, 1H), 6.96 (t, J=1.9 Hz, 1H), 7.01 (d, J=7.9 Hz, 1H), 7.08 (d, J=16.7 Hz, 1H), 7.18 (t, J=7.9 Hz, 1H), 7.25 (d, J=16.6 Hz, 1H), 7.65 (d, J=9.0 Hz, 2H), 8.02 (d, J=8.9 Hz, 2H), 8.83 (s, 2H), 9.47 (s, 1H), 10.33 (s, 1H). MS (ES+): m/z 467 (M+H)⁺.

### Example 99

### 4-(4-Vinyl-phenyl)-1H-imidazole (Intermediate 58)

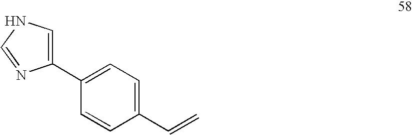

A suspension of 4-iodo-1H-imidazole (0.50 g, 2.6 mmol), 4-vinyl-phenyl-boronic acid (0.40 g, 2.7 mmol), Pd(PPh₃)₄ (0.25 g, 0.22 mmol) and 2M of Na₂CO₃ solution (3.0 mL, 6.0 mmol) in DMF (5 mL) was sealed in a microwave reaction tube and irradiated with microwave at 160° C. for 20 min. After cooling down to RT, the cap was removed and the resulting mixture diluted with MeOH (5 mL). The resulting solid was filtered and the filtrate poured into water. The mixture was extracted with EtOAc and the organic layer washed with brine, dried over anhydrous Na₂SO₄ and filtered. The filtrate was concentrated and the residue purified by column chromatography on silica gel (hexanes to 70% EtOAc/hexanes) to obtain The title intermediate as a colorless glassy substance (80 mg, 18%).

### Example 100

### 4-[4-(5-Bromo-pyrimidin-2-ylamino)-benzenesulfonyl]-piperazine-1-carboxylic acid tert-butyl ester (Intermediate 59)

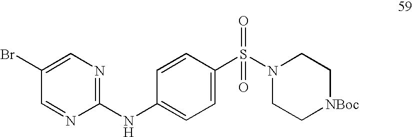

A suspension of 5-bromo-pyrimidin-2-ylamine (0.30 g, 1.7 mmol), intermediate 24 (1.0 g, 2.5 mmol), Pd₂(dba)₃ (0.15 g, 0.16 mmol), Xantphos (0.15 g, 0.26 mmol) and cesium carbonate (1.1 g, 3.4 mmol) in dioxane/DMF (3/1, 8 mL) was sealed in a microwave reaction tube and irradiated with microwave at 160° C. for 20 min. After cooling to room temperature, the cap was removed and the resulting mixture filtered and the filtered solid washed with DCM. The filtrate was concentrated and the solid dissolved in minimum amount of EtOAc and hexanes added until solid precipitated. After filtration, The title intermediate was obtained as a yellow solid (0.70 g, 81%). MS (ES+): m/z 498/500 (M+H)⁺.

### Example 101

### (5-{2-[4-(1H-Imidazol-4-yl)-phenyl]-vinyl}-pyrimidin-2-yl)-[4-(piperazine-1-sulfonyl)-phenyl]-amine (Compound XL)

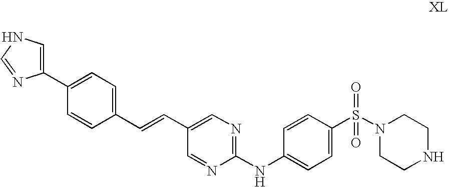

A suspension of intermediate 58 (80 mg, 0.47 mmol), intermediate 59 (0.20 g, 0.40 mmol), Pd(OAc)₂ (10 mg, 0.044 mmol), PPh₃ (20 mg, 0.076 mmol) and triethylamine (0.50 mL, 3.6 mmol) in DMF (4 mL) was sealed in a microwave reaction tube and irradiated with microwave at 170° C. for 20 min. After cooling down to RT, the cap was removed and the resulting mixture filtered and the filtered solid washed with DCM. The filtrate was concentrated and the protected precursor re-dissolved in DCM (5 mL). TFA (3 mL) was added to the above solution and the mixture stirred at RT for 1.5 h. The reaction mixture was concentrated and the residue purified by HPLC. The corrected fractions were combined and poured into saturated NaHCO₃ solution (30 mL). The aqueous layer was extracted with EtOAc (2×30 mL) and the combined organic extracts washed with brine, dried over anhydrous Na₂SO₄ and filtered. The filtrate was concentrated, the residue re-dissolved in minimum amount of EtOAc and hexanes added until solid precipitated. After filtration, The title compound was obtained as a yellow solid (5 mg, 2% in 2 steps).

¹H NMR (500 MHz, DMSO-d₆): δ 2.65-2.80 (m, 8H), 7.14 (d, J=16.5 Hz, 1H), 7.33 (d, J=16.6 Hz, 1H), 7.56 (d, J=8.1 Hz, 2H), 7.55-7.70 (m, 5H), 7.81 (d, J=7.7 Hz, 2H), 8.03 (d, J=8.9 Hz, 2H), 8.84 (s, 2H), 10.34 (s, 1H), 12.19 (br s, 1H). MS (ES+): m/z 488 (M+H)⁺.

### Example 102

### {5-[2-(1H-Indazol-4-yl)-vinyl]-pyrimidin-2-yl}-[4-(piperazine-1-sulfonyl)-phenyl]-amine (Compound XLI)

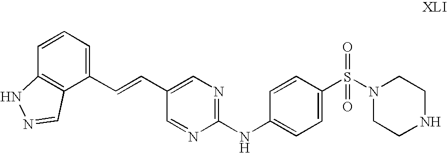

A suspension of intermediate 25 (0.10 g, 0.22 mmol), 4-bromo-1H-indazole (60 mg, 0.31 mmol), Pd(OAc)₂ (6 mg, 0.027 mmol), PPh₃ (14 mg, 0.053 mmol) and triethylamine (0.15 mL, 1.1 mmol) in DMF (3 mL) was sealed in a microwave reaction tube and irradiated with microwave at 180° C. for 15 min. After cooling down to RT, the cap was removed and the resulting mixture filtered and the filtered solid washed with DCM. The filtrate was concentrated and the residue purified by flash chromatography on silica gel (hexanes to 70% EtOAc/hexanes) to afford the protected precursor. To a suspension of the precursor in DCM (5 mL) was added TFA (2 mL) and the mixture stirred at RT for 30 min. The reaction mixture was concentrated and the residue purified by HPLC. The corrected fractions were combined and poured into saturated NaHCO₃ solution (30 mL). The aqueous layer was extracted with EtOAc (2×30 mL) and the combined organic extracts washed with brine, dried over anhydrous Na₂SO₄ and filtered. The filtrate was concentrated, the residue re-dissolved in minimum amount of EtOAc and hexanes added until solid precipitated. After filtration, The title compound was obtained as a yellow solid (40 mg, 36% in 2 steps).

¹H NMR (500 MHz, DMSO-d₆): δ 2.65-2.80 (m, 8H), 7.34-7.40 (m, 2H), 7.42 (d, J=16.7 Hz, 1H), 7.45-7.50 (m, 1H), 7.65 (d, J=9.0 Hz, 2H), 7.71 (d, J=16.8 Hz, 1H), 8.06 (d, J=8.9 Hz, 2H), 8.60 (s, 1H), 8.98 (s, 2H), 10.40 (s, 1H), 13.30 (br s, 1H),

MS (ES+): m/z 462 (M+H)⁺.

### Example 103

### N-[6-(2-{2-[4-(piperazine-1-sulfonyl)-phenylamino]-pyrimidin-5-yl}-vinyl)-benzothiazol-2-yl]-3-trifluoromethyl-benzamide (Compound XLII)

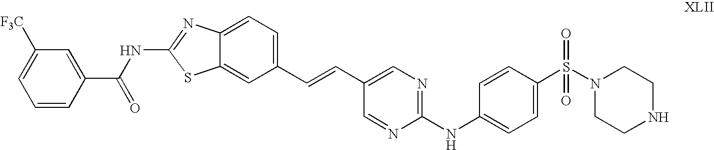

A suspension of intermediate 16 (0.10 g, 0.25 mmol), intermediate 25 (0.12 g, 0.27 mmol), Pd(OAc)₂ (6 mg, 0.027 mmol), PPh₃ (12 mg, 0.046 mmol) and triethylamine (0.30 mL, 2.2 mmol) in DMF (3 mL) was sealed in a microwave reaction tube and irradiated with microwave at 180° C. for 20 min. After cooling down to RT, the cap was removed and the resulting mixture filtered and the filtered solid washed with DCM. The filtrate was concentrated and the residue purified by flash chromatography on silica gel (hexanes to 60% EtOAc/hexanes) to afford the protected precursor. To a suspension of the precursor in DCM (5 mL) was added TFA (2 mL) and the mixture stirred at RT for 1 h. The reaction mixture was concentrated and the residue purified by HPLC. The corrected fractions were combined and poured into saturated NaHCO₃ solution (30 mL). The aqueous layer was extracted with EtOAc (2×30 mL) and the combined organic extracts washed with brine, dried over anhydrous Na₂SO₄ and filtered. The filtrate was concentrated, the residue re-dissolved in minimum amount of EtOAc and hexanes added until solid precipitated. After filtration, The title compound was obtained as a yellow solid (10 mg, 6% in 2 steps).

¹H NMR (500 MHz, DMSO-d₆): δ 2.70-2.80 (m, 8H), 7.22 (d, J=16.6 Hz, 1H), 7.44 (d, J=16.6 Hz, 1H), 7.60-7.70 (m, 3H), 7.73 (d, J=8.4 Hz, 1H), 7.80 (t, J=7.9 Hz, 1H), 7.99 (d, J=7.7 Hz, 1H), 8.06 (d, J=8.9 Hz, 2H), 8.16 (s, 1H), 8.42 (d, J=7.9 Hz, 1H), 8.53 (s, 1H), 8.86 (s, 2H), 10.37 (s, 1H). MS (ES+): m/z 666 (M+H)⁺.

### Example 104

### 4-[4-(5-Vinyl-pyrimidin-2-ylamino)-benzenesulfonylamino]-piperidine-1-carboxylic acid tert-butyl ester (Intermediate 60)

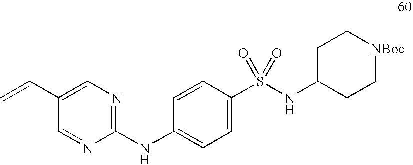

A suspension of intermediate 12 (0.50 g, 1.2 mmol), intermediate 1 (0.17 g, 1.4 mmol), Pd₂(dba)₃ (0.10 g, 0.11 mmol), Xantphos (0.13 g, 0.22 mmol) and cesium carbonate (0.80 g, 2.4 mmol) in dioxane (20 mL) was heated at reflux under the argon atmosphere for 3 h. After cooling to room temperature, the mixture was filtered and the filtered solid washed with DCM. The filtrate was concentrated and the residue purified by flash chromatography on silica gel (hexanes to 50% EtOAc/hexanes) to afford the title intermediate as an off white solid (0.40 g, 73%). MS (ES+): m/z 460 (M+H)⁺.

### Example 105

### 4-{5-[2-(2-Amino-benzothiazol-6-yl)-vinyl]-pyrimidin-2-ylamino}-N-piperidin-4-yl-benzenesulfonamide (Compound XLIII)

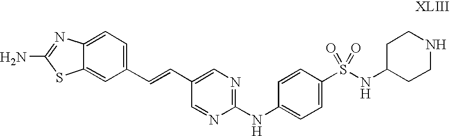

A suspension of intermediate 60 (0.10 g, 0.22 mmol), 6-bromo-benzothiazol-2-ylamine (60 mg, 0.26 mmol), Pd(OAc)₂ (5 mg, 0.025 mmol), PPh₃ (10 mg, 0.038 mmol) and triethylamine (0.15 mL, 1.1 mmol) in DMF (3 mL) was sealed in a microwave reaction tube and irradiated with microwave at 180° C. for 20 min. After cooling down to RT, the cap was removed and the resulting mixture filtered and the filtered solid washed with DCM. The filtrate was concentrated and the residue purified by flash chromatography on silica gel (hexanes to EtOAc) to afford the protected precursor. To a suspension of the precursor in DCM (10 mL) was added TFA (2 mL) and the mixture stirred at RT for 1 h. The reaction mixture was concentrated and the residue purified by HPLC. The corrected fractions were combined and poured into saturated NaHCO₃ solution (30 mL). The aqueous layer was extracted with EtOAc (2×30 mL) and the combined organic extracts washed with brine, dried over anhydrous Na₂SO₄ and filtered. The filtrate was concentrated and the residue triturated in EtOAc/hexanes (1/5, 30 mL). After filtration, The title compound was obtained as a yellow solid (20 mg, 18% in 2 steps).

¹H NMR (500 MHz, DMSO-d₆): δ 1.20-1.30 (m, 2H), 1.50-1.60 (m, 2H), 2.41 (t, J=10.9 Hz, 2H), 2.86 (d, J=12.6 Hz, 2H), 2.95-3.05 (m, 1H), 7.07 (d, J=16.5 Hz, 1H), 7.31 (d, J=16.5 Hz, 1H), 7.33 (d, J=8.3 Hz, 1H), 7.44 (dd, J=8.5, 1.6 Hz, 1H), 7.50-7.60 (m, 3H), 7.72 (d, J=8.9 Hz, 2H), 7.88 (d, J=1.5 Hz, 1H), 7.96 (d, J=8.9 Hz, 2H), 8.79 (s, 2H), 10.23 (s, 1H). MS (ES+): m/z 508 (M+H)⁺.

### Example 106

### 3-(2-{2-[4-([1,4]Diazepane-1-sulfonyl)-phenylamino]-pyrimidin-5-yl}-vinyl)-benzenesulfonamide (Compound XLIV)

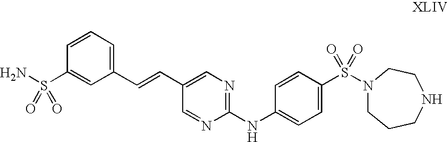

A suspension of intermediate 31 (0.10 g, 0.22 mmol), 3-bromo-benzenesulfonamide (60 mg, 0.25 mmol), Pd(OAc)₂ (5 mg, 0.025 mmol), PPh₃ (10 mg, 0.038 mmol) and triethylamine (0.15 mL, 1.1 mmol) in DMF (3 mL) was sealed in a microwave reaction tube and irradiated with microwave at 180° C. for 20 min. After cooling down to RT, the cap was removed and the resulting mixture filtered and the filtered solid washed with DCM. The filtrate was concentrated and the residue purified by flash chromatography on silica gel (hexanes to 90% EtOAc/hexanes) to afford the protected precursor. To a suspension of the precursor in DCM (10 mL) was added TFA (3 mL) and the mixture stirred at RT for 1 h. The reaction mixture was concentrated and the residue purified by HPLC. The corrected fractions were combined and poured into saturated NaHCO₃ solution (30 mL). The aqueous layer was extracted with EtOAc (2×30 mL) and the combined organic extracts washed with brine, dried over anhydrous Na₂SO₄ and filtered. The filtrate was concentrated and the residue triturated in EtOAc/hexanes (1/5, 30 mL). After filtration, The title compound was obtained as a white solid (20 mg, 18% in 2 steps).

¹H NMR (500 MHz, DMSO-d₆): δ 1.60-1.67 (m, 2H), 2.69 (t, J=5.9 Hz, 2H), 2.70-2.75 (m, 2H), 3.15-3.18 (m, 2H), 3.25 (t, J=6.0 Hz, 2H), 7.28 (d, J=16.6 Hz, 1H), 7.40 (br s, 2H), 7.44 (d, J=16.6 Hz, 1H), 7.59 (t, J=7.8 Hz, 1H), 7.71 (d, J=8.9 Hz, 2H), 7.79 (d, J=7.8 Hz, 1H), 7.88 (d, J=7.8 Hz, 1H), 8.00 (d, J=8.8 Hz, 2H), 8.02 (s, 1H), 8.15 (t, J=1.7 Hz, 1H), 8.88 (s, 2H), 10.34 (s, 1H). MS (ES+): m/z 515 (M+H)⁺.

### Example 107

### 4-(2-{2-[4-([1,4]Diazepane-1-sulfonyl)-phenylamino]-Pyrimidin-5-yl}-vinyl)-benzenesulfonamide (Compound XLV)

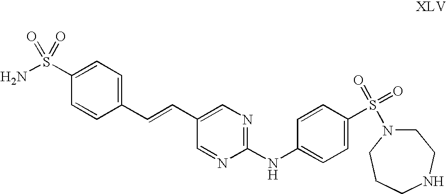

A suspension of intermediate 31 (0.10 g, 0.22 mmol), 4-bromo-benzenesulfonamide (60 mg, 0.25 mmol), Pd(OAc)₂ (5 mg, 0.025 mmol), PPh₃ (10 mg, 0.038 mmol) and triethylamine (0.15 mL, 1.1 mmol) in DMF (3 mL) was sealed in a microwave reaction tube and irradiated with microwave at 180° C. for 20 min. After cooling down to RT, the cap was removed and the resulting mixture filtered and the filtered solid washed with DCM. The filtrate was concentrated and the residue purified by flash chromatography on silica gel (hexanes to 90% EtOAc/hexanes) to afford the protected precursor. To a suspension of the precursor in DCM (10 mL) was added TFA (3 mL) and the mixture stirred at RT for 1 h. The reaction mixture was concentrated and the residue purified by HPLC. The corrected fractions were combined and poured into saturated NaHCO₃ solution (30 mL). The aqueous layer was extracted with EtOAc (2×30 mL) and the combined organic extracts washed with brine, dried over anhydrous Na₂SO₄ and filtered. The filtrate was concentrated and the residue triturated in EtOAc/hexanes (1/5, 30 mL). After filtration, The title compound was obtained as an off white solid (35 mg, 31% in 2 steps).

¹H NMR (500 MHz, DMSO-d₆): δ 1.63-1.73 (m, 2H), 2.70-2.85 (m, 4H), 3.15-3.25 (m, 2H), 3.26 (t, J=5.9 Hz, 2H), 7.34 (d, J=16.7 Hz, 1H), 7.40 (d, J=16.8 Hz, 1H), 7.71 (d, J=8.8 Hz, 2H), 7.74 (d, J=8.5 Hz, 2H), 7.82 (d, J=8.4 Hz, 2H), 7.93 (s, 2H), 8.00 (d, J=8.8 Hz, 2H), 8.87 (s, 2H), 10.36 (s, 1H). MS (ES+): m/z 515 (M+H)⁺.

### Example 108

### 1-(4-Bromo-benzenesulfonyl)-4-(2-methoxy-ethyl)-piperazine (Intermediate 61)

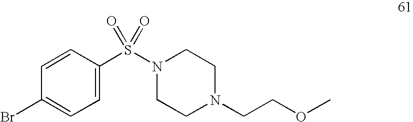

To a solution of 4-bromo-benzenesulfonyl chloride (1.6 g, 6.3 mmol) and 1-(2-methoxy-ethyl)-piperazine (1.0 g, 6.9 mmol) in dry DCM (35 mL) was added triethylamine (1.5 mL, 11 mmol). The reaction mixture was stirred at room temperature for 15 h and then poured into saturated NaHCO₃ solution. The mixture was extracted with EtOAc and the organic layer separated. The organic extract was washed with brine, dried over Na₂SO₄ and filtered. The filtrate was concentrated, the residue re-dissolved in minimum amount of EtOAc and hexanes added until solid precipitated. After filtration, The title intermediate was obtained as a white solid (2.1 g, 92%). MS (ES+): m/z 363 (M+H)⁺.

### Example 109

### {4-[4-(2-Methoxy-ethyl)-piperazine-1-sulfonyl]-phenyl}-(5-vinyl-pyrimidin-2-yl)-amine (Intermediate 62)

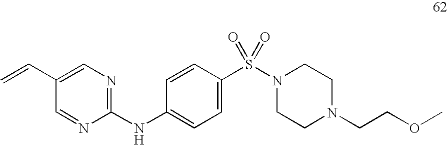

A suspension of intermediate 61 (1.0 g, 2.8 mmol), intermediate 1 (0.30 g, 2.5 mmol), Pd₂(dba)₃ (0.20 g, 0.22 mmol), Xantphos (0.25 g, 0.43 mmol) and cesium carbonate (1.7 g, 5.2 mmol) in dioxane (30 mL) was heated at reflux under the argon atmosphere for 3 h. After cooling to room temperature, the mixture was filtered and the filtered solid washed with DCM. The filtrate was concentrated and the residue purified by flash chromatography on silica gel (hexanes to EtOAc) to afford the title intermediate as a brown gel (0.43 g, 43%). MS (ES+): m/z 404 (M+H)⁺.

### Example 110

### 6-[2-(2-{4-[4-(2-Methoxy-ethyl)-piperazine-1-sulfonyl]-phenylamino}-pyrimidin-5-yl)-vinyl]-benzothiazol-2-ylamine (Compound XLVI)

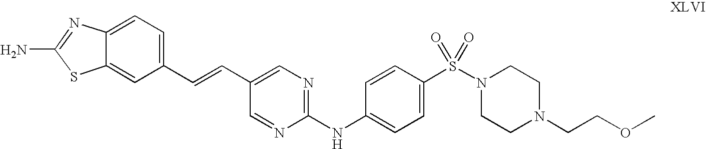

A suspension of intermediate 62 (0.15 g, 0.37 mmol), 6-bromo-benzothiazol-2-ylamine (85 mg, 0.37 mmol), Pd(OAc)₂ (10 mg, 0.044 mmol), PPh₃ (20 mg, 0.076 mmol) and triethylamine (0.30 mL, 2.2 mmol) in DMF (3 mL) was sealed in a microwave reaction tube and irradiated with microwave at 180° C. for 20 min. After cooling down to RT, the cap was removed and the resulting mixture filtered and the filtered solid washed with MeOH. The filtrate was concentrated and the residue purified by HPLC. The corrected fractions were combined and poured into saturated NaHCO₃ solution (30 mL). The aqueous layer was extracted with EtOAc (2×30 mL) and the combined organic extracts washed with brine, dried over anhydrous Na₂SO₄ and filtered. The filtrate was concentrated and the residue triturated in EtOAc/hexanes (1/5, 30 mL). After filtration, The title compound was obtained as a yellow solid (35 mg, 17%).

¹H NMR (500 MHz, DMSO-d₆): δ 2.40-2.50 (m, 6H), 2.80-2.90 (m, 4H), 3.17 (s, 3H), 3.35 (d, J=5.7 Hz, 2H), 7.07 (d, J=16.6 Hz, 1H), 7.32 (d, J=16.3 Hz, 1H), 7.33 (d, J=8.4 Hz, 1H), 7.44 (dd, J=8.4, 1.7 Hz, 1H), 7.58 (s, 1H), 7.65 (d, J=8.9 Hz, 2H), 7.88 (d, J=1.7 Hz, 1H), 8.04 (d, J=8.9 Hz, 2H), 8.81 (s, 2H), 10.33 (s, 1H). MS (ES+): m/z 552 (M+H)⁺.

### Example 111

### 4-(5-((E)-2-(1H-indol-4-yl)vinyl)pyrimidin-2-ylamino)benzonitrile (Compound XLVII)

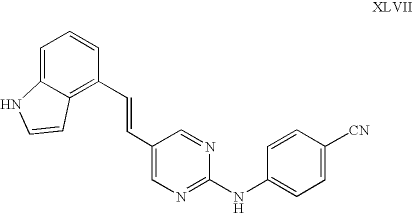

A mixture of 5-((E)-2-(1H-indol-4-yl)vinyl)pyrimidin-2-amine (236 mg, 1.0 mmol), 4-bromobenzonitrile (182 mg, 0.46 mmol), Pd₂(dba)₃ (18 mg, 0.02 mmol), xantphos (23 mg, 0.04 mmol) and cesium carbonate (975 mg, 3.0 mmol) in dioxane (5 mL) was heated under refluxed for 16 h under argon. The reaction mixture was cooled to room temperature, the dioaxne was removed using rotoevaporator, and the residue was triturated with DMF (5 mL), and the solid removed through centrifugation. The supernatant on purification by HPLC gave The title compound as yellow solid (272 mg, 80%).

¹H NMR (500 MHz, DMSO-d₆): δ 6.93-6.97 (m, 1H), 7.11 (d, J=7.65 Hz, 1H), 6.26 (d, J=16.7 Hz, 1H), 7.30-7.37 (m, 2H), 7.43-7.47 (m, 1H), 7.68 (d, J=16.8, 1H), 7.74 (d, J=9.1 Hz, 2H), 8.00 (d, J=9.1 Hz, 2H), 8.94 (s, 2H), 10.35 (s, 1H), 11.22 (br s, 1H). MS (ES+): m/z 338 (M+H)⁺.

### Example 112

### 5-((E)-2-(1H-benzo[d]imidazol-5-yl)vinyl)-N-(4-(piperidin-4-ylsulfonyl)phenyl)pyrimidin-2-amine (Compound XLVIII)

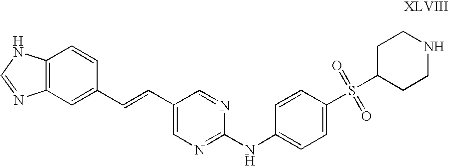

A mixture of (5-bromo-1H-benzo[d]imidazol-1-yl)(phenyl)methanone (60 mg, 0.20 mmol), intermediate 60 (88 mg, 0.20 mmol), Pd(OAc)₂ (0.89 mg, 0.033 mmol), triphenyl phosphine (4.2 mg, 0.016 mmol) and KHCO₃ (40 mg, 0.4 mmol) in DMF (2 mL) was sealed in a microwave reaction vial and irradiated with microwave at 160° C. for 30 min. The reaction mixture was cooled to room temperature and purified by HPLC, and treated with TFA (2 mL) in dichloromethane (10 mL) for 10 minutes. The solvent was evaporated and the residue was purified by HPLC to give the title compound as cream colored solid (6 mg, 6%).

¹H NMR (500 MHz, DMSO-d₆): δ 1.62-1.72 (m, 2H), 2.01-2.07 (m, 2H), 2.50-2.62 (m, 4H overlapped with DMSO), 2.75-2.85 (m, 2H), 7.23 (d, J=17 Hz, 1H), 7.51 (d, J=16.5 Hz, 1H), 7.72 (s, 1H), 7.76 (d, J=8.5 Hz, 1H), 7.85 (s, 1H), 7.95 (s, 1H), 8.08 (d, J=8.8 Hz, 2H), 8.88 (s, 2H), 10.46 (s, 1H). MS (ES+): m/z 461 (M+H)⁺.

### Example 113

### 7-((E)-2-(2-(4-(piperidin-4-ylsulfonyl)phenylamino)pyrimidin-5-yl)vinyl)-5-(trifluoromethyl)-1H-benzo[d]imidazol-2-amine (Compound XLIX)

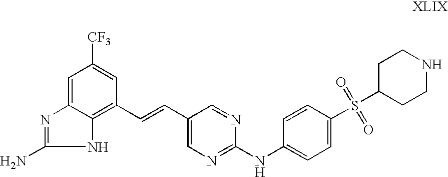

A mixture of 3-bromo-4,5-diamino-benzotrifluoride (255 mg, 1.0 mmol), intermediate 60 (444 mg, 1.0 mmol), Pd(OAc)₂ (4.5 mg, 0.02 mmol), triphenyl phosphine (21 mg, 0.08 mmol) and KHCO₃ (200 mg, 2.0 mmol) in DMF (5 mL) was sealed in a microwave reaction vial and irradiated with microwave at 180° C. for 30 min. The reaction mixture was purified by HPLC to give a 3-((E)-2-(2-(4-(piperidin-4-ylsulfonyl)phenylamino)pyrimidin-5-yl)vinyl)-5-(trifluoromethyl)benzene-1,2-diamine intermediate (183 mg, 30%). This diamino intermediate was dissolved in methanol (10 mL) and diluted with water (10 mL). To this clear solution was added cyanogen bromide (100 μl, 3M DCM solution, 0.32 mmol) and stirring continued for 16 h at room temperature. The solvent evaporated and the residue was purified by HPLC, after treating with TFA (1 mL) to give the title compound as cream colored solid (12 mg, 46% based on starting material recovered).

¹H NMR (500 MHz, DMSO-d₆): δ 1.62-1.75 (m, 2H), 2.01-2.07 (m, 2H), 2.85-2.95 (m, 2H), 3.33-3.43 (m, 2H), 7.51 (s, 1H), 7.58 (d, J=16.6 Hz, 1H), 7.64 (d, J=16.0 Hz, 1H), 7.77 (d, J=9.0 Hz, 2H), 7.84 (s, 1H), 8.09 (d, J=8.9 Hz, 2H), 8.27-8.29 (m, 1H), 8.61-8.67 (m, 2H), 8.88 (s, 2H), 10.56 (s, 1H). MS (ES+): m/z 544 (M+H)⁺.

### Example 114

### 5-((E)-2-(6-(trifluoromethyl)-1H-benzo[d][1,2,3]triazol-4-yl)vinyl)-N-(4-(Piperidin-4-ylsulfonyl)phenyl)pyrimidin-2-amine (Compound L)

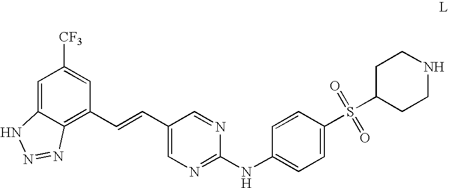

A mixture of 3-bromo-4,5-diamino-benzotrifluoride (255 mg, 1.0 mmol), intermediate 60 (444 mg, 1.0 mmol), Pd(OAc)₂ (4.5 mg, 0.02 mmol), triphenyl phosphine (21 mg, 0.08 mmol) and KHCO₃ (200 mg, 2.0 mmol) in DMF (5 mL) was sealed in a microwave reaction vial and irradiated with microwave at 180° C. for 30 min. The reaction mixture was purified by HPLC to give an intermediate, 3-((E)-2-(2-(4-(piperidin-4-ylsulfonyl)phenylamino)pyrimidin-5-yl)vinyl)-5-(trifluoromethyl)benzene-1,2-diamine (183 mg, 30%). This diamino intermediate (22 mg, 0.035 mmol) was suspended in water (1.0 mL) and conc. HCl (100 μl) was added. The mixture was treated with aqueous sodium nitrite solution (138 mg in 500 μL of water, 2 mmol) in two portions about five minutes apart. The reaction mixture was stirred for another ten minutes at room temperature, when effervescence subsides. The solvent was evaporated to dryness, and the yellow solid was treated with TFA (1 mL) in DCM (10 mL) for ten minutes. TFA was neutralized with TEA and the volatiles evaporated. The residue was purified by HPLC to give the title compound as cream colored solid (13 mg, 76%).

¹H NMR (500 MHz, DMSO-d₆): δ 1.62-1.75 (m, 2H), 2.01-2.09 (m, 2H), 2.80-2.95 (m, 2H), 3.35-3.40 (m, 2H, superimposed with H₂O), 3.45-3.56 (m, 1H), 7.78 (d, J=9.0 Hz, 2H), 7.75-7.86 (m, 2H, superimposed), 8.10 (d, J=9.3 Hz, 2H), 8.15-8.27 and 8.55-8.67 (2m, 1H each), 8.97 (br s, 2H), 10.57 (s, 1H). MS (ES+): m/z 530 (M+H)⁺.

### Example 115

### 5-((E)-2-(6-(trifluoromethyl)-1H-benzo[d]imidazol-4-yl)vinyl)-N-(4-(piperidin-4-ylsulfonyl)phenyl)pyrimidin-2-amine (Compound LI)

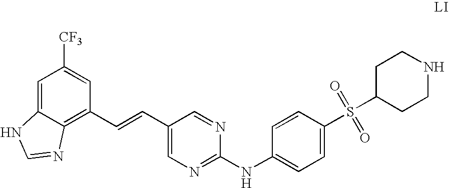

A mixture of 3-bromo-4,5-diamino-benzotrifluoride (255 mg, 1.0 mmol), intermediate 60 (444 mg, 1.0 mmol), Pd(OAc)₂ (4.5 mg, 0.02 mmol), triphenyl phosphine (21 mg, 0.08 mmol) and KHCO₃ (200 mg, 2.0 mmol) in DMF (5 mL) was sealed in a microwave reaction vial and irradiated with microwave at 180° C. for 30 min. The reaction mixture was purified by HPLC to give an intermediate, 3-((E)-2-(2-(4-(piperidin-4-ylsulfonyl)phenylamino)pyrimidin-5-yl)vinyl)-5-(trifluoromethyl)benzene-1,2-diamine (183 mg, 30%). This diamino intermediate (20 mg) was dissolved in formic acid (1.5 mL) heated under microwave at 150° C. for 30 minutes. The reaction mixture was purified by HPLC to give the title compound as cream colored solid (19 mg, quantitative).

¹H NMR (500 MHz, DMSO-d₆): δ 1.62-1.75 (m, 2H), 2.00-2.07 (m, 2H), 2.85-2.95 (m, 2H), 3.30-3.40 (m, 2H), 7.72-7.80 (m, 4H), 7.86 (s, 1H), 8.19 (d, J=8.5 Hz, 2H), 8.54-8.62 (m, 2H), 8.92 (s, 2H), 10.51 (s, 1H). MS (ES+): m/z 529 (M+H)⁺.

### Example 116

### 5-((E)-2-(1H-benzo[d][1,2,3]triazol-5-yl)vinyl)-N-(4-(piperidin-4-ylsulfonyl)phenyl)pyrimidin-2-amine (Compound LII)

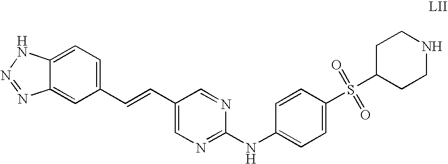

A mixture of 4-bromobenzene-1,2-diamine (187 mg, 1.0 mmol), intermediate 60 (444 mg, 1.0 mmol), Pd₂(dba)₃ (91 mg, 0.1 mmol), tert-tributyl phosphine (4 mL, 0.4 mmol, 1 M toluene solution) and Cs₂CO₃ (650 mg, 2.0 mmol) in dioxane (12 mL) was sealed in a microwave reaction vial and irradiated with microwave at 180° C. for 45 min. The reaction mixture was purified by HPLC to give an intermediate, 3-((E)-2-(2-(4-(piperidin-4-ylsulfonyl)phenylamino)pyrimidin-5-yl)vinyl)benzene-1,2-diamine (230 mg, 51%) as dark solid. This diamino intermediate (45 mg, 0.1 mmol) was suspended in water (5 mL) and conc. HCl (200 μl) was added cautiously. The mixture was treated with aqueous sodium nitrite solution (145 mg in 500 μL of water, 2.1 mmol) in two portions about five minutes apart. The reaction mixture was stirred for another 16 h at room temperature. The solvent evaporated to dryness, and the residue was treated with TFA (3 mL) in DCM (30 mL) for 10 minutes. The solvent evaporated and the residue purified by HPLC to give a white solid (5 mg, 11%).

¹H NMR (500 MHz, DMSO-d₆): δ 1.62-1.75 (m, 2H), 2.00-2.07 (m, 2H), 2.85-2.95 (m, 2H), 3.45-3.55 (m, 1H), 7.33 (d, J=16.6 Hz, 1H), 7.55 (d, J=16.5 Hz, 1H), 7.76 (d, J=8.8 Hz, 2H), 7.89-8.03 (m, 2H), 8.09 (d, J=8.8 Hz, 2H), 8.89 (s, 2H), 10.48 (s, 1H). MS (ES+): m/z 462 (M+H)⁺.

### Example 117

### [4-(piperazine-1-sulfonyl)-phenyl]-{5-[2-(6-trifluoromethyl-1H-benzotriazol-4-yl)-vinyl]-pyrimidin-2-yl}-amine (Compound LIII)

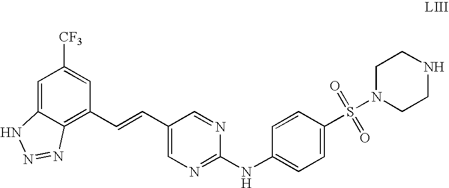

A mixture of 3-bromo-4,5-diamino-benzotrifluoride (138 mg, 0.54 mmol), intermediate 25 (200 mg, 0.45 mmol), Pd(OAc)₂ (4.5 mg, 0.02 mmol), triphenyl phosphine (21 mg, 0.08 mmol) and KHCO₃ (200 mg, 2.0 mmol) in DMF (5 mL) was sealed in a microwave reaction vial and irradiated with microwave at 180° C. for 30 min. The reaction mixture was purified by HPLC to give an intermediate, 3-((E)-2-(2-(4-(piperidin-4-ylsulfonyl)phenylamino)pyrimidin-5-yl)vinyl)-5-(trifluoromethyl)benzene-1,2-diamine (264 mg, 94%). This diamino intermediate (130 mg, 0.21 mmol) was suspended in water (2.5 mL) and conc. HCl (100 μl) added. The mixture was treated with aqueous sodium nitrite solution (145 mg in 500 μL of water, 2.1 mmol) in two portions about five minutes apart. The reaction mixture was stirred for another ten minutes at room temperature, when effervescence subsides. The solvent evaporated to dryness, and the residue was purified by HPLC to give a yellow solid. The solid was dissolved in methanol (3 mL) and treated with methanolic HCl (1.25 M, 10 mL) for 16 h. The solvent evaporated, the residue triturated with methanol-ethyl acetate-hexanes to give the title compound as yellow colored solid (11 mg, 11%).

¹H NMR (500 MHz, DMSO-d₆): δ 3.05-3.46 (m, 8H), 7.73 (d, J=8.7 Hz, 2H), 7.60-7.90 (m, 2H), 8.10 (d, J=8.7 Hz, 2H), 8.71 and 8.98 (br s, 2H each), 10.53 (s, 1H). MS (ES+): m/z 531 (M+H)⁺.

### Example 118

### [4-(piperazine-1-sulfonyl)-phenyl]-{5-[2-(6-chloro-1H-benzotriazol-4-yl)-vinyl]-pyrimidin-2-yl}-amine (Compound LIV)

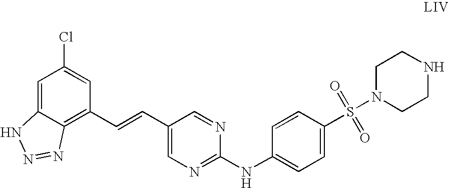

A mixture of 3-bromo-5-chlorobenzene-1,2-diamine (265 mg, 1.2 mmol), intermediate 25 (444 mg, 1.0 mmol), Pd₂(dba)₃ (91 mg, 0.1 mmol), tributyl phosphine (4 mL, 0.4 mmol, 1M toluene solution) and Cs₂CO₃ (650 mg, 2.0 mmol) in dioxane (12 mL) was sealed in a microwave reaction vial and microwave-irradiated at 180° C. for 30 min. The reaction mixture was purified by HPLC to give an intermediate, 3-((E)-2-(2-(4-(piperidin-4-yl sulfonyl)phenylamino)pyrimidin-5-yl)vinyl)-5-chloro-benzene-1,2-diamine (110 mg, 46% based on starting material recovered) as dark solid. This diamino intermediate (110 mg, 0.18 mmol) was suspended in water (5 mL) and conc. HCl (200 μl) was added. The mixture was treated with aqueous sodium nitrite solution (145 mg in 500 μL of water, 2.1 mmol) in two portions about five minutes apart. The reaction mixture was stirred for another 20 minutes at room temperature. The solvent evaporated to dryness, and the residue was purified by HPLC to give a yellow solid. The solid was suspended in methanol (5 mL) and treated with methanolic HCl (6 mL, 1.25 M solution) for 16 h at room temperature. The solvent was evaporated, and the sticky material was triturated with methanol-ethyl acetate hexanes to give a yellow solid (21 mg, 23%).

¹H NMR (500 MHz, DMSO-d₆): δ 3.05-3.27 (m, 8H), 2.00-2.07 (m, 2H), 7.59 (br s, 1H), 7.70-7.76 (m, 3H), 7.88 (br s, 1H), 8.10 (d, J=8.8 Hz, 2H), 8.88-8.97 (m, 2H), 10.52 (s, 1H). MS (ES+): m/z 497 (M+H)⁺.

### Example 119

### [4-([1,4]Diazepane-1-sulfonyl)-phenyl]-{5-[2-(6-trifluoromethyl-1H-benzotriazol-4-yl)-vinyl]-pyrimidin-2-yl}-amine (Compound LV)

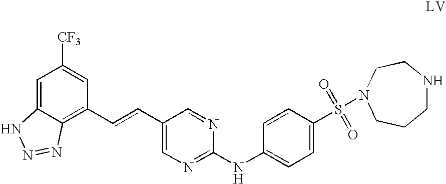

A mixture of 3-bromo-4,5-diamino-benzotrifluoride (198 mg, 0.78 mmol), intermediate 31 (300 mg, 0.65 mmol), Pd(OAc)₂ (3.0 mg, 0.013 mmol), triphenyl phosphine (13 mg, 0.052 mmol) and KHCO₃ (130 mg, 1.3 mmol) in Dioxane-DMF (1:2; 10 mL) mixture was sealed in a microwave reaction vial and microwave-irradiated at 180° C. for 30 min. The reaction mixture was purified by HPLC to give an intermediate, 4-(4-{5-[2-(2,3-diamino-5-trifluoromethyl-phenyl)-vinyl]-pyrimidin-2-ylamino}-benzenesulfonyl)-[1,4]diazepane-1-carboxylic acid tert-butyl ester (157 mg, 53% based on starting material recovered). This diamino intermediate (157 mg, 0.24 mmol) was suspended in methanol (5 mL) and conc. HCl (100 μl) was added. The mixture was treated with aqueous sodium nitrite solution (145 mg in 500 μL of water, 2.1 mmol) in two portions about five minutes apart. The reaction mixture was stirred for another ten minutes at room temperature, when effervescence subsides. The solvent evaporated to dryness, and the residue was purified by HPLC to give a yellow solid. The solid was dissolved in methanol (5 mL) and treated with methanolic HCl (1.25 M, 5 mL) for 16 h. The solvent was evaporated, the residue triturated with methanol-ethyl acetate-hexanes to give the title compound as yellow colored solid (11 mg, 8%).

¹H NMR (500 MHz, DMSO-d₆): δ 1.95-2.04 (m, 2H), 3.15-3.23 (m, 4H), 3.29 (dd, J=6.1, 5.9 Hz, 2H), 3.45-3.50 (m, 3H), 7.76 (d, J=8.9 Hz, 2H), 7.80 (d, J=16.7 Hz, 1H), 7.87 (br s, 1H), 8.96 (s, 2H), 9.0 (br s, 2H), 10.48 (s, 1H). MS (ES+): m/z 545 (M+H)⁺.

### Example 120

### [4-([1,4]Diazepane-1-sulfonyl)-phenyl]-{5-[2-(6-chloro-1H-benzotriazol-4-yl)-vinyl]-pyrimidin-2-yl}-amine (Compound LVI)

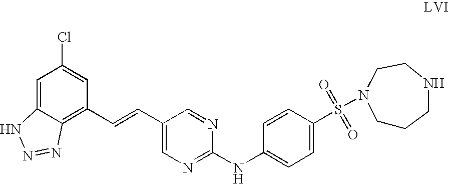

A mixture of 3-bromo-5-chlorobenzene-1,2-diamine (173 mg, 0.78 mmol), intermediate 31 (300 mg, 0.65 mmol), Pd₂(dba)₃ (59 mg, 0.065 mmol), tributyl phosphine (2.6 mL, 0.26 mmol, 1M toluene solution) and Cs₂CO₃ (422 mg, 1.3 mmol) in dioxane (12 mL) was sealed in a microwave reaction vial and irradiated with microwave at 180° C. for 30 min. The reaction mixture was purified by HPLC to give an intermediate, 3-((E)-2-(2-(4-(piperidin-4-ylsulfonyl)phenylamino)pyrimidin-5-yl)vinyl)-5-chloro-benzene-1,2-diamine (97 mg, 16%) as yellow solid. This diamino intermediate (97 mg, 0.16 mmol) was suspended in methanol (5 mL) and conc. HCl (200 μl) was added. The mixture was treated with aqueous sodium nitrite solution (220 mg in 3 mL of water, 3.2 mmol) in two portions about five minutes apart. The reaction mixture was stirred for another 1 h at room temperature. The solvent was evaporated to dryness, and the residue was purified by HPLC to give a yellow solid. The solid was suspended in methanol (5 mL) and treated with methanolic HCl (6 mL, 1.25 M solution in two portions, second portion added after 5 h) for 16 h at room temperature. The solvent was evaporated, and the sticky material was triturated with methanol-ethyl acetate hexanes to give a yellow solid (7 mg, 9%).

¹H NMR (500 MHz, DMSO-d₆): δ 1.92-2.04 (m, 2H), 3.10-3.22 (m, 4H), 3.29 (t, J=6.0, Hz, 2H), 3.45-3.53 (m, 3H), 7.66-7.77 (m, 2H), 7.76 (d, J=8.5 Hz, 2H), 8.05 (d, J=8.9 Hz, 2H), 8.88 (br s, 2H), 8.94 (s, 2H), 9.0 (br s, 2H), 10.47 (s, 1H). MS (ES+): m/z 511 (M+H)⁺.

### Example 121

### 6-Bromo-1,2,4-triazin-3-amine (Intermediate 63)

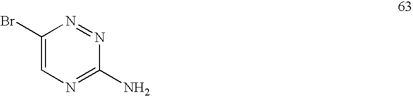

Bromine (2.14 g, 13 mmol) in chloroform (10 ml) was added to a suspension of 3-amino-1,2,4-triazine (1.07 g, 11 mmol) in chloroform (50 mL) at room temperature. The reaction mixture was stirred at room temperature for overnight. The mixture was washed by sat NaHCO₃ (1×50 mL). The chloroform layer was separated, dried over Na₂SO₄ and the solvent was removed by rotovap. The crude was washed by acetone/hexanes (v/v 1:1) to give a yellow solid (0.12 g, 12%).

¹H NMR (500 MHz, DMSO-d₆): δ 7.47 (br s, 2H), 8.40 (s, 1H).

### Example 122

### 6-(3-Methoxystyryl)-1,2,4-triazin-3-amine (Intermediate 64)

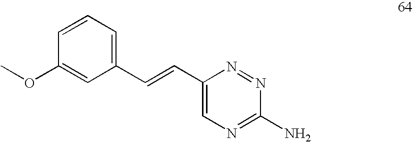

The mixture of intermediate 63 (0.1 g, 0.57 mmol), trans-2-(3-methoxyphenyl)vinylboronic acid (0.15 g, 0.84 mmol), ethylene glycol dimethyl ether (10 mL), EtOH (1 mL), H₂O (1 mL), Pd(PPh₃)₄ (70 mg, 0.06 mmol) and Na₂CO₃ (0.6 g, 5.66 mmol) was degassed with argon for 2 minutes and then refluxed for 4 hours under argon. The reaction mixture was brought to room temperature, and the solid was removed by filtration. After evaporation of the volatiles, the crude intermediate was purified by silica gel column with CHCl₃ to 10% CH₃OH/CHCl₃ as eluents to give a yellow solid (0.12 g, 92%).

### Example 123

### 4-{6-[2-(3-Methoxy-phenyl)-vinyl]-[1,2,4]triazin-3-ylamino}-N-(2-pyrrolidin-1-yl-ethyl)-benzenesulfonamide (Intermediate 65)

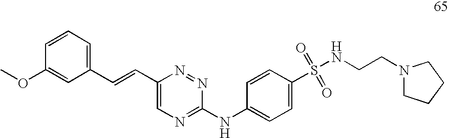

A mixture of intermediate 64 (0.12 g, 0.53 mmol), 4-bromo-N-(2-pyrrolidin-1-yl-ethyl)-benzenesulfonamide (0.26 g, 0.78 mmol), Cs₂CO₃ (0.51 g, 1.56 mmol), Xantphos (60 mg, 0.1 mmol), Pd₂(dba)₃, (50 mg, 0.05 mmol) in anhydrous dioxane (10 mL) was degassed with argon for 5 minutes and was refluxed for 2.5 h. The reaction mixture was brought to room temperature and the solvent was removed under reduced pressure. The crude intermediate was purified by silica gel column chromatography with CHCl₃ to 30% CH₃OH/CHCl₃ as eluents. The solid was washed with acetone to give a bright yellow solid (0.15 g, 59%).

¹H NMR (500 MHz, DMSO-d₆): δ 1.52-1.60 (m, 4H), 2.21-2.32 (m, 4H), 2.41 (t, J=7.2 Hz, 2H), 2.83 (t, J=6.7 Hz, 2H), 3.82 (s, 3H), 6.93 (dd, J=7.7, 1.8 Hz, 1H), 7.21-7.32 (m, 2H), 7.35 (t, J=8.0 Hz, 1H), 7.40 (br s, 1H), 7.42 (d, J=16.6 Hz, 1H), 7.69 (d, J=16.6 Hz, 1H), 7.77 (d, J=9 Hz, 2H), 7.97 (d, J=8.9 Hz, 2H), 8.90 (s, 1H), 10.69 (s, 1H).

### Example 124

### 4-{6-[2-(3-Hydroxy-phenyl)-vinyl]-[1,2,4]triazin-3-ylamino}-N-(2-pyrrolidin-1-yl-ethyl)-benzenesulfonamide (Compound LVII)

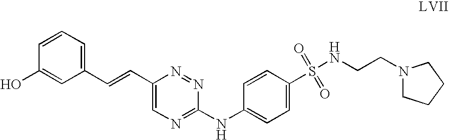

Intermediate 65 (0.1 g, 0.21 mmol) in CHCl₃ (10 mL) was stirred with 1M BBr₃ in CH₂Cl₂ (10 mL, 10 mmol) at room temperature for 24 h. The reaction was quenched by methanol (1 mL). After removing the solvent, the crude residue was dissolved in EtOAc (150 mL), washed by saturated NaHCO₃ (2×25 mL). The organic layer was separated, dried over Na₂SO₄, and evaporated. The crude product was purified by silica gel column with CHCl₃ to 20% CH₃OH/CHCl₃ as eluents to give a yellow solid (50 mg, 51%).

¹H NMR (500 MHz, DMSO-d₆): δ 1.51-1.65 (m, 4H), 2.31-2.40 (m, 4H), 2.41 (t, J=7.0 Hz, 2H), 2.83 (t, J=6.5 Hz, 2H), 6.76 (dd, J=7.9, 1.7 Hz, 1H), 7.05 (s, 1H), 7.12 (d, J=7.7 Hz, 1H), 7.23 (t, J=7.8 Hz, 1H), 7.28 (d, J=16.7 Hz, 1H), 7.39 (br s, 1H), 7.63 (d, J=16.6 Hz, 1H), 7.77 (d, J=8.9 Hz, 2H), 7.80 (d, J=8.9 Hz, 2H), 8.92 (s, 1H), 9.55 (s, 1H), 10.66 (s, 1H). MS (ES+): m/z 467 (M+H)⁺.

### Example 125

### 4-{5-[2-(3-Methoxy-phenyl)-vinyl]-pyrimidin-2-ylamino}-N-(2-pyrrolidin-1-yl-ethyl)-benzenesulfonamide (Intermediate 66)

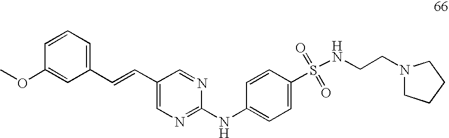

To a solution of intermediate 13 (0.12 g, 0.53 mmol), 4-bromo-N-(2-pyrrolidin-1-yl-ethyl)-benzenesulfonamide (0.28 g, 0.84 mmol) in anhydrous dioxane (15 mL) were added Cs₂CO₃ (0.52 g, 1.60 mmol), Xantphos (60 mg, 0.1 mmol), Pd₂(dba)₃ (50 mg, 0.06 mmol). The reaction mixture was degassed with argon for 2 min. and refluxed for 1 h. The solvent was removed by rotovap and the crude intermediate was purified by silica gel column with CHCl₃ to 20% CH₃OH in CHCl₃ as eluents to give a white solid (0.24 g, 94%).

### Example 126

### 4-{5-[2-(3-Hydroxy-phenyl)-vinyl]-pyrimidin-2-ylamino}-N-(2-pyrrolidin-1-yl-ethyl)-benzenesulfonamide (Compound LVIII)

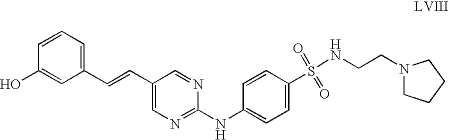

A suspension of intermediate 66 (0.1 g, 0.21 mmol) in CHCl₃ (10 mL) was added 1 M BBr₃ in CH₂Cl₂ (10 mL, 10 mmol) at room temperature. The reaction mixture was stirred at room temperature for overnight. The mixture was quenched with methanol (1 mL). The solvent was removed by rotovap and crude product was purified by silica gel column with CHCl₃ to 20% CH₃OH/CHCl₃ as eluents to give an off-white solid after washed by CHCl₃ (20 mg, 21%).

¹H NMR (500 MHz, DMSO-d₆): δ 1.64 (br s, 4H), 2.41 (br s, 4H), 2.83 (br s, 2H), 6.69 (d, J=7.9 Hz, 1H), 6.96 (s, 1H), 7.01 (d, J=7.5 Hz, 1H), 7.07 (d, J=16.6 Hz, 1H), 7.18 (t, J=7.9 Hz, 1H), 7.24 (d, J=16.6 Hz, 1H), 7.37 (br s, 1H), 7.72 (d, J=8.8 Hz, 2H), 7.97 (d, J=8.7 Hz, 2H), 8.82 (s, 1H), 9.46 (s, 1H), 10.26 (s, 1H). MS (ES+): m/z 467 (M+H)⁺.

### Example 127

### 2-(Bromomethyl)-1-chloro-4-methoxybenzene (Intermediate 67)

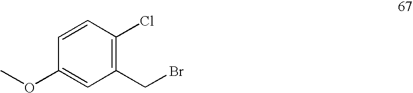

To a solution of 1-chloro-4-methoxy-2-methylbenzene (4.1 g, 26.2 mmol) in carbon tetrachloride (60 mL) were added N-bromosuccinimide (NBS, 4.7 g, 26.4 mmol) and 2,2′-azobis(2-methylpropionitrile) (AIBN, 0.9 g, 5.5 mmol). The resulting mixture was refluxed for 3 h and cooled to room temperature. Insoluble succinimide was filtered off and the solution was concentrated. The crude oil was used for next reaction without further purification.

### Example 128

### (2-Chloro-5-methoxy-benzyl)-triphenyl-phosphonium bromide (Intermediate 68)

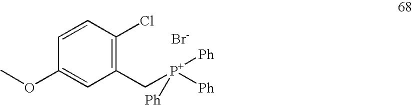

A solution of intermediate 67 and triphenylphosphine (6.87 g, 26.2 mmol) in anhydrous toluene (50 mL) was refluxed for 1 h. The resulting precipitate was collected by filtration, washed by acetone, and dried under vacuum to provide the intermediate as an off-white solid (8 g, 62% in 2-steps).

¹H NMR (500 MHz, DMSO-d₆): δ 3.49 (s, 3H), 5.11 (d, J=15.1 Hz, 2H), 6.64 (t, J=2.8 Hz, 1H), 6.80-7.00 (m, 1H), 7.29 (d, J=8.9 Hz, 1H), 7.60-7.70 (m, 6H), 7.71-7.80 (m, 6H), 7.91-8.02 (m, 3H).

### Example 129

### 1-Chloro-4-methoxy-2-vinylbenzene (Intermediate 69)

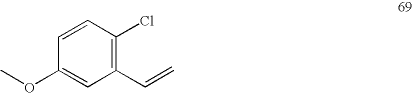

A mixture of intermediate 68 (1.84 g, 3.7 mmol) and 37 wt. % formaldehyde (0.9 g, 11 mmol) were suspended in 1N NaOH (20 mL) and heated at 75° C. for 1 h. The mixture was allowed to cool to room temperature and the resulting solution was extracted with CHCl₃ (2×30 mL) and the organic layers were separated. The organic layers were combined and washed with brine, dried over Na₂SO₄ and filtered. The filtrate was concentrated in vacuo and the crude intermediate was purified by flash chromatography on silica gel with hexanes to chloroform as eluents to afford the intermediate as colorless oil (0.5 g, 80%).

¹H NMR (500 MHz, DMSO-d₆): δ 3.79 (s, 3H), 5.45 (dd, J=11.3, 0.8 Hz, 1H), 5.94 (dd, J=17.7, 0.8 Hz, 1H), 6.80-6.91 (m, 1H), 6.91-7.00 (m, 1H), 7.22 (d, J=3.1 Hz, 1H), 7.34 (d, J=8.8 Hz, 1H).

### Example 130

### 5-(2-Chloro-5-methoxystyryl)pyrimidin-2-amine (Intermediate 70)

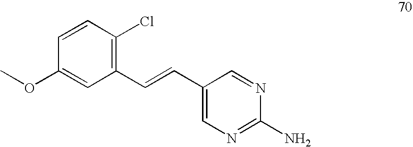

A mixture of intermediate 69 (0.45 g, 2.7 mmol), 5-bromopyrimidin-2-amine (0.23 g, 1.3 mmol), Pd(OAc)₂ (6 mg, 0.03 mmol), PPh₃ (28 mg, 0.11 mmol) and NaHCO₃ (0.22 g, 2.6 mmol) were suspended in DMF (10 mL) and degassed with argon for 2 minutes. The mixture was heated at reflux under the argon atmosphere for 2 h. After cooling down, the mixture was poured into water (80 mL) and the dark yellow precipitate was collected by filtration. The crude product was used for next reaction without further purification.

¹H NMR (500 MHz, DMSO-d₆): δ 3.81 (s, 3H), 6.86 (dd, J=17.7, 3.0 Hz, 1H), 6.91 (br s, 2H), 7.14 (d, J=16.5 Hz, 1H), 7.22 (d, J=16.5 Hz, 1H), 7.32 (d, J=3 Hz, 1H), 7.35 (d, J=9 Hz, 1H), 8.52 (s, 2H).

### Example 131

### 4-{5-[2-(2-Chloro-5-methoxy-phenyl)-vinyl]-pyrimidin-2-ylamino}-N-(2-pyrrolidin-1-yl-ethyl)-benzenesulfonamide (Intermediate 71)

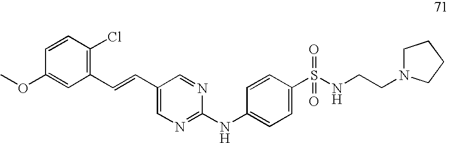

A mixture of intermediate 70, 4-amino-N-(2-pyrrolidin-1-yl-ethyl)-benzenesulfonamide (0.66 g, 2.0 mmol), Pd₂(dba)₃ (0.12 g, 0.13 mmol), Xantphos (0.15 g, 0.26 mmol) and Cs₂CO₃ (0.87 g, 2.7 mmol) were suspended in dioxane (20 mL) and degassed with argon for 2 minutes. The mixture was heated at reflux under the argon atmosphere for 2 h. After cooling down, the mixture was poured into water (30 mL) and extracted with EtOAc (60 mL). The organic layer was separated and washed with brine, dried over Na₂SO₄ and filtered. The filtrate was concentrated in vacuo and the crude intermediate was purified by silica gel with CHCl₃ to 30% MeOH/CHCl₃ as eluents to afford the intermediate as a pale yellow solid (0.1 g, 15% in 2-steps).

¹H NMR (500 MHz, DMSO-d₆): δ 1.88 (br s, 4H), 2.90-3.05 (m, 4H), 3.20 (br s, 2H), 3.83 (s, 3H), 6.91 (dd, J=8.8, 3.0 Hz, 1H), 7.30 (d, J=16.5 Hz, 1H), 7.31-7.40 (m, 3H), 7.75 (d, J=8.8 Hz, 2H), 8.01 (d, J=8.9 Hz, 2H), 8.87 (s, 2H), 9.63 (br s, 1H), 10.4 (br s, 1H).

### Example 132

### 4-{5-[2-(2-Chloro-5-hydroxy-phenyl)-vinyl]-pyrimidin-2-ylamino}-N-(2-pyrrolidin-1-yl-ethyl)-benzenesulfonamide (Compound LIX)

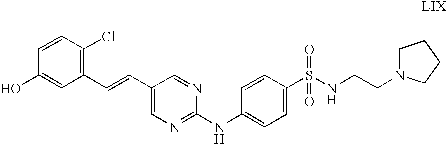

A solution of intermediate 71 (100 mg, 0.19 mmol) in 1M BBr₃/CH₂Cl₂ (10 mL) was stirred at room temperature for overnight. The reaction was quenched with saturated NaHCO₃ solution until the pH-7 and the mixture extracted with EtOAc (3×40 mL). The organic layer was separated and washed with brine, dried over Na₂SO₄ and filtered. The crude product was purified by silica gel with CHCl₃ to 30% MeOH/CHCl₃ as eluents to afford the product as a yellow solid after washed by acetone (30 mg, 31%).

¹H NMR (500 MHz, DMSO-d₆): δ 1.61-1.70 (m, 4H), 2.31-2.42 (m, 4H), 2.41 (t, J=7.2 Hz, 2H), 2.82 (t, J=7.0 Hz, 2H), 6.74 (dd, J=8.8, 3.0 Hz, 1H), 7.11 (d, J=16.5 Hz, 1H), 7.18 (d, J=3.0 Hz, 1H), 7.35 (br s, 1H), 7.40 (d, J=16.5 Hz, 1H), 7.71 (d, J=8.9 Hz, 2H), 7.98 (d, J=8.9 Hz, 2H), 8.85 (s, 2H), 9.75 (br s, 1H), 10.33 (s, 1H)

MS(ES+): m/z 500 (M+H)⁺.

### Example 133

### 4-{5-[2-(2-Chloro-5-hydroxy-phenyl)-ethyl]-pyrimidin-2-ylamino}-N-(2-pyrrolidin-1-yl-ethyl)-benzenesulfonamide (Compound LX)

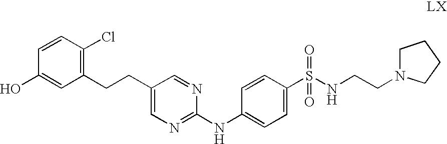

The above-described compound LIX (50 mg, 0.1 mmol) was dissolved in MeOH (20 mL), EtOAc (20 mL) and AcOH (1 mL) and was degassed with argon for 2 min. Pd/C (10% by wt, 50 mg) was added to the reaction and filled with hydrogen balloon and stirred at room temperature for overnight. The Pd/C was removed by filtration and washed with MeOH and concentrated under reduced pressure resulted in The title compound an off-white solid after washed by acetone (22 mg, 44%).

¹H NMR (500 MHz, DMSO-d₆): δ 1.80-1.88 (m, 2H), 1.91-2.00 (m, 2H), 2.70-2.81 (m, 2H), 2.81-2.88 (m, 2H), 2.91-3.00 (m, 2H), 3.00-3.08 (m, 2H), 3.10-3.20 (m, 2H), 3.40-3.55 (m, 2H), 6.64 (dd, J=8.7 2.9 Hz, 1H), 6.77 (d, J=2.7 Hz, 1H), 7.18 (d, J=8.7 Hz, 1H), 7.73 (d, J=9.0 Hz, 2H), 7.84 (t, J=6.1 Hz, 1H), 7.96 (d, J=8.9 Hz, 1H), 8.37 (s, 2H), 10.09 (s, 1H), 10.25 (br s, 1H). MS (ES+): m/z 502 (M+H)⁺.

### Example 134

### 4-{5-[2-(2-Fluoro-5-hydroxy-phenyl)-vinyl]-pyrimidin-2-ylamino}-N-(2-pyrrolidin-1-yl-ethyl)-benzenesulfonamide (Compound LXI)

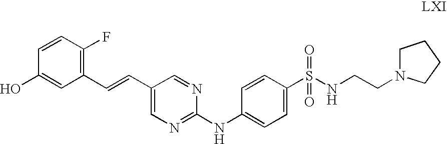

To a solution of 1-fluoro-4-methoxy-2-methylbenzene (7.0 g, 49.9 mmol) in CCl₄ (80 mL) were added N-bromosuccinimide (NBS, 9.8 g, 55.0 mmol) and 2,2′-azobis(2-methylpropionitrile) (AIBN, 0.8 g, 5.0 mmol). The resulting mixture was refluxed for 4 h and cooled to room temperature. Insoluble succinimide was filtered off and the solution was concentrated. The crude oil was used for next reaction without further purification.

A solution of the above prepared 2-(bromomethyl)-1-fluoro-4-methoxybenzene and triphenylphosphine (14.4 g, 54.9 mmol) in anhydrous toluene (50 mL) was refluxed for 1 h. The resulting precipitate was collected by filtration, washed by acetone, and dried under vacuum to provide the corresponding (2-fluoro-5-methoxy-benzyl)-triphenyl-phosphonium bromide as an off-white solid (15 g, 62%).

A mixture of (2-fluoro-5-methoxy-benzyl)-triphenyl-phosphonium bromide (6.9 g, 14.3 mmol) and 37 wt. % formaldehyde (5.8 g, 71.5 mmol) were suspended in 1N NaOH (60 mL) and heated at 80° C. for 2.5 h. The mixture was allowed to cool to room temperature and the resulting solution was extracted with EtOAc (2×30 mL) and the organic layer separated. The organic layers were combined and washed with brine, dried over Na₂SO₄ and filtered. The filtrate was concentrated in vacuo and the crude product was purified by flash chromatography on silica gel with hexanes to CHCl₃ as eluents to afford 1-fluoro-4-methoxy-2-vinylbenzene as colorless oil (1.2 g, 55%).

A mixture of 1-fluoro-4-methoxy-2-vinylbenzene (0.78 g, 4.6 mmol), 5-bromopyrimidin-2-amine (0.85 g, 5.6 mmol), Pd(OAc)₂ (20 mg, 0.09 mmol), PPh₃ (94 mg, 0.36 mmol) and sodium bicarbonate (0.75 g, 8.93 mmol) were suspended in DMF (40 mL) and degassed with argon for 2 minutes. The mixture was heated at reflux under the argon atmosphere for 2 h. After cooling down, the mixture was poured into water (80 mL) and the yellow precipitate was collected by filtration. The crude product was used for next reaction without further purification.

A mixture of 5-(2-fluoro-5-methoxystyryl)pyrimidin-2-amine (0.40 g, 1.63 mmol), 4-amino-N-(2-pyrrolidin-1-yl-ethyl)-benzenesulfonamide (0.65 g, 2.0 mmol), Pd₂(dba)₃ (0.15 g, 0.16 mmol), Xantphos (0.19 mg, 0.33 mmol) and cesium carbonate (1.60 g, 4.91 mmol) were suspended in dioxane (50 mL) and degassed with argon for 2 minutes. The mixture was heated at reflux under the argon atmosphere for 2 h. After cooling down, the mixture was poured into water (30 mL) and extracted with EtOAc (60 mL). The organic layer was separated and washed with brine, dried over Na₂SO₄ and filtered. The filtrate was concentrated in vacuo and the crude product was purified by flash chromatography on silica gel with CHCl₃ to 20% MeOH/CHCl₃ as eluents to afford the product as a pale yellow solid.

A solution of 4-{5-[2-(2-fluoro-5-methoxy-phenyl)-vinyl]-pyrimidin-2-ylamino}-N-(2-pyrrolidin-1-yl-ethyl)-benzenesulfonamide in 1 M BBr₃/CH₂Cl₂ (10 mL) was stirred at room temperature for overnight. The reaction was quenched with saturated NaHCO₃ solution until the pH-7 and the mixture extracted with EtOAc (3×40 mL). The organic layer was separated and washed with brine, dried over Na₂SO₄ and filtered. The crude product was purified by flash chromatography on silica gel with CHCl₃ to 40% MeOH/CHCl₃ as eluents to afford the product as an off-white solid (100 mg, 13% in 2-steps).

¹H NMR (500 MHz, DMSO-d₆): δ 1.55-1.65 (m, 4H), 2.30-2.42 (m, 4H), 2.41 (t, J=7.2 Hz, 2H), 2.82 (t, J=7.0 Hz, 2H), 6.74 (dd, J=8.8, 3.0 Hz, 1H), 7.11 (d, J=16.5 Hz, 1H), 7.18 (d, J=3.0 Hz, 1H), 7.35 (br s, 1H), 7.40 (d, J=16.5 Hz, 1H), 7.71 (d, J=8.9 Hz, 2H), 7.98 (d, J=8.9 Hz, 2H), 8.85 (s, 2H), 9.75 (br s, 1H), 10.33 (s, 1H). MS(ES+): m/z 500 (M+H)⁺.

### Example 135

### 5-Bromo-4-methylpyrimidin-2-amine (Intermediate 72)

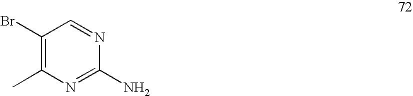

Bromine (6.86 g, 42.89 mmol) was added slowly to a solution of 4-methylpyrimidin-2-amine (4.24 g, 38.85 mmol) in THF (45 mL) and water (45 mL) at room temperature. The mixture was stirred at room temperature for 2 h. The solvent was removed by rotovap and the solid was collected by filtration, washed by acetone to yield an off-white solid (5 g, 68%). ¹H NMR (500 MHz, DMSO-d₆): δ 2.39 (s, 3H), 6.60 (br s, 2H), 8.39 (s, 1H).

### Example 136

### 5-(3-Methoxystyryl)-4-methylpyrimidin-2-amine (Intermediate 73)

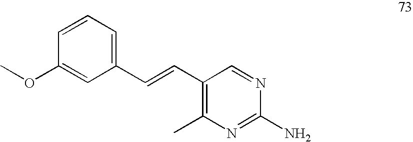

A mixture of intermediate 72 (0.75 g, 3.97 mmol), 1-methoxy-3-vinylbenzene (0.80 g, 5.96 mmol), Pd(OAc)₂ (18 mg, 0.08 mmol), PPh₃ (84 mg, 0.32 mmol) and NaHCO₃ (0.67 g, 8.00 mmol) in DMF (40 mL) was degassed with argon for 2 minutes and then refluxed at 160° C. for 4 h. After cooling to room temperature, the mixture was diluted with water (100 mL). The precipitate was collected by filtration and washed by water to yield grey solid (0.6 g, 63%).

### Example 137

### 4-{5-[2-(3-Methoxy-phenyl)-vinyl]-4-methyl-pyrimidin-2-ylamino}-N-(2-pyrrolidin-1-yl-ethyl)-benzenesulfonamide (Intermediate 74)

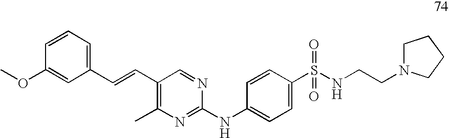

A mixture of intermediate 73 (0.23 g, 0.95 mmol), 4-bromo-N-(2-pyrrolidin-1-yl-ethyl)-benzenesulfonamide (0.40 g, 1.20 mmol), Pd₂(dba)₃ (87 mg, 0.09 mmol), Xantphos (0.11 g, 0.19 mmol) and Cs₂CO₃ (0.93 g, 2.85 mmol) in dioxane (30 mL) was degassed with Ar for 2 minutes and then refluxed at 110° C. for 3 h. After cooling to room temperature, the solvent was removed by rotovap. The crude product was suspended in water (100 mL) and extracted by EtOAc (2×75 mL). The combined EtOAc was dried over Na₂SO₄, filtration to remove the salt. The solvent was removed by rotovap. The solid was washed by acetone to yield The title intermediate as an off-white solid (0.2 g, 43%).

¹H NMR (500 MHz, DMSO-d₆): δ 1.58-1.65 (m, 4H), 2.30-2.35 (m, 4H), 2.41 (t, J=7.3 Hz, 2H), 2.57 (s, 3H), 2.81 (t, J=6.9 Hz, 1H), 3.80 (s, 3H), 6.80-6.90 (m, 1H), 7.15 (d, J=16.4 Hz, 1H), 7.20 (d, J=7.5 Hz, 2H), 7.25-7.45 (m, 3H), 7.69 (d, J=7.8 Hz, 2H).

### Example 138

### 4-{5-[2-(3-Hydroxy-phenyl)-vinyl]-4-methyl-pyrimidin-2-ylamino}-N-(2-pyrrolidin-1-yl-ethyl)-benzenesulfonamide (Compound LXII)

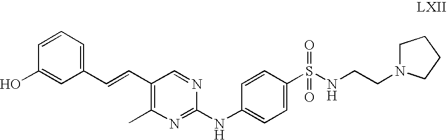

A solution of intermediate 74 (0.2 g, 0.41 mmol) in 1M BBr₃/CH₂Cl₂ (5 mL) was stirred at room temperature for 22 h. The reaction was quenched with saturated NaHCO₃ solution until the pH-7 and the mixture extracted with CHCl₃ (3×50 mL). The organic layer was separated and washed with brine, dried over Na₂SO₄ and filtered. The crude product was purified by flash chromatography on silica gel with CHCl₃ to 10% MeOH/CHCl₃ as eluents to afford the title product as a white solid (0.1 g, 51%).

¹H NMR (500 MHz, DMSO-d₆): δ 1.60-1.71 (m, 4H), 2.30-2.45 (m, 6H), 2.55 (s, 3H), 2.80-2.89 (m, 2H), 6.69 (dd, J=8.7, 3.1 Hz, 1H), 6.99 (d, J=1.8 Hz, 1H), 7.05 (d, J=7.8 Hz, 1H), 7.08 (d, J=16.5 Hz, 1H), 7.19 (s, 2H), 7.18 (d, J=16.5 Hz, 1H), 7.33 (br s, 1H), 7.70 (d, J=9.0 Hz, 2H), 7.99 (d, J=9.0 Hz, 2H), 8.80 (s, 1H), 9.43 (s, 1H), 10.16 (s, 1H). MS(ES+): m/z 480 (M+H)⁺.

### Example 139

### m-Tolyl benzoate (Intermediate 75)

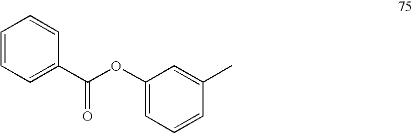

Benzoyl chloride (8.94 g, 63.60 mmol) was added slowly into a solution of m-cresol (5.5 g, 50.86 mmol), 4-methylmorpholine (10.3 g, 0.10 mol) in anhydrous CH₂Cl₂ (100 mL). The mixture was heated to reflux for 3 h. After cooling to room temperature, the solution was washed by water (2×35 mL), 2 M NaOH (1×35 mL) and saturated NaHCO₃ (1×35 mL), respectively. The organic phase was dried over Na₂SO₄ and the salt was removed by filtration. The solvent was removed by rotovap to yield pale brown oil (5.3 g, 49%).

### Example 140

### 3-((E)-2-(2-Aminopyrimidin-5-yl)vinyl)phenyl benzoate (Intermediate 76)

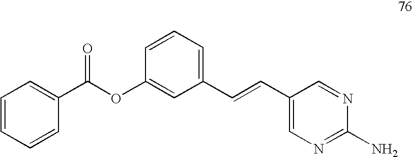

To a solution of intermediate 75 (5.3 g, 24.92 mmol) in CCl₄ (100 mL) were added N-bromosuccinimide (NBS, 5.0 g, 28.1 mmol) and 2,2′-azobis(2-methylpropionitrile) (AIBN, 0.4 g, 2.4 mmol). The resulting mixture was refluxed for overnight and cooled to room temperature. Insoluble succinimide was filtered off and the solution was concentrated. The trace precipitate was removed by filtration with hexanes. The hexanes solvent was removed by rotovap to yield oil. The product was used for next reaction without further purification.

The above product was dissolved in toluene (80 mL) and PPh₃ (5.22 g, 19.9 mmol) was added into the solution. The mixture was refluxed for 30 min. The precipitate was collected by filtration and washed thoroughly by acetone to yield a pale yellow solid (3.9 g, 28% in 2-steps).

The above phosphonium salt (3.0 g, 5.4 mmol) and 37 wt. % formaldehyde (2.2 g, 27.1 mmol) were suspended in 1N NaOH (55 mL) and heated at 60° C. for 30 min. The mixture was allowed to cool to room temperature and the resulting solution was extracted with CHCl₃ (2×30 mL) and the organic layer separated. The organic layers were combined and washed with brine, dried over Na₂SO₄ and filtered. The filtrate was concentrated in vacuo and the crude product was purified by flash chromatography on silica gel with hexanes to chloroform as eluents to afford 3-vinylphenyl benzoate as pale yellow oil (1.1 g, 90%).

A mixture of 5-bromo-pyrimidin-2-amine (0.40 g, 2.3 mmol), 3-vinylphenyl benzoate (0.65 g, 2.9 mmol), Pd(OAc)₂ (10 mg, 0.04 mmol), PPh₃ (48 mg, 0.18 mmol) and NaHCO₃ (0.39 g, 4.64 mmol) in DMF (25 mL) was degassed with argon for 2 minutes and then refluxed at 160° C. for 1 h. After cooling to room temperature, the mixture was diluted with water (50 mL) to get a dark yellow precipitate. The solid was collected by filtration and washed by water. The crude was purified by silica gel column with CHCl₃ to 10% CH₃OH/CHCl₃ as eluents. The product was further purified by washing with CHCl₃ to yield a yellow solid (0.47 g, 64%).

¹H NMR (500 MHz, DMSO-d₆): δ 6.84 (br s, 2H), 7.05-7.2 (m, 3H), 7.41-7.50 (m, 3H), 7.62-7.70 (m, 2H), 7.73-7.81 (m, 1H), 8.12-8.20 (m, 2H), 8.50 (s, 2H).

### Example 141

### Benzoic acid 3-(2-{2-[4-(2-pyrrolidin-1-yl-ethylsulfamoyl)-phenylamino]-pyrimidin-5-yl}-vinyl)-phenyl ester (Compound LXIII)

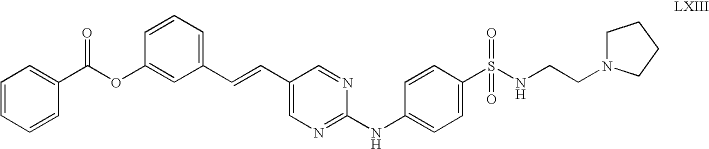

A mixture of intermediate 76 (0.21 g, 0.66 mmol), 4-bromo-N-(2-pyrrolidin-1-yl-ethyl)-benzenesulfonamide (0.40 g, 1.20 mmol), Pd₂(dba)₃ (61 mg, 0.07 mmol), Xantphos (77 mg, 0.13 mmol) and Cs₂CO₃ (0.65 g, 2.00 mmol) in dioxane (40 mL) was degassed with Ar for 2 minutes and then refluxed at 110° C. for 2 h. After cooling to room temperature, the solvent was removed by rotovap. The crude product was suspended in water (50 mL) and extracted by EtOAc (2×75 mL). The combined EtOAc was dried over Na₂SO₄, filtration to remove the salt. The solvent was removed by rotovap. The solid was purified by silica gel column with 30% CH₃OH/CHCl₃ as an eluent. The product was further purified by washing with acetone/hexanes (v/v 3:1) to yield a pale yellow solid (0.1 g, 27%).

¹H NMR (500 MHz, DMSO-d₆): δ 1.55-1.65 (m, 4H), 2.23-2.35 (m, 4H), 2.41 (t, J=7.2 Hz, 2H), 2.75-2.85 (m, 2H), 7.15-7.25 (m, 1H), 7.27 (d, J=16.5 Hz, 1H), 7.34 (br s, 1H), 7.37 (d, J=16.6 Hz, 1H), 7.49 (d, J=5.6 Hz, 2H), 7.54 (s, 1H), 7.64 (t, J=7.8 Hz, 2H), 7.71 (d, J=8.8 Hz, 1H), 7.78 (t, J=7.4 Hz, 1H), 7.97 (d, J=8.8 Hz, 2H), 8.17 (d, J=7.4 Hz, 2H), 8.83 (s, 2H), 10.29 (s, 1H). MS (ES+): m/z 570 (M+H)⁺.

### Example 142

### 2-Fluoro-5-(2-{2-[4-(piperidine-4-sulfonyl)-phenylamino]-pyrimidin-5-yl}-vinyl)-phenol (Compound LXIV)

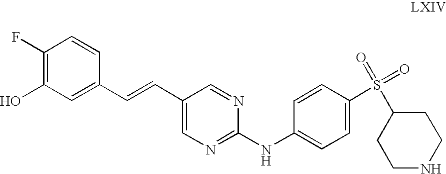

To a solution of 1-fluoro-2-methoxy-4-methylbenzene (5.0 g, 29.92 mmol) in carbon tetrachloride (60 mL) were added N-bromosuccinimide (NBS, 5.86 g, 32.92 mmol) and 2,2′-azobis(2-methylpropionitrile) (AIBN, 0.49 g, 3.0 mmol). The resulting mixture was refluxed for 2.5 h and cooled to room temperature. Insoluble succinimide was filtered off and the solution was concentrated. The crude oil was used for next reaction without further purification.

A solution of the above prepared 4-(bromomethyl)-1-fluoro-2-methoxybenzene and triphenylphosphine (8.63 g, 32.90 mmol) in anhydrous toluene (80 mL) was refluxed for 2 h. The resulting precipitate was collected by filtration, washed by hexanes, and dried under vacuum to provide the corresponding the phosphonium salt as an off-white solid (9.6 g, 63% in 2-steps).

A mixture of (4-fluoro-3-methoxy-benzyl)-triphenyl-phosphonium bromide (9.6 g, 19.96 mmol) and 37 wt. % formaldehyde (8.1 g, 100 mmol) were suspended in 1N NaOH (75 mL) and heated at 80° C. for 3 h. The mixture was allowed to cool to room temperature and the resulting solution was extracted with EtOAc (2×30 mL) and the organic layer separated. The organic layers were combined and washed with brine, dried over Na₂SO₄ and filtered. The filtrate was concentrated in vacuo and the crude product was purified by flash chromatography on silica gel with hexanes to chloroform as eluents to afford 1-fluoro-2-methoxy-4-vinylbenzene as colorless oil (1.8 g, 59%).

A mixture of 1-fluoro-4-methoxy-2-vinylbenzene (1.3 g, 8.55 mmol), 5-bromopyrimidin-2-amine (1.14 g, 6.58 mmol), Pd(OAc)₂ (30 mg, 0.13 mmol), PPh₃ (0.14 g, 0.53 mmol) and sodium bicarbonate (1.1 g, 13.10 mmol) were suspended in DMF (40 mL) and degassed with argon for 2 minutes. The mixture was heated at reflux under the argon atmosphere for 1 h. After cooling down, the mixture was poured into water (80 mL) and the yellow precipitate was collected by filtration. The crude product was used for next reaction without further purification.

A mixture of the 5-(4-fluoro-3-methoxystyryl)pyrimidin-2-amine (0.32 g, 1.30 mmol), tert-butyl 4-(4-bromophenylsulfonyl)piperidine-1-carboxylate (0.53 g, 1.31 mmol), Pd₂(dba)₃ (0.12 g, 0.13 mmol), Xantphos (0.15 g, 0.26 mmol) and cesium carbonate (1.27 g, 3.90 mmol) were suspended in dioxane (80 mL) and degassed with argon for 2 minutes. The mixture was heated at reflux under the argon atmosphere for 2 h. After cooling down, the mixture was poured into water (30 mL) and extracted with EtOAc (60 mL). The organic layer was separated and washed with brine, dried over Na₂SO₄ and filtered. The filtrate was concentrated in vacuo and the crude product was purified by flash chromatography on silica gel with CHCl₃ to 5% MeOH/CHCl₃ as eluents to afford the product as a brown solid.

A solution of the above product in 1M BBr₃/CH₂Cl₂ (10 mL) was stirred at room temperature for overnight. The reaction was quenched with saturated NaHCO₃ solution until the pH-7 and the mixture extracted with EtOAc (3×40 mL). The organic layer was separated and washed with brine, dried over Na₂SO₄ and filtered. The crude product was purified by flash chromatography on silica gel with CHCl₃ to 40% MeOH/CHCl₃ as eluents to afford the product as an off-white solid (100 mg, 13% in 2-steps).

¹H NMR (500 MHz, DMSO-d₆): δ 1.31-1.56 (m, 2H), 1.63-1.78 (m, 2H), 2.30-2.45 (m, 2H), 2.91-3.01 (m, 2H), 3.11-3.22 (m, 1H), 4.11-4.25 (m, 1H), 6.85 (br s, 1H), 6.92 (d, J=16.6 Hz, 1H), 7.00-7.11 (m, 1H), 7.05-7.15 (m, 1H), 7.19 (d, J=16.5 Hz, 1H), 7.71 (d, J=8.8 Hz, 1H), 8.02 (d, J=8.9 Hz, 1H), 8.80 (s, 2H), 10.36 (br s, 1H). MS(ES+): m/z 455 (M+H)⁺.

### Example 143

### 5-(3-(Trifluoromethyl)benzamido)-2-chlorobenzoic acid (Intermediate 77)

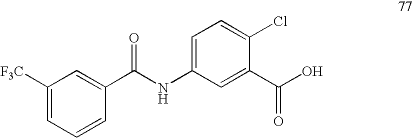

To a stirring solution of 5-amino-2-chlorobenzoic acid (1.74 g, 10.1 mmol) in anhydrous THF (60 mL), 3-(trifluoromethyl)benzoyl chloride (2.33 g, 11.2 mmol) was added slowly. The mixture was stirred at room temperature under argon for overnight. Adding more the benzoyl chloride to make sure the starting material all reacted if needed. The solvent was removed under reduced pressure and washed with acetone/chloroform (v/v 1:1) to afford the title intermediate as a white solid (1.5 g, 43%).

¹H NMR (500 MHz, DMSO-d₆): δ 7.55 (d, J=8.8 Hz, 1H), 7.80 (t, J=7.8 Hz, 1H), 7.99 (dd, J=8.7, 2.5 Hz, 2H), 8.20-8.33 (m, 2H), 8.32 (s, 1H), 10.69 (s, 1H), 13.47 (br s, 1H).

### Example 144

### N-(4-Chloro-3-(hydroxymethyl)phenyl)-3-(trifluoromethyl)benzamide (Intermediate 78)

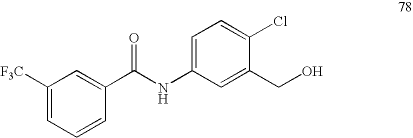

To a stirring solution of intermediate 77 (7.43 g, 21.63 mmol) in anhydrous THF (125 mL), 2.0 M LiAlH₄ in THF (21.6 mL, 43.26 mmol) was added slowly at room temperature. The mixture was stirred at reflux for 30 min under argon. After cooling to 0° C., the reaction mixture was carefully quenched by methanol (5 mL). The white solid was removed by filtration and washed by acetone thoroughly. The combined filtrate was concentrated under vacuum. The obtained solid was washed with CHCl₃ to yield The title intermediate as a white solid (2.1 g, 28%).

¹H NMR (500 MHz, DMSO-d₆): δ 4.57 (s, 2H), 5.50 (br s, 1H), 7.39 (d, J=8.7 Hz, 1H), 7.72-7.85 (m, 2H), 7.90-8.05 (m, 2H), 8.28 (d, J=7.9 Hz, 1H), 8.32 (s, 1H), 10.62 (br s, 1H).

### Example 145

### N-(4-chloro-3-vinylphenyl)-3-(trifluoromethyl)benzamide (Intermediate 79)

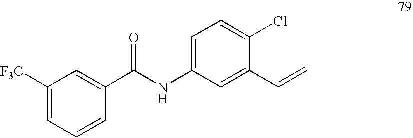

A solution of intermediate 78 (2.1 g, 6.47 mmol) and MnO₂ (2.78 g, 32.0 mmol) in anhydrous toluene (50 mL) was refluxed until all starting material was reacted. The solid was removed by filtration and washed by EtOAc. The filtrate was concentrated by rotovap and the obtained solid was washed by CHCl₃ to yield N-(4-chloro-3-formylphenyl)-3-(trifluoromethyl)benzamide as a dark brown solid (0.75 g, 35%). n-BuLi (1.6M in THF, 2.8 mL, 4.48 mmol) was added to methyltriphenylphosphonium bromide (1.7 g, 4.76 mmol) in anhydrous THF (20 mL) at room temperature.

After the solution was stirred at room temperature for 40 min, N-(4-chloro-3-formylphenyl)-3-(trifluoromethyl)benzamide (0.7 g, 2.14 mmol) was added into the solution and the mixture was refluxed for 2.5 h. After cooling down, the reaction was quenched by methanol (2 mL) and the solvent was removed by rotovap. The crude product was purified by silica gel column with CHCl₃ as an eluent to yield the intermediate as a colorless sticky oil (0.5 g, 72%).

¹H NMR (500 MHz, DMSO-d₆): δ 5.49 (d, J=11.6 Hz, 1H), 5.81 (d, J=18.1 Hz, 1H), 7.03 (dd, J=17.5, 11.0 Hz, 1H), 7.48 (d, J=8.7 Hz, 1H), 7.70-7.85 (m, 2H), 7.98 (d, J=8.6 Hz, 1H), 8.11 (d, J=2.6 Hz, 1H), 8.27 (d, J=8.6 Hz, 1H), 8.31 (s, 1H), 10.58 (s, 1H)

### Example 146

### N-(3-((E)-2-(2-aminopyrimidin-5-yl)vinyl)-4-chlorophenyl)-3-(trifluoromethyl)benzamide (Intermediate 80)

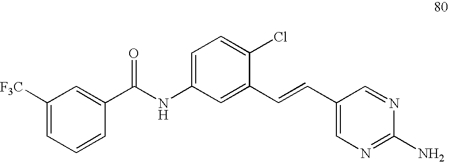

A mixture of 5-bromo-pyrimidin-2-amine (0.13 g, 0.77 mmol), intermediate 79 (0.3 g, 0.92 mmol), Pd(OAc)₂ (4 mg, 0.02 mmol), PPh₃ (16 mg, 0.06 mmol) and NaHCO₃ (0.13 g, 1.55 mmol) in DMF (25 mL) was degassed with argon for 2 minutes and then refluxed at 160° C. for 2.5 h. After cooling to room temperature, the mixture was diluted with water (50 mL) to get a yellow precipitate. The solid was collected by filtration and washed by water. The crude was purified by silica gel column with CHCl₃ to 30% CH₃OH/CHCl₃ as eluents to yield a pale yellow solid (0.25 g, 78%).

¹H NMR (500 MHz, DMSO-d₆): δ 6.92 (s, 2H), 6.98 (d, J=16.4 Hz, 1H), 7.31 (d, J=16.4 Hz, 1H), 7.47 (d, J=8.8 Hz, 1H), 7.72 (dd, J=2.5 Hz, J=8.8 Hz, 1H), 7.81 (t, J=7.95 Hz, 1H), 7.99 (d, J=8.8 Hz, 1H), 8.19 (d, J=2.5 Hz, 1H), 8.29 (d, J=7.9 Hz, 1H), 8.33 (s, 1H), 8.56 (s, 2H), 10.62 (br s, 1H).

### Example 147

### N-[4-Chloro-3-(2-{2-[4-(Piperidine-4-sulfonyl)-phenylamino]-pyrimidin-5-yl}-vinyl)-phenyl]-3-trifluoromethyl-benzamide (Compound LXV)

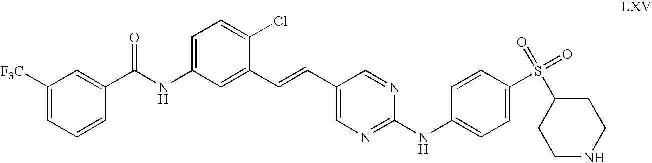

A mixture of intermediate 80 (0.16 g, 0.38 mmol), tert-butyl 4-(4-bromophenylsulfonyl)piperidine-1-carboxylate (0.19 g, 0.47 mmol), Pd₂(dba)₃ (35 mg, 0.04 mmol), Xantphos (44 mg, 0.08 mmol) and cesium carbonate (0.37 g, 1.13 mmol) were suspended in dioxane (30 mL) and degassed with argon for 2 minutes. The mixture was heated at reflux under the argon atmosphere for 2.5 h. After cooling down, the mixture was poured into water (50 mL) and extracted with CHCl₃ (3×50 mL). The organic layer was separated and washed with brine, dried over Na₂SO₄ and filtered. The filtrate was concentrated in vacuo and the crude product was purified by flash chromatography on silica gel with CHCl₃ to 10% MeOH/CHCl₃ as eluents to afford the product as a yellow solid (0.15 g, 53%).

The above product was dissolved in formic acid (5 mL) and stirred for overnight. The solution was basified to pH ca. 7 with 2M NaOH and extracted by CHCl₃ (3×50 mL). The organic layer was separated and washed with brine, dried over Na₂SO₄ and filtered. The filtrate was concentrated in vacuo and the crude product was purified by flash chromatography on silica gel with CHCl₃ to 30% MeOH/CHCl₃ as eluents to afford the product as an off-white solid (50 mg, 39%).

¹H NMR (500 MHz, DMSO-d₆): δ 1.43-1.55 (m, 2H), 1.81-1.92 (m, 2H), 2.50-2.62 (m, 2H), 3.00-3.22 (m, 2H), 7.13 (d, J=16.4 Hz, 1H), 7.45-7.55 (m, 2H), 7.72-7.83 (m, 3H), 7.82 (t, J=7.7 Hz, 1H), 7.99 (d, J=8.0 Hz, 1H), 8.07 (d, J=8.8 Hz, 1H), 8.22-8.34 (m, 3H), 8.92 (s, 2H), 10.50 (s, 1H), 10.71 (s, 1H).

### Example 148

### 5-Vinyl-1H-indole (Intermediate 81)

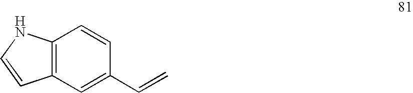

n-BuLi (1.6M in THF, 4.7 mL, 7.52 mmol) was added slowly to methyltriphenylphosphonium bromide (2.7 g, 7.56 mmol) in anhydrous THF (30 mL) at room temperature. After the solution was stirred at room temperature for 30 min, 1H-indole-5-carbaldehyde (0.55 g, 3.79 mmol) in THF (20 mL) was added into the solution and the mixture was refluxed for overnight. After cooling down, the reaction was quenched by methanol (2 mL) and the solvent was removed by rotovap. The crude product was purified by silica gel column with hexanes/CHCl₃ (v/v 1:1) as an eluent to yield a colorless oil (0.3 g, 55%).

¹H NMR (500 MHz, DMSO-d₆): δ 5.08 (dd, J=10.9, 0.9 Hz, 1H), 5.68 (dd, J=17.6, 1.0 Hz, 1H), 6.41 (t, J=2.2 Hz, 1H), 6.78 (dd, J=17.7, 10.9 Hz, 1H), 7.27 (dd, J=8.5, 1.5 Hz, 1H), 7.32-7.40 (m, 2H), 7.58 (s, 1H), 11.10 (br s, 1H).

### Example 149

### 5-((E)-2-(1H-indol-5-yl)vinyl)pyrimidin-2-amine (Intermediate 82)

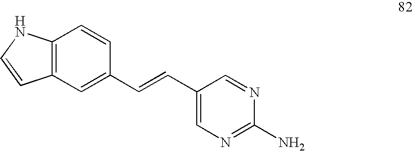

A mixture of 5-bromo-pyrimidin-2-amine (0.14 g, 0.80 mmol), intermediate 81 (16 g, 1.12 mmol), Pd(OAc)₂ (4 mg, 0.02 mmol), PPh₃ (17 mg, 0.06 mmol) and NaHCO₃ (0.13 g, 1.55 mmol) in DMF (10 mL) was degassed with argon for 2 minutes and then refluxed at 160° C. for 2.5 h. After cooling to room temperature, the mixture was diluted with water (20 mL) and extracted with CHCl₃ (2×70 mL). The organic layer was separated and washed with brine, dried over Na₂SO₄ and filtered. The filtrate was concentrated in vacuo and the crude product was purified by flash chromatography on silica gel with CHCl₃ to 10% MeOH/CHCl₃ as eluents to afford the intermediate as pale brown oil (0.12 g, 64%).

¹H NMR (500 MHz, DMSO-d₆): δ 6.42 (t, J=2.3 Hz, 1H), 6.69 (br s, 2H), 6.90 (d, J=16.6 Hz, 1H), 7.18 (d, J=16.5 Hz, 1H), 7.30-7.42 (m, 3H), 7.65 (s, 1H), 8.49 (s, 2H), 11.11 (s, 1H).

### Example 150

### {5-[2-(1H-Indol-5-yl)-vinyl]-pyrimidin-2-yl}-[4-(Piperidine-4-sulfonyl)-phenyl]-amine (Compound LXVI)

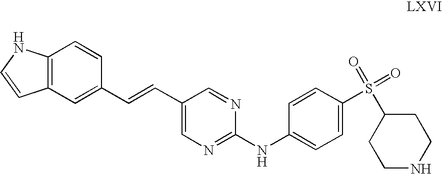

A solution of intermediate 5 (2.0 g, 4.95 mmol) and 2M HCl in dioxane (5 mL, 10 mmol) in CHCl₃ (50 mL) was stirred at room temperature for 30 min. The solid was collected by filtration and washed by CHCl₃ to yield 4-(4-bromophenylsulfonyl)piperidine hydrochloride as an off-white solid (1.1 g, 65%).

A mixture of intermediate 82 (0.10 g, 0.42 mmol), 4-(4-bromophenylsulfonyl)piperidine (0.14 g, 0.46 mmol), Pd₂(dba)₃ (39 mg, 0.04 mmol), Xantphos (49 mg, 0.08 mmol) and Cs₂CO₃ (0.41 g, 1.26 mmol) were suspended in dioxane (30 mL) and DMF (10 mL). The system was degassed with argon for 2 minutes and heated at reflux under the argon atmosphere for overnight. After cooling down, the solvent was removed by rotovap and the crude product was purified by flash chromatography on silica gel with CHCl₃ to 30% MeOH/CHCl₃ as eluents to afford the product as a yellow solid (50 mg, 53%).

¹H NMR (500 MHz, DMSO-d₆): δ 1.30-1.40 (m, 2H), 1.70-1.80 (m, 2H), 2.30-2.45 (m, 2H), 2.90-3.00 (m, 2H), 3.10-3.25 (m, 1H), 6.45 (t, J=2.2 Hz, 1H), 7.05 (d, J=16.5 Hz, 1H), 7.35 (t, J=2.7 Hz, 1H), 7.40 (t, J=9.7 Hz, 3H), 7.60-7.80 (m, 3H), 8.04 (d, J=8.9 Hz, 2H), 8.83 (s, 2H), 10.34 (s, 1H), 11.20 (s, 1H).

### Example 151

### 4-(2-{2-[4-(Piperidine-4-sulfonyl)-phenylamino]-pyrimidin-5-yl}-vinyl)-1,3-dihydro-indol-2-one hydrochloride (Compound LXVII)

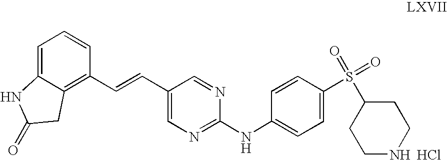

A mixture of intermediate 6 (50 mg, 0.11 mmol), 4-bromoindolin-2-one (36 mg, 0.17 mmol), Pd(OAc)₂ (5 mg, 0.02 mmol), PPh₃ (20 mg, 0.08 mmol) and NaHCO₃ (47 mg, 0.6 mmol) were suspended in DMF (15 mL). The system was degassed with argon for 2 minutes and heated at reflux under the argon atmosphere for 2 h.

After cooling down, the solvent was removed by rotovap and dissolved in CHCl₃ (10 mL) and 2M HCl in dioxane (3 mL). The mixture was refluxed for 4 h and concentrated under vacuum. The crude product was purified by flash chromatography on silica gel with 30% MeOH/CHCl₃ as eluents to afford the product as an orange solid (40 mg, 69% in 2-steps).

¹H NMR (500 MHz, DMSO-d₆): δ 1.70-1.80 (m, 2H), 1.90-2.05 (m, 2H), 2.80-2.90 (m, 2H), 3.20-3.30 (m, 2H), 3.66 (s, 2H), 6.76 (d, J=7.5 Hz, 1H), 7.15 (d, J=16.6 Hz, 1H), 7.20-7.30 (m, 3H), 7.74 (d, J=8.8 Hz, 1H), 8.09 (d, J=8.7 Hz, 1H), 8.90 (s, 2H), 9.11 (br s, 1H), 9.73 (br s, 1H), 10.50 (s, 1H), 10.54 (s, 1H).

### Example 152

### [3-(2-{2-[4-(Piperidine-4-sulfonyl)-phenylamino]-pyrimidin-5-yl}-vinyl)-phenyl]-methanol hydrochloride (Compound LXVIII)

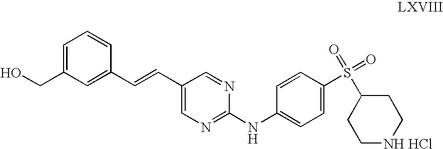

A mixture of intermediate 6 (60 mg, 0.14 mmol), (3-bromophenyl)methanol (50 mg, 0.27 mmol), Pd(OAc)₂ (5 mg, 0.02 mmol), PPh₃ (20 mg, 0.08 mmol) and NaHCO₃ (56 mg, 0.67 mmol) were suspended in DMF (20 mL). The system was degassed with argon for 2 minutes and heated at reflux under the argon atmosphere for 4.5 h. After cooling down, the solvent was removed by rotovap and purified by flash chromatography on silica gel with CHCl₃ to 10% MeOH/CHCl₃ as eluents to afford the product as a yellow solid.

The above product was dissolved in CHCl₃ (10 mL) and 2M HCl in dioxane (3 mL) and stirred at room temperature for 1 h. The precipitate was collected by filtration and washed by methanol to yield a yellow solid (50 mg, 76% in 2-steps). ¹H NMR (500 MHz, DMSO-d₆): δ 1.60-1.75 (m, 2H), 1.95-2.05 (m, 2H), 2.80-2.95 (m, 2H), 3.25-3.35 (m, 2H), 3.40-3.55 (m, 1H), 4.53 (s, 2H), 7.17 (d, J=16.7 Hz, 1H), 7.23 (d, J=7.6 Hz, 1H), 7.30-7.40 (m, 2H), 7.44 (d, J=7.8 Hz, 1H), 7.55 (s, 1H), 7.75 (d, J=8.9 Hz, 1H), 8.08 (d, J=8.8 Hz, 1H), 8.50 (br s, 1H), 8.87 (s, 2H), 9.05 (br s, 1H), 10.45 (s, 1H).

### Example 153

### 2-(4-Bromophenyl)-1H-imidazole (Intermediate 83)

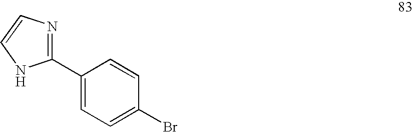

Glyoxal solution (40 wt. % in water, 4 mL) and conc. NH₄OH (5.7 ml) were added into 4-bromobenzaldehyde (3 g, 16.22 mmol) in EtOH (45 mL) at 0° C. and stirred at room temperature for 30 min. The mixture was then refluxed for 4.5 h. The solvent was removed by rotovap and the precipitate was collected by filtration, washed by CHCl₃ to yield a yellow solid (1.5 g, 41%). ¹H NMR (500 MHz, DMSO-d₆): δ 7.04 (br s, 1H), 7.26 (br s, 1H), 7.64 (d, J=8.5 Hz, 2H), 7.87 (d, J=8.6 Hz, 2H), 12.58 (br s, 1H).

### Example 154

### (5-{2-[4-(1H-Imidazol-2-yl)-phenyl]-vinyl}-pyrimidin-2-yl)-[4-(piperidine-4-sulfonyl)-phenyl]-amine hydrochloride (Compound LXIX)

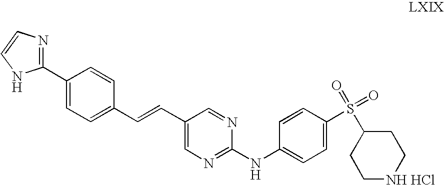

A mixture of intermediate 6 (0.13 g, 0.28 mmol) intermediate 22 (0.1 g, 0.42 mmol), Pd(OAc)₂ (5 mg, 0.02 mmol), PPh₃ (10 mg, 0.04 mmol) and Et₃N (1 mL, 7.2 mmol) were suspended in DMF (20 mL). The system was degassed with argon for 2 minutes and heated at reflux under the argon atmosphere in a sealed tube for 5 h. After cooling to room temperature, the mixture was diluted with water (20 mL) and extracted with CHCl₃ (3×50 mL). The organic layer was separated and washed with brine, dried over Na₂SO₄ and filtered. The filtrate was concentrated in vacuo and the crude product was purified by flash chromatography on silica gel with CHCl₃ to 10% MeOH/CHCl₃ as eluents to afford the product as pale yellow solid.

The above product was dissolved in CHCl₃ (20 mL) and 2M HCl in dioxane (5 mL) and stirred at room temperature for 1 h. The precipitate was collected by filtration and washed by acetone to yield an orange solid (50 mg, 34% in 2-steps). ¹H NMR (500 MHz, DMSO-d₆): δ 1.60-1.80 (m, 2H), 1.90-2.10 (m, 2H), 2.80-2.95 (m, 2H), 3.20-3.40 (m, 2H), 7.30-7.35 (m, 2H), 7.43 (d, J=3.6 Hz, 2H), 7.76 (d, J=9.0 Hz, 2H), 7.81 (s, 2H), 7.83 (d, J=8.6 Hz, 2H), 8.09 (d, J=8.9 Hz, 2H), 8.20 (d, J=8.6 Hz, 2H), 8.55-8.65 (m, 1H), 8.90 (s, 2H), 9.10-9.15 (m, 1H), 10.53 (s, 1H), 15.12 (br s, 1H).

### Example 155

### (5-{2-[3-(1H-Imidazol-2-yl)-phenyl]-vinyl}-pyrimidin-2-yl)-[4-(piperidine-4-sulfonyl)-phenyl]-amine (Compound LXX)

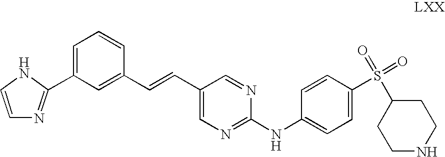

Glyoxal solution (40 wt. % in water, 4 mL) and conc. NH₄OH (5.7 ml) were added into 4-bromobenzaldehyde (3.6 g, 19.46 mmol) in EtOH (30 mL) at 0° C. and stirred at room temperature for 30 min. The mixture was then refluxed for 3 h. The solvent was removed by rotovap and purified by silica gel column with 5% CH₃OH/CHCl₃ as an eluent to yield 2-(3-bromophenyl)-1H-imidazole as a dark yellow solid after washed by CHCl₃ (1.9 g, 44%).

A mixture of intermediate 6 (0.25 g, 0.56 mmol), 2-(3-bromophenyl)-1H-imidazole (0.19 g, 0.85 mmol), Pd(OAc)₂ (5 mg, 0.02 mmol), PPh₃ (10 mg, 0.04 mmol) and Et₃N (1 mL, 7.2 mmol) were suspended in DMF (20 mL). The system was degassed with argon for 2 minutes and heated at reflux under the argon atmosphere in a sealed tube for 5 h. After cooling to room temperature, the mixture was diluted with water (20 mL) and extracted with CHCl₃ (3×50 mL). The organic layer was separated and washed with brine, dried over Na₂SO₄ and filtered. The filtrate was concentrated in vacuo and the crude product was purified by flash chromatography on silica gel with CHCl₃ to 15% CH₃OH/CHCl₃ as eluents to afford the product as pale yellow solid.

The above product was dissolved in CHCl₃ (20 mL) and 2M HCl in dioxane (5 mL) and stirred at room temperature for 1 h. The precipitate was collected by filtration. The solid was dissolved in CHCl₃ (50 mL) and washed by saturated NaHCO₃ (2×25 mL). The organic layer was separated and washed with brine, dried over Na₂SO₄ and filtered. The filtrate was concentrated in vacuo to yield a pale yellow solid after washed by acetone (0.15 g, 55%).

¹H NMR (500 MHz, DMSO-d₆): δ 1.25-1.40 (m, 2H), 1.70-1.80 (m, 2H), 2.30-2.45 (m, 2H), 2.90-3.00 (m, 2H), 3.10-3.25 (m, 1H), 7.05 (s, 1H), 7.24 (d, J=16.6 Hz, 1H), 7.28 (s, 1H), 7.40 (d, J=16.7 Hz, 1H), 7.46 (t, J=8.7 Hz, 1H), 7.53 (d, J=7.6 Hz, 1H), 7.73 (d, J=8.7 Hz, 2H), 7.82 (d, J=8.7 Hz, 1H), 8.05 (d, J=8.8 Hz, 2H), 8.21 (s, 1H), 8.89 (s, 2H), 10.40 (s, 1H), 12.57 (br s, 1H).

### Example 156

### [4-(Piperidine-4-sulfonyl)-phenyl]-(5-{2-[4-(1H-tetrazol-5-yl)-phenyl]-vinyl}-pyrimidin-2-yl)-amine hydrochloride (Compound LXXI)

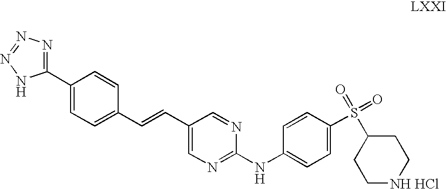

A mixture of intermediate 6 (0.25 g, 0.56 mmol), 5-(4-bromophenyl)-1H-tetrazole (0.19 g, 0.85 mmol), Pd(OAc)₂ (5 mg, 0.02 mmol), PPh₃ (10 mg, 0.04 mmol) and Et₃N (1 mL, 7.2 mmol) were suspended in DMF (20 mL). The system was degassed with argon for 2 minutes and heated at reflux under the argon atmosphere in a sealed tube for 5 h. After cooling to room temperature, the mixture was diluted with water (20 mL) and extracted with CHCl₃ (3×50 mL). The organic layer was separated and washed with brine, dried over Na₂SO₄ and filtered. The filtrate was concentrated in vacuo and the crude product was purified by flash chromatography on silica gel with CHCl₃ to 15% MeOH/CHCl₃ as eluents to afford the product as pale yellow solid.

The above product was dissolved in CHCl₃ (20 mL) and 2M HCl in dioxane (5 mL) and stirred at room temperature for 1 h. The precipitate was collected by filtration. The solid was dissolved in CHCl₃ (50 mL) and washed by sat. NaHCO₃ (2×25 mL). The organic layer was separated and washed with brine, dried over Na₂SO₄ and filtered. The filtrate was concentrated in vacuo to yield a pale yellow solid after washed by acetone (0.15 g, 55%).

¹H NMR (500 MHz, DMSO-d₆): δ 1.60-1.80 (m, 2H), 1.95-2.05 (m, 2H), 2.30-2.45 (m, 2H), 2.80-2.95 (m, 2H), 3.25-3.35 (m, 2H), 3.45-3.55 (m, 1H), 7.36 (d, J=16.6 Hz, 1H), 7.44 (d, J=16.6 Hz, 1H), 7.70-7.85 (m, 4H), 8.10 (t, J=8.6 Hz, 1H), 8.50-8.65 (m, 1H), 8.90 (s, 2H), 9.10-9.20 (m, 1H), 10.51 (s, 1H). MS(ES+): m/z 489 (M+H)⁺.

### Example 157

### [4-(Piperidine-4-sulfonyl)-phenyl]-(5-{2-[3-(1H-tetrazol-5-yl)-phenyl]-vinyl}-pyrimidin-2-yl)-amine (Compound LXXII)

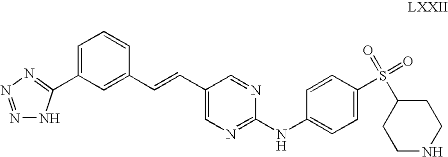

A mixture of intermediate 6 (0.20 g, 0.45 mmol), 5-(3-bromophenyl)-1H-tetrazole (0.15 g, 0.67 mmol), Pd(OAc)₂ (5 mg, 0.02 mmol), PPh₃ (10 mg, 0.04 mmol) and Et₃N (0.5 mL, 3.6 mmol) were suspended in DMF (20 mL). The system was degassed with argon for 2 minutes and heated at reflux under the argon atmosphere in a sealed tube for 8 h. After cooling to room temperature, the mixture was diluted with water (20 mL) and extracted with CHCl₃ (3×50 mL). The organic layer was separated and washed with brine, dried over Na₂SO₄ and filtered. The filtrate was concentrated in vacuo and the crude product was purified by flash chromatography on silica gel with CHCl₃ to 30% MeOH/CHCl₃ as eluents to afford the product as pale yellow solid.

The above product was dissolved in CHCl₃ (20 mL) and 2M HCl in dioxane (5 mL) and stirred at room temperature for 1 h. The precipitate was collected by filtration. The solid was dissolved in CHCl₃ (50 mL) and washed by saturated NaHCO₃ (2×25 mL). The organic layer was separated and washed with brine, dried over Na₂SO₄ and filtered. The filtrate was concentrated in vacuo to yield a pale yellow solid (0.12 g, 54%).

¹H NMR (500 MHz, DMSO-d₆): δ 1.55-1.75 (m, 2H), 1.95-2.05 (m, 2H), 2.80-2.95 (m, 2H), 7.22 (d, J=16.6 Hz, 1H), 7.35-7.55 (m, 3H), 7.75 (d, J=8.6 Hz, 2H), 7.89 (d, J=7.6 Hz, 1H), 8.08 (d, J=8.7 Hz, 2H), 8.23 (s, 1H), 8.91 (s, 2H), 10.45 (s, 1H).

### Example 158

### 4-(4-Bromophenylsulfonyl)-1-methylpiperidine (Intermediate 84)

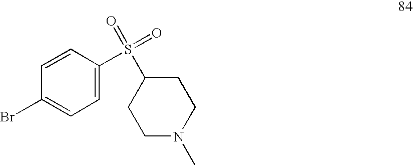

A solution of 4-(4-bromophenylsulfonyl)piperidine (0.89 g, 2.94 mmol), CH₃I (0.44 g, 3.10 mmol), Et₃N (1.02 mL, 7.35 mmol) in CHCl₃ (30 mL) was stirred at room temperature for 6 h. The solvent was removed by rotovap and the crude product was purified by flash chromatography on silica gel with 5% MeOH/CHCl₃ as an eluent to afford the intermediate as a white solid after washed by methanol (0.55 g, 59%). ¹H NMR (500 MHz, DMSO-d₆): δ 1.65-1.80 (m, 2H), 2.07 (d, J=11.7 Hz, 2H), 2.73 (s, 3H), 2.80-3.00 (m, 2H), 3.47 (d, J=9.8 Hz, 2H), 3.57 (t, J=11.8 Hz, 1H), 7.79 (d, J=8.5 Hz, 2H), 7.94 (d, J=8.4 Hz, 2H), 9.2 (br s, 1H).

### Example 159

### N-(4-(1-Methylpiperidin-4-ylsulfonyl)phenyl)-5-vinylpyrimidin-2-amine (Compound LXXIII)

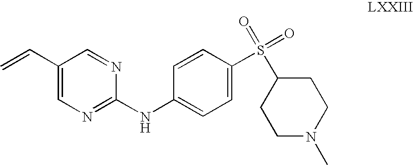

A mixture of intermediate 1 (76 mg, 0.62 mmol), intermediate 84 (0.20 g, 0.63 mmol), Pd₂(dba)₃ (58 mg, 0.06 mmol), Xantphos (65 mg, 0.13 mmol) and Cs₂CO₃ (0.61 g, 1.88 mmol) were suspended in DMF (30 mL). The system was degassed with argon for 2 minutes and heated at reflux under the argon atmosphere for overnight. After cooling down, the solvent was removed by rotovap and the crude product was purified by flash chromatography on silica gel with 10% MeOH/CHCl₃ as an eluent to afford the product as a pale yellow solid after washed by methanol (0.15 g, 67%).

¹H NMR (500 MHz, DMSO-d₆): δ 1.40-1.55 (m, 2H), 1.70-1.85 (m, 4H), 2.10 (s, 3H), 2.70-2.85 (m, 2H), 2.95-3.05 (m, 1H), 5.29 (d, J=11.1 Hz, 1H), 5.92 (d, J=18.0 Hz, 1H), 6.60-6.70 (m, 1H), 7.73 (d, J=8.6 Hz, 2H), 8.03 (d, J=8.7 Hz, 2H), 8.73 (s, 2H), 10.36 (s, 1H).

### Example 160

### N-(4-(1-methylpiperidin-4-ylsulfonyl)phenyl)-5-((E)-2-(1H-indazol-4-yl)vinyl)pyrimidin-2-amine (LXXIV)

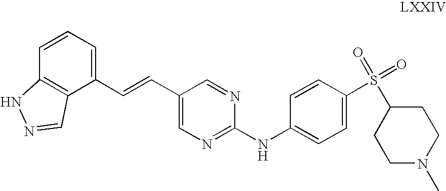

A mixture of the above-described compound LXXIII (0.10 g, 0.28 mmol), 4-bromo-1H-indazole (80 mg, 0.41 mmol), Pd(OAc)₂ (5 mg, 0.02 mmol), PPh₃ (10 mg, 0.04 mmol) and Et₃N (0.8 mL, 5.8 mmol) were suspended in DMF (20 mL). The system was degassed with argon for 2 minutes and heated at reflux under the argon atmosphere in a sealed tube for 4 h. After cooling to room temperature, the solution was concentrated in vacuo and the crude product was purified by flash chromatography on silica gel with CHCl₃ to 5% MeOH/CHCl₃ as eluents to afford the product as off-white solid after washed by methanol (77 mg, 58%).

¹H NMR (500 MHz, DMSO-d₆): δ 1.45-1.55 (m, 2H), 1.70-1.85 (m, 4H), 2.10 (s, 3H), 2.70-2.85 (m, 2H), 3.00-3.15 (m, 1H), 7.19 (d, J=16.5 Hz, 1H), 7.29 (t, J=8.6 Hz, 1H), 7.35 (d, J=16.6 Hz, 1H), 7.40 (t, J=8.6 Hz, 2H), 7.58 (d, J=8.6 Hz, 2H), 7.74 (d, J=8.5 Hz, 2H), 8.85 (s, 2H), 10.4 (s, 1H).

### Example 161

### 4-{5-[2-(1H-Indazol-4-yl)-vinyl]-pyrimidin-2-ylamino}-N-piperidin-4-yl-benzenesulfonamide hydrochloride (Compound LXXV)

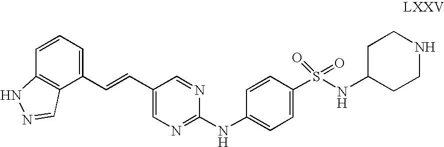

A mixture of intermediate 60 (0.15 g, 0.33 mmol), 4-bromo-1H-indazole (80 mg, 0.41 mmol), Pd(OAc)₂ (10 mg, 0.04 mmol), PPh₃ (20 mg, 0.08 mmol) and Et₃N (1 mL, 7.2 mmol) were suspended in DMF (25 mL). The system was degassed with argon for 2 min. and heated at reflux under the argon atmosphere in a sealed tube for 5 h. After cooling to room temperature, the mixture was diluted with water (20 mL) and extracted with CHCl₃ (3×50 mL). The organic layer was separated and washed with brine, dried over Na₂SO₄ and filtered. The filtrate was concentrated in vacuo and the crude product was purified by flash chromatography on silica gel with CHCl₃ to 15% MeOH/CHCl₃ as eluents to afford the product as pale yellow solid.

The above product was dissolved in CHCl₃ (20 mL) and 2M HCl in dioxane (5 mL) and stirred at room temperature for 1 h. The precipitate was collected by filtration. The solid was dissolved in CHCl₃ (50 mL) and washed by sat. NaHCO₃ (2×25 mL). The organic layer was separated and washed with brine, dried over Na₂SO₄ and filtered. The filtrate was concentrated in vacuo to yield a pale yellow solid after washed by acetone (0.15 g, 55%).

¹H NMR (500 MHz, DMSO-d₆): δ 1.50-1.65 (m, 2H), 1.70-1.80 (m, 2H), 2.80-2.95 (m, 2H), 3.10-3.20 (m, 2H), 3.20-3.30 (m, 1H), 7.30-7.35 (m, 2H), 7.42 (d, J=16.7 Hz, 1H), 7.47 (d, J=8.6 Hz, 1H), 7.70 (d, J=16.6 Hz, 1H), 7.76 (d, J=8.6 Hz, 2H), 7.81 (d, J=8.6 Hz, 1H), 8.00 (d, J=8.6 Hz, 2H), 8.58 (br s, 1H), 8.60 (s, 1H), 8.74 (br s 1H), 8.98 (s, 2H), 10.34 (s, 1H).

### Example 162

### 4-(4-{5-[2-(1H-Indazol-4-yl)-vinyl]-pyrimidin-2-ylamino}-benzenesulfonyl)-[1,4]diazepane-1-carboxylic acid tert-butyl ester (Intermediate 85)

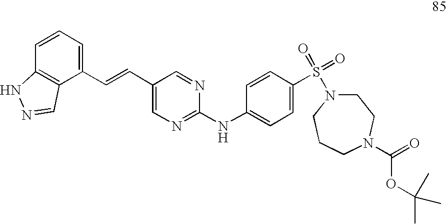

A mixture of intermediate 1 (0.19 g, 1.57 mmol), intermediate 30 (0.6 g, 1.43 mmol), Pd₂(dba)₃ (50 mg, 0.05 mmol), Xantphos (58 mg, 0.1 mmol) and cesium carbonate (1.5 g, 4.6 mmol) were suspended in DMF (20 mL). The system was degassed with argon for 2 minutes and heated at reflux under the argon atmosphere for 5 h. After cooling down, the solvent was removed by rotovap and the crude product was purified by silica gel column with 5% CH₃OH/CHCl₃ as an eluent to yield 4-[4-(5-vinyl-pyrimidin-2-ylamino)-benzenesulfonyl]-[1,4]diazepane-1-carboxylic acid tert-butyl ester as pale yellow solid after washed by methanol (0.35 g, 43%).

A mixture of 4-[4-(5-vinyl-pyrimidin-2-ylamino)-benzenesulfonyl]-[1,4]diazepane-1-carboxylic acid tert-butyl ester (0.15 g, 0.33 mmol), 4-bromo-1H-indazole (80 mg, 0.41 mmol), Pd(OAc)₂ (10 mg, 0.04 mol), PPh₃ (20 mg, 0.08 mmol) and Et₃N (1 mL, 7.2 mmol) were suspended in DMF (15 mL). The system was degassed with argon for 2 min. and heated at reflux under the argon atmosphere in a sealed tube for 3 h. After cooling to room temperature, the mixture was diluted with water (20 mL) and extracted with CHCl₃ (3×50 mL). The organic layer was separated and washed with brine, dried over Na₂SO₄ and filtered. The filtrate was concentrated in vacuo and the crude product was purified by flash chromatography on silica gel with CHCl₃ to 5% MeOH/CHCl₃ as eluents to afford the intermediate as an off-white solid after washed by CH₃OH (0.14 g, 74%).

¹H NMR (500 MHz, DMSO-d₆): δ 1.37 (d, J=3.9 Hz, 9H), 1.60-1.75 (m, 2H), 3.10-3.20 (m, 2H), 3.20-3.30 (m, 2H), 3.40-3.50 (m, 2H), 7.30-7.40 (m, 2H), 7.41 (d, J=16.7 Hz, 1H), 7.47 (d, J=6.7 Hz, 1H), 7.65-7.75 (m, 3H), 8.02 (d, J=8.9 Hz, 2H), 8.60 (s, 1H), 8.98 (s, 2H), 10.35 (s, 1H), 13.18 (br s, 1H).

### Example 163

### [4-([1,4]Diazepane-1-sulfonyl)-phenyl]-{5-[2-(1H-indazol-4-yl)-vinyl]-pyrimidin-2-yl}-amine (Compound LXXVI)

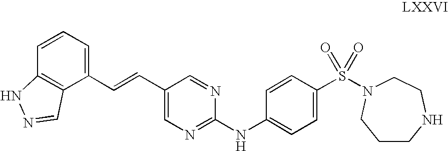

2M HCl in dioxane (5 mL) was added to intermediate 85 (0.1 g, 0.17 mmol) in CHCl₃ (10 mL) and CH₃OH (10 mL). The mixture was stirred at room temperature for 2 h. The solvent was removed by rotovap and the solid was suspended in saturated NaHCO₃ (50 mL) and extracted by EtOAc (3×25 mL). The organic layer was separated and washed with brine, dried over Na₂SO₄ and filtered. The filtrate was concentrated in vacuo to yield a pale yellow solid after washed by acetone (50 mg, 60%).

¹H NMR (500 MHz, DMSO-d₆): δ 1.60-1.75 (m, 2H), 2.65-2.75 (m, 2H), 3.05-3.20 (m, 4H), 7.30-7.35 (m, 2H), 7.41 (d, J=16.7 Hz, 1H), 7.47 (d, J=8.6 Hz, 1H), 7.60-7.75 (m, 3H), 7.95-8.05 (m, 2H), 8.60 (s, 1H), 8.97 (s, 2H), 10.32 (br s, 1H), 13.25 (br s, 1H).

### Example 164

### 4-[4-(5-Vinyl-pyrimidin-2-ylamino)-benzoyl]-piperazine-1-carboxylic acid tert-butyl ester (Intermediate 86)

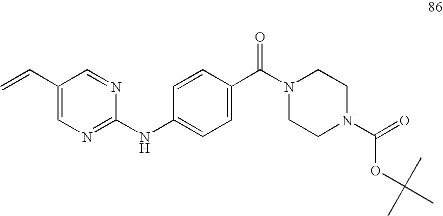

To a stirring solution of piperazine-1-carboxylic acid tert-butyl ester (1.10 g, 5.91 mmol) and Et₃N (3 mL, 21.6 mmol) in toluene (30 mL), 4-bromobenzoyl chloride (1.02 g, 4.68 mmol) was added dropwised at room temperature. The mixture was stirred at room temperature under argon for overnight. The solvent was removed under reduced pressure and purified by silica gel column with CHCl₃ to 5% CH₃OH/CHCl₃ as eluents to afford 4-(4-bromo-benzoyl)-piperazine-1-carboxylic acid tert-butyl ester as a pale yellow solid (1.2 g, 69%).

A mixture of intermediate 1 (0.13 g, 1.08 mmol), 4-(4-bromo-benzoyl)-piperazine-1-carboxylic acid tert-butyl ester (0.40 g, 1.08 mmol), Pd₂(dba)₃ (10 mg, 0.01 mmol), Xantphos (20 mg, 0.02 mmol) and Cs₂CO₃ (1.06 g, 3.25 mmol) were suspended in dioxane (50 mL). The system was degassed with argon for 2 minutes and heated at reflux under the argon atmosphere for 6 h. The solvent was removed by rotovap and the solid was suspended in sat. NaHCO₃ (50 mL) and extracted by CHCl₃ (3×25 mL). The organic layer was separated and washed with brine, dried over Na₂SO₄ and filtered. The filtrate was concentrated in vacuo and purified by silica gel column with CHCl₃ to 3% CH₃OH/CHCl₃ as eluents to yield the intermediate as an off-white solid after washed by methanol (0.25 g, 56%).

¹H NMR (500 MHz, DMSO-d₆): δ 1.41 (s, 9H), 3.37 (br s, 4H), 3.47 (br s, 4H), 5.25 (d, J=11.3 Hz, 1H), 5.88 (d, J=17.8 Hz, 1H), 6.64 (dd, J=17.8, 11.2 Hz, 1H), 7.37 (d, J=8.6 Hz), 7.84 (d, J=8.6 Hz), 8.67 (s, 2H), 10.02 (s, 1H).

### Example 165

### (4-{5-[2-(1H-Indazol-4-yl)-vinyl]-pyrimidin-2-ylamino}-phenyl)-piperazin-1-yl-methanone hydrochloride (Compound LXXVII)

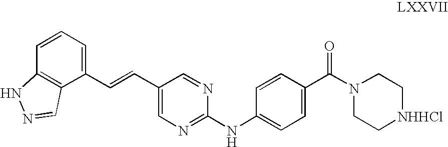

A mixture of intermediate 86 (0.15 g, 0.33 mmol), 4-bromo-1H-indazole (90 mg, 0.46 mmol), Pd(OAc)₂ (10 mg, 0.04 mmol), PPh₃ (20 mg, 0.08 mmol) and Et₃N (1 mL, 7.2 mmol) were suspended in DMF (10 mL). The system was degassed with argon for 2 minutes and heated at reflux under the argon atmosphere in a sealed tube for 5 h. After cooling to room temperature, the mixture was diluted with water (20 mL) and extracted with CHCl₃ (3×50 mL). The organic layer was separated and washed with brine, dried over Na₂SO₄ and filtered. The filtrate was concentrated in vacuo and the crude product was purified by flash chromatography on silica gel with CHCl₃ to 15% MeOH/CHCl₃ as eluents to afford the product as pale yellow solid.

The above product was dissolved in CHCl₃ (20 mL) and 2M HCl in dioxane (5 mL) and stirred at reflux for 20 min. The precipitate was collected by filtration and washed by CHCl₃ to yield the product as a yellow solid (0.15 g, 55%).

¹H NMR (500 MHz, DMSO-d₆): δ 3.25 (br s, 4H), 3.74 (br s, 4H), 7.30-7.40 (m, 2H), 7.40-7.50 (m, 2H), 7.68 (d, J=16.7 Hz, 1H), 7.89 (d, J=8.7 Hz, 2H), 8.60 (s, 1H), 8.94 (s, 2H), 9.36 (br s, 2H), 10.13 (s, 1H).

### Example 166

### N-(4-(2-(Pyrrolidin-1-yl)ethoxy)phenyl)-5-(2-chloro-5-methoxystyryl)pyrimidin-2-amine (Intermediate 87)

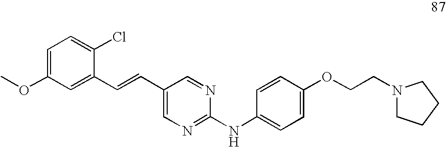

A mixture of intermediate 70 (185 mg, 0.71 mmol), 1-(2-(4-bromophenoxy)ethyl)pyrrolidine (181 mg, 0.88 mmol), Pd(OAc)₂ (17.5 mg, 0.08 mmol), xantphos (87 mg, 0.15 mmol) and ^{t}BuOK (163 mg, 1.45 mmol) in dioxane (4 mL) was irradiated in the microwave at 160° C. for 15 minutes. The reaction was filtered and the solids were washed with water, taken up in excess DCM, and purified by gradient flash chromatography (0-20% MeOH in DCM). The resulting fractions were concentrated in vacuo to afford the title intermediate as a white solid (57 mg, 18%). MS (ES+): m/z 451 (M+H)⁺.

### Example 167

### 3-((E)-2-(2-(4-(2-(Pyrrolidin-1-yl)ethoxy)phenylamino)pyrimidin-5-yl)vinyl)-4-chlorophenol (Compound LXXVIII)

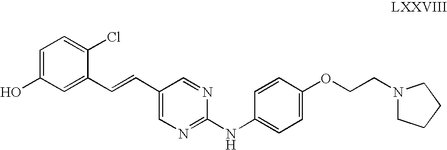

To a solution of intermediate 87 (57 mg, 0.13 mmol) in DCM (4 mL) was added BBr₃ (120 μL, 1.27 mmol) and the reaction was stirred for 3 hours. MeOH was added to quench the reaction and the mixture was concentrated in vacuo. The crude material was purified by preparative HPLC, and conc. NaHCO₃ was added to the resulting fractions until a pH of 8 was attained. The basic aqueous fractions were extracted with EtOAc (2×50 mL) and the combined organic layers were concentrated in vacuo. The residue was taken up in MeOH and 2 drops of conc. HCl were added. The mixture was concentrated in vacuo, precipitated from MeOH/Et₂O, and filtered to obtain the HCl salt of The title compound as a yellow solid (24 mg, 43%).

¹H NMR (500 MHz, DMSO-d₆): δ 1.86-1.94 (m, 2H), 1.97-2.07 (m, 2H), 3.07-3.14 (m, 2H), 3.53-3.62 (m, 4H), 4.31 (t, J=5.1 Hz, 2H), 6.74 (dd, J=8.7, 2.9 Hz, 1H), 6.98 (d, J=9.1 Hz, 2H), 7.06 (d, J=16.4 Hz, 1H), 7.17 (d, J=2.9 Hz, 1H), 7.25 (d, J=8.6 Hz, 1H), 7.31 (d, J=16.4 Hz, 1H), 7.69 (d, J=9.2 Hz, 2H), 8.73 (s, 2H), 9.76 (s, 1H), 10.60 (br s, 1H). MS (ES+): m/z 437 (M+H)⁺.

### Example 168

### N-(4-(2-(Pyrrolidin-1-yl)ethoxy)phenyl)-5-(3-methoxystyryl)pyrimidin-2-amine (Intermediate 88)

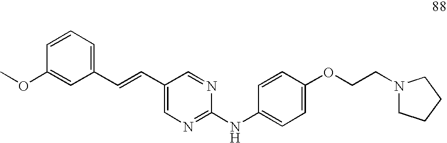

A mixture of intermediate 13 (114 mg, 0.50 mmol), 1-(2-(4-bromophenoxy)ethyl)pyrrolidine (117 mg, 0.57 mmol), Pd(OAc)₂ (10.5 mg, 0.05 mmol), xantphos (56 mg, 0.10 mmol) and ^{t}BuOK (108 mg, 0.96 mmol) in dioxane (2.5 mL) was irradiated in the microwave at 160° C. for 15 minutes. The reaction was filtered, the filtrate concentrated in vacuo, and the resulting crude material purified by gradient flash chromatography (0-20% MeOH in DCM) to afford the title intermediate as a white solid (44 mg, 21%). MS (ES+): m/z 417 (M+H)⁺.

### Example 169

### 3-((E)-2-(2-(4-(2-(Pyrrolidin-1-yl)ethoxy)phenylamino)pyrimidin-5-yl)vinyl)phenol (Compound LXXIX)

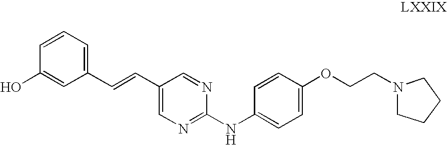

To a solution of intermediate 88 (44 mg, 0.11 mmol) in DCM (2 mL) was added BBr₃ (99 μL, 1.05 mmol) and the reaction was stirred for 40 minutes. MeOH was added to quench the reaction and the mixture was concentrated in vacuo. The crude material was purified by preparative HPLC and the resulting fractions were concentrated in vacuo to obtain the TFA salt of The title compound as a pale-yellow solid (14 mg, 33%).

¹H NMR (500 MHz, DMSO-d₆): δ 1.84-1.93 (m, 2H), 1.97-2.07 (m, 2H), 3.07-3.18 (m, 2H), 3.53-3.63 (m, 4H), 4.26 (t, J=5.0 Hz, 2H), 6.67 (dd, J=7.8, 2.2 Hz, 1H), 6.93 (s, 1H), 6.95-6.99 (m, 2H), 7.02 (d, J=16.6 Hz, 1H), 7.12-7.19 (m, 2H), 7.69 (d, J=9.1 Hz, 2H), 8.70 (s, 2H), 9.44 (s, 1H), 9.67 (s, 1H), 9.73 (br s, 1H). MS (ES+): 403 m/z (M+H)⁺.

### Example 170

### 4-Vinyl-1H-indole (Intermediate 89)

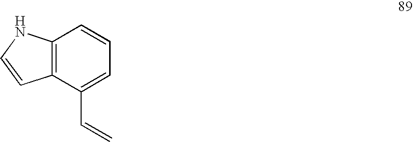

A mixture of 4-bromo-1H-indole (384 μL, 3.06 mmol), 4,4,5,5-tetramethyl-2-vinyl-1,3,2-dioxaborolane (2.05 g, 13.31 mmol), Pd(PPh₃)₄ (350 mg, 0.30 mmol), 2M Na₂CO₃ (6 mL, 12 mmol) in DME/EtOH (4:1, 30 mL) was heated to 95° C. for 18 h. The reaction was filtered, and the filtrate was concentrated in vacuo. The crude material was purified by gradient flash chromatography (0-100% EtOAc/hexanes) to afford the title intermediate as a clear oil that was used directly in the next reaction (270 mg, 62%).

### Example 171

### 5-((E)-2-(1H-Indol-4-yl)vinyl)pyrimidin-2-amine (Intermediate 90)

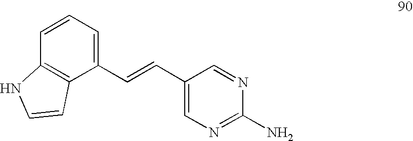

A mixture of intermediate 89 (270 mg, 1.89 mmol), 5-bromopyrimidin-2-amine (279 mg, 1.60 mmol), Pd(OAc)₂ (7.7 mg, 0.03 mmol), PPh₃ (36 mg, 0.14 mmol) and NaHCO₃ (280 mg, 3.33 mmol) in DMF (13 mL) was purged with argon and heated at 160° C. for 2 h. The reaction mixture was filtered and the solids rinsed with DCM and MeOH. The filtrate was concentrated in vacuo. The crude material was purified using gradient flash chromatography (0-100% EtOAc/hexanes), the resulting fractions were concentrated in vacuo, and triturated with Et₂O to afford the title intermediate as a yellow solid (128 mg, 54%). This intermediate was also synthesized via a different route. See intermediate 11.

¹H NMR (500 MHz, DMSO-d₆): δ 6.75 (s, 2H), 6.88 (br s, 1H), 7.08 (t, J=7.8 Hz, 1H), 7.12 (d, J=16.7 Hz, 1H), 7.25 (d, J=7.3 Hz, 1H), 7.30 (d, J=8.0 Hz, 1H), 7.39 (t, J=2.8 Hz, 1H), 7.47 (d, J=16.7 Hz, 1H). MS (ES+): m/z 237 (M+H)⁺.

### Example 172

### tert-Butyl 4-(4-(5-((E)-2-(1H-indol-4-yl)vinyl)pyrimidin-2-ylamino)phenylsulfonyl)piperidine-1-carboxylate (Intermediate 91)

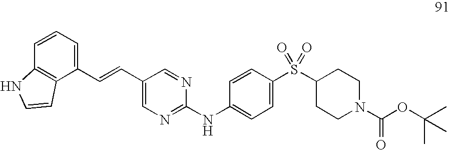

A mixture of intermediate 90 (75 mg, 0.32 mmol), intermediate 5 (131 mg, 0.32 mmol), Pd(OAc)₂ (3.5 mg, 0.02 mmol), xantphos (20 mg, 0.03 mmol) and ^{t}BuOK (78 mg, 0.70 mmol) in dioxane (3.5 mL) and DMF (0.25 mL) was irradiated in the microwave at 160° C. for 15 minutes. The reaction was filtered, the filtrate concentrated in vacuo, and the resulting crude material purified by gradient flash chromatography (0-100% EtOAC/hexanes) to afford the title intermediate as a white solid (40 mg, 22%). MS (ES+): m/z 560 (M+H)⁺.

### Example 173

### 5-((E)-2-(1H-Indol-4-yl)vinyl)-N-(4-(piperidin-4-ylsulfonyl)phenyl)pyrimidin-2-amine (Compound LXXX)

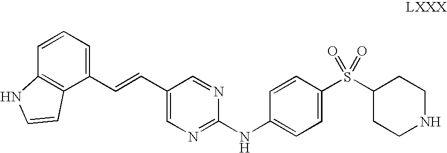

To a solution of intermediate 91 (17.4 mg, 0.03 mmol) in DCM (13.5 mL) was added TFA (416 μL, 5.40 mmol). The reaction was monitored by LCMS which showed no more intermediate 20 after 2.5 hours. The reaction was diluted with DCM (15 mL) and water (20 mL). The aqueous layer was brought to a basic pH using 30% NaOH (2-5 mL) and the layers were separated. The aqueous layer was extracted with DCM (20 mL) and EtOAC (20 mL). The combined organic layers were dried (Na₂SO₄), filtered, and concentrated in vacuo to afford the title compound as a tan solid (5 mg, 35%).

¹H NMR (500 MHz, DMSO-d₆): δ 1.27-1.38 (m, 2H), 1.68-1.76 (m, 2H), 2.35-2.45 (m, 2H), 2.90-3.00 (m, 2H), 3.17-3.12 (m, 1H), 6.94 (s, 1H), 7.12 (t, J=7.7 Hz, 1H), 7.26 (d, J=16.7 Hz, 1H), 7.32 (d, J=7.3 Hz, 1H), 7.35 (d, J=8.2 Hz, 1H), 7.44 (t, J=2.8 Hz, 1H), 7.69 (d, J=16.7 Hz, 1H), 7.73 (d, J=8.8 Hz, 2H), 8.06 (d, J=8.8 Hz, 2H), 8.95 (s, 2H), 10.38 (s, 1H), 11.21 (s, 1H). MS (ES+): m/z 460 (M+H)⁺.

### Example 174

### tert-Butyl 4-(4-(5-((E)-2-(1H-indazol-4-yl)vinyl)pyrimidin-2-ylamino)phenylsulfonyl)piperidine-1-carboxylate (Intermediate 92)

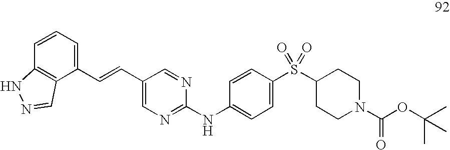

A mixture of 4-bromo-1H-indazole (160 mg, 0.81 mmol), 6 (302 mg, 0.68 mmol), Pd(OAc)₂ (9.3 mg, 0.04 mmol), PPh₃ (27 mg, 0.10 mmol), TEA (0.56 mL, 4.01 mmol) in DMF (4 mL) was irradiated in the microwave at 180° C. for 20 min. The reaction was filtered and the filtrate concentrated in vacuo. The crude material was purified by gradient flash chromatography (0-100% EtOAc/hexanes) to afford the title intermediate as a pale-yellow solid (127 mg, 33%). MS (ES+): m/z 561 (M+H)⁺.

### Example 175

### 5-((E)-2-(1H-Indazol-4-yl)vinyl)-N-(4-(Piperidin-4-ylsulfonyl)phenyl)pyrimidin-2-amine (Compound LXXXI)

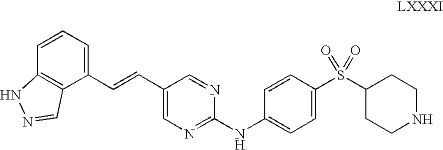

A mixture of intermediate 92 (108 mg, 0.19 mmol) in 3% methanolic HCl (9 mL) was stirred for 3 hours. The solvent was removed in vacuo to afford the HCl salt of The title compound as a yellow solid (102 mg, quantitative).

¹H NMR (500 MHz, DMSO-d₆): δ 1.72 (qd, J=12.9, 3.9 Hz, 2H), 2.02 (d, J=12.6, 2H), 2.85 (ABq, J=12.6 Hz, ν=23.6 Hz, 2H), 3.32 (br d, J=12.1 Hz, 2H), 3.50 (tt, J=11.9, 3.4 Hz, 1H), 7.33-7.38 (m, 2H), 7.42 (d, J=16.7 Hz, 1H), 7.48 (dd, J=7.1, 1.2 Hz, 1H), 7.73 (d, J=16.8 Hz, 1H), 7.76 (d, J=9.0 Hz, 2H), 8.10 (d, J=8.9 Hz, 2H), 8.61 (d, J=0.6 Hz, 1H), 8.62-8.73 (m, 1H), 9.00 (s, 2H), 9.27 (br d, J=10.5 Hz, 1H), 10.49 (s, 1H). MS (ES+): m/z 461 (M+H)⁺.

### Example 176

### 2-[1-(4-Bromo-benzenesulfonyl)-piperidin-4-yl]-ethanol (Intermediate 93)

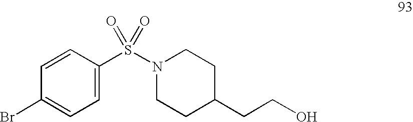

To a solution of 4-bromo-benzenesulfonyl chloride (1.01 g, 3.95 mmol) in DCM (20 mL) was sequentially added 2-(piperidin-4-yl)ethanol (0.59 g, 4.57 mmol), and TEA (1.6 mL, 11.51 mmol). After 30 min, the reaction mixture was washed with sat. NaHCO₃ (25 mL), water (25 mL), and brine (25 mL). The organic layer was dried (Na₂SO₄), filtered, and concentrated in vacuo to afford the title intermediate as a cream solid (1.35 g, 98%).

¹H NMR (500 MHz, DMSO-d₆): δ 1.13 (qd, J=12.1, 3.8 Hz, 2H), 1.29-1.32 (m, 3H), 1.69 (br d, J=11.9 Hz, 2H), 2.21 (td, J=11.9, 2.3 Hz, 2H), 3.38 (ABq, J=6.2 Hz, ν=11.6 Hz, 2H), 3.59 (br d, J=11.8 Hz, 2H), 4.31 (t, J=5.1 Hz, 1H), 7.66 (d, J=8.6 Hz, 2H), 7.85 (d, J=8.3 Hz, 2H). MS (ES+): m/z 348/350 (M+H)⁺.

### Example 177

### 2-[1-(4-{5-[2-(3-Methoxy-phenyl)-vinyl]-pyrimidin-2-ylamino}-benzenesulfonyl)-piperidin-4-yl]-ethanol (Intermediate 94)

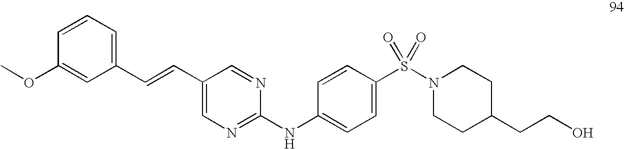

A mixture of intermediate 13 (136 mg, 0.60 mmol), intermediate 93 (225 mg, 0.65 mmol), Pd₂(dba)₃ (27 mg, 0.03 mmol), xantphos (45 mg, 0.08 mmol) and Cs₂CO₃ (391 mg, 1.20 mmol) in dioxane (3 mL) was irradiated in the microwave at 160° C. for 15 minutes. The reaction was filtered, the filtrate concentrated in vacuo, and the resulting crude material purified by gradient flash chromatography (0-100% EtOAC/hexanes) to afford the title intermediate as a beige solid (142 mg, 48%).

¹H NMR (500 MHz, DMSO-d₆): δ 1.09-1.18 (m, 2H), 1.26-1.34 (m, 5H), 1.69 (br d, J=11.1 Hz, 2H), 2.17 (br t, J=11.0 Hz, 2H), 3.36-3.39 (m, under water peak), 3.57 (br d, J=11.5 Hz, 2H), 3.80 (s, 3H), 4.30 (t, J=5.1 Hz, 1H), 6.85-6.87 (m, 1H), 7.08-7.09 (m, 1H), 7.14-7.21 (m, 3H), 7.29-7.33 (m, 2H), 7.65 (d, J=8.9 Hz, 2H), 8.02 (d, J=8.9 Hz, 2H), 8.83 (s, 2H), 10.34 (s, 1H). MS (ES+): m/z 495 (M+H)⁺.

### Example 178

### 3-[2-(2-{4-[4-(2-Hydroxy-ethyl)-piperidine-1-sulfonyl]-phenylamino}-pyrimidin-5-yl)-vinyl]-phenol (Compound LXXXII)

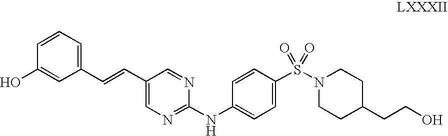

To a solution of intermediate 94 (142 mg, 0.29 mmol) in DCM (10 mL) was added BBr₃ (108 μL, 1.15 mmol) and the reaction was stirred for 10 minutes. MeOH was added to quench the reaction and the mixture was concentrated in vacuo. The crude material was purified by preparative HPLC. The aqueous fractions were extracted with EtOAc (2×50 mL) and the combined organic layers were concentrated in vacuo, triturated with EtOAc/DCM/hexanes, and filtered to obtain The title compound as a beige solid (26 mg, 19%).

¹H NMR (500 MHz, DMSO-d₆): δ 1.09-1.18 (m, 2H), 1.26-1.34 (m, 2H), 1.69 (br d, J=11.5 Hz, 2H), 2.16 (br t, J=10.9 Hz, 2H), 3.38 (Abq, J=6.0 Hz, ν=11.3 Hz, 2H), 3.57 (br d, J=11.5 Hz, 2H), 4.30 (t, J=5.1 Hz, 1H), 6.69 (dd, J=8.0, 2.0 Hz, 1H), 7.01 (s, 1H), 7.08 (d, J=16.6 Hz, 1H), 7.18 (t, J=7.8 Hz, 1H), 7.25 (d, J=16.6 Hz, 1H), 7.65 (d, J=8.9 Hz, 2H), 8.02 (d, J=8.9 Hz, 2H), 8.83 (s, 2H), 9.45 (s, 1H), 10.33 (s, 1H). MS (ES+): m/z 481 (M+H)⁺.

### Example 179

### 4-Bromo-N-(2-pyrrolidin-1-yl-ethyl)-benzenesulfonamide (Intermediate 95)

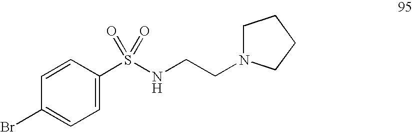

4-bromo-benzenesulfonyl chloride (3.36 g, 13.1 mmol, 1 equivalent) was dissolved in 50 mL DCM and treated with TEA (9.16 mL, 65.7 mmol, 5 equivalent). To this, while stirring the solution, was added 2-pyrrolidin-1-yl-ethylamine (3 g, 26.3 mmol, 2 equivalent). After 3 hours, reaction was poured onto DCM/water mixture and washed once. The aqueous phase was back extracted once with fresh DCM. Organic phases were combined, washed once with brine and dried over sodium sulfate. Filtration followed by rotary evaporation provided The title intermediate. White needles (3.92 g, 90% yield).

### Example 180

### N-(2-Pyrrolidin-1-yl-ethyl)-4-(5-vinyl-pyrimidin-2-ylamino)-benzenesulfonamide (Intermediate 96)

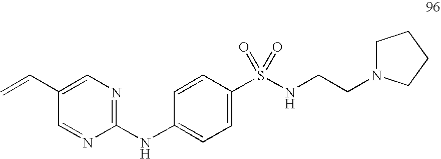

A mixture of intermediate 1 (188 mg, 1.55 mmol), intermediate 95 (505 mg, 1.52 mmol), Pd₂(dba)₃ (72 mg, 0.08 mmol), xantphos (106 mg, 0.18 mmol) and Cs₂CO₃ (980 mg, 3.01 mmol) in dioxane (15 mL) was irradiated in the microwave at 160° C. for 15 minutes. The reaction was concentrated in vacuo, taken up in MeOH, filtered, and the resulting crude material purified by gradient flash chromatography (0-20% MeOH in DCM with 0.5% ammonium hydroxide) to afford the title intermediate as a pale-yellow solid (315 mg, 56%). MS (ES+): m/z 374 (M+H)⁺.

### Example 181

### 4-{5-[2-(1H-Indazol-4-yl)-vinyl]-pyrimidin-2-ylamino}-N-(2-Pyrrolidin-1-yl-ethyl)-benzenesulfonamide (Compound LXXXIII)

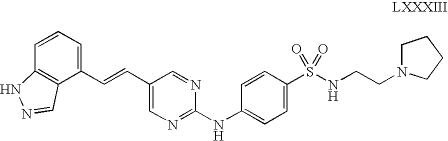

A mixture of 4-bromo-1H-indazole (81 mg, 0.41 mmol), intermediate 96 (101 mg, 0.27 mmol), Pd(OAc)₂ (3.3 mg, 0.02 mmol), PPh₃ (8.7 mg, 0.03 mmol), TEA (186 μL, 1.34 mmol) in DMF (1.5 mL) was irradiated in the microwave at 180° C. for 20 minutes. The reaction was filtered, the filtrate was purified by preparative HPLC, and the resulting fractions were concentrated in vacuo to obtain the TFA salt of The title compound as a yellow solid (48 mg, 30%).

¹H NMR (500 MHz, DMSO-d₆): δ 1.83-1.89 (m, 2H), 1.97-2.04 (m, 2H), 3.04 (br q, J=6.5 Hz, 4H), 3.23 (q, J=6.2 Hz, 2H), 7.35-7.39 (m, 2H), 7.42 (d, J=16.7 Hz, 1H), 7.48 (br d, J=7.3 Hz, 1H), 7.71 (d, J=16.8 Hz, 1H), 7.76-7.79 (m, 3H), 8.04 (d, J=8.9 Hz, 2H), 8.60 (s, 1H), 8.98 (s, 2H), 9.53 (br s, 1H), 10.37 (s, 1H), 13.20 (br s, 1H). MS (ES+): m/z 490 (M+H)⁺.

### Example 182

### N-(4-(Piperidin-4-ylsulfonyl)phenyl)-5-vinylpyrimidin-2-amine (Compound LXXXIV)

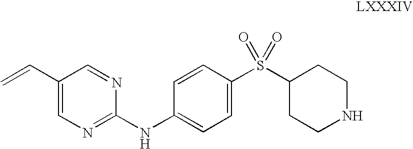

A mixture of intermediate 6 (113 mg, 0.25 mmol) in 30% TFA/DCM (3 mL) was stirred for 5 minutes. The reaction mixture was concentrated in vacuo and purified by preparative HPLC. The resulting fractions were concentrated in vacuo to obtain the TFA salt of The title compound as a white solid (88 mg, 75%).

¹H NMR (500 MHz, DMSO-d₆): δ 1.66 (qd, J=13.0, 3.7 Hz, 2H), 2.02 (d, J=12.8, 2H), 2.87 (td, J=12.8, 2.4 Hz, 2H), 3.36 (br s, under water peak), 3.47 (tt, J=12.0, 3.5 Hz, 1H), 5.31 (d, J=11.2 Hz, 1H), 5.94 (d, J=17.9 Hz, 1H), 6.67 (dd, J=17.8, 11.2 Hz, 1H), 7.75 (d, J=8.8 Hz, 2H), 8.06 (d, J=8.8 Hz, 2H), 8.43 (br s, 2H), 8.74 (s, 2H), 10.42 (s, 1H). MS (ES+): m/z 345 (M+H)⁺.

### Example 183

### tert-Butyl (S)-1-(4-(4-(5-((E)-2-(1H-indol-4-yl)vinyl)pyrimidin-2-ylamino)phenylsulfonyl)piperidin-1-yl)-1-oxopropan-2-ylcarbamate (Intermediate 97)

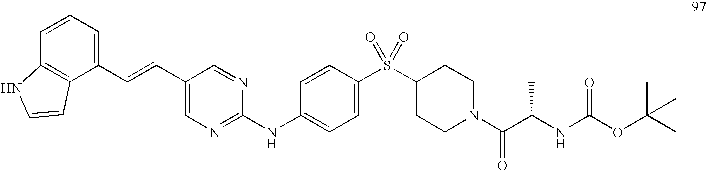

To a solution of 2-tert-butoxycarbonylamino-propionic acid (96 mg, 0.51 mmol) in DMF (4 mL) was added HBTU (255 mg, 0.67 mmol), DIEA (227 μL, 1.30 mmol), and the above-described compound LXXX (200 mg, 0.44 mmol). The reaction was stirred for 5 min and concentrated in vacuo. The crude reaction mixture was purified by gradient flash chromatography (0-100% EtOAc/hexanes) to afford the title intermediate as a beige solid (303 mg, 95%). MS (ES+): m/z 631 (M+H)⁺.

### Example 184

### (S)-1-(4-(4-(5-((E)-2-(1H-Indol-4-yl)vinyl)pyrimidin-2-ylamino)phenylsulfonyl)piperidin-1-yl)-2-aminopropan-1-one (LXXXV)

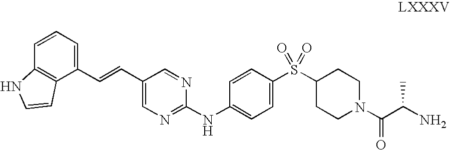

A mixture of intermediate 97 (302 mg, 0.48 mmol) in 30% TFA/DCM (6 mL) was stirred for 20 minutes. The reaction mixture was concentrated in vacuo and purified by preparative HPLC. The resulting fractions were concentrated in vacuo and the residue taken up in MeOH. The TFA salt solution was stirred for 16 h in the presence of Amberlite IRA-400 (Cl⁻) ion exchange resin (5 g). The resin was filtered and the solution concentrated in vacuo to afford the HCl salt of The title compound as a yellow-brown solid (93 mg, 34%).

¹H NMR (500 MHz, DMSO-d₆): δ 1.22-1.38 (m, 5H), 1.45-1.54 (m, 1H), 1.87-1.98 (m, 2H), 2.62-2.71 (m, 1H), 3.04-3.14 (m, 4H), 3.93 (br d, J=14.2 Hz, 1H), 4.26-4.48 (m, 2H), 6.94 (s, 1H), 7.12 (t, J=7.7 Hz, 1H), 7.27 (d, J=16.6 Hz, 1H), 7.32 (d, J=7.4 Hz, 1H), 7.35 (d, J=8.1 Hz, 1H), 7.44 (t, J=2.8 Hz, 1H), 7.70 (d, J=16.7 Hz, 1H), 7.76 (t, J=7.9 Hz, 2H), 8.02-8.18 (m, 6H), 8.96 (s, 2H), 10.42 (s, 1H), 11.26 (s, 1H).

### Example 185

### 2-Bromo-4-fluoro-6-nitrobenzenamine (Intermediate 98)

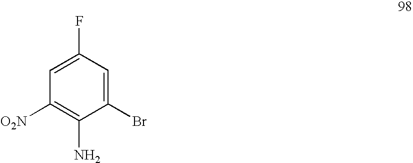

To an argon purged solution of 4-fluoro-2-nitrobenzenamine (2.00 g, 12.8 mmol) in DMF (64 mL) was added dropwise a solution of NBS (2.28 g, 12.8 mmol) in DMF (64 mL) over 20 minutes. The reaction was stirred for 18 h and poured into water (400 mL). The aqueous solution was extracted with DCM (4×100 mL), and the combined organic layers were washed with water (3×40 mL), dried (MgSO₄), filtered, and concentrated in vacuo. The crude residue was purified by gradient flash chromatography (0-60% EtOAc/hexanes) to afford the title intermediate as an orange solid (2.17 g, 72%). R_{f}=0.72, 70% EtOAc/hexanes. ¹H NMR (500 MHz, DMSO-d₆): δ 7.10 (br s, 2H), 7.93 (dd, J=9.2, 2.9 Hz, 1H), 8.02 (dd, J=7.5, 3.0 Hz, 1H).

### Example 186

### 3-Bromo-5-fluorobenzene-1,2-diamine (Intermediate 99)

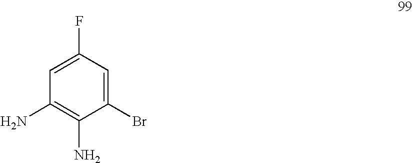

A mixture of a intermediate 98 (545 mg, 2.32 mmol) and tin(II) chloride dihydrate (2.64 g, 11.70 mmol) in 2:1 EtOAc/EtOH (9 mL) was heated to 70° C. for 5 h. The cooled reaction mixture was poured into ice (˜20 mL), and NaHCO₃ was added until the pH reached 7-8. The solid precipitate was filtered and rinsed with EtOAc. The layers were separated and the aqueous phase extracted with EtOAc (3×50 mL). The combined organic layers were washed with brine (25 mL), dried (Na₂SO₄), and concentrated to afford a brown oil that crystallized on standing. The crude solid was further purified by gradient flash chromatography (0-60% EtOAc/hexanes) to afford the title intermediate as a brown oil that crystallized on standing (397 mg, 84%). ¹H NMR (500 MHz, DMSO-d₆): δ 4.45 (s, 2H), 5.20 (s, 2H), 6.36 (dd, J=10.8, 2.9 Hz, 1H), 6.50 (dd, J=8.4, 2.7 Hz, 1H).

### Example 187

### 4-Bromo-6-fluoro-1H-benzo[d][1,2,3]triazole (Intermediate 100)

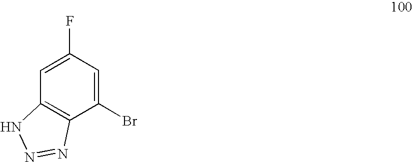

To a solution of intermediate 99 (263 mg, 1.28 mmol) and conc. HCl (1.2 mL) in water (12 mL) was added dropwise a solution of sodium nitrite (0.91 g, 13.19 mmol) in water (3.5 mL) over 3 minutes. The reaction was stirred for 10 minutes and extracted with EtOAc (20 mL). The organic phase was concentrated in vacuo and the residue purified by gradient flash chromatography (0-20% MeOH in DCM) to afford the title intermediate as a white solid (177 mg, 64%). MS (ES+): m/z 216/218 (M+H)⁺.

### Example 188

### tert-Butyl 4-(4-(5-((E)-2-(6-fluoro-1H-benzo[d][1,2,3]triazol-4-yl)vinyl)pyrimidin-2-ylamino)phenylsulfonyl)piperidine-1-carboxylate (Intermediate 101)

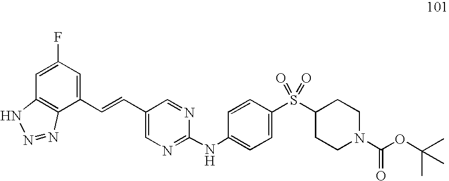

A mixture of intermediate 100 (177 mg, 0.82 mmol), intermediate 6 (246 mg, 0.55 mmol), Pd(OAc)₂ (6 mg, 0.03 mmol), PPh₃ (23 mg, 0.09 mmol), TEA (304 μL, 2.19 mmol) in DMF (4 mL) was irradiated in the microwave at 180° C. for 15 minutes. The reaction had only partially proceeded as determined by LC-MS. A further portion of Pd(OAc)₂ (15 mg, 0.07 mmol) was added and the reaction resubmitted to microwave irradiation at 180° C. for 30 minutes. The reaction was filtered, and the filtrate was purified by preparative HPLC to obtain The title intermediate as a yellow-brown solid (31 mg, 10%). MS (ES+): m/z 580 (M+H)⁺.

### Example 189

### 5-((E)-2-(6-Fluoro-1H-benzo[d][1,2,3]triazol-4-yl)vinyl)-N-(4-(piperidin-4-ylsulfonyl)phenyl)pyrimidin-2-amine (Compound LXXXVI)

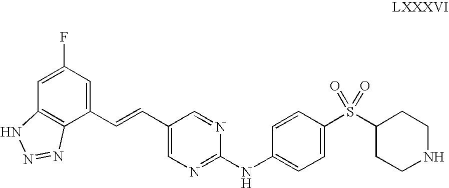

A mixture of intermediate 101 (31 mg, 0.05 mmol) in 30% TFA/DCM (3 mL) was stirred for 5 minutes. The reaction mixture was concentrated in vacuo and purified by preparative HPLC. The resulting fractions were concentrated in vacuo to obtain the TFA salt of The title compound as a yellow solid (11 mg, 33%).

¹H NMR (500 MHz, DMSO-d₆): δ 1.73 (qd, J=13.3, 3.3 Hz, 2H), 2.07 (br d, J=12.6, 2H), 2.86-2.96 (m, 3H), 3.37 (br d, J=12.9 Hz, under water peak), 7.44 (br d, J=10.1 Hz, 1H), 7.50-7.57 (m, 1H), 7.68 (d, J=16.6 Hz, 1H), 7.78 (d, J=8.8 Hz, 2H), 8.11 (d, J=8.9 Hz, 2H), 8.19 (br s, 1H), 8.61 (br s, 1H), 8.93 (s, 2H), 10.38 (s, 1H). MS (ES+): m/z 480 (M+H)⁺.

### Example 190

### tert-Butyl 2-(4-(4-(5-((E)-2-(1H-indol-4-yl)vinyl)pyrimidin-2-ylamino)phenylsulfonyl)piperidin-1-yl)-2-oxoethylcarbamate (Intermediate 102)

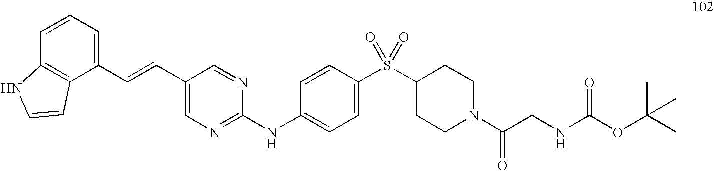

To a solution of tert-butoxycarbonylamino-acetic acid (93 mg, 0.53 mmol) in DMF (3 mL) was added HBTU (257 mg, 0.68 mmol), DIEA (300 μL, 1.72 mmol), and the above-described compound LXXX (200 mg, 0.44 mmol). The reaction was stirred for 5 min and concentrated in vacuo. The crude reaction mixture was purified by gradient flash chromatography (0-100% EtOAc/hexanes) to afford the title intermediate as a yellow solid (251 mg, 92%).

MS (ES+): m/z 617 (M+H)⁺.

### Example 191

### 1-(4-(4-(5-((E)-2-(1H-Indol-4-yl)vinyl)pyrimidin-2-ylamino)phenylsulfonyl)piperidin-1-yl)-2-aminoethanone (Compound LXXXVII)

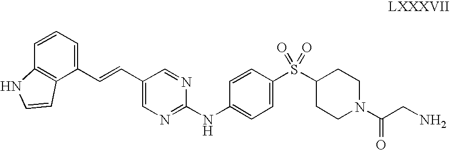

A solution of intermediate 102 (251 mg, 0.41 mmol) in 30% TFA/DCM (6 mL) was stirred for 20 minutes. DMF (1 mL) was added to the reaction and the mixture was concentrated in vacuo to ˜1 mL. The crude solution was purified by preparative HPLC and the resulting fractions were concentrated in vacuo. The residue was taken up in MeOH and the TFA salt solution was stirred for 16 h in the presence of Amberlite IRA-400 (Cl⁻) ion exchange resin (5 g). The resin was filtered and the solution concentrated in vacuo to afford the HCl salt of The title compound as a yellow-brown solid (64 mg, 12%).

¹H NMR (500 MHz, DMSO-d₆): δ 1.28-1.36 (m, 1H), 1.52-1.55 (m, 1H), 1.88-1.99 (m, 2H), 2.63-2.69 (m, 1H), 3.02-3.09 (m, 5H), 3.74-3.81 (m, 3H), 3.92-3.96 (m, 1H), 4.44 (br d, J=12.4 Hz, 1H), 6.94 (s, 1H), 7.12 (t, J=7.7 Hz, 1H), 7.27 (d, J=16.7 Hz, 1H), 7.32 (d, J=7.3 Hz, 1H), 7.35 (d, J=8.1 Hz, 1H), 7.44 (t, J=2.8 Hz, 1H), 7.70 (d, J=16.7 Hz, 1H), 7.75 (d, J=8.9 Hz, 2H), 8.04-8.07 (m, 6H), 8.08 (d, J=8.9 Hz, 2H), 8.96 (s, 2H), 10.42 (s, 1H), 11.26 (s, 1H). MS (ES+): m/z 517 (M+H)⁺.

### Example 192

### 4-Methyl-5-vinylpyrimidin-2-amine (Intermediate 103)

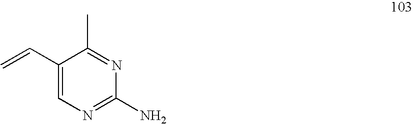

A mixture of intermediate 72 (522 mg, 2.78 mmol), Pd(OAc)₂ (30 mg, 0.13 mmol), PPh₃ (87 mg, 0.33 mmol), TEA (1.54 mL, 11.06 mmol) in DMF (14 mL) was purged with argon for 10 minutes followed by ethylene gas for 10 minutes. The mixture was sealed in a microwave vial and irradiated at 150° C. for 30 minutes. The reaction was filtered, concentrated in vacuo, and the crude material was purified by gradient flash chromatography (0-100% EtOAc/hexanes) to afford the title intermediate as a yellow solid (136 mg, 36%). MS (ES+): m/z 136 (M+H)⁺.

### Example 193

### tert-Butyl 4-(4-(4-methyl-5-vinylpyrimidin-2-ylamino)phenylsulfonyl)piperidine-1-carboxylate (Intermediate 104)

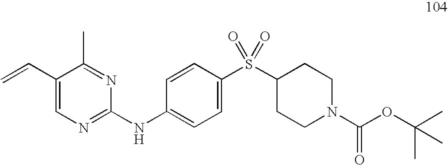

A mixture of intermediate 103 (90 mg, 0.67 mmol), intermediate 5 (274 mg, 0.68 mmol), Pd₂(dba)₃ (33 mg, 0.04 mmol), xantphos (50 mg, 0.09 mmol) and Cs₂CO₃ (435 mg, 1.34 mmol) in dioxane (5 mL) was irradiated in the microwave at 160° C. for 20 minutes. The reaction was filtered, rinsed with DCM, and the filtrate concentrated in vacuo. The resulting crude material was purified by gradient flash chromatography (0-100% EtOAc/hexanes) to afford the title intermediate as a white solid (244 mg, 79%).

¹H NMR (500 MHz, DMSO-d₆): δ 1.24-1.32 (m, 4H), 1.36 (s, 9H), 1.83 (br d, J=11.2 Hz, 2H), 2.47 (s, 3H), 2.70 (br s, 2H), 3.34-3.37 (m, under water peak), 3.98 (br s, 2H), 5.33 (d, J=11.3 Hz, 1H), 5.78 (d, J=17.7 Hz, 1H), 6.82 (dd, J=17.6, 11.2 Hz, 1H), 7.71 (d, J=8.8 Hz, 2H), 8.06 (d, J=8.8 Hz, 2H), 8.67 (s, 1H), 10.27 (s, 1H). MS (ES+): m/z 459 (M+H)⁺.

### Example 194

### tert-Butyl 4-(4-(5-(3-methoxystyryl)-4-methylpyrimidin-2-ylamino)phenylsulfonyl)piperidine-1-carboxylate (Intermediate 105)

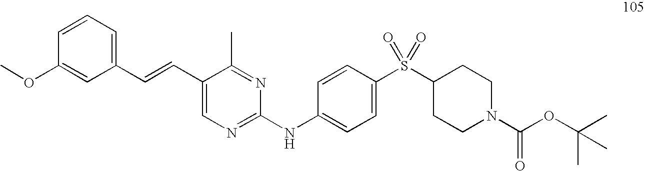

A mixture of 1-bromo-3-methoxybenzene (66.7 μL, 0.53 mmol), intermediate 104 (244 mg, 0.53 mmol), Pd(OAc)₂ (6 mg, 0.03 mmol), PPh₃ (19 mg, 0.07 mmol), TEA (295 μL, 2.12 mmol) in DMF (3 mL) was irradiated in the microwave at 150° C. for 15 minutes. The reaction had only partial proceeded as determined by LC-MS. Further addition of 1-bromo-3-methoxybenzene and irradiation failed to progress the reaction any further. The reaction was filtered, rinsed with DCM, and the filtrate concentrated in vacuo. The resulting crude material was purified by gradient flash chromatography (0-70% EtOAc/hexanes) to afford the title intermediate as a cream solid (224 mg, 75%). MS (ES+): m/z 565 (M+H)⁺.

### Example 195

### 3-((E)-2-(2-(4-(Piperidin-4-ylsulfonyl)phenylamino)-4-methylpyrimidin-5-yl)vinyl)phenol (Compound LXXXVIII)

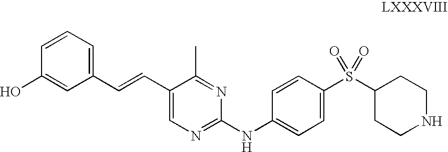

To a solution of intermediate 105 (131 mg, 0.23 mmol) in DCM (2 mL) was added BBr₃ (66 μL, 0.70 mmol), and the reaction was stirred for 5 hours. MeOH was added to quench the reaction, and the mixture was concentrated in vacuo. The crude material was purified by preparative HPLC, and conc. NaHCO₃ was added to the resulting fractions until a pH of 8 was attained. The basic aqueous fractions were extracted with EtOAc (2×75 mL), EtOAc/MeOH (95:5, 1×75 mL), DCM (1×75 mL), EtOAc (1×75 mL). The combined organic layers were washed with brine (1×20 mL), dried (Na₂SO₄), filtered, and concentrated in vacuo. The residue was suspended in MeOH and 3 drops of conc. HCl were added. The mixture was concentrated in vacuo to obtain the HCl salt of The title compound as a yellow solid (17 mg, 15%).

¹H NMR (500 MHz, DMSO-d₆): δ 1.71 (qd, J=12.6, 3.4 Hz, 2H), 2.01 (d, J=12.4, 2H), 2.57 (s, 3H), 2.81-2.86 (m, 2H), 3.31 (br d, J=12.8 Hz, 2H), 3.46-3.51 (m, 1H), 6.71 (dd, J=8.0, 2.0 Hz, 1H), 7.01 (s, 1H), 7.06 (d, J=7.7 Hz, 1H), 7.11 (d, J=16.4 Hz, 1H), 7.18 (t, J=7.8 Hz, 1H), 7.19 (d, J=16.4 Hz, 1H), 7.73 (d, J=8.9 Hz, 2H), 8.10 (d, J=8.8 Hz, 2H), 8.54-8.64 (m, 1H), 8.20 (s, 1H), 9.20 (br d, J=9.8 Hz, 1H), 10.35 (s, 1H). MS (ES+): m/z 451 (M+H)⁺.

### Example 196

### 4-(4-{5-[2-(2-Chloro-5-methoxy-phenyl)-vinyl]-pyrimidin-2-ylamino}-benzenesulfonyl)-piperidine-1-carboxylic acid tert-butyl ester (Intermediate 106)

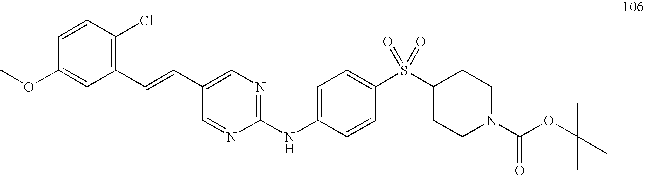

In a dry 15 mL microwave vial were combined 2-bromo-1-chloro-4-methoxy-benzene (0.182 g, 0.82 mmol), intermediate 6 (0.282 g, 0.69 mmol), cesium carbonate (0.896 g, 2.8 mmol), tri-tert-butyl phosphine (1M in toluene) (0.343 mL, 0.343 mmol) and tris(dibenzylideneacetone) dipalladium (0.063 g, 0.068 mmol). Reactants were diluted with dioxane (6 mL), flushed with argon and irradiated for 15 min at 160° C. Reaction was then spun down, decanted and solvents removed. Residue was then diluted with DCM and adsorbed onto silica gel. Crude product was purified on ISCO normal phase column (80 gram). Beige solids (0.35 g, 87% yield). MS (ES+): m/z 585 (M+H)⁺.

### Example 197

### 4-Chloro-3-(2-{2-[4-(piperidine-4-sulfonyl)-phenylamino]-pyrimidin-5-yl}-vinyl)-phenol (Compound LXXXIX)

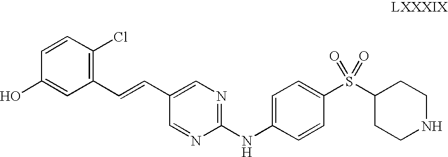

Intermediate 106 (0.35 g, 0.598 mmol) was diluted with 10 mL DCM and chilled to 0° C. A 1.0 M solution of BBr₃ in DCM (4.8 mL, 4.78 mmol) was then added in several portions resulting in dark reaction mixture. Once addition was complete, reaction was allowed to come to ambient temperature and stir for 3 h. Reaction was then quenched by carefully adding an aqueous saturated sodium bicarbonate solution (45 mL). Organic phase was cut from aqueous wash, evaporated and purified by HPLC. Yellow solid (0.017 g, 2% yield).

¹H NMR (DMSO-d₆): δ 1.65 (br s, 2H), 2.04 (br s, 2H), 2.81 (br s, 2H), 6.75 (dd, J=8.7, 2.9 Hz, 1H), 7.13 (d, J=16.0 Hz, 1H), 7.18 (d, J=2.9 Hz, 1H), 7.27 (d, J=8.8 Hz, 1H), 7.42 (d, J=16 Hz, 1H), 7.76 (d, J=8.9 Hz, 1H), 8.08 (d, J=8.8 Hz, 1H), 8.88 (s, 2H), 9.77 (s, 1H), 10.5 (s, 1H). MS (ES+): m/z 471 (M+H)⁺.

### Example 198

### 4-(3-Bromo-phenyl)-piperazine-1-carboxylic acid tert-butyl ester (Intermediate 107)

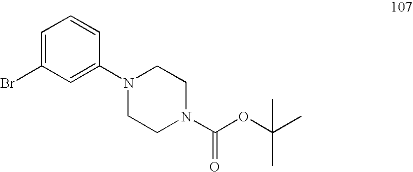

1-(3-Bromo-phenyl)-piperazine (1.0 g, 4.2 mmol) was diluted with DCM (20 mL) and treated with TEA (1.5 mL, 10.4 mmol) and di-tert-butyl dicarbonate (1.15 mL, 4.9 mmol) then refluxed for 18 h. Reaction was then cooled, diluted with DCM (ca. 100 mL) and washed successively with water (1×150 mL) then brine (1×150 mL). Organic phase was dried over Na₂SO₄, filtered and evaporated to provide The title intermediate 107 as pale yellow oil (1.2 g, 85%).

### Example 199

### 4-(3-{5-[2-(1H-Indol-4-yl)-vinyl]-pyrimidin-2-ylamino}-phenyl)-piperazine-1-carboxylic acid tert-butyl ester (Intermediate 108)

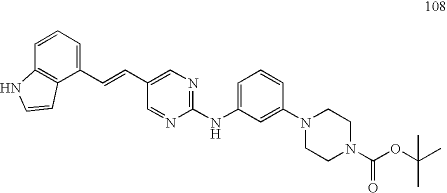

In a dry 15 mL microwave vial were combined intermediate 11 (0.081 g, 0.34 mmol), intermediate 107 (0.140 g, 0.41 mmol), cesium carbonate (0.336 g, 1 mmol), Xantphos (0.04 g, 0.068 mmol) and tris(dibenzylideneacetone) dipalladium (0.031 g, 0.034 mmol) were combined. Reactants were diluted with dioxane (6 mL), flushed with argon and irradiated for 15 min at 160° C. Reaction was then spun down, decanted and solvents removed. Crude product was purified via HPLC. Pure fractions were combined, diluted with EtOAc (ca. 150 mL) and neutralized with saturated aqueous sodium bicarbonate solution. Organic phase was dried over Na₂SO₄, filtered and evaporated to yield yellow solids (0.031 g, 18%).

### Example 200

### {5-[2-(1H-Indol-4-yl)-vinyl]-pyrimidin-2-yl}-(3-piperazin-1-yl-phenyl)-amine (Compound XC)

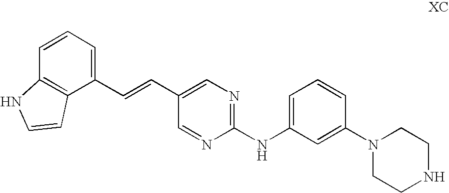

A stirring solution of intermediate 108 (0.031 g, 0.063 mmol) in DCM (5 mL) was treated with TFA (0.2 mL) and allowed to stir at room temperature for 24 h. Solvents were then removed and crude residue purified on HPLC. Pure fractions were combined and evaporated to afford the title product as a yellow powder (0.01 g, 40%).

¹H NMR (DMSO-d₆): δ 3.27 (br s, 4H), 3.32-3.34 (m, 4H), 6.63 (dd, J=8.2, 2.1 Hz, 1H), 6.92 (s, 1H), 7.11 (t, J=7.7 Hz, 1H), 7.17-7.23 (m, 2H), 7.29-7.35 (m, 3H), 7.43 (t, J=2.8 Hz, 1H), 7.48 (s, 1H), 7.61 (d, J=16.7 Hz, 1H), 8.75 (s, 2H), 8.84 (s, 2H), 9.67 (s, 1H), 11.21 (s, 1H). MS (ES+): m/z 397 (M+H)⁺.

### Example 201

### 4-(4-Bromo-phenyl)-piperidine-1-carboxylic acid tert-butyl ester (Intermediate 109)

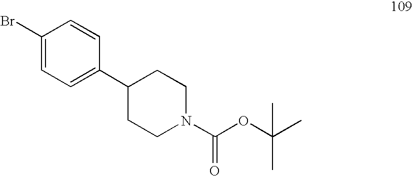

4-(4-Bromo-phenyl)-piperidine (0.323 g, 1.3 mmol) was diluted with DCM (10 mL) and treated with TEA (0.5 mL, 3.4 mmol) and di-tert-butyl dicarbonate (0.352 mL, 1.6 mmol) then refluxed for 1 h. Reaction was then cooled, diluted with DCM (ca. 100 mL) and washed successively with water (1×150 mL) then brine (1×150 mL). Organic phase was dried over Na₂SO₄, filtered and evaporated to provide The title intermediate as pale yellow oil (0.35 g, 76%).

### Example 202

### 4-(4-{5-[2-(1H-Indol-4-yl)-vinyl]-pyrimidin-2-ylamino}-phenyl)-piperidine-1-carboxylic acid tert-butyl ester (Intermediate 110)

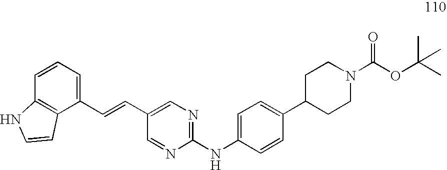

A mixture of intermediate 109 (0.229 g, 0.67 mmol), intermediate 11 (0.132 g, 0.56 mmol), Pd₂(dba)₃ (0.051 g, 0.056 mmol), Xantphos (0.065 g, 0.112 mmol) and cesium carbonate (0.549 g, 1.68 mmol) were suspended in dioxane (10 mL), sealed in a microwave reaction tube and irradiated with microwaves at 160° C. for 15 min. The reaction mixture was cooled to room temperature and centrifuged down. The reaction was decanted and the organic phase concentrated in vacuo. The residue was purified by silica gel chromatography (5%-50% EtOAc in Hexanes). The title intermediate was isolated as yellow solids (0.095 g, 34%).

### Example 203

### {5-[2-(1H-Indol-4-yl)-vinyl]-pyrimidin-2-yl}-(4-piperidin-4-yl-phenyl)-amine (Compound XCI)

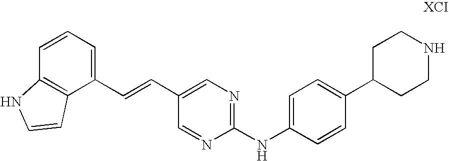

A stirring solution of intermediate 110 (0.09 g, 0.018 mmol) in DCM (10 mL) was treated with TFA (0.5 mL) and allowed to stir at room temperature for 2 h. Solvents were then removed and residue diluted with minimum amount of EtOAc and precipitated out by addition of hexanes. Yellow solids were filtered off and dried (0.041 g, 57%).

¹H NMR (DMSO-d₆): δ 1.75-1.83 (m, 2H), 1.93 (d, J=14.0 Hz, 1H), 2.77-2.81 (m, 1H), 2.96-3.03 (m, 3H), 3.37 (d, J=12.0 Hz, 1H), 6.92 (br s, 1H), 7.11 (t, J=7.7 Hz, 1H), 7.16 (d, J=8.7 Hz, 2H), 7.22 (d, J=15.0 Hz, 1H), 7.29-7.34 (m, 2H), 7.42 (t, J=2.8 Hz, 1H), 7.60 (d, J=15.0 Hz, 1H), 7.73 (d, J=8.6 Hz, 2H), 8.48 (br s, 1H), 8.72 (br s, 1H), 8.83 (s, 2H), 9.75 (s, 1H), 11.2 (s, 1H). MS (ES+): m/z 396 (M+H)⁺.

### Example 204

### 4-(4-{5-[2-(3-Carbamimidoyl-phenyl)-vinyl]-pyrimidin-2-ylamino}-benzenesulfonyl)-piperidine-1-carboxylic acid tert-butyl ester (Intermediate 111)

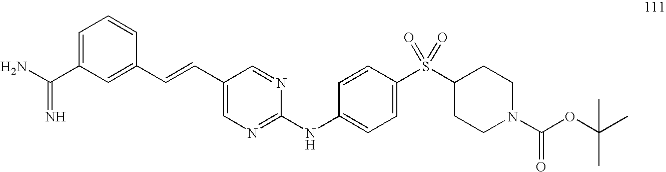

A mixture of intermediate 6 (0.099 g, 0.67 mmol), 3-bromo-benzamidine (0.047 g, 0.198 mmol), Pd(OAc)₂ (0.0022 g, 0.01 mmol), triphenyl phosphine (0.0052 g, 0.02 mmol) and triethylamine (0.027 mL, 0.2 mmol) were suspended in DMF (4 mL), sealed in a microwave reaction tube and irradiated with microwaves at 200° C. for 15 min. The reaction mixture was cooled to room temperature and centrifuged down. The reaction was decanted and the organic phase concentrated in vacuo. The residue was purified by HPLC. The title intermediate was isolated as yellow solids (0.025 g, 22%).

### Example 205

### 3-(2-{2-[4-(Piperidine-4-sulfonyl)-phenylamino]-pyrimidin-5-yl}-vinyl)-benzamidine (Compound XCII)

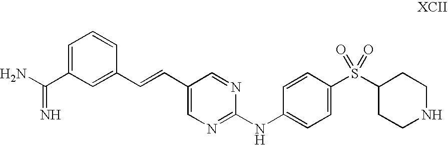

A stirring solution of intermediate 111 (0.025 g, 0.044 mmol) in DCM (10 mL) was treated with TFA (0.4 mL) and allowed to stir at room temperature for 18 h. Solvents were then removed and the residue was purified by HPLC. Pure fractions were combined and evaporated to afford the title compound. White solids (0.006 g, 29%).

¹H NMR (DMSO-d₆): δ 1.64-1.71 (m, 2H), 2.02 (d, J=11.7 Hz, 2H), 2.84-2.90 (m, 2H), 6.35 (d, J=16.6 Hz, 1H), 7.44 (d, J=16.6 Hz, 1H), 7.63-7.70 (m, 2H), 7.77 (d, J=9.0 Hz, 2H), 7.89 (d, J=7.8 Hz, 1H), 8.01 (s, 1H), 8.08 (d, J=8.8 Hz, 2H), 8.26 (br s, 1H), 8.70 (br s, 1H), 8.88 (s, 2H), 9.19 (s, 2H), 9.38 (s, 2H), 10.51 (s, 1H)

MS (ES+): m/z 463 (M+H)⁺.

### Example 206

### 4-Bromo-N-(3-hydroxy-propyl)-benzenesulfonamide (Intermediate 112)

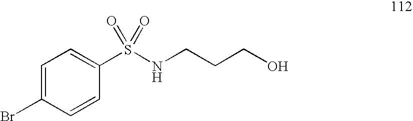

A stirring solution of 4-bromo-benzenesulfonyl chloride (2.53 g, 11.2 mmol) in DCM (30 mL) was treated with triethylamine (3.8 mL, 28 mmol) and chilled to 0° C. This was then treated with 3-amino-propan-1-ol (1.72 mL, 22.4 mmol). Reaction was allowed to return to room temperature and stirred for 30 min. This was then poured onto water and washed once. Organic phase was then washed with brine, dried over sodium sulfate, filtered and evaporated to white powder (2.5 g, 76%).

### Example 207

### N-(3-Hydroxy-propyl)-4-{5-[2-(3-methoxy-phenyl)-vin-yl]-pyrimidin-2-ylamino}-benzenesulfonamide (Intermediate 113)

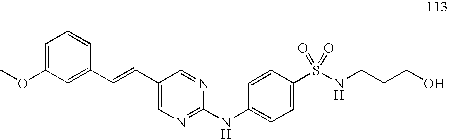

A mixture of intermediate 13 (0.1 g, 0.44 mmol), intermediate 14 (0.162 g, 0.55 mmol), Pd₂(dba)₃ (0.04 g, 0.044 mmol), Xantphos (0.051 g, 0.088 mmol) and cesium carbonate (0.431 g, 1.32 mmol) were suspended in dioxane (10 mL), sealed in a microwave reaction tube and irradiated with microwaves at 160° C. for 15 min. The reaction mixture was cooled to room temperature and centrifuged down. The reaction was then decanted and the organic phase concentrated in vacuo. The residue was purified by silica gel chromatography. The title intermediate was isolated as yellow solids (0.175 g, 90%).

### Example 208

### 4-{5-[2-(3-Hydroxy-phenyl)-vinyl]-pyrimidin-2-ylamino}-N-(3-hydroxy-propyl)-benzenesulfonamide (Compound XCIII)

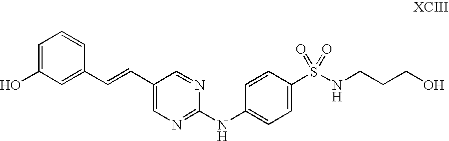

A stirring solution of intermediate 113 (0.175 g, 0.398 mmol) in DCM (10 mL) was treated with BBr₃ (3 mL, 3.18 mmol). After 3 h, reaction was quenched by careful addition of MeOH (10 mL) and evaporated to dryness. The resulting residue was purified by HPLC. Pure fractions were combined, diluted with EtOAc and neutralized with saturated sodium bicarbonate. Organic phase was isolated, dried over sodium sulfate, filtered and evaporated to afford the title compound. White solids (0.003 g, 2%).

¹H NMR (DMSO-d₆): δ 1.48-1.53 (m, 2H), 2.73-2.79 (m, 2H), 3.36 (t, J=6.2 Hz, 1H), 6.69 (dd, J=8.1, 2.3 Hz, 1H), 6.95 (s, 1H), 7.00 (d, J=7.7 Hz, 1H), 7.07 (d, J=16.6 Hz, 1H), 7.18 (t, J=5.6 Hz, 1H), 7.24 (d, J=16.6 Hz, 1H), 7.32 (t, J=5.9 Hz, 1H), 7.69 (d, J=8.8 Hz, 2H), 7.97 (d, J=8.8 Hz, 2H), 8.82 (s, 2H), 9.45 (br s, 1H), 10.26 (s, 1H). MS (ES+): m/z 427 (M+H)⁺.

### Example 209

### 4-(4-{5-[2-(1H-Indol-4-yl)-vinyl]-pyrimidin-2-ylamino}-benzenesulfonylamino)-piperidine-1-carboxylic acid tert-butyl ester (Intermediate 114)

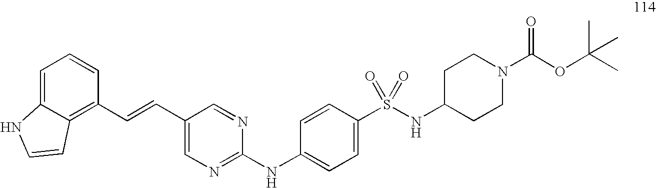

A mixture of intermediate 11 (0.1 g, 0.46 mmol), intermediate 12 (0.242 g, 0.58 mmol), Pd₂(dba)₃ (0.042 g, 0.05 mmol), Xantphos (0.054 g, 0.09 mmol) and cesium carbonate (0.452 g, 1.39 mmol) were suspended in dioxane (10 mL), sealed in a microwave reaction tube and irradiated with microwaves at 160° C. for 15 min. The reaction mixture was cooled to room temperature and centrifuged down. The reaction was then poured onto ice and the resulting yellow solids were filtered and dried (0.2 g, 75%).

### Example 210

### 4-{5-[2-(1H-Indol-4-yl)-vinyl]-pyrimidin-2-ylamino}-N-piperidin-4-yl-benzenesulfonamide (Compound XCIV)

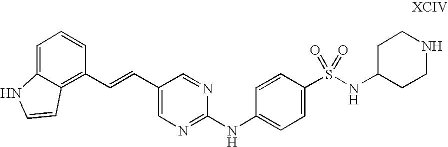

A stirring solution of intermediate 114 (0.2 g, 0.348 mmol) in DCM (10 mL) was treated with TFA (0.4 mL) and allowed to stir at room temperature for 18 h. Solvents were then removed and the residue was purified by HPLC. Pure fractions were combined and evaporated to afford the title compound. White solids, (0.02 g, 12%).

¹H NMR (DMSO-d₆): δ 1.51-1.56 (m, 2H), 1.72-1.76 (m, 2H), 2.87-2.92 (m, 2H), 3.15-3.18 (m, 2H), 6.94 (s, 1H), 7.12 (t, J=7.7 Hz, 1H), 7.26 (d, J=16.7 Hz, 1H), 7.31-7.36 (m, 2H), 7.44 (t, J=2.8 Hz, 1H), 7.67 (d, J=16.7 Hz, 1H), 7.75 (d, J=8.9 Hz, 1H), 7.78 (d, J=6.9 Hz, 1H), 8.00 (d, J=8.9 Hz, 1H), 8.94 (s, 2H), 10.29 (s, 1H), 11.23 (s, 1H). MS (ES+): m/z 475 (M+H)⁺.

### Example 211

### 4-(4-{5-[2-(1H-Indol-4-yl)-vinyl]-pyrimidin-2-ylamino}-benzenesulfonyl)-[1,4]diazepane-1-carboxylic acid tert-butyl ester (Intermediate 115)

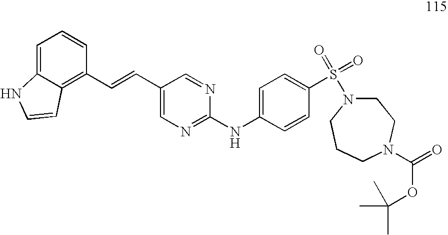

A mixture of intermediate 11 (0.121 g, 0.513 mmol), intermediate 30 (0.27 g, 0.641 mmol), Pd₂(dba)₃ (0.047 g, 0.05 mmol), Xantphos (0.06 g, 0.1 mmol) and cesium carbonate (0.501 g, 1.54 mmol) were suspended in dioxane (10 mL), sealed in a microwave reaction tube and irradiated with microwaves at 160° C. for 15 min. The reaction mixture was cooled to room temperature and centrifuged down. The reaction was then passed through small silica gel plug and evaporated to dryness. The residue was purified by column chromatography providing The title intermediate as beige solids (0.27 g, 92%).

### Example 212

### [4-([1,4]Diazepane-1-sulfonyl)-phenyl]-{5-[2-(1H-indol-4-yl)-vinyl]-pyrimidin-2-yl}-amine (Compound XCV)

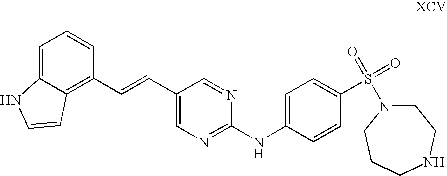

A stirring solution of intermediate 115 (0.27 g, 0.47 mmol) in DCM (10 mL) was treated with TFA (0.8 mL) and allowed to stir at room temperature for 3 h. Solvents were then removed and the residue was purified by HPLC. Pure fractions were combined and evaporated to afford the title compound. White solids, (0.01 g, 4%).

¹H NMR (DMSO-d₆): δ 1.92-1.97 (m, 2H), 3.17 (br s, 2H), 3.20 (br s, 2H), 3.29 (t, J=5.9 Hz, 1H), 3.44-3.46 (m, 2H), 6.93 (s, 1H), 7.12 (t, J=7.7 Hz, 1H), 7.26 (d, J=16.7 Hz, 1H), 7.31-7.36 (m, 2H), 7.44 (t, J=2.8 Hz, 1H), 7.68 (d, J=16.7 Hz, 1H), 7.74 (d, J=8.8 Hz, 1H), 8.05 (d, J=8.9 Hz, 1H), 8.73 (br s, 1H), 8.95 (s, 2H), 10.35 (s, 1H), 11.23 (s, 1H). MS (ES+): m/z 475 (M+H)⁺.

### Example 213

### 1-Bromo-4-(3-chloro-propylsulfanyl)-benzene (Intermediate 116)

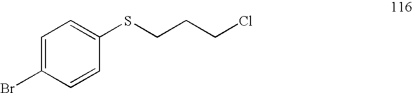

A stirring solution of 4-bromo-benzenethiol (2.66 g, 14 mmol) in dioxane (50 mL) was treated with 1,3-dichloro-propane (1.4 mL, 14 mmol) and cesium carbonate (10 g, 30 mmol). This was then heated to 80° C. and stirred for 16 h. Reaction was then cooled to room temperature and poured onto ice. The resulting oil was collected and purified by column chromatography (0% to 15% EtOAc in hexanes). The title intermediate was isolated as clear oil (1.23 g, 33%).

### Example 214

### 1-Bromo-4-(3-chloro-propane-1-sulfonyl)-benzene (Intermediate 117)

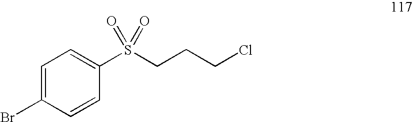

Intermediate 116 (1.2 g, 7.8 mmol) and 3-chloroperbenzoic acid (5.43 g, 31.4 mmol) were combined, diluted with DCM (30 mL) and stirred at room temperature. After 20 h reaction was poured onto water and extracted with EtOAc. Organic phase was washed with saturated sodium bicarbonate, dried over sodium sulfate, filtered and evaporated to dryness (1.68 g, 72%).

### Example 215

### 4-[3-(4-Bromo-benzenesulfonyl)-propyl]-piperazine-1-carboxylic acid tert-butyl ester (Intermediate 118)

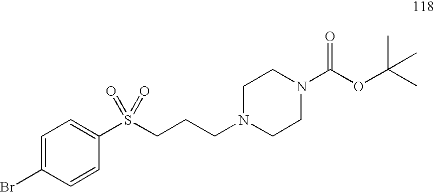

Piperazine-1-carboxylic acid tert-butyl ester (2 g, 11.1 mmol), intermediate 117 (1.1 g, 3.7 mmol) and potassium carbonate (7.2 g, 22.2 mmol) were combined and diluted with DMF (15 mL). Reaction was then sealed in a microwave reaction tube and irradiated with microwaves at 140° C. for 10 min. Reaction was then cooled, poured onto water and extracted with EtOAc. Organic phase was washed with water, brine, dried over sodium sulfate, filtered and evaporated to dryness (0.6 g, 36%).

### Example 216

### 4-[3-(4-{5-[2-(1H-Indol-4-yl)-vinyl]-pyrimidin-2-ylamino}-benzenesulfonyl)-propyl]-piperazine-1-carboxylic acid tert-butyl ester (Intermediate 119)

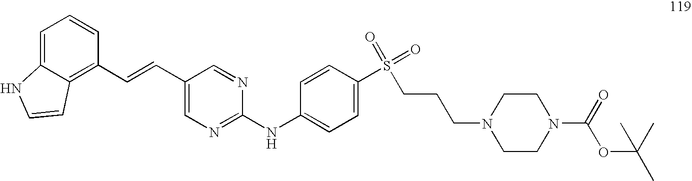

A mixture of intermediate 11 (0.1 g, 0.424 mmol), intermediate 118 (0.237 g, 0.53 mmol), Pd₂(dba)₃ (0.039 g, 0.042 mmol), Xantphos (0.049 g, 0.085 mmol) and cesium carbonate (0.414 g, 1.27 mmol) were suspended in dioxane (10 mL), sealed in a microwave reaction tube and irradiated with microwaves at 160° C. for 15 min. The reaction mixture was cooled to room temperature and centrifuged down. This was then decanted and evaporated to dryness. The residue was purified by column chromatography providing the title intermediate as beige solids (0.056 g, 22%).

### Example 217

### {5-[2-(1H-Indol-4-yl)-vinyl]-pyrimidin-2-yl}-[4-(3-piperazin-1-yl-propane-1-sulfonyl)-phenyl]-amine (Compound XCVI)

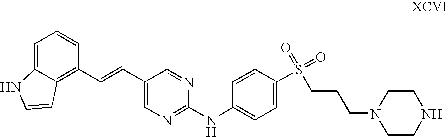

A stirring solution of intermediate 119 (0.056 g, 0.47 mmol) in DCM (10 mL) was treated with TFA (0.5 mL) and allowed to stir at room temperature for 3 h. Solvents were then removed and the residue was purified by HPLC. Pure fractions were combined and evaporated to afford the title compound. White solids (0.004 g, 9%).

¹H NMR (DMSO-d₆): δ 2.02 (br s, 2H), 3.19 (br s, 4H), 3.37-3.40 (m, 4H), 6.94 (s, 1H), 7.12 (t, J=7.6 Hz, 1H), 7.27 (d, J=16.6 Hz, 1H), 7.31-7.36 (m, 2H), 7.44 (t, J=2.8 Hz, 1H), 7.69 (d, J=16.7 Hz, 1H), 7.82 (d, J=8.8 Hz, 1H), 8.08 (d, J=8.9 Hz, 1H), 8.95 (s, 2H), 10.35 (s, 1H), 11.23 (s, 1H). MS (ES+): m/z 503 (M+H)⁺.

### Example 218

### 4-Bromo-2,6-dimethyl-benzenesulfonyl chloride (Intermediate 120)

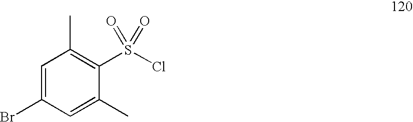

1-Bromo-3,5-dimethyl-benzene (3 g, 16.2 mmol) was chilled to 0° C. and treated with chlorosulfonic acid with vigorous stirring. Reaction appeared to solidify and was removed from ice bath and heated to 70° C. After several min of heating solids melted and resulting red syrup was heated an additional 10 min. Reaction was then cooled to room temperature and diluted with ice water. The resulting beige solids were collected by filtration, washed with water and dried overnight (2.6 g, 57%).

### Example 219

### 4-Bromo-2,6,N-trimethyl-benzenesulfonamide (Intermediate 121)

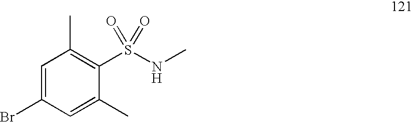

A vigorously stirring solution of methylamine in water (40 wt. %) was treated with intermediate 120 (2.6 g, 9.19 mmol). After 3 h, white precipitate was filtered off and dried (2.37 g, 93%).

### Example 220

### 4-Bromo-2,6,N-trimethyl-N-(2-pyrrolidin-1-yl-ethyl)-benzenesulfonamide (Intermediate 122)

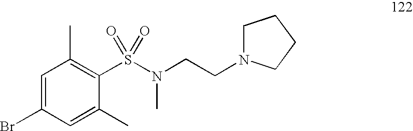

A mixture of intermediate 121 (1.8 g, 6.47 mmol), 1-(2-chloro-ethyl)- and pyrrolidine hydrochloride (3.3 g, 19.42 mmol) and cesium carbonate (10.55 g, 32.37 mmol) was diluted with acetone (50 mL) and heated to reflux. After 15 h, reaction was cooled room temperature, filtered and evaporated to pale brown syrup. This was diluted with EtOAc (200 mL) and washed successively with water and brine. Organic phase was then dried over sodium sulfate, filtered and evaporated to light brown oil (2.4 g, 99%).

### Example 221

### 4-{5-[2-(3-Methoxy-phenyl)-vinyl]-pyrimidin-2-ylamino}-2,6,N-trimethyl-N-(2-pyrrolidin-1-yl-ethyl)-benzenesulfonamide (Intermediate 123)

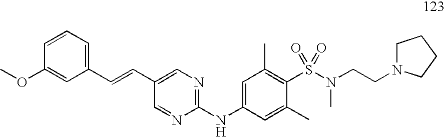

A mixture of immediate 13 (0.15 g, 0.66 mmol), intermediate 122 (0.295 g, 0.79 mmol), Pd₂(dba)₃ (0.066 mmol), Xantphos (0.076 g, 0.13 mmol) and cesium carbonate (0.641 g, 1.97 mmol) were suspended in dioxane (10 mL), sealed in a microwave reaction tube and irradiated with microwaves at 160° C. for 15 min. The reaction mixture was cooled to room temperature and centrifuged down. This was then decanted and evaporated to dryness. The residue was purified by column chromatography providing the title intermediate as beige solids (0.2 g, 58%).

### Example 222

### 4-{5-[2-(3-Hydroxy-phenyl)-vinyl]-pyrimidin-2-ylamino}-2,6,N-trimethyl-N-(2-pyrrolidin-1-yl-ethyl)-benzenesulfonamide (Compound XCVII)

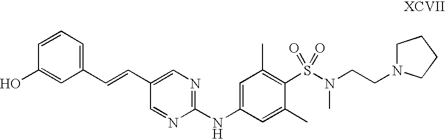

A stirring solution of intermediate 123 (0.2 g, 0.41 mmol) in DCM (10 mL) was treated with BBr₃ (4 mL, 4.1 mmol). After 3 h, reaction was quenched by careful addition of MeOH (10 mL) and evaporated to dryness. The resulting residue was purified by HPLC. Pure fractions were combined, diluted with EtOAc and neutralized with saturated sodium bicarbonate. Organic phase was isolated, dried over sodium sulfate, filtered and evaporated to afford the title compound. Yellow solids (0.017 g, 9%).

¹H NMR (DMSO-d₆): δ 1.83-1.84 (m, 2H), 1.98 (br s, 2H), 2.55 (s, 6H), 2.72 (s, 3H), 2.98 (br s, 2H), 3.35-3.37 (m, 2H), 3.43-3.47 (m, 2H), 6.70 (dd, J=8.0, 1.9 Hz, 1H), 6.95 (s, 1H), 7.00 (d, J=7.9 Hz, 1H), 7.06 (d, J=16.6 Hz, 1H), 7.18 (t, J=7.8 Hz, 1H), 7.25 (d, J=16.6 Hz, 1H), 7.71 (s, 2H), 8.83 (s, 2H), 10.15 (s, 1H), 10.47 (br s, 1H). MS (ES+): m/z 508 (M+H)⁺.

### Example 223

### 4-(4-Bromo-3-methyl-benzenesulfonyl)-piperazine-1-carboxylic acid tert-butyl ester (Intermediate 124)

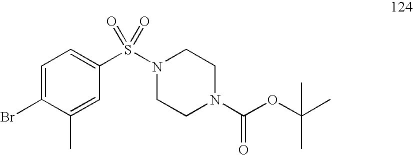

piperazine-1-carboxylic acid tert-butyl ester (1 g, 5.3 mmol) was dissolved in water (45 mL) and treated with 4-bromo-3-methyl-benzenesulfonyl chloride (0.356 g, 1.32 mmol). This mixture was stirred vigorously for 18 h. Solids were then filtered off and dried (0.525 g, 95%).

### Example 224

### 4-(4-{5-[2-(3-Methoxy-phenyl)-vinyl]-pyrimidin-2-ylamino}-3-methyl-benzenesulfonyl)-piperazine-1-carboxylic acid tert-butyl ester (Intermediate 125)

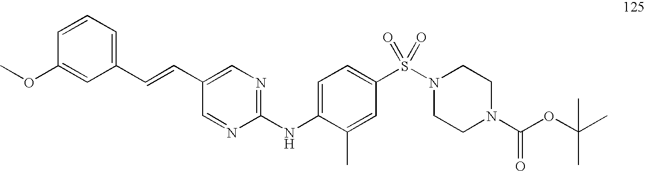

A mixture of intermediate 13 (0.105 g, 0.46 mmol), intermediate 124 (0.233 g, 0.56 mmol), Pd₂(dba)₃ (0.042 g, 0.046 mmol), Xantphos (0.054 g, 0.093 mmol) and cesium carbonate (0.452 g, 1.39 mmol) were suspended in dioxane (10 mL), sealed in a microwave reaction tube and irradiated with microwaves at 160° C. for 15 min. The reaction mixture was cooled to room temperature and centrifuged down. This was then decanted and evaporated to dryness. The residue was purified by column chromatography providing the title intermediate as beige solids (0.11 g, 43%).

### Example 225

### 3-(2-{2-[2-Methyl-4-(piperazine-1-sulfonyl)-phenylamino]-pyrimidin-5-yl}-vinyl)-phenol (Compound XCVIII)

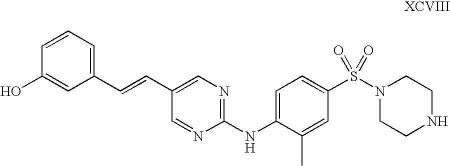

A stirring solution of intermediate 125 (0.11 g, 0.2 mmol) in DCM (10 mL) was treated with BBr₃ (1.6 mL, 1.6 mmol). After 1 h, reaction was quenched by careful addition of MeOH (10 mL) and evaporated to dryness. The resulting residue was purified by HPLC. Pure fractions were combined, diluted with EtOAc and neutralized with saturated sodium bicarbonate. Organic phase was isolated, dried over sodium sulfate, filtered and evaporated to afford the title compound. Yellow solids (0.012 g, 13%).

¹H NMR (DMSO-d₆): δ 2.40 (s, 3H), 3.13-3.14 (m, 4H), 3.19 (br s, 4H), 6.70 (dd, J=8.8, 2.9 Hz, 1H), 6.95 (t, J=1.9 Hz, 1H), 6.99 (d, J=7.9 Hz, 1H), 7.06 (d, J=16.6 Hz, 1H), 7.10 (s, 1H), 7.16-7.20 (m, 2H), 7.25 (d, J=16.6 Hz, 1H), 7.57-7.62 (m, 2H), 8.10 (d, J=8.6 Hz, 1H), 8.77 (s, 2H), 9.07 (br s, 2H), 9.25 (s, 1H). MS (ES+): m/z 452 (M+H)⁺.

### Example 226

### 4-(4-Bromo-2-trifluoromethyl-benzenesulfonyl)-piperazine-1-carboxylic acid tert-butyl ester (Intermediate 126)

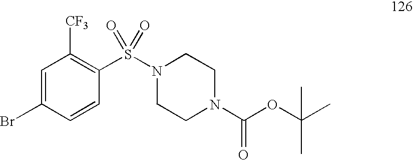

Piperazine-1-carboxylic acid tert-butyl ester (1.9 g, 10.3 mmol) was dissolved in water (45 mL) and treated with 4-bromo-2-trifluoromethyl-benzenesulfonyl chloride (0.66 g, 2.06 mmol). This mixture was stirred vigorously for 18 h. Solids were then filtered off and dried (0.9 g, 93%).

### Example 227

### 4-(4-{5-[2-(3-Methoxy-phenyl)-vinyl]-pyrimidin-2-ylamino}-2-trifluoromethyl-benzenesulfonyl)-piperazine-1-carboxylic acid tert-butyl ester (Intermediate 127)

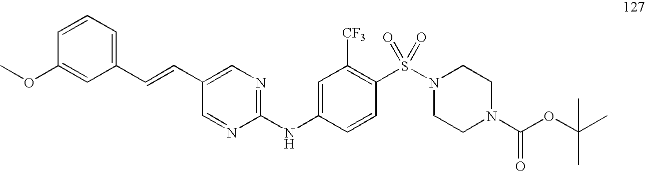

A mixture of intermediate 13 (0.148 g, 0.65 mmol), intermediate 126 (0.37 g, 0.78 mmol), Pd₂(dba)₃ (0.060 g, 0.065 mmol), Xantphos (0.075 g, 0.13 mmol) and cesium carbonate (0.638 g, 1.96 mmol) were suspended in dioxane (10 mL), sealed in a microwave reaction tube and irradiated with microwaves at 160° C. for 15 min. The reaction mixture was cooled to room temperature and centrifuged down. This was then decanted and evaporated to dryness. The resulting orange was used without further purification (0.25 g, 57%).

### Example 228

### 3-(2-{2-[4-(piperazine-4-sulfonyl)-3-trifluoromethyl-phenylamino]-pyrimidin-5-yl}-vinyl)-phenol (Compound XCIX)

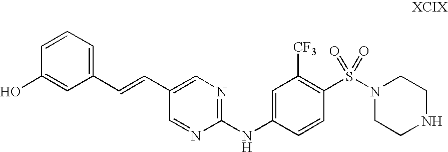

A stirring solution of intermediate 127 (0.25 g, 0.4 mmol) in DCM (10 mL) was treated with BBr₃ (3.2 mL, 3.2 mmol). After 3 h, reaction was quenched by careful addition of aqueous saturated sodium bicarbonate solution (20 mL). Organic phase was then cut from aqueous and evaporated. The resulting residue was purified by HPLC. Pure fractions were combined and diluted with EtOAc and neutralized with saturated sodium bicarbonate. Organic phase was isolated, dried over sodium sulfate, filtered and evaporated to afford the title compound. Yellow solids (0.07 g, 34%).

¹H NMR (DMSO-d₆): δ 3.13 (br s, 4H), 3.38-3.39 (m, 4H), 6.73 (dd, J=8.8, 2.9 Hz, 1H), 6.98-7.01 (m, 2H), 7.08 (d, J=16.6 Hz, 1H), 7.18 (t, J=7.9 Hz, 1H), 7.29 (d, J=16.6 Hz, 1H), 8.04 (d, J=9.0 Hz, 1H), 8.34 (dd, J=9.0, 2.1 Hz, 1H), 8.52 (d, J=2.2 Hz, 1H), 8.89 (s, 2H), 9.67 (br s, 2H), 10.72 (s, 1H). MS (ES+): m/z 506 (M+H)⁺.

### Example 229

### 4-(4-Bromo-2-fluoro-benzenesulfonyl)-piperazine-1-carboxylic acid tert-butyl ester (Intermediate 128)

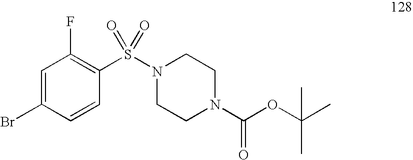

Piperazine-1-carboxylic acid tert-butyl ester (2.15 g, 11.56 mmol) was dissolved in water (40 mL) and treated with 4-bromo-2-fluoro-benzenesulfonyl chloride (0.633 g, 2.3 mmol). This mixture was stirred vigorously for 18 h. Solids were then filtered off and dried (0.9 g, 90%).

### Example 230

### 4-(2-Fluoro-4-{5-[2-(3-methoxy-phenyl)-vinyl]-pyrimidin-2-ylamino}-benzenesulfonyl)-piperazine-1-carboxylic acid tert-butyl ester (Intermediate 129)

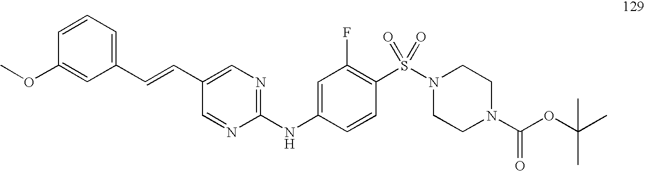

A mixture of intermediate 13 (0.175 g, 0.77 mmol), intermediate 128 (0.39 g, 0.93 mmol), Pd₂(dba)₃ (0.071 g, 0.08 mmol), Xantphos (0.089 g, 0.15 mmol) and cesium carbonate (0.754 g, 2.31 mmol) were suspended in dioxane (10 mL), sealed in a microwave reaction tube and irradiated with microwaves at 160° C. for 15 min. The reaction mixture was cooled to room temperature and centrifuged down. This was then decanted and evaporated to dryness. The resulting residue was used without further purification (0.25 g, 57%).

### Example 231

### 3-(2-{2-[3-Fluoro-4-(piperazine-1-sulfonyl)-phenylamino]-pyrimidin-5-yl}-vinyl)-phenol (Compound C)

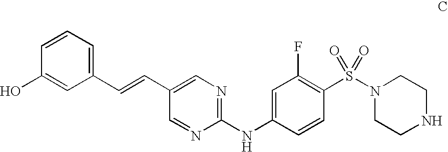

A stirring solution of intermediate 129 (0.25 g, 0.44 mmol) in DCM (10 mL) was treated with BBr₃ (2.6 mL, 2.6 mmol). After 2 h, reaction was quenched by careful addition of MeOH (10 mL) and evaporated to dryness. The resulting residue was purified by HPLC. Pure fractions were combined and diluted with EtOAc and neutralized with saturated sodium bicarbonate. Organic phase was isolated, dried over sodium sulfate, filtered and evaporated to afford the title compound. Yellow solids (0.125 g, 63%).

¹H NMR (DMSO-d₆): δ 3.13 (br s, 4H), 3.29-3.30 (m, 4H), 6.74 (m, 1H), 6.99-7.01 (m, 2H), 7.08 (d, J=16.6 Hz, 1H), 7.17 (t, J=7.9 Hz, 1H), 7.28 (d, J=16.6 Hz, 1H), 7.66-7.74 (m, 2H), 8.34 (d, J=13.9 Hz, 1H), 8.88 (s, 2H), 9.85 (br s, 2H), 10.66 (s, 1H). MS (ES+): m/z 456 (M+H)⁺.

### Example 232

### 4-(4-Bromo-2,5-difluoro-benzenesulfonyl)-piperazine-1-carboxylic acid tert-butyl ester (Intermediate 130)

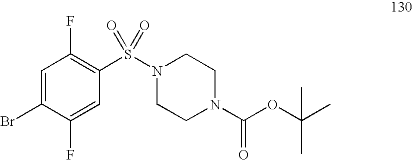

piperazine-1-carboxylic acid tert-butyl ester (1.95 g, 10.5 mmol) was dissolved in water (40 mL) and treated with 4-bromo-2,5-difluoro-benzenesulfonyl chloride (0.612 g, 2.1 mmol). This mixture was stirred vigorously for 18 h. Solids were then filtered off and dried (0.8 g, 87%).

### Example 233

### 4-(2,5-Difluoro-4-{5-[2-(3-methoxy-phenyl)-vinyl]-pyrimidin-2-ylamino}-benzenesulfonyl)-piperazine-1-carboxylic acid tert-butyl ester (Intermediate 131)

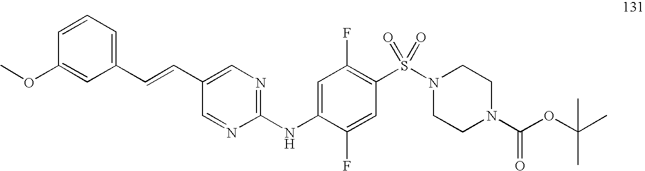

A mixture of intermediate 13 (0.145 g, 0.64 mmol), intermediate 130 (0.34 g, 0.77 mmol), Pd₂(dba)₃ (0.059 g, 0.064 mmol), Xantphos (0.075 g, 0.129 mmol) and cesium carbonate (0.629 g, 1.93 mmol) were suspended in dioxane (10 mL), sealed in a microwave reaction tube and irradiated with microwaves at 160° C. for 15 min. The reaction mixture was cooled to room temperature and centrifuged down. This was then decanted and evaporated to dryness. The resulting residue was used without further purification (0.25 g, 66%).

### Example 234

### 3-(2-{2-[2,5-Difluoro-4-(piperazine-1-sulfonyl)-phenylamino]-pyrimidin-5-yl}-vinyl)-phenol (Compound CI)

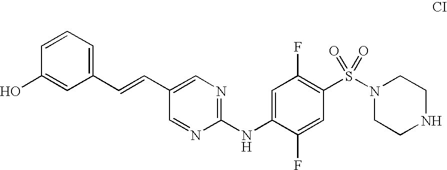

A stirring solution of intermediate 131 (0.25 g, 0.43 mmol) in DCM (10 mL) was treated with BBr₃ (2.6 mL, 2.6 mmol). After 2 h, reaction was quenched by careful addition of MeOH (10 mL) and evaporated to dryness. The resulting residue was purified by HPLC. Pure fractions were combined and diluted with EtOAc and neutralized with saturated sodium bicarbonate. Organic phase was isolated, dried over sodium sulfate, filtered and evaporated to afford the title compound. Yellow solids (0.075 g, 37%).

¹H NMR (DMSO-d₆): δ 3.14 (br s, 4H), 3.35-3.36 (m, 4H), 6.73-6.76 (m, 1H), 6.99-7.01 (m, 2H), 7.09 (d, J=16.8 Hz, 1H), 7.17 (t, J=8.1 Hz, 1H), 7.29 (d, J=16.8 Hz, 1H), 7.59-7.63 (m, 1H), 8.46-8.50 (m, 1H), 8.89 (s, 2H), 9.89 (br s, 2H), 10.08 (s, 1H). MS (ES+): m/z 474 (M+H)⁺.

### Example 235

### 4-(4-Bromo-2-trifluoromethoxy-benzenesulfonyl)-piperazine-1-carboxylic acid tert-butyl ester (Intermediate 132)

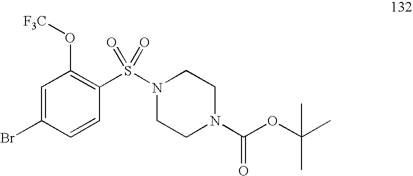

Piperazine-1-carboxylic acid tert-butyl ester (2.11 g, 11.34 mmol) was dissolved in water (40 mL) and treated with 4-bromo-2-trifluoromethoxy-benzenesulfonyl chloride (0.77 g, 2.27 mmol). This mixture was stirred vigorously for 18 h. Solids were then filtered off and dried (0.8 g, 72%).

### Example 236

### 4-(4-{5-[2-(3-Methoxy-phenyl)-vinyl]-pyrimidin-2-ylamino}-2-trifluoromethoxy-benzenesulfonyl)-piperazine-1-carboxylic acid tert-butyl ester (Intermediate 133)

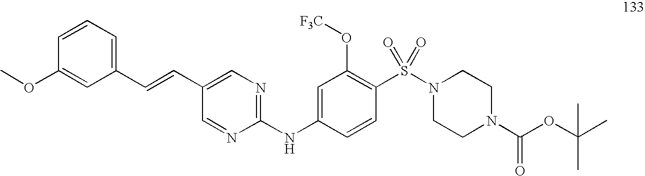

A mixture of intermediate 13 (0.138 g, 0.61 mmol), intermediate 132 (0.36 g, 0.73 mmol), Pd₂(dba)₃ (0.056 g, 0.06 mmol), Xantphos (0.07 g, 0.122 mmol) and cesium carbonate (0.594 g, 1.8 mmol) were suspended in dioxane (10 mL), sealed in a microwave reaction tube and irradiated with microwaves at 160° C. for 15 min. The reaction mixture was cooled to room temperature and centrifuged down. This was then decanted and evaporated to dryness. The resulting residue was used without further purification (0.2 g, 61%).

### Example 237

### 3-(2-{2-[4-(piperazine-1-sulfonyl)-3-trifluoromethoxy-phenylamino]-pyrimidin-5-yl}-vinyl)-phenol (Compound CII)

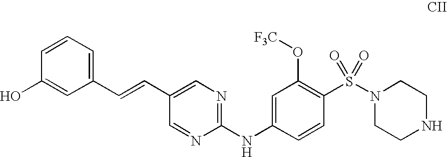

A stirring solution of intermediate 133 (0.2 g, 0.31 mmol) in DCM (10 mL) was treated with BBr₃ (2 mL, 2 mmol). After 2 h, reaction was quenched by careful addition of MeOH (10 mL) and evaporated to dryness. The resulting residue was purified by HPLC. Pure fractions were combined and diluted with EtOAc and neutralized with saturated sodium bicarbonate. Organic phase was isolated, dried over sodium sulfate, filtered and evaporated to afford the title compound. Yellow solids (0.065 g, 40%).

¹H NMR (DMSO-d₆): δ 3.14 (br s, 4H), 3.30-3.32 (m, 4H), 6.72 (dd, J=8.8, 2.9 Hz, 1H), 6.98-7.01 (m, 2H), 7.09 (d, J=16.6 Hz, 1H), 7.18 (t, J=7.9 Hz, 1H), 7.29 (d, J=16.6 Hz, 1H), 7.84 (d, J=8.9 Hz, 1H), 7.94 (dd, J=9.0, 2.1 Hz, 1H), 8.32 (s, 1H), 8.88 (s, 2H), 9.62 (br s, 2H), 10.69 (s, 1H). MS (ES+): m/z 522 (M+H)⁺.

### Example 238

### 4-(4-Bromo-3-trifluoromethyl-benzenesulfonyl)-piperazine-1-carboxylic acid tert-butyl ester (Intermediate 134)

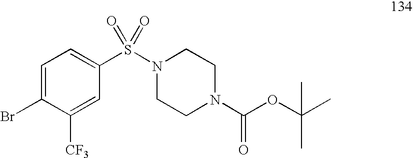

Piperazine-1-carboxylic acid tert-butyl ester (2.1 g, 11.3 mmol) was dissolved in water (40 mL) and treated with 4-bromo-3-trifluoromethyl-benzenesulfonyl chloride (0.73 g, 2.26 mmol). This mixture was stirred vigorously for 18 h. Solids were then filtered off and dried (0.8 g, 75%).

### Example 239

### 4-(4-{5-[2-(3-Methoxy-phenyl)-vinyl]-pyrimidin-2-ylamino}-3-trifluoromethyl-benzenesulfonyl)-piperazine-1-carboxylic acid tert-butyl ester (Intermediate 135)

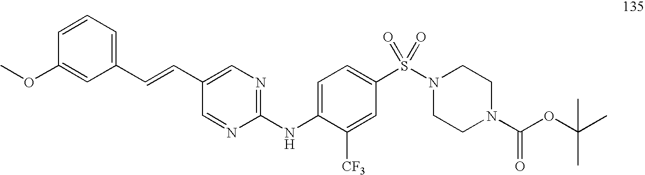

A mixture of intermediate 13 (0.13 g, 0.57 mmol), intermediate 134 (0.33 g, 0.69 mmol), Pd₂(dba)₃ (0.052 g, 0.06 mmol), Xantphos (0.07 g, 0.11 mmol) and cesium carbonate (0.56 g, 1.7 mmol) were suspended in dioxane (10 mL), sealed in a microwave reaction tube and irradiated with microwaves at 160° C. for 15 min. The reaction mixture was cooled to room temperature and centrifuged down. This was then decanted and evaporated to dryness. The resulting residue was used without further purification (0.2 g, 56%).

### Example 240

### 3-(2-{2-[4-(piperazine-1-sulfonyl)-2-trifluoromethyl-phenylamino]-pyrimidin-5-yl}-vinyl)-phenol (Compound CIII)

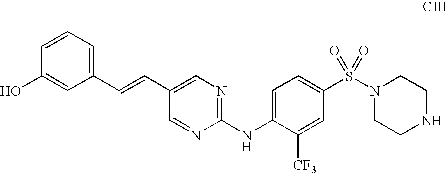

A stirring solution of intermediate 135 (0.2 g, 0.32 mmol) in DCM (10 mL) was treated with BBr₃ (2 mL, 2 mmol). After 2 h, reaction was quenched by careful addition of MeOH (10 mL) and evaporated to dryness. The resulting residue was purified by HPLC. Pure fractions were combined and diluted with EtOAc and neutralized with saturated sodium bicarbonate. Organic phase was isolated, dried over sodium sulfate, filtered and evaporated to afford the title compound. Yellow solids (0.065 g, 40%). %).

¹H NMR (DMSO-d₆): δ 3.15 (br s, 4H), 3.25-3.26 (m, 4H), 6.73 (dd, J=8.8, 2.9 Hz, 1H), 6.98-7.00 (m, 2H), 7.07 (d, J=16.6 Hz, 1H), 7.16 (t, J=7.9 Hz, 1H), 7.24 (d, J=16.6 Hz, 1H), 7.95 (d, J=8.9 Hz, 1H), 8.07 (dd, J=9.0, 2.1 Hz, 1H), 8.29 (d, J=8.8 Hz, 1H), 8.79 (s, 2H), 9.11 (s, H), 9.65 (s, 1H), 9.72 (br s, 2H). MS (ES+): m/z 506 (M+H)⁺.

### Example 241

### 4-(4-Bromo-phenylsulfanylmethyl)-piperidine-1-carboxylic acid tert-butyl ester (Intermediate 136)

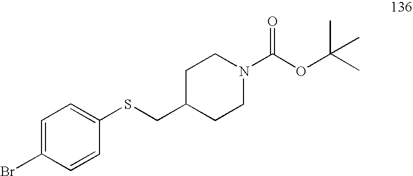

4-Bromomethyl-piperidine-1-carboxylic acid tert-butyl ester (1 g, 3.21 mmol), 4-bromothiophenol (0.527 g, 2.79 mmol) and cesium carbonate (2.27 g, 7 mmol) were mixed in acetone (25 mL) at room temperature. The mixture was heated to reflux and stirred for 3 h. Upon completion, the reaction mixture was filtered and the filtrate was concentrated. The resulting clear oil was used without further purification (1 g, 85%).

### Example 242

### 4-(4-Bromo-benzenesulfonylmethyl)-piperidine-1-carboxylic acid tert-butyl ester (Intermediate 137)

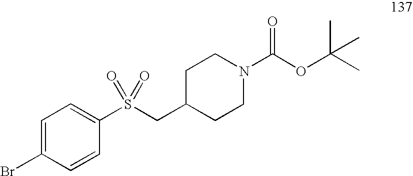

Sodium perborate tetrahydrate (1 g, 7.14 mmol) and intermediate 136 (1 g, 2.38 mmol) and were heated to 55° C. in HOAc and stirred for 18 h. Reaction was cooled to room temperature and poured onto water. Aqueous phase was extracted with EtOAc (3×100 mL). Organic phases were combined and washed carefully with saturated sodium bicarbonate solution (CAUTION: copious gas evolution), dried over sodium sulfate, filtered and evaporated to white solids (1 g, 93%).

### Example 243

### 4-(4-{5-[2-(3-Methoxy-phenyl)-vinyl]-pyrimidin-2-ylamino}-benzenesulfonylmethyl)-piperidine-1-carboxylic acid tert-butyl ester (Intermediate 138)

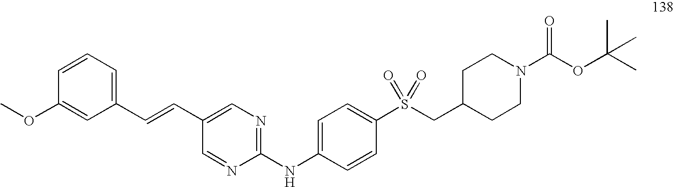

A mixture of intermediate 13 (0.12 g, 0.53 mmol), intermediate 137 (0.287 g, 0.64 mmol), Pd₂(dba)₃ (0.049 g, 0.05 mmol), Xantphos (0.061 g, 0.1 mmol) and cesium carbonate (0.52 g, 1.6 mmol) were suspended in dioxane (10 mL), sealed in a microwave reaction tube and irradiated with microwaves at 160° C. for 15 min. The reaction mixture was cooled to room temperature and centrifuged down. This was then decanted and evaporated to dryness. The resulting residue was used without further purification (0.25 g, 79%).

### Example 244

### 3-(2-{2-[4-(Piperidin-4-ylmethanesulfonyl)-phenylamino]-pyrimidin-5-yl}-vinyl)-phenol (Compound CIV)

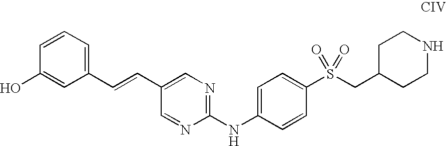

A stirring solution of intermediate 138 (0.25 g, 0.42 mmol) in DCM (10 mL) was treated with BBr₃ (2 mL, 2 mmol). After 5 h, reaction was quenched by careful addition of MeOH (10 mL) and evaporated to dryness. The resulting residue was purified by HPLC. Pure fractions were combined and diluted with EtOAc and neutralized with saturated sodium bicarbonate. Organic phase was isolated, dried over sodium sulfate, filtered and evaporated to afford the title compound. Yellow solids (0.05 g, 27%).

¹H NMR (DMSO-d₆): δ 1.49-1.58 (m, 2H), 1.88-1.91 (m, 2H), 2.08 (br s, 1H), 2.79-2.86 (m, 2H), 3.12 (br s, 2H), 3.27 (d, J=6.4 Hz, 1H), 6.73 (dd, J=8.8, 2.9 Hz, 1H), 6.98-7.00 (m, 2H), 7.07 (d, J=16.6 Hz, 1H), 7.16 (t, J=7.8 Hz, 1H), 7.25 (d, J=16.6 Hz, 1H), 7.81 (d, J=8.7 Hz, 1H), 8.05 (d, J=8.8 Hz, 2H), 8.84 (s, 2H), 9.18 (br s, 1H), 9.36 (br s, 1H), 10.41 (s, 1H). MS (ES+): m/z 451 (M+H)⁺.

### Example 245

### 4-[4-(5-Vinyl-pyrimidin-2-ylamino)-benzenesulfonylmethyl]-piperidine-1-carboxylic acid tert-butyl ester (Intermediate 139)

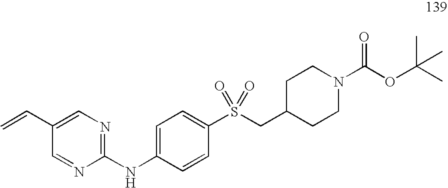

A mixture of intermediate 1 (0.091 g, 0.74 mmol), intermediate 137 (0.367 g, 0.81 mmol), Pd₂(dba)₃ (0.068 g, 0.074 mmol), Xantphos (0.086 g, 0.15 mmol) and cesium carbonate (0.723 g, 2.2 mmol) were suspended in dioxane (10 mL), sealed in a microwave reaction tube and irradiated with microwaves at 160° C. for 15 min. The reaction mixture was cooled to room temperature, diluted with 50 mL DCM and filtered. Filtrate was evaporated to dryness and the resulting residue was purified via silica gel column chromatography. The title intermediate was isolated as beige solids (0.28 g, 77%).

### Example 246

### 4-(4-{5-[2-(2-Amino-benzothiazol-6-yl)-vinyl]-pyrimidin-2-ylamino}-benzenesulfonylmethyl)-piperidine-1-carboxylic acid tert-butyl ester (Intermediate 140)

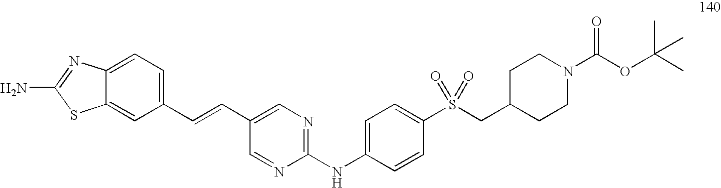

In a dry 15 mL microwave vial were combined 6-bromo-benzothiazol-2-ylamine (0.13 g, 0.56 mmol), intermediate 139 (0.23 g, 0.47 mmol), cesium carbonate (0.61 g, 1.9 mmol), tri-tert-butyl phosphine (1M in toluene) (0.23 mL, 0.23 mmol) and tris(dibenzylideneacetone) dipalladium (0.043 g, 0.047 mmol) were combined. Reactants were diluted with dioxane (6 mL), flushed with argon and irradiated for 15 min at 160° C. Reaction was then spun down, decanted and solvents removed. The residue was then diluted with DCM and adsorbed onto silica gel. Crude product was purified via silica gel column chromatograph, yellow solids (0.1 g, 33% yield). MS (ES+): m/z 641 (M+H)⁺.

### Example 247

### 6-(2-{2-[4-(Piperidin-4-ylmethanesulfonyl)-phenylamino]-pyrimidin-5-yl}-vinyl)-benzothiazol-2-ylamine (Compound CV)

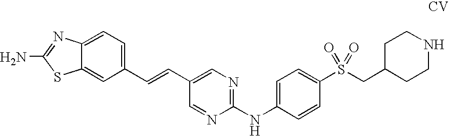

A stirring solution of intermediate 140 (0.1 g, 0.16 mmol) in DCM (10 mL) was treated with BBr₃ (1 mL, 1 mmol). After 4 h, reaction was quenched by careful addition of MeOH (10 mL) and evaporated to dryness. The resulting residue was purified by HPLC. Pure fractions were combined and diluted with EtOAc and neutralized with saturated sodium bicarbonate. Organic phase was isolated, dried over sodium sulfate, filtered and evaporated to afford the title compound. Yellow solids (0.025 g, 32%).

¹H NMR (DMSO-d₆/D₂O): δ 1.11-1.13 (m, 2H), 1.60-1.62 (m, 2H), 1.78 (br s, 1H), 2.31-2.36 (m, 2H), 2.78-2.81 (br s, 2H), 7.06 (d, J=16.6 Hz, 1H), 7.28-7.33 (m, 2H), 7.46 (dd, J=8.4, 1.6 Hz, 1H), 7.78 (d, J=8.7 Hz, 1H), 7.88 (s, 1H), 8.01 (d, J=8.8 Hz, 2H), 8.79 (s, 2H). MS (ES+): m/z 507 (M+H)⁺.

### Example 248

### 4-(Toluene-4-sulfonyloxy)-azepane-1-carboxylic acid tert-butyl ester (Intermediate 141)

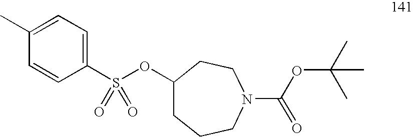

A mixture of 4-hydroxy-azepane-1-carboxylic acid tert-butyl ester (3.9 g, 18.14 mmol) and 4-dimethylaminopyridine (0.1 g) were diluted with DCM (30 mL) and triethylamine (6.3 mL, 45.35 mmol). Resulting mixture was chilled to 0° C. and treated dropwise with a solution of 4-methyl-benzenesulfonyl chloride (4.16 g, 21.8 mmol) in 10 mL of DCM. Reaction was then allowed to come to room temperature and stir overnight. After 16 h, now brown reaction mixture was poured onto water and washed once. Organic phase was evaporated to clear oil that was used without further purified (6.7 g, 100%).

### Example 249

### 4-(4-Bromo-phenylsulfanyl)-azepane-1-carboxylic acid tert-butyl ester (Intermediate 142)

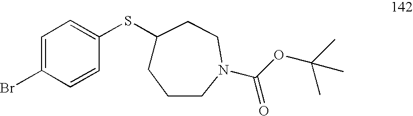

A mixture of 4-bromothiophenol (4.12 g, 21.8 mmol), intermediate 141 (6.7 g, 18.2 mmol) and cesium carbonate (14.8 g, 45.4 mmol) were mixed in acetone (75 mL) at room temperature. The mixture was heated to reflux and stirred for 3 h. Upon completion, the reaction mixture was filtered and the filtrate was concentrated. The resulting brown oil was purified via silica gel column chromatography. Eluting with 100% DCM afforded the title intermediate as clear oil (4.5 g, 64%).

### Example 250

### 4-(4-Bromo-benzenesulfonyl)-azepane-1-carboxylic acid tert-butyl ester (Intermediate 143)

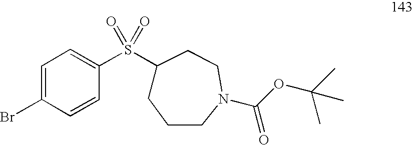

Sodium perborate tetrahydrate (5.4 g, 34.8 mmol) and intermediate 45 (4.47 g, 11.6 mmol) and were heated to 60° C. in HOAc and stirred for 3 h. Reaction solvents then removed. Residue was diluted with EtOAc (200 mL) and washed with water. Organic phase washed with brine, dried over sodium sulfate, filtered and evaporated to viscous oil that was used without further purification (5 g, 100%).

### Example 251

### 4-[4-(5-Vinyl-pyrimidin-2-ylamino)-benzenesulfonyl]-azepane-1-carboxylic acid tert-butyl ester (Intermediate 144)

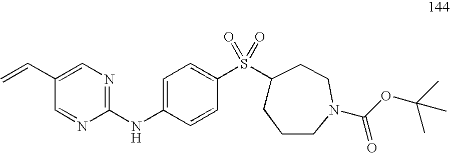

A mixture of intermediate 1 (0.128 g, 1.04 mmol), intermediate 143 (0.5 g, 0.81 mmol), Pd₂(dba)₃ (0.095 g, 0.104 mmol), Xantphos (0.12 g, 0.21 mmol) and cesium carbonate (0.99 g, 3.12 mmol) were suspended in dioxane (18 mL), sealed in a microwave reaction tube and irradiated with microwaves at 160° C. for 15 min. The reaction mixture was cooled to room temperature, diluted with 50 mL DCM and filtered. Filtrate was evaporated to dryness and the resulting residue was purified via silica gel column chromatography. The title intermediate was isolated as white solids (0.28 g, 58%).

### Example 252

### 4-(4-{5-[2-(2-Amino-benzothiazol-6-yl)-vinyl]-pyrimidin-2-ylamino}-benzenesulfonyl)-azepane-1-carboxylic acid tert-butyl ester (Intermediate 145)

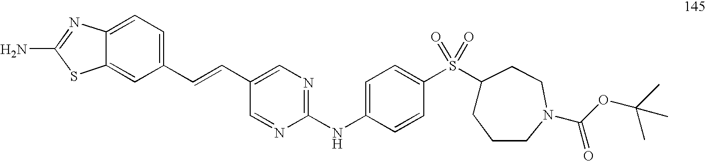

A mixture of intermediate 144 (0.179 g, 0.4 mmol), 6-bromo-benzothiazol-2-ylamine (0.179 g, 0.8 mmol), Pd(OAc)₂ (0.0088 g, 0.04 mmol) and triethylamine (0.35 mL, 1.95 mmol) were suspended in DMF (15 mL), sealed in a microwave reaction tube and irradiated with microwaves at 180° C. for 30 min. The reaction mixture was cooled to room temperature and centrifuged down. The reaction was then decanted and the organic phase concentrated in vacuo. The residue was purified by HPLC. The title intermediate was isolated as yellow solids (0.125 g, 53%).

### Example 253

### 6-(2-{2-[4-(Azepane-4-sulfonyl)-phenylamino]-pyrimidin-5-yl}-vinyl)-benzothiazol-2-ylamine (Compound CVI)

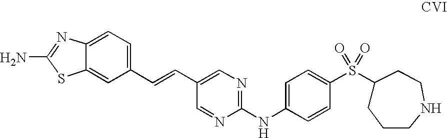

A stirring solution of intermediate 145 (0.125 g, 0.21 mmol) in DCM (10 mL) was treated with TFA (0.5 mL) and allowed to stir at room temperature for 2 h. Solvents were then removed and the resulting residue was purified by HPLC. The title compound was isolated as white solids (0.01 g, 10%).

¹H NMR (DMSO-d₆): δ 1.42 (br s, 2H), 1.50-1.59 (m, 2H), 1.71 (br s, 1H), 2.01-2.05 (m, 2H), 2.54-2.59 (m, 1H), 2.68-2.79 (m, 5H), 7.07 (d, J=16.6 Hz, 1H), 7.30-7.34 (m, 2H), 7.44 (dd, J=8.4, 1.7 Hz, 1H), 7.58 (br s, 2H), 7.75 (d, J=8.9 Hz, 2H), 7.88 (d, J=1.7 Hz, 1H), 7.95 (s, 1H), 8.65 (m, 1H), 8.03 (d, J=8.9 Hz, 1H), 8.81 (s, 2H), 10.35 (s, 1H). MS (ES+): m/z 507 (M+H)⁺.

### Example 254

### 4-(4-{5-[2-(1-Oxo-indan-5-yl)-vinyl]-pyrimidin-2-ylamino}-benzenesulfonyl)-piperidine-1-carboxylic acid tert-butyl ester (Intermediate 146)

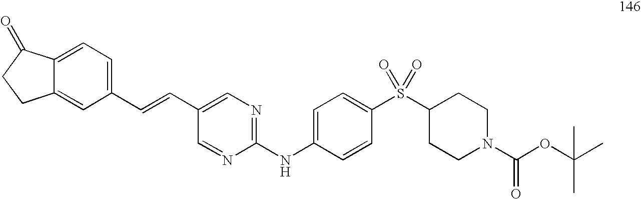

A mixture of intermediate 6 (0.5 g, 1.13 mmol), 5-bromo-indan-1-one (0.285 g, 1.35 mmol), Pd(OAc)₂ (0.025 g, 0.04 mmol) and triethylamine (0.77 mL, 5.6 mmol) were suspended in DMF (15 mL), sealed in a microwave reaction tube and irradiated with microwaves at 180° C. for 55 min. The reaction mixture was cooled to room temperature and centrifuged down. The reaction was then decanted and the organic phase concentrated in vacuo. The residue was purified by silica gel column chromatography. The title intermediate was isolated as white solids (0.136 g, 21%).

### Example 255

### 5-(2-{2-[4-(Piperidine-4-sulfonyl)-phenylamino]-pyrimidin-5-yl}-vinyl)-indan-1-one oxime (Compound CVII)

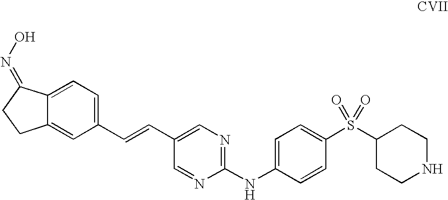

A solution of intermediate 146 (0.136 g, 0.24 mmol) in pyridine (5 mL) was treated with hydroxylamine hydrochloride (0.1 g, 1.42 mmol). This was then heated to 50° C. for 5 h. Reaction was then cooled to room temperature and solvents removed. The resulting residue was treated with 15 mL of 20% TFA in DCM followed by removal of all solvents. The resulting residue was purified via HPLC to provide the title compound as yellow solids (0.1 g, 86%).

¹H NMR (DMSO-d₆): δ 1.31 (br s, 2H), 1.68-1.71 (m, 2H), 2.36-2.39 (m, 2H), 2.79-2.82 (m, 2H), 2.94 (br s, 1H), 3.00-3.03 (m, 2H), 3.15-3.17 (br s, 2H), 4.15 (br s, 1H), 6.23 (d, J=16.6 Hz, 1H), 7.37 (d, J=16.6 Hz, 1H), 7.47 (d, J=8.2 Hz, 1H), 7.56 (d, J=10.5 Hz, 2H), 7.72 (d, J=8.9 Hz, 2H), 8.04 (d, J=8.9 Hz, 2H), 8.85 (s, 2H), 10.40 (s, 1H). MS (ES+): m/z 490 (M+H)⁺.

### Example 256

### 4-(4-{5-[2-(1-Oxo-indan-4-yl)-vinyl]-pyrimidin-2-ylamino}-benzenesulfonyl)-piperidine-1-carboxylic acid tert-butyl ester (Intermediate 147)

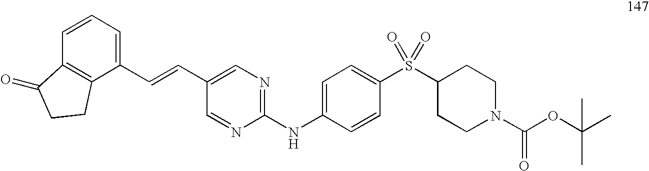

A mixture of intermediate 6 (0.5 g, 1.13 mmol), 5-bromo-indan-1-one (0.285 g, 1.35 mmol), Pd(OAc)₂ (0.025 g, 0.04 mmol) and triethylamine (0.77 mL, 5.6 mmol) were suspended in DMF (15 mL), sealed in a microwave reaction tube and irradiated with microwaves at 180° C. for 55 min. The reaction mixture was cooled to room temperature and centrifuged down. The reaction was then decanted and the organic phase concentrated in vacuo. The residue was purified by silica gel column chromatography. The title intermediate was isolated as white solids (0.060 g, 10%).

### Example 257

### 4-(2-{2-[4-(Piperidine-4-sulfonyl)-phenylamino]-pyrimidin-5-yl}-vinyl)-indan-1-one oxime (Compound CVIII)

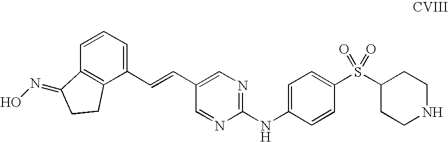

A solution of intermediate 147 (0.05 g, 0.087 mmol) in pyridine (5 mL) was treated with hydroxylamine hydrochloride (0.036 g, 0.52 mmol). This was then heated to 50° C. for 13 h. Reaction was then cooled to room temperature and solvents removed. The resulting residue was treated with 15 mL of 20% TFA in DCM followed by removal of all solvents. The resulting residue was purified via HPLC to provide the title compound as yellow solids (0.01 g, 23%).

¹H NMR (DMSO-d₆): δ 1.22 (br s, 2H), 1.36-1.43 (m, 2H), 1.78-1.81 (m, 2H), 2.83-2.86 (m, 2H), 3.02-3.04 (m, 2H), 3.15-3.22 (m, 5H), 7.19 (d, J=16.6 Hz, 1H), 7.32 (d, J=7.7 Hz, 1H), 7.38 (d, J=16.6 Hz, 1H), 7.48 (d, J=8.2 Hz, 1H), 7.68 (d, J=10.5 Hz, 2H), 7.73 (d, J=8.9 Hz, 2H), 8.05 (d, J=8.9 Hz, 2H), 8.90 (s, 2H), 10.43(s, 1H), 10.89 (br s, 1H). MS (ES+): m/z 490 (M+H)⁺.

### Example 258

### 4-(4-{5-[2-(2-Amino-benzothiazol-6-yl)-vinyl]-pyrimidin-2-ylamino}-benzenesulfonyl)-piperidine-1-carboxylic acid ethylamide (Compound CIX)

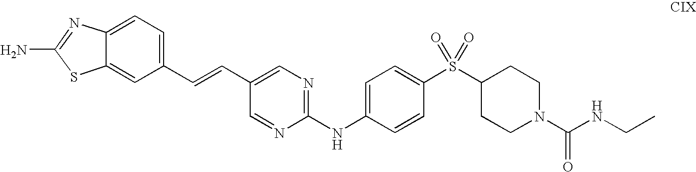

A slurry of 6-(2-{2-[4-(piperidine-4-sulfonyl)-phenylamino]-pyrimidin-5-yl}-vinyl)-benzothiazol-2-ylamine (0.1 g, 0.189 mmol) was diluted with triethylamine (0.066 mL, 0.47 mmol) and treated with 2 drops of ethyl isocyanate (0.013 g, 0.189 mmol). After 5 h, yellow reaction solids were filtered off, washed with water and dried (0.065 g, 61%).

¹H NMR (DMSO-d₆): δ 0.96 (t, J=7.2 Hz, 1H), 1.26-1.34 (m, 2H), 1.77-1.78 (m, 2H), 2.60-2.65 (m, 2H), 2.97-3.02 (m, 2H), 3.99-4.01 (m, 2H), 6.47 (t, J=5.3 Hz, 1H), 7.07 (d, J=16.6 Hz, 1H), 7.30-7.33 (m, 2H), 7.44 (dd, J=8.5, 1.5 Hz, 1H), 7.58 (s, 2H), 7.72 (d, J=8.2 Hz, 1H), 7.88 (d, J=1.1 Hz, 2H), 8.05 (d, J=8.9 Hz, 2H), 8.81 (s, 2H), 10.37(s, 1H). MS (ES+): m/z 564 (M+H)⁺.

### Example 259

### 4-(4-{5-[2-(2-Amino-benzothiazol-6-yl)-vinyl]-pyrimidin-2-ylamino}-benzoyl)-piperazine-1-carboxylic acid tert-butyl ester (Intermediate 148)

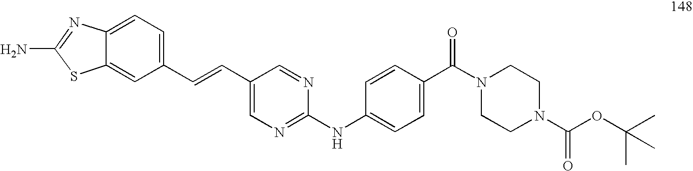

Argon was bubbled in a reaction vial containing intermediate 86 (0.818 mg, 2 mmol), 6-Bromo-benzothiazol-2-ylamine (0.916 mg, 4 mmol), Pd₂(dba)₃ (0.458 mg, 0.5 eq), tri-tert-butyl-phosphane (0.404 mg, 2.0 mmol), cesium carbonate (1.30 g, 4 mmol) in dioxane (20 mL) for 10 min and sealed. The reaction mixture was heated at 160° C. for 6 h in a microwave reactor. The material was filtered and purified using flash chromatography (SiO2, 10% ethyl acetate in hexanes to 100% ethyl acetate over 15 min) to afford the title intermediate (0.25 g, 22%) as a yellow solid. MS (ES+): m/z 558 (M+H)⁺.

### Example 260

### (4-{5-[2-(2-Amino-benzothiazol-6-yl)-vinyl]-pyrimidin-2-ylamino}-phenyl)-piperazin-1-yl-methanone (Compound CX)

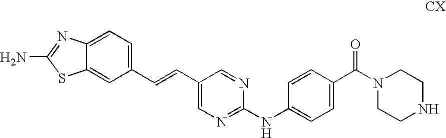

A solution of 148 (0.2 g, 0.36 mmol), and hydrogen chloride in dioxane (4M solution, 1 mL) in ethanol was stirred at 60° C. for 2 h. The reaction mixture was concentrated and purified by high performance liquid chromatography (C-18 column, water-acetonitrile). The fractions from HPLC were combined and poured into saturated NaHCO₃ solution (10 mL). The aqueous layer was extracted with EtOAc (3×30 mL) and the combined organic layers were washed with brine, dried over anhydrous Na₂SO₄ and filtered. The filtrate was concentrated to afford the title compound (0.051 g, 31%) as a yellow solid.

¹H NMR (500 MHz, DMSO-d₆): δ 2.69 (br s, 4H), 3.32-3.5 (m, 5H), 3.3 (br s, 2H), 7.05 (d, J=16.5 Hz, 1H), 7.27 (d, J=16.6 Hz, 1H), 7.20-7.35 (m, 3H), 7.51-7.60 (m, 2H), 7.84-7.88 (m, 2H), 8.75 (s, 2H), 9.99 (s, 1H). MS (ES+): m/z 458 (M+H)⁺.

### Example 261

### {5-[2-(3-Methoxy-phenyl)-vinyl]-pyrimidin-2-yl}-[4-(piperidine-4-sulfonyl)-phenyl]-amine (Compound CXI)

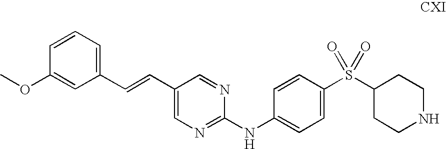

A solution of 4-(4-{5-[2-(3-methoxy-phenyl)-vinyl]-pyrimidin-2-ylamino}-benzenesulfonyl)-piperidine-1-carboxylic acid tert-butyl ester (0.195 g, 0.35 mmol) and hydrogen chloride solution (4M solution in dioxane, 1 mL) in dichloromethane (5 mL) was stirred at room temperature for 30 min. The solution was concentrated under reduced pressure to remove the solvent, the residue was dissolved in dimethyl formamide and purified on HPLC. The fractions from HPLC were combined and poured into saturated NaHCO₃ solution (10 mL).

The aqueous layer was extracted with dichloromethane (3×30 mL) and the combined organic layers were washed with brine, dried over anhydrous Na₂SO₄ and filtered. The filtrate was concentrated, treated with 1 mL of 4 M solution of hydrogen chloride in dioxane. The solution was concentrated under reduced pressure to afford the title compound (0.091 g, 53%) as hydrochloride salt.

¹H NMR (500 MHz, DMSO-d₆): δ 1.60-1.80 (m, 2H), 2.01 (d, 12.7 Hz, 2H), 2.80-2.89 (m, 2H), 3.31 (d, J=11.7 Hz, 2H), 3.45-3.55 (m, 1H), 3.80 (s, 3H), 6.87 (d, J=7.1 Hz, 1H), 7.10-7.42 (m, 4H), 7.75 (d, J=8.9 Hz, 2H), 8.09 (d, J=8.9 Hz, 2H), 8.69 (d, J=11.4 Hz, 1H), 8.85 (s, 2H), 9.29 (d, J=10.5 Hz, 1H), 10.46 (s, 1H). MS (ES+): m/z 451 (M+H)⁺.

### Example 262

### 4-[2-(4-Bromo-phenylsulfanyl)-ethyl]-morpholine (Intermediate 149)

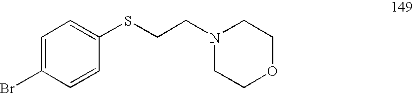

A solution of 4-bromo-benzenethiol (10 g, 53 mmol), 4-(2-chloro-ethyl)-morpholine (9.9 g, 53 mmol) and cesium carbonate (34.6 g, 106 mmol) in acetonitrile was stirred at room temperature for 4 days. The reaction mixture was filtered and the solid was washed with ethyl acetate (2×50 mL). Organic layers were combined, concentrated and purified using flash chromatography (SiO₂, 5% ethyl acetate in hexanes to 100% ethyl acetate over 15 min) to afford the title intermediate (14.1 g, 88%) as a tan colored solid. MS (ES+): m/z 304/302 (M+H)⁺.

### Example 263

### [4-(2-Morpholin-4-yl-ethylsulfanyl)-phenyl]-(5-vinyl-pyrimidin-2-yl)-amine (Intermediate 150)

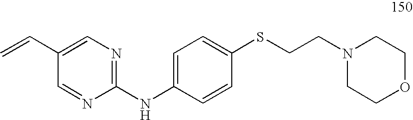

Argon was bubbled in to a reaction mixture containing intermediate 1 (0.238 g, 2 mmol), intermediate 149 (0.604 g, 2 mmol), Pd₂(dba)₃ (0.092 g, 0.1 mmol), xantphos (0.174 mg, 0.3 mmol), cesium carbonate (0.13 g, 0.4 mmol) in dioxane (20 mL) for 10 min. The reaction mixture was sealed and heated in a microwave reaction at 160° C. for 4 h. The reaction mixture was cooled to room temperature, filtered, concentrated and purified on a flash chromatography (SiO2, 10% ethyl acetate in hexanes to 100% ethyl acetate over 15 min) to afford the title intermediate (0.0116 g, 34%) as a faint yellow solid. MS (ES+): m/z 343 (M+H)⁺.

### Example 264

### 6-(2-{2-[4-(2-Morpholin-4-yl-ethylsulfanyl)-phenylamino]-pyrimidin-5-yl}-vinyl)-benzothiazol-2-ylamine (Compound CXII)

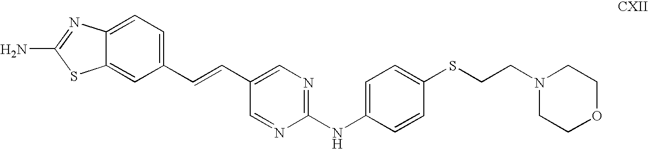

Argon was bubbled in to a reaction mixture containing intermediate 150 (0.338 g, 0.99 mmol), 6-bromo-benzothiazol-2-ylamine (0.275 g, 1.2 mmol), Pd₂(dba)₃ (0.11 g, 0.12 mmol), tri-tert-butyl-phosphane (0.073 g, 0.36 mmol), cesium carbonate (0.196 g, 0.6 mmol) in dioxane (10 mL) for 10 min. The reaction vessel was sealed and heated in a microwave at 160° C. for 30 min. The reaction mixture was filtered, concentrated and purified using a flash chromatography (10% ethyl acetate in hexanes to 100% ethyl acetate) to afford the title compound (0.37 g, 76%) as a yellow solid.

¹H NMR (500 MHz, DMSO-d₆): δ 2.37 (br s, 4H), 2.48 (t, J=8.7 Hz, 2H), 3.00 (t, J=7.7 Hz, 2H), 3.54 (t, J=4.5 Hz, 4H), 7.03 (d, J=16.5 Hz, 1H), 7.25 (d, J=16.5 Hz, 1H), 7.32 (d, J=8.8 Hz, 2H), 7.33 (s, 1H), 7.42 (dd, J=8.4, 1.5 Hz, 1H), 7.56 (s, 2H), 7.75 (d, J=8.7 Hz, 2H), 7.86 (d, J=1.4 Hz, 1H), 8.71 (s, 2H), 9.83 (s, 1H). MS (ES+): m/z 491 (M+H)⁺.

### Example 265

### 6-(2-{2-[4-(2-Morpholin-4-yl-ethanesulfinyl)-phenylamino]-pyrimidin-5-yl}-vinyl)-benzothiazol-2-ylamine (Compound CXIII)

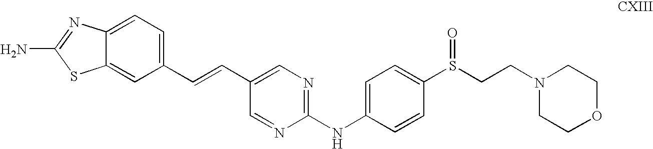

A solution of the above-described compound CXII (0.168 g, 0.34 mmol), sodium perborate hydrate (0.052 g, 0.34 mmol) in acetic acid (3.4 mL) was stirred at room temperature for 2 h. The reaction mixture was concentrated under reduced pressure, dissolved in dimethyl formamide and purified on high performance liquid chromatography (C18 column, water-acetonitrile). Appropriate fractions were collected, concentrated under reduced pressure, dissolved in methanol and passed through a resin column to remove trifluoroacetic acid. The eluent was concentrated to give the title compound (0.072 g, 42%) as a yellow solid.

¹H NMR (500 MHz, DMSO-d₆): δ 3.02 (br s, 2H), 3.33 (br s, 6H). 3.59 (br s, 4H), 7.06 (d, J=16.5 Hz, 1H), 7.29 (d, J=16.6 Hz, 1H), 7.33 (d, J=8.3 Hz, 1H), 7.43 (dd, J=8.6, 1.6 Hz, 1H), 7.58 (s, 2H), 7.61 (d, J=8.8 Hz, 2H), 7.87 (d, J=1.6 Hz, 1H), 8.00 (d, J=8.8 Hz, 2H), 8.77 (s, 2H), 10.13 (s, 1H). MS (ES+): m/z 507 (M+H)⁺.

### Example 266

### 5-[(E)-2-(1H-Indol-4-yl)vinyl]-N-[4-(2-pyrrolidin-1-ylethoxy)phenyl]pyrimidin-2-amine (Compound CXIV)

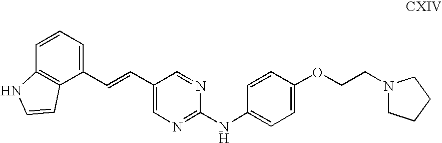

2-5 mL Emrys microwave vial was charged with the intermediate 11 (94.5 mg, 0.4 mmol), 1-[2-(4-bromophenoxy)ethyl]pyrrolidine (110.0 mg, 0.4 mmol), Pd(OAc)₂ (9.0 mg, 0.04 mmol), Xantphos (35.0 mg, 0.06 mmol), cesium carbonate (196.0 g, 0.6 mmol) and anhydrous dioxane (5 mL). The mixture was purged with argon gas for 10 min, then sealed and irradiated in a microwave (Initiator, Biotage) at 160° C. for 30 min. After cooling to room temperature, the cap was removed and the resulting mixture was concentrated in vacuo. The residue was re-dissolved in 5 mL of DMF, filtered though 0.2 micron syringe filter and purified by reverse-phase preparative HPLC in CH₃CN/H₂O system containing 0.05% of TFA. Fractions containing the product were combined and poured into EtOAc (50 mL). The solution was treated with saturated aqueous NaHCO₃ (2×30 mL), washed with brine (2×30 mL), dried over anhydrous Na₂SO₄ and filtered. The filtrate was concentrated in vacuo to give the title compound as a yellow solid (31.6 mg, 19%).

¹H NMR (500 MHz, DMSO-d₆): δ 1.68-1.78 (m, 4H), 2.06 (s, 3H), 2.62-2.78 (m, 4H), 2.88-2.98 (m, 2H), 4.09 (t, J=5.5 Hz, 2H), 6.90-6.92 (m, 3H), 7.10 (t, J=7.7 Hz, 1H), 7.20 (d, J=16.6 Hz, 1H), 7.29 (d, J=7.3 Hz, 1H), 7.32 (d, J=8.1 Hz, 1H), 7.42 (t, J=2.7 Hz, 1H), 7.58 (d, J=16.7 Hz, 1H), 7.67 (d, J=9.1 Hz, 1H), 8.79 (s, 2H), 9.60 (s, 1H), 11.20 (s, 1H). MS (ES+): m/z 426 (M+H)⁺.

### Example 267

### 4-({5-[(E)-2-(1H-Indol-4-yl)vinyl]pyrimidin-2-yl}amino)-N-[4-(2-pyrrolidin-1-ylethyl)benzenesulfonamide (Compound CXV)

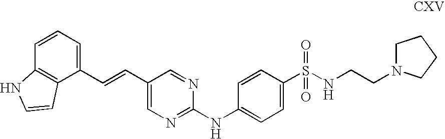

2-5 mL Emrys microwave vial was charged with the intermediate 11 (94.5 mg, 0.4 mmol), intermediate 95 (146.6 mg, 0.4 mmol), Pd(OAc)₂ (9.0 mg, 0.04 mmol), Xantphos (35 mg, 0.06 mmol), cesium carbonate (196.0 g, 0.6 mmol) and anhydrous dioxane (5 mL). The mixture was purged with argon gas for 10 min, then sealed and irradiated in a microwave (Initiator, Biotage) at 160° C. for 30 min. After cooling to room temperature, the cap was removed and the resulting mixture was concentrated in vacuo. The residue was re-dissolved in 5 mL of DMF, filtered though 0.2 micron syringe filter and purified by reverse-phase preparative HPLC in CH₃CN/H₂O system containing 0.05% of TFA. Fractions containing the product were combined and poured into EtOAc (50 mL). The solution was treated with saturated aqueous NaHCO₃ (2×30 mL), washed with brine (2×30 mL), dried over anhydrous Na₂SO₄ and filtered. The filtrate was concentrated in vacuo to give the title compound as a bright-yellow solid (97.3 mg, 45%).

¹H NMR (500 MHz, DMSO-d₆): δ 1.62-1.68 (m, 4H), 2.41-2.48 (m, 4H), 2.84 (t, J=6.6 Hz, 2H), 3.34 (m, 2H), 6.94 (br s, 1H), 7.11 (t, J=7.7 Hz, 1H), 7.26 (d, J=16.6 Hz, 1H), 7.32 (d, J=7.34 Hz, 1H), 7.35 (d, J=8.05 Hz, 1H), 7.38 (br s, 1H), 7.43 (t, J=2.7 Hz, 1H), 7.68 (d, J=16.6 Hz, 1H), 7.73 (d, J=8.8 Hz, 2H), 7.99 (d, J=8.8 Hz, 2H), 8.93 (s, 2H), 10.25 (s, 1H), 11.20 (s, 1H)

MS (ES+): m/z 489 (M+H)⁺

### Example 268

### 5-[(E)-2-(1H-Indol-4-yl)vinyl]-N-{4-[(4-methylpiperazin-1-yl)carbonyl]phenyl}pyrimidin-2-amine (Compound CXVI)

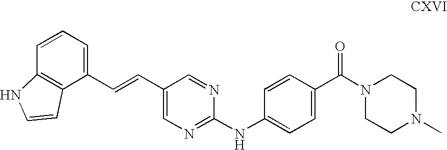

2-5 mL Emrys microwave vial was charged with the intermediate 11 (94.5 mg, 0.4 mmol), 1-(4-bromobenzoyl)-4-methylpiperazine (124.6 mg, 0.44 mmol), Pd(OAc)₂ (9.0 mg, 0.04 mmol), Xantphos (35 mg, 0.06 mmol), cesium carbonate (196.0 g, 0.6 mmol) and anhydrous dioxane (5 mL). The mixture was purged with argon gas for 10 min, then sealed and irradiated in a microwave (Initiator, Biotage) at 160° C. for 30 min. After cooling to room temperature, the cap was removed and the resulting mixture was concentrated in vacuo. The residue was re-dissolved in 5 mL of DMF, filtered though 0.2 micron syringe filter and purified by reverse-phase preparative HPLC in CH₃CN/H₂O system containing 0.05% of TFA. Fractions containing the product were combined and poured into EtOAc (50 mL). The solution was treated with saturated aqueous NaHCO₃ (2×30 mL), washed with brine (2×30 mL), dried over anhydrous Na₂SO₄ and filtered. The filtrate was concentrated in vacuo to give the title compound as a yellow solid (61.2 mg, 35%).

¹H NMR (500 MHz, DMSO-d₆): δ 2.20 (s, 3H), 2.30-2.36 (m, 4H), 3.46-3.52 (m, 4H), 6.91 (m, 1H), 7.10 (t, J=7.2 Hz, 1H), 7.22 (d, J=16.6 Hz, 1H), 7.29 (d, J=7.3 Hz, 1H), 7.32 (d, J=7.3 Hz, 1H), 7.34 (d, J=8.7 Hz, 2H), 7.41 (t, J=2.8 Hz, 1H), 7.63 (d, J=16.6 Hz, 1H), 7.85 (d, J=8.7 Hz, 2H), 8.89 (s, 2H), 10.02 (s, 1H), 11.19 (s, 1H). MS (ES+): m/z 439 (M+H)⁺.

### Example 269

### 5-[(E)-2-(1H-Indol-4-yl)vinyl]-N-{4-[(3-pyrrolidin-1-ylpropyl)sulfonyl]phenyl}pyrimidin-2-amine (Compound CXVII)

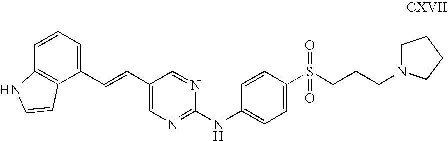

2-5 mL Emrys microwave vial was charged with the intermediate 11 (94.5 mg, 0.4 mmol), 1-{3-[(4-bromophenyl)sulfonyl]propyl}pyrrolidine (146.2 mg, 0.44 mmol), Pd(OAc)₂ (9.0 mg, 0.04 mmol), Xantphos (35 mg, 0.06 mmol), cesium carbonate (196.0 g, 0.6 mmol) and anhydrous dioxane (5 mL). The mixture was purged with argon gas for 10 min, then sealed and irradiated in a microwave (Initiator, Biotage) at 160° C. for 30 min. After cooling to room temperature, the cap was removed and the resulting mixture was concentrated in vacuo. The residue was re-dissolved in 5 mL of DMF, filtered though 0.2 micron syringe filter and purified by reverse-phase preparative HPLC in CH₃CN/H₂O system containing 0.05% of TFA. Fractions containing the product were combined and poured into EtOAc (50 mL). The solution was treated with saturated aqueous NaHCO₃ (2×30 mL), washed with brine (2×30 mL), dried over anhydrous Na₂SO₄ and filtered. The filtrate was concentrated in vacuo to give the title compound as a bright-yellow solid (113.8 mg, 58%).

¹H NMR (500 MHz, DMSO-d₆): δ 1.62-1.68 (m, 4H), 1.74 (m, 2H), 2.50-2.59 (m, 4H), 3.25-3.31 (m, 4H), 6.91-6.93 (m, 1H), 7.10 (t, J=7.7 Hz, 1H), 7.25 (d, J=16.6 Hz, 1H), 7.30 (d, J=7.3 Hz, 1H), 7.34 (d, J=7.9 Hz, 1H), 7.42 (t, J=2.7 Hz, 1H), 7.68 (d, J=16.7 Hz, 1H), 7.80 (d, J=8.9 Hz, 2H), 8.05 (d, J=8.9 Hz, 2H), 8.95 (s, 2H), 10.37 (s, 1H), 11.21 (s, 1H). MS (ES+): m/z 488 (M+H)⁺.

### Example 270

### 5-[(E)-2-(1H-Indol-4-yl)vinyl]-N-{3-[(4-methylpiperazin-1-yl)sulfonyl]phenyl}pyrimidin-2-amine (Compound CXVIII)

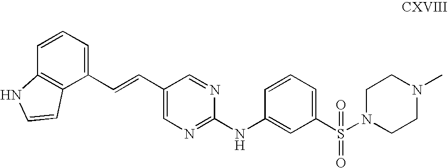

2-5 mL Emrys microwave vial was charged with the intermediate 11 (94.5 mg, 0.4 mmol), 1-{(3-bromophenyl)sulfonyl]-4-methylpiperazine (140.4 mg, 0.44 mmol), Pd(OAc)₂ (9.0 mg, 0.04 mmol), Xantphos (35 mg, 0.06 mmol), cesium carbonate (196.0 g, 0.6 mmol) and anhydrous dioxane (5 mL). The mixture was purged with argon gas for 10 min, then sealed and irradiated in a microwave (Initiator, Biotage) at 160° C. for 30 min. After cooling to room temperature, the cap was removed and the resulting mixture was concentrated in vacuo. The residue was re-dissolved in 5 mL of DMF, filtered though 0.2 micron syringe filter and purified by reverse-phase preparative HPLC in CH₃CN/H₂O system containing 0.05% of TFA. Fractions containing the product were combined and poured into EtOAc (50 mL). The solution was treated with saturated aqueous NaHCO₃ (2×30 mL), washed with brine (2×30 mL), dried over anhydrous Na₂SO₄ and filtered. The filtrate was concentrated in vacuo to give the title compound as a bright-yellow solid (97.0 mg, 51%).

¹H NMR (500 MHz, DMSO-d₆): δ 2.14 (s, 3H), 2.36-2.40 (m, 4H), 2.91-2.95 (m, 4H), 6.91-6.93 (m, 1H), 7.10 (t, J=7.7 Hz, 1H), 7.23 (d, J=16.6 Hz, 1H), 7.26-7.28 (m, 1H), 7.29 (d, J=7.4 Hz, 1H), 7.33 (d, J=7.9 Hz, 1H), 7.42 (t, J=2.7 Hz, 1H), 7.55 (t, J=7.9 Hz, 1H), 7.66 (d, J=16.7 Hz, 1H), 8.02 (ddd, J=8.2, 1.59, 0.75 Hz, 1H), 8.33 (t, J=1.9 Hz, 2H), 8.91 (s, 2H), 10.17 (s, 1H), 11.19 (s, 1H). MS (ES+): m/z 475 (M+H)⁺.

### Example 271

### 5-[(E)-2-(1H-Indol-4-yl)vinyl]-N-[4-(piperazin-1-ylsulfonyl)phenyl]pyrimidin-2-amine (Compound CXIX)

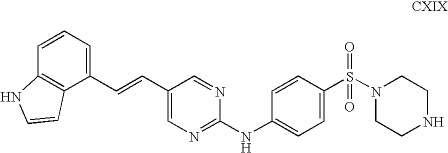

2-5 mL Emrys microwave vial was charged with the intermediate 11 (94.5 mg, 0.4 mmol), 4-[(4-bromophenyl)sulfonyl]piperazine-1-carboxylic acid tert-butyl ester (178.3 mg, 0.44 mmol), Pd(OAc)₂ (9.0 mg, 0.04 mmol), Xantphos (35 mg, 0.06 mmol), cesium carbonate (196.0 g, 0.6 mmol) and anhydrous dioxane (5 mL). The mixture was purged with argon gas for 10 min, then sealed and irradiated in a microwave (Initiator, Biotage) at 160° C. for 30 min. After cooling to room temperature, the cap was removed and the reaction mixture was poured into ca. 100 mL of DCM. The resulting solution was washed with dilute aqueous HCl (2×100 mL), brine (2×100 mL), dried over anhydrous Na₂SO₄, filtered and concentrated in vacuo. The residue was treated with 50% TFA in DCM for 1 h. 5 mL of CH₃CN were added, the reaction mixture was concentrated in vacuo to the total volume of 5 mL, filtered though 0.2 micron syringe filter and purified by reverse-phase preparative HPLC in CH₃CN/H₂O system containing 0.05% TFA. Fractions containing the product were combined and poured into EtOAc (50 mL). The solution was treated with saturated aqueous NaHCO₃ (2×30 mL), washed with brine (2×30 mL), dried over anhydrous Na₂SO₄ and filtered. The filtrate was concentrated in vacuo to give the title compound as a yellow solid (32.3 mg, 17%).

¹H NMR (500 MHz, DMSO-d₆): δ 2.72-2.74 (m, 4H), 2.75-2.78 (m, 4H), 6.91-6.93 (m, 1H), 7.10 (t, J=7.7 Hz, 1H), 7.25 (d, J=16.6 Hz, 1H), 7.30 (d, J=7.24 Hz, 1H), 7.33 (d, J=8.0 Hz, 1H), 7.42 (t, J=2.8 Hz, 1H), 7.63 (d, J=8.9 Hz, 2H), 7.67 (d, J=16.6 Hz, 1H), 8.04 (d, J=8.9 Hz, 2H), 8.95 (s, 2H), 10.34 (s, 1H), 11.23 (s, 1H). MS (ES+): m/z 461 (M+H)⁺.

### Example 272

### 5-[(E)-2-(1H-Indol-4-yl)vinyl]-N-{3-(piperazin-1-ylsulfonyl)phenyl}pyrimidin-2-amine (Compound CXX)

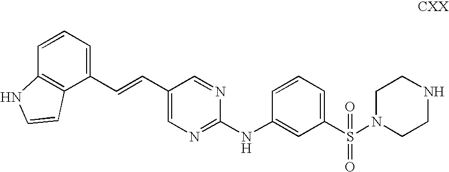

2-5 mL Emrys microwave vial was charged with the intermediate 3 (94.5 mg, 0.4 mmol), 4-[(3-bromophenyl)sulfonyl]piperazine-1-carboxylic acid tert-butyl ester (178.3 mg, 0.44 mmol), Pd(OAc)₂ (9.0 mg, 0.04 mmol), Xantphos (35 mg, 0.06 mmol), cesium carbonate (196.0 g, 0.6 mmol) and anhydrous dioxane (5 mL). The mixture was purged with argon gas for 10 min, then sealed and irradiated in a microwave (Initiator, Biotage) at 160° C. for 30 min. After cooling to room temperature, the cap was removed and the reaction mixture was poured into ca. 100 mL of DCM. The resulting solution was washed with dilute aqueous HCl (2×100 mL), brine (2×100 mL), dried over anhydrous Na₂SO₄, filtered and concentrated in vacuo. The residue was treated with 50% TFA in DCM for 1 h. 5 mL of CH₃CN were added, the reaction mixture was concentrated in vacuo to the total volume of 5 mL, filtered though 0.2 micron syringe filter and purified by reverse-phase preparative HPLC in CH₃CN/H₂O system containing 0.05% TFA. Fractions containing the product were combined and poured into EtOAc (50 mL). The solution was treated with saturated aqueous NaHCO₃ (2×30 mL), washed with brine (2×30 mL), dried over anhydrous Na₂SO₄ and filtered. The filtrate was concentrated in vacuo to give the title compound as a yellow solid (36.5 mg, 20%).

¹H NMR (500 MHz, DMSO-d₆): δ 2.74 (s, 3H), 2.81-2.85 (m, 4H), 2.91-2.95 (m, 4H), 6.91-6.93 (m, 1H), 7.10 (t, J=7.7 Hz, 1H), 7.23 (d, J=16.6 Hz, 1H), 7.25-7.27 (m, 1H), 7.29 (d, J=7.3 Hz, 1H), 7.33 (d, J=7.9 Hz, 1H), 7.42 (t, J=2.7 Hz, 1H), 7.55 (t, J=7.9 Hz, 1H), 7.66 (d, J=16.7 Hz, 1H), 8.03 (m, 1H), 8.31 (t, J=1.9 Hz, 2H), 8.92 (s, 2H), 10.16 (s, 1H), 11.19 (s, 1H). MS (ES+): m/z 461 (M+H)⁺.

### Example 273

### 6-[(E)-2-(2-{[4-(Piperidin-4-ylsulfonyl)phenyl]amino}pyrimidin-5-yl)vinyl]-1,3-benzothiazol-2-amine (Compound CXXI)

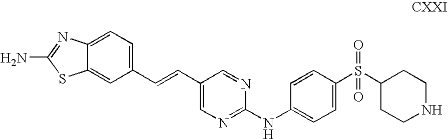

20 mL Emrys microwave vial was charged with the intermediate 6 (222.3 mg, 0.5 mmol), 2-amino-2-amino-2-amino-6-bromobenzothiazole (229.1 mg, 1.0 mmol), Pd₂(dba)₃ (45.8 mg, 0.05 mmol), tri(tert-butyl)phosphine (0.2 mL of 1.0 M solution in toluene, 0.2 mmol), cesium carbonate 325.8 g, 1.0 mmol) and anhydrous dioxane (20 mL). The reaction mixture was purged with argon gas for 20 min, then sealed and irradiated in a microwave (Initiator, Biotage) at 180° C. for 60 min. After cooling to ambient temperature, the reaction mixture was diluted with ca. 100 mL of EtOAc and filtered though a short pad of silica gel. The silica gel pad was washed with EtOAc. Combined organic solutions were concentrated in vacuo with ca. 15 g of silica gel. The loaded silica gel was taken to the ISCO system for further purification (40 g column, solid method, 0% to 100% EtOAc gradient in hexanes, 30 min method). Fractions containing the product were combined and concentrated in vacuo to give a yellow solid. The solid was re-crystallized from 15 mL of EtOAc to give the Boc-protected product as a light-yellow solid (158.0 mg, 53%). This solid was treated with 50% TFA in DCM for 10 min and the resulting solution was concentrated in vacuo to give yellow oil. The oil was re-dissolved in 15 mL of MeOH and this methanolic solution was added to ca. 200 ml of saturated aqueous NaHCO₃. The formed precipitated was collected, washed with water (3×40 mL), MeOH (1×20 mL), Et₂O (4×40 mL) and dried in vacuo to give the title compound as a bright-yellow solid (112 mg, 85%).

¹H NMR (500 MHz, DMSO-d₆): δ 1.32 (dq, J=12.3, 4.1, 2H), 1.74 (d, J=11.2 Hz, 2H), 2.38 (t, J=11.5 Hz, 2H), 2.95 (d, J=12.3 Hz, 2H), 3.15 (tt, J=12.1, 3.6 Hz, 1H), 7.07 (d, J=16.6 Hz, 1H), 7.32 (d, J=16.6 Hz, 1H), 7.33 (d, J=8.5 Hz, 1H), 7.44 (dd, J=8.5, 1.7 Hz, 1H), 7.57 (br s, 2H), 7.72 (d, J=9.0 Hz, 2H), 7.88 (d, J=1.5 Hz, 1H), 8.04 (d, J=9.0 Hz, 2H), 8.81 (s, 2H), 10.35 (s, 1H). MS (ES+): m/z 493 (M+H)⁺.

### Example 274

### N-Oxide 7-azaindole (Intermediate 151)

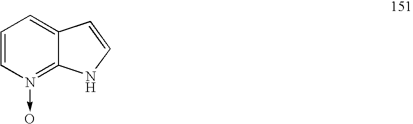

A solution of 7-azaindole (10 g, 84.6 mmol) in 100 mL of EtOAc was cooled down to 0+5° C. in a water-ice bath. A solution of meta-chloro-perbenzoic acid (19.0 g, 110.0 mmol) in 50 mL of EtOAc was added slowly dropwise to the solution of 7-azaindole via an addition funnel. After the completion of addition, the reaction mixture was allowed to warm up and stirred at ambient temperature for 5 h. Then it was cooled down again to 0+5° C. and kept at this temperature for 2 h. The formed precipitate was collected by filtration, washed with a minimum amount of cold EtOAc and dried in vacuo. The resulting meta-chlorobenzoic acid salt of the product was re-dissolved in 100 mL of DCM. The solution was washed with saturated aqueous NaHCO₃ (3×100 mL), brine (2×100 mL), dried over anhydrous Na₂SO₄, filtered and concentrated in vacuo to afford the title product (6.1 g, 54%).

¹H NMR (500 MHz, DMSO-d₆): δ 6.58 (d, J=3.2, 1H), 7.07 (dd, J=8.0, 6.3 Hz, 1H), 7.45 (d, J=3.3 Hz, 1H), 7.64 (d, J=7.8, 1H), 8.13 (d, J=6.1 Hz, 1H), 12.48 (s, 1H). MS (ES+): m/z 135 (M+H)⁺.

### Example 275

### 4-Bromo-1H-pyrrolo[2,3-b]pyridine (Intermediate 152)

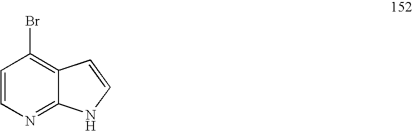

100 mL round-bottom flask was charged with intermediate 151 (1.86 g, 13.86 mmol) and 35 mL of anhydrous DMF. To the formed yellow solution solid tetramethylammonium bromide (4.28 g, 27.72 mmol) was added, followed by neat methanesulfonic anhydride (4.84 g, 27.72 mmol). The resulting suspension was heated to ca. 70° C. and stirred at 70° C. for 2 h. Then it was cooled down to ambient temperature and poured into ca 150 mL of H₂O. The formed clear brown solution was cooled down to 0+5° C. and 9 N NaOH solution was added to it dropwise until pH reached 7. The resulting solution was extracted with EtOAc (2×50 mL) and with DCM (2×50 mL). The combined organic extracts were washed with brine (3×100 mL), dried over anhydrous Na₂SO₄, filtered and concentrated in vacuo to give the crude product as a reddish-brown oil (2.7 g, 99%). This product was taken to the next step without further purification. MS (ES+): m/z 198 (M+H)⁺.

### Example 276

### 4-Bromo-1-(triisopropylsilyl)-1H-pyrrolo[2,3-b]pyridine (Intermediate 153)

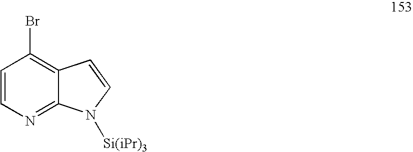

To a solution of intermediate 152 (2.7 g, 13.70 mmol) in 50 mL of anhydrous THF under argon atmosphere was added TIPSCl (2.93 g, 13.70 mmol, 90% purity) via a syringe. Then 95% dry NaH (0.38 g, 15.07 mmol) was added in small portions. The resulting mixture was left to stir at ambient temperature for 2 h. The completion of the reaction was monitored by silica gel TLC with 1:1 mixture of EtOAc/Hexanes. Upon the completion the reaction was quenched with aqueous NH₄Cl (200 mL) and this mixture was extracted with EtOAc (3×100 mL). The combined EtOAc extracts were washed with brine (3×100 mL), dried over anhydrous Na₂SO₄, filtered and concentrated in vacuo with ca. 15 g of silica gel. The loaded silica gel was taken to the ISCO system for purification (80 g column, solid method, 0 to 20% EtOAc gradient in 30 min). Fractions containing the product were combined and solvent was removed in vacuo to give the title intermediate as clear colorless oil (2.67 g, 55%).

¹H NMR (500 MHz, DMSO-d₆): δ 1.05 (d, J=7.5 Hz, 18H), 1.85 (sep, J=7.5 Hz, 3H), 6.59 (d, J=3.6 Hz, 1H), 7.36 (d, J=5.0 Hz, 1H), 7.59 (d, J=3.5 Hz, 1H), 8.09 (d, J=5.1 Hz, 1H).

### Example 277

### N-[4-(piperidin-4-ylsulfonyl)phenyl]-5-[(E)-2-(1H-pyrrolo[2,3-b]pyridin-4-yl)vinyl]pyrimidin-2-amine (Compound CXXII)

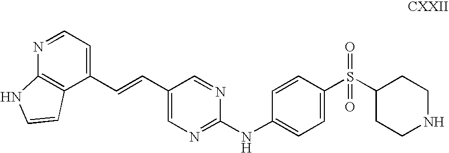

2-5 mL microwave vial was charged with intermediate 6 (88.91 mg, 0.2 mmol), intermediate 153 (141.4 mg, 0.4 mmol), Pd₂(dba)₃ (18.3 mg, 0.02 mmol), P(t-Bu)₃ (0.08 mL, 0.08 mmol, 1.0 M solution in toluene), cesium carbonate (130.3 g, 0.4 mmol) and anhydrous dioxane (5 mL). The mixture was purged with argon gas for 10 min, then sealed and irradiated in a microwave (Initiator, Biotage) at 200° C. for 60 min. After cooling to room temperature, the resulting mixture was diluted with ca. 50 mL of EtOAc, filtered though a short pad of silica gel and concentrated in vacuo to give a yellow solid. The solid was purified by silica gel chromatography using 1:1 mixture of EtOAc/hexanes as eluent. Fractions containing the product were combined and solvent was removed in vacuo to give Boc- and TIPS-protected product as yellow oil (60.4 mg, 42%). The oil was re-dissolved in 2 mL of anhydrous CH₃CN and neat TMSI (46 uL, 0.336 mmol) was added via a syringe. The reaction mixture was stirred at ambient temperature for 2 h. Then it was filtered though 0.2 micron syringe filter and purified by reverse-phase preparative HPLC in CH₃CN/H₂O system containing 0.05% of TFA. Fractions containing the product were combined and treated with saturated aqueous NaHCO₃ (30 mL). The resulting precipitate was collected by centrifugation, washed with water (2×40 mL), MeOH (1×10 mL), Et₂O (2×30 mL) and dried in vacuo to give the title compound as a light-yellow solid (2.7 mg, 7%).

¹H NMR (500 MHz, DMSO-d₆): δ 1.32 (dq, J=12.3, 4.2 Hz, 2H), 1.74 (d, J=10.8 Hz, 2H), 2.38 (t, J=11.2 Hz, 2H), 2.95 (d, J=12.3 Hz, 2H), 3.16 (tt, J=12.1, 3.5 Hz, 1H), 6.95 (d, J=3.5 Hz, 1H), 7.29 (d, J=5.1 Hz, 1H), 7.52 (d, J=14.2 Hz, 1H), 7.53 (d, J=3.5 Hz, 1H), 7.70 (d, J=16.6 Hz, 1H), 7.74 (d, J=9.0 Hz, 2H), 8.04 (d, J=9.0 Hz, 2H), 9.00 (s, 2H), 10.47 (br s, 1H), 11.71 (br s, 1H). MS (ES+): m/z 461 (M+H)⁺.

### Example 278

### tert-Butyl 4-(4-bromo-phenoxy)piperidine-1-carboxylate (Intermediate 154)

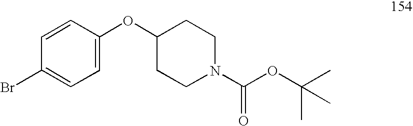

To a suspension of 4-(4-bromo-phenoxy)-piperidine hydrochloride salt (5.0 g, 17.08 mmol) in 100 mL of anhydrous THF was added neat Et3N (1.73 g, 17.08 mmol), followed by solid DMAP (626.2 mg, 5.12 mmol). After stirring for 15 min, solid di-tert-butyl dicarbonate (4.10 g, 18.8 mmol) was added and the reaction mixture was left to stir at ambient temperature overnight. The formed white suspension was concentrated down in vacuo to afford a white solid. The solid was re-suspended in ca. 50 mL of EtOAc and the suspension was passed though a short pad of silica gel. The silica gel pad was washed with ca. 200 mL of 1:1 mixture of EtOAc/Hexanes. The combined organic solution was concentrated in vacuo to give the title intermediate as yellow oil, which upon standing slowly solidified into a yellow solid (5.57 g, 92%). MS (ES+): m/z 357 (M+H)⁺.

### Example 279

### tert-Butyl 4-{4-[(5-vinylpyrimidin-2-yl)amino]phenoxy}piperidine-1-carboxylate (Intermediate 155)

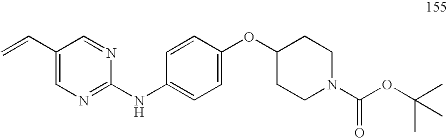

20 mL microwave vial was charged with the intermediate 1 (363.4 mg, 3.0 mmol), intermediate 154 (1.17 g, 3.3 mmol), Pd₂(dba)₃ (109.9 mg, 0.12 mmol), Xantphos (208.3 mL, 0.36 mmol), cesium carbonate (1.96 g, 6.0 mmol) and anhydrous dioxane (20 mL). The mixture was purged with argon gas for 10 min, then sealed and irradiated in a microwave (Initiator, Biotage) at 140° C. for 3 h. After cooling to room temperature, the resulting mixture was diluted with ca. 150 mL of EtOAc, filtered though a short pad of silica gel and concentrated in vacuo with ca. 10 g of silica gel. The loaded silica gel was taken to the ISCO system for purification (80 g column, solid method, 254 nm detection wavelength, 0 to 50% gradient of EtOAc in hexanes in 25 min, 40 min total). Fractions containing the product were combined and solvent was removed in vacuo to give the title intermediate as a white solid (241 mg, 20%).

¹H NMR (500 MHz, DMSO-d₆): δ 1.40 (s, 9H), 1.44-1.53 (m, 2H), 1.85-1.90 (m, 2H), 3.14-3.18 (m, 2H), 3.62-3.66 (m, 2H), 4.44 (sext, J=3.8 Hz, 1H), 5.18 (d, J=12.0 Hz, 1H), 5.81 (d, J=17.7 Hz, 1H), 6.59 (dd, J=17.9, 11.2 Hz, 1H), 6.91 (d, J=9.1 Hz, 2H), 7.62 (d, J=9.1 Hz, 2H), 8.57 (s, 2H), 9.57 (s, 1H). MS (ES+): m/z 397 (M+H)⁺.

### Example 280

### 5-[(E)-2-(1H-Indol-4-yl)vinyl]-N-[4-(piperidin-4-yloxy)phenyl]pyrimidin-2-amine (CXXXIII)

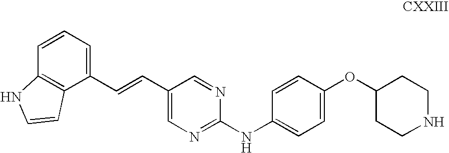

2-5 ml microwave vial was charged with the intermediate 154 (80.0 mg, 0.202 mmol), 4-bromo-indole-1-carboxylic acid tert-butyl ester (71.7 mg, 0.242 mmol), Pd₂(dba)₃ (18.5 mg, 0.020 mmol), P(t-Bu)₃ (0.08 mL, 0.08 mmol, 1.0 M solution in toluene), cesium carbonate (131.5 mg, 0.403 mmol) and anhydrous dioxane (5 mL). The mixture was purged with argon gas for 10 min, then sealed and irradiated in a microwave (Initiator, Biotage) at 200° C. for 30 min. After cooling to room temperature, the cap was removed and the resulting mixture was concentrated in vacuo. The residue was re-dissolved in 5 mL of DMF, filtered though 0.2 micron syringe filter and purified by reverse-phase preparative HPLC in CH₃CN/H₂O system containing 0.05% of TFA. All fractions, containing mono-Boc, di-Boc and de-Boc product, were combined, dried in vacuo, treated with 20% TFA in DCM for 30 min and subjected to a second purification by reverse-phase preparative HPLC in CH₃CN/H₂O system containing 0.05% of TFA. Fractions containing the product were combined and solvent was removed in vacuo to give the TFA salt of the title compound as a brownish solid (7.0 mg, 7%).

¹H NMR (500 MHz, DMSO-d₆): δ 1.78-1.83 (m, 2H), 2.03-2.1 (m, 2H), 3.04-3.12 (m, 2H), 3.21-3.28 (m, 2H), 4.56 (m, 1H), 6.91 (m, 1H), 7.10 (t, J=7.7 Hz, 1H), 7.20 (d, J=16.7 Hz, 1H), 7.29 (d, J=7.3 Hz, 1H), 7.33 (d, J=8.1 Hz, 1H), 7.42 (t, J=2.7 Hz, 1H), 7.58 (d, J=16.7 Hz, 1H), 7.68 (d, J=9.0 Hz, 2H), 8.50 (br s, 2H), 8.80 (s, 2H), 9.62 (s, 1H), 11.20 (s, 1H). MS (ES+): m/z 412 (M+H)⁺.

### Example 281

### 1-Benzoyl-4-bromo-1H-indazole (Intermediate 156)

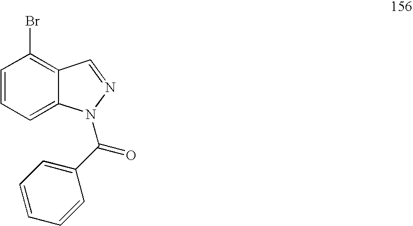

To a suspension of 4-bromo-1H-indazole (1.97 g, 10.0 mmol) in 60 mL of DCM was added Et₃N (1.11 g, 1.53 mL, 11.0 mmol) and the mixture was stirred at ambient temperature until all solids dissolved (ca. 15-20 min). Then neat benzoyl chloride (1.55 g, 1.27 mL, 11.0 mmol) was added via a syringe and the reaction mixture was left to stir at ambient temperature overnight. The completion of the reaction was monitored by silica gel TLC with 1:1 mixture of EtOAc/Hexanes. The reaction mixture was washed with water (2×100 mL), sat. NaHCO₃ (2×100 mL), brine (2×100 mL), dried over anhydrous Na₂SO₄, filtered and concentrated in vacuo to afford the title intermediate as a tan solid (3.0 g, 99%). MS (ES+): m/z 302 (M+H)⁺.

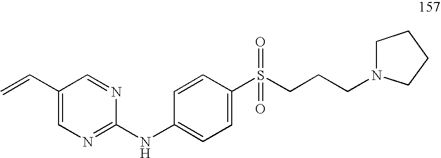

### Example 282

### N-{4-[(3-Pyrrolidin-1-ylpropyl)sulfonyl]phenyl}-5-vinylpyrimidin-2-amine (Intermediate 157)

100 mL round-bottom flask was charged with the intermediate 1 (605.7 mg, 5.0 mmol), 1-{3-[(4-bromophenyl)sulfonyl]propyl}pyrrolidine (1.66 g, 5.0 mmol), Pd₂(dba)₃ (183.1 mg, 0.2 mmol), Xantphos (347.2 mg, 0.6 mmol), cesium carbonate (3.25 g, 10.0 mmol) and 80 mL of anhydrous dioxane. The reaction mixture was purged with the argon gas for 30 min; then it was heated to reflux and refluxed under argon atmosphere for 18 h. The completion of the reaction was monitored by TLC and LC/MS. Upon completion the reaction mixture was cooled down to ambient temperature, diluted with ca. 100 mL of EtOAc and filtered though a short pad of silica gel. The silica gel pad was washed with EtOAc. Combined organic solutions were concentrated in vacuo with ca. 15 g of silica gel. The loaded silica gel was taken to the ISCO system for further purification (80 g column, solid method, 0% to 20% MeOH gradient in DCM with 0.2% of Et₃N, 40 min method, 310 nm detection wavelength). Fractions containing the product were combined and concentrated in vacuo to give the title intermediate as a tan solid (1.33 g, 72%).

¹H NMR (500 MHz, DMSO-d₆): δ 1.60-1.73 (m, 6H), 2.32-2.48 (m, 6H), 3.22-3.25 (m, 2H), 5.29 (d, J=11.7 Hz, 1H), 5.92 (d, J=17.7 Hz, 1H), 6.66 (dd, J=17.9, 11.2 Hz, 1H), 7.79 (d, J=8.9 Hz, 2H), 8.03 (d, J=8.9 Hz, 2H), 8.73 (s, 2H), 10.35 (s, 1H). MS (ES+): m/z 373 (M+H)⁺.

### Example 283

### 5-[(E)-2-(1H-Indazol-4-yl)vinyl]-N-{4-[(3-pyrrolidin-1-ylpropyl)sulfonyl]phenyl}pyrimidin-2-amine (Compound CXXIV)

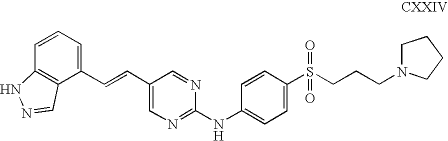

2-5 ml microwave vial was charged with intermediate 156 (165.6 mg, 0.55 mmol), intermediate 157 (186.2 mg, 0.5 mmol), Pd₂(dba)₃ (45.8 mg, 0.05 mmol), P(t-Bu)₃ (0.2 mL, 0.2 mmol, 1.0 M solution in toluene), cesium carbonate (325.8 mg, 1.0 mmol) and anhydrous dioxane (5 mL). The mixture was purged with argon gas for 10 min, then sealed and irradiated in a microwave (Initiator, Biotage) at 200° C. for 30 min. After cooling to room temperature, the cap was removed and the resulting mixture was concentrated in vacuo. The residue was re-dissolved in 5 mL of DMF, left to stir overnight, then filtered though 0.2 micron syringe filter and purified by reverse-phase preparative HPLC in CH₃CN/H₂O system containing 0.05% of TFA. All fractions containing the product were combined and poured into ca. 100 mL of EtOAc. EtOAc solution was washed with sat. NaHCO₃ (2×100 mL), brine (2×100 mL), dried over anhydrous Na₂SO₄, filtered and concentrated in vacuo to afford the title product as a light-yellow solid (71.5 mg, 29%).

¹H NMR (500 MHz, DMSO-d₆): δ 1.60-1.73 (m, 6H), 2.32-2.48 (m, 6H), 3.22-3.26 (m, 2H), 7.34-7.38 (m, 2H), 7.42 (d, J=16.7 Hz, 1H), 7.46-7.48 (m, 1H), 7.72 (d, J=16.7 Hz, 1H), 7.81 (d, J=8.9 Hz, 2H), 8.05 (d, J=8.9 Hz, 2H), 8.60 (s, 1H), 8.99 (s, 2H), 10.41 (s, 1H), 13.19 (s, 1H). MS (ES+): m/z 489.3 (M+H)⁺.

### Example 284

### 6-{(E)-2-[2-({4-[(3-Pyrrolidin-1-ylpropyl)sulfonyl]phenyl}amino)pyrimidin-5-yl]vinyl}-1,3-benzothiazol-2-amine (Compound CXXV)

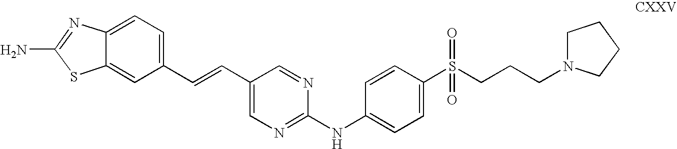

20 mL microwave vial was charged with intermediate 157 (372.5 mg, 1.0 mmol), 2-amino-2-amino-2-amino-6-bromobenzothiazole (275 mg, 1.3 mmol), P(t-Bu)₃ (0.4 mL, 0.4 mmol, 1.0 M solution in toluene), Pd₂(dba)₃ (91.6 mg, 0.1 mmol), cesium carbonate (651.6 g, 2.0 mmol) and anhydrous dioxane (15 mL). The mixture was purged with argon gas for 20 min, then sealed and irradiated in a microwave (Initiator, Biotage) at 180° C. for 40 min. After cooling to room temperature, the resulting mixture was diluted with ca. 150 mL of EtOAc and concentrated in vacuo with ca. 15 g of silica gel. The loaded silica gel was taken to the ISCO system for further purification (80 g column, solid method, 0% to 20% MeOH gradient in DCM with 0.2% of Et₃N, 45 min method, 310 nm detection wavelength). Fractions containing the product were combined and concentrated in vacuo to give the crude product with ca. 80% purity. The product was re-dissolved in 5 mL of DMF, filtered though 0.2 micron syringe filter and re-purified by reverse-phase preparative HPLC in CH₃CN/H₂O system containing 0.05% of TFA. All fractions containing the product were combined and poured into ca. 100 mL of EtOAc. EtOAc solution was washed with sat. NaHCO₃ (2×100 mL), brine (2×100 mL), dried over anhydrous Na₂SO₄, filtered and concentrated in vacuo to afford the title product as a light-yellow solid (131.0 mg, 25%).

¹H NMR (500 MHz, DMSO-d₆): δ 1.59-1.64 (m, 4H), 1.64-1.70 (m, 2H), 2.30-2.33 (m, 4H), 2.39 (t, J=7.0 Hz, 2H), 3.22-3.25 (m, 2H), 7.07 (d, J=16.6 Hz, 1H), 7.31 (d, J=8.5 Hz, 1H), 7.32 (d, J=16.8 Hz, 1H), 7.44 (dd, J=8.4, 1.7 Hz, 1H), 7.57 (s, 2H), 7.79 (d, J=8.9 Hz, 2H), 7.88 (d, J=1.6 Hz, 1H), 8.04 (d, J=8.9 Hz, 2H), 8.80 (s, 2H), 10.34 (s, 1H). MS (ES+): m/z 521 (M+H)⁺.

### Example 285

### 5-[(E)-2-(1H-indazol-4-yl)vinyl]-N-[4-(piperidin-4-yloxy)phenyl]pyrimidin-2-amine (Compound CXXVI)

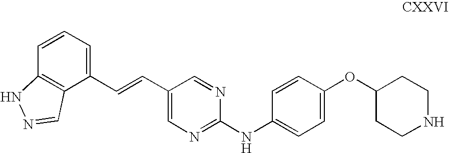

2-5 ml microwave vial was charged with intermediate 155 (198.2 mg, 0.5 mmol), intermediate 156 (165.6 mg, 0.55 mmol), Pd₂(dba)₃ (45.8 mg, 0.05 mmol), P(t-Bu)₃ (0.2 mL, 0.2 mmol, 1.0 M solution in toluene), cesium carbonate (325.8 mg, 1.0 mmol) and anhydrous dioxane (5 mL). The mixture was purged with argon gas for 20 min, then sealed and irradiated in a microwave (Initiator, Biotage) at 200° C. for 60 min. After cooling to room temperature, the cap was removed and the resulting mixture was concentrated in vacuo. The residue was re-dissolved in 5 mL of DMF, filtered though 0.2 micron syringe filter and purified by reverse-phase preparative HPLC in CH₃CN/H₂O system containing 0.05% of TFA. All fractions, containing mono-Boc product, were combined, dried in vacuo, treated with 50% TFA in DCM for 10 min, concentrated in vacuo and subjected to a second purification by reverse-phase preparative HPLC in CH₃CN/H₂O system containing 0.05% of TFA. Fractions containing the de-Boc product were combined and solvent was removed in vacuo to give the TFA salt of The title compound as a bright-yellow solid (54.2 mg, 21%).

¹H NMR (500 MHz, DMSO-d₆): δ 1.78-1.84 (m, 2H), 2.05-2.1 (m, 2H), 3.04-3.12 (m, 2H), 3.21-3.28 (m, 2H), 4.53-4.58 (m, 1H), 6.97 (d, J=9.1 Hz, 2H), 7.31-7.37 (m, 3H), 7.45 (d, J=7.8 Hz, 1H), 7.61 (d, J=16.7 Hz, 1H), 7.68 (d, J=9.1 Hz, 2H), 8.51 (br s, 1H), 8.56 (br s, 1H), 8.58 (s, 1H), 8.84 (s, 2H), 9.69 (s, 1H). MS (ES+): m/z 413.2 (M+H)⁺.

### Example 286

### 6-[(E)-2-(2-{[4-(piperidin-4-yloxy)phenyl]amino}pyrimidin-5-yl)vinyl]-1,3-benzothiazol-2-amine (Compound CXXVII)

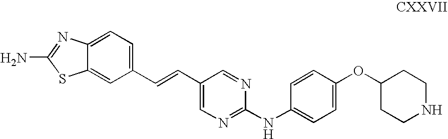

2-5 ml microwave vial was charged with intermediate 155 (198.2 mg, 0.5 mmol), 2-amino-6-bromobenzothiazole (229.1 mg, 1.0 mmol), Pd₂(dba)₃ (45.8 mg, 0.05 mmol), P(t-Bu)₃ (0.2 mL, 0.2 mmol, 1.0 M solution in toluene), cesium carbonate (325.8 mg, 1.0 mmol), Et₃N (2 drops) and anhydrous dioxane (5 mL). The mixture was purged with argon gas for 20 min, then sealed and irradiated in a microwave (Initiator, Biotage) at 200° C. for 60 min. After cooling to room temperature, the cap was removed and the resulting mixture was concentrated in vacuo. The residue was re-dissolved in 5 mL of DMF, filtered though 0.2 micron syringe filter and purified by reverse-phase preparative HPLC in CH₃CN/H₂O system containing 0.05% of TFA. All fractions, containing mono-Boc and de-Boc products, were combined, concentrated in vacuo and treated with 50% TFA in DCM for 10 min. The resulting solution was concentrated in vacuo and the residue was subjected to a second purification by reverse-phase preparative HPLC in CH₃CN/H₂O system containing 0.05% of TFA. Fractions containing the de-Boc product were combined and poured into ca. 100 mL of EtOAc. EtOAc solution was washed with saturated NaHCO₃ (2×100 mL), brine (2×100 mL), dried over anhydrous Na₂SO₄, filtered and concentrated in vacuo to afford the title product as a bright-yellow solid (85 mg, 38%).

¹H NMR (500 MHz, DMSO-d₆): δ 1.37-1.44 (m, 2H), 1.85-1.92 (m, 2H), 2.50-2.55 (m, 2H), 2.91-2.93 (m, 2H), 4.25-4.31 (m, 1H), 6.88 (d, J=9.0 Hz, 2H), 7.01 (d, J=16.6 Hz, 1H), 7.21 (d, J=16.5 Hz, 1H), 7.30 (d, J=8.3 Hz, 1H), 7.41 (dd, J=8.4, 1.4, 1H), 7.60-7.64 (m, 4H), 7.85 (d, J=1.2 Hz, 1H), 8.65 (s, 2H), 9.56 (s, 1H). MS (ES+): m/z 445.2 (M+H)⁺.

### Example 287

### tert-Butyl 4-{[4-({5-[(E)-2-(3,4-diaminophenyl)vinyl]pyrimidin-2-yl}amino)phenyl]sulfonyl}piperidine-1-carboxylate (Intermediate 158)

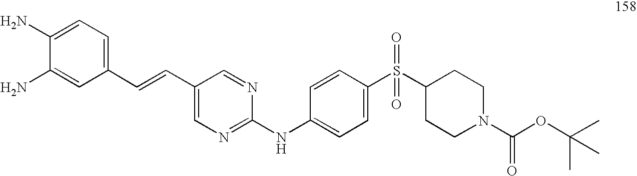

20 mL microwave vial was charged with intermediate 6 (444.5 mg, 1.0 mmol), 4-bromo-1,2-benzenediamine (374.0 mg, 2.0 mmol), P(t-Bu)₃ (0.8 mL, 0.8 mmol, 1.0 M solution in toluene), Pd₂(dba)₃ (91.6 mg, 0.1 mmol), cesium carbonate (651.6 g, 2.0 mmol), Et₃N (3 drops) and anhydrous dioxane (18 mL). The mixture was purged with argon gas for 20 min, then sealed and irradiated in a microwave (Initiator, Biotage) at 160° C. for 3 h. After cooling to room temperature, the resulting mixture was diluted with ca. 150 mL of EtOAc and filtered though a short pad of silica gel. The silica gel pad was washed with 20% MeOH in EtOAc. Combined organic solutions were concentrated in vacuo with ca. 15 g of silica gel. The loaded silica gel was taken to the ISCO system for further purification (80 g column, solid method, 0% to 10% MeOH gradient in EtOAc with 0.2% of Et₃N, 45 min method, 350 nm detection wavelength). Fractions containing the product were combined and concentrated in vacuo to afford the title intermediate as a reddish-yellow solid (216.5 mg, 39%).

¹H NMR (500 MHz, DMSO-d₆): δ 1.27-1.36 (m, 2H), 1.35 (s, 9H), 1.84 (d, J=11.8 Hz, 2H), 2.65-2.72 (m, 2H), 3.34 (tt, J=12.1, 3.6 Hz, 1H), 3.99 (m, 2H), 4.52 (br s, 2H), 4.74 (br s, 2H), 6.50 (d, J=8.0 Hz, 1H), 6.64 (dd, J=8.0, 1.8 Hz, 1H), 6.70 (d, J=16.5 Hz, 1H), 6.77 (d, J=1.7 Hz, 1H), 7.07 (d, J=16.5 Hz, 1H), 7.72 (d, J=8.9 Hz, 2H), 8.04 (d, J=9.0 Hz, 2H), 8.75 (s, 2H), 10.29 (s, 1H). MS (ES+): m/z 551.2 (M+H)⁺.

### Example 288

### 6-[(E)-2-(2-{[4-(Piperidin-4-ylsulfonyl)phenyl]amino}pyrimidin-5-yl)vinyl]-1H-benzimidazol-2-amine (Compound CXXVIII)

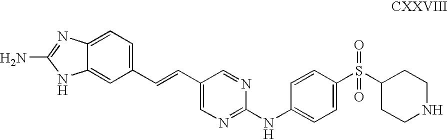

Intermediate 157 (216.5 mg, 0.39 mmol) was treated with 50% TFA in DCM (10 mL) for 30 min and the resulting solution was concentrated in vacuo. The residue was re-dissolved in 4 mL of DMF, filtered though 0.2 micron syringe filter and purified by reverse-phase preparative HPLC in CH₃CN/H₂O system containing 0.05% of TFA. All fractions containing the product were combined and treated with saturated aqueous NaHCO₃ (10 mL). The resulting precipitate was collected by centrifugation, washed with water (2×40 mL), MeOH (1×10 mL), Et₂O (2×30 mL) and dried in vacuo to give the title compound as a light-yellow solid (65.5 mg, 35%).

¹H NMR (500 MHz, DMSO-d₆): δ 1.33 (dq, J=12.3, 4.1 Hz, 2H), 1.74 (d, J=11.3 Hz, 2H), 2.39 (t, J=12.1 Hz, 2H), 2.96 (d, J=11.2 Hz, 2H), 3.13-3.17 (m, 1H), 6.24 (br s, 2H), 6.97 (d, J=16.6 Hz, 1H), 7.08 (d, J=8.1 Hz, 1H), 7.13 (d, J=8.1 Hz, 1H), 7.33 (d, J=16.5 Hz, 1H), 7.33 (s, 1H), 7.71 (d, J=8.9 Hz, 2H), 8.04 (d, J=8.9 Hz, 2H), 8.81 (s, 2H), 10.31 (s, 1H). MS (ES+): m/z 476 (M+H)⁺.

### Example 289

### N-(5-Bromopyridin-2-yl)-4-methylbenzenesulfonamide (Intermediate 159)

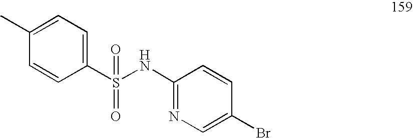

To a solution of 2-amino-5-bromo-pyridine (10.0 g, 57.8 mmol) in 50 mL of anhydrous pyridine was added p-toluenesulfonyl chloride (11.57 g, 60.7 mmol). The reaction mixture was heated to 70° C. and left to stir at 70° C. for 2 d. Then pyridine was removed in vacuo to give a light-yellow solid. The solid was crushed into a fine powder and suspended in ca. 200 mL of H₂O. The suspension was filtered, washed with water and dried in air with vacuum suction. The resulting solid was washed with ca. 200 mL of Et₂O and dried in vacuo to give the title intermediate as a fine white powder (17.3 g, 92%).

¹H NMR (500 MHz, DMSO-d₆): δ 7.04 (d, J=8.8 Hz, 1H), 7.36 (d, J=8.1 Hz, 2H), 7.79 (d, J=8.1 Hz, 2H), 7.88 (dd, J=8.8, 2.6 Hz, 1H), 8.27 (d, J=2.6 Hz, 1H), 11.21 (s, 1H). MS (ES+): m/z 327/329/330 (M+H)⁺.

### Example 290

### 2-[(2Z)-5-Bromo-2-{[(4-methylphenyl)sulfonyl]imino}pyridin-1-(2H)-yl]acetamide (Intermediate 160)

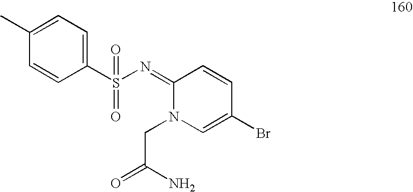

To a suspension of intermediate 159 (17.3 g, 52.87 mmol) in 50 mL of anhydrous DMF was added DIEA (6.83 g, 52.87 mmol), followed in 15 min by iodoacetamide (9.78 g, 52.87 mmol). The reaction mixture was left to stir at ambient temperature for 3 d. Then the reaction mixture was poured with vigorous stirring into ca. 1 L of H₂O. The aqueous solution was stirred for 1 h and then decanted. The resulting oily residue was heated to reflux in ca. 200 mL of MeOH until all the material dissolved. The resulting methanolic solution was allowed to cool down to ambient temperature over 1 h. The formed white solid was filtered, washed with ca. 50 mL of cold MeOH and dried in vacuo to give the title intermediate as a white fluffy solid (15.46 g, 76%).

¹H NMR (500 MHz, DMSO-d₆): δ 2.33 (s, 3H), 4.78 (s, 2H), 7.27 (d, J=8.1 Hz, 2H), 7.30 (d, J=9.7 Hz, 1H), 7.39 (br s, 1H), 7.65 (d, J=8.1 Hz, 2H), 7.78 (br s, 2H), 7.88 (dd, J=9.7, 2.6 Hz, 1H), 8.37 (d, J=2.6 Hz, 1H). MS (ES+): m/z 384/386 (M+H)⁺.

### Example 291

### N-(6-Bromoimidazo[1,2-a]pyridine-2-yl)-2,2,2-trifluoroacetamide (Intermediate 161)

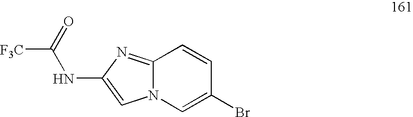

To a suspension of intermediate 160 (9.0 g, 23.42 mmol) in 150 mL of DCM was added trifluoroacetic acid anhydride (6.04 g, 28.75 mmol). The reaction mixture was left to stir at ambient temperature for 18 h. Then solvent was removed in vacuo to give an orange solid. The solid was partitioned in 200 mL of EtOAc and 200 mL of saturated aq. NaHCO₃. The layers were separated and the organic layer was washed with sat. NaHCO₃ (3×100 mL), brine (2×200 mL), dried over anhydrous Na₂SO₄, filtered and concentrated in vacuo. The residue was purified by silica gel chromatography using 50 to 100% gradient of EtOAc in hexanes. Fractions containing the product were combined and solvent was removed in vacuo to give the title intermediate as a yellow solid (4.2 g, 58%).

¹H NMR (500 MHz, DMSO-d₆): δ 7.40 (dd, J=9.5, 1.2 Hz, 1H), 7.50 (d, J=9.5 Hz, 1H), 8.23 (s, 1H), 8.95 (d, J=1.2 Hz, 1H), 12.52 (s, 1H). MS (ES+): m/z 309 (M+H)⁺.

### Example 292

### 2,2,2-Trifluoro-N-{6-[(E)-2-(2-{[4-(piperidin-4-ylsulfonyl)phenyl]amino}pyrimidin-5-yl)vinyl]imidazolo[1,2-a]pyridin-2-yl}acetamide (Compound CXXIX)

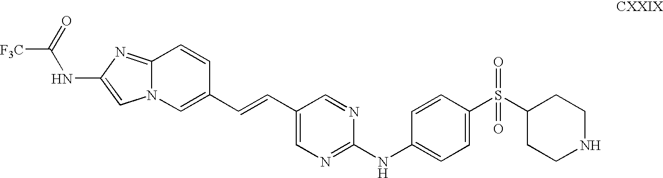

10 ml vial was charged with intermediate 6 (111.13 mg, 0.25 mmol), intermediate 161 (154.0 mg, 0.5 mmol), Pd(OAc)₂ (11.2 mg, 0.05 mmol), PPh₃ (26.2 mg, 0.1 mmol), sodium bicarbonate (42.0 mg, 0.5 mmol) and anhydrous DMF (5 mL). The mixture was purged with argon gas for 10 min and heated to 160° C. for 30 min. Then it was left to stir at 130° C. for 15 h. After cooling to room temperature, the reaction mixture was filtered though 0.2 micron syringe filter and purified by reverse-phase preparative HPLC in CH₃CN/H₂O system containing 0.05% of TFA. All fractions containing the product were combined and concentrated in vacuo to afford a light-yellow solid. The solid was treated with 50% TFA in DCM (10 mL) for 10 min. The resulting solution was concentrated in vacuo and the residue was subjected to a second purification by reverse-phase preparative HPLC in CH₃CN/H₂O system containing 0.05% of TFA. Fractions containing the product were combined and solvent was removed in vacuo to give the TFA salt of The title compound as a light-yellow solid (31.2 mg, 22%).

¹H NMR (500 MHz, DMSO-d₆): δ 1.67 (dq, J=13.0, 3.7 Hz, 2H), 2.03 (d, J=12.7 Hz, 2H), 2.88 (q, J=12.2 Hz, 2H), 3.35 (d, J=12.2 Hz, 2H), 3.48 (tt, J=12.2, 3.5 Hz, 1H), 7.21 (d, J=16.5 Hz, 1H), 7.33 (d, J=16.5 Hz, 1H), 7.57 (d, J=9.4 Hz, 1H), 7.71 (dd, J=9.6, 1.5 Hz, 1H), 7.76 (d, J=8.9 Hz, 2H), 8.09 (d, J=8.9 Hz, 2H), 8.24 (br s, 1H), 8.27 (s, 1H), 8.66 (br s, 1H), 8.68 (s, 1H), 10.47 (s, 1H), 12.49 (br s, 1H). MS (ES+): m/z 572 (M+H)⁺.

### Example 293

### 6-[(E)-2-(2-{[4-(Piperidin-4-ylsulfonyl)phenyl]amino}pyrimidin-5-yl)vinyl]imidazolo[1,2-a]pyridin-2-amine (Compound CXXX)

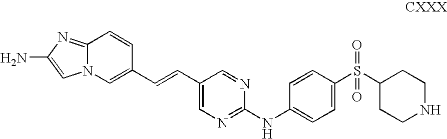

The above-described compound CXXIX (23 mg, 0.040 mmol) was dissolved in 2 mL of MeOH and 2 mL of saturated aqueous Na₂CO₃ solution was added. The reaction mixture was left to stir at ambient temperature overnight. Then the mixture was filtered though 0.2 micron syringe filter and purified by reverse-phase preparative HPLC in CH₃CN/H₂O system containing 0.05% of TFA. All fractions containing the product were combined and concentrated in vacuo to give the TFA salt of the title compound as a yellow solid (14.3 mg, 51%).

¹H NMR (500 MHz, DMSO-d₆): δ 1.64-1.71 (m, 2H), 2.03 (d, J=12.2 Hz, 2H), 2.86-2.90 (m, 2H), 3.35 (d, J=11.9 Hz, 2H), 3.48 (tt, J=12.0, 3.4 Hz, 1H), 7.28 (s, 1H), 7.29 (d, J=16.5 Hz, 1H), 7.36 (d, J=16.5 Hz, 1H), 7.67 (d, J=9.3 Hz, 1H), 7.77 (d, J=8.9 Hz, 2H), 7.94 (d, J=9.3 Hz, 1H), 8.09 (d, J=8.9 Hz, 2H), 8.27 (br s, 1H), 8.71 (br s, 1H), 8.75 (s, 1H), 8.87 (s, 2H), 10.52 (s, 1H). MS (ES+): m/z 476 (M+H)⁺.

### Example 294

### tert-Butyl 4-{[4-({5-[(E)-2-(4-cyano-3-trifluorophenyl)vinyl]pyrimidin-2-yl}amino)phenyl]sulfonyl}piperidine-1-carboxylate (Intermediate 162)

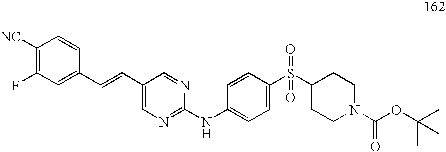

10 ml vial was charged with the intermediate 6 (444.6 mg, 1.0 mmol), 4-bromo-2-fluorobenzonitrile (240.0 mg, 1.2 mmol), Pd(OAc)₂ (45.0 mg, 0.2 mmol), PPh₃ (105.0 mg, 0.4 mmol), sodium bicarbonate (168.0 mg, 2.0 mmol) and anhydrous DMF (6 mL). The mixture was purged with argon gas for 10 min and heated at 150° C. for 1 h. After cooling to room temperature, the reaction mixture was poured into ca. 100 mL of H₂O. The resulting suspension was stirred at ambient temperature for 1 h, and then centrifuged down. The formed precipitate was suspended in 50 mL of MeOH. This methanolic solution was heated to reflux for 5 min and then cooled down to r.t. The precipitate was collected by centrifugation, washed with MeOH (2×40 mL), Et₂O (3×40 mL) and dried in vacuo to give the title intermediate as a grayish solid (402.5 mg, 71%). MS (ES+): m/z 564.1 (M+H)⁺.

### Example 295

### 6-[(E)-2-(2-{[4-(Piperidin-4-ylsulfonyl)phenyl]amino}pyrimidin-5-yl)vinyl]-1,2-benzisoxazol-3-amine (Compound CXXXI)

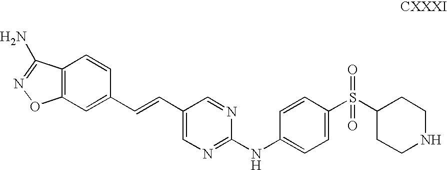

To a solution of acetohydroxamic acid (48.6 mg, 0.647 mmol) in 2 mL of anhydrous DMF was added solid potassium tert-butoxide (155.2 mg, 0.777 mmol). The formed suspension was stirred at ambient temperature for 15 min. To this suspension was added a solution of intermediate 162 (365.0 mg, 0.647 mmol) in 6 mL of DMF. The reaction mixture was stirred at ambient temperature for 24 h. Then the reaction mixture was poured into 100 mL of H₂O and the resulting solution was extracted with EtOAc (4×50 mL). The combined EtOAc extracts were washed with water (3×100 mL), brine (2×100 mL), dried over anhydrous Na₂SO₄, filtered though a short pad of silica gel and concentrated in vacuo to give the Boc-protected product as an orange solid. The solid was treated with 50% TFA in DCM (20 mL) for 10 min and the resulting solution was concentrated in vacuo. The residue was re-dissolved in 6 mL of DMF, filtered though 0.2 micron syringe filter and purified by reverse-phase preparative HPLC in CH₃CN/H₂O system containing 0.05% of TFA. All fractions containing the product were combined and treated with saturated aqueous NaHCO₃ (100 mL). The resulting precipitate was collected by centrifugation, washed with water (3×40 mL), MeOH (1×10 mL), Et₂O (3×40 mL) and dried in vacuo to give the title compound as an off-white solid (120.0 mg, 38%).

¹H NMR (500 MHz, DMSO-d₆): δ 1.33 (dq, J=12.3, 4.2 Hz, 2H), 1.74 (d, J=11.0 Hz, 2H), 2.36-2.40 (m, 2H), 2.94-2.98 (m, 2H), 3.14-3.18 (m, 1H), 6.39 (s, 2H), 7.35 (d, J=16.6 Hz, 1H), 7.46 (d, J=16.6 Hz, 1H), 7.50 (dd, J=8.4, 1.0 Hz, 1H), 7.60 (s, 1H), 7.73 (d, J=8.8 Hz, 2H), 7.81 (d, J=8.1 Hz, 1H), 8.05 (d, J=8.9 Hz, 2H), 8.86 (s, 2H), 10.43 (s, 1H). MS (ES+): m/z 477 (M+H)⁺.

### Example 296

### tert-Butyl 4-{[4-({5-[(E)-2-(3-cyano-2-trifluorophenyl)vinyl]pyrimidin-2-yl}amino)phenyl]sulfonyl}piperidine-1-carboxylate (Intermediate 163)

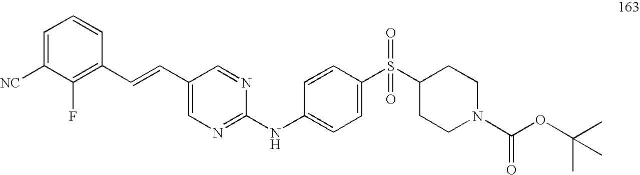

5 ml microwave vial was charged with the intermediate 6 (222.3 mg, 0.5 mmol), 3-bromo-2-fluorobenzonitrile (240.0 mg, 1.2 mmol), Pd(OAc)₂ (22.4 mg, 0.1 mmol), PPh₃ (52.5 mg, 0.2 mmol), sodium bicarbonate (84.0 mg, 1.0 mmol) and anhydrous DMF (5 mL). The mixture was purged with argon gas for 10 min, then sealed and irradiated in a microwave (Initiator, Biotage) at 160° C. for 30 min. After cooling to room temperature, the resulting mixture was filtered though 0.2 micron syringe filter and purified by reverse-phase preparative HPLC in CH₃CN/H₂O system containing 0.05% of TFA. All fractions containing the product were combined and poured into ca. 80 mL of EtOAc. EtOAc solution was washed with sat. NaHCO₃ (2×100 mL), brine (2×100 mL), dried over anhydrous Na₂SO₄, filtered and concentrated in vacuo to afford the title intermediate as a red solid (154.0 mg, 55%). MS (ES+): m/z 564.1 (M+H)⁺.

### Example 297

### 7-[(E)-2-(2-{[4-(Piperidin-4-ylsulfonyl)phenyl]amino}pyrimidin-5-yl)vinyl]-1,2-benzisoxazol-3-amine (CXXXII)

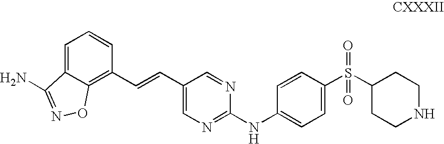

To a solution of acetohydroxamic acid (29.0 mg, 0.385 mmol) in 2 mL of anhydrous DMF was added solid potassium tert-butoxide (43.2 mg, 0.385 mmol). The formed suspension was stirred at ambient temperature for 15 min. To this suspension was added a solution of intermediate 163 (145.0 mg, 0.257 mmol) in 3 mL of DMF. The reaction mixture was stirred at ambient temperature for 24 h. Then the reaction mixture was poured into 100 mL of H₂O and the resulting solution was extracted with EtOAc (4×50 mL). The combined EtOAc extracts were washed with water (3×100 mL), brine (2×100 mL), dried over anhydrous Na₂SO₄, filtered though a short pad of silica gel and concentrated in vacuo to give the Boc-protected product as yellow oil. The oil was treated with 50% TFA in DCM (20 mL) for 10 min and the resulting solution was concentrated in vacuo. The residue was re-dissolved in 3 mL of DMF, filtered though 0.2 micron syringe filter and purified by reverse-phase preparative HPLC in CH₃CN/H₂O system containing 0.05% of TFA. All fractions containing the product were combined and treated with saturated aqueous NaHCO₃ (100 mL). The resulting precipitate was collected by centrifugation, washed with water (3×40 mL), MeOH (1×10 mL), Et₂O (3×40 mL) and dried in vacuo to give the title compound as a beige solid (52.5 mg, 43%).

¹H NMR (500 MHz, DMSO-d₆): δ 1.32 (dq, J=12.2, 4.1 Hz, 2H), 1.73 (d, J=11.0 Hz, 2H), 2.38 (d, J=11.4 Hz, 2H), 2.95 (d, J=12.3 Hz, 2H), 3.16 (tt, J=12.1, 3.5 Hz, 1H), 6.50 (s, 2H), 7.29 (t, J=7.6 Hz, 1H), 7.48 (d, J=16.7 Hz, 1H), 7.52 (d, J=16.7 Hz, 1H), 7.63 (d, J=7.0 Hz, 1H), 7.73 (d, J=8.9 Hz, 2H), 7.76 (d, J=0.8 Hz, 1H), 8.05 (d, J=8.9 Hz, 2H), 8.92 (s, 2H), 10.43 (s, 1H). MS (ES+): m/z 477 (M+H)⁺.

### Example 298

### 2-{[5-Bromo-2-(piperazin-1-ylsulfonyl)phenyl]amino}ethanol (Intermediate 164)

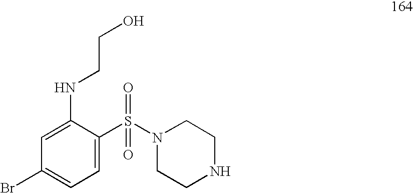

20 mL microwave vial was charged with 10 mL of ethanolamine and heated to ca. 60° C. Intermediate 128 (3.6 g, 8.5 mmol) was added in small portions with stirring and the mixture was stirred at 60° C. until clear. Then the vial was sealed and irradiated in a microwave (Initiator, Biotage) at 160° C. for 20 min. After cooling to room temperature, the reaction mixture was poured into ca. 200 mL of H₂O and this mixture was left to stir at ambient temperature overnight. The resulting precipitate was filtered, washed with H₂O (2×200 mL), Et₂O (1×100 mL) and dried in vacuo to give the title intermediate as a white solid (2.42 g, 78%).

### Example 299

### tert-Butyl 4-{[4-bromo-2-({2-[(tert-butoxycarbonyl)oxy]ethyl}amino)phenyl]sulfonyl}piperazine-1-carboxylate (Intermediate 165)

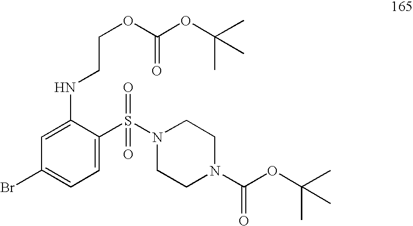

To a solution of intermediate 164 (3.05 g, 8.37 mmol) in 60 mL of anhydrous THF was added DMAP (102.2 mg, 0.837 mmol), followed by di-tert-butyl dicarbonate (4.02 g, 18.42 mmol). The reaction mixture was left to stir overnight. Then it was diluted with ca. 100 mL of EtOAc and filtered though a short pad of silica gel. Solvent was removed in vacuo to give the title intermediate as clear colorless oil, which slowly solidified into an off-white solid (4.7 g, 99%).

### Example 300

### tert-Butyl 4-({2-({2-[(tert-butoxycarbonyl)oxy]ethyl}amino)-4-[(5-vinylpyrimidin-2-yl)amino]phenyl}sulfonyl)piperazine-1-carboxylate (Intermediate 166)

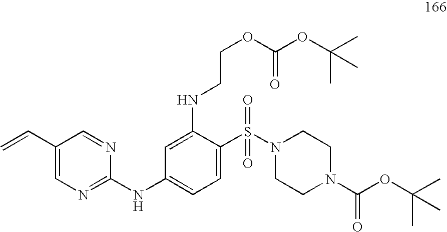

100 mL round-bottom flask was charged with intermediate 165 (2.258 g, 4.0 mmol), intermediate 1 (484.5 mg, 4.0 mmol), Pd₂(dba)₃ (146.5 mg, 0.16 mmol), Xantphos (277.7 mg, 0.48 mmol), cesium carbonate (2.60 g, 8.0 mmol) and anhydrous dioxane (70 mL). The mixture was purged with argon gas for 30 min, then brought to reflux and refluxed for 5 h under argon atmosphere. After cooling to room temperature, the resulting mixture was diluted with ca. 150 mL of EtOAc and filtered though a short pad of silica gel. The silica gel pad was washed with EtOAc. Combined organic solutions were concentrated in vacuo with ca. 15 g of silica gel. The loaded silica gel was taken to the ISCO system for further purification (80 g column, solid method, 0% to 50% EtOAc gradient in hexanes, 45 min method, 254 nm detection wavelength). Fractions containing the intermediate were combined and concentrated in vacuo to afford the title intermediate as a yellow solid (1.95 g, 81%).

¹H NMR (500 MHz, DMSO-d₆): δ 1.33 (s, 9H), 1.39 (s, 9H), 1.73 (d, J=11.0 Hz, 2H), 2.91 (t, J=4.8 Hz, 4H), 3.36 (t, J=4.8 Hz, 4H), 3.43 (q, J=5.3 Hz, 2H), 4.26 (t, J=5.2 Hz, 2H), 5.27 (d, J=11.5 Hz, 1H), 5.90 (d, J=18.2 Hz, 1H), 6.28 (t, J=5.4 Hz, 1H), 6.63 (dd, J=17.8, 11.3 Hz, 1H), 7.20 (dd, J=8.9, 1.9 Hz, 1H), 7.40 (d, J=8.9 Hz, 1H), 7.47 (d, J=1.8 Hz, 1H), 8.69 (s, 2H), 10.04 (s, 1H). MS (ES+): m/z 405/505/605 (M+H)⁺.

### Example 301

### 2-{[5-([5-[(E)-2-(2-Amino-1,3-benzothiazol-6-yl)vinyl]pyrimidin-2-yl]amino)-2-(piperazin-1-ylsulfonyl)phenyl]amino}ethanol (Compound CXXXIII)

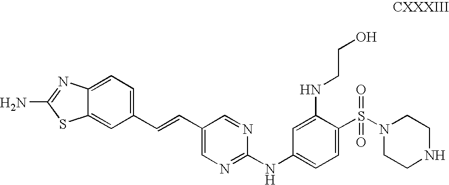

2-5 ml microwave vial was charged with intermediate 166 (302.3 mg, 0.5 mmol), 2-amino-6-bromobenzothiazole (137.4 mg, 0.6 mmol), Pd₂(dba)₃ (45.8 mg, 0.05 mmol), P(t-Bu)₃ (0.4 mL, 0.4 mmol, 1.0 M solution in toluene), cesium carbonate (325.8 mg, 1.0 mmol), Et₃N (2 drops) and anhydrous dioxane (5 mL). The mixture was purged with argon gas for 10 min, then sealed and irradiated in a microwave (Initiator, Biotage) at 180° C. for 40 min. After cooling to room temperature, the cap was removed and the resulting mixture was concentrated in vacuo. The residue was re-dissolved in 5 mL of DMF, filtered though 0.2 micron syringe filter and purified by reverse-phase preparative HPLC in CH₃CN/H₂O system containing 0.05% of TFA. All fractions, containing mono-Boc and di-Boc products, were combined, concentrated in vacuo and treated with 50% TFA in DCM for 10 min. The resulting solution was concentrated in vacuo and the residue was subjected to a second purification by reverse-phase preparative HPLC in CH₃CN/H₂O system containing 0.05% of TFA. Fractions containing the product were combined and poured into ca. 100 mL of saturated aqueous NaHCO₃. The resulting precipitate was collected by centrifugation, washed with water (2×40 mL), MeOH (1×5 mL), Et₂O (2×40 mL) and dried in vacuo to give the title compound as a light-yellow solid (51.0 mg, 18%).

¹H NMR (500 MHz, DMSO-d₆): δ 2.15 (br s, 1H), 2.67-2.69 (m, 2H), 2.82-2.86 (m, 2H), 3.18 (q, J=5.2 Hz, 2H), 3.64-3.68 (m, 2H), 4.88 (br s, 1H), 6.43 (t, J=4.8 Hz, 1H), 7.05 (d, J=16.6 Hz, 1H), 7.17 (dd, J=8.9, 1.7 Hz, 1H), 7.31 (d, J=16.6 Hz, 1H), 7.33 (d, J=8.3 Hz, 1H), 7.36 (d, J=8.9 Hz, 1H), 7.42-7.44 (m, 2H), 7.57 (br s, 2H), 7.87 (s, 1H), 8.77 (s, 2H), 10.00 (s, 1H). MS (ES+): m/z 553.2 (M+H)⁺.

### Example 302

### 2-{[5-({5-[(E)-2-(1H-Indazol-4-yl)vinyl]pyrimidin-2-yl}amino)-2-(piperazin-1-ylsulfonyl)phenyl]amino}ethanol (Compound CXXXIV)

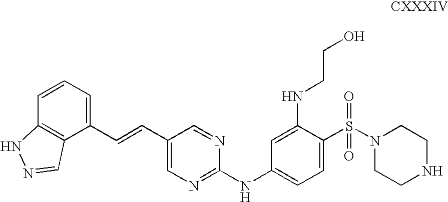

2-5 microwave vial was charged with intermediate 166 (302.3 mg, 0.5 mmol), 4-bromoindazole (118.2 mg, 0.6 mmol), Pd(OAc)₂ (22.5 mg, 0.01 mmol), PPh₃ (52.4 mg, 0.2 mmol), Et₃N (506.0 mg, 0.7 mL, 5.0 mmol) and anhydrous DMF (5 mL). The mixture was purged with argon gas for 10 min, then sealed and placed in a heating block at 150° C. for 3 h. After cooling to room temperature, the cap was removed and the resulting mixture was filtered though 0.2 micron syringe filter and purified by reverse-phase preparative HPLC in CH₃CN/H₂O system containing 0.05% of TFA. All fractions, containing mono-Boc and di-Boc products, were combined, concentrated in vacuo and treated with 50% TFA in DCM for 10 min. The resulting solution was concentrated in vacuo and the residue was subjected to a second purification by reverse-phase preparative HPLC in CH₃CN/H₂O system containing 0.05% of TFA. Fractions containing the product were combined and poured into ca. 100 mL of saturated aqueous NaHCO₃. The resulting precipitate was collected by centrifugation, washed with water (2×40 mL), MeOH (1×5 mL), Et₂O (2×40 mL) and dried in vacuo to give the title compound as a beige solid (126.1 mg, 48%).

¹H NMR (500 MHz, DMSO-d₆): δ 2.17 (br s, 1H), 2.69 (t, J=4.7 Hz, 2H), 2.85 (t, J=4.7 Hz, 2H), 3.19 (q, J=5.3 Hz, 2H), 3.67 (q, J=5.2 Hz, 2H), 4.89 (t, J=4.9 Hz, 1H), 6.45 (t, J=4.9 Hz, 1H), 7.18 (dd, J=8.9, 1.9 Hz, 1H), 7.34-7.39 (m, 3H), 7.40 (d, J=16.5 Hz, 1H), 7.45-7.47 (m, 2H), 7.70 (d, J=16.7 Hz, 1H), 8.60 (s, 1H), 8.95 (s, 2H), 10.07 (s, 1H), 13.81 (s, 1H). MS (ES+): m/z 521.2 (M+H)⁺.

### Example 303

### tert-Butyl 4-{[2-({[2-[(tert-butoxycarbonyl)oxy]ethyl}amino)-4-({5-[(E)-2-(3-cyano-2-fluorophenyl)vinyl]pyrimidin-2-yl}amino)phenyl]sulfonyl}piperazine-1-carboxylate (Intermediate 167)

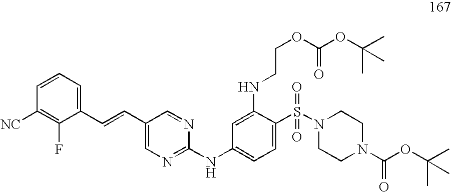

5 ml microwave vial was charged with intermediate 166 (282.2 mg, 0.5 mmol), 3-bromo-2-fluorobenzonitrile (120.0 mg, 0.6 mmol), Pd(OAc)₂ (22.4 mg, 0.1 mmol), PPh₃ (52.5 mg, 0.2 mmol), sodium bicarbonate (84.0 mg, 1.0 mmol) and anhydrous DMF (5 mL). The mixture was purged with argon gas for 10 min, then sealed and irradiated in a microwave (Initiator, Biotage) at 160° C. for 30 min. After cooling to room temperature, the resulting mixture was filtered though 0.2 micron syringe filter and purified by reverse-phase preparative HPLC in CH₃CN/H₂O system containing 0.05% of TFA. Fractions containing the product were combined and concentrated in vacuo to afford the title intermediate as a red solid (174.0 mg, 48%).

### Example 304

### 2-{[5-({5-[(E)-2-(3-Amino-1,2-benzisoxazol-7-yl)vinyl]pyrimidin-2-yl}amino)-2-(piperazin-1-ylsulfonyl)phenyl]amino}ethanol (Compound CXXXV)

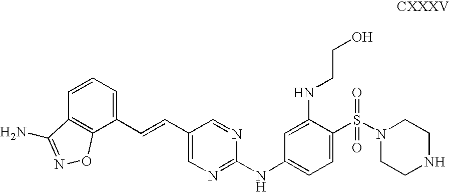

To a solution of acetohydroxamic acid (36.07 mg, 0.48 mmol) in 2 mL of anhydrous DMF was added solid potassium tert-butoxide (51.8 mg, 0.48 mmol). The formed suspension was stirred at ambient temperature for 15 min. To this suspension was added a solution of intermediate 167 (174.0 mg, 0.24 mmol) in 2 mL of DMF. The reaction mixture was stirred at ambient temperature for 24 h. Then the reaction mixture was poured into 100 mL of H₂O and the resulting solution was extracted with EtOAc (4×50 mL). The combined EtOAc extracts were washed with water (3×100 mL), brine (2×100 mL), dried over anhydrous Na₂SO₄, filtered and concentrated in vacuo to give the di-Boc-protected product as yellow solid. The solid was treated with 50% TFA in DCM (20 mL) for 10 min and the resulting solution was concentrated in vacuo. The residue was re-dissolved in 3 mL of DMF, filtered though 0.2 micron syringe filter and purified by reverse-phase preparative HPLC in CH₃CN/H₂O system containing 0.05% of TFA. All fractions containing the product were combined and treated with saturated aqueous NaHCO₃ (100 mL). The resulting precipitate was collected by centrifugation, washed with water (3×40 mL), MeOH (1×10 mL), Et₂O (3×40 mL) and dried in vacuo to give the title compound as a beige solid (51.0 mg, 40%).

¹H NMR (500 MHz, DMSO-d₆): δ 2.17 (br s, 1H), 2.69 (t, J=4.7 Hz, 2H), 2.83-2.87 (m, 2H), 3.18 (q, J=5.3 Hz, 2H), 3.66 (q, J=5.2 Hz, 2H), 4.87 (t, J=4.9 Hz, 1H), 6.44 (t, J=4.8 Hz, 1H), 6.48 (s, 2H), 7.18 (dd, J=8.8, 1.7 Hz, 1H), 7.29 (t, J=7.5 Hz, 1H), 7.38 (d, J=8.8 Hz, 1H), 7.42 (d, J=1.7 Hz, 1H), 7.46 (d, J=16.8 Hz, 1H), 7.50 (d, J=16.8 Hz, 1H), 7.62 (d, J=7.0 Hz, 1H), 7.74 (dd, J=7.8, 0.6 Hz, 1H), 8.89 (s, 2H), 10.07 (s, 1H). MS (ES+): m/z 537.2 (M+H)⁺.

### Example 305

### 3-{(E)-2-[2-({3-[(2-Hydroxyethyl)amino]-4-(piperazin-1-ylsulfonyl)phenyl}amino)pyrimidin-5-yl]vinyl}phenol (CXXXVI)

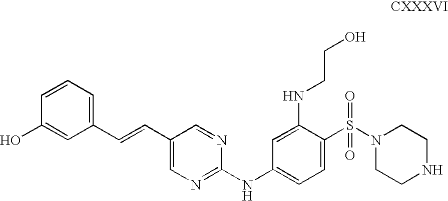

5 ml microwave vial was charged with intermediate 166 (282.2 mg, 0.5 mmol), 3-bromophenol (103.8 mg, 0.6 mmol), Pd(OAc)₂ (22.4 mg, 0.1 mmol), PPh₃ (52.5 mg, 0.2 mmol), Et₃N (506.0 mg, 0.7 mL, 5.0 mmol) and anhydrous DMF (5 mL). The mixture was purged with argon gas for 10 min, then sealed and irradiated in a microwave (Initiator, Biotage) at 160° C. for 70 min. After cooling to room temperature, the resulting mixture was filtered though 0.2 micron syringe filter and purified by reverse-phase preparative HPLC in CH₃CN/H₂O system containing 0.05% of TFA. Fractions containing the di-Boc protected product were combined and concentrated in vacuo to afford the di-Boc-protected product as yellow solid. The solid was treated with 50% TFA in DCM (20 mL) for 10 min and the resulting solution was concentrated in vacuo. The residue was re-dissolved in 3 mL of DMF, filtered though 0.2 micron syringe filter and purified by reverse-phase preparative HPLC in CH₃CN/H₂O system containing 0.05% of TFA. All fractions containing the product were combined and solvent was removed in vacuo to give the TFA salt of The title product as a yellow solid (26.5 mg, 9%).

¹H NMR (500 MHz, DMSO-d₆): δ 3.12-3.21 (m, 10H), 3.65-3.69 (m, 2H), 6.40 (br s, 1H), 6.70 (d, J=7.5 Hz, 1H), 6.95 (s, 1H), 7.00 (d, J=7.2 Hz, 1H), 7.06 (d, J=16.9 Hz, 1H), 7.16-7.20 (m, 2H), 7.25 (d, J=16.7 Hz, 1H), 7.43 (d, J=8.8 Hz, 1H), 7.48 (s, 1H), 8.63 (br s, 2H), 8.82 (s, 2H), 10.10 (s, 1H). MS (ES+): m/z 497 (M+H)⁺.

### Example 306

### 2-{[5-({5-[(E)-2-Phenylvinyl]pyrimidin-2-yl}amino)-2-(piperazin-1-ylsulfonyl)phenyl]amino}ethanol (Compound CXXXVII)

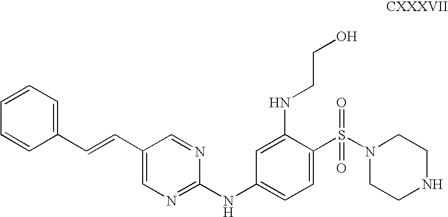

The title compound was isolated as a by-product in the previous example (27.5 mg). Same experimental procedure was used. ¹H NMR (500 MHz, DMSO-d₆): δ 3.15-3.21 (m, 10H), 3.68 (t, J=5.5 Hz, 2H), 6.40 (br s, 1H), 7.17 (d, J=16.6 Hz, 1H), 7.20 (dd, J=8.9, 1.9 Hz, 1H), 7.29 (t, J=7.4 Hz, 1H), 7.34 (d, J=16.6 Hz, 1H), 7.40 (t, J=7.6 Hz, 2H), 7.43 (d, J=8.9 Hz, 1H), 7.49 (d, J=2.0 Hz, 1H), 7.57 (dd, J=8.6, 1.1 Hz, 2H), 8.65 (br s, 2H), 8.83 (s, 2H), 10.11 (s, 1H). MS (ES+): m/z 481 (M+H)⁺.

### Example 307

### 4-Bromo-2-fluoro-N-(2-hydroxyethyl)benzenesulfonamide (Intermediate 168)

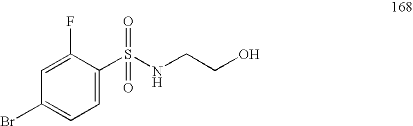

To a solution of ethanolamine (1.11 g, 18.28 mmol) and Et₃N (2.03 g, 20.11 mmol) in 100 mL of DCM was added 4-bromo-2-fluorobenzenesulfonyl chloride (5.0 g, 18.28 mmol) in small portions. The reaction mixture was left to stir at ambient temperature for 3 h. Then it was diluted with ca. 100 mL of EtOAc and washed with DI water (2×200 mL), 0.5 N HCl (2×100 mL), sat. NaHCO₃ (2×100 mL), brine (2×100 mL), dried over anhydrous Na₂SO₄, filtered though a short pad of silica gel and concentrated in vacuo to give the title intermediate as a white solid (4.37 g, 80%).

### Example 308

### tert-Butyl 4-(5-bromo-2-{[(2-hydroxyethyl)amino]sulfonyl}phenyl)piperazine-1-carboxylate (Intermediate 169)

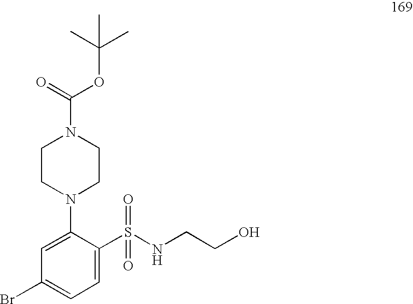

20 mL microwave vial was charged with intermediate 168 (2.0 g, 6.70 mmol), N-Boc piperazine (2.49 g, 13.4 mmol) and anhydrous dioxane (20 mL). The vial was sealed and irradiated in a microwave (Initiator, Biotage) at 150° C. for 20 min. The reaction mixture was diluted with ca. 100 mL of EtOAc and concentrated in vacuo with ca. 15 g of silica gel. The loaded silica gel was taken to the ISCO system for purification (solid method, 40 g column, 0-100% gradient of EtOAc in hexanes, 40 min method). Fractions containing the product were combined and concentrated in vacuo to afford the title intermediate as clear colorless oil (1.45 g, 47%).

### Example 309

### tert-Butyl 4-(2-{[(2-hydroxyethyl)amino]sulfonyl}-5-[(5-vinylpyrimidin-2-yl)amino]phenyl)piperazine-1-carboxylate (Intermediate 170)

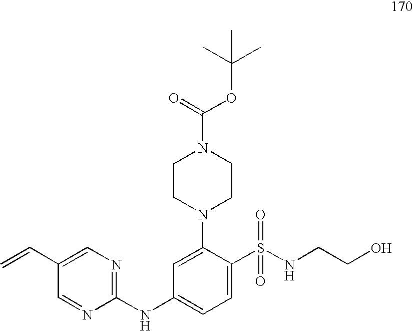

100 mL round-bottom flask was charged with the intermediate 169 (2.65 g, 5.7 mmol), intermediate 1 (829.4 mg, 6.84 mmol), Pd₂(dba)₃ (209.0 mg, 0.228 mmol), Xantphos (396.2 mg, 0.684 mmol), cesium carbonate (3.71 g, 11.41 mmol) and anhydrous dioxane (80 mL). The mixture was purged with argon gas for 30 min, then brought to reflux and refluxed for 3 h under argon atmosphere. After cooling to room temperature, the resulting mixture was diluted with ca. 150 mL of EtOAc and filtered though a short pad of silica gel. The silica gel pad was washed with EtOAc. Combined organic solutions were concentrated in vacuo with ca. 15 g of silica gel. The loaded silica gel was taken to the ISCO system for further purification (80 g column, solid method, 0% to 100% EtOAc gradient in hexanes, 45 min method, 254 nm detection wavelength). Fractions containing the product were combined and concentrated in vacuo to afford the title intermediate as a yellow solid (1.31 g, 46%).

¹H NMR (500 MHz, DMSO-d₆): δ 1.42 (s, 9H), 2.69 (q, J=5.8 Hz, 2H), 2.89 (br s, 4H), 3.36 (q, J=5.5 Hz, 2H), 3.52 (br s, 4H), 4.80 (t, J=5.3 Hz, 1H), 5.27 (d, J=11.4 Hz, 1H), 5.91 (d, J=17.8 Hz, 1H), 6.49 (t, J=6.1 Hz, 1H), 6.64 (dd, J=17.8, 11.3 Hz, 1H), 7.74 (d, J=8.7 Hz, 1H), 7.77 (dd, J=8.8, 1.8 Hz, 1H), 7.97 (d, J=1.6 Hz, 1H), 8.72 (s, 2H), 10.18 (s, 1H). MS (ES+): m/z 405/505 (M+H)⁺.

### Example 310

### 4-({5-[(E)-2-(2-Amino-1,3-benzothiazol-6-yl)vinyl]pyrimidin-2-yl}amino)-N-(2-hydroxyethyl)-2-piperazin-1-ylbenzenesulfonamide (Compound

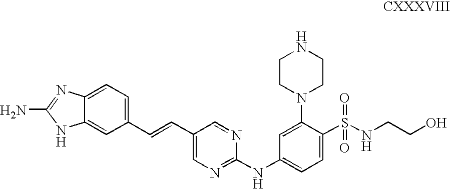

2-5 ml microwave vial was charged with intermediate 170 (504.6 mg, 1.0 mmol), 2-amino-6-bromobenzothiazole (275.0 mg, 1.2 mmol), Pd₂(dba)₃ (91.6 mg, 0.1 mmol), P(t-Bu)₃ (0.4 mL, 0.4 mmol, 1.0 M solution in toluene), cesium carbonate (651.6 mg, 2.0 mmol), Et₃N (2 drops) and anhydrous dioxane (5 mL). The mixture was purged with argon gas for 10 min, then sealed and irradiated in a microwave (Initiator, Biotage) at 180° C. for 2 h. After cooling to room temperature, the cap was removed and the resulting mixture was concentrated in vacuo. The residue was re-dissolved in 5 mL of DMF, filtered though 0.2 micron syringe filter and purified by reverse-phase preparative HPLC in CH₃CN/H₂O system containing 0.05% of TFA. All fractions, containing mono-Boc product, were combined, concentrated in vacuo and treated with 50% TFA in DCM for 10 min. The resulting solution was concentrated in vacuo and the residue was subjected to a second purification by reverse-phase preparative HPLC in CH₃CN/H₂O system containing 0.05% of TFA. Fractions containing the product were combined and poured into ca. 100 mL of saturated aqueous NaHCO₃. The resulting precipitate was collected by centrifugation, washed with water (2×40 mL), MeOH (1×5 mL), Et₂O (2×40 mL) and dried in vacuo to give the title compound as a yellow solid (51.0 mg, 9%).

¹H NMR (500 MHz, DMSO-d₆): δ 2.67 (q, J=5.6 Hz, 2H), 2.82-2.90 (m, 8H), 3.38-3.40 (m, 2H), 4.80 (br s, 1H), 6.52 (t, J=6.2 Hz, 1H), 7.05 (d, J=16.6 Hz, 1H), 7.33 (d, J=16.6 Hz, 1H), 7.33 (d, J=8.3 Hz, 1H), 7.43 (dd, J=8.4, 1.6 Hz, 1H), 7.57 (s, 2H), 7.72 (d, J=8.8 Hz, 1H), 7.78 (dd, J=8.8, 1.7 Hz, 1H), 7.87 (d, J=1.5 Hz, 1H), 7.99 (d, J=1.2 Hz, 1H), 8.80 (s, 2H), 10.19 (s, 1H). MS (ES+): m/z 553 (M+H)⁺.

### Example 311

### N-(2-Hydroxyethyl)-4-({5-[(E)-2-(1H-indazol-4-yl)vinyl]pyrimidin-2-yl}amino)-2-piperazin-1-ylbenzenesulfonamide (Compound CXXXIX)

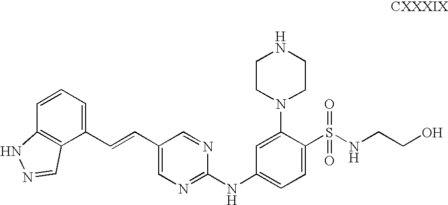

20 microwave vial was charged with intermediate 170 (504.6 mg, 1.0 mmol), 4-bromoindazole (236.4 mg, 1.2 mmol), Pd(OAc)₂ (44.9 mg, 0.2 mmol), PPh₃ (105.0 mg, 0.4 mmol), Et₃N (1.012 g, 1.4 mL, 10.0 mmol) and anhydrous DMF (10 mL). The mixture was purged with argon gas for 10 min, then sealed and irradiated in a microwave (Initiator, Biotage) at 160° C. for 2 h. After cooling to room temperature, the cap was removed and the resulting mixture was filtered though 0.2 micron syringe filter and purified by reverse-phase preparative HPLC in CH₃CN/H₂O system containing 0.05% of TFA. All fractions, containing mono-Boc product, were combined, concentrated in vacuo and treated with 50% TFA in DCM for 10 min. The resulting solution was concentrated in vacuo and the residue was subjected to a second purification by reverse-phase preparative HPLC in CH₃CN/H₂O system containing 0.05% of TFA. Fractions containing the product were combined and dried in vacuo to give the TFA salt of The title compound as a light-yellow solid (26.1 mg, 5%).

¹H NMR (500 MHz, DMSO-d₆): δ 2.71 (q, J=5.9 Hz, 2H), 3.14 (m, 4H), 3.31 (m, 2H), 3.33-3.36 (m, 4H), 4.75 (br s, 1H), 6.72 (t, J=6.2 Hz, 1H), 7.34-7.38 (m, 2H), 7.42 (d, J=16.7 Hz, 1H), 7.48 (d, J=7.1 Hz, 1H), 7.72 (d, J=16.8 Hz, 1H), 7.80 (d, J=8.7, 1H), 7.87-7.91 (m, 2H), 8.59 (s, 1H), 8.83 (br s, 2H), 8.97 (s, 2H), 10.33 (s, 1H), 13.20 (s, 1H). MS (ES+): m/z 521 (M+H)⁺.

### Example 312

### N-(2-Hydroxyethyl)-4-({5-[(E)-2-(3-hydroxyphenyl)vinyl]pyrimidin-2-yl}amino)-2-piperazin-1-ylbenzenesulfonamide (Compound CXL)

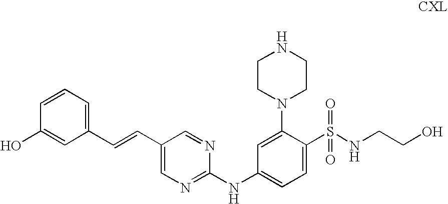

2-5 microwave vial was charged with intermediate 170 (303.0 mg, 0.6 mmol), 3-bromophenol (249.4 mg, 1.44 mmol), Pd(OAc)₂ (26.9 mg, 0.12 mmol), PPh₃ (63.0 mg, 0.24 mmol), Et₃N (607.1 mg, 0.83 mL, 6.0 mmol) and anhydrous DMF (5 mL). The mixture was purged with argon gas for 10 min, then sealed and irradiated in a microwave (Initiator, Biotage) at 160° C. for 30 min. After cooling to room temperature, the cap was removed and the resulting mixture was filtered though 0.2 micron syringe filter and purified by reverse-phase preparative HPLC in CH₃CN/H₂O system containing 0.05% of TFA. All fractions, containing Boc-protected product, were combined, concentrated in vacuo and treated with 50% TFA in DCM for 10 min. The resulting solution was concentrated in vacuo and the residue was subjected to a second purification by reverse-phase preparative HPLC in CH₃CN/H₂O system containing 0.05% of TFA. Fractions containing the product were combined and dried in vacuo to give the TFA salt of The title compound as a bright-yellow solid (34.5 mg, 9%).

¹H NMR (500 MHz, DMSO-d₆): δ 2.71 (q, J=5.9 Hz, 2H), 3.11-3.15 (m, 4H), 3.30 (t, J=5.9 Hz, 2H), 3.34-3.38 (m, 4H), 4.75 (br s, 1H), 6.69-6.72 (m, 2H), 6.96 (s, 1H), 7.01 (d, J=7.9 Hz, 1H), 7.08 (d, J=16.6 Hz, 1H), 7.18 (t, J=7.9 Hz, 1H), 7.25 (d, J=16.6 Hz, 1H), 7.78 (d, J=8.8 Hz, 1H), 7.85 (dd, J=8.9, 1.9 Hz, 1H), 7.90 (d, J=1.8 Hz, 1H), 8.81 (br s, 2H), 8.83 (s, 2H), 10.29 (s, 1H). MS (ES+): m/z 497 (M+H)⁺.

### Example 313

### N-(2-Hydroxyethyl)-4-({5-[(E)-2-phenylvinyl]pyrimidin-2-yl}amino)-2-piperazin-1-ylbenzenesulfonamide (Compound CXLI)

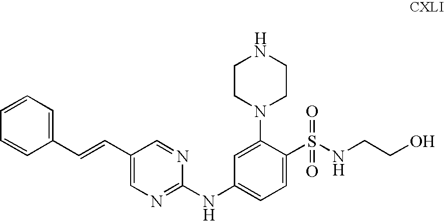

The title compound was isolated as a by-product in the previous example (16.0 mg). The same experimental procedure for isolation and purification was used. ¹H NMR (500 MHz, DMSO-d₆): δ 2.71 (q, J=5.9 Hz, 2H), 3.11-3.15 (m, 4H), 3.30 (t, J=5.9 Hz, 2H), 3.34-3.38 (m, 4H), 4.75 (br s, 1H), 6.72 (t, J=5.8 Hz, 1H), 7.18 (d, J=16.6 Hz, 1H), 7.29 (d, J=7.3 Hz, 1H), 7.34 (d, J=16.7 Hz, 1H), 7.40 (t, J=7.5 Hz, 1H), 7.58 (d, J=7.6 Hz, 1H), 7.78 (d, J=8.6 Hz, 1H), 7.87 (d, J=8.8 Hz, 1H), 7.89 (s, 1H), 8.79 (br s, 2H), 8.83 (s, 2H), 10.30 (s, 1H). MS (ES+): m/z 481 (M+H)⁺.

### Example 314

### tert-Butyl 4-({4-bromo-2-[4-(tert-butoxycarbonyl)piperazin-1-yl]phenyl}sulfonyl)piperazine-1-carboxylate (Intermediate 171)

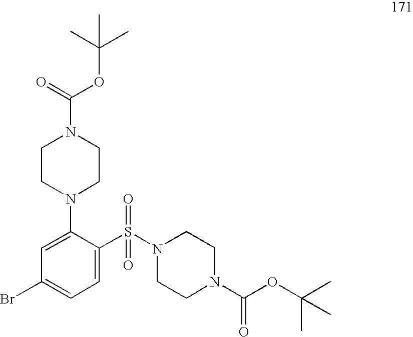

20 mL microwave vial was charged with N-Boc piperazine (1.86 g, 10.0 mmol), intermediate 128 (2.11 g, 5.0 mmol) and anhydrous dioxane (19 mL). Then the vial was sealed and irradiated in a microwave (Initiator, Biotage) at 170° C. for 1 h. After cooling to room temperature, the reaction mixture was diluted with ca. 50 mL of EtOAc and filtered though a short pad of silica gel. Solvent was removed in vacuo to give the title intermediate as a white solid (3.3 g, 94%).

### Example 315

### tert-Butyl 4-(2-{[4-(tert-butoxycarbonyl)piperazin-1-yl]sulfonyl}-5-[(5-vinylpyrimidin-2-yl)amino]phenyl)piperazine-1-carboxylate (Intermediate 172)

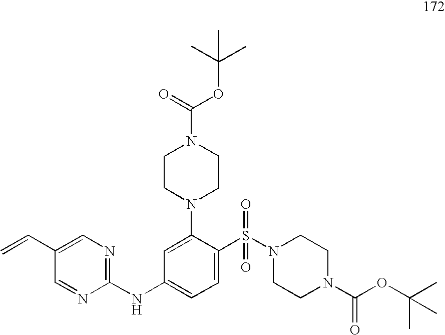

100 mL round-bottom flask was charged with intermediate 171 (3.3 g, 5.6 mmol), the intermediate 1 (746.0 mg, 6.16 mmol), Pd₂(dba)₃ (205.0 mg, 0.22 mmol), Xantphos (388.6 mg, 0.671 mmol), cesium carbonate (3.65 g, 11.2 mmol) and anhydrous dioxane (80 mL). The mixture was purged with argon gas for 30 min, then brought to reflux and refluxed under argon atmosphere for 15 h. After cooling to room temperature, the resulting mixture was diluted with ca. 100 mL of EtOAc and filtered though a short pad of silica gel. The silica gel pad was washed with EtOAc. Combined organic solutions were concentrated in vacuo with ca. 15 g of silica gel. The loaded silica gel was taken to the ISCO system for further purification (80 g column, solid method, 0% to 50% EtOAc gradient in hexanes, 45 min method, 254 nm detection wavelength). Fractions containing the product were combined and concentrated in vacuo to afford the title intermediate as a yellow solid (2.43 g, 69%).

¹H NMR (500 MHz, DMSO-d₆): δ 1.34 (s, 9H), 1.42 (s, 9H), 2.88 (br s, 4H), 2.93 (br s, 4H), 3.32-3.34 (m, 4H), 3.47 (br s, 4H), 5.28 (d, J=11.4 Hz, 1H), 5.93 (d, J=17.9 Hz, 1H), 6.65 (dd, J=17.8, 11.3 Hz, 1H), 7.71 (d, J=8.9 Hz, 1H), 7.74 (dd, J=8.9, 1.9 Hz, 1H), 7.91 (d, J=1.8 Hz, 1H), 8.73 (s, 2H), 10.23 (s, 1H). MS (ES+): m/z 430/530/630 (M+H)⁺.

### Example 316

### N-[3-piperazin-1-yl-4-(piperazin-1-ylsulfonyl)phenyl]-5-vinylpyrimidin-2-amine trifluoroacetic acid salt (Compound CXLII)

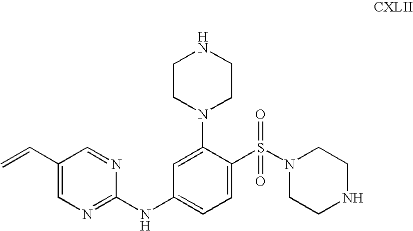

Intermediate 172 (63.0 mg, 0.1 mmol) was treated with 30% TFA in DCM (10 mL) for 10 min and the resulting bright-yellow solution was concentrated in vacuo. The residue was dissolved in 2 mL of DMF, filtered though 0.2 micron syringe filter and purified by reverse-phase preparative HPLC in CH₃CN/H₂O system containing 0.05% of TFA. All fractions containing the product were combined and concentrated in vacuo to give the TFA salt of the title product as a light-yellow solid (60.0 mg, 91%).

¹H NMR (500 MHz, DMSO-d₆): δ 3.12-3.20 (m, 12H), 3.26-3.30 (m, 4H), 5.31 (d, J=11.5 Hz, 1H), 5.93 (d, J=17.9 Hz, 1H), 6.67 (dd, J=17.9, 11.2 Hz, 1H), 7.75 (d, J=9.4 Hz, 1H), 7.87-7.89 (m, 2H), 8.73 (s, 1H), 8.78 (br s, 2H), 8.95 (br s, 2H), 10.36 (1H). MS (ES+): m/z 431 (M+H)⁺.

### Example 317

### 6-[(E)-2-(2-{[3-piperazin-1-yl-4-(piperazin-1-ylsulfonyl)phenyl]amino}pyrimidin-5-yl)vinyl]-1,3-benzothiazol-2-amine (Compound CXLIII)

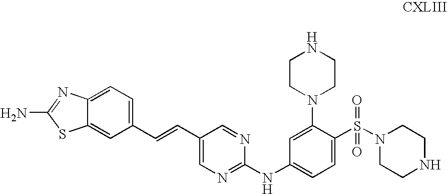

20 ml microwave vial was charged with intermediate 172 (629.7 mg, 1.0 mmol), 2-amino-6-bromobenzothiazole (275.0 mg, 1.2 mmol), Pd₂(dba)₃ (91.6 mg, 0.1 mmol), P(t-Bu)₃ (0.4 mL, 0.4 mmol, 1.0 M solution in toluene), cesium carbonate (651.6 mg, 2.0 mmol), Et₃N (2 drops) and anhydrous dioxane (18 mL). The mixture was purged with argon gas for 20 min, then sealed and irradiated in a microwave (Initiator, Biotage) at 180° C. for 1 h. After cooling to room temperature, the resulting mixture was diluted with ca. 100 mL of EtOAc and filtered though a short pad of silica gel. The silica gel pad was washed with EtOAc. Combined organic solutions were concentrated in vacuo. The residue was dissolved in 5 mL of DMF, filtered though 0.2 micron syringe filter and purified by reverse-phase preparative HPLC in CH₃CN/H₂O system containing 0.05% of TFA. All fractions, containing di-Boc-protected product, were combined, concentrated in vacuo and treated with 50% TFA in DCM for 10 min. The resulting solution was concentrated in vacuo and the residue was subjected to a second purification by reverse-phase preparative HPLC in CH₃CN/H₂O system containing 0.05% of TFA. Fractions containing the product were combined and poured into ca. 100 mL of saturated aqueous NaHCO₃. The resulting precipitate was collected by centrifugation, washed with water (2×40 mL), MeOH (1×5 mL), Et₂O (2×40 mL) and dried in vacuo to give the title compound as a yellow solid (65.0 mg, 11%).

¹H NMR (500 MHz, DMSO-d₆): δ 2.64-2.68 (m, 4H), 2.82-2.88 (m, 14H), 7.05 (d, J=16.6 Hz, 1H), 7.32 (d, J=16.5 Hz, 1H), 7.33 (d, J=8.4 Hz, 1H), 7.44 (dd, J=8.4, 1.6 Hz, 1H), 7.58 (br s, 2H), 7.69 (d, J=8.8 Hz, 1H), 7.74 (dd, J=8.9, 1.9 Hz, 1H), 7.87-7.89 (m, 2H), 8.80 (s, 2H), 10.20 (1H). MS (ES+): m/z 579 (M+H)⁺.

### Example 318

### 5-[(E)-2-(1H-Indazol-4-yl)vinyl]-N-[3-piperazin-1-yl-4-(piperazin-1-ylsulfonyl)phenyl]pyrimidin-2-amine (Compound CXLIV)

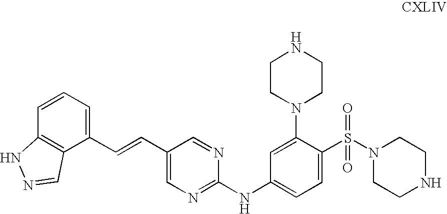

5 microwave vial was charged with intermediate 172 (630.0 mg, 1.0 mmol), 4-bromoindazole (236.4 mg, 1.2 mmol), Pd(OAc)₂ (45.0 mg, 0.2 mmol), PPh₃ (105.0 mg, 0.4 mmol), Et₃N (1.012 g, 1.4 mL, 10.0 mmol) and anhydrous DMF (10 mL). The mixture was purged with argon gas for 20 min, then sealed and placed in a heating block at 140° C. for 18 h. After cooling to room temperature, the cap was removed and the resulting mixture was filtered though 0.2 micron syringe filter and purified by reverse-phase preparative HPLC in CH₃CN/H₂O system containing 0.05% of TFA. All fractions, containing di-Boc-protected product, were combined, concentrated in vacuo and treated with 50% TFA in DCM for 10 min. The resulting solution was concentrated in vacuo and the residue was subjected to a second purification by reverse-phase preparative HPLC in CH₃CN/H₂O system containing 0.05% of TFA. Fractions containing the product were combined and poured into ca. 100 mL of saturated aqueous NaHCO₃. The resulting precipitate was collected by centrifugation, washed with water (2×40 mL), MeOH (1×5 mL), Et₂O (2×40 mL) and dried in vacuo to give the title compound as a light-yellow solid (62.0 mg, 11%).

¹H NMR (500 MHz, DMSO-d₆): δ 2.14 (br s, 1H), 2.65-2.67 (m, 4H), 2.81-2.88 (m, 12H), 3.25 (br s, 1H), 7.33-7.41 (m, 3H), 7.48 (d, J=7.1 Hz, 1H), 7.69-7.49 (m, 3H), 7.94 (br s, 1H), 8.60 (s, 1H), 8.98 (s, 2H), 10.21 (br s, 1H), 13.28 (br s, 1H). MS (ES+): m/z 546 (M+H)⁺.

### Example 319

### 5-[(E)-2-(1H-Indol-4-yl)vinyl]-N-[3-piperazin-1-yl-4-(piperazin-1-ylsulfonyl)phenyl]pyrimidin-2-amine (Compound CXLV)

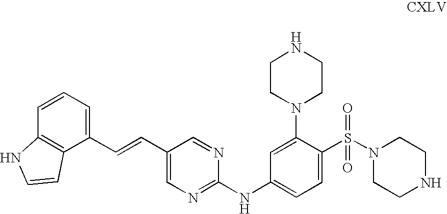

5 microwave vial was charged with intermediate 172 (630.0 mg, 1.0 mmol), 4-bromo-indole-1-carboxylic acid tert-butyl ester (355.4 mg, 1.2 mmol), Pd(OAc)₂ (45.0 mg, 0.2 mmol), PPh₃ (105.0 mg, 0.4 mmol), Et₃N (1.012 g, 1.4 mL, 10.0 mmol) and anhydrous DMF (5 mL). The mixture was purged with argon gas for 20 min, then sealed and irradiated in a microwave (Initiator, Biotage) at 180° C. for 30 min. After cooling to room temperature, the cap was removed and the resulting mixture was filtered though 0.2 micron syringe filter and purified by reverse-phase preparative HPLC in CH₃CN/H₂O system containing 0.05% of TFA. All fractions, containing di-Boc-protected product, were combined, concentrated in vacuo and treated with 30% TFA in DCM for 10 min. The resulting solution was concentrated in vacuo with 2 mL of MeOH and the residue was subjected to a second purification by reverse-phase preparative HPLC in CH₃CN/H₂O system containing 0.05% of TFA. Fractions containing the product were combined and dried in vacuo to give the bis-TFA salt of the title compound as a light-yellow solid (60.0 mg, 8%).

¹H NMR (500 MHz, DMSO-d₆): δ 3.13-3.19 (m, 12H), 3.27-3.31 (m, 4H), 6.91-6.95 (m, 1H), 7.12 (t, J=7.7 Hz, 1H), 7.27 (d, J=16.6 Hz, 1H), 7.32 (d, J=7.2 Hz, 1H), 7.36 (d, J=8.0 Hz, 1H), 7.44 (t, J=2.8 Hz, 1H), 7.70 (d, J=16.7 Hz, 1H), 7.77 (d, J=8.8 Hz, 1H), 7.90-7.95 (m, 2H), 8.72 (br s, 2H), 8.91 (br s, 2H), 8.95 (s, 2H), 10.38 (s, 1H), 11.24 (s, 1H). MS (ES+): m/z 545 (M+H)⁺.

### Example 320

### tert-Butyl 4-({2-fluoro-4-[(5-vinylpyrimidin-2-yl)amino]phenyl}sulfonyl)piperazine-1-carboxylate (Intermediate 173)

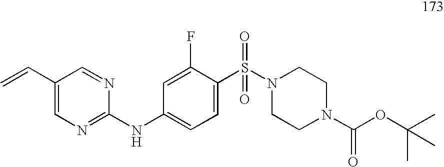

100 mL round-bottom flask was charged with intermediate 128 (4.35 g, 10.27 mmol), intermediate 1 (1.245 g, 10.3 mmol), Pd₂(dba)₃ (377.2 mg, 0.412 mmol), Xantphos (715.2 mg, 1.23 mmol), cesium carbonate (6.71 g, 20.6 mmol) and anhydrous dioxane (80 mL). The mixture was purged with argon gas for 30 min, then brought to reflux and refluxed under argon atmosphere for 24 h. After cooling to room temperature, the resulting mixture was diluted with ca. 150 mL of EtOAc and filtered though a short pad of silica gel. The silica gel pad was washed with EtOAc. Combined organic solutions were concentrated in vacuo with ca. 15 g of silica gel. The loaded silica gel was taken to the ISCO system for further purification (80 g column, solid method, 0% to 100% EtOAc gradient in hexanes, 45 min method, 254 nm detection wavelength). Fractions containing the product were combined and concentrated in vacuo to afford the title intermediate as an off-white solid (3.65 g, 77%).

¹H NMR (500 MHz, DMSO-d₆): δ 1.34 (s, 9H), 2.97 (t, J=4.8 Hz, 4H), 3.39 (t, J=4.8 Hz, 4H), 5.31 (d, J=11.5 Hz, 1H), 5.94 (d, J=17.7 Hz, 1H), 6.66 (dd, J=17.8, 11.3 Hz, 1H), 7.61-7.67 (m, 2H), 7.61-7.67 (m, 2H), 8.05 (dd, J=14.7, 1.8 Hz, 1H), 8.76 (s, 2H), 10.53 (s, 1H). MS (ES+): m/z 364/464 (M+H)⁺.

### Example 321

### 6-[(E)-2-(2-{[3-fluoro-4-(piperazin-1-ylsulfonyl)phenyl]amino}pyrimidin-5-yl)vinyl]-1,3-benzothiazol-2-amine (Compound CXLI)

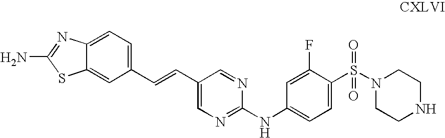

2-5 ml microwave vial was charged with intermediate 173 (232.0 mg, 0.5 mmol), 2-amino-6-bromobenzothiazole (137.5 mg, 0.6 mmol), Pd₂(dba)₃ (45.8 mg, 0.05 mmol), P(t-Bu)₃ (0.2 mL, 0.2 mmol, 1.0 M solution in toluene), cesium carbonate (325.8 mg, 1.0 mmol), Et₃N (2 drops) and anhydrous dioxane (5 mL). The mixture was purged with argon gas for 10 min, then sealed and irradiated in a microwave (Initiator, Biotage) at 180° C. for 1 h. After cooling to room temperature, the cap was removed and the resulting mixture was concentrated in vacuo. The residue was re-dissolved in 5 mL of DMF, filtered though 0.2 micron syringe filter and purified by reverse-phase preparative HPLC in CH₃CN/H₂O system containing 0.05% of TFA. All fractions, containing Boc-protected product, were combined, concentrated in vacuo and treated with 50% TFA in DCM for 10 min. The resulting solution was concentrated in vacuo and the residue was subjected to a second purification by reverse-phase preparative HPLC in CH₃CN/H₂O system containing 0.05% of TFA. Fractions containing the product were combined and poured into ca. 100 mL of saturated aqueous NaHCO₃. The resulting precipitate was collected by centrifugation, washed with water (2×40 mL), MeOH (1×5 mL), Et₂O (2×40 mL) and dried in vacuo to give the title compound as a yellow solid (42.5 mg, 17%).

¹H NMR (500 MHz, DMSO-d₆): δ 2.27 (br s, 1H), 2.69-2.73 (m, 4H), 2.86-2.90 (m, 4H), 7.08 (d, J=16.6 Hz, 1H), 7.34 (d, J=16.6 Hz, 1H), 7.33 (d, J=8.1 Hz, 1H), 7.44 (dd, J=8.4, 1.5 Hz, 1H), 7.58 (s, 2H), 7.62-7.68 (m, 2H), 7.88 (d, J=1.4 Hz, 1H), 8.07 (dd, J=13.7, 1.4 Hz, 1H), 8.83 (s, 2H), 10.52 (s, 1H). MS (ES+): m/z 512 (M+H)⁺.

### Example 322

### tert-Butyl 4-({2-(trifluoromethyl)-4-[(5-vinylpyrimidin-2-yl)amino]phenyl}sulfonyl)piperazine-1-carboxylate (Intermediate 174)

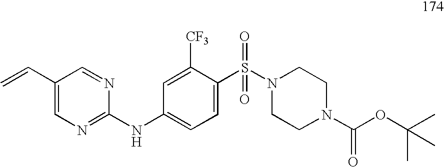

100 mL round-bottom flask was charged with intermediate 126 (4.73 g, 10.0 mmol), the intermediate 1 (1.21 g, 10.0 mmol), Pd₂(dba)₃ (366.3 mg, 0.4 mmol), Xantphos (694.3 mg, 1.2 mmol), cesium carbonate (6.52 g, 20.0 mmol) and anhydrous dioxane (80 mL). The mixture was purged with argon gas for 30 min, then brought to reflux and refluxed under argon atmosphere for 5 h. After cooling to room temperature, the resulting mixture was diluted with ca. 150 mL of EtOAc and filtered though a short pad of silica gel. The silica gel pad was washed with EtOAc. Combined organic solutions were concentrated in vacuo with ca. 15 g of silica gel. The loaded silica gel was taken to the ISCO system for further purification (80 g column, solid method, 0% to 50% EtOAc gradient in hexanes, 45 min method, 254 nm detection wavelength). Fractions containing the product were combined and concentrated in vacuo to afford the title intermediate as a greenish solid (5.1 g, 99%).

¹H NMR (500 MHz, DMSO-d₆): δ 1.36 (s, 9H), 3.09 (t, J=5.0 Hz, 4H), 3.39 (t, J=4.9 Hz, 4H), 5.31 (d, J=11.5 Hz, 1H), 5.96 (d, J=17.8 Hz, 1H), 6.67 (dd, J=17.9, 11.2 Hz, 1H), 7.97 (d, J=9.0 Hz, 1H), 8.28 (dd, J=8.9, 2.2 Hz, 1H), 8.47 (d, J=2.2 Hz, 1H), 8.77 (s, 2H), 10.61 (s, 1H). MS (ES+): m/z 414/514 (M+H)⁺.

### Example 323

### 6-[(E)-2-(2-{[4-(piperazin-1-ylsulfonyl)-3-(trifluoromethyl)phenyl]amino}pyrimidin-5-yl)vinyl]-1,3-benzothiazol-2-amine (Compound CXLVII)

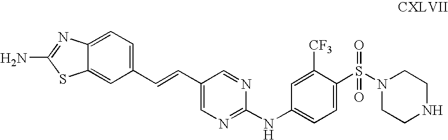

20 ml microwave vial was charged with intermediate 174 (1.027 mg, 2.0 mmol), 6-bromo-benzothiazole (550 mg, 2.4 mmol), Pd₂(dba)₃ (183.1 mg, 0.2 mmol), P(t-Bu)₃ (0.8 mL, 0.8 mmol, 1.0 M solution in toluene), cesium carbonate (1.3 g, 4.0 mmol), and anhydrous dioxane (18 mL). The mixture was purged with argon gas for 10 min, then sealed and irradiated in a microwave (Initiator, Biotage) at 180° C. for 1.5 h. After cooling to room temperature, the resulting mixture was diluted with ca. 100 mL of EtOAc and filtered though a short pad of silica gel. The silica gel pad was washed with EtOAc. Combined organic solutions were concentrated in vacuo with ca. 15 g of silica gel. The loaded silica gel was taken to the ISCO system for further purification (80 g column, solid method, 0% to 100% EtOAc gradient in hexanes, 45 min method, 254 nm detection wavelength). Fractions, containing the Boc-protected product, were combined and concentrated in vacuo to afford the Boc-protected product as a bright-yellow solid (0.434 mg, 32.8%). The solid was treated with 50% TFA in DCM for 10 min. The resulting solution was concentrated in vacuo and the residue was purified by reverse-phase preparative HPLC in CH₃CN/H₂O system containing 0.05% of TFA. Fractions containing the product were combined and poured into ca. 100 mL of saturated aqueous NaHCO₃. The resulting precipitate was collected by centrifugation, washed with water (2×40 mL), MeOH (1×5 mL), Et₂O (2×40 mL) and dried in vacuo to give the title compound as a light-yellow solid (315.0 mg, 73%).

¹H NMR (500 MHz, DMSO-d₆): δ 2.27 (br s, 1H), 2.70 (t, J=4.9 Hz, 4H), 2.98 (t, J=4.9 Hz, 4H), 7.08 (d, J=16.6 Hz, 1H), 7.36 (d, J=16.6 Hz, 1H), 7.33 (d, J=8.2 Hz, 1H), 7.44 (dd, J=8.4, 1.7 Hz, 1H), 7.58 (s, 2H), 7.88 (d, J=1.7 Hz, 1H), 7.97 (d, J=8.9 Hz, 1H), 8.31 (dd, J=8.9, 2.2 Hz, 1H), 8.47 (d, J=2.2 Hz, 1H), 8.84 (s, 2H), 10.60 (s, 1H). MS (ES+): m/z 562 (M+H)⁺.

### Example 324

### tert-Butyl 4-{[(4-bromophenyl)sulfonyl][2-(dimethylamino)ethyl]amino}piperidine-1-carboxylate (Intermediate 175)

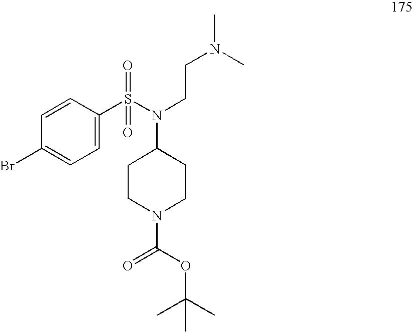

To a solution of tert-butyl 4-{[2-(dimethylamino)ethyl]amino}piperidine-1-carboxylate (2.7 g, 10.0 mmol) and Et₃N (1.01 g, 1.37 mL, 10.0 mmol) in 100 mL of DCM was added 4-bromobenzenesulfonyl chloride (2.55 g, 10.0 mmol). The reaction mixture was stirred at ambient temperature for 4 h. Then it was diluted with ca. 100 mL of EtOAc and washed with DI water (2×200 mL), 0.5 N HCl (2×100 mL), sat. NaHCO₃ (2×100 mL), brine (2×100 mL), dried over anhydrous Na₂SO₄, filtered though a short pad of silica gel and concentrated in vacuo to give the title intermediate as a white solid (4.75 g, 97%). MS (ES+): m/z 491 (M+H)⁺.

### Example 325

### tert-Butyl 4-{[2-(dimethylamino)ethyl]({4-[(5-vinylpyrimidin-2-yl)amino]phenyl}sulfonyl)amino}piperidine-1-carboxylate (Intermediate 176)

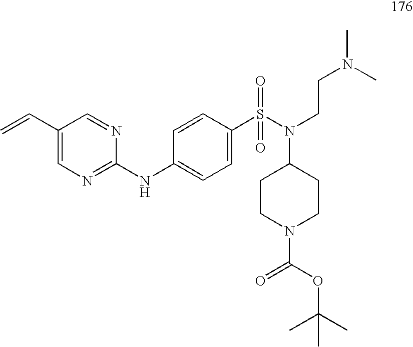

100 mL round-bottom flask was charged with intermediate 175 (4.75 g, 9.68 mmol), intermediate 1 (1.21 g, 10.0 mmol), Pd₂(dba)₃ (366.3 mg, 0.4 mmol), Xantphos (694.3 mg, 1.2 mmol), cesium carbonate (6.52 g, 20.0 mmol) and anhydrous dioxane (80 mL). The mixture was purged with argon gas for 30 min, then brought to reflux and refluxed under argon atmosphere for 18 h. After cooling to room temperature, the resulting mixture was diluted with ca. 150 mL of EtOAc and filtered though a short pad of silica gel. The silica gel pad was washed with 20% MeOH in EtOAc. Combined organic solutions were concentrated in vacuo with ca. 15 g of silica gel. The loaded silica gel was taken to the ISCO system for further purification (80 g column, solid method, 5% to 15% MeOH gradient in DCM with 0.05% Et₃N, 45 min method, 254 nm detection wavelength). Fractions containing the product were combined and concentrated in vacuo to afford the title intermediate as yellow oil (4.47 g, 87%). MS (ES+): m/z 531 (M+H)⁺.

### Example 326

### 4-({5-[(E)-2-(2-Amino-1,3-benzothiazol-6-yl)vinyl}pyrimidin-2-yl]amino)-N-[2-(dimethylamino)ethyl]-N-piperidin-4-ylbenzenesulfonamide (Compound CXLVIII)

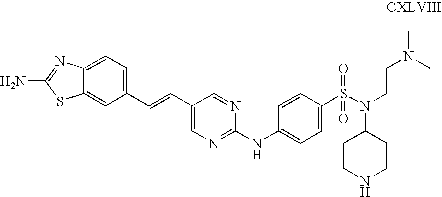

20 ml microwave vial was charged with intermediate 176 (466.0 mg, 0.88 mmol), 2-amino-6-bromobenzothiazole (241.0 mg, 1.05 mmol), Pd₂(dba)₃ (80.6 mg, 0.088 mmol), P(t-Bu)₃ (0.35 mL, 0.35 mmol, 1.0 M solution in toluene), cesium carbonate (573.4 g, 1.76 mmol), and anhydrous dioxane (18 mL). The mixture was purged with argon gas for 20 min, then sealed and irradiated in a microwave (Initiator, Biotage) at 180° C. for 1.5 h. After cooling to room temperature, the cap was removed and the resulting mixture was concentrated in vacuo. The residue was re-dissolved in 8 mL of DMF, filtered though 0.2 micron syringe filter and purified by reverse-phase preparative HPLC in CH₃CN/H₂O system containing 0.05% of TFA. All fractions, containing Boc-protected product, were combined, concentrated in vacuo and treated with 50% TFA in DCM for 10 min. The resulting solution was concentrated in vacuo and the residue was subjected to a second purification by reverse-phase preparative HPLC in CH₃CN/H₂O system containing 0.05% of TFA. Fractions containing the product were combined and poured into ca. 100 mL of saturated aqueous NaHCO₃. The resulting precipitate was collected by centrifugation, washed with water (2×40 mL), MeOH (1×5 mL), Et₂O (2×40 mL) and dried in vacuo to give the title compound as a yellow solid (106.0 mg, 21%).

¹H NMR (500 MHz, DMSO-d₆): δ 1.56 (d, J=11.5 Hz, 2H), 1.79 (dq, J=12.9, 3.6 Hz, 2H), 2.23 (s, 6H), 2.95 (t, J=12.9 Hz, 2H), 3.14-3.18 (m, 4H), 3.23-3.28 (m, 2H), 3.93 (tt, J=12.0, 3.8 Hz, 1H), 7.07 (d, J=16.6 Hz, 1H), 7.32 (d, J=16.6 Hz, 1H), 7.33 (d, J=8.3 Hz, 1H), 7.44 (dd, J=8.4, 1.7 Hz, 1H), 7.58 (s, 2H), 7.78 (d, J=9.0 Hz, 2H), 7.88 (d, J=1.5 Hz, 1H), 8.01 (d, J=8.9 Hz, 2H), 8.80 (s, 2H), 10.31 (s, 1H). MS (ES+): m/z 579 (M+H)⁺.

### Example 327

### N-[2-(Dimethylamino)ethyl]-4-({5-[(E)-2-(1H-indol-4-yl)vinyl]pyrimidin-2-yl}amino)-N-piperidin-4-ylbenzenesulfonamide (Compound CXLIX)

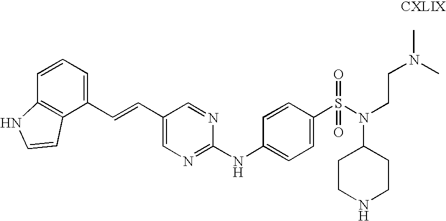

5 microwave vial was charged with intermediate 176 (424.5 mg, 0.8 mmol), 4-bromo-indole-1-carboxylic acid tert-butyl ester (282.3 mg, 0.96 mmol), Pd(OAc)₂ (18.0 mg, 0.08 mmol), PPh₃ (42.0 mg, 0.16 mmol), NaHCO₃ (134.4 mg, 1.6 mmol) and anhydrous DMF (5 mL). The mixture was purged with argon gas for 20 min, then sealed and placed in a heating block at 170° C. for 3 h. After cooling to room temperature, the cap was removed and the resulting mixture was filtered though 0.2 micron syringe filter and purified by reverse-phase preparative HPLC in CH₃CN/H₂O system containing 0.05% of TFA. All fractions, containing Boc-protected product, were combined, concentrated in vacuo and treated with 30% TFA in DCM for 10 min. The resulting solution was concentrated in vacuo with 2 mL of MeOH and the residue was subjected to a second purification by reverse-phase preparative HPLC in CH₃CN/H₂O system containing 0.05% of TFA. Fractions containing the product were combined and poured into ca. 50 mL of saturated aqueous NaHCO₃. The resulting precipitate was collected by centrifugation, washed with water (2×40 mL), MeOH (1×5 mL), Et₂O (2×40 mL) and dried in vacuo to give the title compound as a yellow solid (22 mg, 5%).

¹H NMR (500 MHz, DMSO-d₆): δ 1.30-1.32 (m, 2H), 1.41(dq, J=12.9, 3.6 Hz, 2H), 2.16 (s, 6H), 2.36-2.42 (m, 4H), 2.87 (m, 2H), 3.15 (m, 2H), 3.59 (tt, J=12.0, 3.8 Hz, 1H), 6.94 (m, 1H), 7.12 (t, J=7.7 Hz, 1H), 7.26 (d, J=16.7 Hz, 1H), 7.32 (d, J=7.3 Hz, 1H), 7.35 (d, J=8.0 Hz, 1H), 7.44 (t, J=2.7 Hz, 1H), 7.68 (d, J=16.7 Hz, 1H), 7.75 (d, J=9.0 Hz, 2H), 8.00 (d, J=9.0 Hz, 2H), 8.94 (s, 2H), 10.28 (s, 1H), 11.22 (s, 1H). MS (ES+): m/z 546 (M+H)⁺.

### Example 328

### N-[2-(Dimethylamino)ethyl]-4-({5-[(E)-2-(1H-indazol-4-yl)vinyl]pyrimidin-2-yl}amino)-N-piperidin-4-ylbenzenesulfonamide (Compound CL)

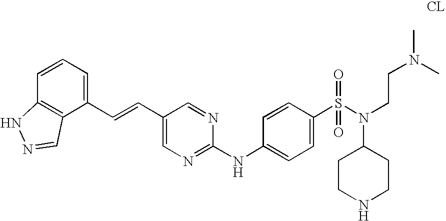

5 microwave vial was charged with intermediate 176 (424.5 mg, 0.8 mmol), intermediate 156 (289.1 mg, 0.96 mmol), Pd(OAc)₂ (18.0 mg, 0.08 mmol), PPh₃ (42.0 mg, 0.16 mmol), NaHCO₃ (134.4 mg, 1.6 mmol) and anhydrous DMF (5 mL). The mixture was purged with argon gas for 20 min, then sealed and placed in a heating block at 170° C. for 8 h. After cooling to room temperature, the cap was removed and the resulting mixture was filtered though 0.2 micron syringe filter and purified by reverse-phase preparative HPLC in CH₃CN/H₂O system containing 0.05% of TFA. All fractions, containing Boc-protected product, were combined, concentrated in vacuo and treated with 30% TFA in DCM for 10 min. The resulting solution was concentrated in vacuo and the residue was subjected to a second purification by reverse-phase preparative HPLC in CH₃CN/H₂O system containing 0.05% of TFA. Fractions containing the product were combined and poured into ca. 50 mL of saturated aqueous NaHCO₃. The resulting precipitate was collected by centrifugation, washed with water (2×40 mL), MeOH (1×5 mL), Et₂O (2×40 mL) and dried in vacuo to give the title compound as a yellow solid (55 mg, 13%).

¹H NMR (500 MHz, DMSO-d₆): δ 1.30-1.32 (m, 2H), 1.41(dq, J=12.9, 3.6 Hz, 2H), 2.16 (s, 6H), 2.36-2.43 (m, 4H), 2.85-2.89 (m, 2H), 3.12-3.17 (m, 2H), 3.59 (tt, J=12.0, 3.8 Hz, 1H), 7.32-7.37 (m, 2H), 7.41 (d, J=16.7 Hz, 1H), 7.46-7.48 (m, 1H), 7.70 (d, J=16.6 Hz, 1H), 7.45 (d, J=8.9 Hz, 2H), 8.00 (d, J=8.9 Hz, 2H), 8.60 (s, 1H), 8.97 (s, 2H), 10.33 (s, 1H), 13.20 (s, 1H). MS (ES+): m/z 547 (M+H)⁺.

### Example 329

### tert-Butyl {1-[(4-bromophenyl)sulfonyl]piperidin-4-yl}carbamate (Intermediate 177)

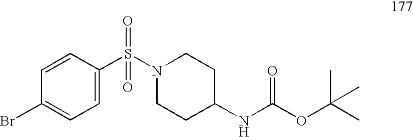

To a solution of tert-butyl piperidin-4-ylcarbamate (5.0 g, 24.96 mmol) and Et₃N (2.53 g, 3.48 mL, 25.0 mmol) in 100 mL of DCM was added 4-bromobenzenesulfonyl chloride (6.4 g, 25.0 mmol). The reaction mixture was stirred at ambient temperature for 4 h. Then it was diluted with ca. 100 mL of EtOAc and washed with DI water (2×200 mL), 0.5 N HCl (2×100 mL), sat. NaHCO₃ (2×100 mL), brine (2×100 mL), dried over anhydrous Na₂SO₄, filtered though a short pad of silica gel and concentrated in vacuo to give the title intermediate as a white solid (10.25 g, 98%). MS (ES+): m/z 420 (M+H)⁺.

### Example 330

### tert-Butyl [1-({4-[(5-vinylpyrimidin-2-yl)amino]phenyl}sulfonyl)piperidin-4-yl]carbamate (Intermediate 178)

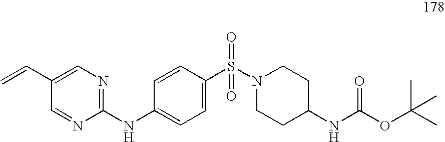

100 mL round-bottom flask was charged with intermediate 1 (605.0 mg, 5.0 mmol), intermediate 177 (2.1 g, 5.0 mmol), Pd₂(dba)₃ (183.1 mg, 0.2 mmol), Xantphos (347.1 mg, 0.6 mmol), cesium carbonate (3.25 g, 10.0 mmol) and 80 mL of anhydrous dioxane. The reaction mixture was purged with the argon gas for 30 min; then it was heated to reflux and refluxed under argon atmosphere for 7 h. The completion of the reaction was monitored by TLC and LC/MS. Upon completion the reaction mixture was cooled down to ambient temperature, diluted with ca. 100 mL of EtOAc and filtered though a short pad of silica gel. The silica gel pad was washed with EtOAc. Combined organic solutions were concentrated in vacuo with ca. 15 g of silica gel. The loaded silica gel was taken to the ISCO system for further purification (80 g column, solid method, 0% to 100% EtOAc gradient in hexanes, 40 min method). Fractions containing the product were combined and concentrated in vacuo to give a yellow solid (1.97 g, 86%). The solid were re-crystallized from 10 mL of EtOAc to give the title intermediate as a fine white powder (1.72 g, 75%).

¹H NMR (500 MHz, DMSO-d₆): δ 1.34 (s, 9H), 1.34-1.43 (m, 2H), 1.74-1.76 (m, 2H), 2.39 (t, J=10.7 Hz, 2H), 3.21 (br s, 1H), 3.41-3.45 (m, 2H), 7.07 (d, J=16.6 Hz, 1H), 5.29 (d, J=11.3 Hz, 1H), 5.92 (d, J=17.9 Hz, 1H), 6.66 (dd, J=17.9, 11.2 Hz, 1H), 6.84 (d, J=7.1 Hz, 1H), 7.64 (d, J=8.9 Hz, 2H), 8.02 (d, J=8.9 Hz, 2H), 8.72 (s, 2H), 10.31 (s, 1H). MS (ES+): m/z 460 (M+H)⁺.

### Example 331

### 6-{(E)-2-[2-({4-[(4-Aminopiperidin-1-yl)sulfonyl]phenyl}amino)pyrimidin-5-yl]vinyl}-1,3-benzothiazol-2-amine (Compound CLI)

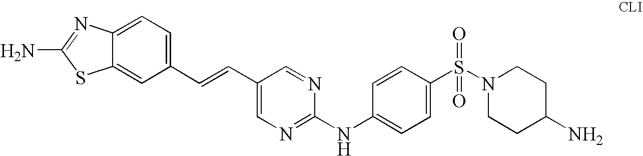

20 ml microwave vial was charged with intermediate 178 (919.1 mg, 2.0 mmol), 2-amino-6-bromobenzothiazole (550 mg, 2.4 mmol), Pd₂(dba)₃ (183.1 mg, 0.2 mmol), P(t-Bu)₃ (0.8 mL, 0.8 mmol, 1.0 M solution in toluene), cesium carbonate (1.3 g, 4.0 mmol), and anhydrous dioxane (18 mL). The mixture was purged with argon gas for 20 min, then sealed and irradiated in a microwave (Initiator, Biotage) at 180° C. for 1.5 h. After cooling to room temperature, the resulting mixture was diluted with ca. 100 mL of EtOAc and filtered though a short pad of silica gel. The silica gel pad was washed with 10% MeOH in EtOAc. Combined organic solutions were concentrated in vacuo with ca. 15 g of silica gel. The loaded silica gel was taken to the ISCO system for further purification (80 g column, solid method, 0% to 100% EtOAc gradient in hexanes, 45 min method, 254 nm detection wavelength). Fractions, containing the Boc-protected product, were combined and concentrated in vacuo to afford the Boc-protected product as a bright-yellow solid (430.0 mg, 35%). The solid was treated with 50% TFA in DCM for 10 min. The resulting solution was concentrated in vacuo and the residue was purified by reverse-phase preparative HPLC in CH₃CN/H₂O system containing 0.05% of TFA. Fractions containing the product were combined and poured into ca. 150 mL of saturated aqueous NaHCO₃. The resulting precipitate was collected by centrifugation, washed with water (2×40 mL), MeOH (1×5 mL), Et₂O (2×40 mL) and dried in vacuo to give the title compound as a light-yellow solid (315.0 mg, 30%).

¹H NMR (500 MHz, DMSO-d₆): δ 1.21-1.28 (m, 2H), 1.70-1.72 (m, 2H), 2.33-2.37 (m, 2H), 2.50-2.54 (m, 1H), 3.41-3.43 (m, 2H), 7.07 (d, J=16.6 Hz, 1H), 7.32 (d, J=16.6 Hz, 1H), 7.33 (d, J=8.2 Hz, 1H), 7.44 (dd, J=8.4, 1.3 Hz, 1H), 7.57 (s, 2H), 7.64 (d, J=8.9 Hz, 2H), 7.88 (s, 1H), 8.02 (d, J=8.8 Hz, 2H), 8.80 (s, 2H), 10.30 (s, 1H). MS (ES+): m/z 508 (M+H)⁺.

### Example 332

### N-{4-[(4-Aminopiperidin-1-yl)sulfonyl]phenyl}-5-[(E)-2-(1H-indazol-4-yl)vinyl]pyrimidin-2-amine (Compound CLII)

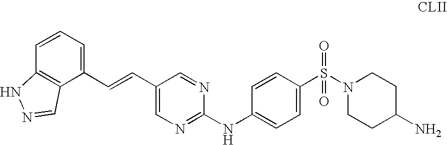

5 microwave vial was charged with intermediate 178 (460.0 mg, 1.0 mmol), intermediate 156 (361.4 mg, 1.2 mmol), Pd(OAc)₂ (22.5 mg, 0.1 mmol), PPh₃ (52.4 mg, 0.2 mmol), NaHCO₃ (168 mg, 2.0 mmol) and anhydrous DMF (5 mL). The mixture was purged with argon gas for 20 min, then sealed and placed in a heating block at 170° C. for 4 h. After cooling to room temperature, the cap was removed and the resulting mixture was filtered though 0.2 micron syringe filter and purified by reverse-phase preparative HPLC in CH₃CN/H₂O system containing 0.05% of TFA. All fractions containing Boc-protected product were combined, concentrated in vacuo and treated with 30% TFA in DCM for 10 min. The resulting solution was concentrated in vacuo and the residue was subjected to a second purification by reverse-phase preparative HPLC in CH₃CN/H₂O system containing 0.05% of TFA. Fractions containing the product were combined and poured into ca. 50 mL of saturated aqueous NaHCO₃. The resulting precipitate was collected by centrifugation, washed with water (2×40 mL), MeOH (1×5 mL), Et₂O (2×40 mL) and dried in vacuo to give the title compound as a white solid (44 mg, 9%).

¹H NMR (500 MHz, DMSO-d₆): δ 1.21-1.28 (m, 2H), 1.70-1.72 (m, 2H), 2.33-2.37 (m, 2H), 2.50-2.54 (m, 1H), 3.41-3.43 (m, 2H), 7.34-7.38 (m, 2H), 7.41 (d, J=16.7 Hz, 1H), 7.47-7.48 (m, 1H), 7.65 (d, J=8.9 Hz, 2H), 7.71 (d, J=16.8 Hz, 1H), 8.03 (d, J=8.9 Hz, 2H), 8.60 (s, 1H), 8.97 (s, 2H), 10.37 (s, 1H), 13.20 (s, 1H). MS (ES+): m/z 476 (M+H)⁺.

### Example 333

### N-{4-[(4-Aminopiperidin-1-yl)sulfonyl]phenyl}-5-[(E)-2-(1H-indol-4-yl)vinyl]pyrimidin-2-amine (Compound CLIII)

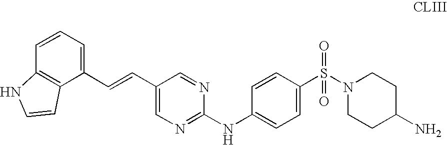

5 microwave vial was charged with intermediate 178 (460.0 mg, 1.0 mmol), 4-bromo-indole-1-carboxylic acid tert-butyl ester (355.4 mg, 1.2 mmol), Pd(OAc)₂ (22.5 mg, 0.1 mmol), PPh₃ (52.4 mg, 0.2 mmol), NaHCO₃ (168 mg, 2.0 mmol) and anhydrous DMF (5 mL). The mixture was purged with argon gas for 20 min, then sealed and placed in a heating block at 160° C. for 4 h. After cooling to room temperature, the cap was removed and the resulting mixture was filtered though 0.2 micron syringe filter and purified by reverse-phase preparative HPLC in CH₃CN/H₂O system containing 0.05% of TFA. All fractions, containing Boc-protected product, were combined, concentrated in vacuo and treated with 30% TFA in DCM for 10 min. The resulting solution was concentrated in vacuo with 2 mL of MeOH and the residue was subjected to a second purification by reverse-phase preparative HPLC in CH₃CN/H₂O system containing 0.05% of TFA. Fractions containing the product were combined and poured into ca. 50 mL of saturated aqueous NaHCO₃. The resulting precipitate was collected by centrifugation, washed with water (2×40 mL), MeOH (1×5 mL), Et₂O (2×40 mL) and dried in vacuo to give the title compound as a yellow solid (21 mg, 4%).

¹H NMR (500 MHz, DMSO-d₆): δ 1.21-1.28 (m, 2H), 1.45 (br s, 2H), 1.70-1.73 (m, 2H), 2.33-2.37 (m, 2H), 2.50-2.54 (m, 1H), 3.41-3.43 (m, 2H), 6.93-6.95 (m, 1H), 7.12 (t, J=7.7 Hz, 1H), 7.26 (d, J=16.6 Hz, 1H), 7.32 (d, J=7.3 Hz, 1H), 7.38 (d, J=8.0, 1H), 7.44 (t, J=2.8 Hz, 1H), 7.65 (d, J=8.9 Hz, 2H), 7.68 (d, J=16.7 Hz, 1H), 8.02 (d, J=8.9 Hz, 2H), 8.94 (s, 2H), 10.32 (s, 1H), 11.22 (s, 1H). MS (ES+): m/z 475 (M+H)⁺.

### Example 334

### tert-Butyl 4-{[(4-bromophenyl)sulfonyl](2-methoxy-2-oxoethyl)amino}piperidine-1-carboxylate (Intermediate 179)

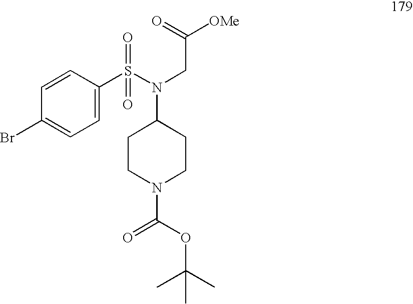

To a solution of intermediate 12 (6.0 g, 14.3 mmol) and methyl bromoacetate (4.82 g, 31.48 mmol) in 100 mL of CH₃CN was added solid Cs₂CO₃ (9.32 g, 28.6 mmol). The reaction mixture was stirred at ambient temperature for 6 h. Then it was filtered though a short pad of silica gel. The silica gel pad was washed with EtOAc. Combined organic solution was concentrated in vacuo with ca. 15 g of silica gel. The loaded silica gel was taken to the ISCO system for purification (80 g column, 45 min method, 0 to 100% gradient of EtOAc in hexanes, 254 nm detection wavelength). Fractions containing the product were combined and solvent was removed in vacuo to give the title intermediate as a white solid (6.63 g, 88%). MS (ES+): m/z 492 (M+H)⁺.

### Example 335

### tert-Butyl 4-1[(4-bromophenyl)sulfonyl](2-hydroxyethyl)amino]piperidine-1-carboxylate (Intermediate 180)

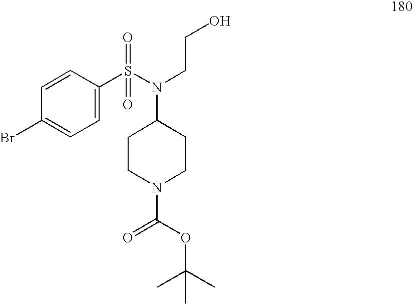

To a solution of intermediate 179 (3.63 g, 7.39 mmol) in 50 mL of anhydrous THF was added solid LiAlH₄ (95%, 590 mg, 14.77 mmol) in small portions. The reaction mixture was stirred at ambient temperature for 2 h. 10 mL of EtOAc was added to quench the reaction and the mixture was stirred for 30 min. Then it was poured into ca. 200 mL of H₂O and the resulting mixture was extracted with EtOAc (4×100 mL). Combined organic extracts were washed with brine (2×100 mL), dried over anhydrous Na₂SO₄, filtered though a short pad of silica gel and concentrated in vacuo to give the title intermediate as an off-white oil (3.40 g, 99%). MS (ES+): m/z 464 (M+H)⁺.

### Example 336

### tert-Butyl 4-[(2-hydroxyethyl)({4-[(5-vinylpyrimidin-2-yl)amino]phenyl}sulfonyl)amino]piperidine-1-carboxylate (Intermediate 181)

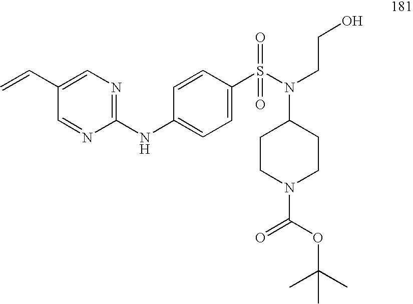

100 mL round-bottom flask was charged with intermediate 1 (888.0 mg, 7.34 mmol), intermediate 180 (3.40 g, 7.34 mmol), Pd₂(dba)₃ (268.7 mg, 0.293 mmol), Xantphos (510.0 mg, 0.88 mmol), cesium carbonate (4.78 g, 14.78 mmol) and 80 mL of anhydrous dioxane. The reaction mixture was purged with the argon gas for 30 min; then it was heated to reflux and refluxed under argon atmosphere for 6 h. The completion of the reaction was monitored by TLC and LC/MS. Upon completion the reaction mixture was cooled down to ambient temperature, diluted with ca. 100 mL of EtOAc and filtered though a short pad of silica gel. The silica gel pad was washed with EtOAc. Combined organic solutions were concentrated in vacuo with ca. 15 g of silica gel. The loaded silica gel was taken to the ISCO system for further purification (80 g column, solid method, 0% to 100% EtOAc gradient in hexanes, 40 min method). Fractions containing the product were combined and concentrated in vacuo to give the title intermediate as a yellow solid (3.05 g, 83%).

¹H NMR (500 MHz, DMSO-d₆): δ 1.36 (s, 9H), 1.32-1.37 (m, 2H), 1.41 (dq, J=12.0, 3.8 Hz, 2H), 2.69 (br s, 2H), 3.09 (t, J=7.1 Hz, 2H), 3.47 (q, J=6.9 Hz, 2H), 3.74 (tt, J=11.8, 3.9 Hz, 1H), 3.91 (br s, 2H), 4.72 (t, J=5.8 Hz, 1H), 5.28 (d, J=11.6 Hz, 1H), 5.91 (d, J=17.8 Hz, 1H), 6.66 (dd, J=17.8, 11.3 Hz, 1H), 7.74 (d, J=8.9 Hz, 2H), 7.97 (d, J=8.9 Hz, 2H), 8.71 (s, 2H), 10.28 (s, 1H). MS (ES+): m/z 504 (M+H)⁺.

### Example 337

### 4-({5-[(E)-2-(2-Amino-1,3-benzothiazol-6-yl)vinyl]pyrimidin-2-yl}amino)-N-(2-hydroxyethyl)-N-piperidin-4-ylbenzenesulfonamide (Compound CLIV)

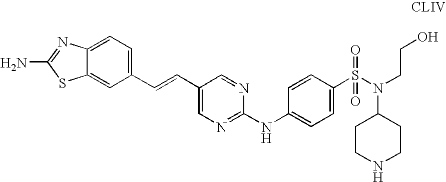

20 ml microwave vial was charged with intermediate 181 (1.00 mg, 2.0 mmol), 2-amino-6-bromobenzothiazole (550 mg, 2.4 mmol), Pd₂(dba)₃ (183.1 mg, 0.2 mmol), P(t-Bu)₃ (0.8 mL, 0.8 mmol, 1.0 M solution in toluene), cesium carbonate (1.3 g, 4.0 mmol), and anhydrous dioxane (18 mL). The mixture was purged with argon gas for 20 min, then sealed and irradiated in a microwave (Initiator, Biotage) at 180° C. for 1.5 h. After cooling to room temperature, the resulting mixture was diluted with ca. 100 mL of EtOAc and filtered though a short pad of silica gel. The silica gel pad was washed with 10% MeOH in EtOAc. Combined organic solutions were concentrated in vacuo with ca. 15 g of silica gel. The loaded silica gel was taken to the ISCO system for further purification (80 g column, solid method, 0% to 10% MeOH gradient in EtOAc, 45 min method, 254 nm detection wavelength). Fractions, containing the Boc-protected product, were combined and concentrated in vacuo to afford the Boc-protected product as a bright-yellow solid (725.0 mg, 55%). The solid was treated with 50% TFA in DCM for 10 min. The resulting solution was concentrated in vacuo and the residue was purified by reverse-phase preparative HPLC in CH₃CN/H₂O system containing 0.05% of TFA. Fractions containing the product were combined and poured into ca. 200 mL of saturated aqueous NaHCO₃. The resulting precipitate was collected by centrifugation, washed with water (2×40 mL), MeOH (1×5 mL), Et₂O (2×40 mL) and dried in vacuo to give the title compound as a pale-yellow solid (490 mg, 44%).

¹H NMR (500 MHz, DMSO-d₆): δ 1.27-1.29 (m, 2H), 1.41 (dq, J=12.0, 3.8 Hz, 2H), 2.38 (t, J=11.6 Hz, 2H), 2.87 (d, J=12.0 Hz, 2H), 3.12 (t, J=7.4 Hz, 2H), 3.50 (t, J=7.2 Hz, 2H), 3.55-3.60 (m, 1H), 4.76 (br s, 1H), 7.07 (d, J=16.6 Hz, 1H), 7.31 (d, J=16.6 Hz, 1H), 7.33 (d, J=8.3 Hz, 1H), 7.44 (dd, J=8.5, 1.6 Hz, 1H), 7.58 (s, 2H), 7.73 (d, J=8.9 Hz, 2H), 7.88 (d, J=1.5 Hz, 1H), 7.98 (d, J=8.9 Hz, 2H), 8.79 (s, 2H), 10.27 (s, 1H). MS (ES+): m/z 552 (M+H)⁺.

### Example 338

### N-(2-Hydroxyethyl)-4-({5-[(E)-2-(1H-indazol-4-yl)vinyl]pyrimidin-2-yl}amino)-N-piperidin-4-ylbenzenesulfonamide (Compound CLV)

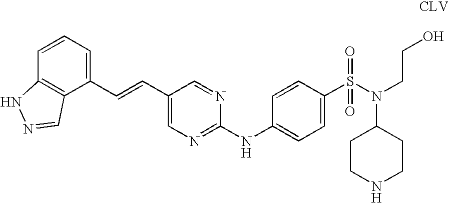

5 microwave vial was charged with intermediate 181 (503.6 mg, 1.0 mmol), intermediate 156 (361.4 mg, 1.2 mmol), Pd(OAc)₂ (22.5 mg, 0.1 mmol), PPh₃ (52.4 mg, 0.2 mmol), NaHCO₃ (168 mg, 2.0 mmol) and anhydrous DMF (5 mL). The mixture was purged with argon gas for 20 min, then sealed and placed in a heating block at 160° C. for 18 h. The build-up excess pressure was ° C.casionally released via a needle. After cooling to room temperature, the cap was removed and the resulting mixture was filtered though 0.2 micron syringe filter and purified by reverse-phase preparative HPLC in CH₃CN/H₂O system containing 0.05% of TFA. All fractions containing the Boc-protected product were combined, concentrated in vacuo and treated with 50% TFA in DCM for 10 min. The resulting solution was concentrated in vacuo with 2 mL of MeOH and the residue was subjected to a second purification by reverse-phase preparative HPLC in CH₃CN/H₂O system containing 0.05% of TFA. Fractions containing the product were combined and passed though Stratospheres PL-HCO3 MP SPE cartridge (200 mg). Solvent was removed in vacuo to give the title compound as a beige solid (38.5 mg, 7%).

¹H NMR (500 MHz, DMSO-d₆): δ 1.29-1.31 (m, 2H), 1.37-1.46 (dq, J=12.0, 3.8 Hz, 2H), 2.41 (t, J=11.3 Hz, 2H), 2.89 (d, J=12.0 Hz, 2H), 3.12 (t, J=7.4 Hz, 2H), 3.48-3.52 (m, 2H), 3.60 (tt, J=11.8, 3.9 Hz, 1H), 4.77 (br s, 1H), 7.34-7.38 (m, 2H), 7.41 (d, J=16.7 Hz, 1H), 7.46-7.48 (m, 1H), 7.71 (d, J=16.7 Hz, 1H), 7.75 (d, J=9.0 Hz, 2H), 8.00 (d, J=8.9 Hz, 2H), 8.60 (s, 1H), 8.97 (s, 2H), 10.34 (s, 1H), 13.20 (s, 1H). MS (ES+): m/z 520 (M+H)⁺.

### Example 339

### N-(2-Hydroxyethyl)-4-({5-[(E)-2-(1H-indol-4-yl)vinyl]pyrimidin-2-yl}amino)-N-piperidin-4-ylbenzenesulfonamide (Compound CLVI)

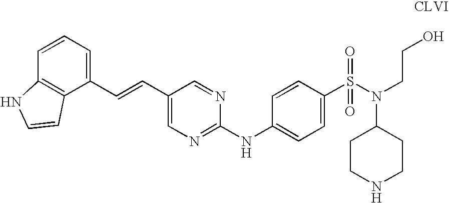

5 microwave vial was charged with intermediate 181 (503.6 mg, 1.0 mmol), 4-bromo-indole-1-carboxylic acid tert-butyl ester (355.4 mg, 1.2 mmol), Pd(OAc)₂ (22.5 mg, 0.1 mmol), PPh₃ (52.4 mg, 0.2 mmol), NaHCO₃ (168 mg, 2.0 mmol) and anhydrous DMF (5 mL). The mixture was purged with argon gas for 20 min, then sealed and placed in a heating block at 160° C. for 4 h. The build-up excess pressure was occasionally released via a needle. After cooling to room temperature, the cap was removed and the resulting mixture was filtered though 0.2 micron syringe filter and purified by reverse-phase preparative HPLC in CH₃CN/H₂O system containing 0.05% of TFA. All fractions, containing Boc-protected product, were combined, concentrated in vacuo and treated with 50% TFA in DCM for 10 min. The resulting solution was concentrated in vacuo with 2 mL of MeOH and the residue was subjected to a second purification by reverse-phase preparative HPLC in CH₃CN/H₂O system containing 0.05% of TFA. Fractions containing the product were combined and passed though Stratospheres PL-HCO₃ MP SPE cartridge (200 mg). Solvent was removed in vacuo to give the title compound as a tan solid (39.3 mg, 8%).

¹H NMR (500 MHz, DMSO-d₆): δ 1.28-1.30 (m, 2H), 1.40 (dq, J=11.9, 3.9 Hz, 2H), 2.38 (t, J=11.3 Hz, 2H), 2.89 (d, J=12.0 Hz, 2H), 3.12 (t, J=7.4 Hz, 2H), 3.47-3.52 (m, 2H), 3.58 (tt, J=11.8, 3.9 Hz, 1H), 4.77 (br s, 1H), 6.94 (m, 1H), 7.12 (t, J=7.7 Hz, 1H), 7.26 (d, J=16.6 Hz, 1H), 7.32 (d, J=7.4 Hz, 1H), 7.35 (d, J=8.0, 1H), 7.44 (t, J=2.8 Hz, 1H), 7.65 (d, J=8.9 Hz, 2H), 7.68 (d, J=16.7 Hz, 1H), 7.74 (d, J=8.9 Hz, 2H), 8.0 (d, J=9.0 Hz, 2H), 8.93 (s, 2H), 10.29 (s, 1H), 11.22 (s, 1H). MS (ES+): m/z 519 (M+H)⁺.

### Example 340

### tert-Butyl 4-({[4-({5-[(E)-2-(2-amino-1,3-benzothiazol-6-yl)vinyl]pyrimidin-2-yl}amino)phenyl]sulfonyl}amino)piperidine-1-carboxylate (Intermediate 182)

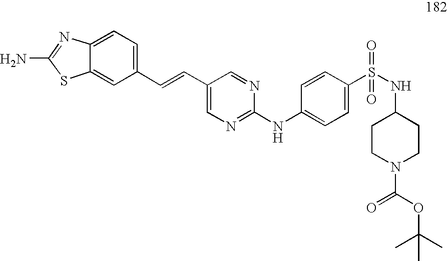

20 ml microwave vial was charged with intermediate 60 (460 mg, 1.0 mmol), 2-amino-6-bromobenzothiazole (275.0 mg, 1.2 mmol), Pd₂(dba)₃ (91.6 mg, 0.1 mmol), P(t-Bu)₃ (0.4 mL, 0.4 mmol, 1.0 M solution in toluene), cesium carbonate (651 mg, 2.0 mmol), and anhydrous dioxane (18 mL). The mixture was purged with argon gas for 20 min, then sealed and irradiated in a microwave (Initiator, Biotage) at 180° C. for 1 h. After cooling to room temperature, the resulting mixture was diluted with ca. 100 mL of EtOAc and filtered though a short pad of silica gel. The silica gel pad was washed with 10% MeOH in EtOAc. Combined organic solutions were concentrated in vacuo with ca. 10 g of silica gel. The loaded silica gel was taken to the ISCO system for further purification (40 g column, solid method, 0% to 100% EtOAc gradient in hexanes, 25 min method, 350 nm detection wavelength). Fractions containing the product were combined and concentrated in vacuo to afford the title intermediate as a yellow solid (226 mg, 37%).

### Example 341

### N²-{[4-({5-[(E)-2-(2-Amino-1,3-benzothiazol-6-yl)vinyl]pyrimidin-2-yl}amino)phenyl]sulfonyl}-N-2-piperidin-4-ylglycinamide (Compound CLVII)

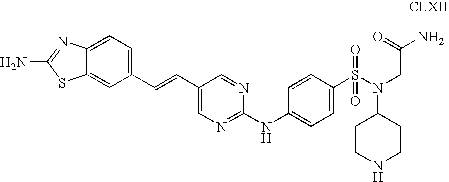

To a solution of intermediate 182 (60.8 mg, 0.1 mmol) in 2 mL of anhydrous DMF was added cesium carbonate (65.5 mg, 0.2 mmol), followed by iodoacetamide (44.4 mg, 0.24 mmol). The reaction mixture was stirred at ambient temperature for 3 h. Then it was filtered though 0.2 micron syringe filter and purified by reverse-phase preparative HPLC in CH₃CN/H₂O system containing 0.05% of TFA. All fractions containing Boc-protected product were combined, concentrated in vacuo and treated with 50% TFA in DCM for 10 min. The resulting solution was concentrated in vacuo and the residue was subjected to a second purification by reverse-phase preparative HPLC in CH₃CN/H₂O system containing 0.05% of TFA. Fractions containing the product were combined and passed though Stratospheres PL-HCO₃ MP SPE cartridge (200 mg). Solvent was removed in vacuo to give the title compound as a pale-yellow solid (27.0 mg, 48%).

¹H NMR (500 MHz, DMSO-d₆): δ 1.27-1.36 (m, 4H), 1.81 (br s, 1H), 2.34 (t, J=10.7 Hz, 2H), 2.84 (d, J=12.1 Hz, 2H), 3.52-3.57 (m, 1H), 3.70 (s, 1H), 7.07 (d, J=16.6 Hz, 1H), 7.08 (br s, 1H), 7.16 (br s, 1H), 7.31 (d, J=16.5 Hz, 1H), 7.33 (d, J=8.3, 1H), 7.44 (dd, J=8.4, 1.6 Hz, 1H), 7.57 (s, 2H), 7.83 (d, J=9.0 Hz, 2H), 7.88 (d, J=1.4 Hz, 1H), 7.97 (d, J=9.0 Hz, 2H), 8.79 (s, 2H), 10.26 (s, 1H). MS (ES+): m/z 565 (M+H)⁺.

### Example 342

### Methyl N-(1-{[4-({5-[(E)-2-(2-amino-1,3-benzothiazol-6-yl)vinyl]pyrimidin-2-yl}amino)phenyl]sulfonyl}piperidin-4-yl)glycinate (Intermediate 183)

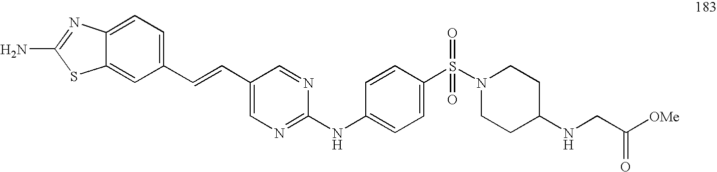

To a solution of the above described compound CLI (360.0 mg, 0.217 mmol) in 5 mL of anhydrous DMF was added cesium carbonate (231.0 mg, 0.709 mmol), followed by a solution of methyl bromoacetate (108.5 mg, 0.709 mmol) in 2 mL of anhydrous DMF. The reaction mixture was left to stir at ambient temperature for 2 h. Then it was filtered though 0.2 micron syringe filter and purified by reverse-phase preparative HPLC in CH₃CN/H₂O system containing 0.05% of TFA. All fractions containing the product were combined and poured into ca. 100 mL of EtOAc. The resulting solution was washed with sat. NaHCO₃ (2×100 mL), brine (2×100 mL), dried over anhydrous Na₂SO₄, filtered and concentrated in vacuo to give the title intermediate as a light-yellow solid (223.0 mg, 54%).

### Example 343

### 2-[(1-{[4-({5-[(E)-2-(2-Amino-1,3-benzothiazol-6-yl)vinyl]pyrimidin-2-yl}amino)phenyl]sulfonyl}piperidin-4-yl)amino]ethanol (Compound CLVIII)

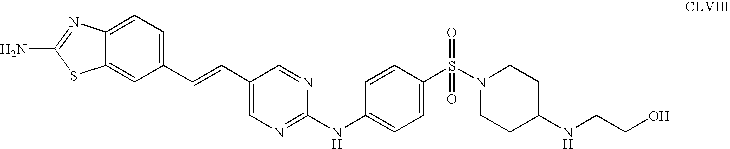

Solution of intermediate 183 (210.0 mg, 0.362 mmol) in 1 mL of DMSO was diluted with 6 mL of anhydrous THF and solid LiAlH₄ was added in 2.0 equiv portions every 3 h. The reaction progress was monitored by LC/MS. After 9 h and total of 6.0 equiv of LiAlH₄, the reaction was complete. The reaction mixture was quenched with 1 mL of TFA and concentrated in vacuo down to ca. 2 mL. 2 mL of DMF was added, the resulting mixture was filtered though 0.2 micron syringe filter and purified by reverse-phase preparative HPLC in CH₃CN/H₂O system containing 0.05% of TFA. All fractions containing the product were combined and poured into ca. 100 mL of EtOAc. The resulting solution was washed with sat. NaHCO₃ (2×100 mL), brine (2×100 mL), dried over anhydrous Na₂SO₄, filtered and concentrated in vacuo to give the title product as a pale-yellow solid (40.1 mg, 20%).

¹H NMR (500 MHz, DMSO-d₆): δ 1.23-1.29 (m, 2H), 1.79-1.82 (m, 2H), 2.36-2.42 (m, 3H), 3.38-3.42 (m, 2H), 7.07 (d, J=16.6 Hz, 1H), 7.32 (d, J=16.6 Hz, 1H), 7.33 (d, J=8.2 Hz, 1H), 7.44 (dd, J=8.4, 1.5 Hz, 1H), 7.57 (s, 2H), 7.65 (d, J=8.9 Hz, 2H), 7.88 (d, J=1.4 Hz, 1H), 8.02 (d, J=8.9 Hz, 2H), 8.80 (s, 2H), 10.30 (s, 1H). MS (ES+): m/z 552 (M+H)⁺.

### Example 344

### 1-tert-Butyl 3-methyl 4-[(4-bromophenyl)sulfonyl]piperazine-1,3-dicarboxylate (Intermediate 184)

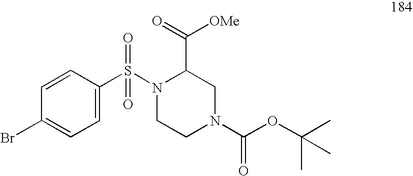

To a solution of 1-tert-butyl 3-methyl piperazine-1,3-dicarboxylate (1.0 g, 4.10 mmol) and Et₃N (0.456 g, 4.51 mmol) in 80 mL of DCM was added 4-bromobenzenesulfonyl chloride (1.05 g, 4.1 mmol). The reaction mixture was stirred at ambient temperature for 4 h. Then it was diluted with ca. 100 mL of EtOAc and washed with DI water (2×100 mL), 0.5 N HCl (1×50 mL), sat. NaHCO₃ (2×50 mL), brine (2×100 mL), dried over anhydrous Na₂SO₄, filtered though a short pad of silica gel and concentrated in vacuo to give the title intermediate as a white solid (1.87 g, 99%). MS (ES+): m/z 464 (M+H)⁺.

### Example 345

### tert-Butyl 4-[(4-bromophenyl)sulfonyl]-3-(hydroxymethyl)piperazine-1-carboxylate (Intermediate 185)

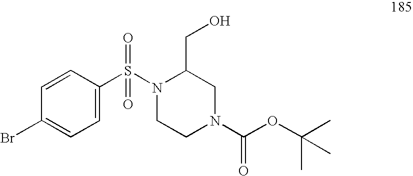

To a solution of intermediate 184 (1.87 g, 4.03 mmol) in 60 mL of anhydrous THF was added solid LiAlH₄ (95%, 306.3 mg, 8.07 mmol) in small portions. The reaction mixture was stirred at ambient temperature for 3 h. 10 mL of EtOAc was added to quench the reaction and the mixture was stirred for 30 min. Then it was poured into ca. 200 mL of H₂O and the resulting mixture was extracted with EtOAc (4×100 mL). Combined organic extracts were washed with brine (2×100 mL), dried over anhydrous Na₂SO₄, filtered though a short pad of silica gel and concentrated in vacuo to give the title intermediate as an off-white solid (1.43 g, 82%). MS (ES+): m/z 436 (M+H)⁺.

### Example 346

### tert-Butyl 3-(hydroxymethyl)-4-({4-[(5-vinylpyrimidin-2-yl)amino]phenyl}sulfonyl)piperazine-1-carboxylate (Intermediate 186)

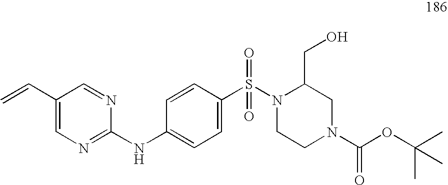

150 mL round-bottom flask was charged with the intermediate 1 (798.6 mg, 6.6 mmol), intermediate 185 (2.87 g, 6.6 mmol), Pd₂(dba)₃ (242 mg, 0.264 mmol), Xantphos (458.0 mg, 0.791 mmol), cesium carbonate (4.3 g, 13.18 mmol) and 100 mL of anhydrous dioxane. The reaction mixture was purged with the argon gas for 30 min; then it was heated to reflux and refluxed under argon atmosphere for 18 h. The completion of the reaction was monitored by TLC and LC/MS. Upon completion the reaction mixture was cooled down to ambient temperature, diluted with ca. 100 mL of EtOAc and filtered though a short pad of silica gel. The silica gel pad was washed with EtOAc. Combined organic solutions were concentrated in vacuo with ca. 15 g of silica gel. The loaded silica gel was taken to the ISCO system for further purification (80 g column, solid method, 0% to 100% EtOAc gradient in hexanes, 45 mm method, 310 nm detection wavelength). Fractions containing the product were combined and concentrated in vacuo to give a yellow solid. The solid was re-crystallized from ca. 30 mL of MeOH, filtered, washed with Et₂O and dried in vacuo to give the title intermediate as a cream-colored solid (1.4 g, 45%).

¹H NMR (500 MHz, DMSO-d₆): δ 1.33 (s, 9H), 2.63 (br s, 1H), 2.75 (dd, J=13.4, 4.0 Hz, 1H), 3.04 (br s, 1H), 3.29-3.35 (m, 1H), 3.35-3.39 (m, 1H), 3.55 (d, J=13.8 Hz, 1H), 3.75 (br s, 2H), 3.90 (br s, 1H), 4.83 (dd, J=6.1, 4.4 Hz, 1H), 5.29 (d, J=11.5 Hz, 1H), 5.92 (d, J=17.8 Hz, 1H), 6.66 (dd, J=17.8, 11.3 Hz, 1H), 7.75 (d, J=8.9 Hz, 2H), 7.99 (d, J=9.0 Hz, 2H), 8.72 (s, 2H), 10.31 (s, 1H). MS (ES+): m/z 476 (M+H)⁺.

### Example 347

### (1-{[4-({5-[(E)-2-(2-Amino-1,3-benzothiazol-6-yl)vinyl]pyrimidin-2-yl}amino)phenyl]sulfonyl}piperazin-2-yl)methanol (Compound CLIX)

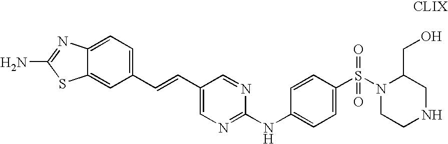

20 ml microwave vial was charged with intermediate 186 (475.6 mg, 1.0 mmol), 2-amino-6-bromobenzothiazole (275.0 mg, 1.2 mmol), Pd₂(dba)₃ (91.6 mg, 0.1 mmol), P(t-Bu)₃ (0.8 mL, 0.8 mmol, 1.0 M solution in toluene), cesium carbonate (651.6 g, 2.0 mmol), and anhydrous dioxane (18 mL). The mixture was purged with argon gas for 20 min, then sealed and irradiated in a microwave (Initiator, Biotage) at 180° C. for 1 h. After cooling to room temperature, the resulting mixture was diluted with ca. 100 mL of EtOAc and filtered though a short pad of silica gel. The silica gel pad was washed with 10% MeOH in EtOAc. Combined organic solutions were concentrated in vacuo and the residue was taken to the ISCO system for further purification (40 g column, solid method, 0% to 10% MeOH gradient in EtOAc, 45 min method, 350 nm detection wavelength). Fractions, containing the Boc-protected product, were combined and concentrated in vacuo to afford the Boc-protected product as a bright-yellow solid (150 mg, 24%). The solid was treated with 50% TFA in DCM for 10 min. The resulting solution was concentrated in vacuo and the residue was purified by reverse-phase preparative HPLC in CH₃CN/H₂O system containing 0.05% of TFA. Fractions containing the product were combined and poured into ca. 80 mL of saturated aqueous NaHCO₃. The resulting precipitate was collected by centrifugation, washed with water (2×40 mL), MeOH (1×5 mL), Et₂O (2×40 mL) and dried in vacuo to give the title compound as a yellow solid (70.0 mg, 13%).

¹H NMR (500 MHz, DMSO-d₆): δ 2.27 (dt, J=12.2, 3.3 Hz, 1H), 2.35 (dd, J=12.4, 3.7 Hz, 1H), 2.66 (d, J=11.8 Hz, 1H), 2.92 (d, J=12.0 Hz, 1H), 2.97 (dd, J=12.8, 2.7 Hz, 1H), 3.17 (d, J=2.6 Hz, 1H), 3.45 (d, J=12.9 Hz, 1H), 3.60-3.62 (m, 1H), 3.73 (t, J=9.7 Hz, 1H), 4.73 (br s, 1H), 7.07 (d, J=16.6 Hz, 1H), 7.31 (d, J=16.6 Hz, 1H), 7.33 (d, J=8.3 Hz, 1H), 7.44 (dd, J=8.4, 1.5 Hz, 1H), 7.57 (s, 2H), 7.72 (d, J=8.9 Hz, 2H), 7.88 (d, J=1.3 Hz, 1H), 7.99 (d, J=9.0 Hz, 2H), 8.80 (s, 2H), 10.28 (s, 1H). MS (ES+): m/z 524 (M+H)⁺.

### Example 348

### 5-(3-(Difluoromethyl)styryl)-N-(4-(piperidin-4-ylsulfonyl)phenyl)pyrimidin-2-amine (Compound CLX)

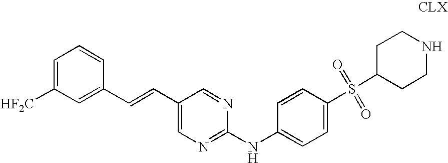

A mixture of 3-bromobenzaldehyde (585 μl, 5 mmol) and deoxofluor (3.31 g, 15 mmol) in dichloromethane (60 mL) was heated under reflux for 2 h. The reaction mixture was quenched with aqueous saturated NaHCO₃ (30 mL) and extracted with dichloromethane (2×50 mL). The organic layer was separated, dried (Na₂SO₄), and filtered through a small silica plug. On evaporation of the solvent, 1-bromo-3-(difluoromethyl)benzene was obtained as a brown syrup (1.0 g, quantitative). A mixture of precursor 1-bromo-3-(difluoromethyl)benzene (30 mg, 0.15 mmol), 6 (44 mg, 0.10 mmol), Pd(OAc)₂ (0.89 mg, 0.033 mmol), triphenyl phosphine (4.2 mg, 0.016 mmol) and KHCO₃ (40 mg, 0.4 mmol) in DMF (2 mL) was sealed in a microwave reaction vial and irradiated with microwave at 180° C. for 30 min. The reaction mixture was cooled to room temperature and purified by HPLC, and treated with TFA (1 mL) in dichloromethane (10 mL) for 10 minutes. The solvent was evaporated and the residue purified by HPLC to give the title compound as white solid (13 mg, 18%).

¹H NMR (500 MHz, DMSO-d₆): δ 1.62-1.72 (m, 2H), 2.01-2.07 (m, 2H), 2.45-2.60 (m, 2H overlapped with DMSO), 2.82-2.92 (m, 2H), 3.36-3.38 (m, 2H, overlapped with H₂O), 3.43-3.53 (m, 1H), 7.07 (t, J=56.0 Hz, 1H), 7.28 (d, J=16.5 Hz, 1H), 7.43 (d, J=16.5 Hz, 1H), 7.48 (d, J=7.8 Hz, 1H), 7.55 (t, J=8.0 Hz, 1H), 7.72-7.80 (m, 4H), 8.08 (d, J=8.9 Hz, 2H), 8.88 (s, 2H), 10.48 (s, 1H). MS (ES+): m/z 471 (M+H)⁺.

### Example 349

### 5-(4-(Difluoromethyl)styryl)-N-(4-(piperidin-4-ylsulfonyl)phenyl)pyrimidin-2-amine (Compound CLXI)

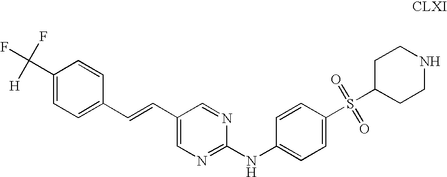

A mixture of 1-bromo-4-(difluoromethyl)benzene (30 mg, 0.15 mmol), intermediate 6 (44 mg, 0.10 mmol), Pd(OAc)₂ (0.89 mg, 0.033 mmol), triphenyl phosphine (4.2 mg, 0.016 mmol) and KHCO₃ (40 mg, 0.4 mmol) in DMF (2 mL) was sealed in a microwave reaction vial and irradiated with microwave at 180° C. for 30 min. The reaction mixture was cooled to room temperature and purified by HPLC, and treated with TFA (1 mL) in dichloromethane (10 mL) for 10 minutes. The solvent was evaporated and the residue purified by HPLC to give the title compound as cream colored solid (13 mg, 18%).

¹H NMR (500 MHz, DMSO-d₆): δ 1.62-1.72 (m, 2H), 1.97-2.07 (m, 2H), 2.45-2.60 (m, 2H overlapped with DMSO), 2.82-2.95 (m, 2H), 3.36-3.38 (m, 2H, overlapped with H₂O), 3.43-3.53 (m, 1H), 7.04 (t, J=56.0 Hz, 1H), 7.30 (d, J=16.5 Hz, 1H), 7.41 (d, J=16.5 Hz, 1H), 7.59 (d, J=8.4 Hz, 2H), 7.71 (d, J=8.3 Hz, 2H), 7.76 (d, J=9.0 Hz, 2H), 8.08 (d, J=9.1 Hz, 2H), 8.88 (s, 2H), 10.49 (s, 1H). MS (ES+): m/z 471 (M+H)⁺.

### Example 350

### 5-(3-(Trifluoromethyl)styryl)-N-(4-(piperidin-4-ylsulfonyl)phenyl)pyrimidin-2-amine (Compound CLXII)

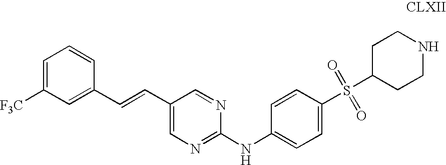

A mixture of 3-bromo-benzotrifluoride (33 mg, 0.15 mmol), intermediate 6 (66 mg, 0.15 mmol), Pd(OAc)₂ (0.89 mg, 0.033 mmol), triphenyl phosphine (4.2 mg, 0.016 mmol) and KHCO₃ (40 mg, 0.4 mmol) in DMF (2 mL) was sealed in a microwave reaction vial and irradiated with microwave at 180° C. for 30 min. The reaction mixture was cooled to room temperature and purified by HPLC, and treated with TFA (1 mL) in dichloromethane (10 mL) for 10 minutes. The solvent was evaporated and the residue purified by HPLC to give the title compound as white solid (11 mg, 15%).

¹H NMR (500 MHz, DMSO-d₆): δ 1.60-1.72 (m, 2H), 1.95-2.07 (m, 2H), 2.45-2.60 (m, 2H overlapped with DMSO), 2.82-2.92 (m, 2H), 3.36-3.38 (m, 2H, overlapped with H₂O), 3.43-3.53 (m, 1H), 7.37 (d, J=16.7 Hz, 1H), 7.46 (d, J=16.9 Hz, 1H), 7.62-7.66 (m, 2H), 7.76 (d, J=9.0 Hz, 2H), 7.85-7.92 (m, 1H), 7.92 (s, 1H), 8.08 (d, J=9.3 Hz, 2H), 8.87 (s, 2H), 10.50 (s, 1H). MS (ES+): m/z 489 (M+H)⁺.

### Example 351

### 5-(3-(Methyl)styryl)-N-(4-(piperidin-4-ylsulfonyl)phenyl)pyrimidin-2-amine (Compound CLXIII)

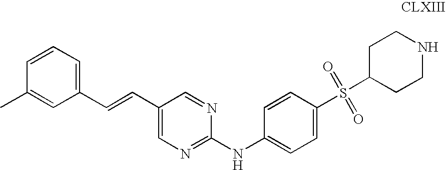

A mixture of 1-bromo-3-methylbenzene (26 mg, 0.15 mmol), intermediate 6 (66 mg, 0.15 mmol), Pd(OAc)₂ (0.89 mg, 0.033 mmol), triphenyl phosphine (4.2 mg, 0.016 mmol) and KHCO₃ (40 mg, 0.4 mmol) in DMF (2 mL) was sealed in a microwave reaction vial and irradiated with microwave at 180° C. for 30 min. The reaction mixture was cooled to room temperature and purified by HPLC, and treated with TFA (1 mL) in dichloromethane (10 mL) for 10 minutes. The solvent was evaporated and the residue purified by HPLC to give the title compound as white solid (13 mg, 20%).

¹H NMR (500 MHz, DMSO-d₆): δ 1.60-1.72 (m, 2H), 1.95-2.07 (m, 2H), 2.33 (s, 3H), 2.45-2.60 (m, 2H overlapped with DMSO), 2.82-2.92 (m, 2H), 3.36-3.38 (m, 2H, overlapped with H₂O), 3.43-3.53 (m, 1H), 7.11 (d, J=7.4 Hz, 2H), 7.16 (d, J=17.0 Hz, 1H), 7.25-7.32 (m, 1H), 7.27 (d, J=16.4 Hz, 1H), 7.36-7.44 (m, 2H), 7.76 (d, J=8.9 Hz, 2H), 8.08 (d, J=8.9 Hz, 2H), 8.15-8.25 and 8.55-8.65 (2 br s, 1H each), 8.85 (s, 2H), 10.45 (s, 1H). MS (ES+): m/z 436 (M+H)⁺.

### Example 352

### 3-((E)-2-(2-(4-(Piperidin-4-ylsulfonyl)phenylamino)pyrimidin-5-yl)vinyl)-5-(trifluoro methyl)benzene-1,2-diamine (Compound CLXIV)

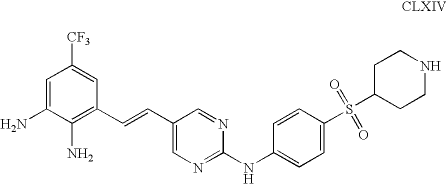

A mixture of 3-bromo-4,5-diamino-benzotrifluoride (255 mg, 1.0 mmol), intermediate 6 (444 mg, 1.0 mmol), Pd(OAc)₂ (4.5 mg, 0.02 mmol), triphenyl phosphine (21 mg, 0.08 mmol) and KHCO₃ (200 mg, 2.0 mmol) in DMF (5 mL) was sealed in a microwave reaction vial and irradiated with microwave at 180° C. for 30 min. The reaction mixture was purified by HPLC to give the title compound as white solid (183 mg, 30%).

¹H NMR (500 MHz, DMSO-d₆): δ 1.60-1.72 (m, 2H), 1.95-2.07 (m, 2H), 2.82-2.95 (m, 2H), 2.82-2.92 (m, 2H), 3.33-3.40 (m, 2H), 3.43-3.53 (m, 1H), 6.79 (s, 1H), 7.01 (d, J=16.3 Hz, 1H), 7.15 (s, 1H), 7.52 (d, J=17.5 Hz, 1H), 7.75 (d, J=8.9 Hz, 2H), 8.08 (d, J=9.0 Hz, 2H), 8.15-8.25 and 8.55-8.63 (2 br s, 1H each), 8.91 (s, 2H), 10.43 (s, 1H). MS (ES+): m/z 519 (M+H)⁺.

### Example 353

### 5-[2-(3-Methoxy-phenyl)-vinyl]-pyrimidin-2-ylamine (Intermediate 187)

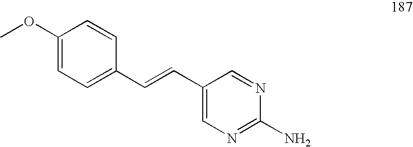

In a dry 25 mL round bottom flask were combined trans-2-(4-methoxyphenyl)vinyl boronic acid (0.494 g, 2.8 mmol), 5-bromo-pyrimidin-2-ylamine (0.4 g, 2.3 mmol), Pd(PPh₃)₄ (0.27 g) and sodium carbonate (0.981 g). These were then diluted with a mixture of DME (12 mL), EtOH (2 mL) and water (2 mL) and heated to reflux. After 7 h, reaction was filtered hot and solvents removed. The resulting residue was diluted with ethyl acetate (100 mL) and washed with water (1×200 mL) and brine (1×100 mL). Organic phase dried over sodium sulfate, filtered and adsorbed onto silica gel. Crude product was purified on ISCO normal phase column (80 gram). A 10% ethyl acetate in hexanes gradient ramping up to 100% ethyl acetate provided a pure product. Yellow powder (0.35 g, 67%).

### Example 354

### 4-(4-{5-[2-(3-Methoxy-phenyl)-vinyl]-pyrimidin-2-ylamino}-benzenesulfonyl)-piperidine-1-carboxylic acid tert-butyl ester (Intermediate 188)

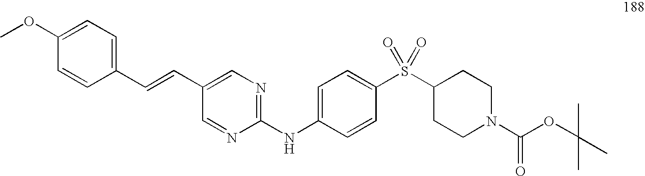

In a dry 15 mL microwave vial were combined intermediate 187, (0.073 g, 0.32 mmol), 5 (0.16 g, 0.39 mmol), cesium carbonate (0.31 g, 0.96 mmol), xantphos (0.037 g, 0.064 mmol) and Pd₂(dba)₃ (0.029 g, 0.032 mmol, 0.1 equiv) were combined. The reactants were flushed with argon, diluted with dioxane (6 mL) and microwaved for 15 min at 160° C. Reaction was then spun down, decanted and solvents removed. The residue then diluted with DCM and adsorbed onto silica gel. Crude product was purified on ISCO normal phase column (80 gram). A 10% ethyl acetate in Hexanes gradient ramping up to 75% ethyl acetate in Hexanes provided pure product. Yellow solids (0.18 g, 100%).

### Example 355

### 2-(2-{2-[4-(Piperidine-4-sulfonyl)-phenylamino]-pyrimidin-5-yl}-vinyl)-phenol (Compound CLXV)

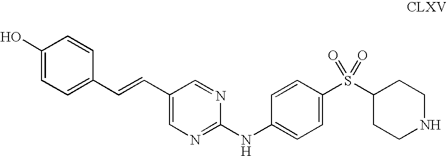

Intermediate 188, (0.12 g, 0.26 mmol) was diluted with 10 mL DCM and chilled to 0° C. using an ice bath. A 1.0 M solution of BBr₃ in DCM (2 mL, 2 mmol) was then added in several portions resulting in dark reaction mixture. Once addition was complete, reaction was allowed to come to ambient temperature and stir for 1 h. Reaction was then quenched by carefully adding MeOH (5 mL). Reaction solvents then removed and HPLC purification provided the TFA salt of desired product. Yellow solid (0.043 g, 38%).

¹H NMR (DMSO-d₆): δ 1.62-1.70 (m, 2H), 2.01-2.04 (d, 2H), 2.86 (br s, 2H), 3.42-3.49 (m, 1H), 6.70 (d, J=8.5 Hz, 2H), 6.94 (d, J=16.6 Hz, 1H), 7.24 (d, J=16.6 Hz, 1H), 7.4 (d, J=8.6 Hz, 2H), 7.74 (d, J=9.0 Hz, 1H), 8.06 (d, J=8.9 Hz, 2H), 8.24 (br s, 1H), 8.67 (br s, 1H), 8.79 (s, 2H), 9.65 (s, 1H), 10.4 (s, 1H). MS (ES+): m/z 437 (M+H)⁺.

### Example 356

### Determination of Abl and Abl (T3151) Kinase Activity in Enzyme Assays

Kinase activity was assessed using luminescent detection (KinaseGlo, Promega) of residual ATP concentration in reaction mixtures containing optimized levels of kinase, substrate, ATP, compound and appropriate buffers. Kinase, substrate and ATP concentrations were determined per the supplier's recommendations. Briefly, this involved running concentration curves to select for optimal signal.

Abl Kinase Assay

The reaction mixture (50 ul/well) consisted of Abl kinase (Invitrogen, 20 ng/well); Abltide peptide substrate (Upstate, 100 μM) and ATP (500 nM). Each compound was evaluated at a top concentration of 10 uM with 1:3 dilution steps, 10 dilution steps total. Compound was diluted into DMSO and transferred to the reaction plate, resulting in a final DMSO concentration of 2%. This amount of DMSO was determined not to interfere with the enzyme. The reaction was conducted at 37° C. for 60 minutes. The degree of inhibition was assessed using IC50 determinations, which were obtained using GraphPad Prism4.

Abl (T315I) Kinase Assay

The assay was performed similarly to the Abl kinase assay. Abl (T315I) (Upstate, 17 ng/well); Abltide substrate (100 uM) and ATP (500 nM) comprised the reaction mixture. Compound was similarly diluted, and the reaction plate was incubated for 60 minutes at 37° C.

### Example 357

### Cell Based Assays

Construction of Ba/F3:BCR/ABL Cell Lines:

The human BCR cDNA containing the 3 N-terminal exons and the human ABL cDNA minus the first exon were fused in frame enzymatically and then inserted into a retroviral plasmid vector that carries a neomycin resistant gene (pFB.Neo). The recombinant plasmid was introduced into a retrovirus packaging cell line (EcoPack2-293) through calcium phosphate-mediated transfection to produce replication deficient retrovirus that expresses the BCR/ABL fusion protein. Following the collection of the recombinant retrovirus-containing medium in the transfected EcoPack2-293 cells, a mouse pro-B cell line, BaF3, was infected with the recombinant retrovirus. The Ba/F3 cells that have up-taken the recombinant retrovirus and permanently incorporated the viral DNA into the genome were selected by adding G418 to the culture medium at a final concentration of 1 mg/ml. The expression of ˜300 kD BCR/ABL fusion protein was confirmed by Western blot via the presence of the BCR/ABL fusion protein and stimulated phosphorylation of BCR/ABL substrates such as ABL, CrkL and STAT5. To introduce point mutations into the ABL kinase domain of the BCR/ABL fusion protein, site-directed mutagenesis was performed on the recombinant retroviral plasmid vector following the insertion of BCR/ABL fusion cDNA. The introduced point mutations were subsequently confirmed by DNA sequencing prior to transfection in EcoPack2-293 cells.

### Example 358

### Cell Proliferation Assays

Compounds were evaluated for their ability to inhibit the proliferation of Ba/F3 cells over-expressing the following mutant forms of BCR-ABL: no point mutation; T315I, F317L and M351T. Potentially toxic effects were assessed using the parental Ba/F3 cell line.

Cells were plated at 2500 cells/well. Compounds were pre-diluted at 200× in cell culture medium (10% FBS, Penicillin/Streptomycin/Glutamine in RPMI, plus 10% IL3 for parental cells). The final concentration of DMSO, 0.5%, was determined to not be detrimental to cells. The compound concentration curve was 10 μM top, 1:3 dilution steps, 10 steps. Compound was added immediately after plating the cells, and cells were incubated for 72 hours at 37° C. Cell viability was assessed using XTT (Sigma, 1 mg/ml). IC₅₀ values were determined and normalized as described for the biochemical assays.

The results are of the enzymatic assays are shown in Table 1

TABLE 1
Structure

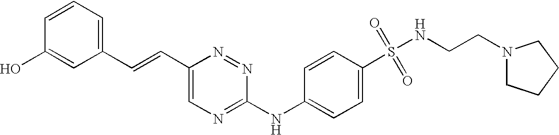

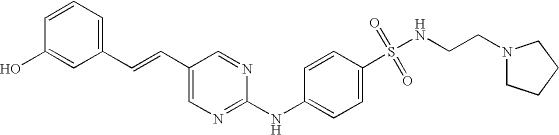

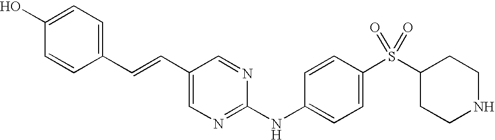

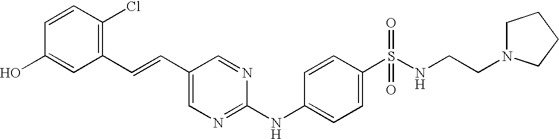

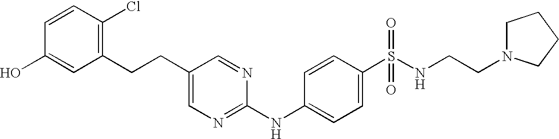

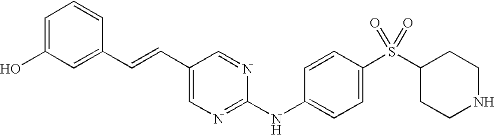

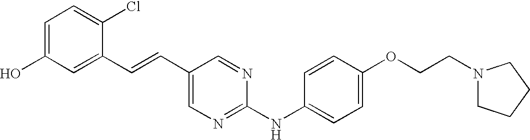

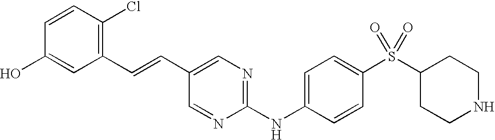

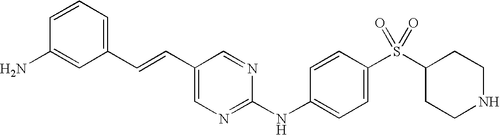

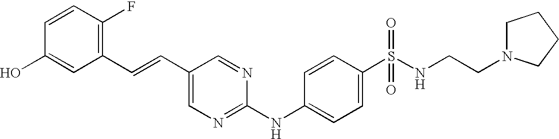

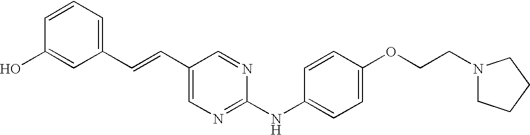

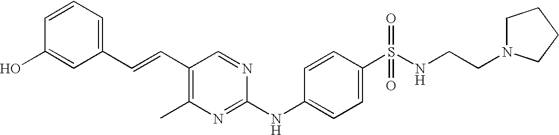

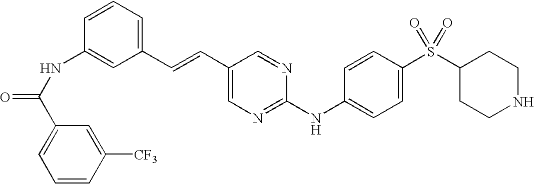

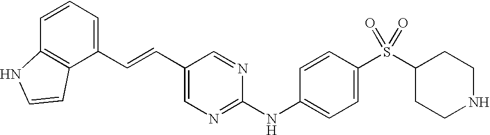

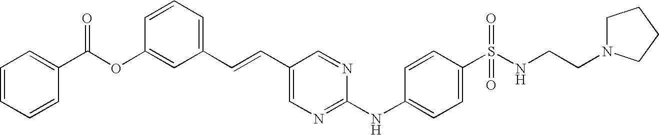

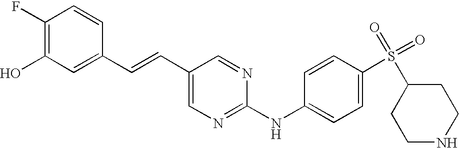

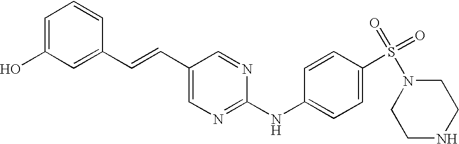

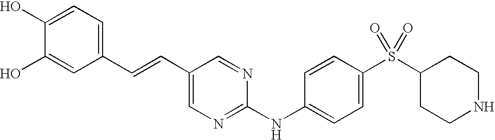

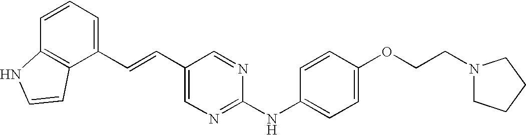

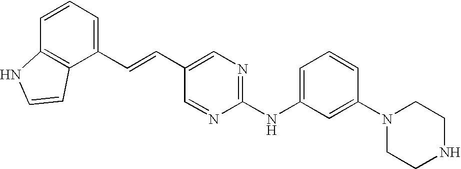

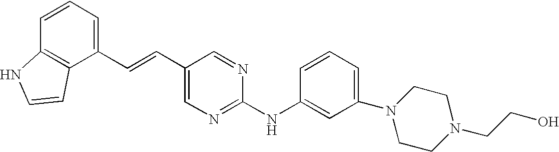

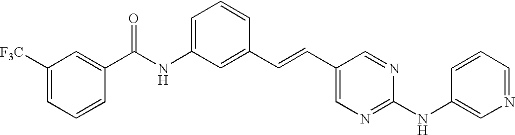

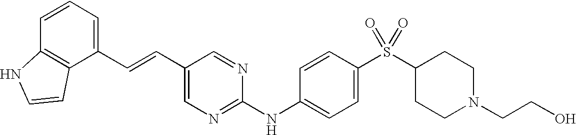

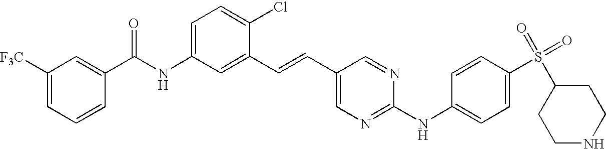

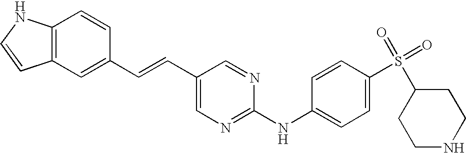

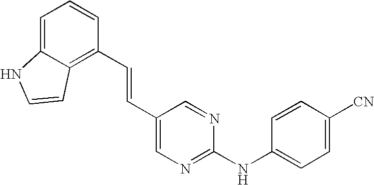

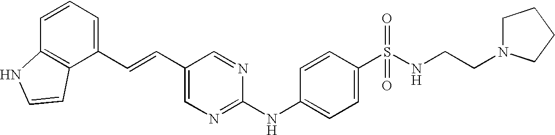

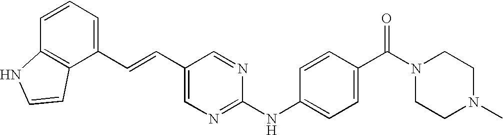

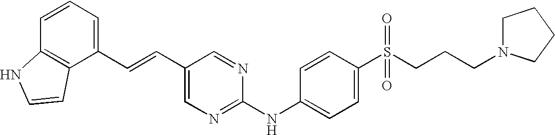

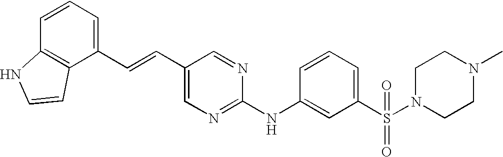

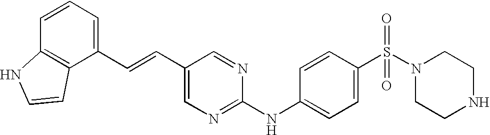

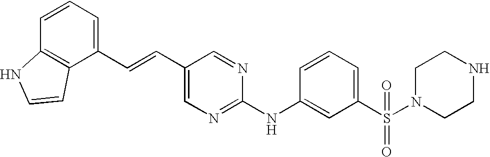

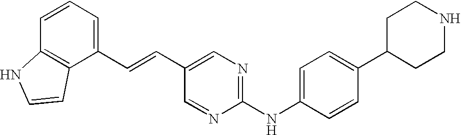

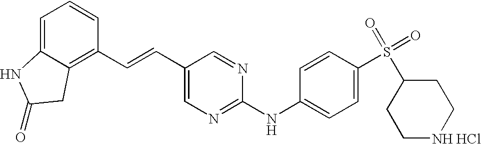

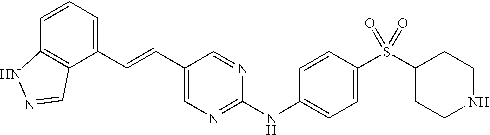

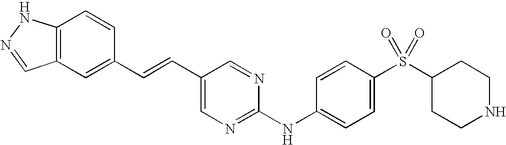

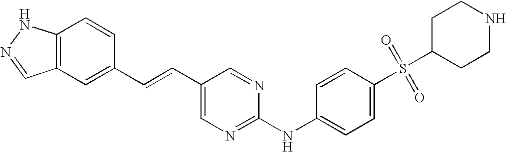

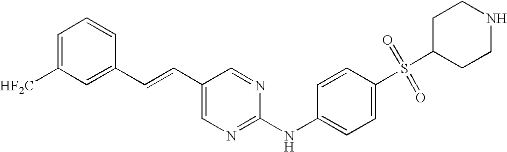

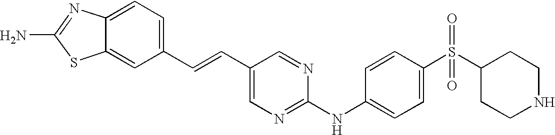

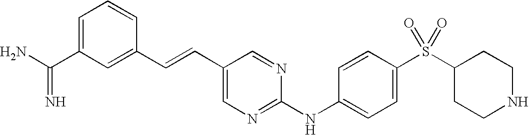

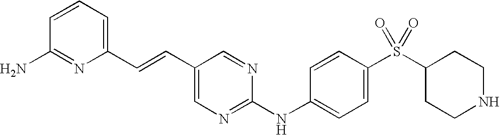

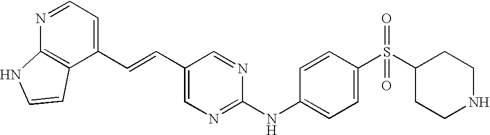

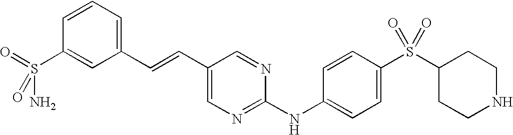

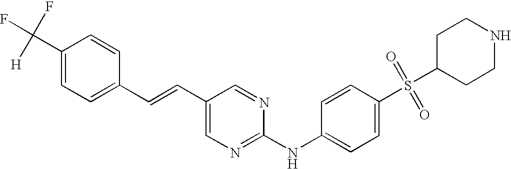

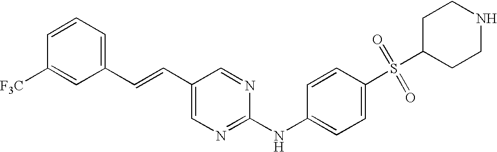

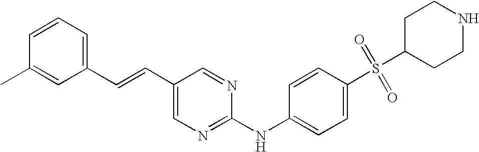

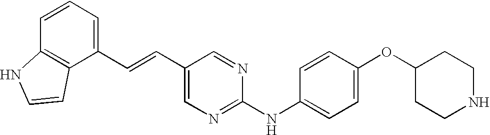

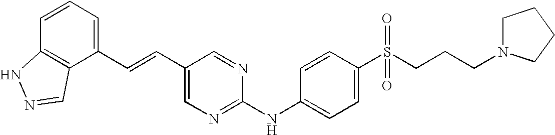

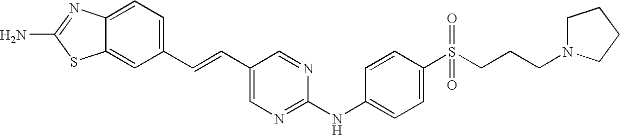

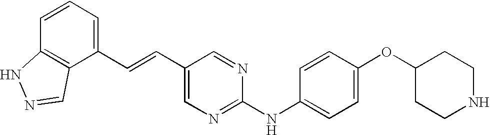

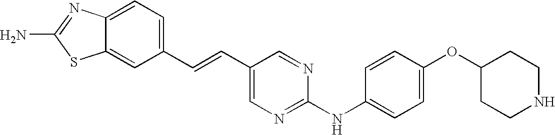

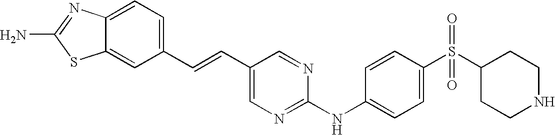

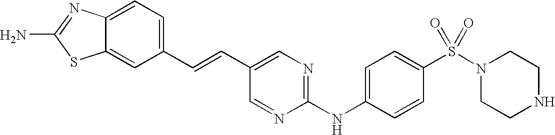

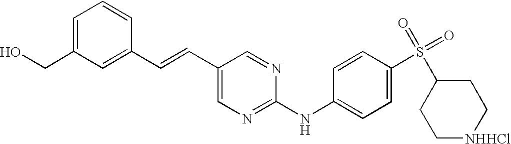

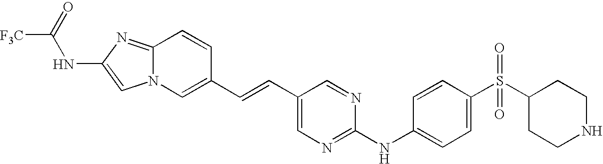

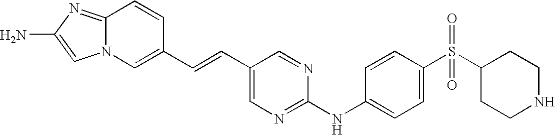

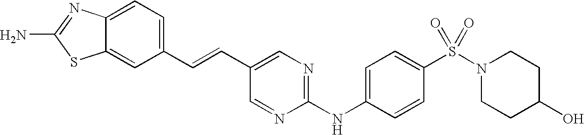

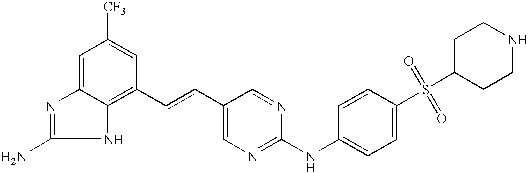

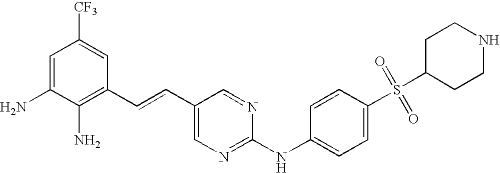

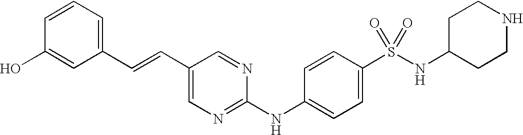

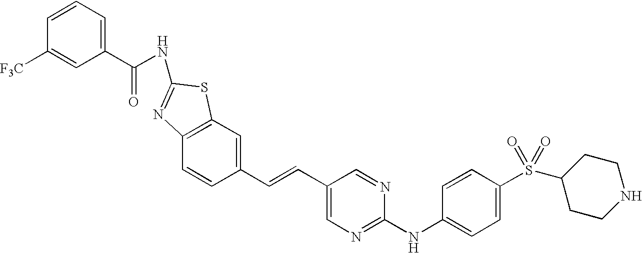

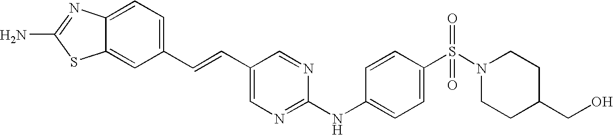

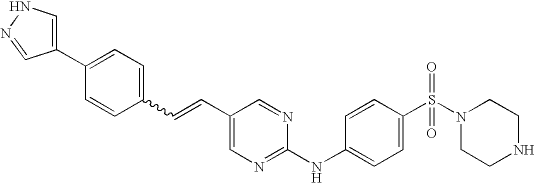

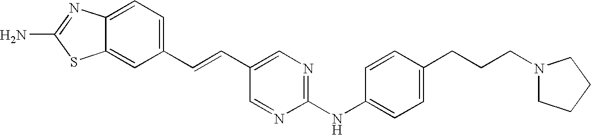

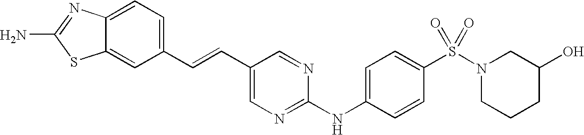

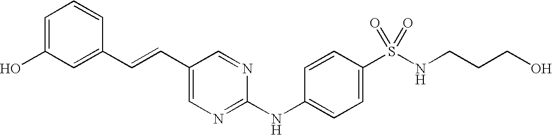

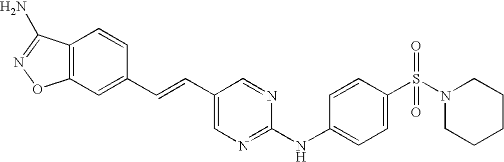

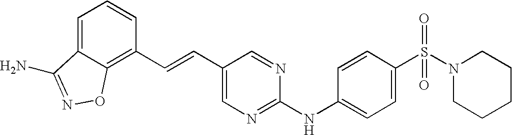

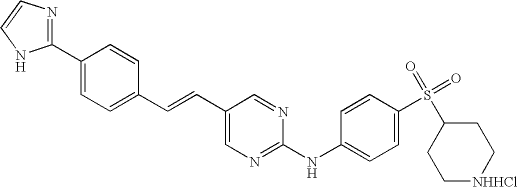

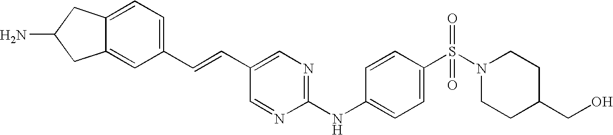

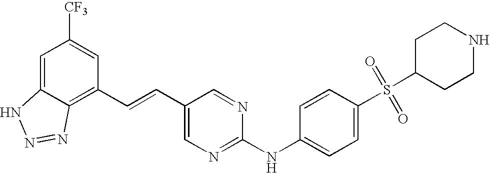

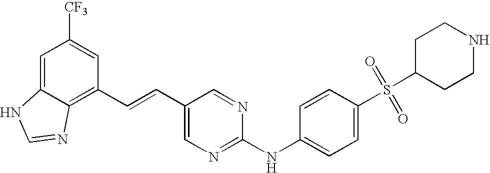

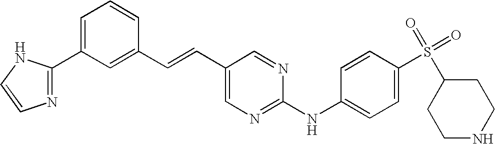

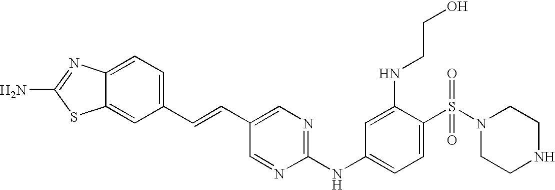

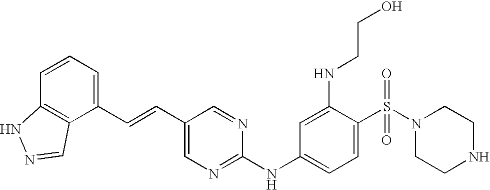

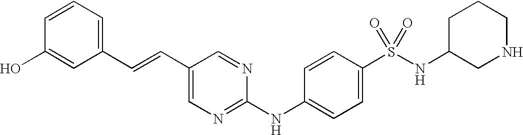

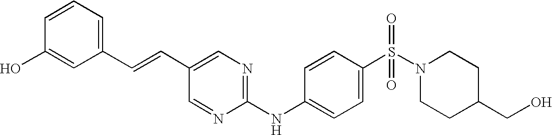

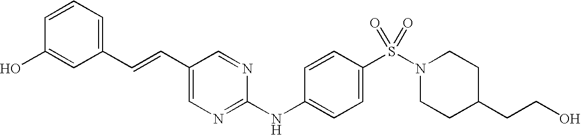

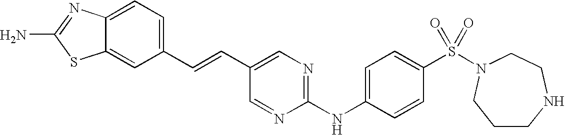

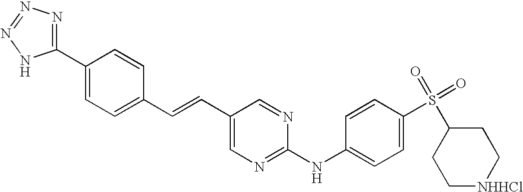

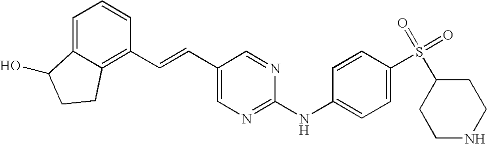

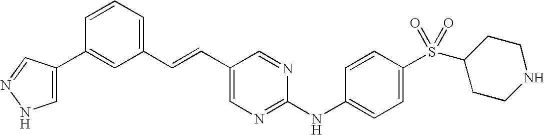

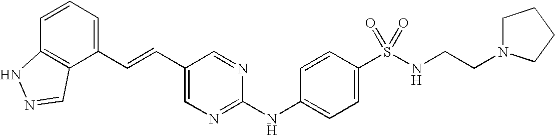

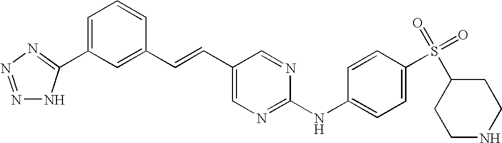

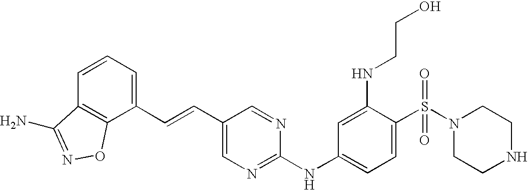

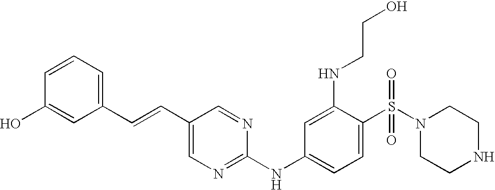

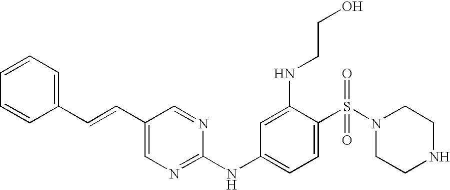

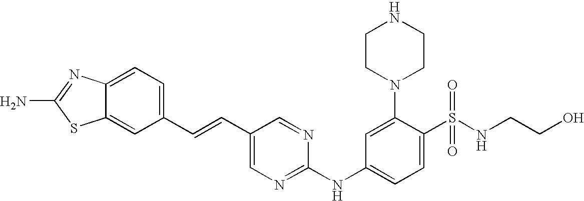

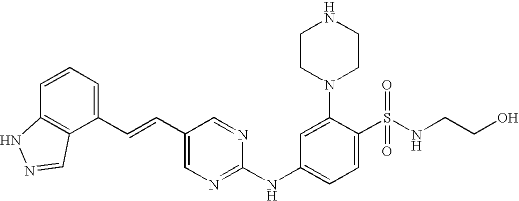

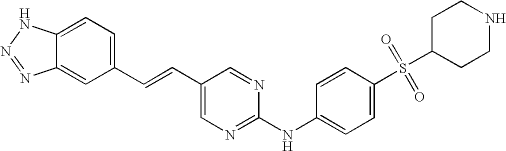

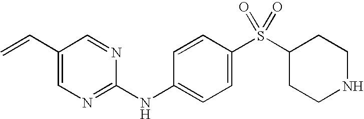

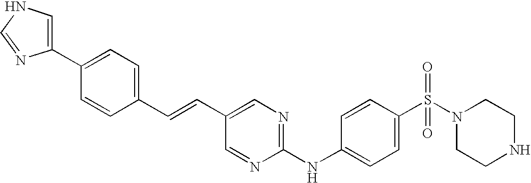

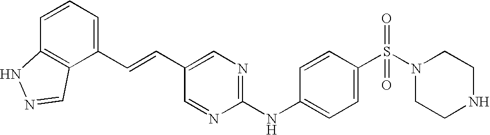

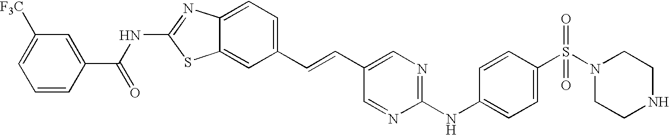

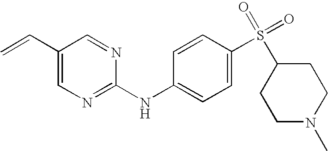

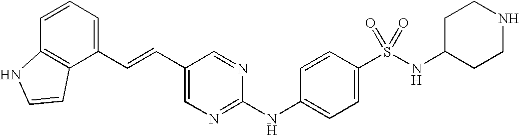

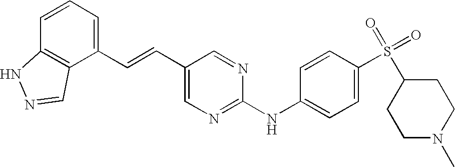

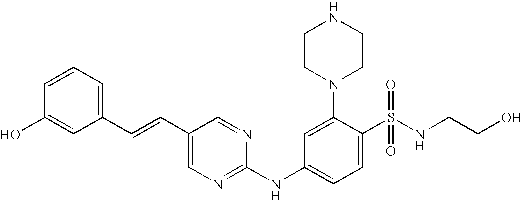

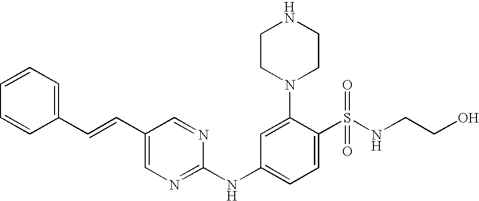

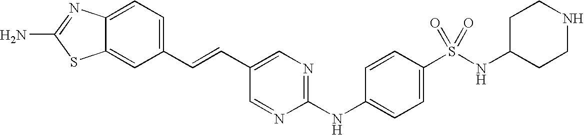

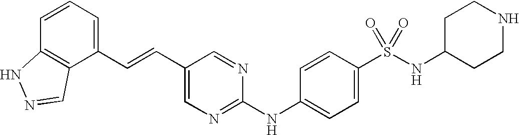

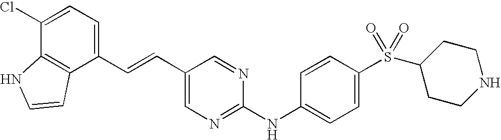

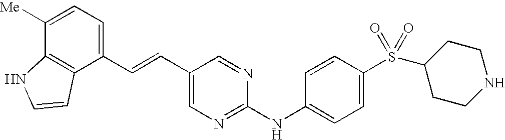

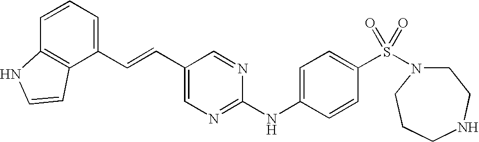

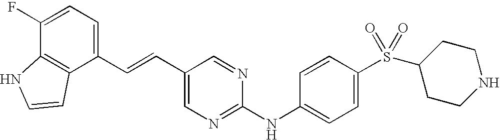

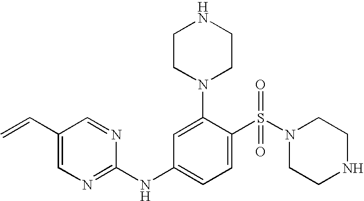

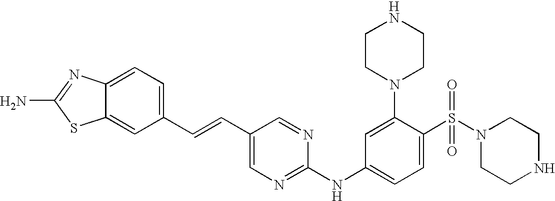

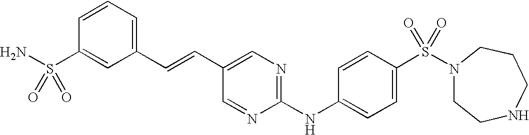

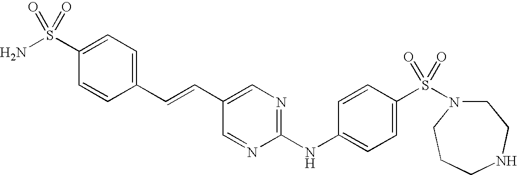

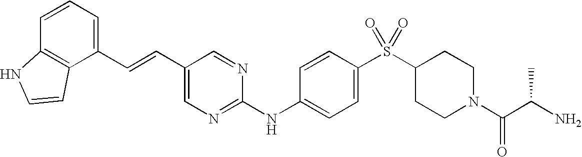

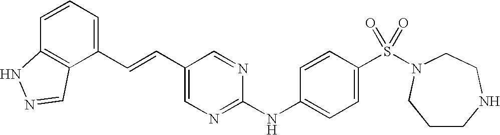

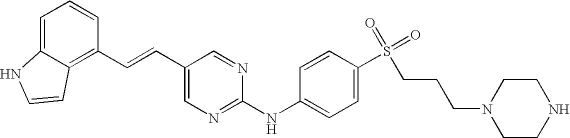

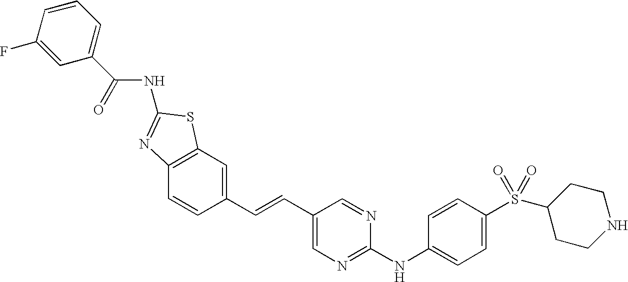

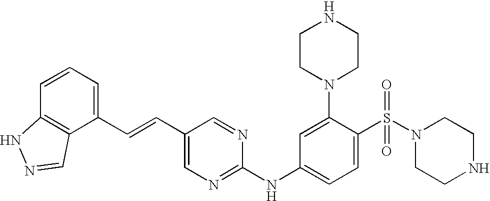

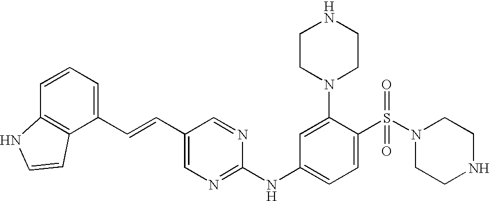

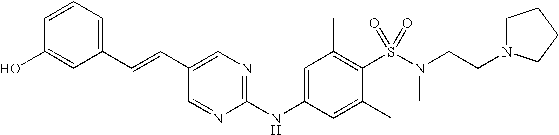

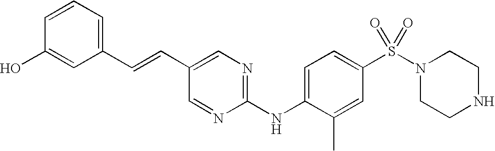

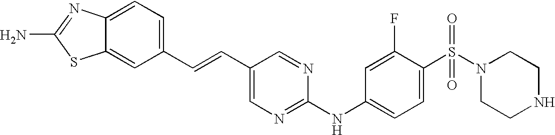

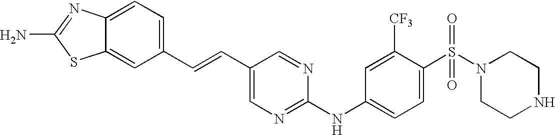

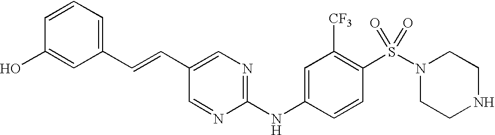

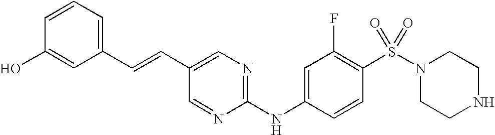

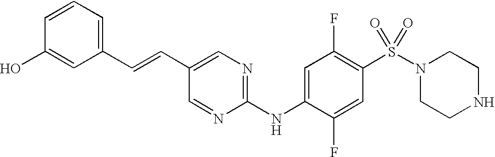

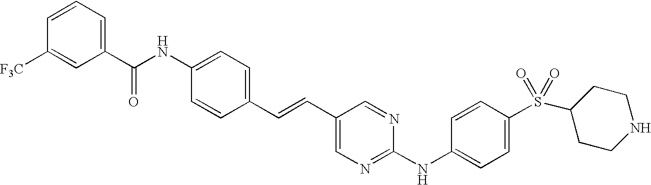

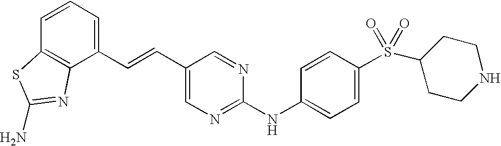

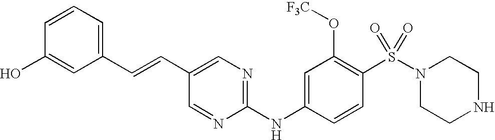

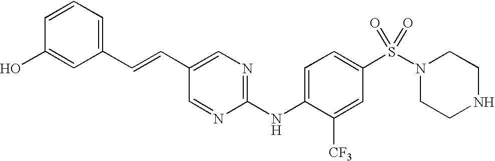

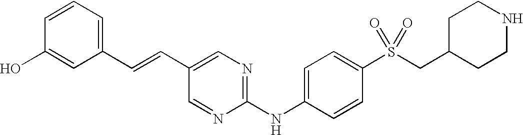

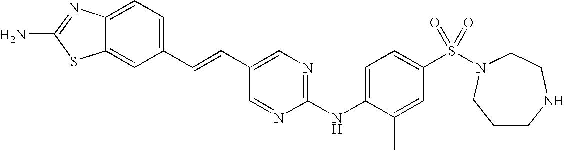

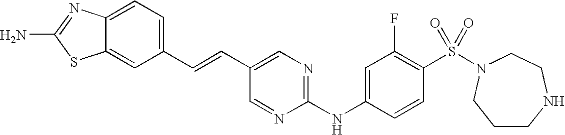

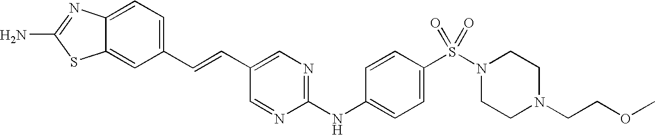

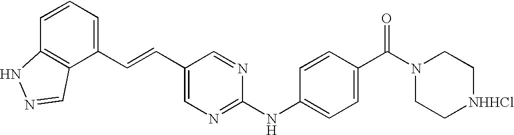

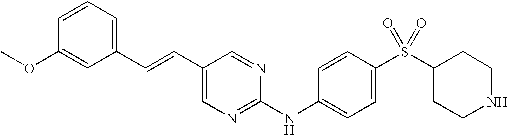

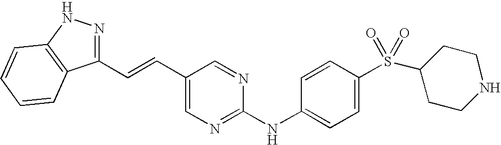

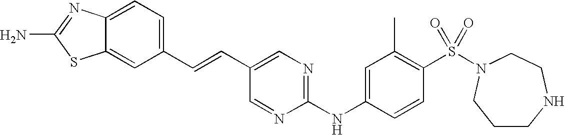

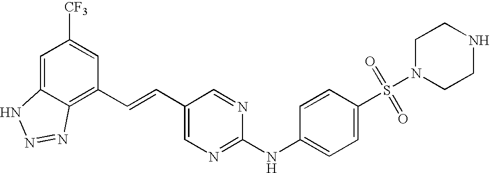

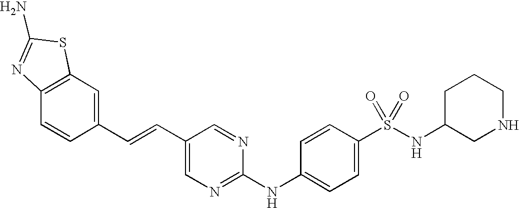

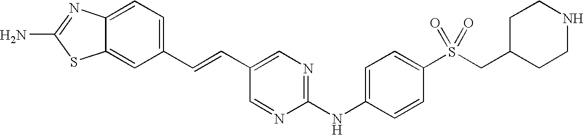

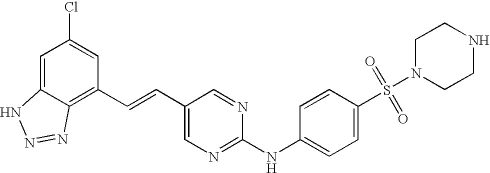

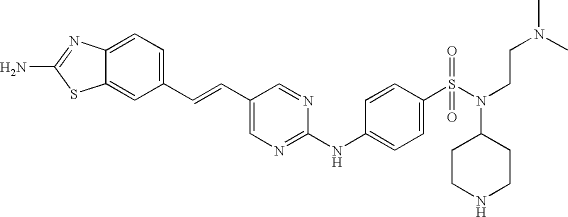

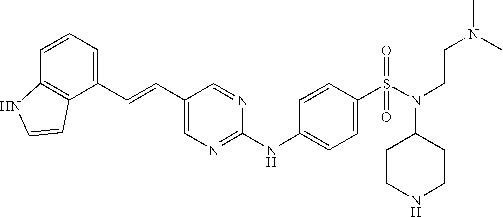

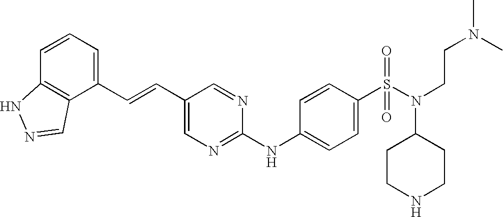

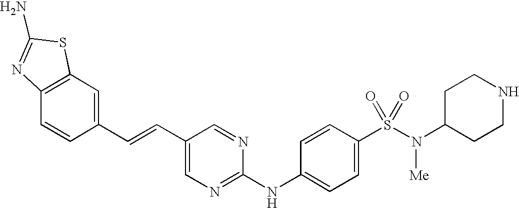

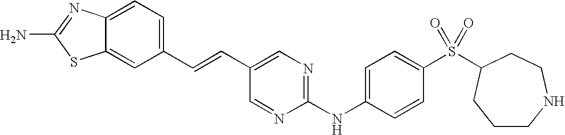

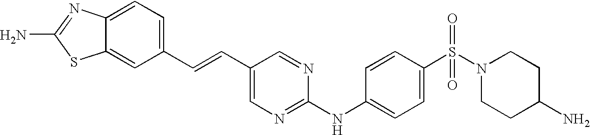

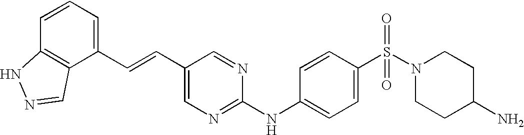

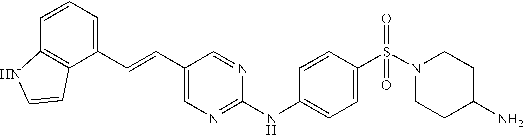

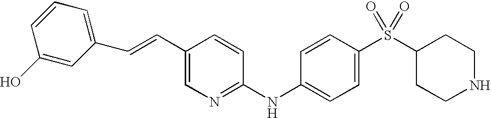

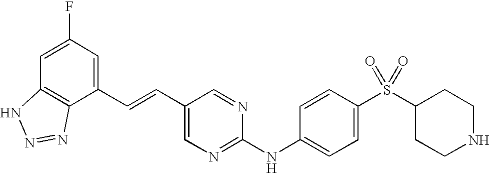

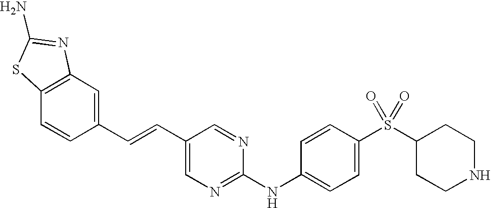

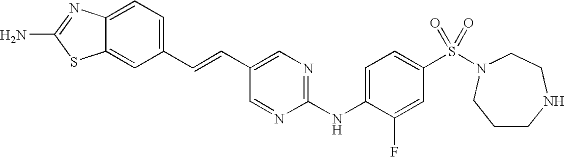

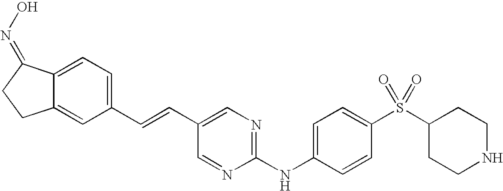

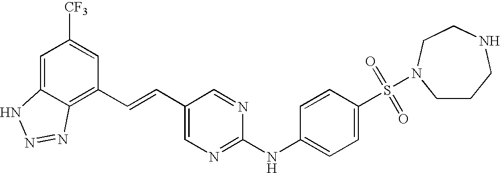

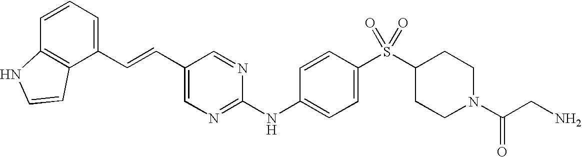

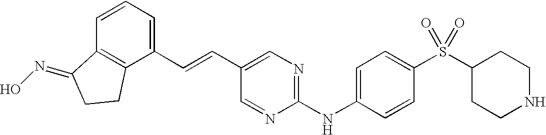

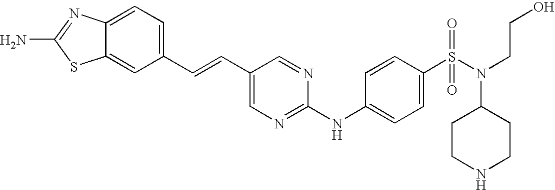

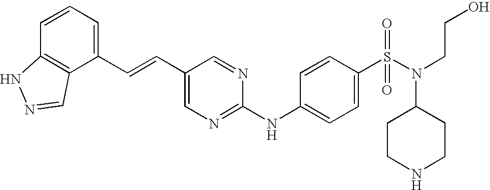

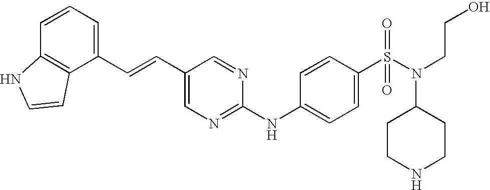

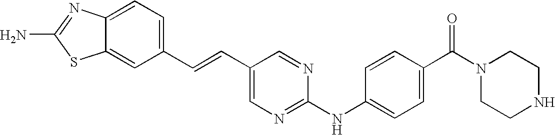

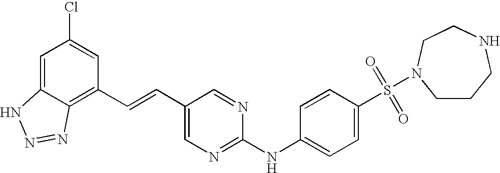

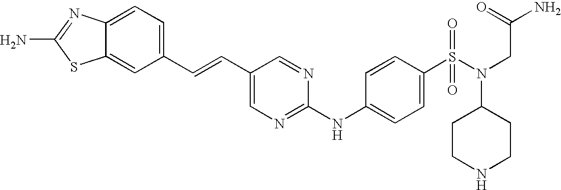

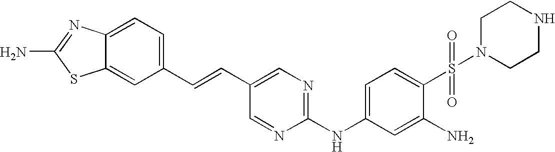

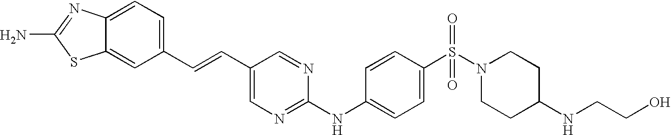

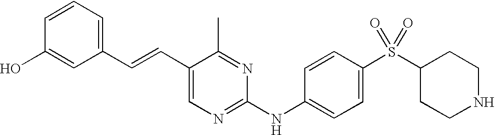

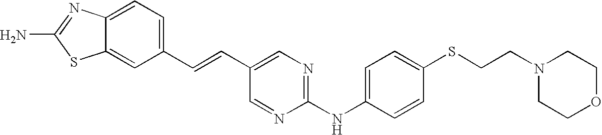

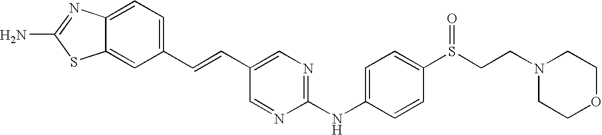

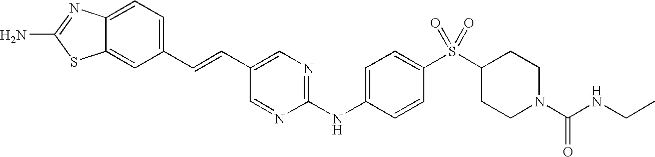

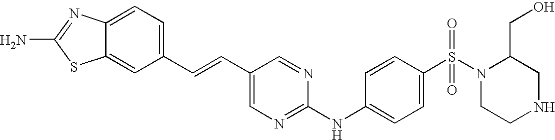

| | Abl | |
|---|---|---|
| | IC50 | Abl (T315I) |
| Name | (nM) | IC50 (nM) |
| 4-{6-[2-(3-Hydroxy-phenyl)- | N/A | N/A |
| vinyl]-[1,2,4]triazin-3- | | |
| ylamino}-N-(2-pyrrolidin-1- | | |
| yl-ethyl) | | |
| benzenesulfonamide | | |
| 4-{5-[2-(3-Hydroxy-phenyl)- | 7.1 | 141 |
| vinyl]-pyrimidin-2- | | |
| ylamino}-N-(2-pyrrolidin-1- | | |
| yl-ethyl)- | | |
| benzenesulfonamide | | |
| 4-(2-{2-[4-(Piperidine-4- | 15 | 32 |
| sulfonyl)-phenylamino]- | | |
| pyrimidin-5-yl}-vinyl)- | | |
| phenol | | |
| 4-{5-[2-(2-Chloro-5- | 7.7 | 390 |
| hydroxy-phenyl)-vinyl]- | | |
| pyrimidin-2-ylamino}-N-(2- | | |
| pyrrolidin-1-yl-ethyl)- | | |
| benzenesulfonamide | | |
| 4-{5-[2-(2-Chloro-5- | 5 | 805 |
| hydroxy-phenyl)-ethyl]- | | |
| pyrimidin-2-ylamino}-N-(2- | | |
| benzenesulfonamide | | |
| hydrochloride | | |
| 3-(2-{2-[4-(Piperidine-4- | 1.7 | 22 |
| sulfonyl)-phenylamino]- | | |
| pyrimidin-5-yl}-vinyl)- | | |
| phenol | | |
| 3-((E)-2-(2-(4-(2-(pyrrolidin- | 13 | 972 |
| 1- | | |
| yl)ethoxy)phenylamino)pyri- | | |
| midin-5-yl)vinyl)-4- | | |
| chlorophenol hydrochloride | | |
| 4-Chloro-3-(2-{2-[4- | 3.8 | 96 |
| (piperidine-4-sulfonyl)- | | |
| phenylamino]-pyrimidin-5- | | |
| yl}-vinyl)-phenol | | |
| 15-(3-aminostyryl)-N-(4- | 31 | 561 |
| (piperidin-4- | | |
| ylsulfonyl)phenyl)pyrimidin- | | |
| 2-amine Hydrochloride | | |
| 4-{5-[2-(2-Fluoro-5- | 25 | 240 |
| hydroxy-phenyl)-vinyl]- | | |
| pyrimidin-2-ylamino}-N-(2- | | |
| pyrrolidin-1-yl-ethyl)- | | |
| benzenesulfonamide | | |
| 3-((E)-2-(2-(4-(2-(pyrrolidin- | 30 | 297 |
| 1- | | |
| yl)ethoxy)phenylamino)pyri- | | |
| midin-5-yl)vinyl)phenol | | |
| trifluoroacetate | | |
| 4-{5-[2-(3-Hydroxy-phenyl)- | 39 | 436 |
| vinyl]-4-methyl-pyrimidin-2- | | |
| ylamino}-N-(2-pyrrolidin-1- | | |
| yl-ethyl)- | | |
| benzenesulfonamide | | |
| N-(3 -((E)-2-(2-(4-(piperidin- | 17 | 1278 |
| 4- | | |
| ylsulfonyl)phenylamino)pyri- | | |
| midin-5-yl)vinyl)phenyl)-3- | | |
| (trifluoromethyl)benzamide | | |
| Hydrochloride | | |
| 5-((E)-2-(1H-indol-4- | 6.3 | 154 |
| yl)vinyl)-N-(4-(piperidin-4- | | |
| 2-amine | | |
| Benzoic acid 3-(2-{2-[4-(2- | | |
| pyrrolidin-1-yl- | | |
| ethylsulfamoyl)- | | |
| phenylamino]-pyrimidin-5- | | |
| yl}-vinyl)-phenyl ester | | |
| 2-Fluoro-5-(2-{2-[4- | 12 | 213 |
| (piperidine-4-sulfonyl)- | | |
| phenylamino]-pyrimidin-5- | | |
| yl}-vinyl)-phenol | | |
| 3-(2-{2-[4-(Piperazine-1- | 4.1 | 37 |
| sulfonyl)-phenylamino]- | | |
| pyrimidin-5-yl}-vinyl)- | | |
| phenol | | |
| 4-((E)-2-(2-(4-(piperidin-4- | 25 | 40 |
| ylsulfonyl)phenylamino)pyri- | | |
| midin-5-yl)vinyl)benzene- | | |
| 1,2-diol Hydrochloride | | |
| 5-[(E)-2-(1H-indol-4- | | |
| pyrimidin-2-amine | | |
| {5-[2-(1H-Indol-4-yl)-vinyl]- | 86 | 2596 |
| pyrimidin-2-yl}-(3- | | |
| piperazin-1-yl-phenyl)-amine | | |
| 2-(4-(3-(5-((E)-2-(1H-indol- | 178 | 10000 |
| 4-yl)vinyl)pyrimidin-2- | | |
| ylamino)phenyl)piperazin-1- | | |
| yl)ethanol Hydrochloride | | |
| N-(3-{2-[2-(Pyridin-3- | 148 | >10000 |
| ylamino)-pyrimidin-5-yl]- | | |
| vinyl}-phenyl)-3- | | |
| trifluoromethyl-benzamide | | |
| 2-(4-(4-(5-((E)-2-(1H-indol- | 8.4 | 202 |
| 4-yl)vinyl)pyrimidin-2- | | |
| ylamino)phenylsulfonyl)piper- | | |
| idin-1-yl)ethanol | | |
| Hydrochloride | | |
| N-[4-Chloro-3-(2-{2-[4- | 12 | 2000 |
| (piperidine-4-sulfonyl)- | | |
| phenylamino]-pyrimidin-5- | | |
| yl}-vinyl)-phenyl]-3- | | |
| trifluoromethyl-benzamide | | |
| {5-[2-(1H-Indol-5-yl)-vinyl]- | 37 | 138 |
| pyrimidin-2-yl}-[4- | | |
| (piperidine-4-sulfonyl)- | | |
| phenyl]-amine | | |
| 4-(5-((E)-2-(1H-indol-4- | 174 | >10,000 |
| yl)vinyl)pyrimidin-2- | | |
| ylamino)benzonitrile | | |
| 4-({5-[(E)-2-(1H-indol-4- | 26 | 738 |
| yl)vinyl]pyrimidin-2- | | |
| yl}amino)--N-[4-(2- | | |
| pyrrolidin-1- | | |
| ylethyl)benzenesulfonamide | | |
| 5-[(E)-2-(1H-indol-4- | 41 | 852 |
| yl)vinyl]-N-{4-[(4- | | |
| methylpiperazin-1- | | |
| yl)carbonyl] | | |
| phenyl}pyrimidin-2-amine | | |
| 5-[(E)-2-(1H-indol-4- | 13 | 386 |
| pyrrolidin-1- | | |
| ylpropyl)sulfonyl]phenyl} | | |
| pyrimidin-2-amine | | |
| 5-[(E)-2-(1H-indol-4- | 784 | ˜10000 |
| yl)vinyl]-N-{3-[(4- | | |
| methylpiperazin-1- | | |
| yl)sulfonyl]phenyl} | | |
| pyrimidin-2-amine | | |
| 5-[(E)-2-(1H-indol-4- | 22 | 450 |
| yl)vinyl]-N-{4-(piperazin-1- | | |
| ylsulfonyl)phenyl]pyrimidin- | | |
| 2-amine | | |
| 5-[(E)-2-(1H-indol-4- | 244 | ˜10000 |
| yl)vinyl]-N-{3-(piperazin-1- | | |
| ylsulfonyl)phenyl]pyrimidin- | | |
| 2-amine | | |
| {5-[2-(1H-Indol-4-yl)-vinyl]- | 42 | 1324 |
| pyrimidin-2-yl}-(4-piperidin- | | |
| 4-yl-phenyl)-amine | | |
| 4-(2-{2-[4-(Piperidine-4- | 127 | 2270 |
| sulfonyl)-phenylamino]- | | |
| pyrimidin-5-yl}-vinyl)-1,3- | | |
| dihydro-indol-2-one, | | |
| hydrogen chloride | | |
| 5-((E)-2-(1H-indazol-4- | 1.5 | 29 |
| ylsulfonyl)phenyl)pyrimidin- | | |
| yl)vinyl)-N-(4-(piperidin-4- | | |
| 2-amine trifluoroacetic acid | | |
| salt | | |
| {5-[2-(1H-Indazol-5-yl)- | 163 | 262 |
| vinyl]-pyrimidin-2-yl}-[4- | | |
| (piperidine-4-sulfonyl)- | | |
| phenyl]-amine | | |
| 5-((E)-2-(1H- | 3140 | 2491 |
| benzo[d]imidazol-5- | | |
| yl)vinyl)-N-(4-(piperidin-4- | | |
| ylsulfonyl)phenyl)pyrimidin | | |
| 2-amine. Triflate | | |
| 5-(3-(difluoromethyl)styryl)- | 162 | 486 |
| N-(4-piperidin-4- | | |
| ylsulfonyl)phenyl)pyrimidin | | |
| 2-amine. Triflate | | |
| 6-[(E)-2-(2-{[4-(piperidin-4- | 50 | 58 |
| ylsulfonyl)phenyl]amino}pyr- | | |
| imidin-5-yl)vinyl]-1,3- | | |
| benzothiazol-2-amine | | |
| trifluoroacetic acid salt | | |
| 3-(2-{2-[4-(Piperidine-4- | 3670 | 53000 |
| sulfonyl)-phenylamino]- | | |
| pyrimidin-5-yl}-vinyl)- | | |
| benzamidine | | |
| 5-((E)-2-(6-aminopyridin-2- | 56 | 1210 |
| yl)vinyl)-N-(4-(piperidin-4- | | |
| ylsulfonyl)phenyl)pyrimidin- | | |
| 2-amine Hydrochloride | | |
| N-[4-(piperidin-4- | 38 | 163 |
| ylsulfonyl)phenyl]-5-[(E)-2- | | |
| (1H-pyrrolo[2,3-b]pyridin-4- | | |
| yl)vinyl]pyrimidin-2-amine | | |
| 3-(2-{2-[4-(Piperidine-4- | 198 | 2070 |
| sulfonyl)-phenylamino]- | | |
| pyrimidin-5-yl}-vinyl)- | | |
| benzenesulfonamide | | |
| Hydrochloride | | |
| 5-(4-(difluoromethyl)styryl)- | 429 | 1330 |
| N-(4-(piperidin-4- | | |
| ylsulfonyl)phenyl)pyrimidin- | | |
| 2-amine. Triflate | | |
| 5-(3-(trifluoromethyl)styryl)- | 565 | 3200 |
| N-(4-(piperidin-4- | | |
| ylsulfonyl)phenyl)pyrimidin- | | |
| 2-amine. TFA | | |
| 5-(3-(methyl)styryl)-N-(4- | 51 | 562 |
| (piperidin-4- | | |
| ylsulfonyl)phenyl)pyrimidin- | | |
| 2-amine. TFA | | |
| 5-[(E)-2-(1H-indol-4- | 21 | 820 |
| yl)vinyl]-N-[4-(piperidin-4- | | |
| yloxy)phenyl]pyrimidin-2- | | |
| amine trifluoroacetic acid | | |
| salt | | |
| 5-[(E)-2-(1H-indazol-4- | 3.9 | 124 |
| yl)vinyl]-N-{4-[(3- | | |
| pyrrolidin-1- | | |
| ylpropyl)sulfonyl] | | |
| phenyl}pyrimidin-2-amine | | |
| 6-{(E)-2-[2-({4-[(3- | 81 | 173 |
| pyrrolidin-1- | | |
| ylpropyl)sulfonyl]phenyl}amino) | | |
| pyrimidin-5-yl]vinyl}- | | |
| 1,3-benzothiazol-2-amine | | |
| trifluoroacetic acid salt | | |
| 5-[(E)-2-(1H-indazol-4- | 7.9 | 169 |
| yl)vinyl]-N-[4-(piperidin-4- | | |
| yloxy)phenyl]pyrimidin-2- | | |
| amine trifluoroacetic acid | | |
| salt | | |
| 6-[(E)-2-(2-{[4-(piperidin-4- | 259 | 428 |
| yloxy)phenyl]amino}pyrimid- | | |
| in-5-yl)vinyl]-1,3- | | |
| benzothiazol-2-amine | | |
| 6-[(E)-2-(2-{[4-(piperidin-4- | 192 | 169 |
| ylsulfonyl)phenyl]amino}pyr- | | |
| imidin-5-yl)vinyl]-1H- | | |
| benzimidazol-2-amine | | |
| (E)-6-(2-(2-(4-(piperazin-1- | 40 | 38 |
| ylsulfonyl)phenylamino)pyri- | | |
| midin-5- | | |
| yl)vinyl)benzo[d]thiazol-2- | | |
| amine | | |
| (3-((E)-2-(2-(4-(piperidin-4- | 73 | 517 |
| ylsulfonyl)phenylamino)pyri- | | |
| midin-5- | | |
| yl)vinyl)phenyl)methanol | | |
| hydrochloride | | |
| 2,2,2-Trifluoro-N-{6-[(E)-2- | 1130 | 475 |
| (2-{[4-(piperidin-4- | | |
| ylsulfonyl)phenyl] | | |
| amino}pyrimidin-5- | | |
| yl)vinyl]imidazolo[1,2- | | |
| a]pyridin-2-yl}acetamide | | |
| trifluoroacetic acid salt | | |
| 6-[(E)-2-(2-{[4-(piperidin-4- | 469 | 136 |
| ylsulfonyl)phenyl]amino}pyr- | | |
| imidin-5- | | |
| yl)vinyl]imidazolo[1,2- | | |
| a]pyridin-2-amine | | |
| trifluoroacetic acid salt | | |
| (E)-1-(4-(5-(2-(2- | 922 | 317 |
| aminobenzo[d]thiazol-6- | | |
| yl)vinyl)pyrimidin-2- | | |
| ylamino)phenylsulfonyl)piper- | | |
| idin-4-ol | | |
| 7-((E)-2-(2-(4-(piperidin-4- | 130 | 254 |
| ylsulfonyl)phenylamino)pyri- | | |
| midin-5-yl)vinyl)-5- | | |
| (trifluoromethyl)-1H- | | |
| benzo[d]imidazol-2-amine | | |
| 3-((E)-2-(2-(4-(piperidin-4- | 210 | 522 |
| ylsulfonyl)phenylamino)pyri- | | |
| midin-5-yl)vinyl)-5- | | |
| (trifluoromethyl)benzene | | |
| 1,2-diamine. | | |
| 4-{5-[2-(3-Hydroxy-phenyl)- | 4.6 | 36 |
| vinyl]-pyrimidine-2- | | |
| ylamino}-N-piperdin-4-yl- | | |
| benzenesulfonamide | | |
| Hydrochloride | | |
| N-(6-((E)-2-(2-(4-(piperidin- | 967 | 332 |
| 4- | | |
| ylsulfonyl)phenylamino)pyri- | | |
| midin-5- | | |
| (trifluoromethyl)benzamide | | |
| Hydrochloride | | |
| (1-{[4-({5-[(E)-2-(2-amino- | 437 | 194 |
| yl)vinyl]pyrimidin-2- | | |
| amino)phenyl]sulfonyl} | | |
| piperidin-4-yl)methanol | | |
| N-[4-(piperazin-1- | 3800 | 1230 |
| ylsulfonyl)phenyl]-5-{(E)-2- | | |
| [4-(1H-pyrazol-4- | | |
| yl)phenyl]vinyl}pyrimidin-2- | | |
| amine | | |
| 6-[(E)-2-(2-{[4-(3- | 5150 | 9450 |
| pyrrolidin-1- | | |
| ylpropyl)phenyl]amino}pyri- | | |
| midin-5-yl)vinyl]-1,3- | | |
| benzothiazol-2-amine | | |
| (E)-1-(4-(5-(2-(2- | 432 | 214 |
| aminobenzo[d]thiazol-6- | | |
| yl)vinyl)pyrimidin-2- | | |
| ylamino)phenylsulfonyl)piper- | | |
| idin-3-ol | | |
| 4-{5-[2-(3-Hydroxy-phenyl)- | 8.9 | 112 |
| vinyl]-pyrimidin-2- | | |
| ylamino}-N-(3-hydroxy- | | |
| propyl)-benzenesulfonamide | | |
| 6-[(E)-2-(2-{[4-(piperidin-4- | 794 | 899 |
| ylsulfonyl)phenyl]amino}pyr- | | |
| imidin-5-yl)vinyl]-1,2- | | |
| benzisoxazol-3-amine | | |
| 7-[(E)-2-(2-{[4-(piperidin-4- | 4.7 | 201 |
| ylsulfonyl)phenyl]amino}pyr- | | |
| imidin-5-yl)vinyl]-1,2- | | |
| benzisoxazol-3-amine | | |
| 5-((E)-(4-(1H-imidazol-2- | 374 | 1290 |
| yl)styryl))-N-(4-(piperidin-4- | | |
| ylsulfonyl)phenyl)pyrimidin- | | |
| 2-amine hydrochloride | | |
| [1-(4-{5-[2-(2-Amino-indan- | 13700 | 2790 |
| 5-yl)-vinyl]-pyrimidin-2- | | |
| ylamino}-benzenesulfonyl)- | | |
| piperidin-4-yl]-methanol | | |
| 5-((E)-2-(6- | 130 | 70 |
| (trifluoromethyl)-1H- | | |
| benzo[d][1,2,3]triazol-4- | | |
| yl)vinyl)-N-(4-(piperidin-4- | | |
| ylsulfonyl)phenyl)pyrimidin- | | |
| 2-amine | | |
| 5-((E)-2-(6- | 245 | 666 |
| (trifluoromethyl)-1H- | | |
| benzo[d]imidazol-4- | | |
| yl)vinyl)-N-(4-(piperidin-4- | | |
| ylsulfonyl)phenyl)pyrimidin- | | |
| 2-amine. | | |
| 5-((E)-3-(1H-imidazol-2- | 159 | 2720 |
| yl)styryl))-N-(4-(piperidin-4- | | |
| ylsulfonyl)phenyl)pyrimidin- | | |
| 2-amine | | |
| 2-{[5-({5-[(E)-2-(2-amino- | 154 | 156 |
| 1,3-benzothiazol-6- | | |
| yl)vinyl]pyrimidin-2- | | |
| yl}amino)-2-(piperazin-1- | | |
| ylsulfonyl)phenyl]amino} | | |
| ethanol | | |
| 2-{[5-({5-[(E)-2-(1H- | 6.5 | 233 |
| indazol-4- | | |
| yl)vinyl]pyrimidin-2- | | |
| yl}amino)-2-(piperazin-1- | | |
| ylsulfonyl)phenyl]amino} | | |
| ethanol | | |
| 4-{5-[2-(3-Hydroxy-phenyl)- | 12 | 60 |
| vinyl]-pyrimidine-2- | | |
| ylamino}-N-piperdin-3-yl- | | |
| benzenesulfonamide | | |
| Hydrochloride | | |
| 3-(2-{2-[4-(4- | 22 | 225 |
| Hydroxymethyl-piperidine- | | |
| 1-sulfonyl)-phenylamino]- | | |
| pyrimidin-5-yl}-vinyl)- | | |
| phenol | | |
| 3-[2-(2-{4-[4-(2-Hydroxy- | 18 | 160 |
| ethyl)-piperidine-1-sulfonyl]- | | |
| phenylamino}-pyrimidin-5- | | |
| yl)-vinyl]-phenol | | |
| (E)-6-(2-(2-(4-(1,4-diazepan- | 39 | 22 |
| 1- | | |
| ylsulfonyl)phenylamino)pyri- | | |
| midin-5- | | |
| yl)vinyl)benzo[d]thiazol-2- | | |
| amine | | |
| 5-((E)-4-(1H-tetrazol-5- | 3260 | 4180 |
| ylsulfonyl)phenyl)pyrimidin- | | |
| yl)styryl)-N-(4-(piperidin-4- | | |
| 2-amine hydrochloride | | |
| 5-((E)-2-(1-indanol-5- | 33 | 805 |
| yl)vinyl)-N-(4-(piperidin-4- | | |
| ylsulfonyl)phenyl)pyrimidin- | | |
| 2-amine | | |
| 5-(3-(1H-pyrazol-4- | 1740 | 1980 |
| yl)styryl)-N-(4-(piperidin-4- | | |
| ylsulfonyl)phenyl)- | | |
| pyrimidin-2-amine | | |
| Hydrochloride | | |
| 4-{5-[2-(1H-Indazol-4-yl)- | 7.6 | 133 |
| vinyl]-pyrimidin-2- | | |
| ylamino}-N-(2-pyrrolidin-1- | | |
| yl-ethyl)- | | |
| benzenesulfonamide | | |
| 5-((E)-4-(1H-tetrazol-5- | 305 | 1880 |
| yl)styryl)-N-(4-(piperidin-4- | | |
| ylsulfonyl)phenyl)pyrimidin- | | |
| 2-amine hydrochloride | | |
| 2-{[5-({5-{(E)-2-(3-amino- | 34 | 1860 |
| 1,2-benzisoxazol-7- | | |
| yl)vinyl]pyrimidin-2- | | |
| yl}amino)-2-(piperazin-1- | | |
| ylsulfonyl)phenyl]amino} | | |
| ethanol | | |
| 3-{(E)-2-[2-({3-[(2- | 7.3 | 78 |
| hydroxyethyl)amino]-4- | | |
| (piperazin-1- | | |
| ylsulfonyl)phenyl} | | |
| amino)pyrimidin-5- | | |
| yl]vinyl}phenol | | |
| trifluoroacetic acid salt | | |
| 2-{[5-({5-[(E)-2- | 131 | 2500 |
| phenylvinyl]pyrimidin-2- | | |
| yl}amino)-2-(piperazin-1- | | |
| ylsulfonyl)phenyl]amino} | | |
| ethanol trifluoroacetic acid salt | | |
| 4-({5-[(E)-2-(2-amino-1,3- | 585 | 354 |
| benzothiazol-6- | | |
| yl)vinyl]pyrimidin-2- | | |
| yl}amino)-N-(2- | | |
| hydroxyethyl)-2-piperazin-1- | | |
| ylbenzenesulfonamide | | |
| N-(2-hydroxyethyl)-4-({5- | 30 | 81 |
| [(E)-2-(1H-indazol-4- | | |
| yl)vinyl]pyrimidin-2- | | |
| yl}amino)-2-piperazin-1- | | |
| ylbenzenesulfonamide | | |
| trifluoroacetic acid salt | | |
| 5-((E)-2-(1H- | 75 | 467 |
| benzo[d][1,2,3]triazol-5- | | |
| yl)vinyl)-N-(4-(piperidin-4- | | |
| ylsulfonyl)phenyl)pyrimidin- | | |
| 2-amine | | |
| N-(4-(piperidin-4- | 1390 | 368 |
| ylsulfonyl)phenyl)-5- | | |
| vinylpyrimidin-2-amine | | |
| trifluoroacetate | | |
| (5-{2-[4-(1H-Imidazol-4-yl)- | 2860 | 5040 |
| phenyl]-vinyl}-pyrimidin-2- | | |
| yl)-[4-(piperazine-1- | | |
| sulfonyl)-phenyl]-amine | | |
| {5-[2-(1H-Indazol-4-yl)- | 44 | 289 |
| vinyl]-pyrimidin-2-yl}-[4- | | |
| (piperazine-1-sulfonyl)- | | |
| phenyl]-amine | | |
| N-[6-(2-{2-[4-(Piperazine-1- | 5110 | 5600 |
| sulfonyl)-phenylamino]- | | |
| pyrimidin-5-yl]-vinyl)- | | |
| benzothiazol-2-yI]-3- | | |
| trifluoromethyl-benzamide | | |
| N-(4-(1-methylpiperidin-4- | 4250 | 1570 |
| ylsulfonyl)phenyl)-5- | | |
| vinylpyrimidin-2-amine | | |
| 4-{5-[2-(1H-Indol-4-yl)- | 12 | 282 |
| vinyl]-pyrimidin-2- | | |
| ylamino}-N-piperidin-4-yl- | | |
| benzenesulfonamide | | |
| N-(4-(1-methylpiperidin-4- | 32 | 2040 |
| ylsulfonyl)phenyl)-5-((E)-2- | | |
| (1H-indazol-4- | | |
| yl)vinyl)pyrimidin-2-amine | | |
| N-(2-hydroxyethyl)-4-({5- | 66 | 514 |
| [(E)-2-(3- | | |
| hydroxyphenyl)vinyl]pyrimidin- | | |
| 2-yl}amino)-2-piperazin- | | |
| 1-ylbenzenesulfonamide | | |
| trifluoroacetic acid salt | | |
| N-(2-hydroxyethyl)-4-({5- | 406 | 7340 |
| [(E)-2- | | |
| phenylvinyl]pyrimidin-2- | | |
| yl}amino)-2-piperazin-1- | | |
| ylbenzenesulfonamide | | |
| trifluoroacetic acid salt | | |
| 4-{5-[2-(2-Amino- | 47 | 29 |
| benzothiazol-6-yl)-vinyl]- | | |
| piperidin-4-yl- | | |
| benzenesulfonamide | | |
| 4-{5-[2-(1H-Indazol-4-yl)- | 6.2 | 176 |
| vinyl]-pyrimidin-2- | | |
| ylamino}-N-piperidin-4-yl- | | |
| benzenesulfonamide | | |
| hydrochloride | | |
| 5-((E)-2-(7-chloro-1H-indol- | 15 | 156 |
| 4-yl)vinyl)-N-(4-(piperidin- | | |
| ylsulfonyl)phenyl)pyrimidin- | | |
| 2-amine Hydrochloride | | |
| 5-((E)-2-(7-methyl-1H-indol- | 58 | 167 |
| 4-yl)vinyl)-N-(4-(piperidin- | | |
| ylsulfonyl)phenyl)pyrimidin- | | |
| 2-amine Hydrochloride | | |
| [4-([1,4]Diazepane-1- | 13 | 195 |
| sulfonyl)-phenyl]-{5-[2-(1H- | | |
| indol-4-yl)-vinyl]-pyrimidin- | | |
| 2-yl}-amine | | |
| 5-((E)-2-(7-fluoro-1H-indol- | 8.0 | 149 |
| 4-yl)vinyl)-N-(4-(piperidin- | | |
| ylsulfonyl)phenyl)pyrimidin- | | |
| 2-amine Hydrochloride | | |
| N-[3-piperazin-1-yl-4- | 22200 | 4640 |
| (piperazin-1- | | |
| ylsulfonyl)phenyl]-5- | | |
| vinylpyrimidin-2-amine | | |
| trifluoroacetic acid salt | | |
| 6-[(E)-2-(2-{[3-piperazin-1- | 318 | 152 |
| yl-4-(piperazin-1- | | |
| ylsulfonyl)phenyl]amino} | | |
| pyrimidin-5-yl)vinyl]-1,3- | | |
| benzothiazol-2-amine | | |
| 3-(2-{2-[4-([1,4]Diazepane- | 781 | 2440 |
| 1-sulfonyl)-phenylamino]- | | |
| pyrimidin-5-yl}-vinyl)- | | |
| benzenesulfonamide | | |
| 4-(2-{2-[4-([1,4]Diazepane- | 977 | 298 |
| 1-sulfonyl)-phenylamino]- | | |
| pyrimidin-5-yl}-vinyl)- | | |
| benzenesulfonamide | | |
| (S)-1-(4-(4-(5-((E)-2-(1H- | 43 | 464 |
| indol-4-yl)vinyl)pyrimidin-2- | | |
| ylamino)phenylsulfonyl) | 43 | |
| piperidin-1-yl)-2-aminopropan-1- | | |
| one hydrochloride | | |
| [4-([1,4]Diazepane-1- | 12 | 184 |
| sulfonyl)-phenyl]-{5-[2-(1H- | | |
| indazol-4-yl)-vinyl]- | | |
| pyrimidin-2-yl}-amine | | |
| {5-[2-(1H-Indol-4-yl)-vinyl]- | 54 | 488 |
| pyrimidin-2-yl}-[4-(3- | | |
| piperazin-1-yl-propane-1- | | |
| sulfonyl)-phenyl]-amine | | |
| N-(6-((E)-2-(2-(4-(piperidin- | 629 | 2740 |
| 4- | | |
| ylsulfonyl)phenylamino)pyri- | | |
| midin-5- | | |
| yl)vinyl)benzo[d]thiazol-2- | | |
| yl)-3-fluorobenzamide | | |
| Hydrochloride | | |
| 5-[(E)-2-(1H-indazol-4- | 20 | 336 |
| yl)vinyl]-N-[3-piperazin-1- | | |
| yl-4-piperazin-1- | | |
| ylsulfonyl)phenyl]pyrimidin- | | |
| 2-amine | | |
| 5-[(E)-2-(1H-indol-4- | 84 | 885 |
| yl)vinyl]-N-[3-piperazin-1- | | |
| yl-4-(piperazin-1- | | |
| ylsulfonyl)phenyl]pyrimidin- | | |
| 2-amine trifluoroacetic acid | | |
| salt | | |
| 4-{5-[2-(3-Hydroxy-phenyl)- | 16 | 248 |
| vinyl]-pyrimidin-2- | | |
| ylamino}-2,6,N-trimethyl-N- | | |
| (2-pyrrolidin-1-yl-ethyl)- | | |
| benzenesulfonamide | | |
| 3-(2-{2-[2-Methyl-4- | 3450 | 10400 |
| (piperazine-1-sulfonyl)- | | |
| phenylamino]-pyrimidin-5- | | |
| yl}-vinyl)-phenol | | |
| 6-[(E)-2-(2-{[3-fluoro-4- | 100 | 94 |
| ylsulfonyl)phenyl]amino} | | |
| pyrimidin-5-yl)vinyl]-1,3- | | |
| benzothiazol-2-amine | | |
| 6-[(E)-2-(2-{[4-(piperazin-1- | 1020 | 2100 |
| ylsulfonyl)-3- | | |
| (trifluoromethyl)phenyl] | | |
| amino)pyrimidin-5- | | |
| yl)vinyl]-1,3-benzothiazol-2- | | |
| amine | | |
| 3-(2-{2-[4-(Piperazine-1- | 222 | 1410 |
| sulfonyl)-3-trifluoromethyl- | | |
| phenylamino]-pyrimidin-5- | | |
| yl}-vinyl)-phenol | | |
| 3-(2-{2-[3-Fluoro-4- | 56 | 266 |
| (piperazine-1-sulfonyl)- | | |
| phenylamino]-pyrimidin-5- | | |
| yl}-vinyl)-phenol | | |
| 3-(2-{2-[2,5-Difluoro-4- | | |
| phenylamino]-pyrimidin-5- | | |
| yl}-vinyl)-phenol | | |
| N-(4-((E)-2-(2-(4-(piperidin- | 506 | 1010 |
| 4- | | |
| ylsulfonyl)phenylamino)pyri- | | |
| midin-5-yl)vinyl)phenyl)-3- | | |
| (trifluoromethyl)benzamide | | |
| Hydrochloride | | |
| 4-((E)-2-(2-(4-(piperidin-4- | 39 | 394 |
| ylsulfonyl)phenylamino)pyri- | | |
| midin-5- | | |
| yl)vinyl)benzo[d]thiazol-2- | | |
| amine Hydrochloride | | |
| 3-(2-{2-[4-(Piperazine-1- | 97 | 752 |
| sulfonyl)-3- | | |
| trifluoromethoxy- | | |
| phenylamino]-pyrimidin-5- | | |
| yl}-vinyl)-phenol | | |
| 3-(2-{2-[4-(Piperazine-1- | 44700 | 1530000 |
| sulfonyl)-2-trifluoromethyl- | | |
| phenylamino]-pyrimidin-5- | | |
| yl}-vinyl)-phenol | | |
| 3-(2-{2-[4-(Piperidin-4- | 31 | 96 |
| ylmethanesulfonyl)- | | |
| phenylamino]-pyrimidin-5- | | |
| yl}-vinyl)-phenol | | |
| (E)-6-(2-(2-(4-(1,4-diazepan- | 30700 | 9310 |
| methylphenylamino)pyrimidin- | | |
| 5-yl)vinyl)benzo [d]thiazol- | | |
| 2-amine | | |
| (E)-6-(2-(2-(4-(1,4-diazepan- | 681 | 23 |
| 1-ylsulfonyl)-2- | | |
| methylphenylamino)pyrimidin- | | |
| 5-yl)vinyl)benzo[d]thiazol- | | |
| 2-amine | | |
| 6-[2-(2- {4-[4-(2-Methoxy- | 1150 | 380 |
| ethyl)-piperazine-1- | | |
| sulfonyl]-phenylamino}- | | |
| pyrimidin-5-yl)-vinyl]- | | |
| benzothiazol-2-ylamine | | |
| (4-(5-((E)-2-(1H-indazol-4- | 20 | 231 |
| yl)vinyl)pyrimidin-2- | | |
| ylamino)phenyl)(piperazin- | | |
| 1-yl)methanone | | |
| hydrochloride | | |
| {5-[2-(3-Methoxy-phenyl)- | 172 | 664 |
| vinyl]-pyrimidin-2-yl}-[4- | | |
| piperidine-4-sulfonyl)- | | |
| phenyl]-amine | | |
| 5-((E)-2-(1H-indazol-3- | 191 | 202 |
| yl)vinyl)-N-(4-(piperidin-4- | | |
| ylsulfonyl) | | |
| phenyl)pyrimidin-2-amine | | |
| Hydrochloride | | |
| (E)-6-(2-(2-(4-(1,4-diazepan- | 1060 | 593 |
| 1-ylsulfonyl)-3- | | |
| methylphenylamino)pyrimidin- | | |
| 5-yl)vinyl)benzo[d]thiazol- | | |
| 2-amine | | |
| [4-(Piperazine-1-sulfonyl)- | 269 | 89 |
| phenyl]-{5-[2-(6- | | |
| trifluoromethyl-1H- | | |
| benzotriazol-4-yl)-vinyl]- | | |
| pyrimidin-2-yl}-amine | | |
| 4-{5-[2-(2- | 1440 | 898 |
| Aminobenzothiazol-6-yl)- | | |
| vinyl]-pyrimidin-2- | | |
| ylamino}-N-piperidin-3-yl- | | |
| benzenesulfonamide | | |
| Hydrochloride | | |
| 6-(2-{2-[4-(Piperidin-4- | 169 | 76 |
| ylmethanesulfonyl)- | | |
| phenylamino]-pyrimidin-5- | | |
| yl}-vinyl)-benzothiazol-2- | | |
| ylamine | | |
| [4-(Piperazine-1-sulfonyl)- | 61 | 21 |
| phenyl]-{5-[2-(6-chloro-1H- | | |
| benzotriazol-4-yl)-vinyl]- | | |
| pyrimidin-2-yl}-amine | | |
| 4-({5-[(E)-2-(2-amino-1,3- | 109 | 38 |
| benzothiazol-6- | | |
| yl)vinyl}pyrimidin-2- | | |
| yl}amino)-N-[2- | | |
| (dimethylamino)ethyl]-N- | | |
| piperidin-4- | | |
| ylbenzenesulfonamide | | |
| N-[2-(dimethylamino)ethyl]- | 25 | 182 |
| 4-({5-[(E)-2-(1H-indol-4- | | |
| yl)vinyl]pyrimidin-2- | | |
| yl}amino)-N-piperidin-4- | | |
| ylbenzenesulfonamide | | |
| N-[2-(dimethylamino)ethyl]- | 10 | 113 |
| 4-({5-[(E)-2-(1H-indazol-4- | | |
| yl)vinyl]pyrimidin-2- | | |
| yl}amino)-N-piperidin-4- | | |
| ylbenzenesulfonamide | | |
| 4-{5-[2-(2-Amino- | 93 | 45 |
| benzothiazol-6-yl)- | | |
| vinyl]pyrimidin-2-ylamino}- | | |
| N-methyl-N-piperidin-4-yl- | | |
| benzensulfonamide | | |
| Hydrochloride | | |
| 6-(2-{2-[4-(Azepane-4- | 27 | 14 |
| sulfonyl)-phenylamino]- | | |
| pyrimidin-5-yl}-vinyl)- | | |
| benzothiazol-2-ylamine | | |
| 6-{(E)-2-[2-({4-[(4- | 42 | 21 |
| aminopiperidin-1- | | |
| yl)sulfonyl]phenyl}amino) | | |
| pyrimidin-5-yl]vinyl}-1,3- | | |
| benzothiazol-2-amine | | |
| N-{4-[(4-aminopiperidin-1- | 5.1 | 173 |
| yl)sulfonyl]phenyl}-5-[(E)- | | |
| 2-(1H-indazol-4- | | |
| yl)vinyl]pyrimidin-2-amine | | |
| N-{4-[(4-aminopiperidin-1- | 13 | 347 |
| yl)sulfonyl]phenyl}-5-[(E)- | | |
| 2-(1H-indol-4- | | |
| yl)vinyl]pyrimidin-2-amine | | |
| 3-((E)-2-(6-(4-(piperidin-4- | 32 | 192 |
| ylsulfonyl)phenylamino)pyri- | | |
| din-3-yl)vinyl)phenol | | |
| hydrochloride | | |
| 5-((E)-2-(6-fluoro-1H- | 7.2 | 12 |
| benzo[d][1,2,3]triazol-4- | | |
| yl)vinyl)-N-(4-(piperidin-4- | | |
| ylsulfonyl)phenyl)pyrimidin- | | |
| 2-amine trifluoroacetate | | |
| 5-((E)-2-(2-(4-(piperidin-4- | 101 | 416 |
| ylsulfonyl)phenylamino) | | |
| pyrimidin-5- | | |
| yl)vinyl)benzo[d]thiazol-2- | | |
| amine Hydrochloride | | |
| (E)-6-(2-(2-(4-(1,4-diazepan- | 10800 | 6980 |
| 1-ylsulfonyl)-2- | | |
| fluorophenylamino)pyrimidin- | | |
| 5-yl)vinyl)benzo[d]thiazol- | | |
| 2-amine hydrochloride | | |
| 5-(2-{2-[4-(Piperidine-4- | 2420 | 1800 |
| sulfonyl)-phenylaminol]- | | |
| pyrimidin-5-yl}-vinyl)- | | |
| indan-1-one oxime | | |
| [4-([1,4]Diazepane-1- | 59 | 67 |
| sulfonyl)-phenyl]-{5-[2-(6- | | |
| trifluoromethyl-1H- | | |
| benzotriazol-4-yl)-vinyl]- | | |
| pyrimidin-2-yl}-amine | | |
| 1-(4-(4-(5-((E)-2-(1H-indol- | 66 | 700 |
| 4-yl)vinyl)pyrimidin-2- | | |
| ylamino)phenylsulfonyl)piper- | | |
| idin-1-yl)-2-aminoethanone | | |
| hydrochloride | | |
| 5-(2-{2-[4-(Piperidine-4- | 593 | 3650 |
| sulfonyl)-phenylamino]- | | |
| pyrimidin-5-yl}-vinyl)- | | |
| indan-1-one oxime | | |
| 4-({5-[(E)-2-(2-amino-1,3- | 84 | 33 |
| benzothiazol-6- | | |
| vinyl]pyrimidin-2- | | |
| yl}amino)-N-(2- | | |
| hydroxyethyl)-N-piperidin-4- | | |
| ylbenzenesulfonamide | | |
| N-(2-hydroxyethyl)-4-({5- | 77 | 251 |
| [(E)-2-(1H-indazol-4- | | |
| yl)vinyl]pyrimidin-2- | | |
| yl}amino)-N-piperidin-4- | | |
| ylbenzenesulfonamide | | |
| N-(2-hydroxyethyl)-4-({5- | 430 | 728 |
| [(E)-2-(1H-indol-4- | | |
| yl)vinyl]pyrimidin-2- | | |
| yl}amino)-N-piperidin-4- | | |
| ylbenzenesulfonamide | | |
| (4-{5-[2-(2-Amino- | 360 | 87 |
| benzothiazol-6-yl)-vinyl]- | | |
| pyrimidin-2-ylamino}- | | |
| phenyl)-piperazin-1-yl- | | |
| methanone | | |
| [4-([1,4]Diazepane-1- | 41 | 16 |
| sulfonyl)-phenyl]-{5-[2-(6- | | |
| chloro-1H-benzotriazol-4- | | |
| yl)-vinyl]-pyrimidin-2-yl}- | | |
| amine | | |
| [4-({5-[(E)-2-(2-amino- | 113 | 96 |
| 1,3-benzothiazol-6- | | |
| yl)vinyl]pyrimidin- | | |
| yl}amino)phenyl]sulfonyl}- | | |
| N2-piperidin-4- | | |
| ylglycinamide | | |
| (E)-N-(3-(5-(2-(2- | 464 | 160 |
| aminobenzo[d]thiazol-6- | | |
| yl)vinyl)pyrimidin-2- | | |
| ylamino)phenyl)piperazine- | | |
| 1-sulfonamide hydrochloride | | |
| 2-[(1-{[4-({5-[(E)-2-(2- | 158 | 146 |
| amino-1,3-benzothiazol-6- | | |
| yl)vinyl]pyrimidin-2- | | |
| yl}amino)phenyl]sulfonyl} | | |
| piperidin-4-yl)amino]ethanol | | |
| 3-((E)-2-(2-(4-(piperidin-4- | 10 | 65 |
| ylsulfonyl)phenylamino)-4- | | |
| methylpyrimidin-5- | | |
| yl)vinyl)phenol | | |
| hydrochloride | | |
| 6-(2-{2-[4-(2-Morpholin-4- | 630 | 1200 |
| yl-ethylsulfanyl)- | | |
| phenylamino]-pyrimidin-5- | | |
| yl}-vinyl)-benzothiazol-2- | | |
| ylamine | | |
| 6-(2-{2-[4-(2-Morpholin-4- | 272 | 283 |
| yl-ethanesulfinyl)- | | |
| phenylamino]-pyrimidin-5- | | |
| yl}-vinyl)-benzothiazol-2- | | |
| ylamine | | |
| 4-(4-{5-[2-(2-Amino- | 233 | 237 |
| benzothiazol-6-yl)-vinyl]- | | |
| benzenesulfonyl)-piperidine- | | |
| pyrimidin-2-ylamino)- | | |
| 1-carboxylic acid ethylamide | | |
| (1-{[4-({5-[(E)-2-(2-amino- | 119 | 115 |
| 1,3-benzothiazol-6- | | |
| yl)vinyl]pyrimidin-2- | | |
| yl}amino)phenyl]sulfonyl} | | |
| piperazin-2-yl)methanol | | |

Although the invention has been described with reference to the above examples, it will be understood that modifications and variations are encompassed within the spirit and scope of the invention. Accordingly, the invention is limited only by the following claims.

What is claimed is:

1. A compound having the general structure (A): 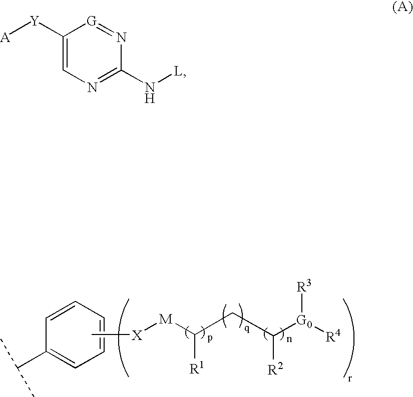
wherein L is:
wherein X is selected from a group consisting of O, C═O, SO₂, and CH₂; M is a bond or NR⁹; or X and M taken together is a bond;
each of R¹ and R² is independently selected from a group consisting of H, CF₃, F, Cl, Br, I, OH, OCH₃, CN, OCF₃, NH₂, C₁-C₆ substituted or unsubstituted alkyl, substituted or unsubstituted cycloalkyl, substituted or unsubstituted heterocycle, substituted or unsubstituted aryl, and substituted or unsubstituted heteroaryl; or R¹ and R² taken together form a bond; or R¹ and R² taken together form a moiety selected from a group consisting of (CH₂)ₘ, (CH₂)ᵣ—S(CH₂)ₘ, (CH₂)ᵣ—SO—(CH₂)ₘ, (CH₂)ᵣ—SO₂—(CH₂)ₘ, (CH₂)ᵣ—NR⁹—(CH₂)ₘ, and (CH₂)ᵣ—O—(CH₂)ₘ, wherein each of p, q, r, n, m is independently an integer having the value between 0 and 6;
R⁹ is selected from a group consisting of H, C₁-C₆ substituted or unsubstituted alkyl, C₁-C₆ substituted or unsubstituted alkenyl, C₁-C₆ substituted or unsubstituted alkynyl, C₁-C₆ substituted or unsubstituted hydroxyalkyl or aminoalkyl, C₁-C₆ substituted or unsubstituted branched alkyl, C₁-C₆ substituted or unsubstituted cycloalkyl, substituted or unsubstituted aryl connected through carbon or a heteroatom, substituted or unsubstituted heteroaryl connected through carbon or a heteroatom, C₁-C₆ alkoxy, a halogen, CF₃, —OCF₃, CHR³R⁴, SR³, SOR³, SO₂R³, SO₂NR³R⁴, SO₃R³, POR³, PO₂R³, PO₂NR³R⁴, PO₂CR³R⁴, PO₃R³, NR³R⁴, NO₂, CN, OH, CONR³R⁴, COR³, COOR³, NR³COR⁴, NR³CONR³R⁴, OCONR³R⁴, CSNR³R⁴, CSR³, NR³CSNR³R⁴, SCONR³R⁴ SCSNR³R⁴, and SCSNR³R⁴;
G₀ is selected from a group consisting of N, O, H, of CH, with the proviso that if G₀ is N, then each of R³ and R⁴ is independently selected from a group consisting of H, CF₃, F, Cl, Br, I, OH, OCH₃, CN, OCF₃, NH₂, C₁-C₆ alkyl, C₁-C₆ substituted or unsubstituted hydroxyalkyl or aminoalkyl, C₁-C₆ substituted or unsubstituted branched alkyl, substituted or unsubstituted aryl, and substituted or unsubstituted heteroaryl, or R³ and R⁴ taken together form a moiety is selected from a group consisting of (CH₂)ₘ, (CH₂)ᵣ—S—(CH₂)ₘ, (CH₂)ᵣ—SO—(CH₂)ₘ, (CH₂)ᵣ—SO₂—(CH₂)ₘ, (CH₂)ᵣ—NR⁹—(CH₂)ₘ, and (CH₂)ᵣ—O—(CH₂)ₘ;
with additional provisos that if G₀ is N, then R¹ and R⁹ taken together form a moiety selected from a group consisting of (CH₂)ₘ, (CH₂)ᵣ—S(CH₂)ₘ, (CH₂)ᵣ—SO—(CH₂)ₘ, (CH₂)ᵣ—SO₂—(CH₂)ₘ, (CH₂)ᵣ—NR⁹—(CH₂)ₘ, or (CH₂)ᵣ—O—(CH₂)ₘ; or R¹ and R⁴ taken together form a moiety selected from a group consisting of (CH₂)ₘ, (CH₂)ᵣ—S—(CH₂)ₘ, (CH₂)ᵣ—SO—(CH₂)ₘ, (CH₂)ᵣ—SO₂—(CH₂)ₘ, (CH₂)ᵣ—NR⁹—(CH₂)ₘ, or (CH₂)ᵣ—O—(CH₂)ₘ; or R⁹ and R⁴ taken together form a moiety selected from a group consisting of (CH₂)ₘ, (CH₂)ᵣ—S—(CH₂)ₘ, (CH₂)ᵣ—SO₂—(CH₂)ₘ, (CH₂)ᵣ—SO₂—(CH₂)ₘ, (CH₂)ᵣ—NR⁹—(CH₂)ₘ, and (CH₂)ᵣ—O—(CH₂)ₘ; or R³ and R⁴ taken together form a moiety selected from a group consisting of (CH₂)ₘ, (CH₂)ᵣ—S—(CH₂)ₘ, (CH₂)ᵣ—SO—(CH₂)ₘ, (CH₂)ᵣ—SO₂—(CH₂)ₘ, (CH₂)ᵣ—NR⁹—(CH₂)ₘ, and (CH₂)ᵣ—O—(CH₂)ₘ;
with a further proviso that if G₀ is O, then R³ is selected from a group consisting of H, CF₃, F, Cl, Br, I, OH, OCH₃, CN, OCF₃, NH₂, C₁-C₆ alkyl and C₁-C₆ substituted or unsubstituted hydroxyalkyl or aminoalkyl, substituted or unsubstituted branched alkyl, substituted or unsubstituted cycloalkyl, substituted heterocyclic connected through carbon or nitrogen, substituted or unsubstituted aryl, and substituted or unsubstituted heteroaryl connected through carbon or nitrogen, with no group R⁴;
R¹ and R⁹ taken together form a moiety selected from a group consisting of (CH₂)ₘ, (CH₂)ᵣ—S—(CH₂)ₘ, (CH₂)ᵣ—SO—(CH₂)ₘ, (CH₂)ᵣ—SO₂—(CH₂)ₘ, (CH₂)ᵣ—NR⁹—(CH₂)ₘ, or (CH₂)ᵣ—O—(CH₂)ₘ; or R¹ and R³ taken together form a moiety selected from a group consisting of (CH₂)ₘ, (CH₂)ᵣ—S—(CH₂)ₘ, (CH₂)ᵣ—SO—(CH₂)ₘ, (CH₂)ᵣ—SO₂—(CH₂)ₘ, (CH₂)ᵣ—NR⁹—(CH₂)ₘ, or (CH₂)ᵣ—O—(CH₂)ₘ; or R⁹ and R³ taken together form a moiety selected from a group consisting of (CH₂)ₘ, (CH₂)ᵣ—S—(CH₂)ₘ, (CH₂)ᵣ—SO—(CH₂)ₘ, (CH₂)ᵣ—SO₂—(CH₂)ₘ, (CH₂)ᵣ—NR⁹—(CH₂)ₘ, or (CH₂)ᵣ—O—(CH₂)ₘ;
with a further proviso that if G₀=CH, then each of R³ and R⁴ is independently selected from a group consisting of H, CF₃, F, Cl, Br, I, OH, OCH₃, CN, OCF₃, NH₂, C₁-C₆ alkyl, C₁-C₆ substituted or unsubstituted hydroxyalkyl or aminoalkyl, C₁-C₆ substituted or unsubstituted branched alkyl, substituted or unsubstituted aryl, C₁-C₆ substituted or unsubstituted heterocycle connected through carbon or nitrogen, and substituted or unsubstituted heteroaryl connected through carbon or nitrogen, or R³ and R⁴ taken together form a moiety selected from a group consisting of (CHR⁹)ᵣ—(CHR⁹)ₘ—(CHR⁹)ₚ, (CHR⁹)ᵣ—S—(CHR⁹)ₘ, (CHR⁹)ᵣ—SO—(CHR⁹)ₘ, (CHR⁹)ᵣ—SO₂—(CHR⁹)ₘ, (CHR⁹)ᵣ—NR⁹—(CHR⁹)ₘ, or (CHR⁹)ᵣ—O(CHR⁹)ₘ;
A is an aryl or a heteroaryl moiety selected from a group consisting of: 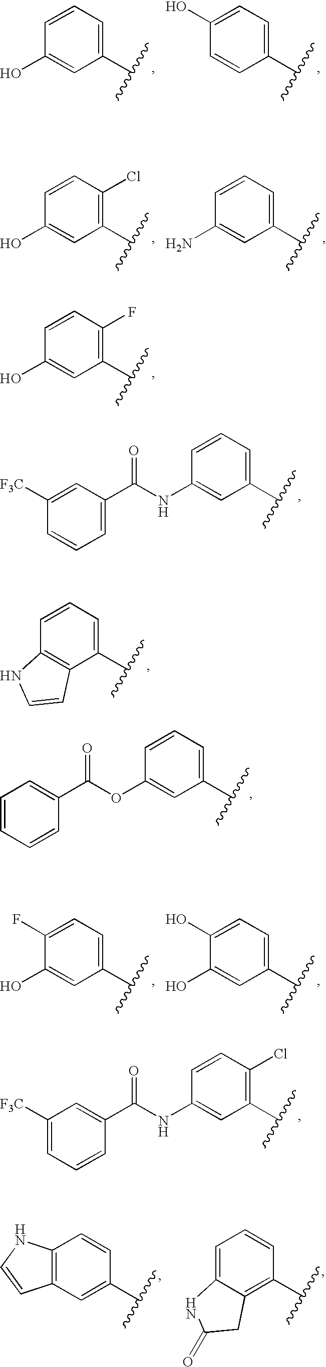 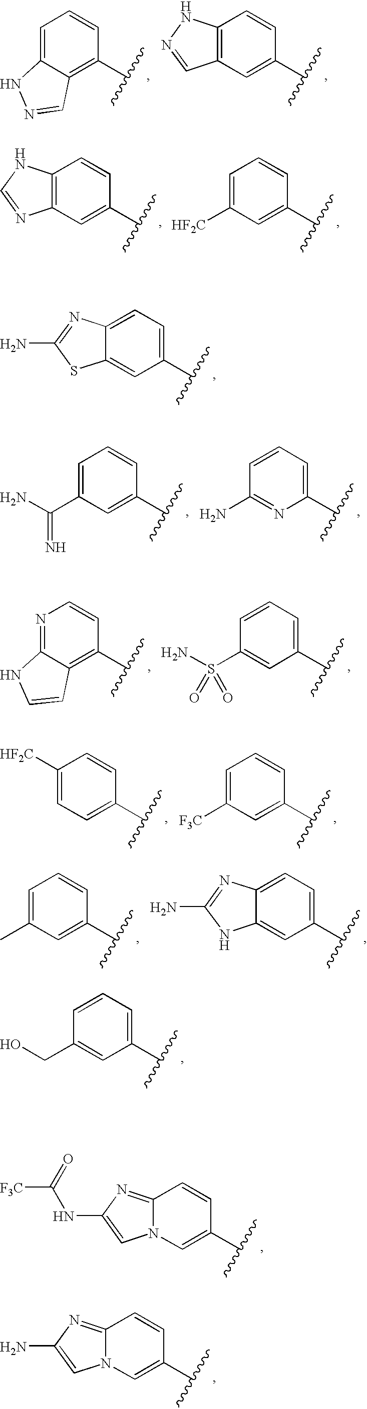 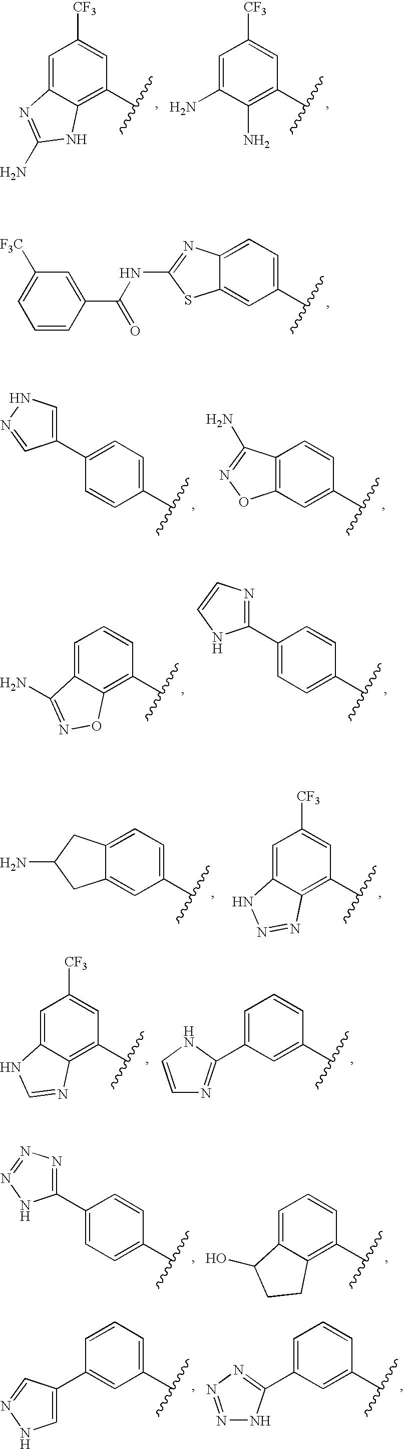 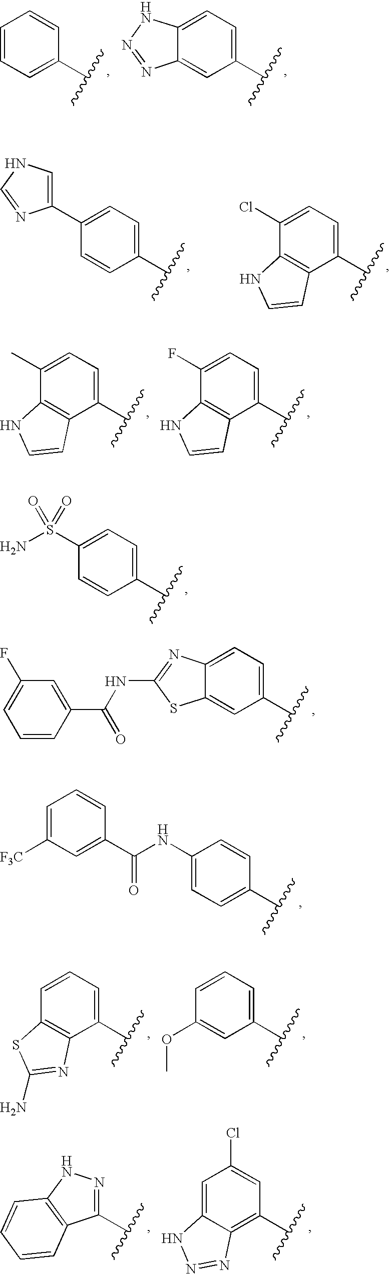 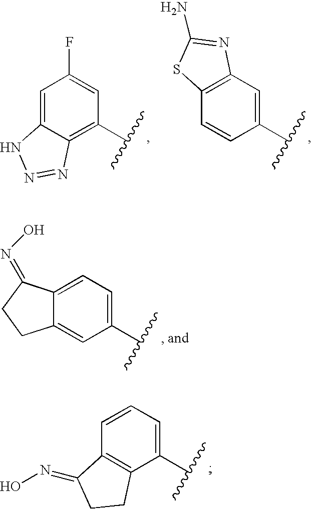
G is selected from a group consisting of N, CH and CR, wherein R is an unsubstituted or substituted lower alkyl; and
Y is a linking moiety selected from a group consisting of:

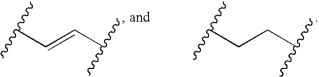

2. The compound of claim 1 or 2, wherein L is selected from a group consisting of:

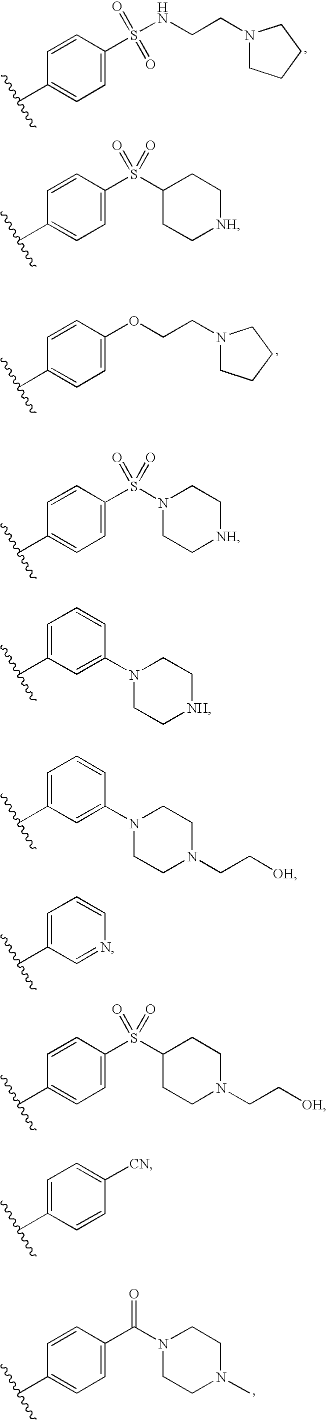

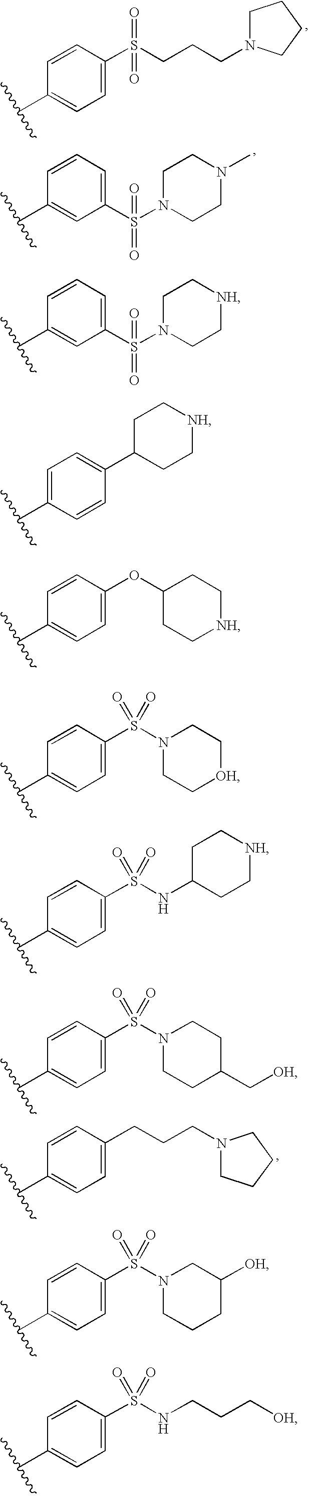

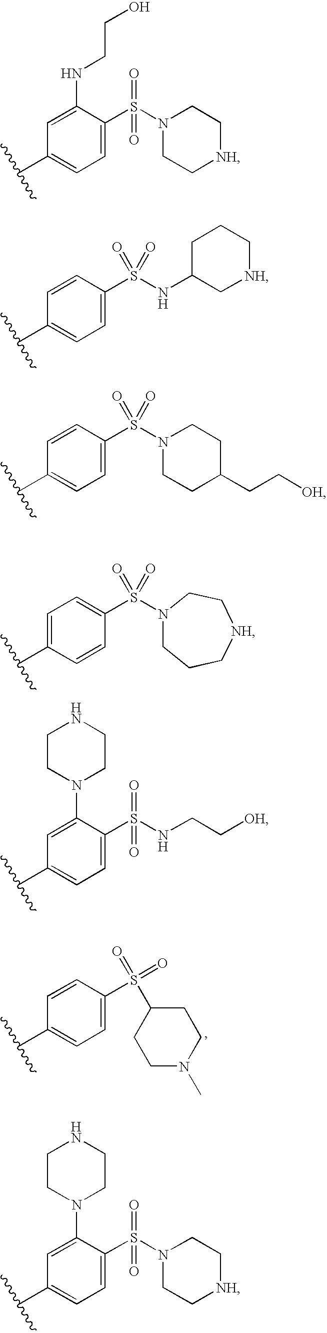

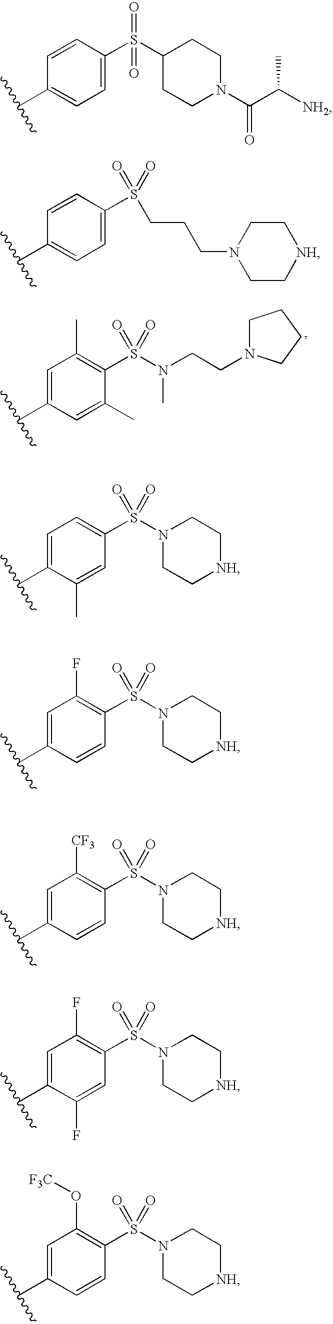

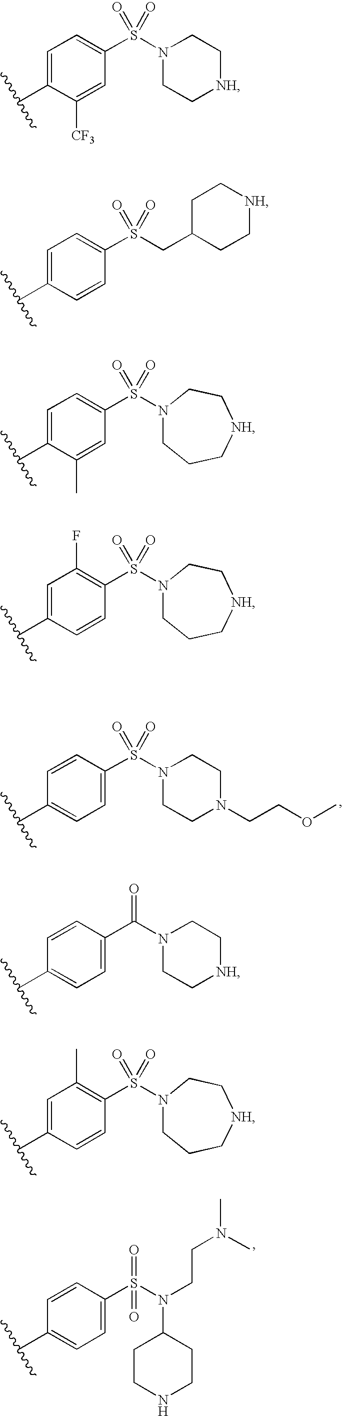

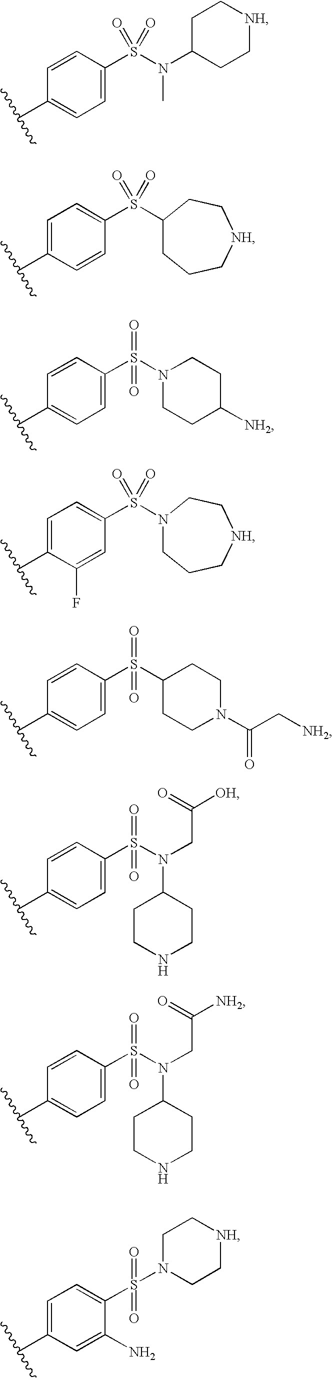

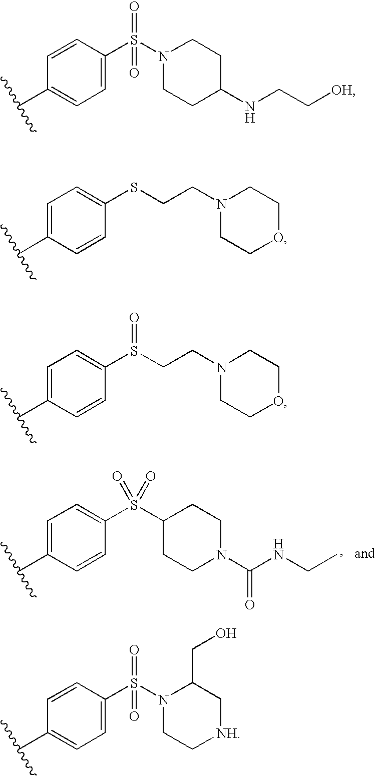

3. The compound of claim 1 or 2, wherein the compound is selected from the group consisting of compounds having formulas (I)-(CLXV):

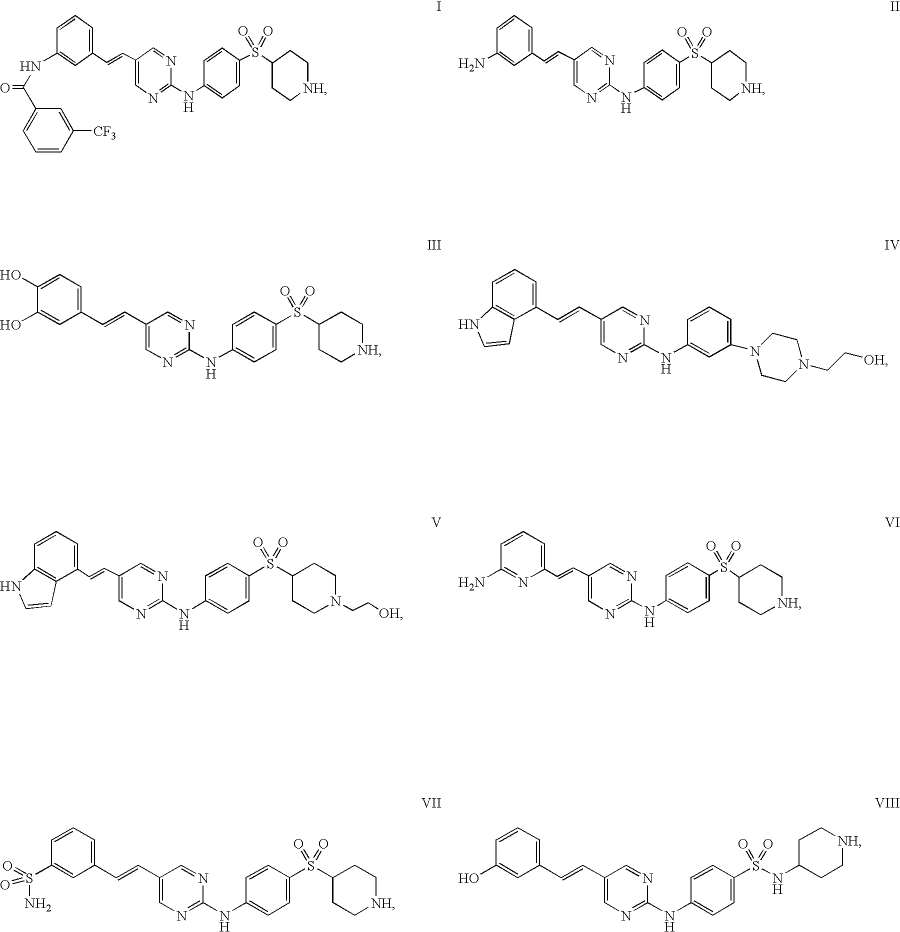

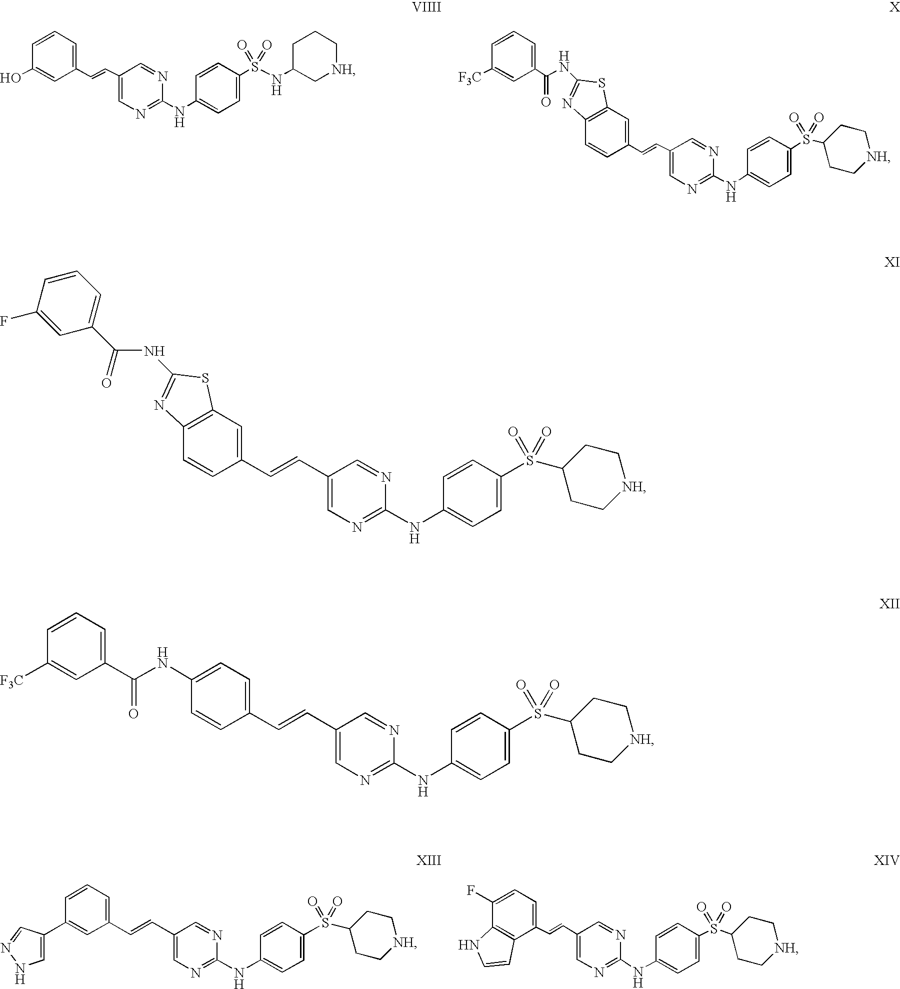

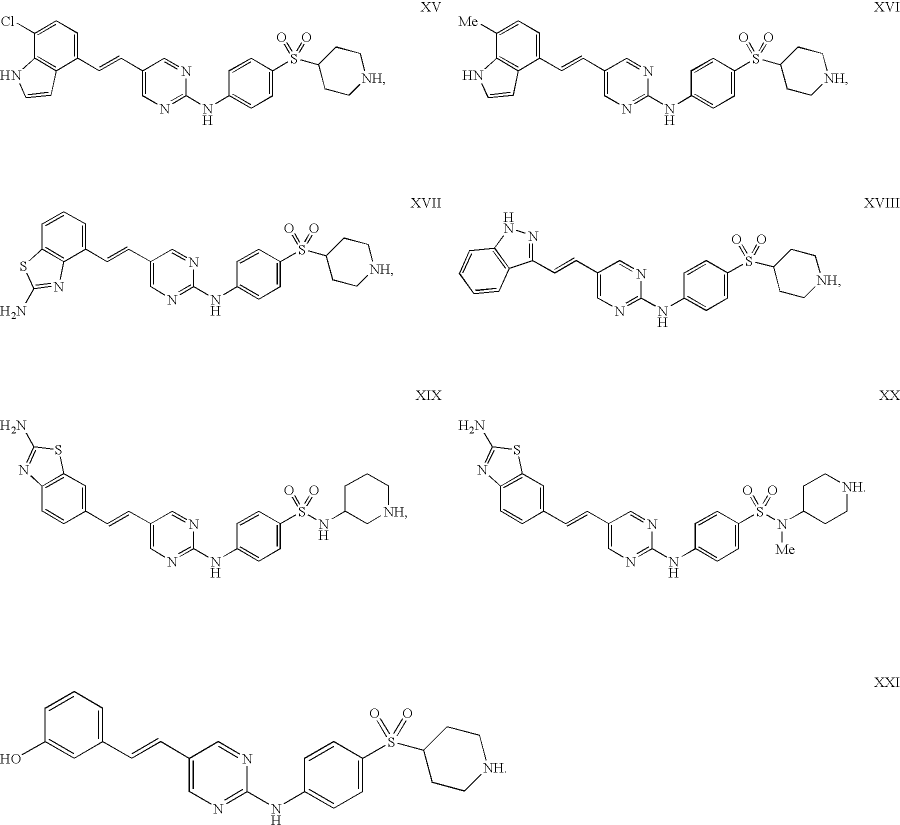

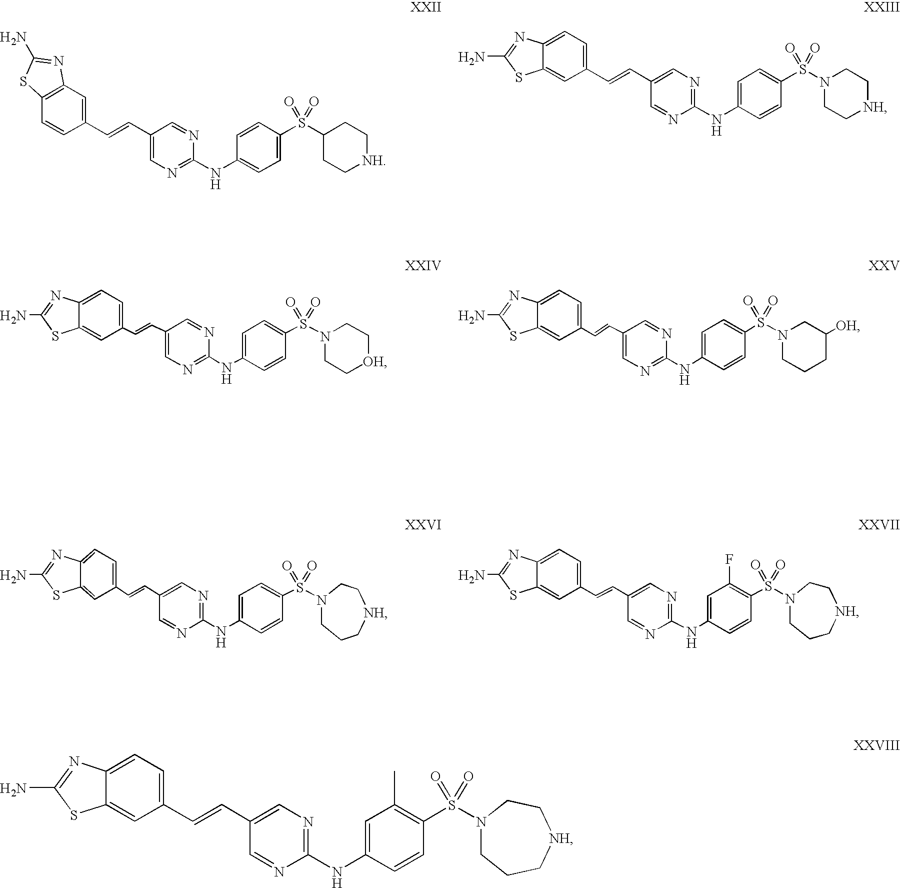

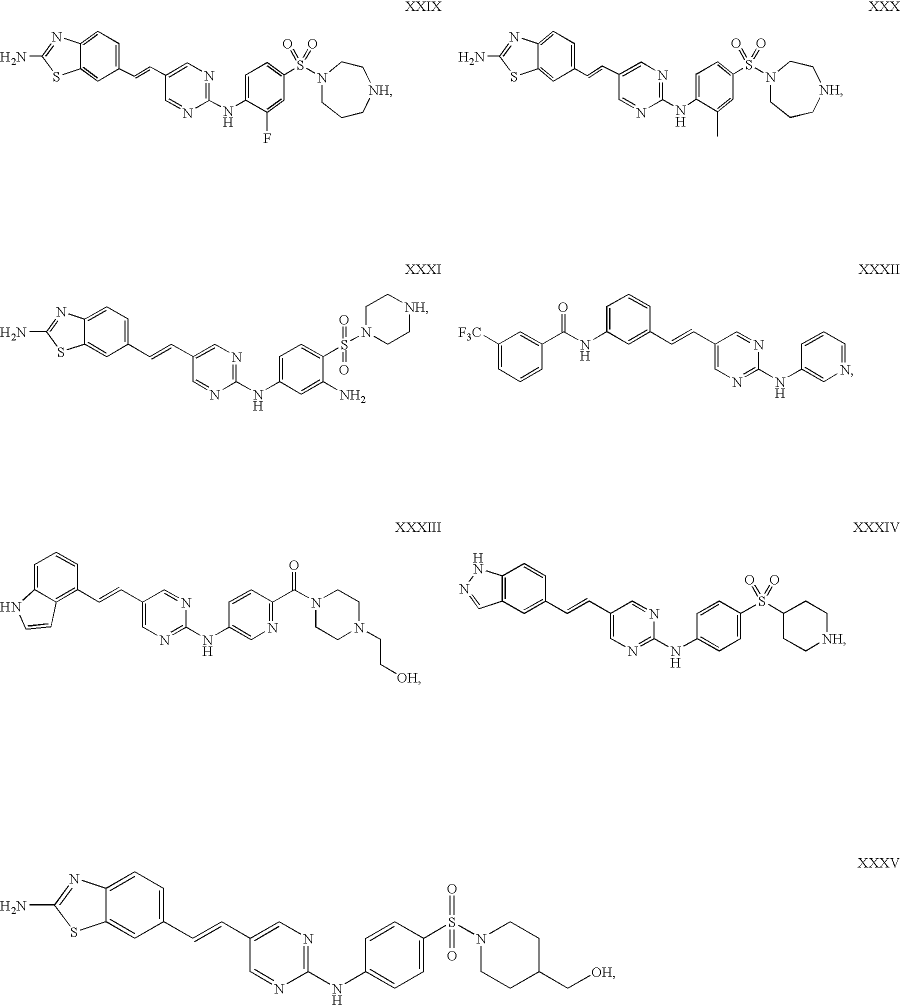

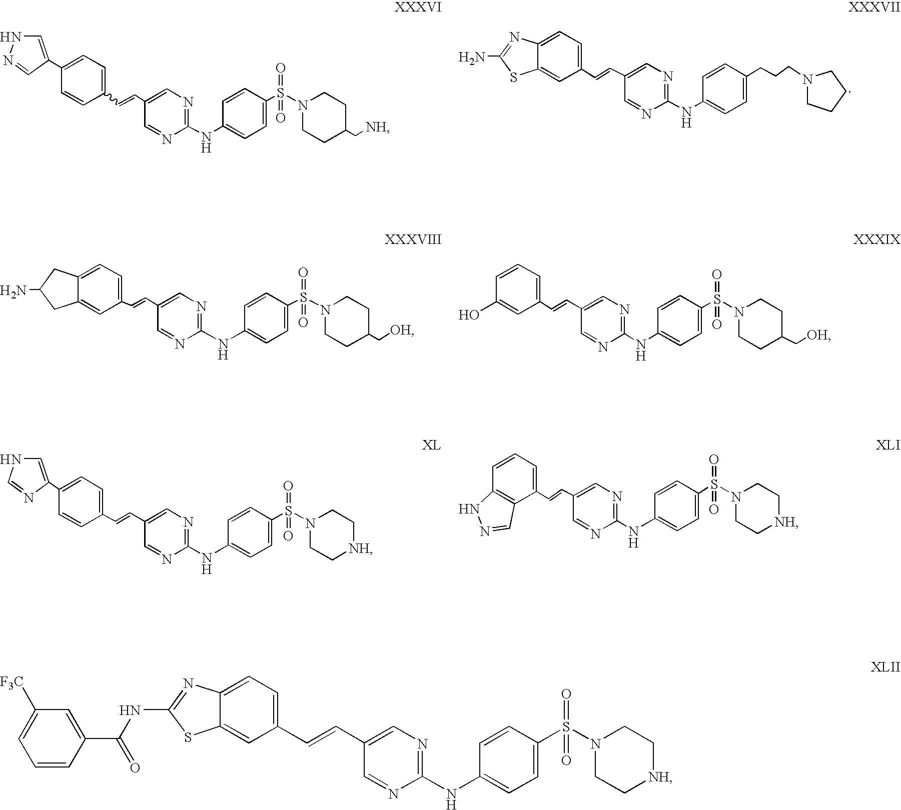

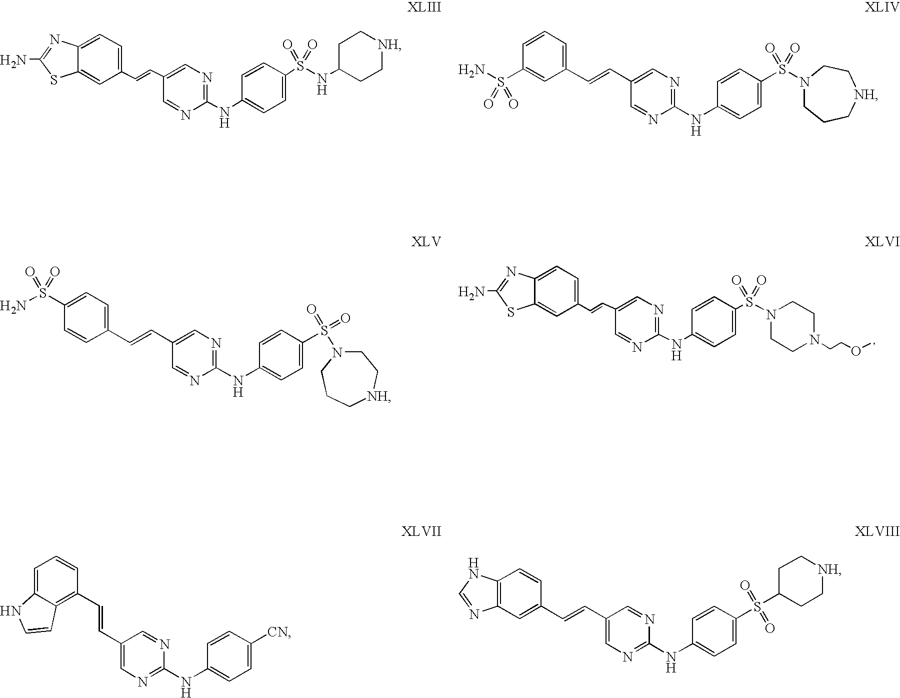

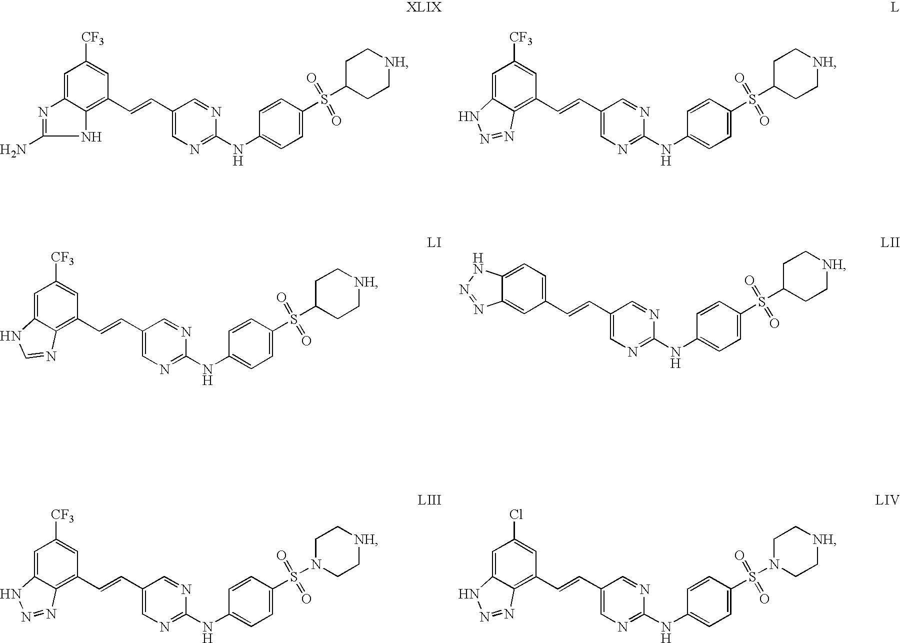

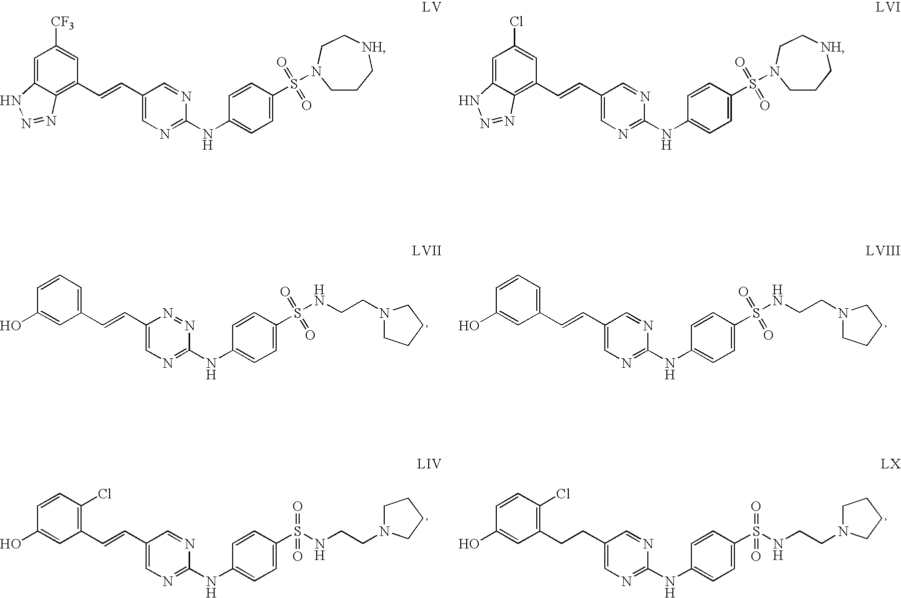

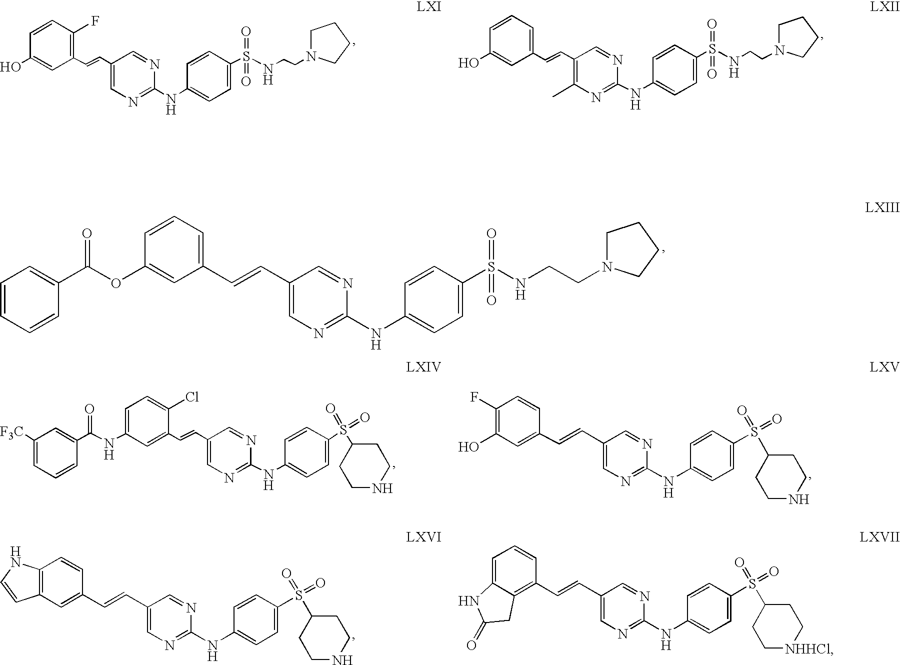

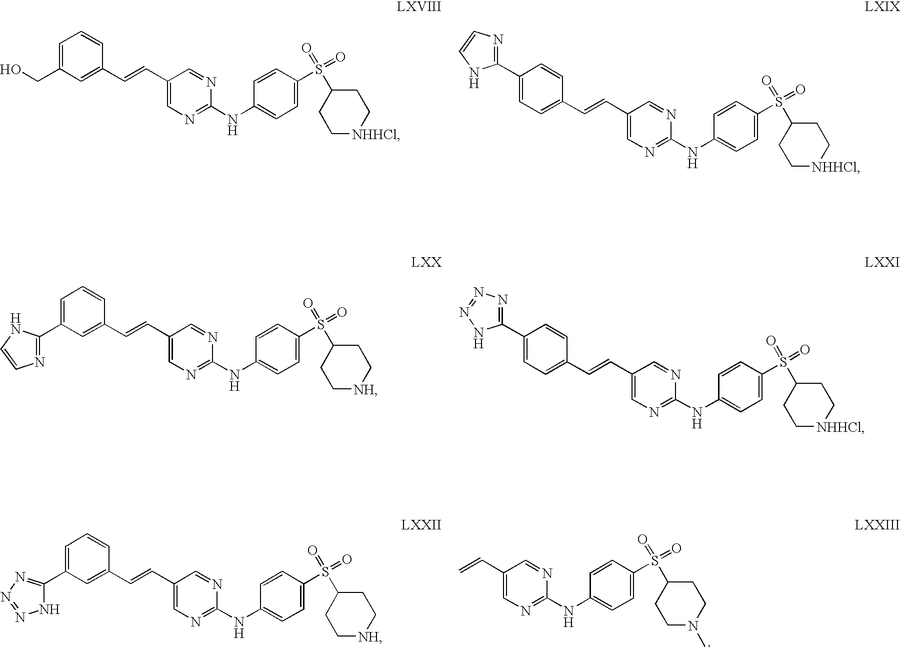

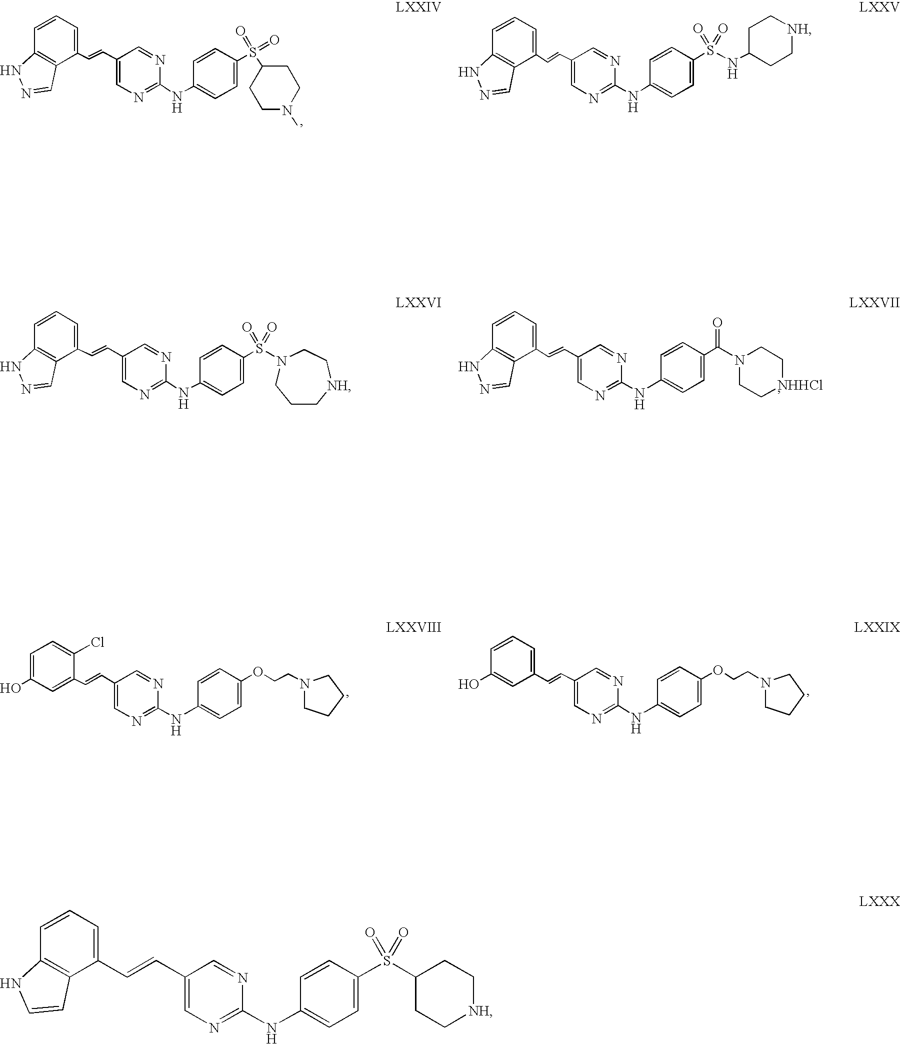

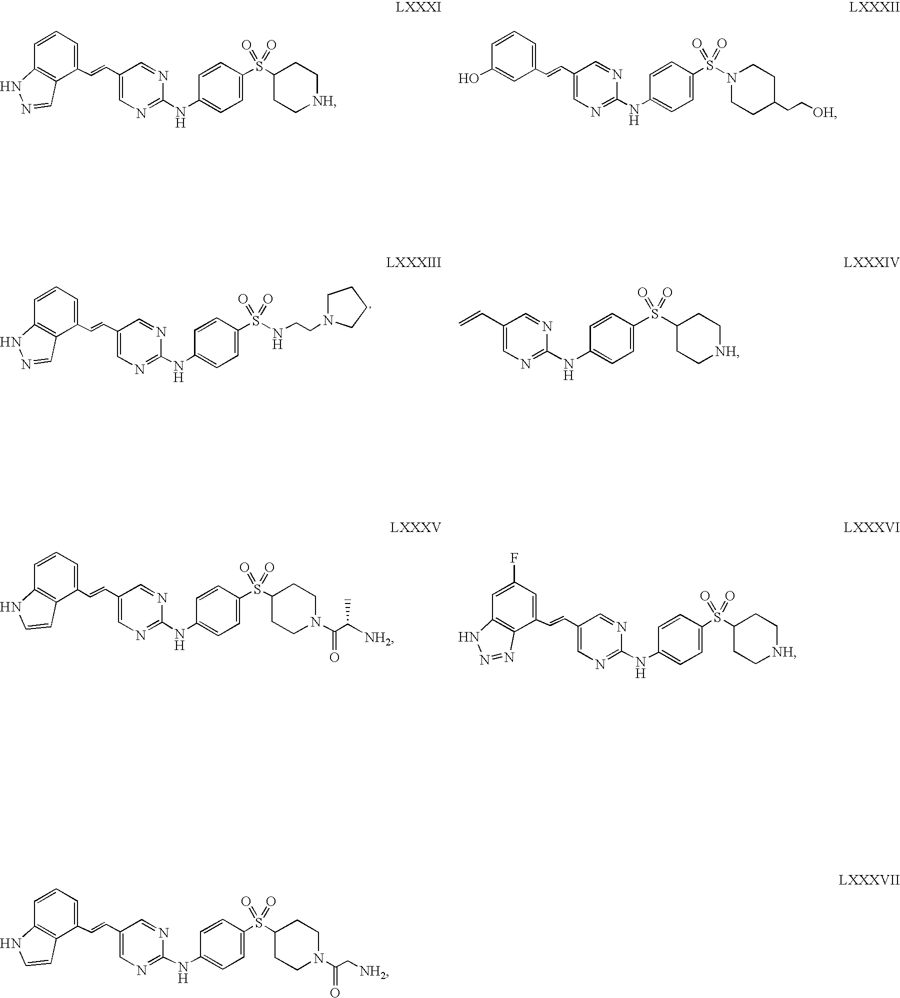

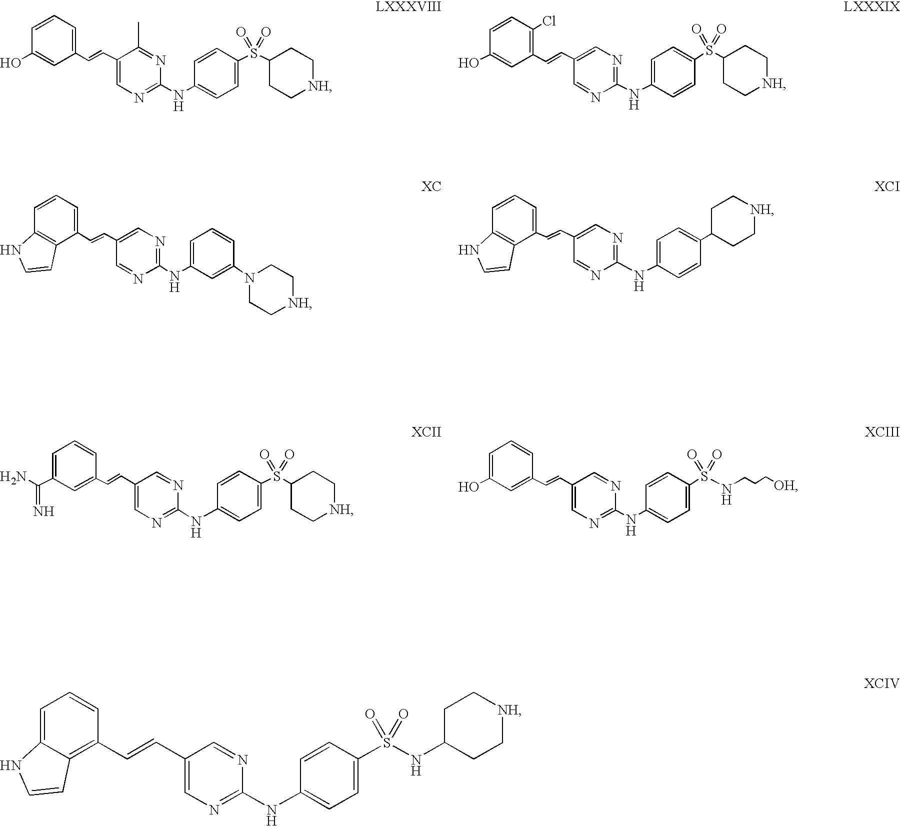

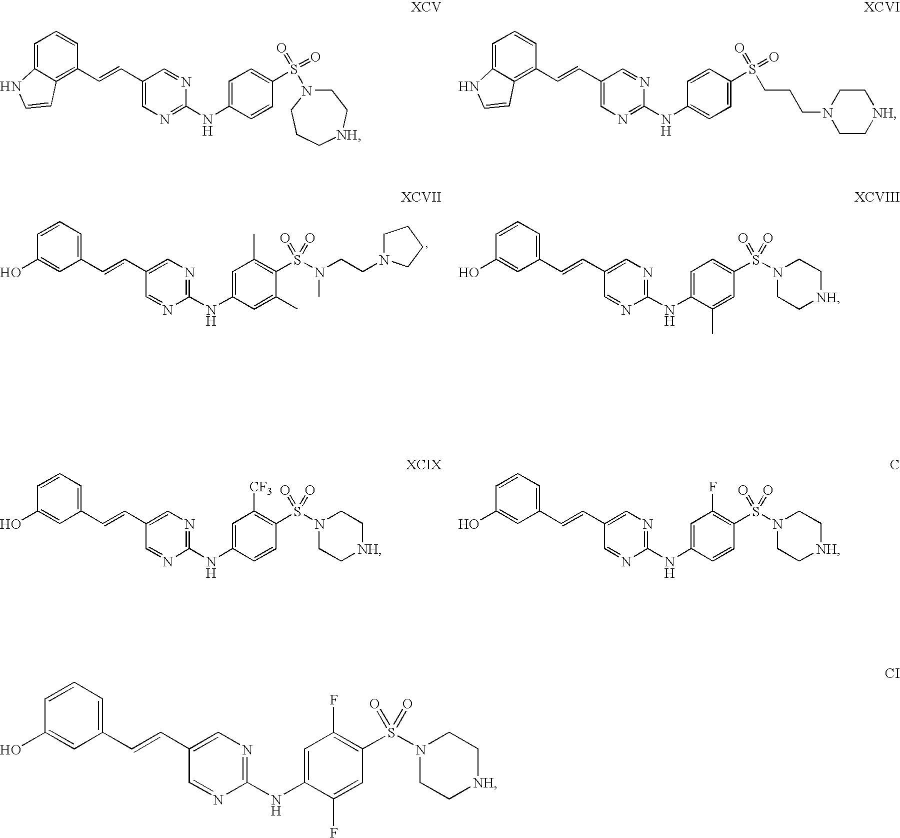

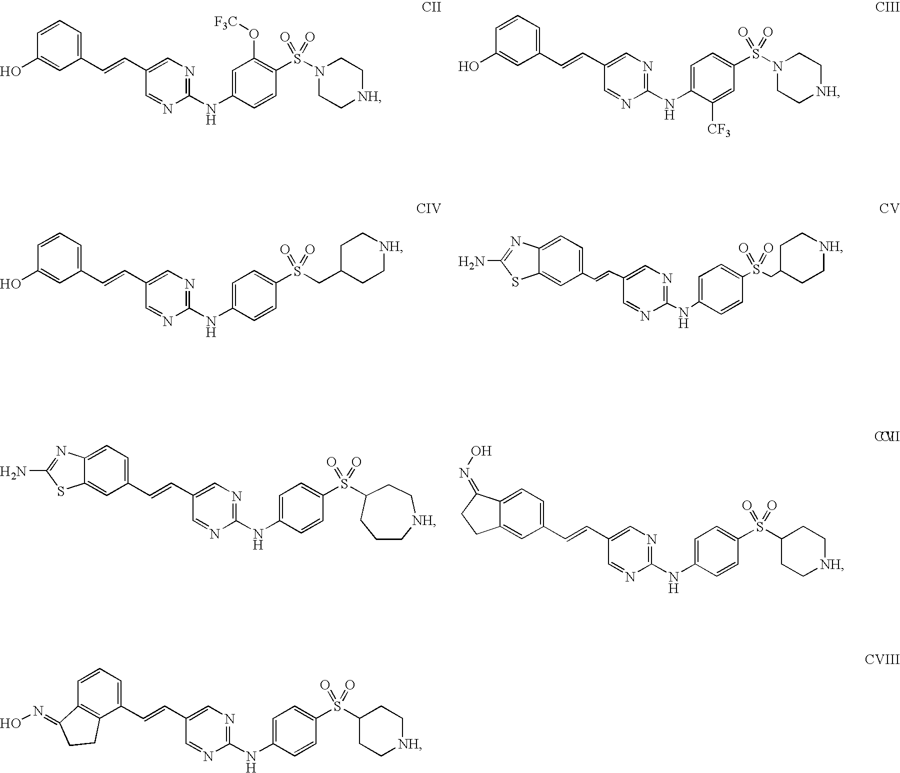

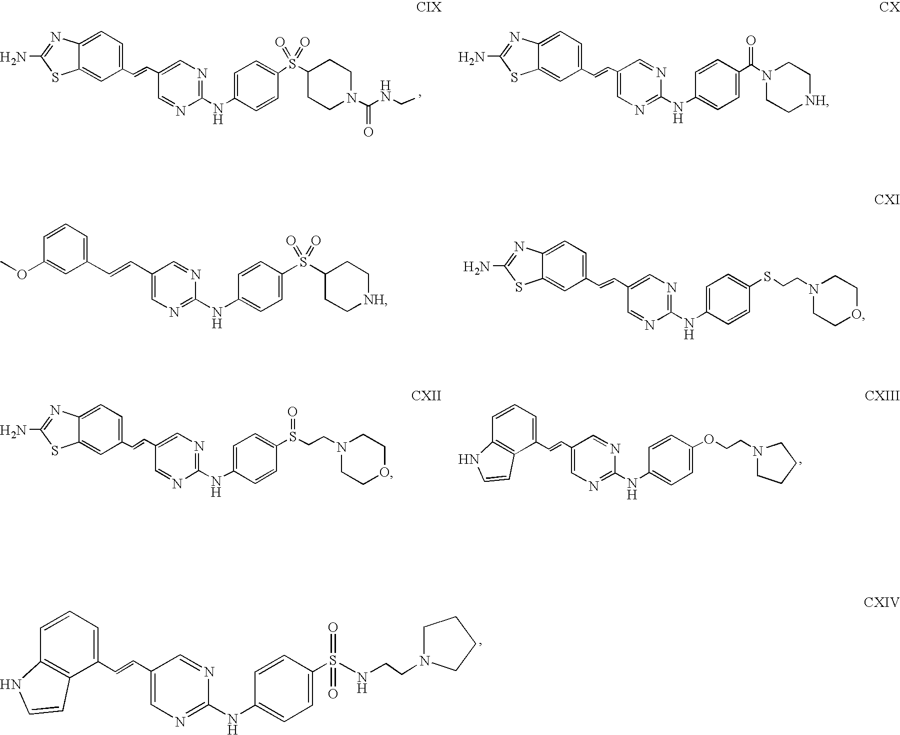

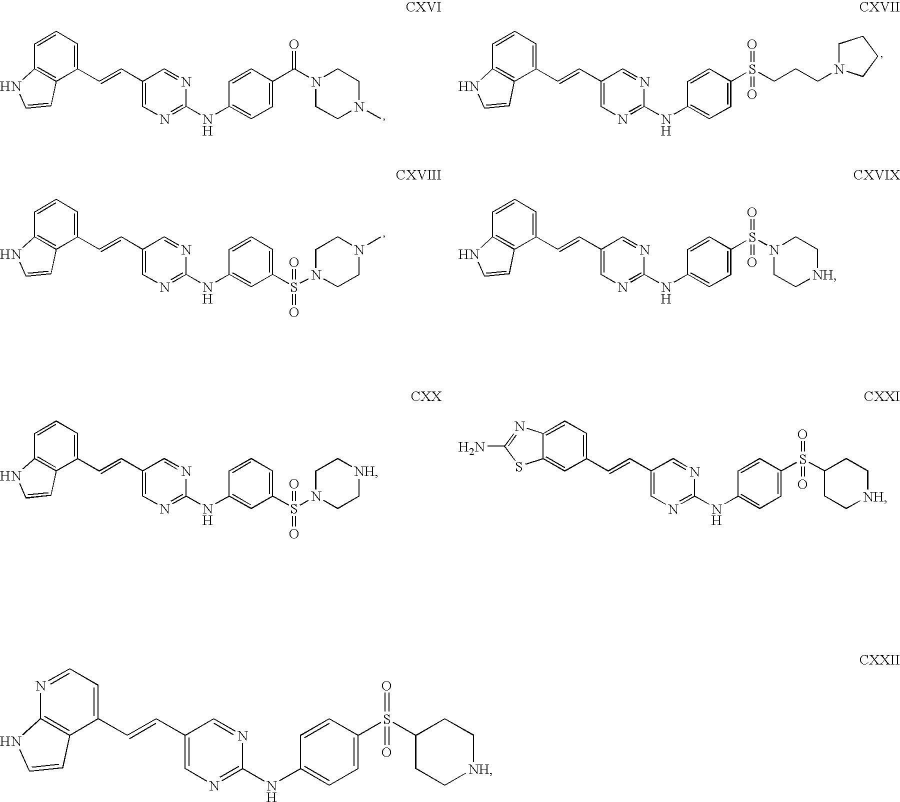

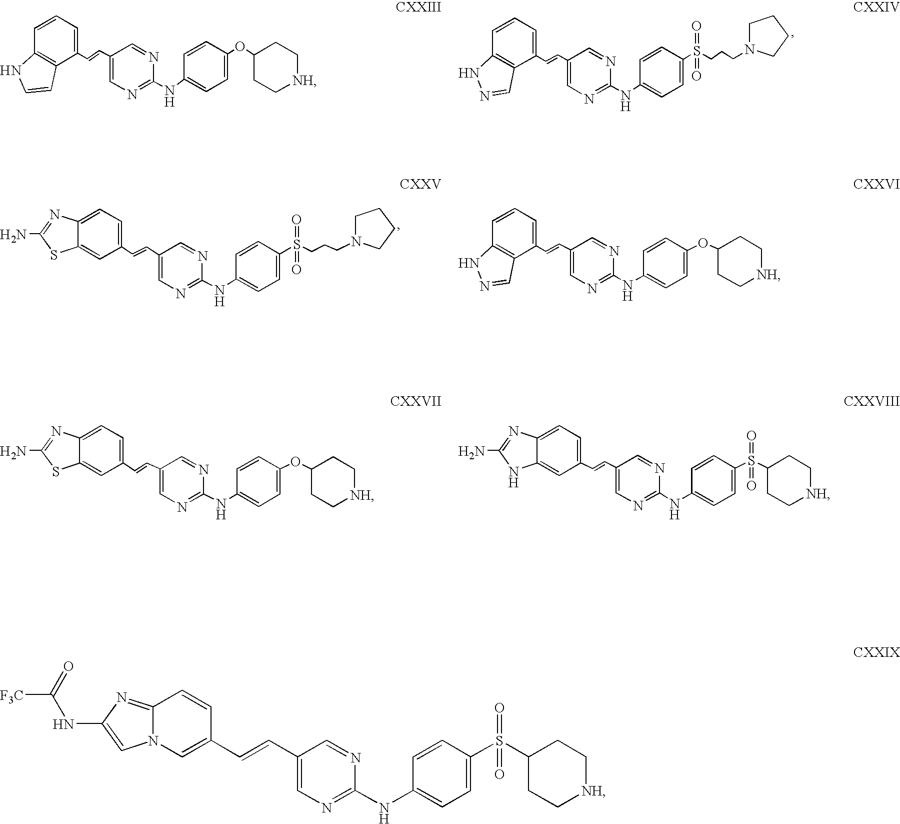

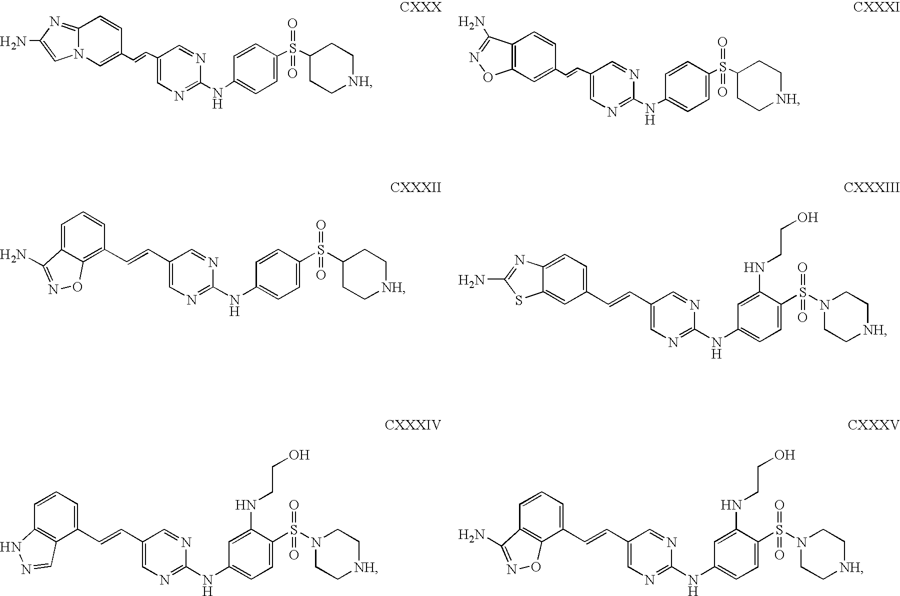

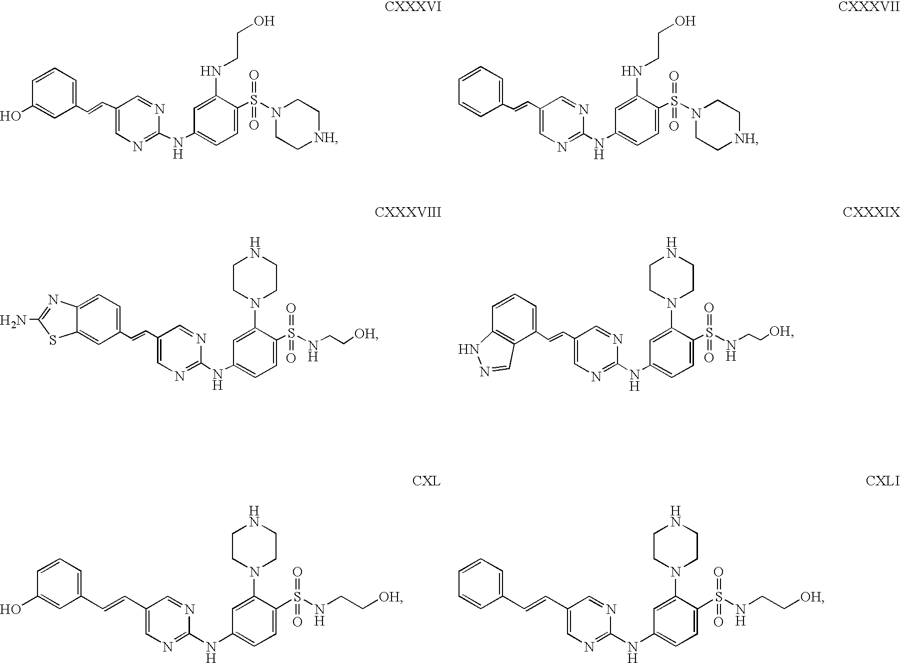

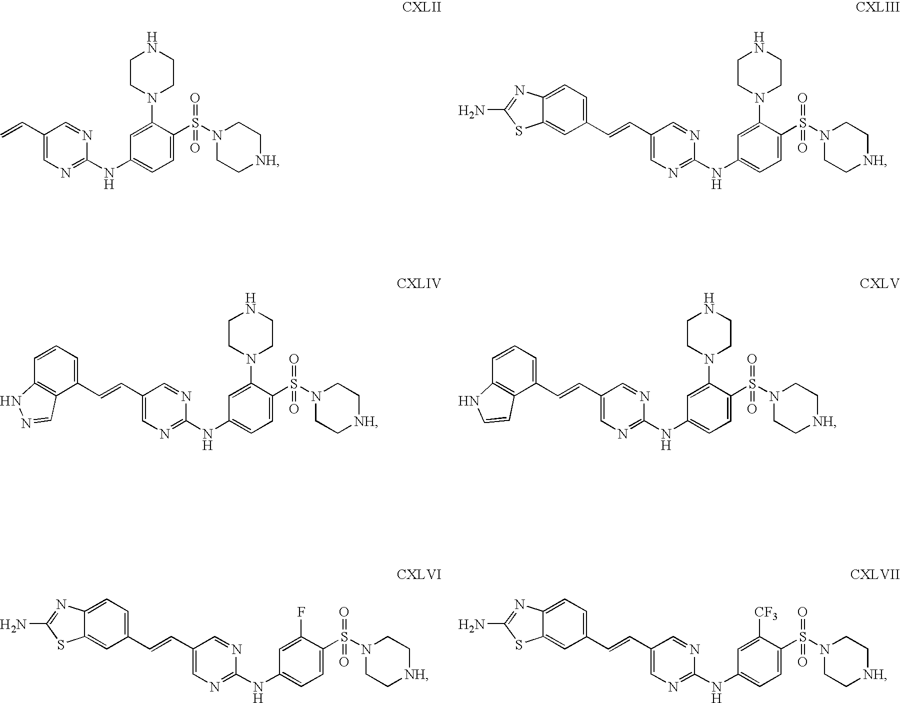

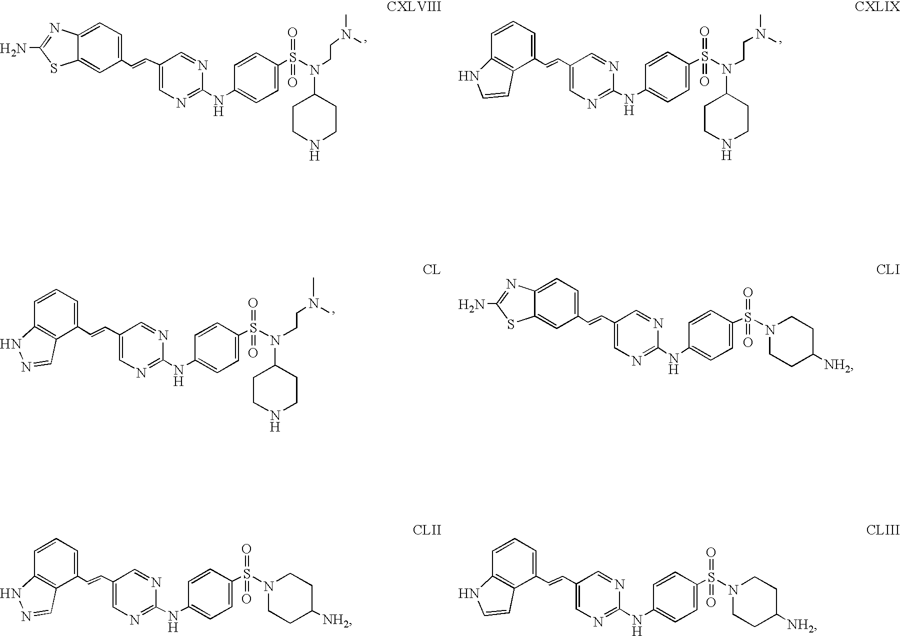

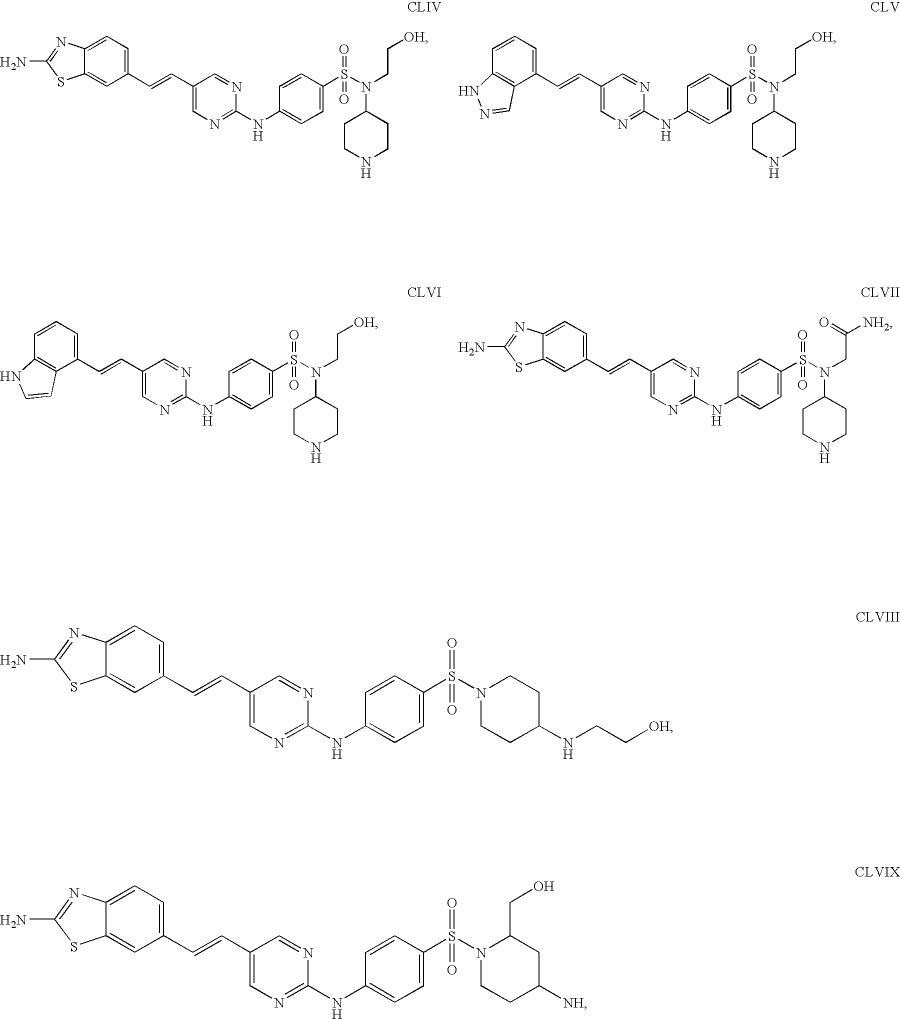

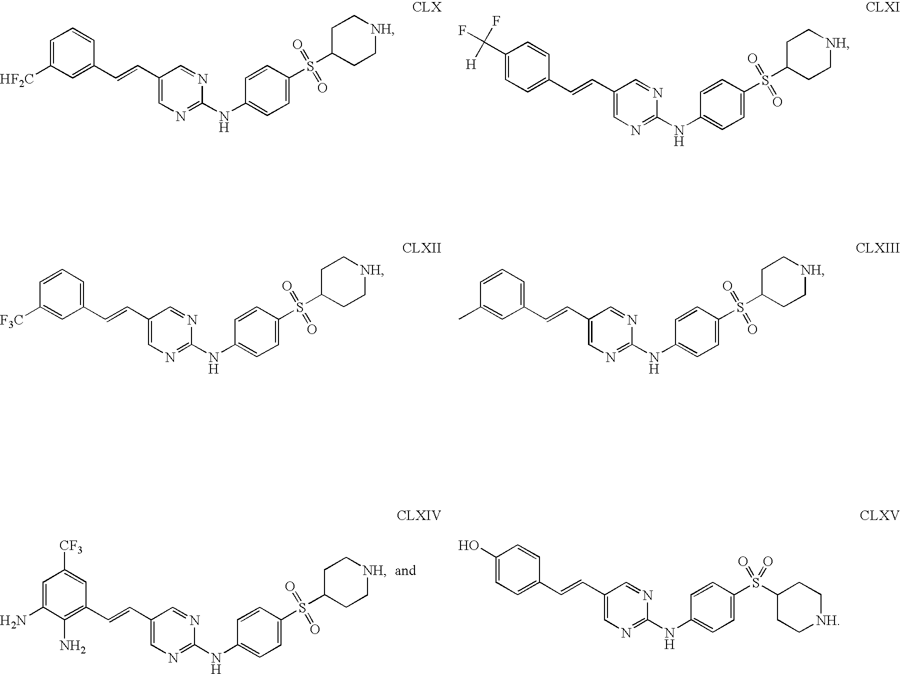

4. The compound of claim 1 or 2, wherein the compound is selected from the group consisting of:

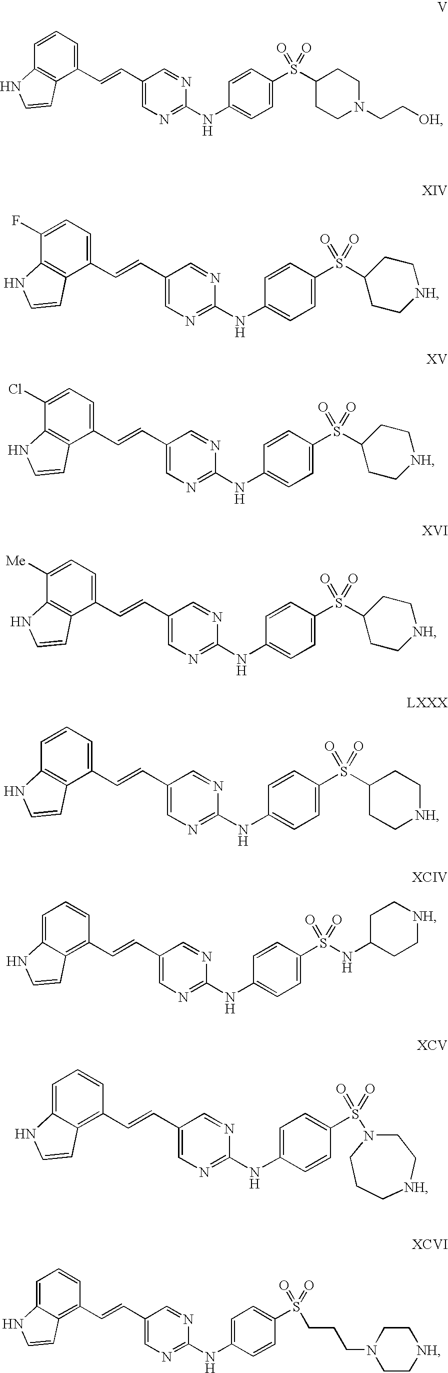

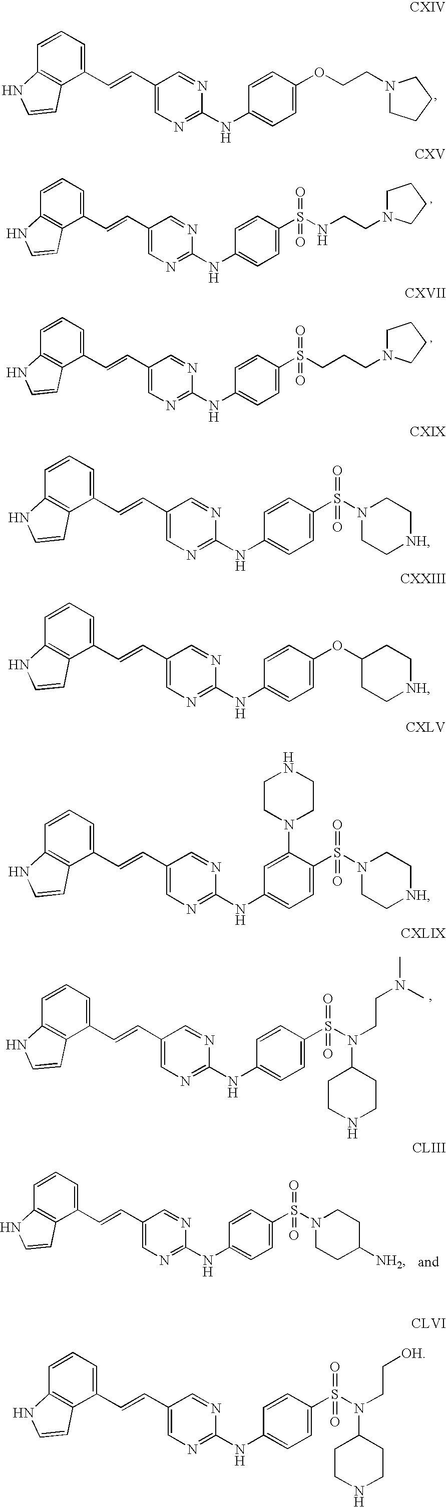

5. The compound of claim 1 or 2, wherein the compound is:

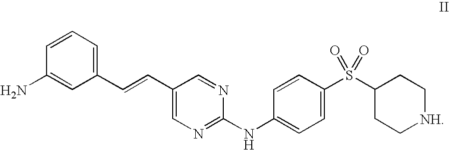

6. The compound of claim 1 or 2, wherein the compound is:

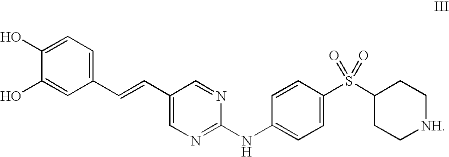

7. The compound of claim 1 or 2, wherein the compound is selected from the group consisting of:

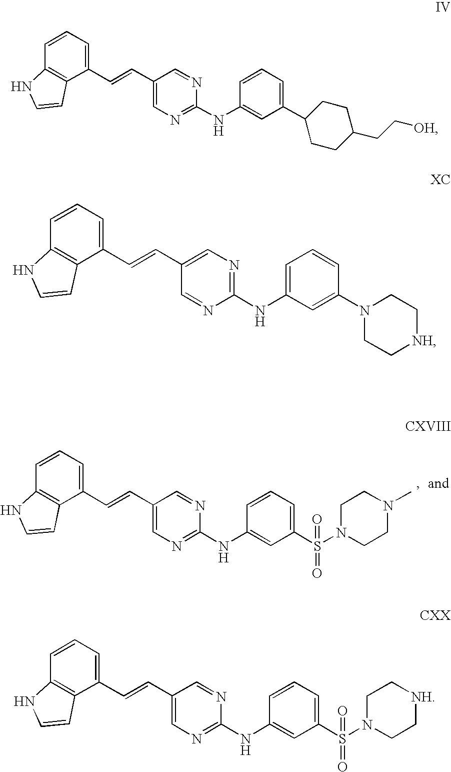

8. The compound of claim 1 or 2, wherein the compound is:

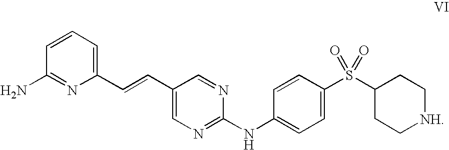

9. The compound of claim 1 or 2, wherein the compound is selected from the group consisting of:

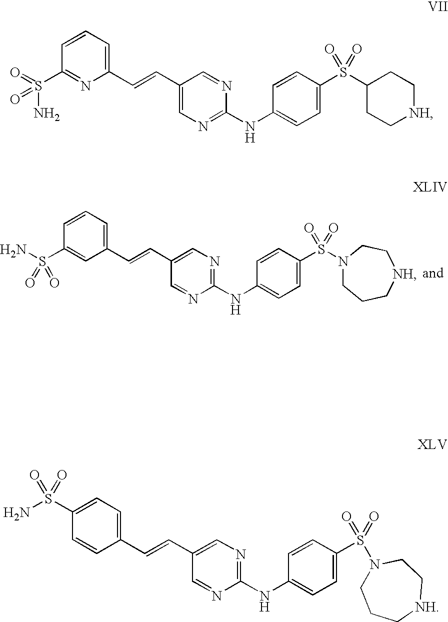

10. The compound of claim 1 or 2, wherein the compound is selected from the group consisting of:

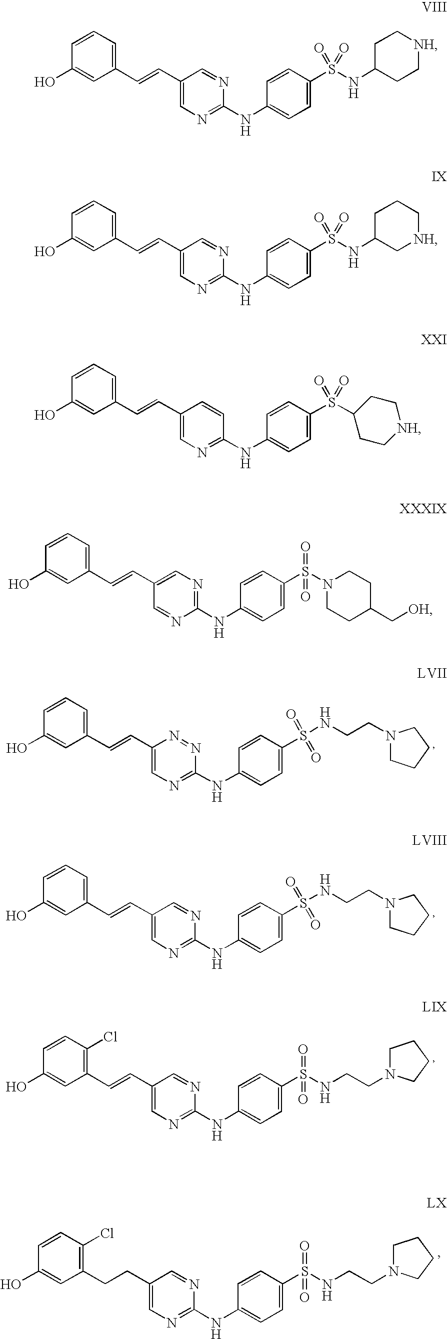

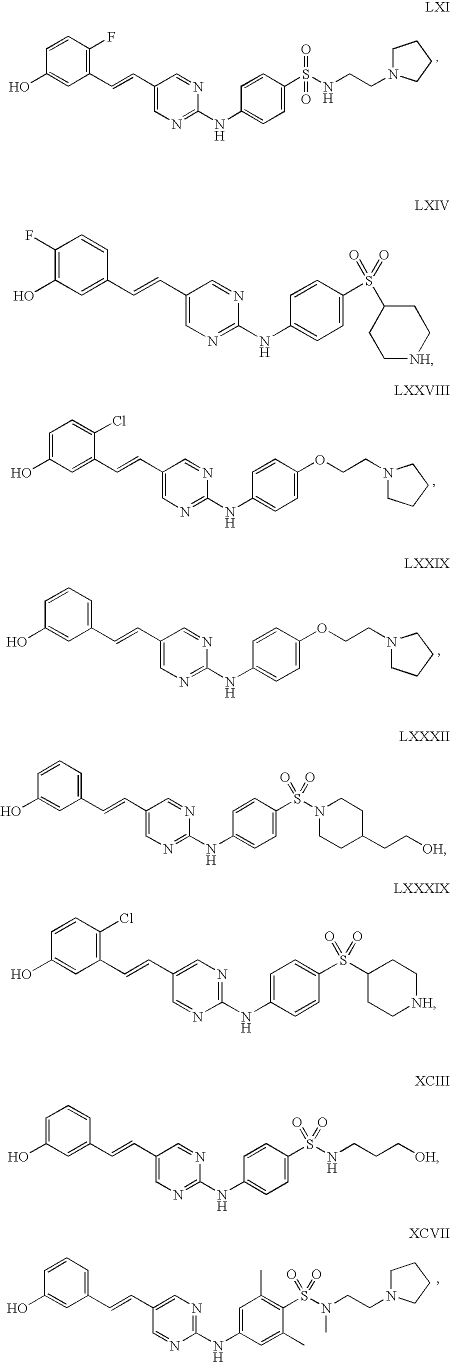

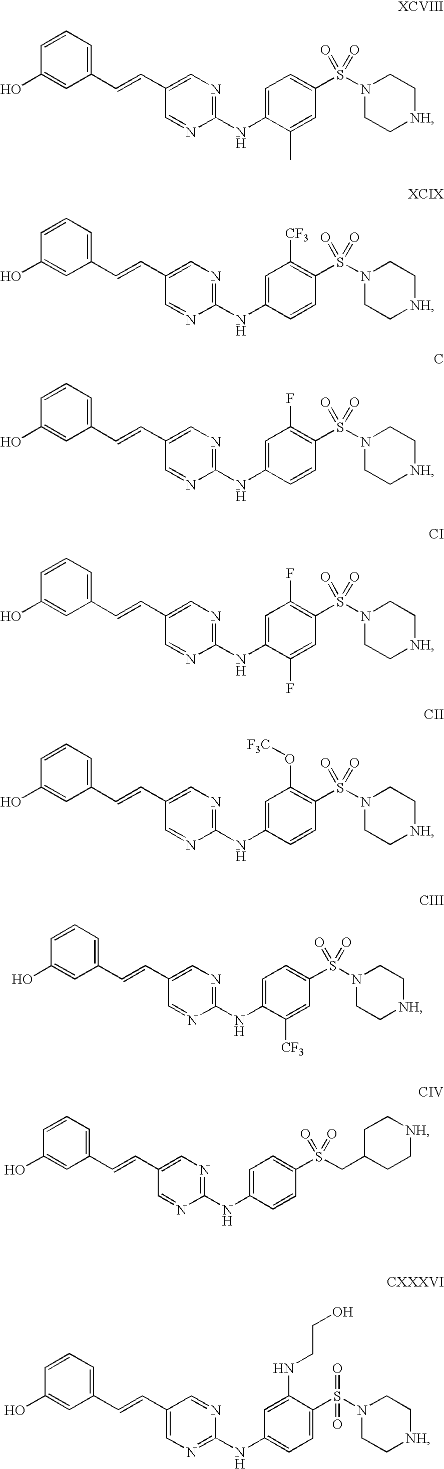

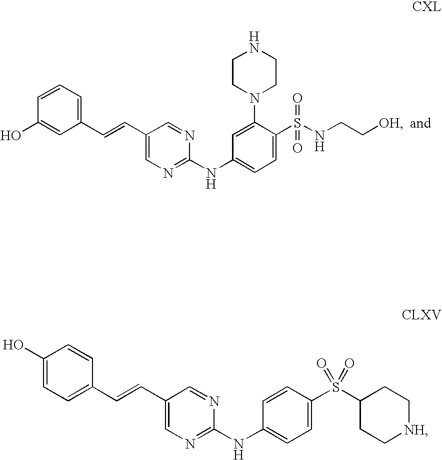

11. The compound of claim 1 or 2, wherein the compound is selected from the group consisting of:

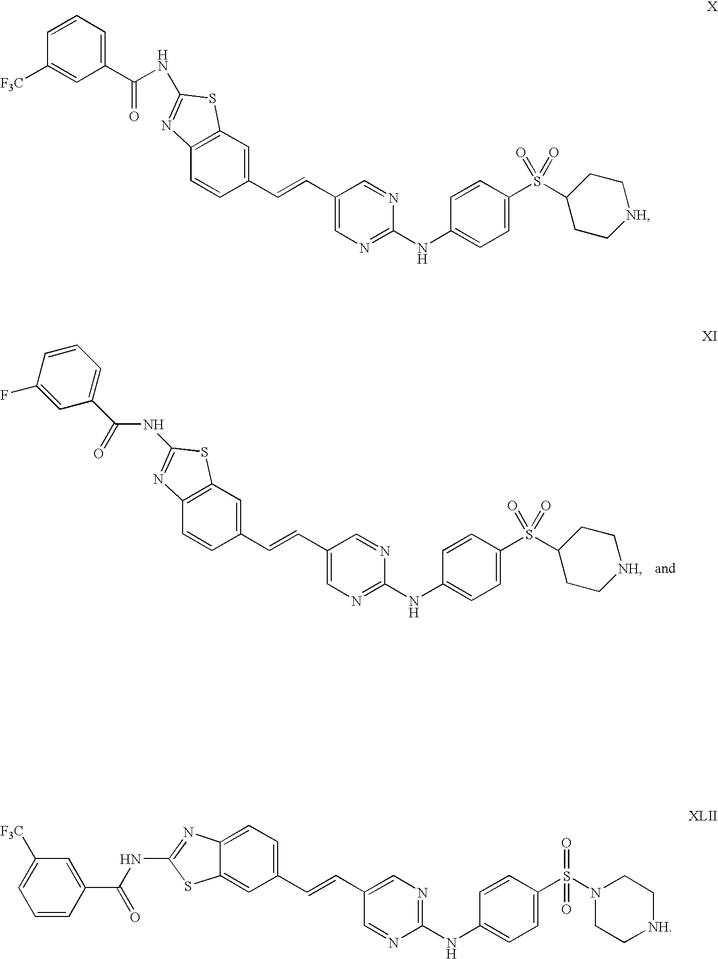

12. The compound of claim 1 or 2, wherein the compound is selected from the group consisting of:

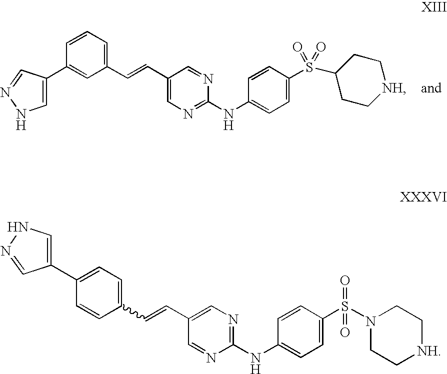

13. The compound of claim 1 or 2, wherein the compound is selected from the group consisting of:

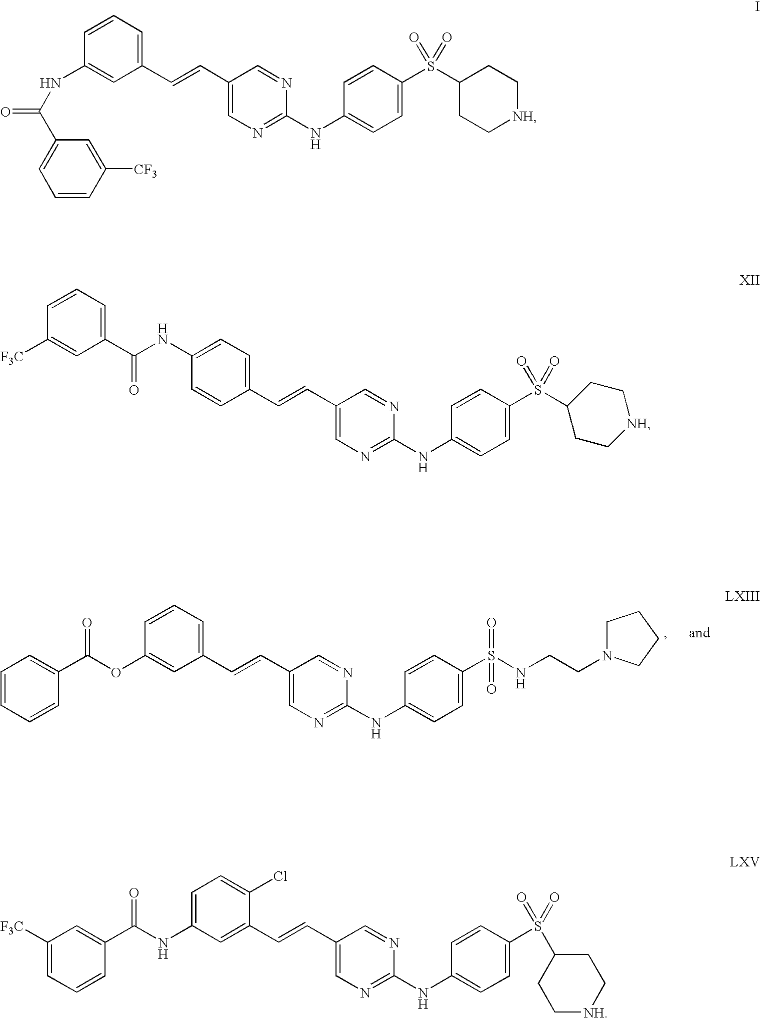

14. The compound of claim 1 or 2, wherein the compound is:

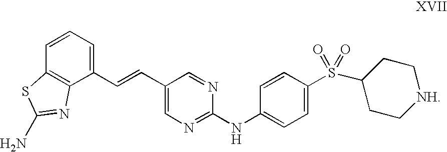

15. The compound of claim 1 or 2, wherein the compound is:

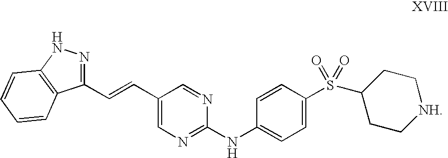

16. The compound of claim 1 or 2, wherein the compound is selected from the group consisting of:

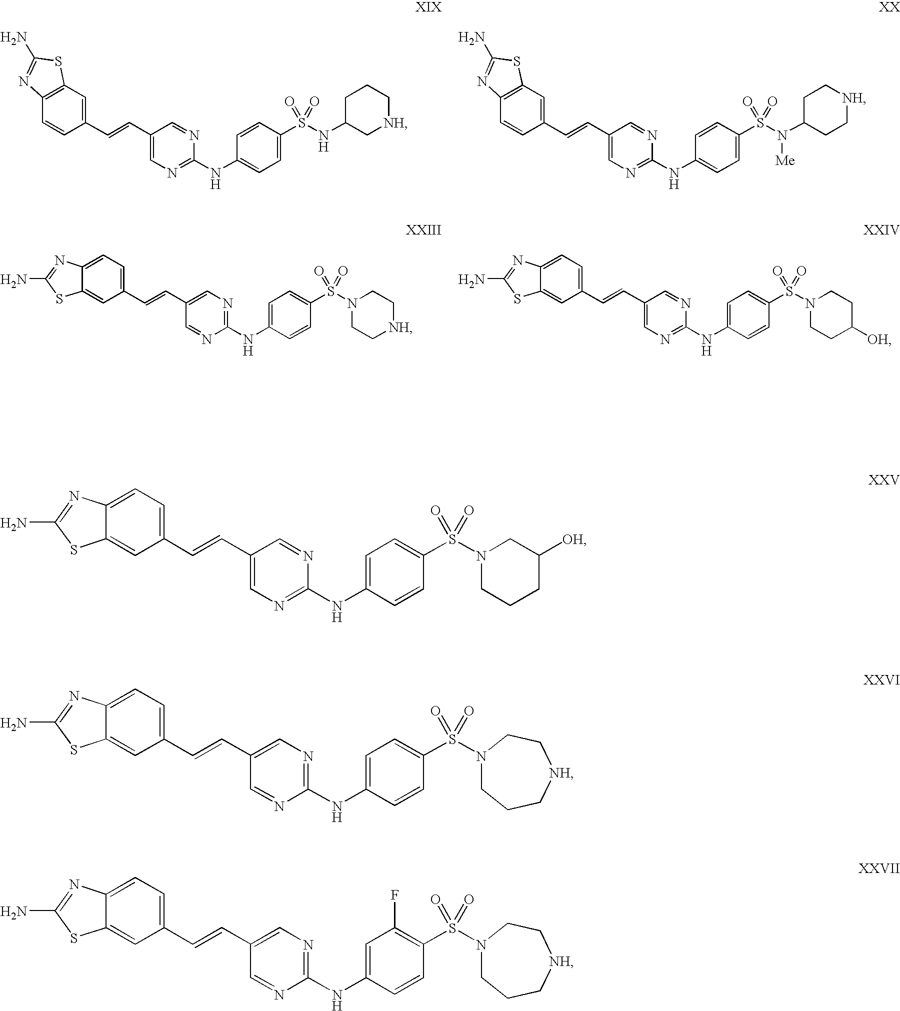

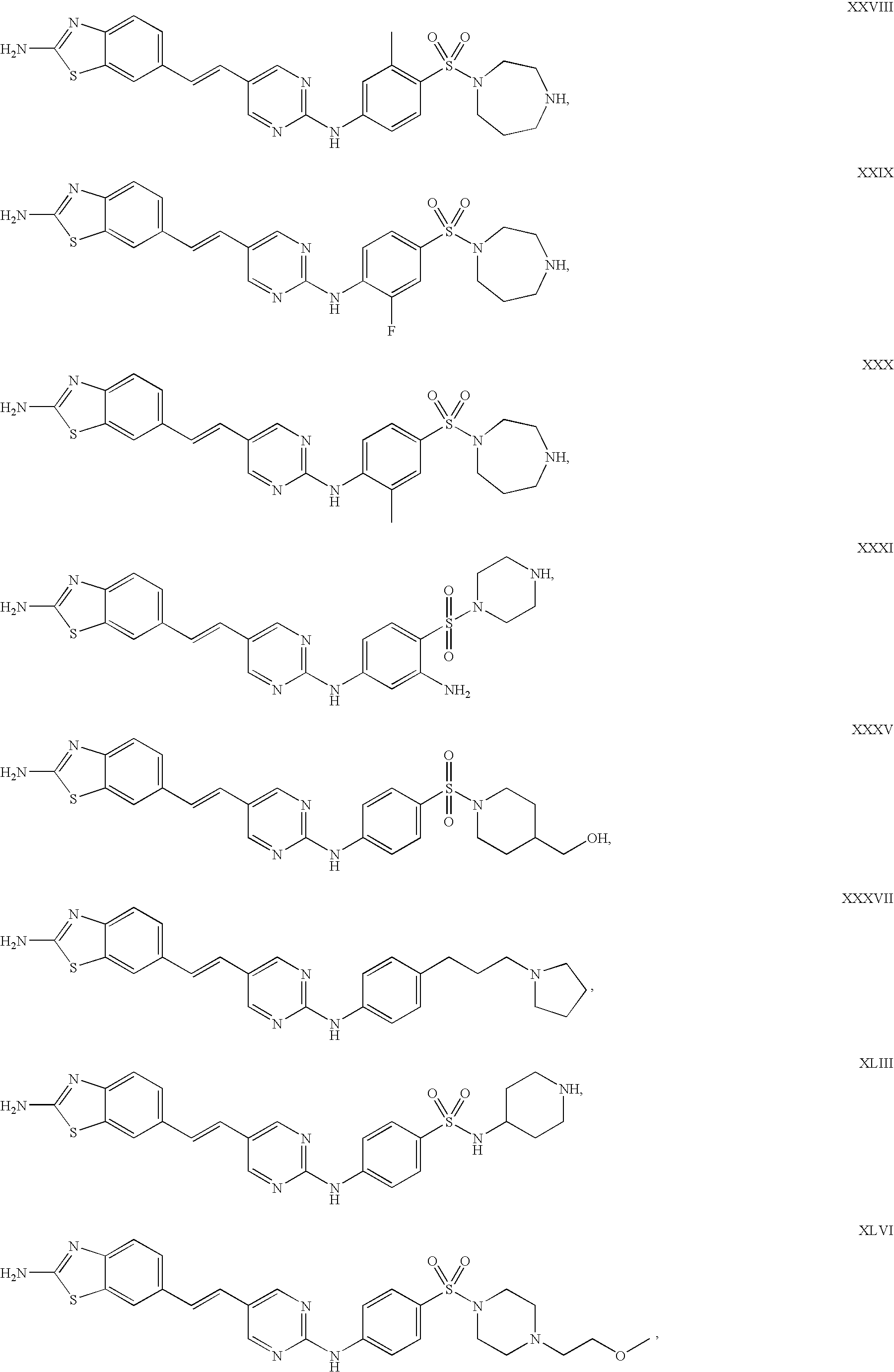

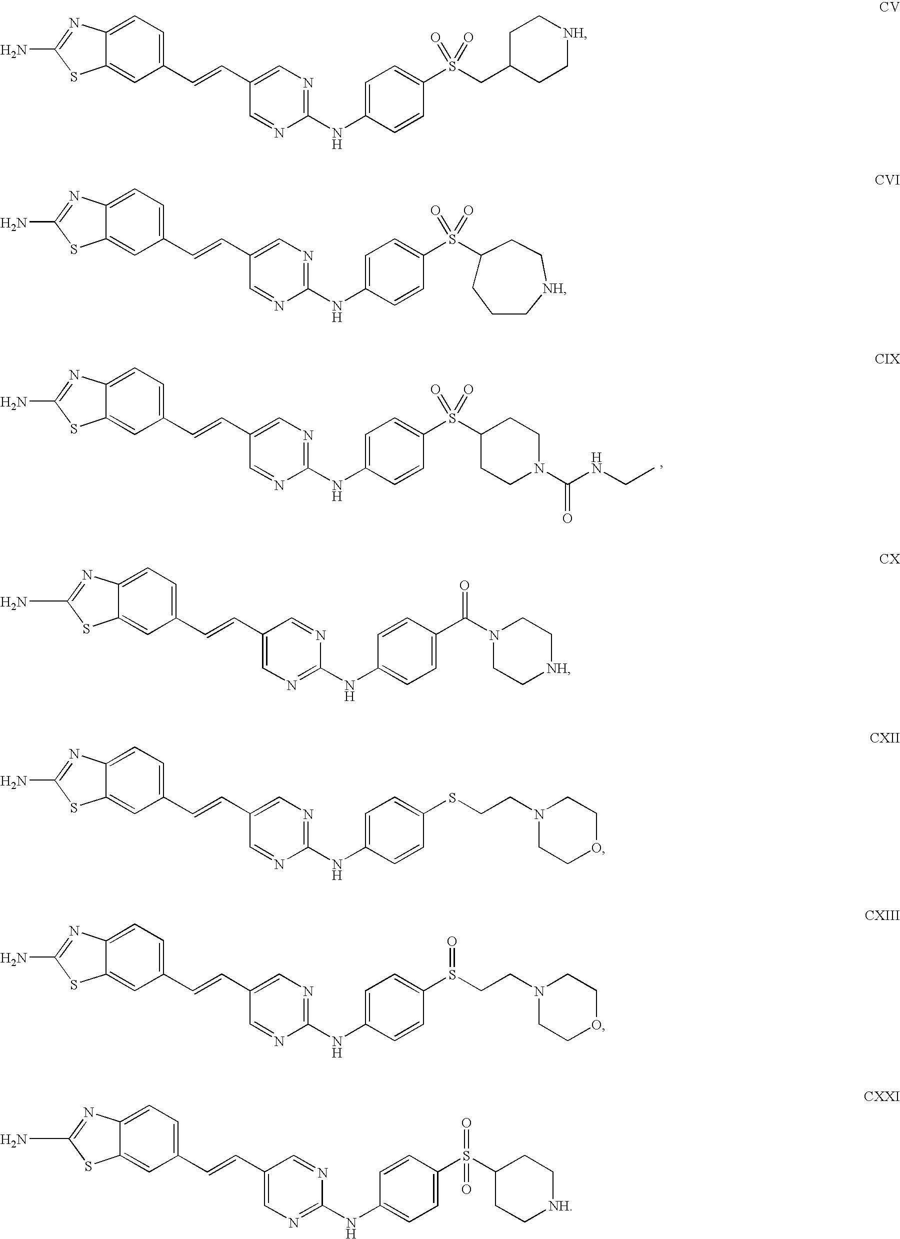

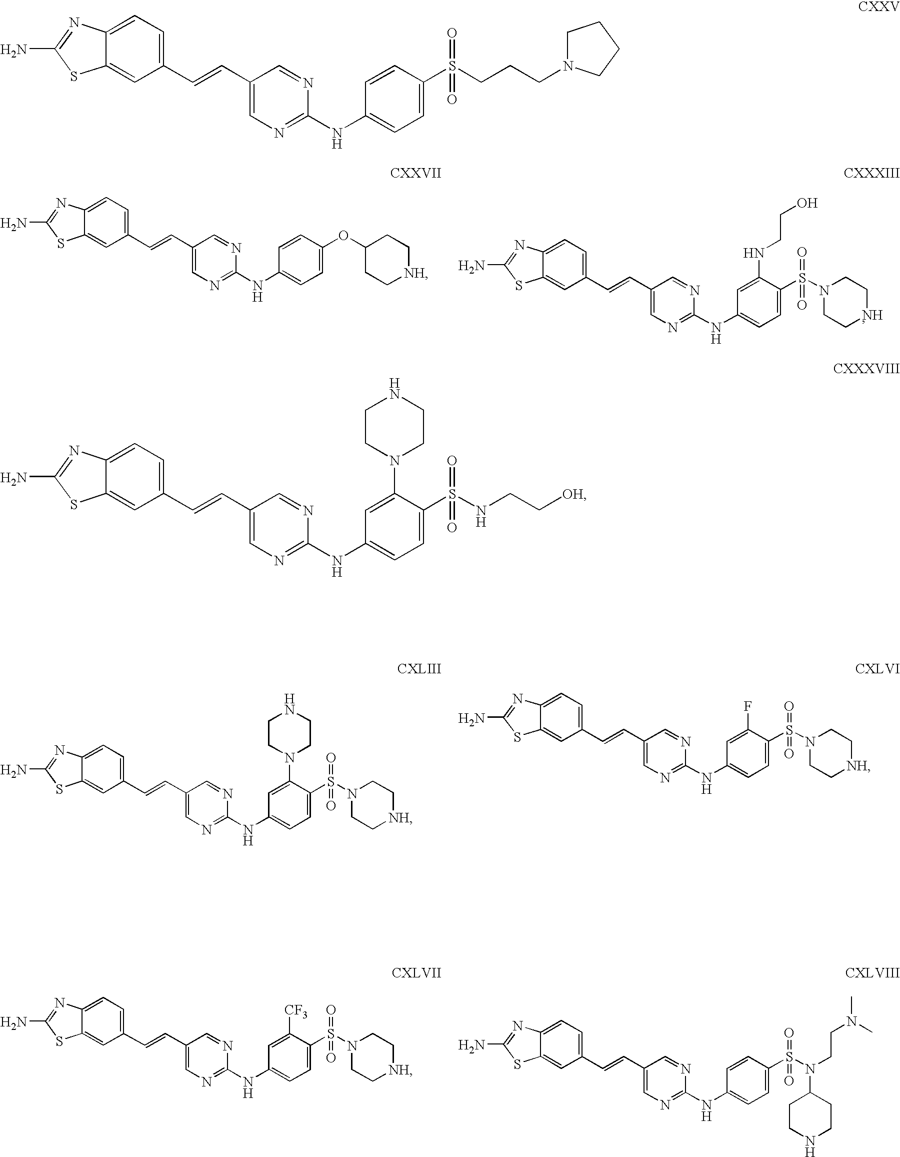

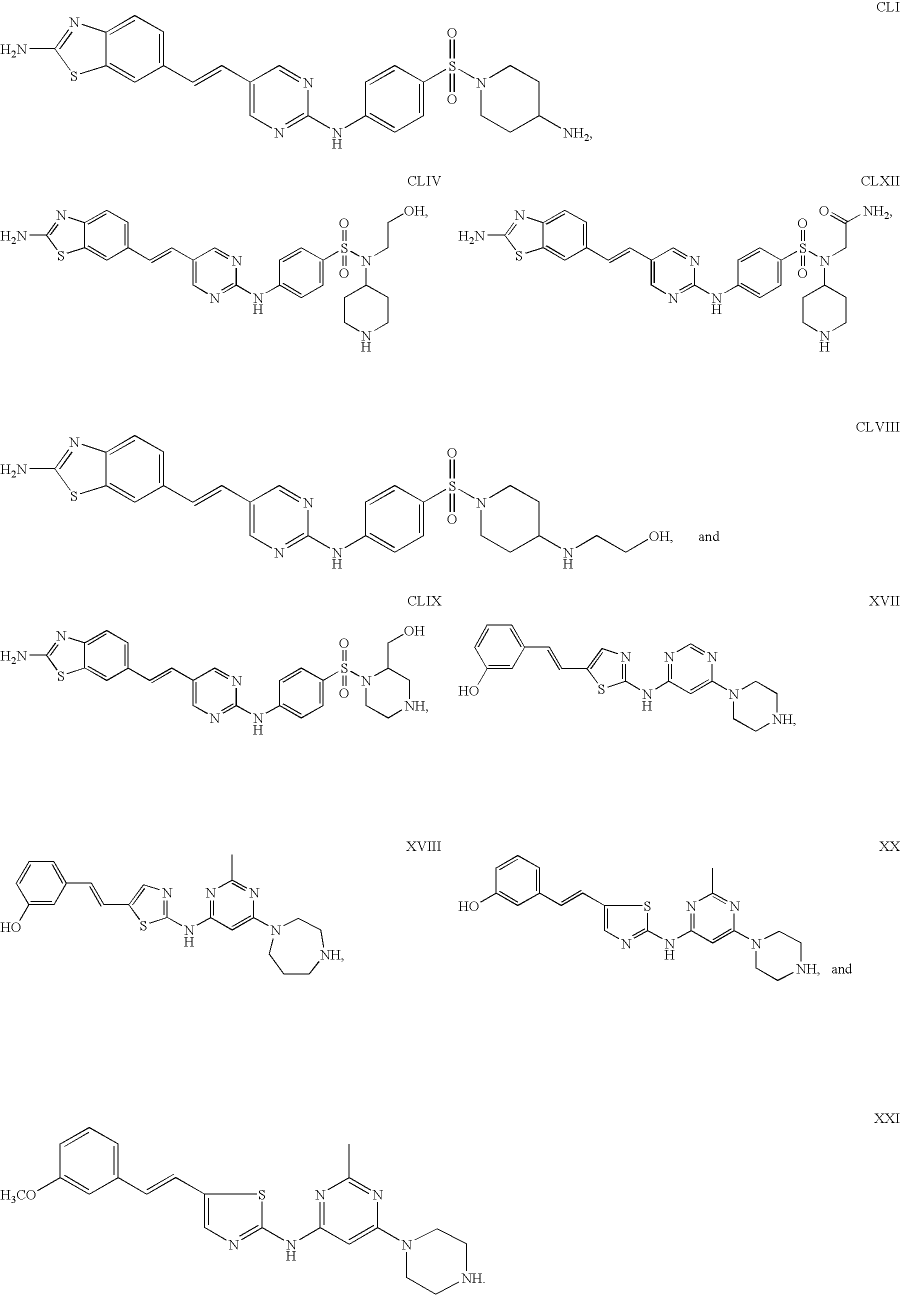

17. The compound of claim 1 or 2, wherein the compound is selected from the group consisting of:

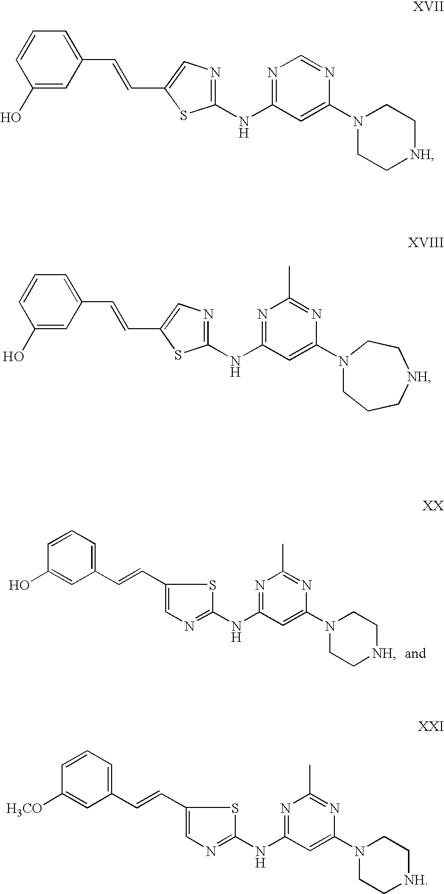

18. The compound of claim 1 or 2, wherein the compound is selected from the group consisting of:

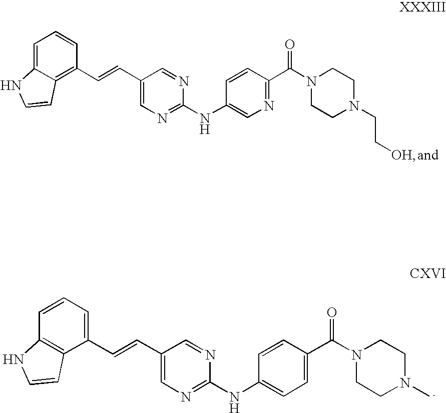

19. The compound of claim 1 or 2, wherein the compound is:

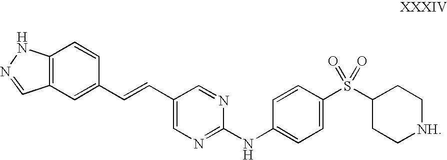

20. The compound of claim 1 or 2, wherein the compound is:

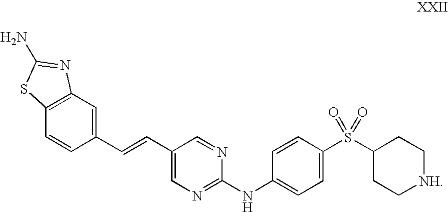

21. The compound of claim 1 or 2, wherein the compound is:

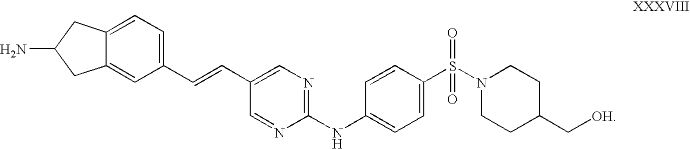

22. The compound of claim 1 or 2, wherein the compound is selected from the group consisting of:

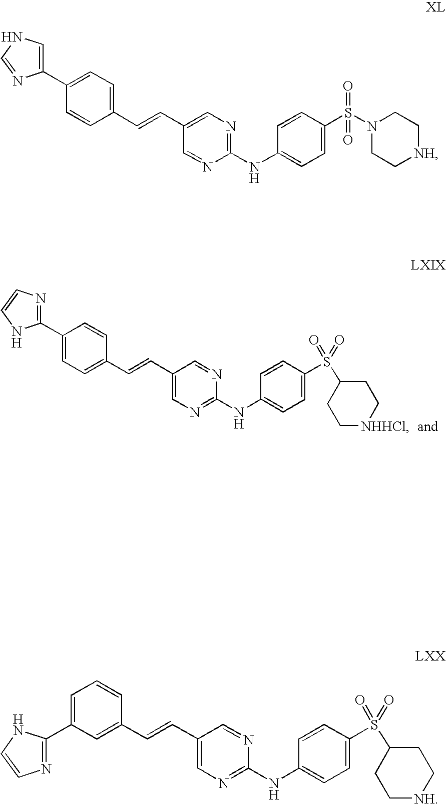

23. The compound of claim 1 or 2, wherein the compound is selected from the group consisting of:

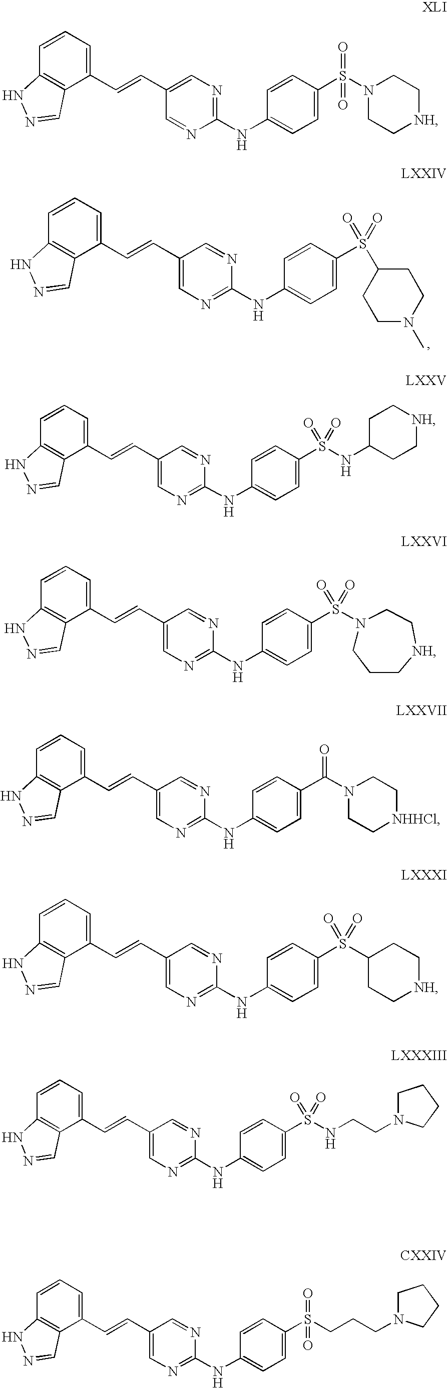

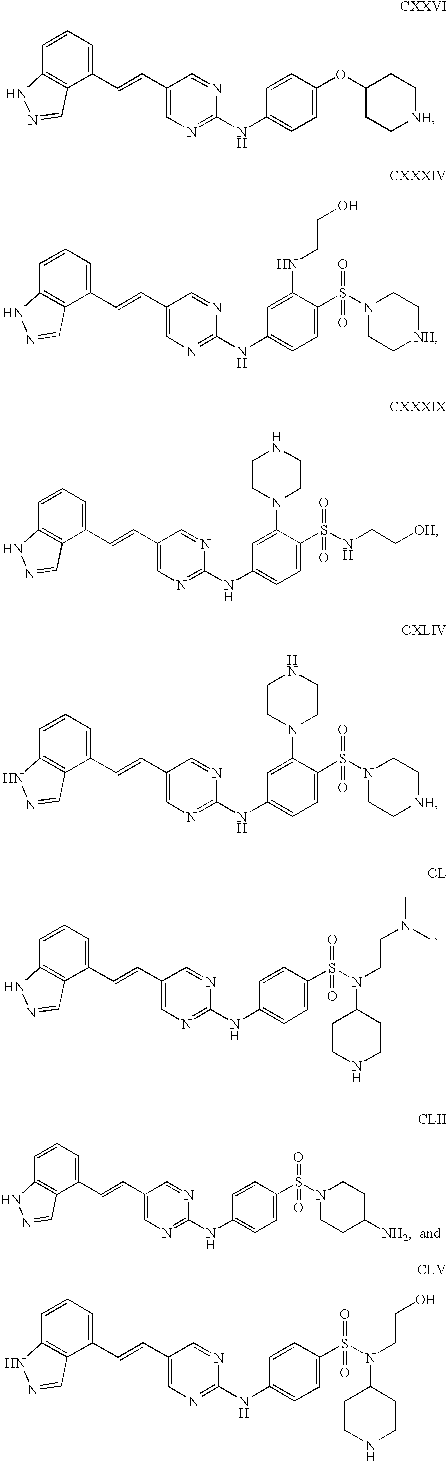

24. The compound of claim 1 or 2, wherein the compound is selected from the group consisting of:

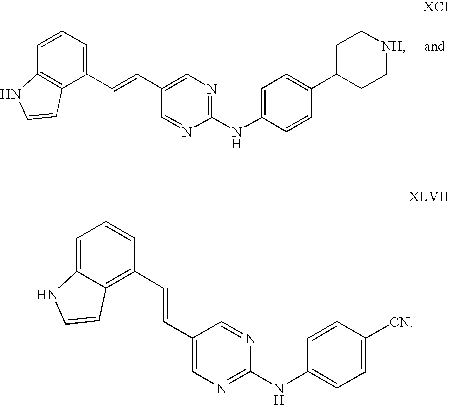

25. The compound of claim 1 or 2, wherein the compound is selected from the group consisting of:

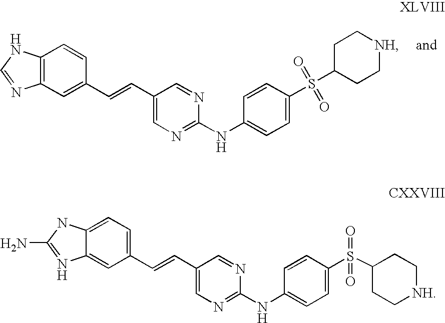

26. The compound of claim 1 or 2, wherein the compound is selected from the group consisting of:

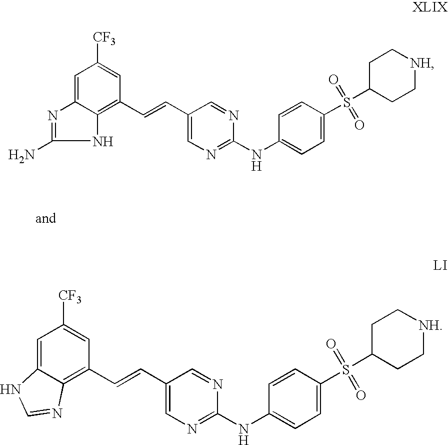

27. The compound of claim 1 or 2, wherein the compound is selected from the group consisting of:

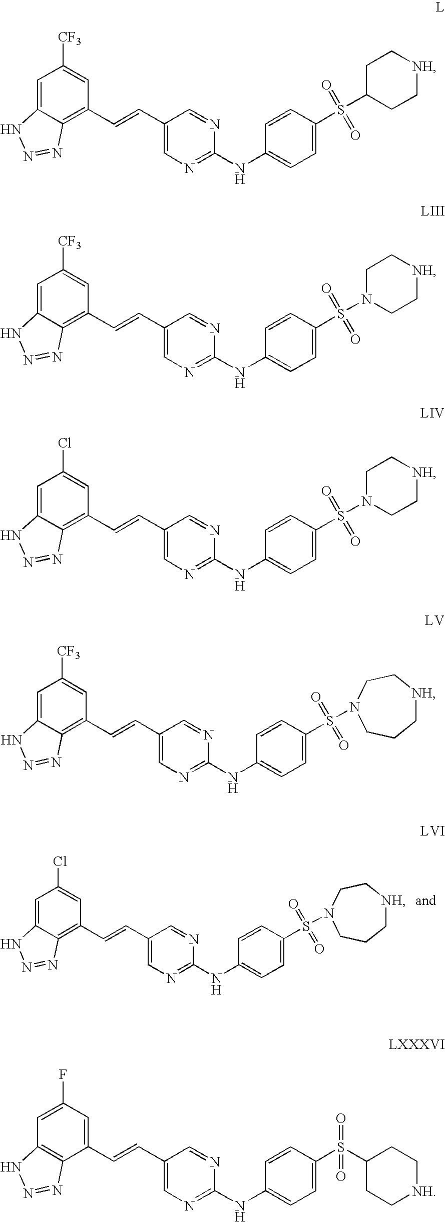

28. The compound of claim 1 or 2, wherein the compound is:

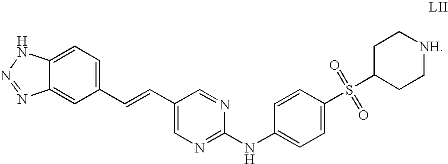

29. The compound of claim 1 or 2, wherein the compound is selected from the group consisting of:

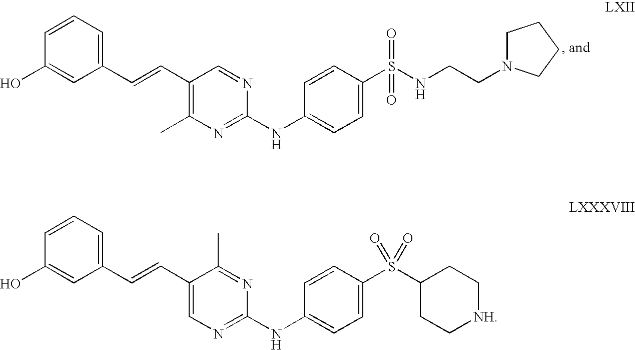

30. The compound of claim 1 or 2, wherein the compound is:

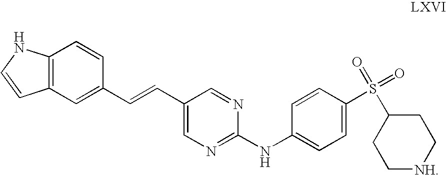

31. The compound of claim 1 or 2, wherein the compound is:

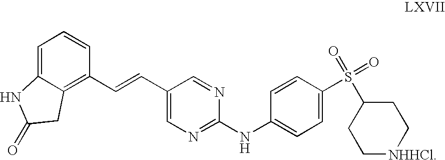

32. The compound of claim 1 or 2, wherein the compound is:

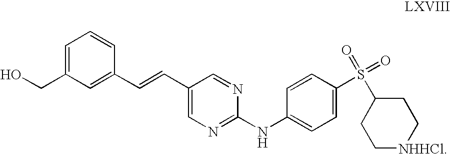

33. The compound of claim 1 or 2, wherein the compound is selected from the group consisting of:

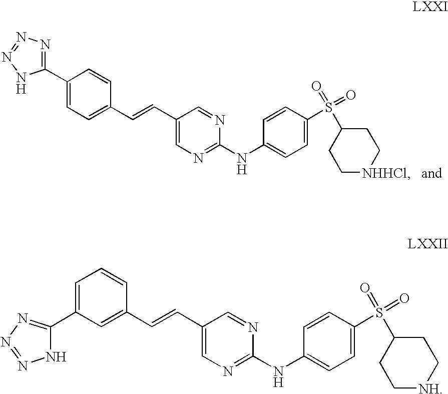

34. The compound of claim 1 or 2, wherein the compound is selected from the group consisting of:

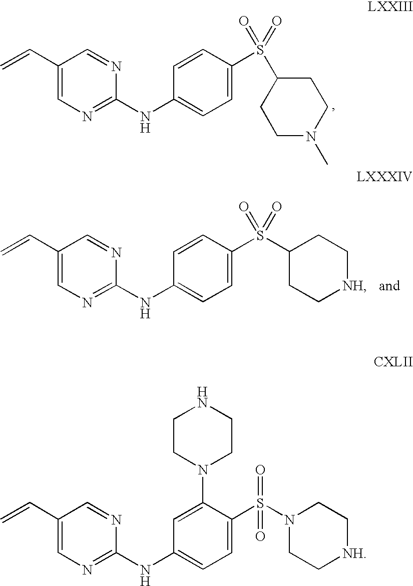

35. The compound of claim 1 or 2, wherein the compound is selected from the group consisting of:

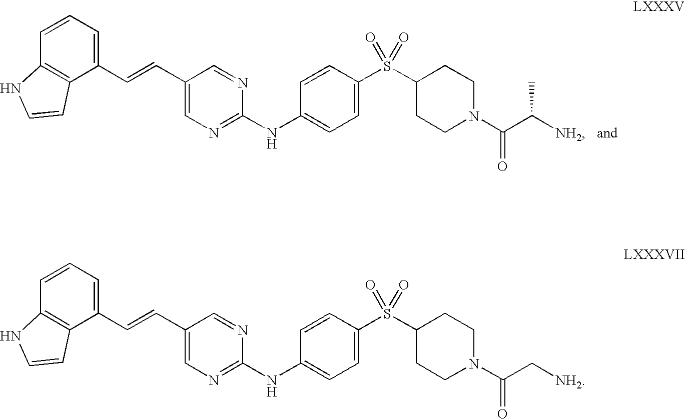

36. The compound of claim 1 or 2, wherein the compound is:

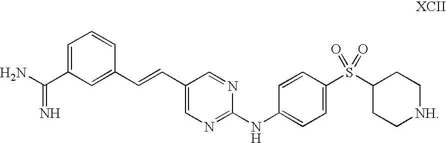

37. The compound of claim 1 or 2, wherein the compound is:

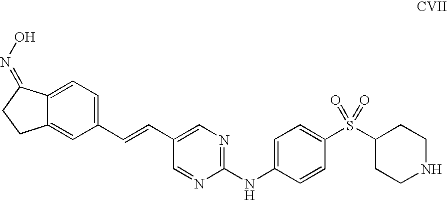

38. The compound of claim 1 or 2, wherein the compound is:

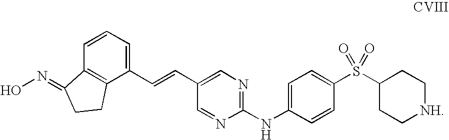

39. The compound of claim 1 or 2, wherein the compound is:

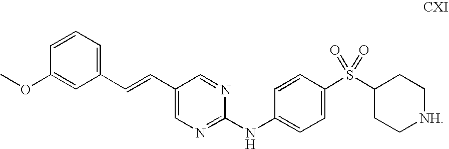

40. The compound of claim 1 or 2, wherein the compound is:

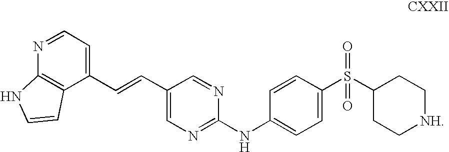

41. The compound of claim 1 or 2, wherein the compound is selected from the group consisting of:

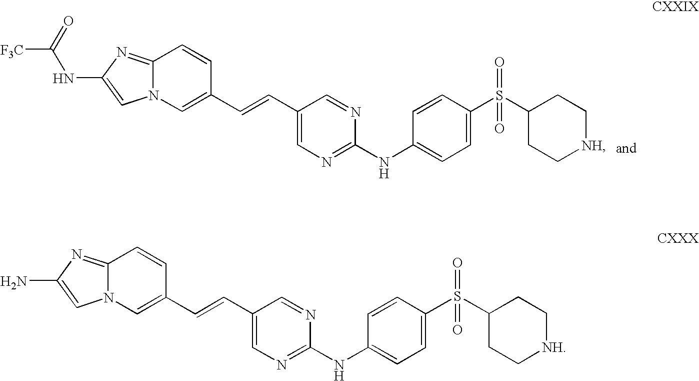

42. The compound of claim 1 or 2, wherein the compound is:

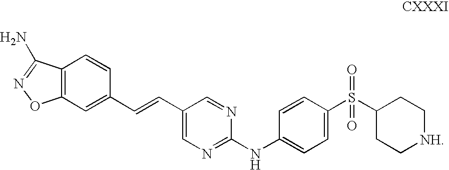

43. The compound of claim 1 or 2, wherein the compound is selected from the group consisting of:

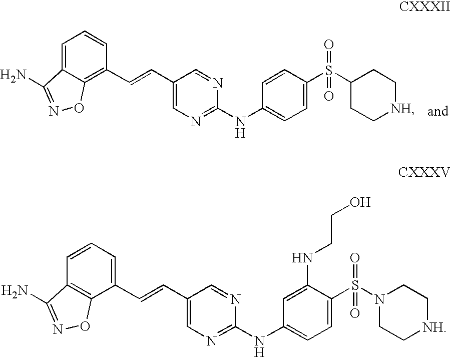

44. The compound of claim 1 or 2, wherein the compound is selected from the group consisting of:

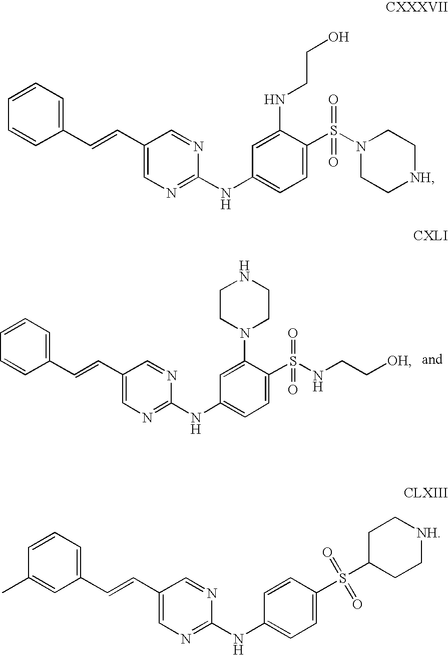

45. The compound of claim 1 or 2, wherein the compound is selected from the group consisting of:

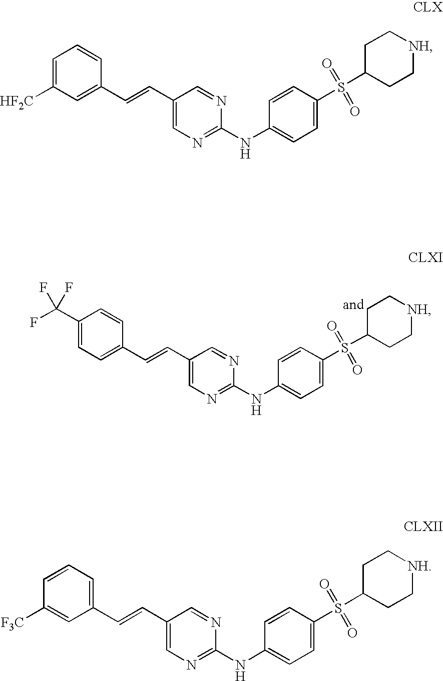

46. The compound of claim 1 or 2, wherein the compound is:

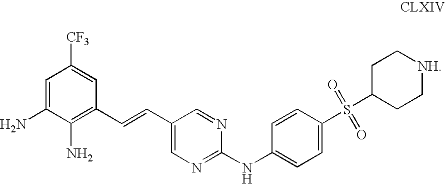

47. A method for treating a disorder, comprising:
(a) in a population of patients in need of the treatment, determining a group of patients who do not respond to any therapy, or any combination of a plurality of therapies, wherein said therapy or therapies comprise administering currently used medications;
(b) administering to a member of the non-responding population a therapeutically effective amount of at least one compound of claim 1 or 2, or pharmaceutically acceptable N-oxide(s), salts, hydrates, solvates, crystal forms and individual diastereomers thereof.

48. The method of claim 47, wherein the currently used medication includes a compound (B), a compound (C), or a compound (D):

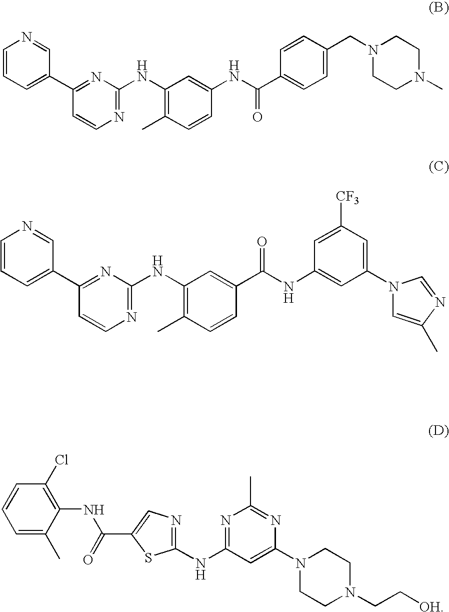

49. The method of claim 47, wherein the non-responsiveness to the kinase-inhibition therapy is caused by the kinase mutation.

50. The method of claim 49, wherein the kinase mutation is the gatekeeper residue mutation.

51. The method of claim 47, wherein the currently used medications comprise GLEEVEC, SPRYCEL, and TASIGNA.

52. The method of claim 51, wherein the currently used medication is GLEEVEC.

53. The method of claim 51, wherein the currently used medication is SPRYCEL.

54. The method of claim 51, wherein the currently used medication is TASIGNA.

55. The method of claim 47, wherein said therapy is a kinase inhibition therapy.

56. The method of claim 47, wherein the disorder is myeloid leukemia in any stage.

57. The method of claim 47, wherein the disorder is an angiogenic disorder.

58. The method of claim 47, wherein the disorder is a hematologic disorder.

59. The method of claim 47, wherein the disorder is a myeloproliferative disorder.

60. The method of claim 47, wherein the disorder is selected from a group consisting of diabetes, a cancer, an eye disease, an inflammation, psoriasis, or a viral infection.

61. The method of claim 60, wherein the cancer is selected from a group consisting of an alimentary/gastrointestinal tract cancer, colon cancer, liver cancer, skin cancer, breast cancer, ovarian cancer, prostate cancer, lymphoma, leukemia, kidney cancer, lung cancer, muscle cancer, bone cancer, bladder cancer and brain cancer.

62. The method of claim 47, wherein the disorder is selected from a group consisting of ocular neovasculariaztion, infantile haemangiomas; organ hypoxia, vascular hyperplasia, organ transplant rejection, lupus, multiple sclerosis, rheumatoid arthritis, psoriasis, Type I diabetes and complications from diabetes, inflammatory disease, acute pancreatitis, chronic pancreatitis, asthma, allergies, adult respiratory distress syndrome, cardiovascular disease, liver disease, other blood disorders, asthma, rhinitis, atopic, dermatitits, autoimmune thryroid disorders, ulerative colitis, Crohn's disease, metastatic melanoma, Kaposi's sarcoma, multiple myeloma, conditions associated with cytokines, and other autoimmune diseases including glomerulonephritis, scleroderma, chronic thyroiditis, Graves' disease, autoimmune gastritis, autoimmune hemolytic anemia, autoimmune neutropenia, thrombocytopenia, allergic asthma, atopic dermatitis, allergic rhinitis, chronic active hepatitis, myasthenia gravis, multiple sclerosis, inflammatory bowel disease, graft vs host disease, motor neuron disease, Alzheimer's disease, Parkinson's disease, amyotrophic lateral scelerosis, Huntington's disease, cerebral ischemia, or neurodegenerative disease caused by traumatic injury, strike, gluatamate neurtoxicity, hypoxia; ischemic/reperfusion injury in stroke, myocardial ischemica, renal ischemia, heart attacks, cardiac hypertrophy, atherosclerosis and arteriosclerosis, organ hyoxia, platelet aggregation, allergic contact dermatitis, hypersensitivity pneumonitis, systemic lupus erythematosus, juvenile arthritis, Sjogren's Syndrome, scleroderma, polymyositis, ankylosing spondylitis, psoriatic arthritis, Epstein Barr Virus, Hepatitis B, Hepatitis C, HIV, HTLV1, Vaicella-Zoster Virus, Human Papilloma Virus, food allergy, cutaneous inflammation, and immune suppression induced by solid tumors.

63. The method of claim 47, wherein the disorder is associated with a kinase.

64. The method of claim 47, wherein the disorder is associated with gatekeeper mutations in the kinase.

65. A pharmaceutical composition comprising at least one compound of claim 1 or 2, or pharmaceutically acceptable N-oxide(s), salts, hydrates, solvates, crystal forms and individual diastereomers thereof, and a pharmaceutically acceptable carrier therefore.

66. An article of manufacture comprising packaging material and a pharmaceutical composition contained within the packaging material, wherein the packaging material comprises a label which indicates that the pharmaceutical composition can be used for treatment of angiogenic-associated disorders, and wherein the pharmaceutical composition comprises at least one compound of claim 1 or 2, or pharmaceutically acceptable N-oxide(s), salts, hydrates, solvates, crystal forms and individual diastereomers thereof.

67. An article of manufacture comprising packaging material and a pharmaceutical composition contained within the packaging material, wherein the packaging material comprises a label which indicates that the pharmaceutical composition can be used for treatment of myeloproliferative disorder, proliferative diabetic retinopathy, a cancer, eye disease, inflammation, psoriasis, or a viral infection, and wherein the pharmaceutical composition comprises at least one compound of claim 1 or 2, or pharmaceutically acceptable N-oxide(s), salts, hydrates, solvates, crystal forms and individual diastereomers thereof.

68. The article of manufacture of claim 67, wherein the disorder is selected from a group consisting of an alimentary/gastrointestinal tract cancer, colon cancer, liver cancer, skin cancer, breast cancer, ovarian cancer, prostate cancer, lymphoma, leukemia, kidney cancer, lung cancer, muscle cancer, bone cancer, bladder cancer and brain cancer.

69. A method for reducing or eliminating resistance of a protein associated with a disorder, to currently used therapies, comprising synthesizing a compound of claim 1 or 2, wherein said compound is effective as an inhibitor of said protein, thereby overcome said resistance.
